(19) United States
(12) Patent Application Publication
(10) Pub. No.: US 2013/0136689 A1
(43) Pub. Date: May 30, 2013

(54) PROTEINS

(71) Applicants: Christian Rohlff, Abingdon (GB); Alasdair Stamps, Abingdon (GB)

(72) Inventors: Christian Rohlff, Abingdon (GB); Alasdair Stamps, Abingdon (GB)

(21) Appl. No.: 13/713,142

(22) Filed: Dec. 13, 2012

Related U.S. Application Data
(63) Continuation-in-part of application No. 12/547,772, filed on Aug. 26, 2009, now abandoned.
Application No. 12/547,772 is a continuation of application No. PCT/GB2008/050125, filed on Feb. 26, 2008.

(30) Foreign Application Priority Data
Jun. 5, 2007 (EP) PCT/EP2007/055537
Feb. 25, 2008 (GB) PCT/GB2008/050124

(51) Int. Cl. G01N 33/68

(52) U.S. Cl. CPC G01N 33/6893
USPC 424/1.49; 424/174.1; 424/178.1; 424/156.1; 424/133.1; 424/136.1; 514/19.3; 435/7.1; 435/7.92

(57) ABSTRACT

The present invention relates to the identification of membrane proteins associated with B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma which have utility as markers and for treatment of said cancers and which also form biological targets against which antibodies such as therapeutic antibodies (or other affinity reagents) or other pharmaceutical agents can be made.

### CROSS REFERENCE TO RELATED APPLICATION

The present invention is a Continuation in Part of U.S. application Ser. No. 12/547,772, filed Aug. 26, 2009, which is a Continuation of PCT Application No. PCT/GB2008/050125 filed Feb. 26, 2008, which in turn, claims priority from PCT Application Nos. PCT/EP2007/055537 filed Jun. 5, 2007 and PCT/GB2008/050124 filed Feb. 25, 2008, and U.S. Provisional Application Ser. Nos. 60/903,509 and 60/903,510, both filed Feb. 26, 2007. Priority under 35 U.S.C. §§119 and 120 is claimed, and the entire contents of each of the above applications are incorporated herein by reference in their entireties.

### INTRODUCTION

The present invention relates to the identification of membrane proteins associated with B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma which have utility as markers for B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma and breast cancer, cervical cancer, colorectal cancer, gastric cancer, hepatocellular carcinoma, lung cancer, melanoma, neuroblastoma, osteosarcoma, ovarian cancer, pancreatic cancer, prostate cancer, renal cell cancer and retinoblastoma metastases and which also form biological targets against which antibodies such as therapeutic antibodies (or other affinity reagents) or other pharmaceutical agents can be made.

### BACKGROUND OF THE INVENTION

### Acute Lymphocytic Leukaemia

Each year, about 35,000 new cases of all types of leukaemia are diagnosed in the USA. Of these, about 4,000 will be acute lymphocytic leukaemia (ALL). Although this is a leukaemia that occurs mostly in children, about one-third, or 1300 cases, will occur in adults. About 1,500 people will die of ALL in the USA each year; two-thirds of them will be adults. The risk of ALL is lowest between the ages of 25 and 50 and then begins to pick up.

### Acute Lymphocytic Leukaemia Diagnosis

Diagnostic tests for ALL include blood cell count, bone marrow aspiration, bone marrow biopsy, excisional lymph node biopsy, blood chemistry tests and lumbar puncture. Other lab tests include routine microscopic exam, cytochemistry, flow cytometry, immunocytochemistry, cytogenetics, molecular genetic studies, and gene-expression profiling. Imaging tests such as chest x-ray, computed tomography (CT) scans, magnetic resonance imaging (MRI), gallium scans, bone scans, and ultrasound may also be carried out.

### Acute Lymphocytic Leukaemia Staging

Leukaemia involves all the bone marrow and, in many cases, it has also spread to other organs. Lab tests focus on finding out the exact type (and subtype) of leukaemia. This in turn helps predict which treatments will work best and the prognosis for the patient.

There are 3 subtypes for ALL according to the French-American-British (FAB) classification. The original FAB system was based only on the way the leukaemic cells looked under the microscope after they were routinely processed or cytochemically stained. More recently, doctors have found that cytogenetic studies, flow cytometry, and molecular genetic studies provide more information that is sometimes useful in classifying ALL and predicting the patient's prognosis. Now the subtypes of ALL are: early pre-B ALL, common ALL, pre-B-cell ALL, mature B-cell ALL (Burkitt leukaemia), pre-T-cell ALL and mature T-cell ALL. T-cell ALL has the best prognosis, mature B-cell ALL the worst, and pre-B-cell ALL is intermediate.

One of the most important factors that affects outcome is a translocation between chromosomes 9 and 22 (Philadelphia chromosome). People with this translocation (20% to 25%) have a worse outcome than those without it. Another translocation that carries a poor outlook is one between chromosomes 4 and 11, which occurs in about 5% of patients.

### Acute Lymphocytic Leukaemia Treatment

Chemotherapy is the major treatment for ALL. Treatments are given in the following phases: remission induction, consolidation, maintenance therapy and central nervous system (CNS) prophylaxis. Chemotherapeutic agents used for remission induction and consolidation include cyclophosphamide, vincristine, dexamethasone or prednisone, L-asparaginase and doxorubicin (Adriamycin) or daunorubicin. Maintenance therapy consists of methotrexate with 6-mercaptopurine (6-MP), often combined with vincristine and prednisone. CNS prophylaxis involves methotrexate and/or cytarabine. In general, about 80% of patients will respond completely to these treatments. Unfortunately, about half of these patients relapse, so the overall cure rate is around 30%. If leukaemias recur after treatment, they will most often do so in the bone marrow and blood. Occasionally, the brain or spinal fluid will be the first place they recur. If the leukaemia is refractory (which happens in about 15%-20% of cases) then newer or more intensive doses of drugs may be tried, although they are less likely to work. A stem cell transplant may be attempted if the leukaemia can be put into remission. It is possible for a patient with recurrent leukaemia to go into remission again, although it may be only temporary. In this situation, a stem cell transplant is considered after more induction chemotherapy. If the leukaemia is persistent, eventually chemotherapy treatment becomes unhelpful.

Radiation therapy is sometimes used to treat leukaemia that has spread to the brain and spinal fluid or to the testicles. Radiation to the whole body is often an important part of treatment before bone marrow or peripheral blood stem cell transplantation.

Clinical trials are being conducted to see whether better outcomes are achieved using a combination of chemotherapy and imatinib mesylate (Gleevec), a drug which targets cells that have the Philadelphia chromosome (9-22 translocation or bcr-abl gene fusion). Another drug, dasatinib, approved for treatment of ALL, has the same mode of action as imatinib but appears to be more potent and can act against leukaemia cells that have become resistant to imatinib.

### Non-Hodgkin's Lymphoma

Non-Hodgkin's lymphoma (NHL) is a cancer of lymphoid tissue. In the USA, 85% of all cases of non-Hodgkin's lymphoma derive from B lymphocytes (B-cell) and 15% from T lymphocytes (T-cell). There are about 59,000 new cases of NHL in the USA each year, with around 19,000 deaths. This cancer is more common in men than in women and whites are affected more often than African or Asian people. A person's risk of getting NHL during his or her lifetime is 1 in 50. The risk of dying of this disease is about 1 in 100. Since the early 1970s, incidence rates for non-Hodgkin lymphoma have nearly doubled. More recently, incidence rates have stabilized due, perhaps, to the decline in AIDS-related NHL.

### B-Cell Lymphomas:

About 33% of all non-Hodgkin's lymphomas in the USA are diffuse large B-cell lymphomas. About 14% are follicular lymphomas. Chronic lymphocytic leukaemia (CLL) and small lymphocytic lymphoma (SLL) account for 24% of all lymphomas. Only about 2% of lymphomas are mantle cell lymphomas. All marginal zone lymphomas account for about 4% of lymphomas. Primary mediastinal B-cell lymphoma accounts for about 2% of all lymphomas. Burkitt's lymphoma makes up about 1% to 2% of all lymphomas. Lymphoplasmocytic lymphoma (Waldenstrom macroglobulinemia) accounts for 1-2% of lymphomas. Hairy cell leukaemia is rare—about 1,000 people in the USA are diagnosed with this type each year. Although primary central nervous system (CNS) lymphoma was a rare tumour in the past, it has become more common in patients with AIDS.

### Non-Hodgkin's Lymphoma Diagnosis

NHL may cause many different signs and symptoms, depending on where it is found in the body. A biopsy is the only way to tell for sure if cancer is present. Types of biopsy include excisional or incisional biopsy, fine needle aspiration (FNA) biopsy, bone marrow aspiration and biopsy, and lumbar puncture. Lab tests including immunohistochemistry, flow cytometry, cytogenetics, molecular genetic studies and blood tests can also be performed. Imaging tests that may be used include chest x-ray, computed tomography (CT) scan, magnetic resonance imaging (MRI) scan, positron emission tomography (PET) scan, gallium scan, bone scan, and ultrasound.

### Non-Hodgkin's Lymphoma Staging

Survival statistics vary widely by cell type and stage of disease at the time of diagnosis. However, the overall 5-year relative survival rate for people with non-Hodgkin's lymphoma is 60%, and 10-year relative survival is 49%.

Non-Hodgkin's lymphoma is staged using the Ann Arbor staging system stages I-IV. The International Prognostic Index (IPI) helps predict how quickly the lymphoma might grow and how well a patient might respond to treatment. It is mainly used in patients with fast growing lymphomas. Over 75% of people in the lowest group will live longer than 5 years, whereas only 30% of people in the highest group live 5 years.

### Survival Rates for B-Cell Lymphomas:

Diffuse large B-cell lymphoma can be cured in around 40% to 50% of patients. Follicular lymphomas are not considered curable but are slow growing, and the 5-year survival rate is around 60% to 70%. Over time, about one third of follicular lymphomas change into a fast growing diffuse B-cell lymphoma. Chronic lymphocytic leukaemia (CLL) and small lymphocytic lymphoma (SLL) are not considered curable but depending on the stage and growth rate of the disease, most patients can live well over 10 years with this lymphoma. Only 20% of patients with mantle cell lymphoma survive at least 5 years. Marginal zone lymphomas are often curable. About half of patients with primary mediastinal B-cell lymphoma can be cured. Although Burkitt's lymphoma is a fast growing lymphoma, over half of patients can be cured by intensive chemotherapy. Although lymphoplasmocytic lymphoma (Waldenstrom macroglobulinemia) isn't curable, most patients live longer than 5 years. Hairy cell leukaemia can usually be treated successfully. The outlook for people with primary CNS lymphoma is poor but about 30% to 50% of people can live at least 5 years.

### Non-Hodgkin's Lymphoma Treatment

Surgery is not often used to treat NHL. It has been used to treat lymphomas that start in organs such as the stomach or thyroid, but only if it has not spread beyond these organs. External beam radiation therapy is often the main treatment for early stage lymphomas, and is often used along with chemotherapy. Chemotherapeutic drugs used include a combination of cyclophosphamide, doxorubicin, vincristine and prednisone known as CHOP, chlorambucil, fludarabine, and etoposide. Immunotherapy using either interferon or monoclonal antibodies such as rituximab can also sometimes be used as a treatment. Bone marrow or peripheral blood stem cell transplantation (SCT) is used for patients when standard treatment has not worked.

### Treatment of B-Cell Lymphomas:

The main treatment for diffuse large B-cell lymphoma is chemotherapy with CHOP with the addition of rituximab. Radiation therapy may also be added. Follicular lymphoma has not been shown to be curable by any of the standard treatments. Radiation therapy, chemotherapy and/or monoclonal antibodies can be used, with the point of therapy being to control the disease for as long as possible while causing the fewest side effects. Chronic lymphocytic leukaemia (CLL) and small lymphocytic lymphoma (SLL) are also not considered curable and the treatment is the same as for follicular lymphoma. There is also no curative treatment for mantle cell lymphoma which is often fatal. Radiation therapy and chemotherapy are used to treat extranodal marginal zone B-cell lymphomas. Nodal marginal zone B-cell lymphoma and splenic marginal zone B-cell lymphoma are generally low-grade lymphomas and are treated with either observation or low-intensity chemotherapy. Primary mediastinal B-cell lymphoma is treated like localized diffuse large B-cell lymphoma. Burkitt's lymphoma is a very fast growing lymphoma that is treated intensely with chemotherapy. The main treatment for lymphoplasmocytic lymphoma (Waldenstrom macroglobulinemia) is chemotherapy or rituximab. Hairy cell leukaemia is a slow growing lymphoma that invades the spleen and lymph nodes as well as the blood and can be treated with chemotherapy.

### Breast Cancer

Globally, breast cancer is both the most common cancer (10% of all cancer cases) and the leading cause of cancer death (6% of cancer deaths) in women. Global incidence of breast cancer is over 1 million cases per year, with about 400,000 deaths. Women in North America have the highest rate of breast cancer in the world (over 200,000 new cases per year, with about 40,000 deaths). The chance of developing invasive breast cancer at some time in a woman's life is about 1 in 8. Breast cancer incidence increases with age, rising sharply after age 40. In the USA, about 77% of invasive breast cancers occur in women over age 50. It has been estimated that approximately US$8.1 billion is spent in the USA each year on treating breast cancer.

### Breast Cancer Diagnosis

Early diagnosis improves the likelihood that treatment will be successful. Screening methods such as mammograms, clinical breast examinations and breast self-examinations are useful in detecting breast cancer. Current diagnostic methods include breast ultrasound, ductogram, full-field digital mammography (FFDM), scintimammography and MRI. A biopsy (fine needle aspiration biopsy, core biopsy or surgical biopsy) is then performed to confirm the presence of breast cancer. Imaging tests such as a chest x-ray, bone scan, CT, MRI and PET are used to detect if the breast cancer has spread.

### Breast Cancer Staging

Breast cancer is staged using the American Joint Committee on Cancer (AJCC) TNM system—Stage 0-Stage IV. Ductal carcinoma in situ (DCIS), a non-invasive cancer which accounts for 20% of new breast cancer cases is Stage 0. Nearly all women diagnosed at this early stage of breast cancer can be cured. Infiltrating (invasive) ductal carcinoma (IDC), which accounts for 80% of invasive breast cancer and infiltrating (invasive) lobular carcinoma (ILC), which accounts for 5% of invasive breast cancers are more severe Stage I-IV cancers and can metastasize.

### Breast Cancer Treatment

Breast-conserving surgery (lumpectomy) or mastectomy are the usual treatments for breast cancer. For stage I or II breast cancer, breast-conserving surgery is as effective as mastectomy. Patients can then undergo reconstructive surgery. Axillary lymph node sampling and removal or sentinel lymph node biopsy (SLNB) is performed to see if the cancer has spread to the lymph nodes.

Neoadjuvant chemotherapy can be given before surgery to shrink large cancers. Adjuvant chemotherapy after surgery reduces the risk of breast cancer recurrence. Chemotherapy can also be used as the main treatment for women whose cancer has spread outside the breast and underarm area. Chemotherapeutic agents used include anthracyclines (e.g. methotrexate, fluorouracil, doxorubicin, epirubicin), taxanes (e.g. paclitaxel, docetaxel, vinorelbine) and alkylating agents (e.g. cyclophosphamide).

Radiation therapy (usually external beam radiation but sometimes brachytherapy) is given once chemotherapy is complete.

Hormone therapy with selective estrogen receptor modulators (e.g. tamoxifen) can be given to women with estrogen receptor positive breast cancers. Taking tamoxifen after surgery for 5 years can reduce recurrence by about 50% in women with early breast cancer. Aromatase inhibitors such as exemestane, letrozole or anastrozole can also be used.

Women with HER2 positive cancers (about ⅓ of breast cancers) can be given biological response modifiers such as trastuzumab (Herceptin). Clinical trials have shown that adding trastuzumab to chemotherapy lowers the recurrence rate and death rate over chemotherapy alone after surgery in women with HER2 positive early breast cancers.

### Breast Cancer Survival by Stage

Patients diagnosed with breast cancer between 1995 and1998 had a 5 year relative survival rate of 100% for stage 0 and I, 92% for stage IIA, 81% for stage IIB, 67% for stage IIIA, 54% for stage IIIB and 20% for stage IV.

### Cervical Cancer

Cervical cancer is second only to breast cancer as the most common malignancy in both incidence and mortality and remains a significant public health problem throughout the world. In the USA alone, invasive cervical cancer accounts for approximately 19% of all gynecological cancers. In the USA, about 9,710 cases of invasive cervical cancer are diagnosed each year, with 3,700 deaths. Non-invasive cervical cancer (carcinoma in situ) is about 4 times more common than invasive cervical cancer. Between 1955 and 1992, the number of cervical cancer deaths in the United States dropped by 74%. The main reason for this change is the increased use of the Pap test screening procedure. The death rate from cervical cancer in the USA continues to decline by nearly 4% a year. Half of women diagnosed with this cancer are between the ages of 35 and 55. Cervical cancer occurs most often in Hispanic women; the rate is over twice that in non-Hispanic white women. African-American women develop this cancer about 50% more often than non-Hispanic white women. In many developing countries, where mass screening programs are not widely available, the clinical problem is more serious. Worldwide, the number of new cases is estimated to be 471,000 with a four-year survival rate of only 40% (Munoz et al., 1989, Epidemiology of Cervical Cancer in: “Human Papillomavirus”, New York, Oxford Press, pp 9-39; National Institutes of Health, Consensus Development Conference Statement on Cervical Cancer, Apr. 1-3, 1996). These cases are usually diagnosed at an invasive late stage, rather than as precancers or early cancers.

### Cervical Cancer Diagnosis

Early detection greatly improves the chances of successful treatment and prevents any early cervical cell changes from becoming cancerous. Although the Pap test is the most cost-effective cancer screening test developed to date (Greenberg, M. D., et al., 1995, Clin Obstet Gynecol 38(3): 600-9), it is not perfect. One of its limitations is that Pap tests are examined by humans, so an accurate analysis of the hundreds of thousands of cells in each sample is not always possible. It was reported that the mean sensitivity of primary Pap tests is approximately 58% and the accuracy of a repeat test is only about 66% (Fahey M. T., et al., 1995, Am. J. Epidemiol. 141: 680-689). The low sensitivity and poor reproducibility have complicated the management of ASCUS (atypical squamous cells of undetermined significance) and LSIL (low-grade squamous intraepithelial lesion) patients. If an “accelerated repeat Pap test” is recommended for the follow-up of women with primary diagnosis of ASCUS or LSIL, patients will risk delay in diagnosis of potential high-grade lesions. However, if these patients are universally referred to colposcopy, the vast majority of women will be over treated. Only 5-10% of women with ASCUS have high-grade disease upon colposcopy, and more than 80% of LSIL will regress to normal or stay in their current state (Cox, J. T., 2000, Clinics in Laboratory Medicine. 20 (2): 303-343, Ostor A. G., 1993, Int. J. Gynecol. Pathol. 12 (2): 186-192). New tests can identify HPVs by finding their DNA in the cells. Many doctors are now testing for HPV if the Pap test result is mildly abnormal. However, since the vast majority of HPV infections and the resulting squamous intraepithelial lesions regress spontaneously, especially in young women, HPV testing cannot specifically identify patients whose lesions will persist or progress to invasive carcinoma (Sasieni, P. D., 2000, J. Am. Med. Women Assoc. 55 (4): 216-219, Sasieni, P. D., 2000, Br. J. Cancer, 83 (5): 561-565). A vaccine (Gardisil) has been approved for use by FDA and it protects against HPV types 16, 18, 6, and 11. The vaccine does not protect against all cancer-causing types of HPV, so Pap tests are still necessary. Other tests are required to diagnose cervical cancer following the Pap test including a colposcopy and biopsy, and sometimes an endocervical scraping. The biopsy can be either a colposcopic biopsy, an endocervical curettage or a cone biopsy—LEEP (LLETZ) or cold knife cone biopsy. Imaging tests such as a chest x-ray, computed tomography (CT), magnetic resonance imaging (MRI) and positron emission topography (PET) can also be used.

### Cervical Cancer Staging

Cervical cancer is staged with the FIGO (International Federation of Gynecology and Obstetrics) System of Staging—0-IV. The overall (all stages combined) 5-year survival rate for cervical cancer is about 73%.

### Cervical Cancer Treatment

For pre-invasive cancer, cryosurgery, laser surgery or conisation can be used as treatment. For Stage I-IIA cervical cancer, a hysterectomy is the usual treatment. A trachelectomy may be possible in some cases. For recurrent cervical cancer, a pelvic exenteration is usually performed. Radiation therapy (either external beam radiation therapy or brachytherapy) is an option for Stage IB-Stage IV patients. Combining radiation therapy with chemotherapy has been found to be more effective than radiation therapy alone. Chemotherapeutic agents used include cisplatin, paclitaxel, topotecan, ifosfamide, and fluorouracil. Stage IVB cervical cancer is usually not considered curable but a combination of radiation therapy and chemotherapy can help relieve symptoms.

### Cervical Cancer Survival by Stage

Womean who were treated more than 10 years ago had 5 year survival rates of above 95% for stage IA, around 90% for stage IB1, around 80-85% for stage IB2, around 75-78% for stage IIA/B, around 47-50% for stage IIIA/B and around 20-30% for stage IV.

### Chronic Lymphocytic Leukaemia

Chronic lymphocytic leukaemia (CLL) is the most common adult leukaemia in the Western Hemisphere, and is generally fatal once the disease progresses. There are probably two different kinds of CLL. One is very slow growing and rarely needs to be treated. People with this kind of CLL survive on average 13 to 15 years. The other kind of CLL is faster growing and is a more serious disease. People with this form of CLL survive only about 6 to 8 years. About 9,800 new cases of CLL are diagnosed each year in the USA, with about 4,600 deaths. CLL affects only adults. The average age of patients is about 70 and it is rarely seen in people under the age of 40.

### Chronic Lymphocytic Leukaemia Diagnosis

Diagnostic tests for chronic lymphocytic leukaemia carried out include blood cell count, bone marrow aspiration, bone marrow biopsy, excisional lymph node biopsy, blood chemistry tests and lumbar puncture. Other lab tests include routine microscopic exam, cytochemistry, flow cytometry, immunocytochemistry, cytogenetics, molecular genetic studies, and fluorescent in situ hybridization (FISH). Imaging tests may also be carried out including chest x-rays, computed tomography (CT) scans, magnetic resonance imaging (MRI) and ultrasound.

### Chronic Lymphocytic Leukaemia Staging

The prognosis of a patient with leukaemia depends on the leukaemia's type or subtype, cellular features determined by lab tests, and results of imaging studies.

There are 2 different systems for staging CLL. The Rai classification is used more often in the USA, whereas the Binet system is used more widely in Europe.

The 5 Rai system stages can be separated into low-, intermediate-, and high-risk categories. Stage 0 is considered low risk, stages I and II are considered intermediate risk, and stages III and IV are considered high risk.

The Binet staging system uses the letters A (least severe), B, and C (most severe) to define the stages. CLL is classified according to the number of affected lymphoid tissue groups and the presence of anaemia or thrombocytopenia.

Other markers such as chromosome changes, mutational status, ZAP-70 and CD38 status are also used to distinguish between the two different types of CLL.

### Chronic Lymphocytic Leukaemia Treatment

The prognosis for patients with low-risk CLL is very good. Treatment is only considered if there are signs that the leukaemia is progressing. When indicated, initial treatment is usually chemotherapy. Chlorambucil (Leukeran) and fludarabine (Fludara) are the chemotherapy drugs most commonly used to treat CLL.

Patients with intermediate- and high-risk CLL who do not have any symptoms may not need treatment right away. Some with very high-risk disease may best be treated with early stem cell transplantation. No specific treatment has been shown to improve survival. In general, chemotherapy is used as treatment. Monoclonal antibodies such as rituximab can be used alongside chemotherapy. Alemtuzumab (Campath) is used in patients with CLL who are no longer responding to standard chemotherapy treatments.

### Colorectal Cancer

Colorectal cancer (CRC) is one of the leading causes of cancer-related morbidity and mortality, responsible for an estimated half a million deaths per year, mostly in Western, well developed countries. In these territories, CRC is the third most common malignancy (estimated number of new cases per annum in USA and EU is approximately 350,000 per year). Estimated healthcare costs related to treatment for colorectal cancer in the United States are more than $8 billion.

### Colorectal Cancer Diagnosis

Today, the fecal occult blood test and colonoscopy, a highly invasive procedure, are the most frequently used screening and diagnostic methods for colorectal cancer. Other diagnostic tools include Flexible Sigmoidoscopy (allowing the observation of only about half of the colon) and Double Contrast Barium Enema (DCBE, to obtain X-ray images).

### Colorectal Cancer Staging

CRC has four distinct stages: patients with stage I disease have a five-year survival rate of >90%, while those with metastatic stage IV disease have a <5% survival rate according to the US National Institutes of Health (NIH).

### Colorectal Cancer Treatment

Once CRC has been diagnosed, the correct treatment needs to be selected. Surgery is usually the main treatment for rectal cancer, although radiation and chemotherapy will often be given before surgery. Possible side effects of surgery include bleeding from the surgery, deep vein thrombosis and damage to nearby organs during the operation.

Currently, 60 percent of colorectal cancer patients receive chemotherapy to treat their disease; however, this form of treatment only benefits a few percent of the population, while carrying with it high risks of toxicity, thus demonstrating a need to better define the patient selection criteria.

Colorectal cancer has a 30 to 40 percent recurrence rate within an average of 18 months after primary diagnosis. As with all cancers, the earlier it is detected the more likely it can be cured, especially as pathologists have recognised that the majority of CRC tumours develop in a series of well-defined stages from benign adenomas.

### Colon Cancer Survival by Stage:

For stage 193%, for stage IIA 85%, for stage IIB 72%, for stage IIIA 83%, for stage IIIB 64%, for stage IIIC 44% and for stage IV 8%.

### Gastric Cancer

Gastric cancer is the second-leading cause of cancer-related deaths in the world, with about 700,000 deaths per year, mostly in less developed countries. In the USA, about 22,000 people are diagnosed with gastric cancer each year, with about 11,000 deaths. This figure is approximately ten times higher in Japan. Two thirds of people diagnosed with gastric cancer are older than 65.

### Gastric Cancer Diagnosis

Early stage gastric cancer rarely causes symptoms so only about 10-20% of gastric cancers in the USA are found in the early stages, before they have spread to other areas of the body. Studies in the USA have not found mass screening for gastric cancer to be useful because the disease is not that common. Endoscopy followed by a biopsy is the main procedure used to diagnose gastric cancer. Other diagnostic methods include barium upper gastrointestinal radiographs, endoscopic ultrasound, CT scan, PET scan, MRI scan, chest x-ray, laparoscopy, complete blood count (CBC) test and fecal occult blood test.

### Gastric Cancer Staging

Gastric cancer is staged using the American Joint Commission on Cancer (AJCC) TNM system—Stage 0-Stage IV. Patients with stage 0 disease have a 5-year survival rate of >90%, while there is usually no cure for patients with stage IV disease where the 5-year survival rate is only 7%. The overall 5-year relative survival rate of people with gastric cancer in the USA is about 23%. The 5-year survival rate for cancers of the proximal stomach is lower than for cancers in the distal stomach.

### Gastric Cancer Treatment

Surgery is the only way to cure gastric cancer. There are three types of surgery used—endoscopic mucosal resection (only for early stage gastric cancer), subtotal gastrectomy or total gastrectomy. Gastric cancer often spreads to lymph nodes so these must also be removed. If the cancer has extended to the spleen, the spleen is also removed. Surgery for gastric cancer is difficult and complications can occur.

Chemotherapy may be given as the primary treatment for gastric cancer that has spread to distant organs. Chemotherapy together with external beam radiation therapy may delay cancer recurrence and extend the life span of people with less advanced gastric cancer, especially when the cancer could not be removed completely by surgery. Chemotherapeutic agents used include fluorouracil, doxorubicin, methotrexate, etoposide and cisplatin. More recently, imatinib mesylate (Gleevec) has been trialled in gastrointestinal stromal tumours (GIST), improving progression free survival.

### Gastric Cancer Survival by Stage:

For stage 0 greater than 90%, for stage IA 80% for stage IB 60%, for stage II 34%, for stage IIIA 17%, for stage IIIB 12% and stage IV 7%.

### Glioblastoma

Glioblastoma, also known as glioblastoma multiforme, may develop from a diffuse astrocytoma or an anaplastic astrocytoma but more commonly presents de novo without evidence of a less malignant precursor. Histologically, this tumour is an anaplastic, cellular glioma composed of poorly differentiated, often pleomorphic astrocytic tumour cells with marked nuclear atypia and brisk mitotic activity. Glioblastoma primarily affects the cerebral hemispheres. CNS tumours are associated with characteristic patterns of altered oncogenes, altered tumour-suppressor genes, and chromosomal abnormalities.

Glioblastoma accounts for approximately 12% to 15% of all brain tumours (which account for 85% to 90% of all primary central nervous system (CNS) tumours). New cases for CNS tumours in USA are approximately 18,800 (6.6 per 100,000 persons) per year, with around 12,800 (4.7 per 100,000 persons) deaths. This type of cancer accounts for approximately 1.3% of all cancers and 2.2% of all cancer-related deaths in the USA. Worldwide, there are approximately 176,000 new cases of brain and other CNS tumours per year, with an estimated mortality of 128,000. In general, the incidence of primary brain tumours is higher in whites than in blacks, and mortality is higher in males than in females. The peak incidence occurs between the ages of 45 and 70 years.

Primary brain tumours rarely spread to other areas of the body, but they can spread to other parts of the brain and to the spinal axis. Most patients with central nervous system (CNS) neoplasms do not live long enough to develop metastatic disease.

### Glioblastoma Diagnosis

Computed tomography (CT) and magnetic resonance imaging (MRI) have complementary roles in the diagnosis of CNS neoplasms. Angiography can also be used in diagnosis. In post-therapy imaging, single-photon emission computed tomography (SPECT) and positron emission tomography (PET) may be useful in differentiating tumour recurrence from radiation necrosis. A definite diagnosis is then made by performing a biopsy.

### Glioblastoma Staging

Glioblastomas are among the most aggressively malignant human neoplasms, with a mean total length of disease in patients with primary glioblastoma of less than 1 year. On the WHO classification of nervous system tumours from grade I to grade IV, glioblastoma is classified as grade IV. The 5-year survival rate for patients with glioblastoma aged over 45 is 2% or less.

### Glioblastoma Treatment

The cure rate for glioblastoma is very low with standard local treatment. The first step in most cases is surgical removal by craniotomy of as much of the tumour as is safe without destroying normal function. Glioblastomas are not cured by surgery because cells from the tumour invade deeply the surrounding normal brain tissue. However, surgery reduces the pressure of the tumour against the rest of the brain and can prolong life.

Radiation therapy (external beam radiation, interstitial radiotherapy, 3D conformal therapy, stereotactic radiosurgery or brachytherapy) can increase the cure rate or prolong disease-free survival. Radiation therapy may also be useful in the treatment of recurrences in patients initially treated with surgery alone. Therapy can involve surgically implanted carmustine-impregnated polymer combined with postoperative external-beam radiation therapy (EBRT).

Chemotherapy is usually given along with or following radiation therapy and may prolong survival. Chemotherapeutic agents used include temozolomide, BCNU (carmustine) and cisplatin. Growth factor inhibitors erlotinib (Tarceva) and gefitinib (Iressa) have been shown to shrink tumours in some patients.

Novel biologic therapies under clinical evaluation for patients with brain tumours include dendritic cell vaccination, tyrosine kinase receptor inhibitors, farnesyl transferase inhibitors, viral-based gene therapy, and oncolytic viruses.

### Hepatocellular Carcinoma (HCC)

Hepatocellular carcinoma (HCC) arises from the main cells of the liver (the hepatocytes) and accounts for around 80% of all cases of liver cancer. It is usually confined to the liver and is associated with cirrhosis in 50% to 80% of patients. Hepatocellular carcinoma is about 3 times more common in males than in females. Chronic infection with hepatitis B virus (HBV) or hepatitis C virus (HCV) is a major cause of HCC and is responsible for making liver cancer the most common cancer in many parts of the world. In the United States, hepatitis C infection is responsible for about 50% to 60% of all liver cancers and hepatitis B is responsible for another 20%. Exposure to Aflatoxins is also a cause of HCC, mostly in warmer and tropical countries. Liver cancer accounts for about 5.8% of all cancer cases globally (about 626,000 cases) and 8.9% of deaths per year (about 598,000). It is the 3rd most common cause of cancer-related death in both men and women worldwide. HCC is predominantly found in Asia and Africa, which account for 80% of cases. In the USA, there are approximately 18,500 new cases of HCC and 16,000 deaths per year. About 85% of people diagnosed with liver cancer are between 45 and 85 years of age. About 4% are between 35 and 44 years of age and only 2.4% are younger than 35.

### Hepatocellular Carcinoma Diagnosis

Since symptoms of liver cancer often do not appear until the disease is advanced, only a small number of liver cancers are found in the early stages and can be removed with surgery.

Many signs and symptoms of liver cancer are relatively nonspecific—that is, they can be caused by other cancers or by non-cancerous diseases. Imaging tests such as ultrasound, computed tomography (CT), magnetic resonance imaging (MRI) and angiography are commonly used to diagnose HCC. Other diagnostic tools include laparoscopy, biopsy, alpha-fetoprotein (AFP) blood test, liver function tests (LFTs), prothrombin time (PT) and tests for hepatitis B and C.

### Hepatocellular Carcinoma Staging

HCC has four stages, stage I to stage IV according to the American Joint Committee on Cancer (AJCC) TNM system. HCC can be classified as localized resectable, localized unresectable or advanced. The overall 5-year relative survival rate for liver cancer is about 9%.

One reason for this low survival rate is that most patients with liver cancer also have cirrhosis of the liver, which itself can be fatal (people with liver cancer and class C cirrhosis are generally too sick for any treatment and usually die in a few months). The 5 year survival for localized resectable HCC following surgery is between 40% and 70%. For advanced HCC there is no standard treatment and the 5 year survival rate is less than 5%. Survival continues to drop after diagnosis and treatment so that by 10 years it is less than 2.5%.

### Hepatocellular Carcinoma Treatment

Treatment of liver cancer depends on the size of the tumour and whether the patient has cirrhosis. At this time, surgery, either by resection or liver transplantation, offers the only chance to cure a liver cancer. People without cirrhosis can do well with surgical removal of the tumour.

However, in many cases, it might not be possible to safely remove a localized liver cancer. Less than 30% of the patients having explorative surgery are able to have their cancer completely removed by surgery. Partial hepatectomy results in a 5-year survival of 30% to 40%. If there is cirrhosis, or a very large tumour, most experts recommend liver transplantation as the main treatment. The 5-year survival for liver transplantation patients is around 70% but the opportunities for liver transplantation are limited.

Other treatments include radiofrequency ablation (RFA), ethanol ablation, cryosurgery, hepatic artery embolization, chemoembolization or three-dimensional conformal radiation therapy (3DCRT). Chemotherapy can also be used but shrinks fewer than 1 in 5 tumours. This may be improved by hepatic artery infusion (HAI). Chemotherapeutic agents used include Adriamycin, VP-16, Cisplatinum, Mitomycin, 5-FU and Leucovorin.

The prognosis for any treated primary liver cancer patient with progressing, recurring, or relapsing disease is poor. Treatment of liver cancer that returns after initial therapy depends on many factors, including the site of the recurrence, the type of initial treatment, and the functioning of the liver. Patients with localized resectable disease that recurs in the same spot may be eligible for further surgery.

### Lung Cancer

Lung cancer is the most common form of cancer worldwide (accounting for about 12% of cancer cases) and the main cause of death from cancer (accounting for about 18% of deaths). Global incidence of lung cancer is over 1,300,000 per year, with the number of deaths over 1,100,000. In the USA, there are about 170,000 new cases per year (about 13% of all cancers), with about 160,000 deaths (about 28% of cancer deaths). Lung cancer is much more prevalent among men than women. Nearly 70% of people diagnosed with lung cancer are older than 65; fewer than 3% of all cases are found in people under the age of 45. Around 15% of all lung cancers are small cell type (SCLC), which tend to spread widely through the body, while the remaining 85% are non-small cell (NSCLC). It has been estimated that approximately US$9.6 billion is spent in the USA each year on treating lung cancer.

### Lung Cancer Diagnosis

Lung cancer is a life-threatening disease because it often metastasises even before it can be detected on a chest x-ray. Usually symptoms of lung cancer do not appear until the disease is in an advanced stage. So far, there is no screening test that has been shown to improve a person's chance for a cure. Imaging tests such as a chest x-ray, CT scan, MRI scan or PET scan may be used to detect lung cancer. Tests to confirm the diagnosis are then performed and include sputum cytology, needle biopsy, bronchoscopy, endobronchial ultrasound and complete blood count (CBC).

### Lung Cancer Staging

Nearly 60% of people diagnosed with lung cancer die within one year of diagnosis; 75% die within 2 years. The 5-year survival rate for people diagnosed with NSCLC is about 15%; for SCLC the 5-year survival rate is about 6%. NSCLC is staged using the American Joint Committee on Cancer (AJCC) TNM system—Stage 0-Stage IV. The 5-year survival rates by stage are as follows: stage I: 47%; stage II; 26%; stage III: 8% and stage IV: 2%. SCLC has a 2-stage system—limited stage and extensive stage. About two thirds of SCLC patients have extensive disease at diagnosis. If SCLC is found very early and is localised to the lung alone, the 5-year survival rate is around 21%, but only 6% of patients fall into this category. Where the cancer has spread, the 5-year survival is around 11%. For patients with extensive disease, the 5-year survival is just 2%.

### Lung Cancer Treatment

Surgery is the only reliable method to cure NSCLC. Types of surgery include lobectomy, pneumonectomy, segmentectomy and video-assisted thoracic surgery (for small tumours). External beam radiation therapy is sometimes used as the primary treatment, especially if the patient's health is too poor to undergo surgery. Radiation therapy can also be used after surgery. Chemotherapy may be given as the primary treatment or as an adjuvant to surgery.

Targeted therapy using epidermal growth factor receptor (EGFR) antagonists such as gefitinib or erlotinib can also be given after other treatments have failed. Antiangiogenic drugs, such as bevacizumab, have been found to prolong survival of patients with advanced lung cancer. Photodynamic therapy is also being researched as a treatment for lung cancer.

The main treatment for SCLC is chemotherapy, either alone or in combination with external beam radiation therapy and very rarely, surgery.

Chemotherapeutic agents used for NSCLC and SCLC include cisplatin, carboplatin, mitomycin C, ifosfamide, vinblastine, gemcitabine, etoposide, vinorelbine, paclitaxel, docetaxel and irinotecan.

### Melanoma

Cancer of the skin is the most common of all cancers, probably accounting for more than 50% of all cancers. Melanoma accounts for about 4% of skin cancer cases but causes a large majority of skin cancer deaths. Half of all melanomas are found in people under age 57. About 1 of every 30,000 girls aged 15 to 19 will develop melanoma. For boys of this age, the rate is about 1 of every 15,000. In the USA, about 62,000 new melanomas are diagnosed each year, with around 8,000 deaths. The number of new melanomas diagnosed in the United States is increasing. Among white men and women in the United States, incidence rates for melanoma increased sharply at about 6% per year from 1973 until the early 1980s. Since 1981, however, the rate of increase slowed to little less than 3% per year. Since 1973, the mortality rate for melanoma has increased by 50%. More recently, the death rate from melanoma has leveled off for men and dropped slightly in women. The risk of melanoma is about 20 times higher for whites than for African Americans.

### Melanoma Diagnosis

Excisional biopsy is the preferred diagnostic method but other types of skin biopsy can also be used including incisional biopsy, shave biopsy and punch biopsy. Metastatic melanoma may not be found until long after the original melanoma was removed from the skin. Metastatic melanoma can be diagnosed using a number of methods including fine needle aspiration biopsy, surgical lymph node biopsy and sentinel lymph node mapping and biopsy. Imaging tests such as a chest x-ray, computed tomography (CT), magnetic resonance imaging (MRI), positron emission tomography (PET) and nuclear bone scans can also be used.

### Melanoma Staging

Melanoma is staged using the American Joint Committee on Cancer (AJCC) TNM system—Stage 0-Stage IV. The thickness of the melanoma is measured using the Breslow measurement.

### Melanoma Treatment

Thin melanomas can be completely cured by excision. If the melanoma is on a finger or toe, treatment may involve amputation of the digit. If the melanoma has spread to the lymph nodes, lymph node dissection may be required.

No current treatment is usually able to cure stage IV melanoma. Although chemotherapy is usually not as effective in melanoma as in some other types of cancer, it may relieve symptoms or extend survival of some patients with stage IV melanoma. Chemotherapy drugs often used to treat melanoma include dacarbazine, carmustine, cisplatin, vinblastine and temozolomide. Recent studies have found that biochemotherapy, combining several chemotherapy drugs with 1 or more immunotherapy drugs may be more effective than a single chemotherapy drug alone. Immunotherapy drugs include interferon-alpha and/or interleukin-2. Both drugs can help shrink metastatic (stage III and IV) melanomas in about 10% to 20% of patients. Interferon-alpha2b given to patients with stage III melanoma following surgery may delay the recurrence of melanoma. Isolated limb perfusion, using Melphalan, is an experimental type of chemotherapy sometimes used to treat metastatic melanomas confined to the arms or legs. Radiation therapy may be used to treat recurrent melanoma and is used as palliation of metastases to the bone and brain.

A person who has already had melanoma has an increased risk of developing melanoma again. In one study, about 11% of people with melanoma developed a second one within 5 years. And those that developed a second melanoma had a 30% chance of developing a third one in 5 years.

### Melanoma Survival by Stage

| Stage | 5-year relative survival rate | 10-year relative survival rate |
|---|---|---|
| 0 | 97% | — |
| I | 90-95% | 80% |
| IIA | 78% | 64% |
| IIB | 63-67% | 51-54% |
| IIC | 45% | 32% |
| IIIA | 63-70% | 57-63% |
| IIIB | 46-53% | 38% |
| IIIC | 28% | 15-25% |
| IV | 18% | 14% |

### Neuroblastoma

Neuroblastoma occurs in infants and young children and is rarely found in children older than 10 years. Neuroblastoma is by far the most common cancer in infants and the fourth most common type of cancer in children. There are approximately 650 new cases of neuroblastoma each year in the USA.

### Neuroblastoma Diagnosis

In about 90% of cases, neuroblastoma cells produce enough catecholamines to be detected by blood or urine tests. The 2 catecholamine metabolites most often measured are: homovanillic acid (HVA) and vanillylmandelic acid (VMA). Imaging tests such as a chest x-ray, computed tomography (CT), magnetic resonance imaging (MRI), ultrasound, positron emission tomography (PET), meta-iodobenzylguanidine (MIBG) scan and bone scan can also be performed. A biopsy is needed to confirm the diagnosis. A bone marrow aspiration and biopsy can be performed to see if the disease has spread to the bone marrow (which it does in about half of patients).

In as many as 6 or 7 of 10 cases, the disease is not diagnosed until it has already metastasised.

### Neuroblastoma Staging

Neuroblastoma is staged using the International Neuroblastoma Staging System (INSS)—Stage 1-Stage 4. Prognostic markers such as age, tumour grade, DNA ploidy, MYCN gene amplifications, cytogenetics, neurotrophin receptors and serum markers are used to calculate a child's chance of cure. These prognostic markers are combined with the stage of the disease to form three risk groups—low, intermediate and high. Low-risk children have a 5-year survival of around 95%. Intermediate-risk children's 5-year survival is around 85% to 90%. The 5-year survival of high-risk children is around 30%.

### Neuroblastoma Treatment

For children at low risk, surgery can often remove the entire tumour and bring about a complete cure. Chemotherapy is given if less than half the tumour can be surgically removed.

For children at intermediate risk, chemotherapy is usually given before or after surgery to control the disease. Chemotherapeutic agents used include cyclophosphamide, ifosfamide, cisplatin, carboplatin, vincristine, doxorubicin, etoposide, teniposide, topotecan. A second surgery or radiation therapy may also be required. Studies show that in some cases the use of radiation combined with chemotherapy produces better results than chemotherapy alone.

For children at high risk, when the cancer has spread too far to be completely removed by surgery, very intensive chemotherapy along with blood-forming stem cell transplant (bone marrow or peripheral blood) is used. Some experts estimate that this technique results in a cure in about 25% of children with neuroblastoma who would not be cured with the more standard treatments. Some recent studies offer hope that improved bone marrow transplant (BMT) or peripheral blood stem cell transplant (PBSC) methods may increase the rate to 30% to 60%. Surgery and/or radiation may be part of this treatment regimen. Radiation therapy can help relieve pain caused by advanced neuroblastoma. A highly radioactive form of MIBG is also being used for treating some patients with advanced neuroblastoma. Biologic agents such as 13-cis retinoic acid are often given for 6 months after therapy is completed to reduce the risk of recurrence.

### Osteosarcoma

Osteosarcoma is the most common bone cancer in children, adolescents and young adults (accounting for approximately 5% of childhood tumours) but it is still a rare disease with an annual incidence of 2-3 per million in the general population. There are about 900 new cases of osteosarcoma diagnosed in the United States each year (about 400 of which occur in children and adolescents younger than 20 years old), with approximately 300 deaths each year.

Osteosarcoma is a primary malignant tumour of the appendicular skeleton that is characterized by the direct formation of bone or osteoid tissue by the tumour cells. In children and adolescents, more than 50% of these tumours arise from the bones around the knee. Many people with osteosarcoma can be cured but not all and the price of cure even with the most modern treatments is high.

### Osteosarcoma Diagnosis

Diagnostic methods for osteosarcoma include an X-ray, bone scan, CT scan, PET scan and MRI of the affected area. A CT scan of the chest is also conducted to see if the cancer has spread to the lungs. Blood tests can be used to detect serum levels of alkaline phosphatase and/or LDH, which are increased in a considerable number of osteosarcoma patients, although serum levels do not correlate reliably with disease extent. The diagnosis of osteosarcoma must be verified histologically with a core needle biopsy or open biopsy. Micrometastatic disease is present at diagnosis in 80-90% of patients but undetectable with any of present tests.

### Osteosarcoma Staging

There are two staging systems for osteosarcoma: the Enneking system where low-grade tumours are stage I, high-grade tumours are stage II, and metastatic tumours (regardless of grade) are stage III and the American Joint Commission on Cancer (AJCC) system which stages osteosarcoma from IA to IVB.

There are essentially 2 categories of patients: those who present without clinically detectable metastatic disease (localized osteosarcoma) and the 15-20% of patients who present with clinically detectable metastatic disease (metastatic osteosarcoma). 85% to 90% of metastatic disease is in the lungs.

Osteosarcoma has one of the lowest survival rates for pediatric cancer. The overall 5-year survival rate for patients with non-metastatic osteosarcoma is over 70%. The 5-year survival rate for patients whose cancers have already metastasised at the time of their diagnosis is about 30%.

### Osteosarcoma Treatment

Once osteosarcoma has been diagnosed, the correct treatment needs to be selected. Successful treatment generally requires the combination of effective systemic chemotherapy and complete resection (amputation, limb preservation, or rotationplasty) of all clinically detectable disease (including resection of all overt metastatic disease). Protective weight bearing is recommended for patients with tumours of weight-bearing bones to prevent pathological fractures that could preclude limb-preserving surgery.

At least 80% of patients with localized osteosarcoma treated with surgery alone will develop metastatic disease. Randomized clinical trials have established that adjuvant chemotherapy is effective in preventing relapse or recurrence in patients with localized resectable primary tumours. The chemotherapeutic agents used include high-dose methotrexate, doxorubicin, cisplatin, high-dose ifosfamide, etoposide, carboplatin, cyclophosphamide, actinomycin D and bleomycin. Bone-seeking radioactive chemicals are sometimes used to treat osteosarcoma. Samarium-153 may be given in addition to external beam radiation therapy.

There is no difference in overall survival (OS) between patients initially treated by amputation and those treated with a limb-sparing procedure. In general, more than 80% of patients with extremity osteosarcoma can be treated by a limb-sparing operation and do not require amputation. Complications of limb-salvage surgery include infection and grafts or rods that become loose or broken. Limb-salvage surgery patients may need more surgery during the following 5 years, and some may eventually need an amputation. Limb length inequality is also a major potential problem for young children. Treatment options include extensible prostheses, amputation, and rotationplasty for these children.

Most recurrences of osteosarcoma develop within 2 to 3 years after treatment completion. Fewer than 30% of patients with localized resectable primary tumours treated with surgery alone can be expected to survive free of relapse. Recurrence of osteosarcoma is most often in the lung.

The ability to achieve a complete resection of recurrent disease is the most important prognostic factor at first relapse, with a 5-year survival rate of 20% to 45% following complete resection of metastatic pulmonary tumours and 20% following complete resection of metastases at other sites. Repeated resections of pulmonary recurrences can lead to extended disease control and possibly cure for some patients. Survival for patients with unresectable metastatic disease is less than 5%. Resection of metastatic disease followed by observation alone results in low overall and disease-free survival.

### Ovarian Cancer

Ovarian cancer accounts for about 1.9% of cancer cases globally and around 1.8% of deaths. Global incidence of ovarian cancer is around 205,000, predominantly in post-menopausal women in developed countries, with around 125,000 deaths. About 85% to 90% of ovarian cancers are epithelial ovarian carcinomas. About 5% of ovarian cancers are germ cell tumours and a smaller percentage are stromal tumours. Ovarian cancer is the eighth most common cancer among women. In the USA, about 20,200 new cases of ovarian cancer are diagnosed each year and it accounts for about 3% of all cancers in women. The risk of developing and dying from ovarian cancer is higher for white women than black women. Around two-thirds of women with ovarian cancer are 55 or older. Ovarian cancer ranks fifth in cancer deaths among women in the USA, accounting for more deaths than any other cancer of the female reproductive system. There are around 15,300 deaths in the USA from ovarian cancer each year. It has been estimated that approximately US$2.2 billion is spent in the USA each year on treating ovarian cancer.

### Ovarian Cancer Diagnosis

It is currently difficult to diagnose ovarian cancer at an early stage. Imaging tests such as ultrasound, computed tomography and magnetic resonance imaging can confirm whether a pelvic mass is present. Blood tests, including a CA-125 test and a laparoscopy are performed. Ovarian cancer is then confirmed by biopsy.

### Ovarian Cancer Staging

Ovarian cancer is staged using the American Joint Committee on Cancer (AJCC) TNM system—stage I-IV. The FIGO (International Federation of Gynecology and Obstetrics) system is also used. Ovarian cancers are also given a grade from 1-3. About 76% of women with ovarian cancer survive 1 year after diagnosis, and 45% survive longer than 5 years after diagnosis. If diagnosed and treated while the cancer has not spread outside the ovary, the 5-year survival rate is 94%. However, only 19% of all ovarian cancers are found at this early stage.

### Ovarian Cancer Treatment

Surgery for ovarian cancer includes hysterectomy, bilateral salpingectomy, bilateral oophorectomy and omentectomy. Debulking is performed in women in whom the cancer has spread widely throughout their abdomen.

Intraperitoneal (IP) chemotherapy using a combination therapy using a platinum compound, such as cisplatin or carboplatin, and a taxane, such as paclitaxel or docetaxel, is the standard approach. Tumour recurrence is sometimes treated with additional cycles of a platinum compound and/or a taxane. In other cases, recurrence is treated with other drugs, such as topotecan, anthracyclines such as doxorubicin (Adriamycin) and liposomal doxorubicin (Doxil), gemcitabine, cyclophosphamide, vinorelbine (Navelbine), hexamethylmelamine, ifosfamide, and etoposide. Resistance to currently-available chemotherapeutic agents is a major problem. Although complete clinical response is achieved in 75% of patients after initial treatment, most will develop recurrent disease and require re-treatment.

External beam radiation therapy can also sometimes be used.

### Ovarian Cancer 5-Year Survival by Stage:

For IA is 92.7%, for stage IB is 85.4%, for stage IC is 84.7%, for stage IIA is 78.6%, for stage IIB is 72.4%, for stage IIC is stage 64.4%, for stage IIIA is 50.8%, for stage IIIB is 42.4%, for stage IIIC is 31.5% and for stage IV is 17.5%.

### Pancreatic Cancer

Pancreatic cancer is a very difficult cancer to detect and the prognosis for patients is usually very poor. The number of new cases and deaths per year is almost equal. Global incidence of pancreatic cancer is approximately 230,000 cases (about 2% of all cancer cases), with about 225,000 deaths (3.4% of cancer deaths) per year. It is much more prevalent in the developed world. In the USA, there are about 34,000 new cases per year, with about 32,000 deaths. It has been estimated that approximately US$1.5 billion is spent in the USA each year on treating pancreatic cancer.

### Pancreatic Cancer Diagnosis

Pancreatic cancer is very difficult to detect and very few pancreatic cancers are found early. Patients usually have no symptoms until the cancer has spread to other organs. There are currently no blood tests or easily available screening tests that can accurately detect early cancers of the pancreas. An endoscopic ultrasound followed by a biopsy is the best way to diagnose pancreatic cancer. Other detection methods include CT, CT-guided needle biopsy, PET, ultrasonography and MRI. Blood levels of CA 19-9 and carcinoembryonic antigen (CEA) may be elevated but by the time blood levels are high enough to be detected, the cancer is no longer in its early stages.

### Pancreatic Cancer Staging

Pancreatic cancer has four stages, stage Ito stage IV according to the American Joint Committee on Cancer (AJCC) TNM system. Pancreatic cancer is also divided into resectable, locally advanced (unresectable) and metastatic cancer. For patients with advanced cancers, the overall survival rate is <1% at 5 years with most patients dying within 1 year.

### Pancreatic Cancer Treatment

Surgery is the only method of curing pancreatic cancer. About 10% of pancreatic cancers are contained entirely within the pancreas at the time of diagnosis and attempts to remove the entire cancer by surgery may be successful in some of these patients. The 5-year survival for those undergoing surgery with the intent of completely removing the cancer is about 20%. Potentially curative surgery, usually by pancreaticoduodenectomy (Whipple procedure), is used when it may be possible to remove all of the cancer. Palliative surgery may be performed if the tumour is too widespread to be completely removed. Removing only part of the cancer does not allow patients to live longer. Pancreatic cancer surgery is difficult to perform with a high likelihood of complications.

External beam radiation therapy combined with chemotherapy can be given before or after surgery and can also be given to patients whose tumours are too widespread to be removed by surgery. The main chemotherapeutic agents which are used are gemcitabine and 5-fluorouracil. Targeted therapy using drugs such as erlotinib and cetuximab may be of benefit to patients with advanced pancreatic cancer.

### Prostate Cancer

Prostate cancer is the third most common cancer in the world amongst men and it accounts for 5.4% of all cancer cases globally and 3.3% of cancer-related deaths. Global incidence of prostate cancer is around 680,000 cases, with about 221,000 deaths. In the USA, prostate cancer is the most common cancer, other than skin cancers, in American men. About 234,460 new cases of prostate cancer are diagnosed in the USA each year. About 1 man in 6 will be diagnosed with prostate cancer during his lifetime, but only 1 in 34 will die of it. A little over 1.8 million men in the USA are survivors of prostate cancer. The risk of developing prostate cancer rises significantly with age and 60% of cases occur in men over the age of 70. Prostate cancer is the second leading cause of cancer death in American men. Around 27,350 men in the USA die of prostate cancer each year. Prostate cancer accounts for about 10% of cancer-related deaths in men. Modern methods of detection and treatment mean that prostate cancers are now found earlier and treated more effectively. This has led to a yearly drop in death rates of about 3.5% in recent years. Prostate cancer is most common in North America and northwestern Europe. It is less common in Asia, Africa, Central America, and South America. It has been estimated that approximately US$8.0 billion is spent in the USA each year on treating prostate cancer.

### Prostate Cancer Diagnosis

Prostate cancer can often be found early by testing the amount of prostate-specific antigen (PSA) in the blood. A digital rectal exam (DRE) can also be performed. However, there are potential problems with the current screening methods. Neither the PSA test nor the DRE is 100% accurate. A core needle biopsy is the main method used to diagnose prostate cancer. A transrectal ultrasound (TRUS) may be used during a prostate biopsy.

### Prostate Cancer Staging

Prostate cancers are graded according to the Gleason system, graded from 1-5, which results in the Gleason score, from 1-10. Prostate cancer is staged using the American Joint Committee on Cancer (AJCC) TNM system and combined with the Gleason score to give stages from I-IV. Ninety one percent of all prostate cancers are found in the local and regional stages; the 5-year relative survival rate for these men is nearly 100%. The 5-year relative survival rate for men whose prostate cancers have already spread to distant parts of the body at the time of diagnosis is about 34%.

### Prostate Cancer Treatment

Because prostate cancer often grows very slowly, some men never have treatment and expectant management is recommended. If treatment is required and the cancer is not thought to have spread outside of the gland, a radical prostatectomy can be performed. Transurethral resection of the prostate (TURP) can be performed to relieve symptoms but not to cure prostate cancer. External beam radiation therapy (three-dimensional conformal radiation therapy (3DCRT), intensity modulated radiation therapy (IMRT) or conformal proton beam radiation therapy) or brachytherapy can also be used as treatment.

Cryosurgery is sometimes used to treat localized prostate cancer but as not much is known about the long-term effectiveness of cryosurgery, it is not routinely used as a first treatment for prostate cancer. It can be used for recurrent cancer after other treatments.

Androgen deprivation therapy (ADT) (orchiectomy or luteinizing hormone-releasing hormone (LHRH) analogs or antagonists) can be used to shrink prostate cancers or make them grow more slowly.

Chemotherapy is sometimes used if prostate cancer has spread outside of the prostate gland and is hormone therapy resistant. Chemotherapeutic agents include docetaxel, prednisone, doxorubicin, etoposide, vinblastine, paclitaxel, carboplatin, estramustine, vinorelbine. Like hormone therapy, chemotherapy is unlikely to result in a cure.

### Renal Cell Cancer

The incidence of kidney cancer is much higher in developed countries, being the sixth most common form of cancer in Western Europe. Kidney cancer accounts for about 1.9% of cancer cases globally and 1.5% of deaths. Global incidence of kidney cancer is around 208,000 cases, with over 100,000 deaths. Around 38,900 new cases of kidney cancer are diagnosed in the USA each year, with around 12,800 deaths. It is very uncommon under age 45, and its incidence is highest between the ages of 55 and 84. The rate of people developing kidney cancer has been increasing at about 1.5% per year but the death rate has not been increasing. Renal cell carcinoma accounts for more than 90% of malignant kidney tumours. It has been estimated that approximately US$1.9 billion is spent in the USA each year on treating kidney cancer.

### Renal Cell Cancer Diagnosis

Many renal cell cancers are found at a late stage; they can become quite large without causing any pain or discomfort. Because the kidney is deep inside the body, small renal cell tumours cannot be seen or felt during a physical exam. There are no simple tests that can detect renal cell cancer early. About 25% of patients with renal cell carcinoma will already have metastatic spread of their cancer when they are diagnosed. Imaging tests such as computed tomography (CT) scans and magnetic resonance imaging (MRI) can find small renal cell carcinomas. However, these imaging tests are relatively expensive and cannot always distinguish benign tumours from small renal cell carcinomas.

Renal cell cancer can often be diagnosed without the need for a biopsy using a CT scan, MRI, ultrasound, positron emission tomography (PET) scan, intravenous pyelogram (IVP) and/or angiography. Fine needle aspiration biopsy may however be valuable when imaging results are not conclusive enough to warrant removing a kidney.

### Renal Cell Cancer Staging

Renal cell cancers are usually graded on a scale of 1-4. Renal cell cancer is also staged using the American Joint Committee on Cancer (AJCC) TNM system—stage I-IV. The University of California Los Angeles Integrated Staging System can also be used, which divides patients without any tumour spread into three groups—low risk, intermediate risk and high risk. The 5-year cancer-specific survival for the low-risk group is 91%, for the intermediate-risk group is 80%, and for the high-risk group is 55%. Patients with tumour spread are also divided into three groups—low, intermediate and high risk. The 5-year cancer-specific survival for the low-risk group is 32%, for the intermediate-risk group 20% and for the high-risk group 0%.

### Renal Cell Cancer Treatment

Surgery by radical nephrectomy (and sometimes regional lymphadenectomy), partial nephrectomy or laparoscopic nephrectomy is the main treatment for renal cell carcinoma. External beam radiation therapy is sometimes used as the main treatment for renal cell cancer if a person's general health is too poor to undergo surgery. Radiation therapy can also be used to palliate symptoms of renal cell cancer. Unfortunately, renal cell carcinomas are not very sensitive to radiation. Using radiation therapy before or after removing the cancer is not routinely recommended because studies have shown no improvement in survival rates. Renal cell cancers are very resistant to present forms of chemotherapy, and there is no standard way to treat it with drugs. Some drugs, such as vinblastine, floxuridine, and 5-fluorouracil (5-FU) are mildly effective. A combination of 5-FU and gemcitabine has benefited some patients. A 5-FU-like drug, capecitabine, may also have some benefit.

Cytokines (interleukin-2 (IL-2) and interferon-alpha) have become one of the standard treatments for metastatic renal cell carcinoma. These cause the cancers to shrink to less than half their original size in about 10% to 20% of patients. Patients who respond to IL-2 tend to have lasting responses. Recent research with a combination of IL-2, interferon, and chemotherapy (using 5-fluorouracil) is also promising and may offer a better chance of partial or complete remission. Cytokine therapy does have severe side affects however.

Sorafenib (Nexavar) has been shown to slow the progression of the cancer in patients with advanced disease. It acts by blocking both angiogenesis and growth-stimulating molecules in the cancer cell. Sunitinib (Sutent) is another drug that attacks both blood vessel growth and other targets that stimulate cancer cell growth. Promising results have also been seen in studies of this drug with tumours shrinking in about one-third of patients and tumours staying about the same size in another third. Bevacizumab (Avastin) is an angiogenesis inhibitor. This drug is already approved for use against other cancer types and recent studies have shown it may also be effective against renal cell cancer.

### Renal Cell Cancer Survival by Stage

| T stage cancer | 5/10-year cancer-specific survival |
|---|---|
| T1 | 95%/91% |
| T2 | 80%/70% |
| T3a | 66%/53% |
| T3b | 52%/43% |
| T3c | 43%/42% |

### Retinoblastoma

Retinoblastoma is the only common type of eye cancer in children. About 4 out of every million children under age 15, or about 250 children, are diagnosed with retinoblastoma each year in the USA. The rate is higher in infants and very young children. About 60% to 75% of children with retinoblastoma have a tumour in only one eye. In about 25% to 40% of cases, both eyes are affected. When both eyes are affected, it is always caused by a gene mutation present at birth.

### Retinoblastoma Diagnosis

Most retinoblastomas are found and treated before they have spread outside the globe of the eye. The most important gene involved in retinoblastoma is the tumour suppressor gene known as Rb. One third of retinoblastoma patients have hereditary retinoblastoma with a germline mutation of the Rb gene which can be detected by testing DNA of blood cells. In the remaining two thirds of retinoblastoma patients, the Rb gene mutation happens during their early life and occurs only in one cell in one eye. An ophthalmologist can diagnose retinoblastoma by examining the retina. Imaging tests such as ultrasound and magnetic resonance imaging (MRI) can also be used.

### Retinoblastoma Staging

The Reese-Ellsworth staging system is used to classify cases of retinoblastoma that have not spread beyond the eye into 5 groups. Well over 90% of children with retinoblastoma that has not spread beyond the eye are cured. The major challenge is preserving their vision and the groups help to determine the likelihood of preserving vision and controlling the tumour. Staging is sometimes based upon the ABC Classification—groups A-E.

### Retinoblastoma Treatment

When tumour occurs in only one eye, it tends to get quite large before it is diagnosed. Vision has often already been destroyed, with no hope of restoration. The usual treatment in this case is enucleation. When retinoblastoma occurs in both eyes, enucleation of both eyes would automatically result in complete blindness. This is the safest treatment if neither eye has useful vision but if there is any chance of preserving useful vision in one or both eyes by using more conservative treatments, these are considered.

Radiation therapy (external beam radiation therapy or brachytherapy), compared with surgery, has the advantage of possibly preserving vision in the eye. Unfortunately, radiation also has several potential disadvantages. Radiation therapy can damage surrounding normal tissue, which may eventually lead to cataracts and damage to the retina, which would reduce vision. Photocoagulation, thermotherapy or cryotherapy can be used as treatment for small tumours. Chemoreduction may be used to shrink small tumours, which can then be treated more effectively by photocoagulation, cryotherapy, thermotherapy, or brachytherapy to completely kill the tumour.

Chemotherapy is often used to treat children whose retinoblastoma has spread beyond the eye. Unfortunately, retinoblastomas tend to become resistant to chemotherapy. Retinoblastoma metastases often shrink for a period of time, but usually start growing again within a year. Chemotherapeutic agents used include carboplatin, cisplatin, vincristine, etoposide, teniposide, cyclophosphamide and doxorubicin.

A group 1 retinoblastoma is very likely to be controlled with chemotherapy, photocoagulation, cryotherapy, thermotherapy, brachytherapy, or external beam radiation therapy while still preserving vision in the eye.

A group 4 or especially group 5 retinoblastoma is very unlikely to be controlled with chemotherapy or radiation therapy, and even if it were, the vision in the eye would be very poor. Enucleation is usually performed in these cases.

When the cancer has spread outside of the orbit to the bones and bone marrow, the chances of cure using conventional chemotherapy and surgery are very poor; in these cases, the use of higher doses of chemotherapy with an autologous stem cell transplant can be successful, and more than 50% of the children can be cured. When the cancer has spread to the brain, the chances of cure even using stem cell transplant are very low.

### Therapeutic Challenges

The major challenges in treatment of the above mentioned cancers are to improve early detection rates, to find new non-invasive markers that can be used to follow disease progression and identify relapse, and to find improved and less toxic therapies, especially for more advanced disease where 5 year survival is still poor. There is a great need to identify targets which are more specific to the cancer cells, e.g. ones which are expressed on the surface of the tumour cells so that they can be attacked by promising new approaches like immunotherapeutics and targeted toxins.

### SUMMARY OF THE INVENTION

The present invention provides methods and compositions for:
therapy of, or
drug development for, or
screening, diagnosis and prognosis for, or
monitoring the effectiveness of treatment for
   B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma (including patient stratification).

We have used mass spectrometry to identify peptides generated by gel electrophoresis or tagging with iTRAQ reagents and tryptic digest of membrane proteins extracted from lymphoid, breast, cervical, colorectal, gastric, brain, liver, lung, skin, neuronal, osteoblast, ovarian, pancreatic, prostate, kidney and eye cancer tissue samples. Peptide sequences were compared to existing protein and cDNA databases and the corresponding gene sequences identified. The proteins of the invention have not been previously reported to originate from lymphocytic, breast, cervical, colorectal, gastric, brain, liver, lung, skin, neuronal, osteoblast, ovarian, pancreatic, prostate, kidney or eye cancer cell membranes and represent proteins of new therapeutic and/or diagnostic value.

Thus, a first aspect of the invention provides methods of treating B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma, comprising administering to a patient a therapeutically effective amount of a compound that modulates (e.g., upregulates or downregulates) or complements the expression or the biological activity (or both) of one or more of the proteins of the invention in patients having B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma, in order to ((a) prevent the onset or development of B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma; (b) prevent the progression of B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma; or (c) ameliorate the symptoms of B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma.

Thus according to a second aspect of the invention we provide a method of detecting, diagnosing and/or screening for or monitoring the progression of B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma or of monitoring the effect of an anti-B-cell non-Hodgkin's lymphoma, anti-breast cancer, anti-cervical cancer, anti-colorectal cancer, anti-gastric cancer, anti-glioblastoma, anti-hepatocellular carcinoma, anti-lung cancer, anti-lymphoid leukaemia (particularly acute T-cell leukaemia and chronic lymphocytic leukaemia), anti-melanoma, anti-neuroblastoma, anti-osteosarcoma, anti-ovarian cancer, anti-pancreatic cancer, anti-prostate cancer, anti-renal cell cancer or anti-retinoblastoma drug or therapy in a subject which comprises detecting the presence or level of the proteins of the invention, or one or more fragments thereof, or the presence or level of nucleic acid encoding the proteins of the invention or the presence or level of the activity of the proteins of the invention or which comprises detecting a change in the level thereof in said subject.

According to a third aspect of the invention we provide a method of detecting, diagnosing and/or screening for B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma in a candidate subject which comprises detecting the presence of the proteins of the invention, or one or more fragments thereof, or the presence of nucleic acid encoding the proteins of the invention or the presence of the activity of the proteins of the invention in said candidate subject, in which either (a) the presence of an elevated level of the proteins of the invention or said one or more fragments thereof or an elevated level of nucleic acid encoding the proteins of the invention or the presence of an elevated level of the activity of the proteins of the invention in the candidate subject as compared with the level in a healthy subject or (b) the presence of a detectable level of the proteins of the invention or said one or more fragments thereof or a detectable level of nucleic acid encoding the proteins of the invention or the presence of a detectable level of the activity of the proteins of the invention in the candidate subject as compared with a corresponding undetectable level in a healthy subject indicates the presence of B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma in said subject.

According to a fourth aspect of the invention we provide a method of monitoring the progression of B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma in a subject or of monitoring the effect of an anti-B-cell non-Hodgkin's lymphoma, anti-breast cancer, anti-cervical cancer, anti-colorectal cancer, anti-gastric cancer, anti-glioblastoma, anti-hepatocellular carcinoma, anti-lung cancer, anti-lymphoid leukaemia (particularly acute T-cell leukaemia and chronic lymphocytic leukaemia), anti-melanoma, anti-neuroblastoma, anti-osteosarcoma, anti-ovarian cancer, anti-pancreatic cancer, anti-prostate cancer, anti-renal cell cancer or anti-retinoblastoma drug or therapy which comprises detecting the presence of the proteins of the invention, or one or more fragments thereof, or the presence of nucleic acid encoding the proteins of the invention or the presence of the activity of the proteins of the invention in said candidate subject at a first time point and at a later time point, the presence of an elevated or lowered level of the proteins of the invention or said one or more fragments thereof or an elevated or lowered level of nucleic acid encoding the proteins of the invention or the presence of an elevated or lowered level of the activity of the proteins of the invention in the subject at the later time point as compared with the level in the subject at said first time point, indicating the progression or regression of B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma or indicating the effect or non-effect of an anti-B-cell non-Hodgkin's lymphoma, anti-breast cancer, anti-cervical cancer, anti-colorectal cancer, anti-gastric cancer, anti-glioblastoma, anti-hepatocellular carcinoma, anti-lung cancer, anti-lymphoid leukaemia (particularly acute T-cell leukaemia and chronic lymphocytic leukaemia), anti-melanoma, anti-neuroblastoma, anti-osteosarcoma, anti-ovarian cancer, anti-pancreatic cancer, anti-prostate cancer, anti-renal cell cancer or anti-retinoblastoma drug or therapy in said subject.

The presence of the proteins of the invention, or one or more fragments thereof, or the presence of nucleic acid encoding the proteins of the invention or the presence of the activity of the proteins of the invention may, for example, be detected by analysis of a biological sample obtained from said subject.

The method of invention may typically include the step of obtaining a biological sample for analysis from said subject. In one or more aspects the methods of the invention do not include the step of obtaining a biological sample from a subject.

In one aspect of the invention provides monoclonal and polyclonal antibodies or other affinity reagents such as an Affibodies capable of immunospecific binding to the proteins of the invention and compositions comprising same, for example for use in treatment or prophylaxis, such for the treatment or prophylaxis of cancer, particularly a cancer described herein.

The biological sample used can be from any source such as a serum sample or a tissue sample, e.g. lymphoid, breast, cervical, colorectal, gastric, brain, liver, lung, skin, neuronal, osteoblast, ovarian, pancreatic, prostate, kidney or eye tissue. For instance, when looking for evidence of metastatic breast cancer, cervical cancer, colorectal cancer, gastric cancer, hepatocellular carcinoma, lung cancer, melanoma, neuroblastoma, osteosarcoma, ovarian cancer, pancreatic cancer, prostate cancer, renal cell cancer and retinoblastoma, one would look at major sites of breast cancer, cervical cancer, colorectal cancer, gastric cancer, hepatocellular carcinoma, lung cancer, melanoma, neuroblastoma, osteosarcoma, ovarian cancer, pancreatic cancer, prostate cancer, renal cell cancer and retinoblastoma metastasis, e.g. the liver, the lungs and bones for breast cancer; the bladder and the rectum for cervical cancer; the liver, the peritoneal cavity, the pelvis, the retroperitoneum and the lungs for colorectal cancer; the liver, the lungs, the brain and bones for gastric cancer; the lungs and bones for hepatocellular carcinoma; the brain, the liver, the bones and adrenal glands for lung cancer; the lungs, the brain and bones for melanoma; the liver and bones for neuroblastoma; the lungs and other bones for osteosarcoma; the abdomen for ovarian cancer; the liver for pancreatic cancer; the bladder, the rectum and bones for prostate cancer; the lungs, the liver and bones for renal cell cancer and the brain and bones for retinoblastoma.

Alternatively the presence of the proteins of the invention, or one or more fragments thereof, or the presence of nucleic acid encoding the proteins of the invention or the presence of the activity of the proteins of the invention may be detected by analysis in situ.

In certain embodiments, methods of diagnosis described herein may be at least partly, or wholly, performed in vitro.

Suitably the presence of the proteins of the invention, or one or more fragments thereof, or the presence of nucleic acid encoding the proteins of the invention or the presence of the activity of the proteins of the invention is detected quantitatively.

For example, quantitatively detecting may comprise:
(a) contacting a biological sample with an affinity reagent that is specific for the proteins of the invention, said affinity reagent optionally being conjugated to a detectable label; and
(b) detecting whether binding has occurred between the affinity reagent and at least one species in the sample, said detection being performed either directly or indirectly.

Alternatively the presence of the proteins of the invention, or one or more fragments thereof, or the presence of nucleic acid encoding the proteins of the invention or the presence of the activity of the proteins of the invention may be detected quantitatively by means involving use of an imaging technology.

In another embodiment, the method of the invention involves use of immunohistochemistry on tissue sections in order to determine the presence of the proteins of the invention, or one or more fragments thereof, or the presence of nucleic acid encoding the proteins of the invention or the presence of the activity of the proteins of the invention, and thereby to localise B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma cells.

In one embodiment the presence of the proteins of the invention or one or more epitope-containing fragments thereof is detected, for example using an affinity reagent capable of specific binding to the proteins of the invention or one or more fragments thereof, such as an antibody.

In another embodiment the activity of the proteins of the invention is detected.

According to another aspect of the invention there is provided a method of detecting, diagnosing and/or screening for or monitoring the progression of B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma or of monitoring the effect of an anti-B-cell non-Hodgkin's lymphoma, anti-breast cancer, anti-cervical cancer, anti-colorectal cancer, anti-gastric cancer, anti-glioblastoma, anti-hepatocellular carcinoma, anti-lung cancer, anti-lymphoid leukaemia (particularly acute T-cell leukaemia and chronic lymphocytic leukaemia), anti-melanoma, anti-neuroblastoma, anti-osteosarcoma, anti-ovarian cancer, anti-pancreatic cancer, anti-prostate cancer, anti-renal cell cancer or anti-retinoblastoma drug or therapy in a subject which comprises detecting the presence or level of antibodies capable of immunospecific binding to the proteins of the invention, or one or more epitope-containing fragments thereof or which comprises detecting a change in the level thereof in said subject.

According to another aspect of the invention there is also provided a method of detecting, diagnosing and/or screening for B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma in a subject which comprises detecting the presence of antibodies capable of immunospecific binding to the proteins of the invention, or one or more epitope-containing fragments thereof in said subject, in which (a) the presence of an elevated level of antibodies capable of immunospecific binding to the proteins of the invention or said one or more epitope-containing fragments thereof in said subject as compared with the level in a healthy subject or (b) the presence of a detectable level of antibodies capable of immunospecific binding to the proteins of the invention or said one or more epitope-containing fragments thereof in said subject as compared with a corresponding undetectable level in a healthy subject indicates the presence of B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma in said subject.

One particular method of detecting, diagnosing and/or screening for B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma comprises:
(a) bringing into contact with a biological sample to be tested the proteins of the invention, or one or more epitope-containing fragments thereof; and
(b) detecting the presence of antibodies in the subject capable of immunospecific binding to the proteins of the invention, or one or more epitope-containing fragments thereof.

According to another aspect of the invention there is provided a method of monitoring the progression of B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma or of monitoring the effect of an anti-B-cell non-Hodgkin's lymphoma, anti-breast cancer, anti-cervical cancer, anti-colorectal cancer, anti-gastric cancer, anti-glioblastoma, anti-hepatocellular carcinoma, anti-lung cancer, anti-lymphoid leukaemia (particularly acute T-cell leukaemia and chronic lymphocytic leukaemia), anti-melanoma, anti-neuroblastoma, anti-osteosarcoma, anti-ovarian cancer, anti-pancreatic cancer, anti-prostate cancer, anti-renal cell cancer or anti-retinoblastoma drug or therapy in a subject which comprises detecting the presence of antibodies capable of immunospecific binding to the proteins of the invention, or one or more epitope-containing fragments thereof in said subject at a first time point and at a later time point, the presence of an elevated or lowered level of antibodies capable of immunospecific binding to the proteins of the invention, or one or more epitope-containing fragments thereof in said subject at the later time point as compared with the level in said subject at said first time point, indicating the progression or regression of B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma or the effect or non-effect of an anti-B-cell non-Hodgkin's lymphoma, anti-breast cancer, anti-cervical cancer, anti-colorectal cancer, anti-gastric cancer, anti-glioblastoma, anti-hepatocellular carcinoma, anti-lung cancer, anti-lymphoid leukaemia (particularly acute T-cell leukaemia and chronic lymphocytic leukaemia), anti-melanoma, anti-neuroblastoma, anti-osteosarcoma, anti-ovarian cancer, anti-pancreatic cancer, anti-prostate cancer, anti-renal cell cancer or anti-retinoblastoma drug or therapy in said subject.

The presence of antibodies capable of immunospecific binding to the proteins of the invention, or one or more epitope-containing fragments thereof is typically detected by analysis of a biological sample obtained from said subject (exemplary biological samples are mentioned above, e.g. the sample is a sample of lymphoid, breast, cervical, colorectal, gastric, brain, liver, lung, skin, neuronal, osteoblast, ovarian, pancreatic, prostate, kidney or eye tissue, or else a sample of blood or saliva).

The method typically includes the step of obtaining said biological sample for analysis from said subject.

The antibodies that may be detected include IgA, IgM and IgG antibodies.

In any of the above methods, the level that may be detected in the candidate subject who has B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma is 2 or more fold higher than the level in the healthy subject.

Another aspect of the invention is an agent capable of specific binding to the proteins of the invention, or a fragment thereof, or a hybridising agent capable of hybridizing to nucleic acid encoding the proteins of the invention or an agent capable of detecting the activity of the proteins of the invention for use in screening for, detecting and/or diagnosing disease, such as cancer, and especially B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma.

Another aspect of the invention is the proteins of the invention, or fragments thereof for use in screening for, detecting and/or diagnosing disease such as cancer, and especially B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma.

Another aspect of the invention is affinity reagents capable of specific binding to the proteins of the invention or fragments thereof, for example an affinity reagent which contains or is conjugated to a detectable label or contains or is conjugated to a therapeutic moiety such as a cytotoxic moiety. The affinity reagent may, for example, be an antibody.

Another aspect of the invention is hybridizing agents capable of hybridizing to nucleic acid encoding the proteins of the invention, for example, a hybridizing agent which contains or is conjugated to a detectable label. One example of a hybridizing agent is an inhibitory RNA (RNAi). Other examples include anti-sense oligonucleotides and ribozymes.

The invention also provides kits containing the proteins of the invention and/or one or more fragments thereof or containing one or more aforementioned affinity reagents and/or hybridizing agents or containing one or more agents capable of detecting the activity of the proteins of the invention together with instructions for their use in an aforementioned method. The kit may further contain reagents capable of detecting and reporting the binding of said affinity reagents and/or hybridizing agents to their binding partners.

Another aspect of the invention is pharmaceutical compositions comprising a therapeutically effective amount of affinity reagents capable of specific binding to the proteins of the invention or a fragment thereof.

Another aspect of the invention is a pharmaceutically acceptable diluent or carrier and a pharmaceutical composition comprising one or more affinity reagents or hybridizing reagents as aforesaid and a pharmaceutically acceptable diluent or carrier.

In one embodiment the cancer to be detected, prevented or treated is B-cell non-Hodgkin's lymphoma.

In one embodiment the cancer to be detected, prevented or treated is breast cancer.

In one embodiment the cancer to be detected, prevented or treated is cervical cancer.

In one embodiment the cancer to be detected, prevented or treated is colorectal cancer.

In one embodiment the cancer to be detected, prevented or treated is gastric cancer.

In one embodiment the cancer to be detected, prevented or treated is glioblastoma.

In one embodiment the cancer to be detected, prevented or treated is hepatocellular carcinoma.

In one embodiment the cancer to be detected, prevented or treated is lung cancer.

In one embodiment the cancer to be detected, prevented or treated is lymphoid leukaemia (particularly acute T-cell leukaemia and chronic lymphocytic leukaemia).

In one embodiment the cancer to be detected, prevented or treated is melanoma.

In one embodiment the cancer to be detected, prevented or treated is neuroblastoma.

In one embodiment the cancer to be detected, prevented or treated is osteosarcoma.

In another embodiment the cancer to be detected, prevented or treated is ovarian cancer.

In another embodiment the cancer to be detected, prevented or treated is pancreatic cancer.

In another embodiment the cancer to be detected, prevented or treated is prostate cancer.

In another embodiment the cancer to be detected, prevented or treated is renal cell cancer.

In another embodiment the cancer to be detected, prevented or treated is retinoblastoma.

In another aspect, a method for the treatment or prophylaxis of cancer is presented, wherein OGTA002, OGTA009, OGTA016, OGTA028, OGTA037, OGTA041, OGTA053, OGTA054, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA222, OGTA236, OGTA237, OGTA247, OGTA248, OGTA249, OGTA257 or OGTA271 is expressed in said cancer, which comprises administering to a subject in need thereof a therapeutically effective amount of an affinity reagent which binds to OGTA002, OGTA009, OGTA016, OGTA028, OGTA037, OGTA041, OGTA053, OGTA054, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA222, OGTA236, OGTA237, OGTA247, OGTA248, OGTA249, OGTA257 or OGTA271. In an embodiment thereof, the method is directed to the treatment or prophylaxis of a cancer selected from the group consisting of B-cell non-Hodgkin's lymphoma, breast cancer, cervical cancer, colorectal cancer, gastric cancer, glioblastoma, hepatocellular carcinoma, lung cancer, lymphoid leukaemia, acute T-cell leukaemia, chronic lymphocytic leukaemia, melanoma, neuroblastoma, osteosarcoma, ovarian cancer, pancreatic cancer, prostate cancer, renal cell cancer and retinoblastoma, or has increased likelihood of developing B-cell non-Hodgkin's lymphoma, breast cancer, cervical cancer, colorectal cancer, gastric cancer, glioblastoma, hepatocellular carcinoma, lung cancer, lymphoid leukaemia, acute T-cell leukaemia, chronic lymphocytic leukaemia, melanoma, neuroblastoma, osteosarcoma, ovarian cancer, pancreatic cancer, prostate cancer, renal cell cancer or retinoblastoma.

In a particular embodiment thereof, the affinity reagent comprises a label. In a more particular embodiment, the label is a detectable label or therapeutic moiety. Even more particularly, the therapeutic moiety is selected from the group consisting of a cytotoxic moiety and a radioactive isotype.

In another particular embodiment, the affinity reagent is selected from the group consisting of fusion proteins and antibodies. In a more particular embodiment, the antibody is a monoclonal antibody, a humanized antibody, a bispecific antibody, a non-fucosylated antibody, an antibody fragment, or an antibody mimetic. In a particular embodiment thereof, the affinity reagent has cytotoxicity against OGTA002, OGTA009, OGTA016, OGTA028, OGTA037, OGTA041, OGTA053, OGTA054, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA222, OGTA236, OGTA237, OGTA247, OGTA248, OGTA249, OGTA257 or OGTA271 antigen expressing cells in the presence of human complement or in the presence of human immune effector cells.

Also encompassed herein is a method for screening for or diagnosis of, or for monitoring or assessing treatment of B-cell non-Hodgkin's lymphoma, breast cancer, cervical cancer, colorectal cancer, gastric cancer, glioblastoma, hepatocellular carcinoma, lung cancer, lymphoid leukaemia, acute T-cell leukaemia, chronic lymphocytic leukaemia, melanoma, neuroblastoma, osteosarcoma, ovarian cancer, pancreatic cancer, prostate cancer, renal cell cancer or retinoblastoma in a human subject, which comprises analyzing a sample from a patient for: the presence or absence; or the quantity; of OGTA002, OGTA009, OGTA016, OGTA028, OGTA037, OGTA041, OGTA053, OGTA054, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA222, OGTA236, OGTA237, OGTA247, OGTA248, OGTA249, OGTA257 or OGTA271, or a fragment thereof, whereby an increase in the level of OGTA002, OGTA009, OGTA016, OGTA028, OGTA037, OGTA041, OGTA053, OGTA054, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA222, OGTA236, OGTA237, OGTA247, OGTA248, OGTA249, OGTA257 or OGTA271, or a fragment thereof relative to a healthy control is indicative of the presence of or increased likelihood of developing B-cell non-Hodgkin's lymphoma, breast cancer, cervical cancer, colorectal cancer, gastric cancer, glioblastoma, hepatocellular carcinoma, lung cancer, lymphoid leukaemia, acute T-cell leukaemia, chronic lymphocytic leukaemia), melanoma, neuroblastoma, osteosarcoma, ovarian cancer, pancreatic cancer, prostate cancer, renal cell cancer or retinoblastoma.

In a particular embodiment thereof, the method comprises contacting said sample with one or more anti-:OGTA002, OGTA009, OGTA016, OGTA028, OGTA037, OGTA041, OGTA053, OGTA054, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA222, OGTA236, OGTA237, OGTA247, OGTA248, OGTA249, OGTA257 or OGTA271 affinity reagents capable of specific binding to OGTA002, OGTA009, OGTA016, OGTA028, OGTA037, OGTA041, OGTA053, OGTA054, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA222, OGTA236, OGTA237, OGTA247, OGTA248, OGTA249, OGTA257 or OGTA271 as defined in SEQ ID Nos: 1-48 respectively or a fragment or derivative thereof. In a more particular embodiment, the analyzing comprises imaging said sample. In a still more particular embodiment, the affinity reagent comprises a detectable label. In another particular embodiment, the method comprises detecting OGTA002, OGTA009, OGTA016, OGTA028, OGTA037, OGTA041, OGTA053, OGTA054, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA222, OGTA236, OGTA237, OGTA247, OGTA248, OGTA249, OGTA257 or OGTA271 mRNA.

Other aspects of the present invention are set out below and in the claims herein.

### BRIEF DESCRIPTION OF THE FIGURES

FIG. 1 shows the amino acid sequences of the proteins of the invention. The tryptics detected experimentally by mass spectrometry are highlighted—mass match peptides are shown in bold, tandem peptides are underlined.

FIGS. 2 and 4 show so-called Box Plot data for OGTA066, as described in Examples 3 and 4.

FIG. 3 shows ROC curve data for OGTA066, as described in Example 4.

FIG. 5 shows a histogram plot depicting internalization of anti-CDH3 monoclonal antibodies by A431 cells, as compared to the anti-human IgG isotype control antibody. See also Example 7.

FIG. 6 depicts results of flow cytometry analyses, which revealed that the anti-MUC13 monoclonal antibodies bound effectively to the cell-surface human MUC13 expressed on HT-29 and LS174T cells. See also Example 10.

### DETAILED DESCRIPTION OF THE INVENTION

The invention encompasses the administration of therapeutic compositions to a mammalian subject to treat or prevent B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma.

The invention also encompasses clinical screening, diagnosis and prognosis in a mammalian subject for identifying patients most likely to respond to a particular therapeutic treatment, for monitoring the results of B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma therapy, for drug screening and drug development. The mammalian subject may be a non-human mammal, for example a human, such as a human adult, i.e. a human subject at least 21 (more preferably at least 35, at least 50, at least 60, at least 70, or at least 80) years old. For clarity of disclosure, and not by way of limitation, the invention will be described with respect to the analysis of lymphoid, breast, cervical, colorectal, gastric, brain, liver, lung, skin, neuronal, osteoblast, ovarian, pancreatic, prostate, kidney and eye tissue. However, as one skilled in the art will appreciate, the assays and techniques described below can be applied to other types of patient samples, including body fluids (e.g. blood, urine or saliva), a tissue sample from a patient at risk of having B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma (e.g. a biopsy such as a bone marrow, breast, cervical, liver, stomach, brain, lung, skin, bone, ovarian, pancreatic, prostate or kidney biopsy) or homogenate thereof.

The methods and compositions of the present invention are specially suited for screening, diagnosis and prognosis of a living subject, but may also be used for postmortem diagnosis in a subject, for example, to identify family members at risk of developing the same disease.

A relevant cancer as used herein refers to B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and/or retinoblastoma.

### Proteins of the Invention

In one aspect of the invention, one-dimensional electrophoresis or isobaric tags for relative and absolute quantification (iTRAQ) or other appropriate methods are used to analyse relevant cancer tissue samples from a subject, such as a living subject, in order to measure the expression of the proteins of the invention for screening or diagnosis of a relevant cancer, to determine the prognosis of a patient with same, to monitor the effectiveness of therapy for same, or for drug development in relation to same.

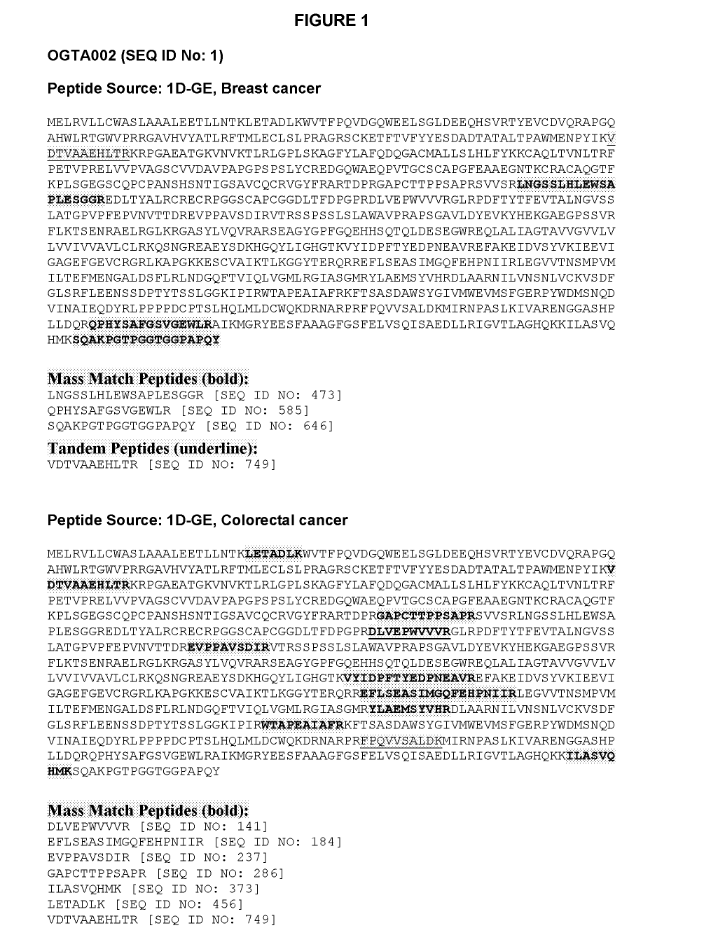

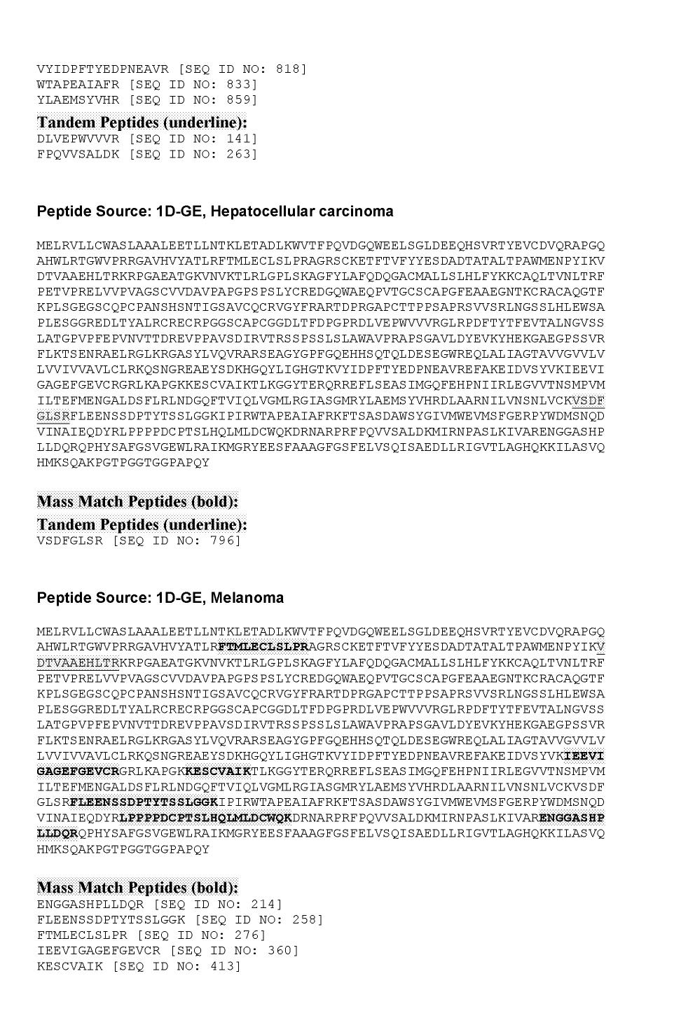

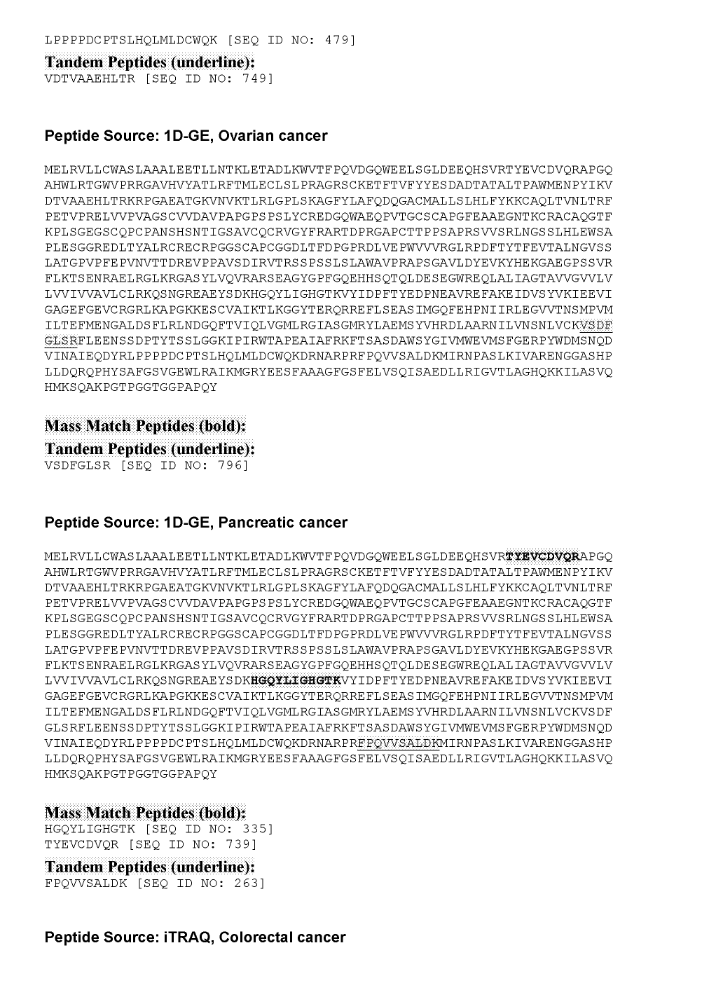

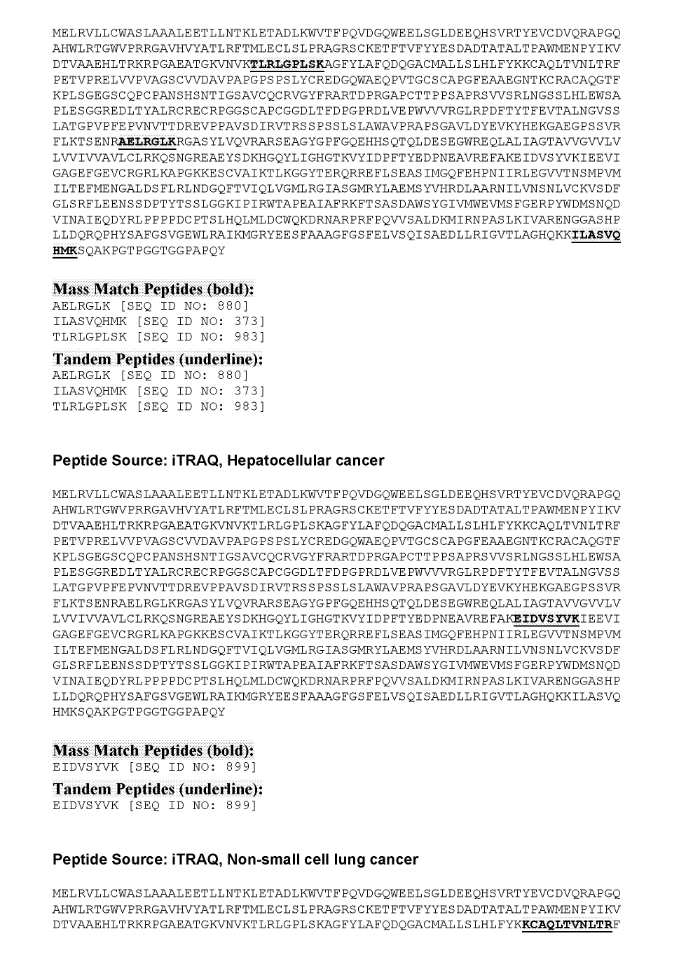

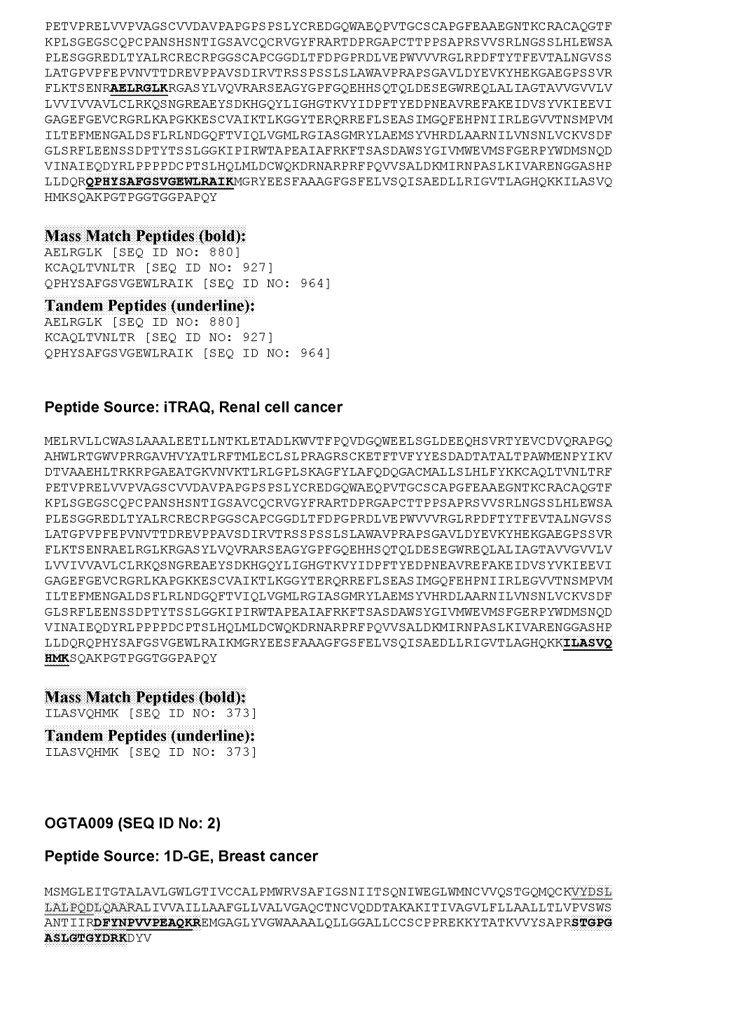

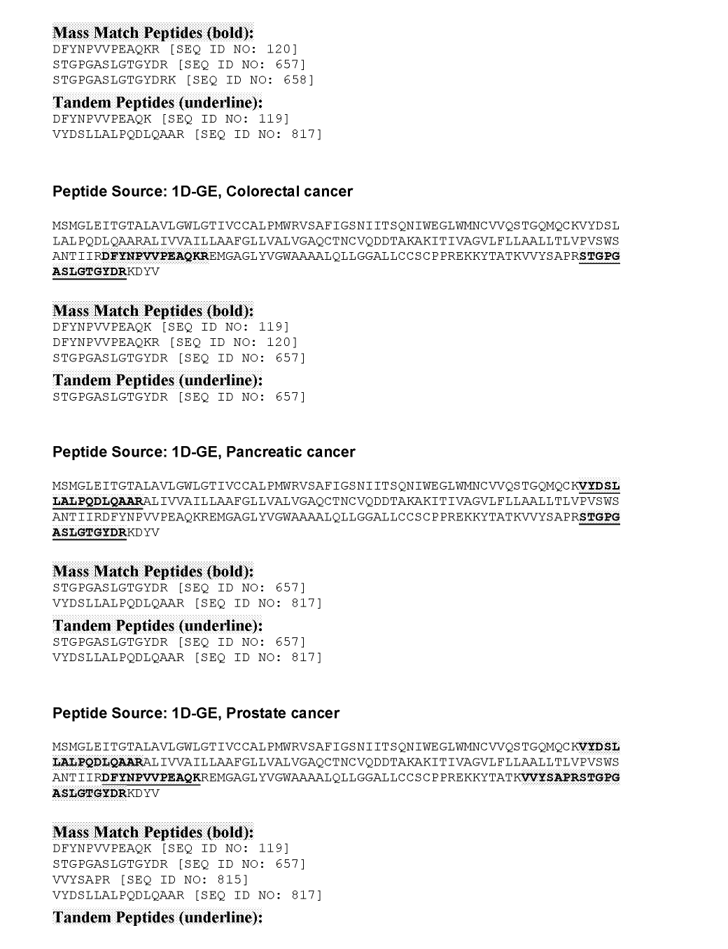

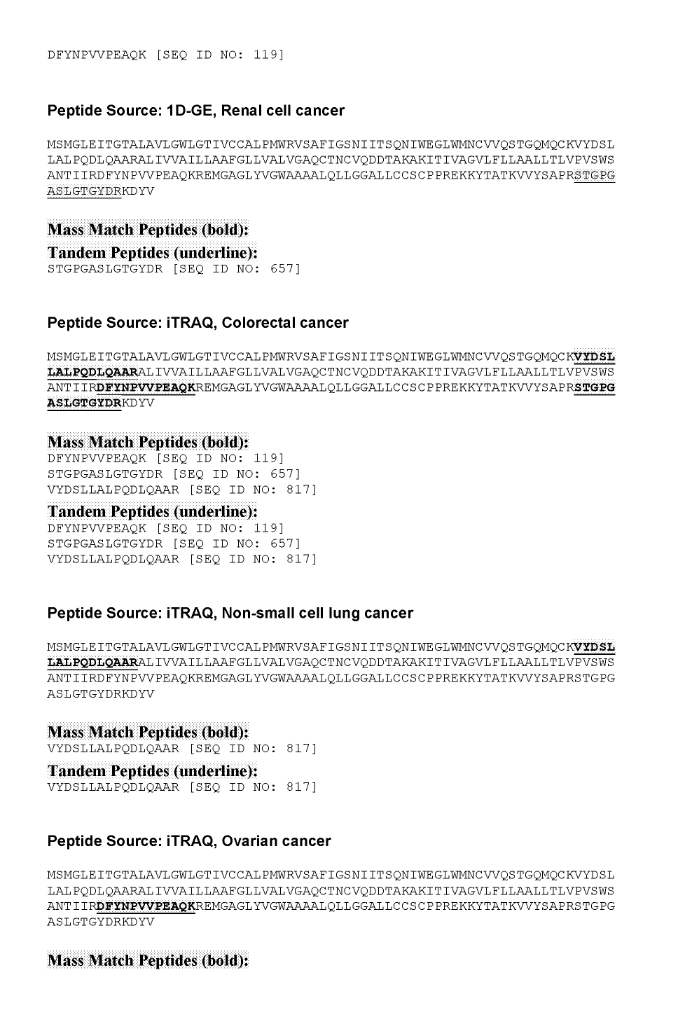

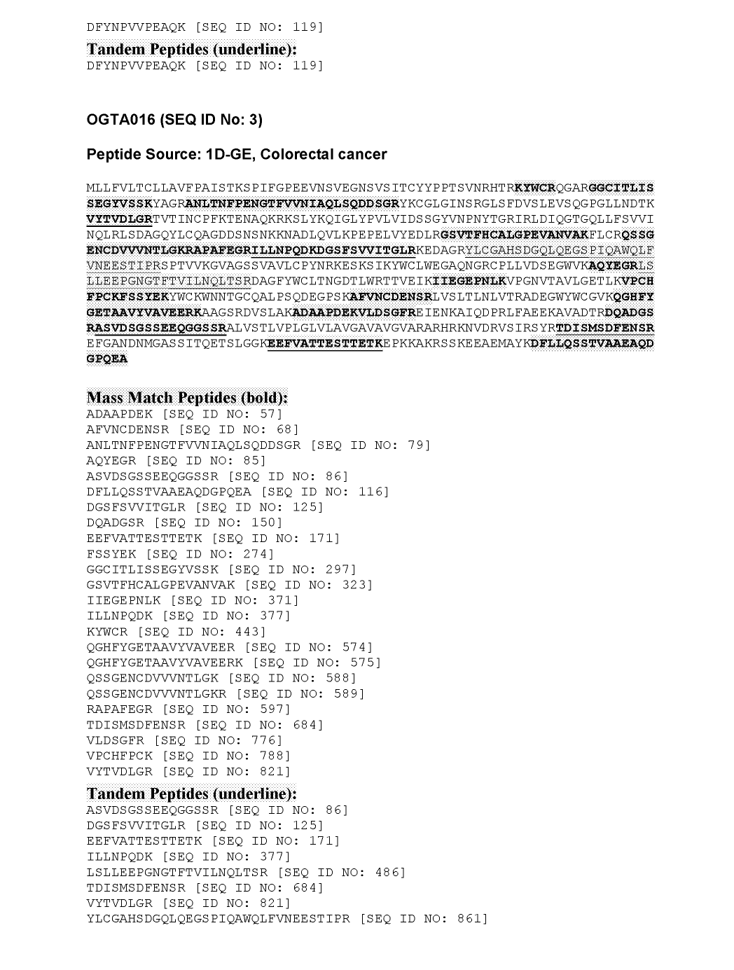

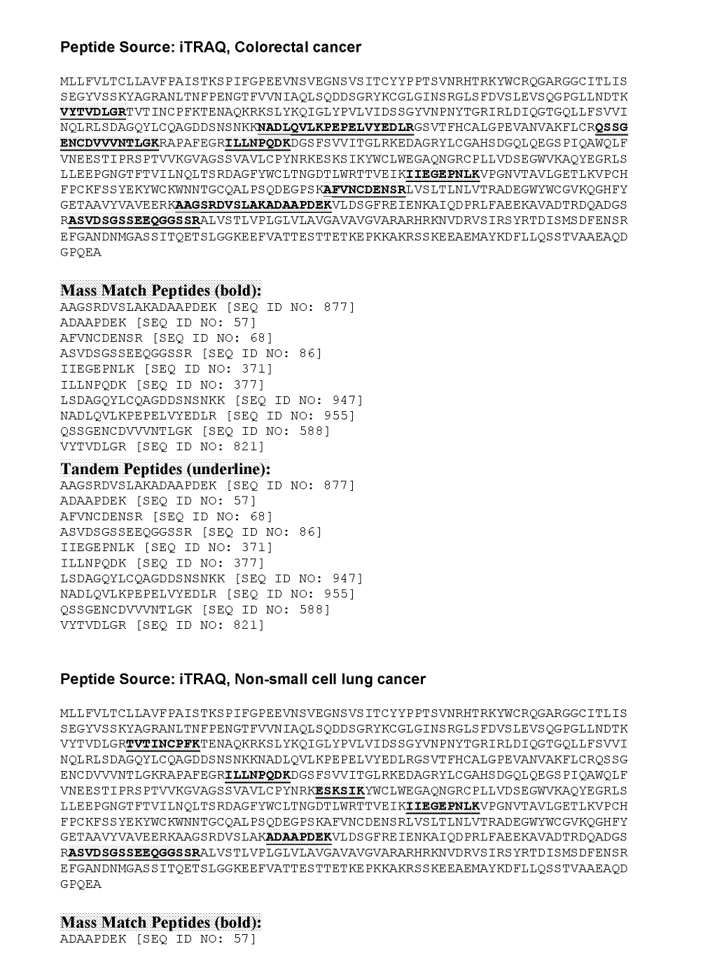

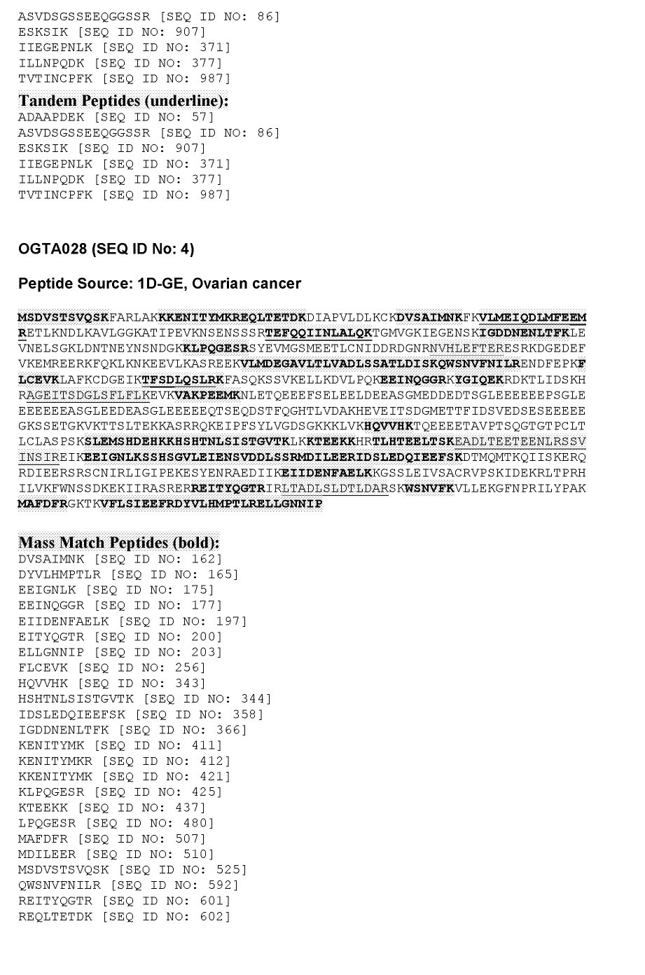

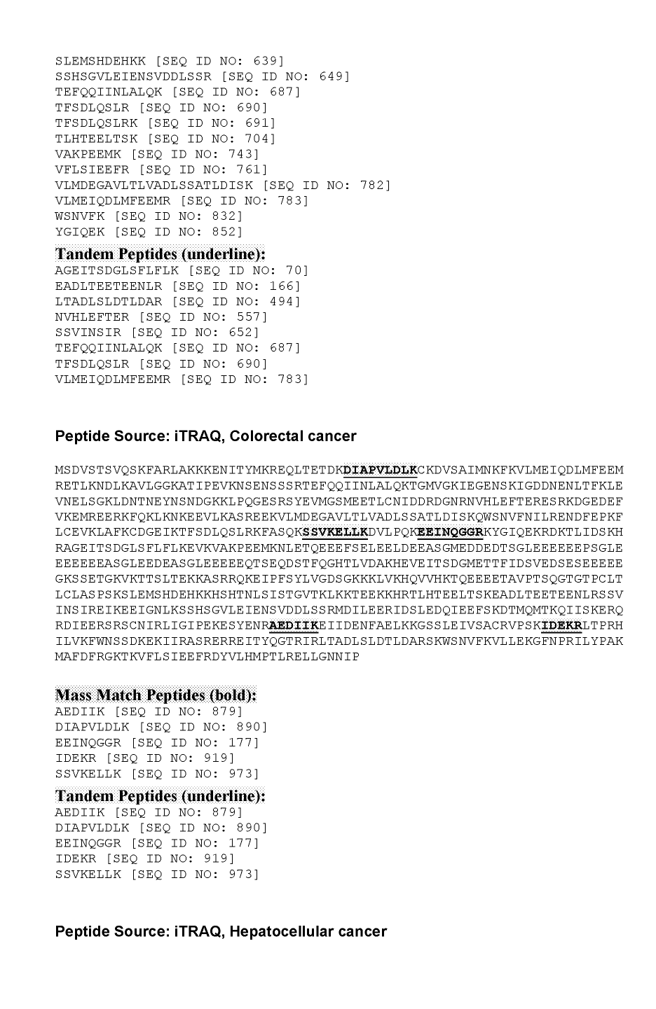

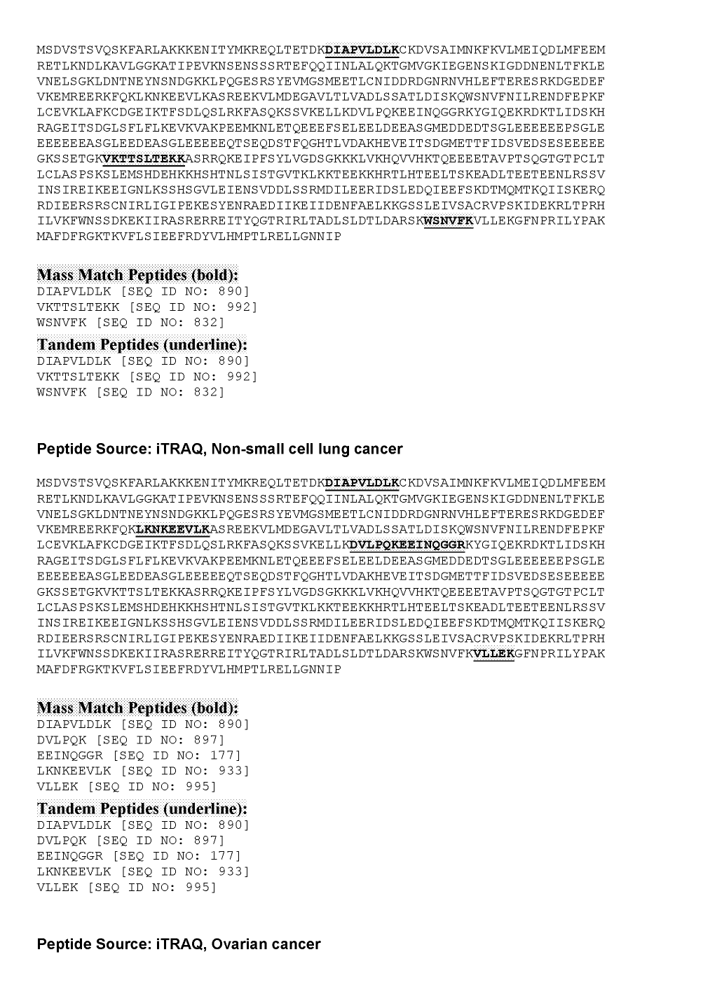

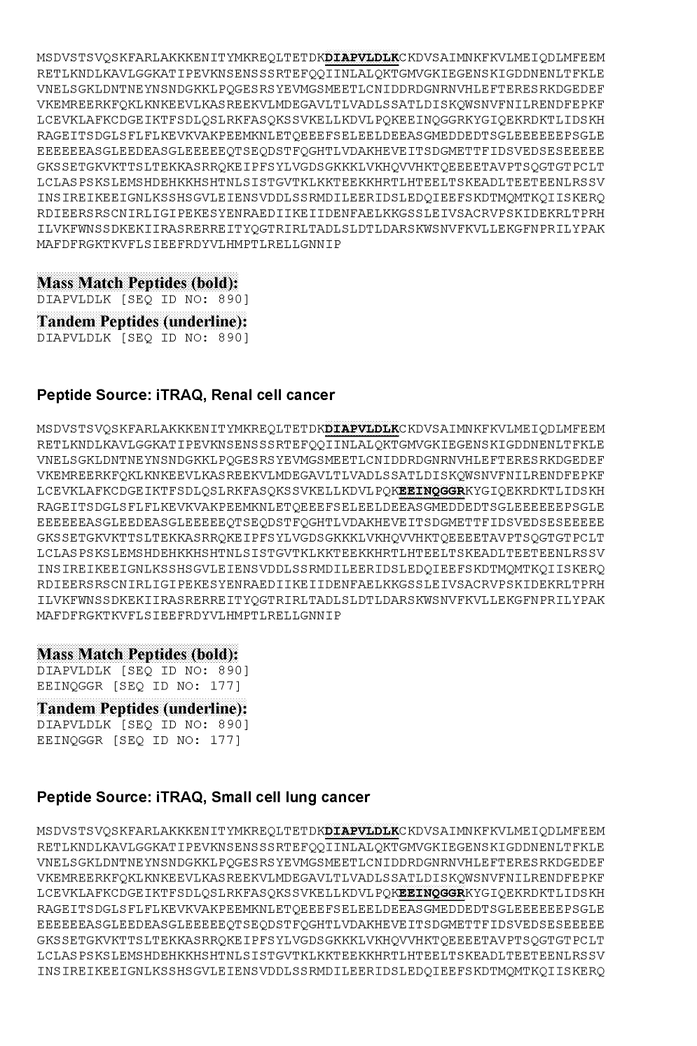

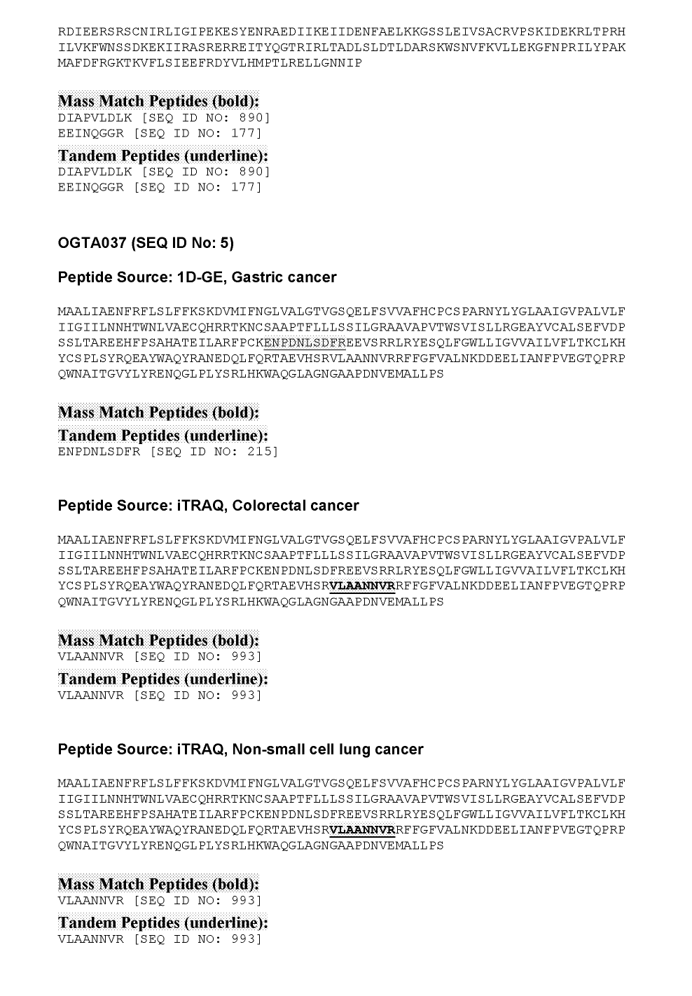

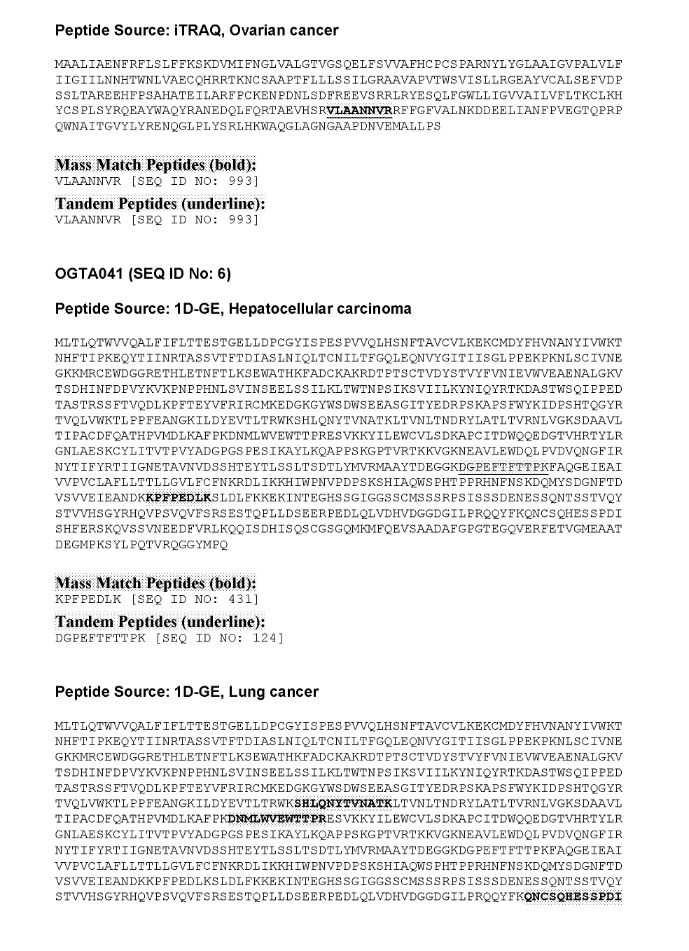

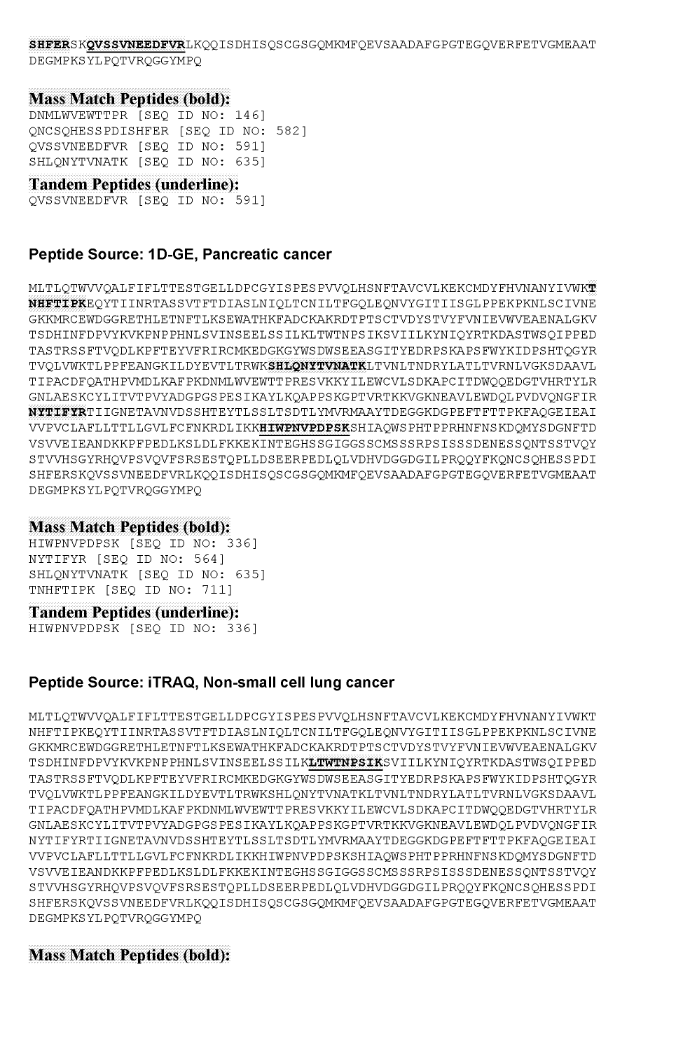

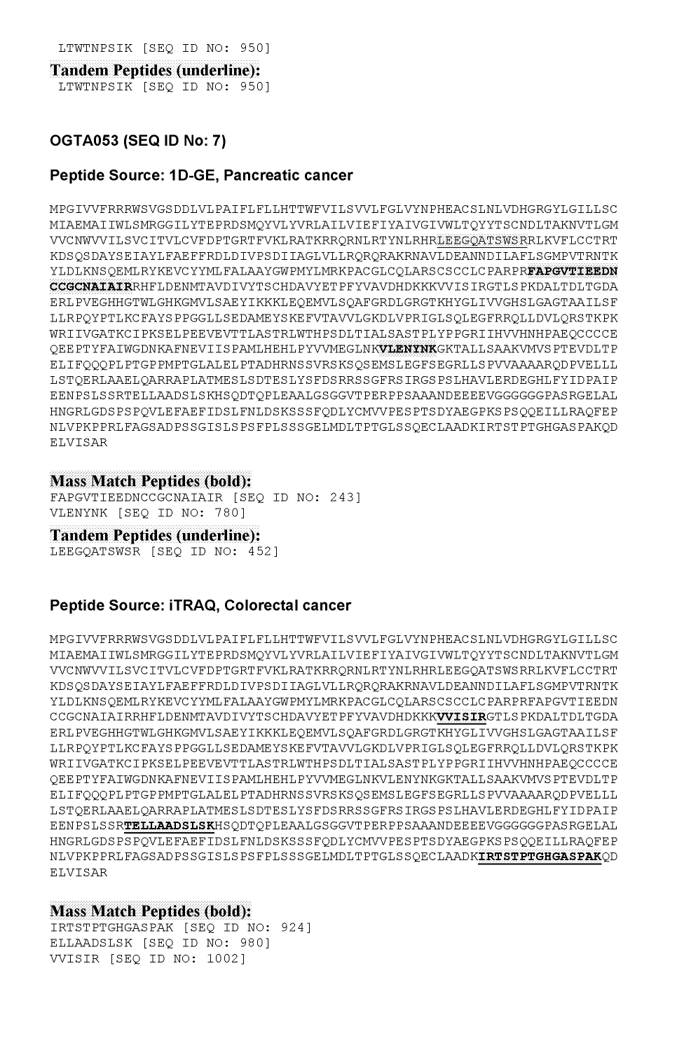

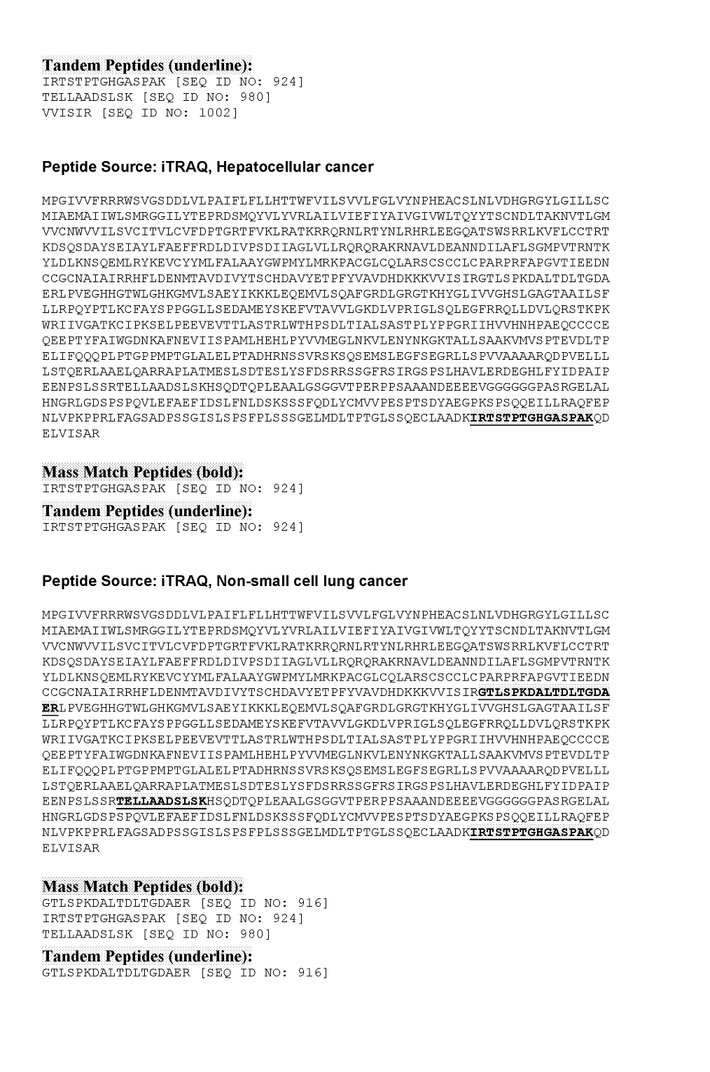

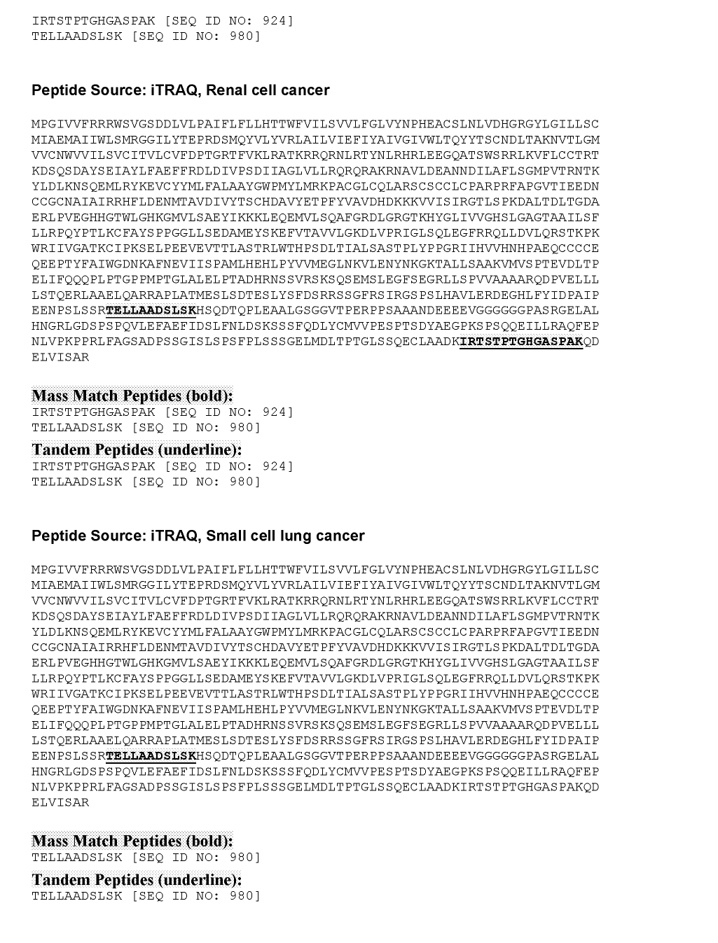

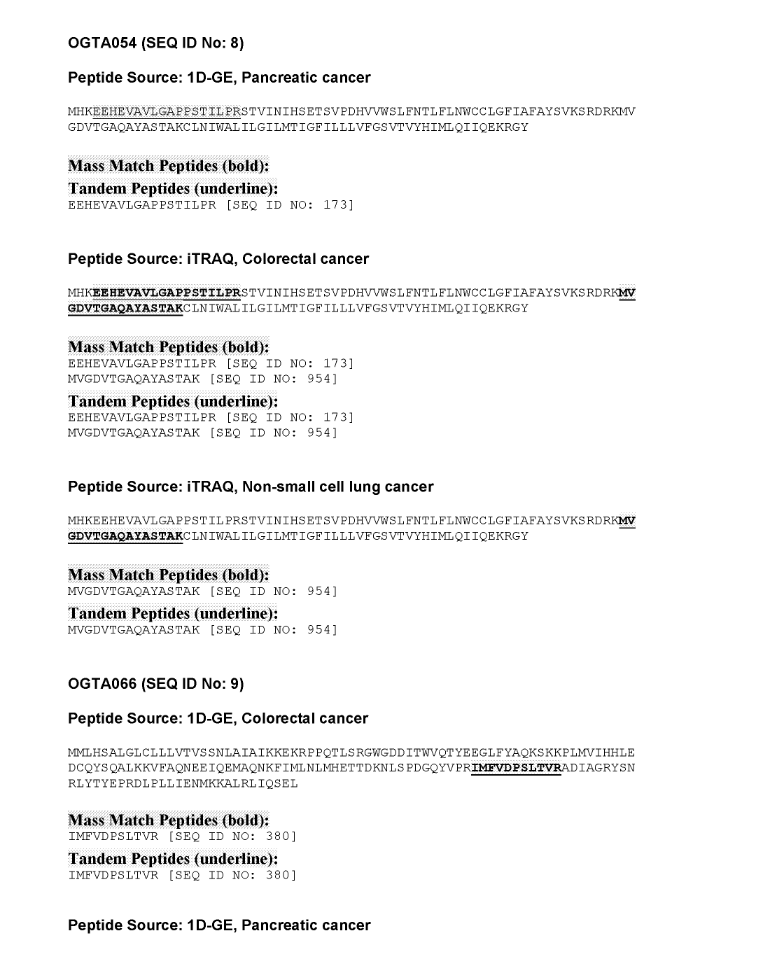

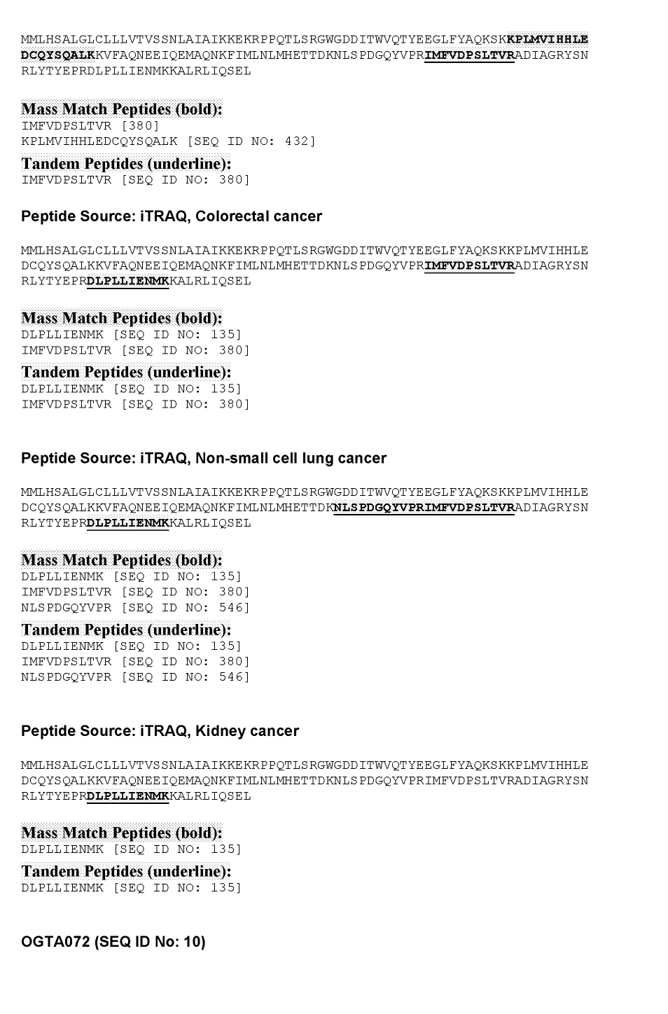

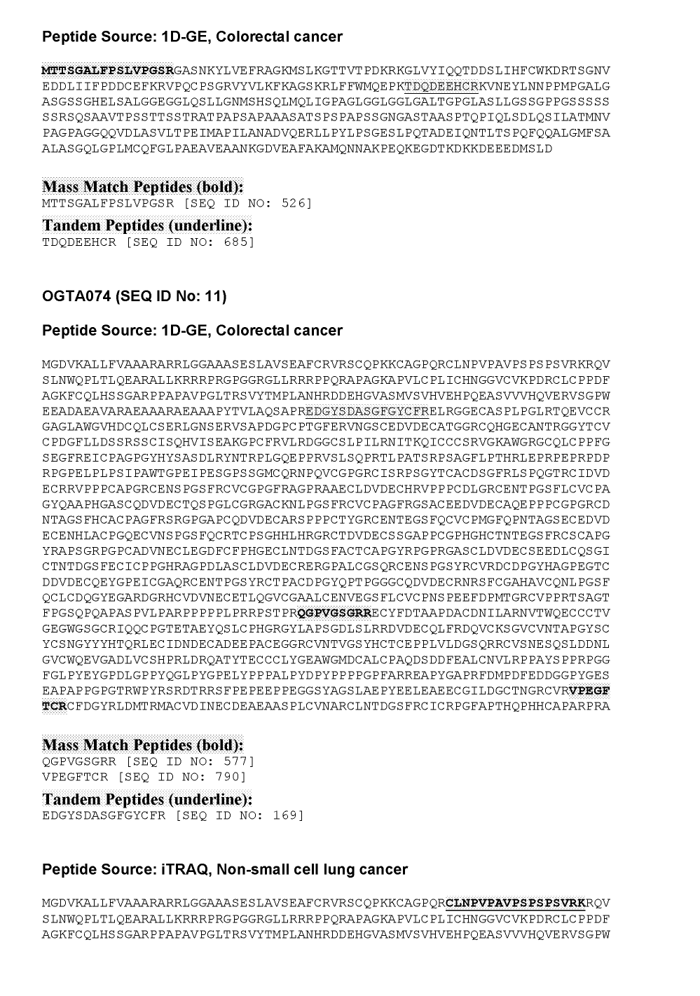

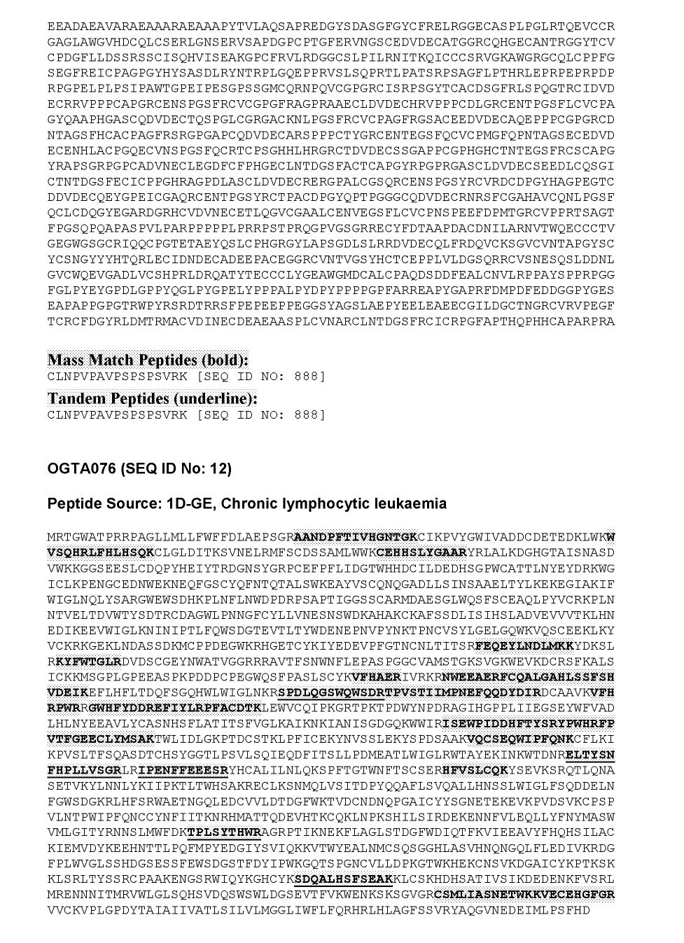

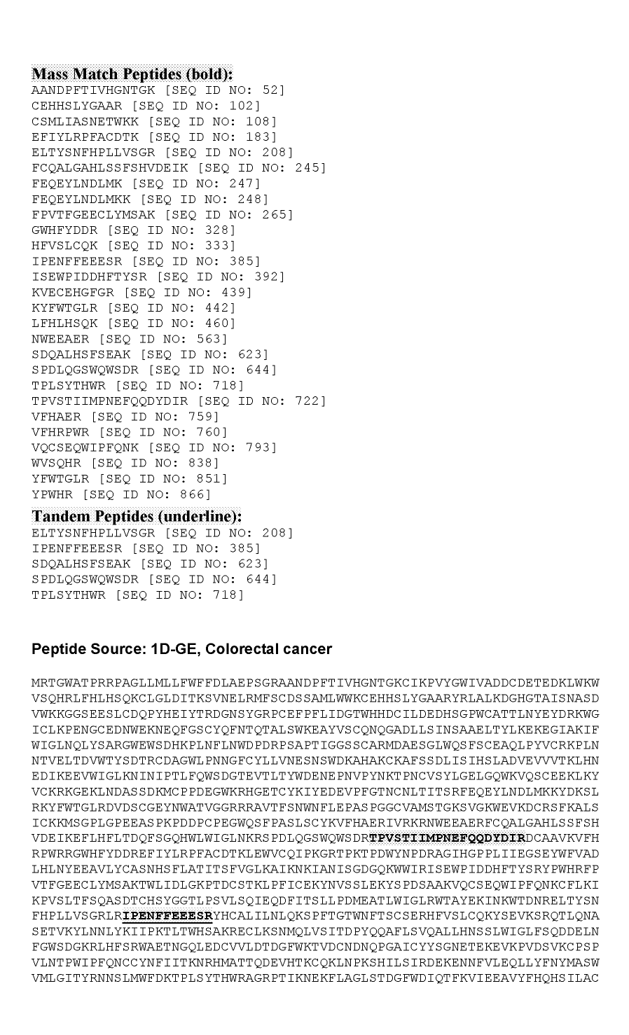

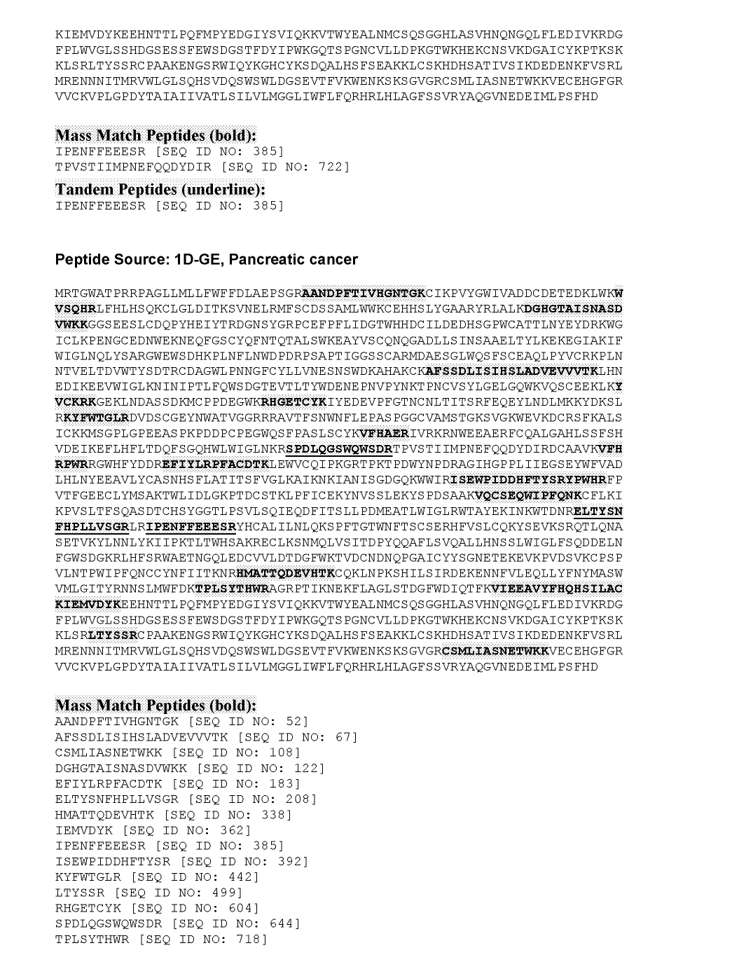

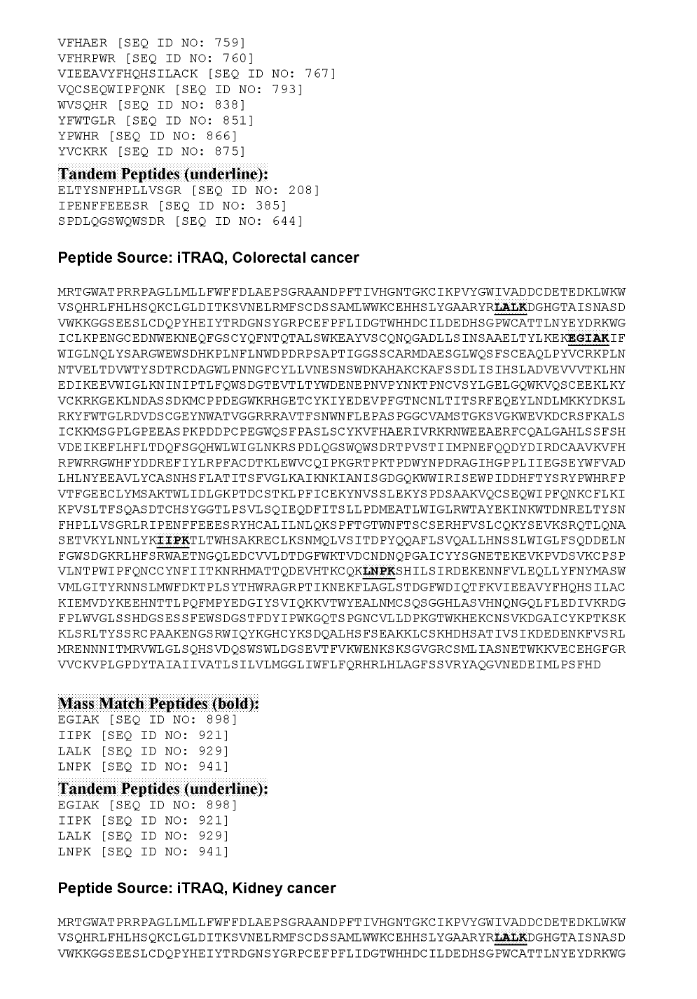

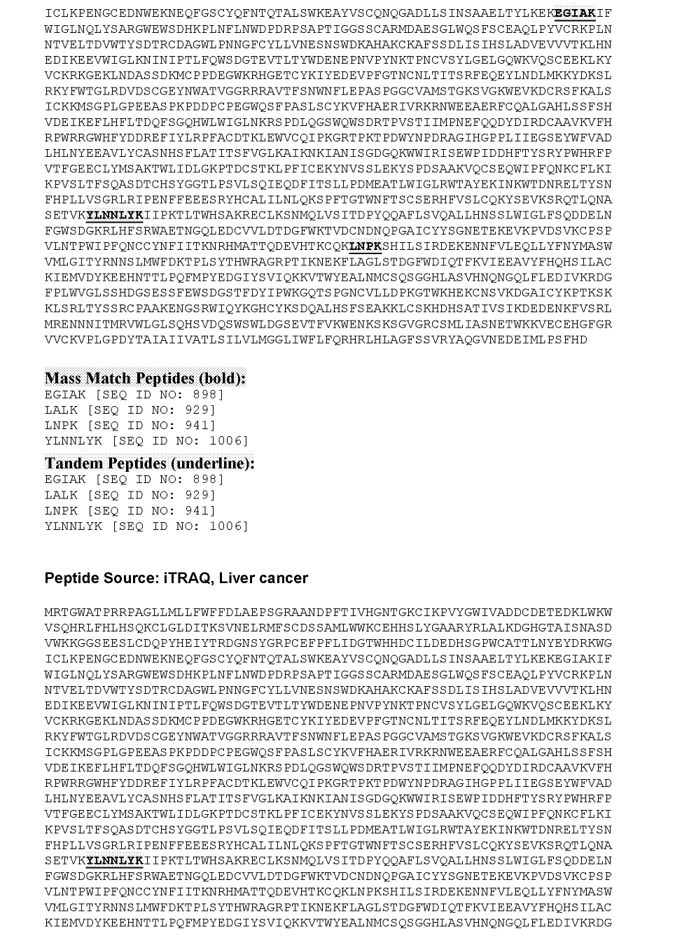

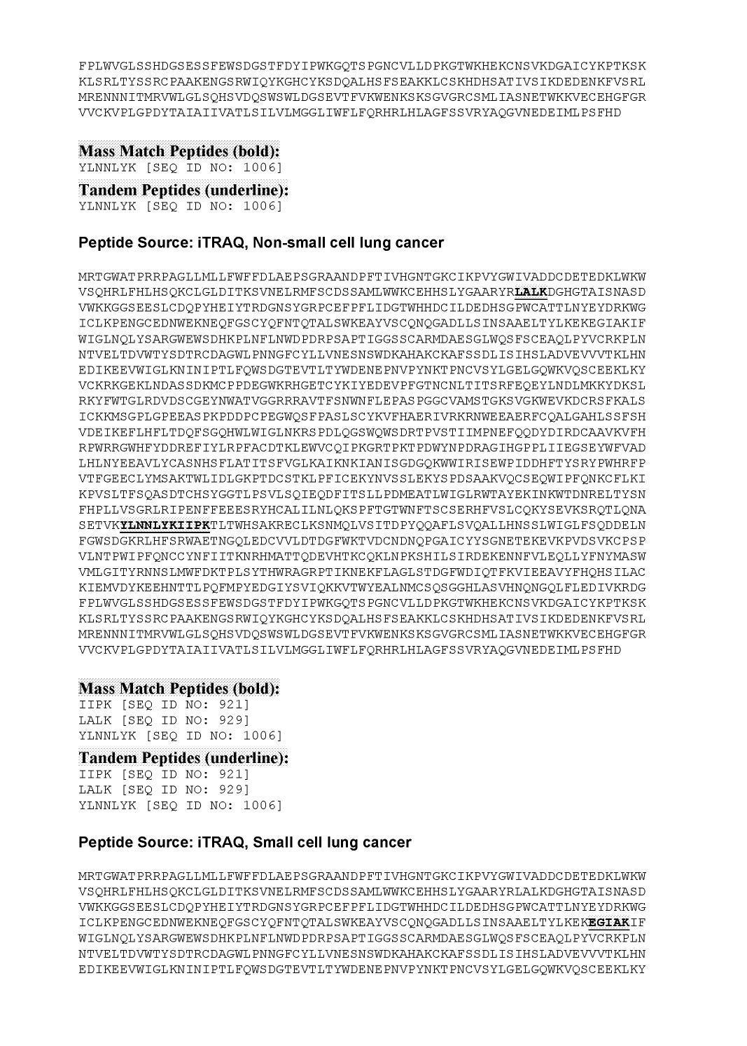

As used herein, the terms:
OGTA(s), or
OGTA according to the invention, or
OGTA employed in the invention or
“Proteins of the invention”,
   relates to “OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 and/or OGTA271” as illustrated in FIG. 1 detected experimentally by 1D gel electrophoresis and iTRAQ analysis of lymphoid, breast, cervical, colorectal, gastric, brain, liver, lung, skin, neuronal, osteoblast, ovarian, pancreatic, prostate, kidney and/or eye tissue samples. These terms are used interchangeably in this specification.

OGTA002 has been identified in membrane protein extracts of breast, colorectal, liver, skin, pancreatic, lung and kidney tissue samples from breast cancer, colorectal cancer, heaptocellular carcinoma, melanoma, ovarian cancer, pancreatic cancer, lung cancer and kidney cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P54760, Ephrin type-B receptor 4, was identified.

Ephrin type-B receptor 4 is known to be abundantly expressed in placenta and in a range of primary tissues and malignant cell lines. It is expressed in fetal, but not adult, brain, and in primitive and myeloid, but not lymphoid, hematopoietic cells. It is a receptor for members of the ephrin-B family. It binds to ephrin-B2. It may have a role in events mediating differentiation and development.

OGTA009 has been identified in membrane protein extracts of breast, pancreatic, prostate, kidney, colorectal, lung and ovarian tissue samples from breast cancer, pancreatic cancer, prostate cancer, renal cell cancer, colorectal cancer, lung cancer and ovarian cancer patients, through the methods and apparatus of the Preferred Technologies described in Examples 1 and 2 (1D gel electrophoresis or iTRAQ together with tryptic digest of membrane protein extracts).

Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: O15551, Claudin-3, was identified.

Claudin-3 plays a major role in tight junction-specific obliteration of the intercellular space, through calcium-independent cell-adhesion activity.

OGTA016 has been identified in membrane protein extracts of colorectal and lung tissue samples from colorectal cancer and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P01833, Polymeric-immunoglobulin receptor, was identified.

Polymeric-immunoglobulin receptor binds polymeric IgA and IgM at the basolateral surface of epithelial cells. The complex is then transported across the cell to be secreted at the apical surface. During this process a cleavage occurs that separates the extracellular (known as the secretory component) from the transmembrane segment.

OGTA028 has been identified in membrane protein extracts of ovarian, colorectal, kidney, liver and lung samples from ovarian cancer, colorectal cancer, kidney cancer, liver cancer and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q5T7N2, FLJ10884, was identified.

OGTA037 has been identified in membrane protein extracts of gastric, colorectal, lung and ovarian tissue samples from gastric cancer, colorectal cancer, lung cancer and ovarian cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q9HA72, Protein FAM26B, was identified.

OGTA041 has been identified in membrane protein extracts of liver, lung and pancreatic tissue samples from hepatocellular carcinoma, lung cancer and pancreatic cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P40189, Interleukin-6 receptor subunit beta, was identified.

Interleukin-6 receptor subunit beta is expressed in all the tissues and cell lines examined. Expression is not restricted to IL6 responsive cells. It is a signal-transducing molecule. The receptor systems for IL6, LIF, OSM, CNTF, IL11, CTF1 and BSF3 can utilize gp130 for initiating signal transmission. It binds to IL6/IL6R (alpha chain) complex, resulting in the formation of high-affinity IL6 binding sites, and transduces the signal. It does not bind IL6. It may have a role in embryonic development. The type I OSM receptor is capable of transducing OSM-specific signaling events.

OGTA053 has been identified in membrane protein extracts of pancreatic, colorectal, kidney, liver and lung tissue samples from pancreatic cancer, colorectal cancer, kidney cancer, liver cancer and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q9Y4D2, Snl-specific diacylglycerol lipase alpha, was identified. Snl-specific diacylglycerol lipase alpha is known to be highly expressed in the brain and pancreas. It catalyses the hydrolysis of diacylglycerol (DAG) to 2-arachidonoyl-glycerol (2-AG), the most abundant endocannabinoid in tissues. It is required for axonal growth during development and for retrograde synaptic signaling at mature synapses.

OGTA054 has been identified in membrane protein extracts of pancreatic, colorectal and lung tissue samples from pancreatic cancer, colorectal cancer and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P13164, Interferon-induced transmembrane protein 1, was identified.

Interferon-induced transmembrane protein 1 is implicated in the control of cell growth. It is a component of a multimeric complex involved in the transduction of antiproliferative and homotypic adhesion signals.

OGTA066 has been identified in membrane protein extracts of colorectal, pancreatic, kidney and lung tissue samples from colorectal cancer, pancreatic cancer, kidney cancer and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q8TD06, Anterior gradient protein 3 homolog, was identified.

OGTA072 has been identified in membrane protein extracts of colorectal tissue samples from colorectal cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q16186, Adhesion-regulating molecule 1, was identified. Adhesion-regulating molecule 1 promotes cell adhesion.

OGTA074 has been identified in membrane protein extracts of colorectal and lung tissue samples from colorectal cancer and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: O00508, Latent TGF-beta binding protein-4, was identified.

OGTA076 has been identified in membrane protein extracts of lymphoid, colorectal, pancreatic, kidney, liver, lung and ovarian tissue samples from chronic lymphocytic leukaemia, colorectal cancer, pancreatic cancer, kidney cancer, liver cancer, lung cancer and ovarian cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: O60449, Lymphocyte antigen 75, was identified.

Lymphocyte antigen 75 is known to be predominantly expressed in the spleen, thymus, colon and peripheral blood lymphocytes. It is detected in myeloid and B lymphoid cell lines. It is also expressed in malignant Hodgkin's lymphoma cells called Hodgkin's and Reed-Sternberg (HRS) cells. It acts as an endocytic receptor to direct captured antigens from the extracellular space to a specialized antigen-processing compartment. It causes reduced proliferation of B-lymphocytes.

OGTA085 has been identified in membrane protein extracts of lung, skin, eye and colorectal tissue samples from lung cancer, melanoma, retinoblastoma and colorectal cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q14318, 38 kDa FK506-binding protein homologue, was identified.

38 kDa FK506-binding protein homologue is known to be widely expressed, with the highest levels seen in the brain. It has no PPIase/rotamase activity.

OGTA087 has been identified in membrane protein extracts of lymphoid, breast, colorectal, lung, stomach and ovarian tissue samples from B-cell non-Hodgkin's lymphoma, breast cancer, colorectal cancer, gastric cancer, lung cancer, lymphoid leukaemia and ovarian cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: 094935, KIAA0851 protein, was identified.

OGTA088 has been identified in membrane protein extracts of breast, gastric, brain, liver, lung, lymphoid, pancreatic, kidney, eye and colorectal tissue samples from breast cancer, gastric cancer, glioblastoma, hepatocellular carcinoma, lung cancer, lymphoid leukaemia, pancreatic cancer, renal cell cancer, retinoblastoma and colorectal cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: O15126, Secretory carrier-associated membrane protein 1, was identified.

Secretory carrier-associated membrane protein 1 is known to be widely expressed, with the highest levels seen in the brain. It functions in post-Golgi recycling pathways. It acts as a recycling carrier to the cell surface.

OGTA089 has been identified in membrane protein extracts of breast, lung, ovarian and kidney tissue samples from breast cancer, lung cancer, ovarian cancer and kidney cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q5T225, Polycystic kidney disease 1-like, was identified.

OGTA091 has been identified in membrane protein extracts of breast, cervical, lymphoid, stomach, liver, lung, skin, bone, ovarian, pancreatic, prostate, kidney, eye and colorectal tissue samples from breast cancer, cervical cancer, chronic lymphocytic leukaemia, gastric cancer, hepatocellular carcinoma, lung cancer, melanoma, neuroblastoma, osteosarcoma, ovarian cancer, pancreatic cancer, prostate cancer, renal cell cancer, retinoblastoma and colorectal cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: O75923, Dysferlin, was identified.

Dysferlin is known to be highly expressed in skeletal muscle. It is also found in heart, placenta and at lower levels in liver, lung, kidney and pancreas.

OGTA098 has been identified in membrane protein extracts of breast, cervical, liver, lung, bone, pancreatic, prostate, kidney, colorectal and ovarian tissue samples from breast cancer, cervical cancer, hepatocellular carcinoma, lung cancer, osteosarcoma, pancreatic cancer, prostate cancer, renal cell cancer, colorectal cancer and ovarian cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q14126, Desmoglein-2, was identified. Desmoglein-2 is found in all of the tissues tested and carcinomas. It is a component of intercellular desmosome junctions. It is involved in the interaction of plaque proteins and intermediate filaments mediating cell-cell adhesion.

OGTA101 has been identified in membrane protein extracts of breast, cervical, colorectal, liver, lung, bone, pancreatic, kidney and ovarian tissue samples from breast cancer, cervical cancer, colorectal cancer, gastric cancer, hepatocellular carcinoma, lung cancer, osteosarcoma, pancreatic cancer, kidney cancer and ovarian cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P23468, Receptor-type tyrosine-protein phosphatase delta, was identified.

OGTA104 has been identified in membrane protein extracts of breast, liver, ovarian, pancreatic, colorectal, kidney and lung tissue samples from breast cancer, hepatocellular carcinoma, ovarian cancer, pancreatic cancer, colorectal cancer, kidney cancer and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q13443, ADAM 9, was identified.

ADAM 9 is known to be widely expressed. It is expressed in chondrocytes, liver and heart. It is a probable zinc protease. It may mediate cell-cell or cell-matrix interactions. It displays alpha-secretase activity for APP.

OGTA106 has been identified in membrane protein extracts of cervical, skin, pancreatic and liver tissue samples from cervical cancer, melanoma, pancreatic cancer and liver cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q99466, Neurogenic locus notch homologue protein 4, was identified.

Neurogenic locus notch homologue protein 4 is known to be highly expressed in the heart, moderately expressed in the lung and placenta and at low levels in the liver, skeletal muscle, kidney, pancreas, spleen, lymph node, thymus, bone marrow and fetal liver. No expression was seen in adult brain or peripheral blood leukocytes. It functions as a receptor for membrane-bound ligands Jagged1, Jagged2 and Delta1 to regulate cell-fate determination. Upon ligand activation through the released notch intracellular domain (NICD) it forms a transcriptional activator complex with RBP-J kappa and activates genes of the enhancer of split locus. It affects the implementation of differentiation, proliferation and apoptotic programs. It may regulate branching morphogenesis in the developing vascular system.

OGTA112 has been identified in membrane protein extracts of breast, cervical, lymphoid, liver, pancreatic and kidney tissue samples from breast cancer, cervical cancer, chronic lymphocytic leukaemia, hepatocellular carcinoma, pancreatic cancer and renal cell cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P48594, Serpin B4, was identified.

Serpin B4 is known to be predominantly expressed in squamous cells. It may act as a protease inhibitor to modulate the host immune response against tumour cells.

OGTA113 has been identified in membrane protein extracts of pancreatic, colorectal, kidney, lung and ovarian tissue samples from pancreatic cancer, colorectal cancer, kidney cancer, lung cancer and ovarian cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q9BTV4, Transmembrane protein 43, was identified.

OGTA119 has been identified in membrane protein extracts of lymphoid, breast, colorectal, stomach, liver, lung, brain, bone, pancreatic, prostate, kidney and ovarian tissue samples from B-cell non-Hodgkin's lymphoma, breast cancer, colorectal cancer, gastric cancer, hepatocellular carcinoma, lung cancer, lymphoid leukaemia, neuroblastoma, osteosarcoma, pancreatic cancer, prostate cancer, renal cell cancer and ovarian cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q6UKI4, KIAA1228 protein, was identified.

OGTA124 has been identified in membrane protein extracts of liver, lung, ovarian, pancreatic, kidney and colorectal tissue samples from hepatocellular carcinoma, lung cancer, ovarian cancer, pancreatic cancer, renal cell cancer and colorectal cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q9UP95, Solute carrier family 12 member 4, was identified.

Solute carrier family 12 member 4 is known to be ubiquitous. Levels are much higher in erythrocytes from patients with Hb SC and Hb SS compared to normal AA erythrocytes. This may contribute to red blood cell dehydration and to the manifestation of sickle cell disease by increasing the intracellular concentration of HbS. It mediates electroneutral potassium-chloride cotransport when activated by cell swelling. It may contribute to cell volume homeostasis in single cells. It may be involved in the regulation of basolateral Cl(−) exit in NaCl absorbing epithelia.

OGTA126 has been identified in membrane protein extracts of breast, colorectal, liver, skin, pancreatic, prostate, kidney and lung tissue samples from breast cancer, colorectal cancer, hepatocellular carcinoma, melanoma, pancreatic cancer, prostate cancer, renal cell cancer and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q9H5V8, CUB domain-containing protein 1, was identified.

CUB domain-containing protein 1 is known to be highly expressed in mitotic cells with low expression during interphase. It is detected at highest levels in skeletal muscle and colon with lower levels in kidney, small intestine, placenta and lung. It is expressed in a number of human tumour cell lines as well as in colorectal cancer, breast carcinoma and lung cancer. It is also expressed in cells with phenotypes reminiscent of mesenchymal stem cells and neural stem cells. It may be involved in cell adhesion and cell matrix association. It may play a role in the regulation of anchorage versus migration or proliferation versus differentiation via its phosphorylation. It may be a novel marker for leukaemia diagnosis and for immature hematopoietic stem cell subsets. It belongs to the tetraspanin web involved in tumour progression and metastasis.

OGTA156 has been identified in membrane protein extracts of lymphoid, liver, colorectal, kidney, lung and ovarian tissue samples from acute T-cell leukaemia, B-cell non-Hodgkin's lymphoma, chronic lymphocytic leukaemia, hepatocellular carcinoma, colorectal cancer, kidney cancer, lung cancer and ovarian cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P13612, Integrin alpha-4, was identified. Integrins alpha-4/beta-1 (VLA-4) and alpha-4/beta-7 are receptors for fibronectin. They recognize one or more domains within the alternatively spliced CS-1 and CS-5 regions of fibronectin. They are also receptors for VCAM1. Integrin alpha-4/beta-1 recognizes the sequence Q-I-D-S in VCAM1. Integrin alpha-4/beta-7 is also a receptor for MADCAM1. It recognizes the sequence L-D-T in MADCAM1. On activated endothelial cells integrin VLA-4 triggers homotypic aggregation for most VLA-4-positive leukocyte cell lines. It may also participate in cytolytic T-cell interactions with target cells.

OGTA159 has been identified in membrane protein extracts of breast, lymphoid, colorectal, liver, skin, pancreatic, eye, kidney and lung tissue samples from breast cancer, chronic lymphocytic leukaemia, colorectal cancer, hepatocellular carcinoma, melanoma, pancreatic cancer, kidney cancer and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q96HY6, Uncharacterized protein C20orf116, was identified.

OGTA168 has been identified in membrane protein extracts of brain and skin tissue samples from glioblastoma and melanoma patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q8WZA4, Collectin placenta 1, was identified.

OGTA169 has been identified in membrane protein extracts of breast, lymphoid, colorectal, kidney and lung tissue samples from breast cancer, chronic lymphocytic leukaemia, kidney cancer and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P20702, Integrin alpha-X, was identified.

Integrin alpha-X is known to be predominantly expressed in monocytes and granulocytes. Integrin alpha-X/beta-2 is a receptor for fibrinogen. It recognizes the sequence G-P-R in fibrinogen. It mediates cell-cell interaction during inflammatory responses. It is especially important in monocyte adhesion and chemotaxis.

OGTA174 has been identified in membrane protein extracts of lymphoid and lung tissue samples from acute T-cell leukaemia, chronic lymphocytic leukaemia and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: PO6127, T-cell surface glycoprotein CD5, was identified.

T-cell surface glycoprotein CD5 may act as a receptor in regulating T-cell proliferation. CD5 interacts with CD72/LYB-2.

OGTA176 has been identified in membrane protein extracts of lymphoid, colorectal, kidney and lung tissue samples from B-cell non-Hodgkin's lymphoma, chronic lymphocytic leukaemia, colorectal cancer, kidney cancer and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P21854, B-cell differentiation antigen CD72 homolog, was identified.

B-cell differentiation antigen CD72 is known to be predominantly expressed in pre-B-cells and B-cells but not terminally differentiated plasma cells. It plays a role in B-cell proliferation and differentiation. It associates with CD5.

OGTA177 has been identified in membrane protein extracts of lymphoid, colorectal, kidney, lung and ovarian tissue samples from chronic lymphocytic leukaemia, colorectal cancer, kidney cancer, lung cancer and ovarian cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P49961, Ectonucleoside triphosphate diphosphohydrolase 1, was identified.

Ectonucleoside triphosphate diphosphohydrolase 1 is known to be expressed primarily on activated lymphoid cells. It is also expressed in endothelial tissues. It is present in both placenta and umbilical vein. In the nervous system, it could hydrolyze ATP and other nucleotides to regulate purinergic neurotransmission. It could also be implicated in the prevention of platelet aggregation. It hydrolyses ATP and ADP equally well.

OGTA197 has been identified in membrane protein extracts of colorectal, liver, lung, skin, bone, ovarian, pancreatic, prostate and kidney tissue samples from colorectal cancer, hepatocellular carcinoma, lung cancer, melanoma, neuroblastoma, osteosarcoma, ovarian cancer, pancreatic cancer, prostate cancer, renal cell cancer and retinoblastoma patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P29317, Ephrin type-A receptor 2, was identified.

Ephrin type-A receptor 2 is known to be expressed most highly in tissues that contain a high proportion of epithelial cells e.g. skin, intestine, lung, and ovary. It is a receptor for members of the ephrin-A family. It binds to ephrin-A1, -A3, -A4 and -A5.

OGTA202 has been identified in membrane protein extracts of cancer tissue samples from cancer patients, through the methods and apparatus of the Preferred Technologies described in Examples 1 and 2 (1D gel electrophoresis or iTRAQ together with tryptic digest of membrane protein extracts) and through the methods and apparatus described in Example 5 (e.g. by liquid chromatography-mass spectrometry of membrane protein extracts). Peptide sequences were compared to the SWISS PROT and TrEMBL databases (held by the Swiss Institute of Bioinformatics (SIB) and the European Bioinformatics Institute (EBI) which are available at worldwide web expasy.org), and the entry P22223, Cadherin-3—CDH3, was identified. The nucleotide sequence encoding this protein is found at accession number NM_{—}001793, as given in SEQ ID NO: 1034.

Swiss Prot describes CDH3 as an 829 amino acid single pass type I membrane protein with 2 isoforms. The first isoform is the full length transcript which contains extra-cellular domain is located between residues 108-654 of SEQ ID NO: 35 and differs from the second isoform between residues 761-829 of SEQ ID NO: 35.

CDH3 is known to be expressed in several different cancers, including breast cancers of high histological grade (Hum Mol. Genet. 2010 Jul. 1; 19(13):2554-66), oesophageal and bladder cancer (Anticancer Res. 2009 October; 29(10):3945-7), endometrial cancer (J Clin Oncol. 2004 Apr. 1; 22(7):1242-52), colorectal and gastric carcinomas (Anticancer Res. 2009 October; 29(10):3945-7) and pancreatic cancer (Clin Cancer Res. 2008 Oct. 15; 14(20):6487-95). The inventor has shown CDH3 is expressed in breast and ovarian cancers and has also shown in vitro cell kill via a MabZAP assay in A431 cells (Example 7) suggesting affinity-based therapies directed against CDH3 in patients including those with breast and ovarian cancers will have a therapeutic effect. Immunohistochemistry experiments (Example 6) showed strong staining of breast cancer.

OGTA203 has been identified in membrane protein extracts of ovarian, kidney and lung tissue samples from ovarian cancer, renal cell cancer and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P55285, Cadherin-6, was identified.

Cadherin-6 is known to be highly expressed in brain, cerebellum, and kidney. Lung, pancreas, and gastric mucosa show a weak expression. It is also expressed in certain liver and kidney carcinomas. Cadherins are calcium dependent cell adhesion proteins. They preferentially interact with themselves in a homophilic manner in connecting cells; cadherins may thus contribute to the sorting of heterogeneous cell types.

OGTA206 has been identified in membrane protein extracts of breast, pancreatic, prostate, colorectal and lung tissue samples from breast cancer, pancreatic cancer, prostate cancer, colorectal cancer and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: O14493, Claudin-4, was identified.

Claudin-4 plays a major role in tight junction-specific obliteration of the intercellular space.

OGTA213 has been identified in membrane protein extracts of lung and colorectal tissue samples from lung cancer and colorectal cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q14767, Latent-transforming growth factor beta-binding protein 2, was identified.

Latent-transforming growth factor beta-binding protein 2 is known to be expressed in lung and weakly expressed in heart, placenta, liver and skeletal muscle. It may play an integral structural role in elastic-fiber architectural organization and/or assembly.

OGTA214 has been identified in membrane protein extracts of cancer tissue samples from cancer patients, through the methods and apparatus of the Preferred Technologies described in Examples 1 and 2 (1D gel electrophoresis or iTRAQ together with tryptic digest of membrane protein extracts) and through the methods and apparatus described in Example 8 (e.g. by liquid chromatography-mass spectrometry of membrane protein extracts). Peptide sequences were compared to the SWISS PROT and TrEMBL databases (held by the Swiss Institute of Bioinformatics (SIB) and the European Bioinformatics Institute (EBI) which are available at worldwide web expasy.org), and the entry Q9H3R2, Mucin-13—MUC13, was identified. The nucleotide sequence encoding this protein is found at accession number NM_{—}033049, as given in SEQ ID NO: 1033.

MUC13 is described by SWISS-PROT as a type I membrane protein which consists of one transmembrane region and an extracellular tail between amino acids 19-420 of SEQ ID No: 39. Previous studies have shown MUC13 mRNA is downregulated in epithelial cancers including the diseases of the invention (Int J Oncol. 2004 October; 25(4):1119-26). The inventor has shown MUC13 is expressed in cancer, suggesting affinity-based therapies directed against MUC13 in patients including those with cancer will have a therapeutic effect.

Immunohistochemical analysis (Example 9) revealed specific staining of tumor cells in gastric cancer (Prevalence ca. 75%) and colorectal cancer (Prevalence ca. 50%).

OGTA216 has been identified in membrane protein extracts of breast, pancreatic, colorectal, kidney, liver, lung and ovarian tissue samples from breast cancer, pancreatic cancer, colorectal cancer, kidney cancer, lung cancer and ovarian cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P55011, Solute carrier family 12 member 2, was identified.

Solute carrier family 12 member 2 is known to be expressed in many tissues. It is an electrically silent transporter system. It mediates sodium and chloride reabsorption. It plays a vital role in the regulation of ionic balance and cell volume.

OGTA222 has been identified in membrane protein extracts of lymphoid and colorectal tissue samples from B-cell non-Hodgkin's lymphoma and colorectal cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P16444, Dipeptidase 1, was identified.

Dipeptidase 1 hydrolyses a wide range of dipeptides. It is implicated in the renal metabolism of glutathione and its conjugates. It converts leukotriene D4 to leukotriene E4; it may play an important role in the regulation of leukotriene activity.

OGTA236 has been identified in membrane protein extracts of pancreatic and colorectal tissue samples from pancreatic cancer and colorectal cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P50443, Sulfate transporter, was identified.

Sulfate transporter is known to be ubiquitously expressed. It is a sulfate transporter. It may play a role in endochondral bone formation.

OGTA237 has been identified in membrane protein extracts of lymphoid, prostate, colorectal, kidney and lung tissue samples from B-cell non-Hodgkin's lymphoma, lymphoid leukaemia, prostate cancer, colorectal cancer, kidney cancer and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P22455, Fibroblast growth factor receptor 4, was identified.

Fibroblast growth factor receptor 4 is a receptor for acidic fibroblast growth factor. It does not bind to basic fibroblast growth factor. It binds FGF19.

OGTA247 has been identified in membrane protein extracts of liver, skin and lung tissue samples from hepatocellular carcinoma, melanoma and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P32004, Neural cell adhesion molecule L1, was identified.

Neural cell adhesion molecule L1 is a cell adhesion molecule with an important role in the development of the nervous system. It is involved in neuron-neuron adhesion, neurite fasciculation, outgrowth of neurites, etc. It binds to axonin on neurons.

OGTA248 has been identified in membrane protein extracts of kidney and lung tissue samples from renal cell cancer and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: P29376, Leukocyte tyrosine kinase receptor, was identified.

Leukocyte tyrosine kinase receptor is known to be expressed in non-hematopoietic cell lines and T- and B-cell lines. The exact function of this protein is not known. It is probably a receptor with a tyrosine-protein kinase activity.

OGTA249 has been identified in membrane protein extracts of ovarian, kidney and lung tissue samples from ovarian cancer, kidney cancer and lung cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q969L2, Protein MAL2, was identified.

Protein MAL2 is known to be predominantly expressed in kidney, lung, and liver. It is also found in thyroid gland, stomach and at lower levels in testis and small intestine. It is a member of the machinery of polarized transport. It is required for the indirect transcytotic route at the step of the egress of the transcytosing cargo from perinuclear endosomes in order for it to travel to the apical surface via a raft-dependent pathway.

OGTA257 has been identified in membrane protein extracts of pancreatic, colorectal, liver, lung and ovarian tissue samples from pancreatic cancer, colorectal cancer, liver cancer, lung cancer and ovarian cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q9H1D0, Transient receptor potential cation channel subfamily V member 6, was identified.

Transient receptor potential cation channel subfamily V member 6 is known to be expressed at high levels in the gastrointestinal tract, including esophagus, stomach, duodenum, jejunum, ileum and colon, and in pancreas, placenta, prostate and salivary gland. It is expressed at moderate levels in liver, kidney and testis. It is expressed in locally advanced prostate cancer, metastatic and androgen-insensitive prostatic lesions but not detected in healthy prostate tissue and benign prostatic hyperplasia. It is a calcium selective cation channel probably involved in Ca(2+) uptake in various tissues, including Ca(2+) reabsorption in intestine. The channel is activated by low internal calcium level, probably including intracellular calcium store depletion, and the current exhibits an inward rectification. Inactivation includes both a rapid Ca(2+)-dependent and a slower Ca(2+)-calmodulin-dependent mechanism, the latter may be regulated by phosphorylation. In vitro, is slowly inhibited by Mg(2+) in a voltage-independent manner. Heteromeric assembly with TRPV5 seems to modify channel properties. TRPV5-TRPV6 heteromultimeric concatemers exhibit voltage-dependent gating.

OGTA271 has been identified in membrane protein extracts of breast, cervical, colorectal, liver, lung, osteoblast, pancreatic and kidney tissue samples from breast cancer, cervical cancer, colorectal cancer, hepatocellular carcinoma, lung cancer, osteosarcoma, pancreatic cancer and renal cell cancer patients, through the methods and apparatus of the Preferred Technologies described in Examples 1 and 2 (1D gel electrophoresis or iTRAQ together with tryptic digest of membrane protein extracts). Peptide sequences were compared to the SWISS-PROT and trEMBL databases (held by the Swiss Institute of Bioinformatics (SIB) and the European Bioinformatics Institute (EBI) which are available at worldwide web expasy.com), and the following entry: Q5T021, Protein tyrosine phosphatase, receptor type, F, was identified.

Proteins of the invention are useful as are fragments e.g. antigenic or immunogenic fragments thereof and derivatives thereof. Epitope containing fragments including antigenic or immunogenic fragments will typically be of length 12 amino acids or more e.g. 20 amino acids or more e.g. 50 or 100 amino acids or more. Fragments may be 95% or more of the length of the full protein e.g. 90% or more e.g. 75% or 50% or 25% or 10% or more of the length of the full protein.

Eptiope containing fragments including antigenic or immunogenic fragments will be capable of eliciting a relevant immune response in a patient. DNA encoding proteins of the invention is also useful as are fragments thereof e.g. DNA encoding fragments of proteins of the invention such as immunogenic fragments thereof. Fragments of nucleic acid (e.g. DNA) encoding Proteins of the invention may be 95% or more of the length of the full coding region e.g. 90% or more e.g. 75% or 50% or 25% or 10% or more of the length of the full coding region. Fragments of nucleic acid (e.g. DNA) may be 36 nucleotides or more e.g. 60 nucleotides or more e.g. 150 or 300 nucleotides or more in length.

Derivatives of proteins of the invention include variants on the sequence in which one or more (e.g. 1-20 such as 15, or up to 20% such as up to 10% or 5% or 1% by number based on the total length of the protein) deletions, insertions or substitutions have been made.

Substitutions may typically be conservative substitutions. Derivatives of proteins of the invention include variants on the sequence which have a sequence identity over the entire length thereof of typically at least 60%, 70%, 80%, 90 or 95%. Derivatives will typically have essentially the same biological function as the protein from which they are derived. Derivatives will typically be comparably antigenic or immunogenic to the protein from which they are derived. Derivatives will typically have either the ligand-binding activity, or the active receptor-complex forming ability, or preferably both, of the protein from which they are derived.

Tables 1-48 below illustrate the different occurrences of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 and OGTA271 as detected by 1D gel electrophoresis and mass spectrometry of membrane protein extracts of lymphoid, breast, cervical, colorectal, gastric, brain, liver, lung, skin, neuronal, osteoblast, ovarian, pancreatic, prostate, kidney and eye tissue samples from relevant cancer patients. The first column provides the molecular weight, the second column gives information on the subfractionation protocol used, if any (see Example 1 below), and the last column provides a list of the sequences observed by mass spectrometry and the corresponding SEQ ID Nos.

Tables 49-93 illustrate the different occurrences of OGTA002, OGTA009, OGTA016, OGTA028, OGTA037, OGTA041, OGTA053, OGTA054, OGTA066, OGTA074, OGTA076, OGTA085, OGTA088, OGTA089, OGTA091, OGTA098, OGTA101, OGTA104, OGTA106, OGTA112, OGTA113, OGTA119, OGTA124, OGTA126, OGTA156, OGTA159, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 and OGTA271 as detected by iTRAQ and mass spectrometry of membrane protein extracts of colorectal, kidney, liver, lung and ovarian tissue samples from colorectal cancer, kidney cancer, liver cancer, lung cancer and ovarian cancer patients respectively. The first column provides the samples batch number, the second column gives the iTRAQ experiment number and the last column provides a list of the sequences observed by mass spectrometry and the corresponding SEQ ID Nos.

OGTA002

TABLE 1

| | | |
|---|---|---|
| MW | | |
| (Da) | Subfractionation | Tryptics identified [SEQ ID No] |

a-Breast cancer

| | |
|---|---|
| 88475 | LNGSSLHLEWSAPLESGGR [473], QPHYSAFGSVGEWLR [585], |
| | SQAKPGTPGGTGGPAPQY [646], VDTVAAEHLTR [749] |

b-Colorectal cancer

| | | |
|---|---|---|
| | Heparin Binding | DLVEPWVVVR [141], EFLSEASIMGQFEHPNIIR [184], |
| | | EVPPAVSDIR [237], LETADLK [456], VDTVAAEHLTR [749] |
| 46639 | Nucleotide Binding | FPQVVSALDK [263], ILASVQHMK [373], |
| | | VYIDPFTYEDPNEAVR [818], WTAPEAIAFR [833], |
| | | YLAEMSYVHR [859] |
| 119837 | Nucleotide Binding | FPQVVSALDK [263], GAPCTTPPSAPR [286] |

c-Hepatocellular carcinoma

| | |
|---|---|
| 88432 | VSDFGLSR [796] |

d-Melanoma

| | |
|---|---|
| 81352 | ENGGASHPLLDQR [214], FLEENSSDPTYTSSLGGK [258], |
| | FTMLECLSLPR [276], IEEVIGAGEFGEVCR [360], KESCVAIK |
| | [413], LPPPPDCPTSLHQLMLDCWQK [479], VDTVAAEHLTR |
| | [749] |

e-Ovarian cancer

| | |
|---|---|
| 69811 | VSDFGLSR [796] |

f-Pancreatic cancer

| | |
|---|---|
| 116302 | FPQVVSALDK [263], HGQYLIGHGTK [335], TYEVCDVQR |
| | [739] |

OGTA009

TABLE 2

| | | |
|---|---|---|
| MW | | |
| (Da) | Subfractionation | Tryptics identified [SEQ ID No] |

a-Breast cancer

| | | |
|---|---|---|
| 23489 | Vesicles | DFYNPVVPEAQK [119], DFYNPVVPEAQKR [120], |
| | | STGPGASLGTGYDR [657] |
| 23739 | Vesicles | DFYNPVVPEAQK [119] |
| 26156 | Vesicles | DFYNPVVPEAQK [119], STGPGASLGTGYDRK [658] |
| 27751 | Vesicles | VYDSLLALPQDLQAAR [817] |

b-Colorectal cancer

| | |
|---|---|
| 20920 | DFYNPVVPEAQKR [120], STGPGASLGTGYDR [657] |
| 21129 | DFYNPVVPEAQK [119], STGPGASLGTGYDR [657] |
| 21235 | STGPGASLGTGYDR [657] |
| 21342 | STGPGASLGTGYDR [657] |
| 21450 | STGPGASLGTGYDR [657] |
| 21560 | STGPGASLGTGYDR [657] |
| 21671 | STGPGASLGTGYDR [657] |

c-Pancreatic cancer

| | |
|---|---|
| 26738 | VYDSLLALPQDLQAAR [817] |
| 27019 | VYDSLLALPQDLQAAR [817] |
| 27306 | STGPGASLGTGYDR [657] |

d-Prostate cancer

| | |
|---|---|
| Digitonin | DFYNPVVPEAQK [120], STGPGASLGTGYDR [657], VVYSAPR |
| | [815], VYDSLLALPQDLQAAR [817] |

e-Renal cell cancer

| | |
|---|---|
| 19493 | STGPGASLGTGYDR [657] |

OGTA016

TABLE 3
Colorectal cancer

| MW | | |
|---|---|---|
| (Da) | Subfractionation | Tryptics identified [SEQ ID No] |
| | Heparin Binding | ADAAPDEK [57], DGSFSVVITGLR [125] |
| | Heparin Binding | AFVNCDENSR [68], ASVDSGSSEEQGGSSR [86], |
| | | DGSFSVVITGLR [125], ILLNPQDK [377], |
| | | QGHFYGETAAVYVAVEER [574] |
| | Heparin Binding | ANLTNFPENGTFVVNIAQLSQDDSGR [79], AQYEGR [85], |
| | | DGSFSVVITGLR [125], EEFVATTESTTETK [171], |
| | | QGHFYGETAAVYVAVEER [574], VYTVDLGR [821] |
| | Heparin Binding | DGSFSVVITGLR [125], GSVTFHCALGPEVANVAK [323], |
| | | ILLNPQDK [377], QGHFYGETAAVYVAVEER [574], |
| | | VYTVDLGR [821] |
| | Heparin Binding | DGSFSVVITGLR [125], QGHFYGETAAVYVAVEER [574] |
| | Heparin Binding | DGSFSVVITGLR [125], DQADGSR [150], IIEGEPNLK [371], |
| | | ILLNPQDK [377], QGHFYGETAAVYVAVEER [574], |
| | | TDISMSDFENSR [684], VPCHFPCK [788], VYTVDLGR [821] |
| 70703 | Nucleotide Binding | AQYEGR [85], EEFVATTESTTETK [171] |
| 84772 | Nucleotide Binding | DGSFSVVITGLR [125], FSSYEK [274], KYWCR [443], |
| | | QGHFYGETAAVYVAVEER [574] |
| 88695 | Nucleotide Binding | DGSFSVVITGLR [125], FSSYEK [274], |
| | | QGHFYGETAAVYVAVEER [574], |
| | | YLCGAHSDGQLQEGSPIQAWQLFVNEESTIPR [861] |
| 91099 | | DFLLQSSTVAAEAQDGPQEA [116], DGSFSVVITGLR [125], |
| | | EEFVATTESTTETK [171], QGHFYGETAAVYVAVEER [574], |
| | | TDISMSDFENSR [684], VYTVDLGR [821] |
| 91554 | Nucleotide Binding | ILLNPQDK [377], QGHFYGETAAVYVAVEER [574], |
| | | RAPAFEGR [597] |
| 93409 | | EEFVATTESTTETK [171], QGHFYGETAAVYVAVEER [574], |
| | | QSSGENCDVVVNTLGK [588], QSSGENCDVVVNTLGKR [589], |
| | | VLDSGFR [776], TDISMSDFENSR [684] |
| 95846 | | EEFVATTESTTETK [171], QGHFYGETAAVYVAVEER [574], |
| | | QGHFYGETAAVYVAVEERK [575], QSSGENCDVVVNTLGK |
| | | [588] |
| 107634 | Nucleotide Binding | DGSFSVVITGLR [125], EEFVATTESTTETK [171], ILLNPQDK |
| | | [377], QGHFYGETAAVYVAVEER [574], VYTVDLGR [821] |
| 109842 | Nucleotide Binding | ASVDSGSSEEQGGSSR [86], DGSFSVVITGLR [125], |
| | | EEFVATTESTTETK [171], FSSYEK [274], ILLNPQDK [377], |
| | | LSLLEEPGNGTFTVILNQLTSR [486], |
| | | QGHFYGETAAVYVAVEER [574], TDISMSDFENSR [684], |
| | | VYTVDLGR [821] |
| 117144 | Nucleotide Binding | ASVDSGSSEEQGGSSR [86], DGSFSVVITGLR [125], |
| | | GGCITLISSEGYVSSK [297], QGHFYGETAAVYVAVEER [574], |
| | | TDISMSDFENSR [684] |
| 125678 | Nucleotide Binding | EEFVATTESTTETK [171] |

OGTA028

TABLE 4
Ovarian cancer

| MW | | |
|---|---|---|
| (Da) | Subfractionation | Tryptics identified [SEQ ID No] |
| 22364 | | HQVVHK [343], KENITYMKR [412], MSDVSTSVQSK [525], |
| | | TEFQQIINLALQK [687], TFSDLQSLRK [691], VAKPEEMK [743], |
| | | VLMEIQDLMFEEMR [783] |
| 22505 | | EADLTEETEENLR [166], IDSLEDQIEEFSK [358], KENITYMKR |
| | | [412], VAKPEEMK [743], VFLSIEEFR [761] |
| 24585 | | EITYQGTR [200], KKENITYMK [421], KLPQGESR [425], |
| | | MDILEER [510], TFSDLQSLR [690], VFLSIEEFR [761], |
| | | VLMEIQDLMFEEMR [783] |
| 26031 | | KENITYMKR [412], KKENITYMK [421], KLPQGESR [425], |
| | | QWSNVFNILR [592], REQLTETDK [602], VLMEIQDLMFEEMR |
| | | [783] |
| 26256 | | EEINQGGR [177], FLCEVK [256], KKENITYMK [421], LPQGESR |
| | | [480], QWSNVFNILR [592], VLMEIQDLMFEEMR [783] |
| 26487 | | DYVLHMPTLR [165], EEINQGGR [177], IGDDNENLTFK [366], |
| | | KENITYMKR [412], KKENITYMK [421], LPQGESR [480], |
| | | NVHLEFTER [557], QWSNVFNILR [592], TEFQQIINLALQK |
| | | [687], VAKPEEMK [743], VLMEIQDLMFEEMR [783], YGIQEK |
| | | [852] |
| 26724 | | EEINQGGR [177], IGDDNENLTFK [366], KENITYMKR [412], |
| | | KKENITYMK [421], LPQGESR [480], QWSNVFNILR [592], |
| | | TEFQQIINLALQK [687], TLHTEELTSK [704], |
| | | VLMEIQDLMFEEMR [783], YGIQEK [852] |
| 26966 | | EEIGNLK [175], EIIDENFAELK [197], ELLGNNIP [203], |
| | | KENITYMK [411], KKENITYMK [421], LPQGESR [480], |
| | | QWSNVFNILR [592], TLHTEELTSK [704], |
| | | VLMDEGAVLTLVADLSSATLDISK [782], VLMEIQDLMFEEMR |
| | | [783] |
| 27470 | | AGEITSDGLSFLFLK [70], DYVLHMPTLR [165], ELLGNNIP |
| | | [203], KKENITYMK [421], KLPQGESR [425], YGIQEK [852] |
| 28000 | | AGEITSDGLSFLFLK [70], KKENITYMK [421] |
| 47275 | | DVSAIMNK [162], DYVLHMPTLR [165], EIIDENFAELK [197], |
| | | EITYQGTR [200], HSHTNLSISTGVTK [344], KKENITYMK [421], |
| | | KLPQGESR [425], KTEEKK [437], LPQGESR [480], |
| | | LTADLSLDTLDAR [494], REITYQGTR [601], |
| | | SSHSGVLEIENSVDDLSSR [649], SSVINSIR [652], TFSDLQSLR |
| | | [690], VFLSIEEFR [761], VLMEIQDLMFEEMR [783] |
| 48293 | | DYVLHMPTLR [165], EIIDENFAELK [197], EITYQGTR [200], |
| | | HSHTNLSISTGVTK [344], KLPQGESR [425], KTEEKK [437], |
| | | LTADLSLDTLDAR [494], MAFDFR [507], SLEMSHDEHKK [639], |
| | | VFLSIEEFR [761], WSNVFK [832] |

OGTA037

TABLE 5
Gastric cancer

| MW | | Tryptics identified |
|---|---|---|
| (Da) | Subfractionation | [SEQ ID No] |
| 24774 | | ENPDNLSDFR [215] |

OGTA041

TABLE 6

| | | |
|---|---|---|
| MW | | Tryptics identified |
| (Da) | Subfractionation | [SEQ ID No] |

a-Hepatocellular carcinoma

| | |
|---|---|
| 90607 | DGPEFTFTTPK [124], |
| | KPFPEDLK [431] |

b-Lung cancer

| | |
|---|---|
| 122412 | DNMLWVEWTTPR [146], |
| | QNCSQHESSPDISHFER [582], |
| | QVSSVNEEDFVR [591], |
| | SHLQNYTVNATK [635] |

c-Pancreatic cancer

| | |
|---|---|
| 104240 | HIWPNVPDPSK [336], |
| | NYTIFYR [564], |
| | SHLQNYTVNATK [635], |
| | TNHFTIPK [711] |

OGTA053

TABLE 7
Pancreatic cancer

| MW | | Tryptics identified |
|---|---|---|
| (Da) | Subfractionation | [SEQ ID No] |
| 67953 | | FAPGVTIEEDNCCGCNAIAIR [243], |
| | | LEEGQATSWSR [452], |
| | | VLENYNK [780] |

OGTA054

TABLE 8
Pancreatic cancer

| MW | | Tryptics identified |
|---|---|---|
| (Da) | Subfractionation | [SEQ ID No] |
| 9213 | | EEHEVAVLGAPPSTILPR [173] |

OGTA066

TABLE 9

| | | |
|---|---|---|
| MW | | Tryptics identified |
| (Da) | Subfractionation | [SEQ ID No] |

a-Colorectal cancer

| | |
|---|---|
| 19046 | IMFVDPSLTVR [380] |
| 19131 | IMFVDPSLTVR [380] |
| 19217 | IMFVDPSLTVR [380] |
| 19304 | IMFVDPSLTVR [380] |

c-Pancreatic cancer

| | |
|---|---|
| 24387 | IMFVDPSLTVR [380] |
| 24611 | IMFVDPSLTVR [380], |
| | KPLMVIHHLEDCQYSQALK [432] |

In one or more aspects of the invention OGTA does not relate to, for example use/methods of diagnosis for colorectal cancer.

OGTA072

TABLE 10
Colorectal cancer

| MW | | Tryptics identified |
|---|---|---|
| (Da) | Subfractionation | [SEQ ID No] |
| | Heparin Binding | MTTSGALFPSLVPGSR [526], |
| | | TDQDEEHCR [685] |

OGTA074

TABLE 11
Colorectal cancer

| MW | | Tryptics identified |
|---|---|---|
| (Da) | Subfractionation | [SEQ ID No] |
| 36031 | Nucleotide Binding | EDGYSDASGFGYCFR [169] |
| | Heparin Binding | EDGYSDASGFGYCFR [169], |
| | | QGPVGSGRR [577], |
| | | VPEGFTCR [790] |

OGTA076

TABLE 12

| | | |
|---|---|---|
| MW | | |
| (Da) | Subfractionation | Tryptics identified [SEQ ID No] |

a-Chronic lymphocytic leukaemia

| | | |
|---|---|---|
| 198008 | Chloroform | AANDPFTIVHGNTGK [52], CEHHSLYGAAR [102], |
| | Extracted | CSMLIASNETWKK [108], EFIYLRPFACDTK [183], |
| | | ELTYSNFHPLLVSGR [208], FCQALGAHLSSFSHVDEIK [245], |
| | | FEQEYLNDLMK [247], GWHFYDDR [328], IPENFFEEESR [385], |
| | | ISEWPIDDHFTYSR [392], KYFWTGLR [442], LFHLHSQK [460], |
| | | SPDLQGSWQWSDR [644], TPLSYTHWR [718[, |
| | | TPVSTIIMPNEFQQDYDIR [722], VFHRPWR [760], |
| | | VQCSEQWIPFQNK [793], WVSQHR [838], YFWTGLR [851], |
| | | YPWHR [866] |
| 223568 | | AANDPFTIVHGNTGK [52], CSMLIASNETWKK [108], |
| | | ELTYSNFHPLLVSGR [208], FEQEYLNDLMKK [248], |
| | | FPVTFGEECLYMSAK [265], GWHFYDDR [328], HFVSLCQK [333], |
| | | IPENFFEEESR [385], ISEWPIDDHFTYSR [392], |
| | | KVECEHGFGR [439], NWEEAER [563], SDQALHSFSEAK [623], |
| | | SPDLQGSWQWSDR [644], TPLSYTHWR [718], VFHAER [759], |
| | | VQCSEQWIPFQNK [793], WVSQHR [838], YPWHR [866] |
| 238644 | | KVECEHGFGR [439], SDQALHSFSEAK +623+, |
| | | TPLSYTHWR [718], VFHAER ]759] |

b-Colorectal cancer

| | |
|---|---|
| 167535 | IPENFFEEESR [385], TPVSTIIMPNEFQQDYDIR [722] |

c-Pancreatic cancer

| | |
|---|---|
| 161314 | AANDPFTIVHGNTGK [52], EFIYLRPFACDTK [183], |
| | ELTYSNFHPLLVSGR [208], IEMVDYK [362], IPENFFEEESR [385], |
| | ISEWPIDDHFTYSR [392], LTYSSR [499], RHGETCYK [604], |
| | SPDLQGSWQWSDR [644], TPLSYTHWR [718], VFHAER [759], |
| | VFHRPWR [760], VQCSEQWIPFQNK [793], WVSQHR [838], |
| | YFWTGLR [851], YPWHR [866], YVCKRK [875] |
| 261976 | AFSSDLISIHSLADVEVVVTK [67], CSMLIASNETWKK [108], |
| | DGHGTAISNASDVWKK [122], EFIYLRPFACDTK [183], |
| | HMATTQDEVHTK [338], IPENFFEEESR [385], |
| | ISEWPIDDHFTYSR [392], KYFWTGLR [442], |
| | SPDLQGSWQWSDR [644], TPLSYTHWR [718], VFHRPWR [760], |
| | VIEEAVYFHQHSILACK [767], VQCSEQWIPFQNK [793], |
| | YFWTGLR [851], YPWHR [866], YVCKRK [875] |

OGTA085

TABLE 13

| | | |
|---|---|---|
| MW | | |
| (Da) | Subfractionation | Tryptics identified [SEQ ID No] |

a-Lung cancer

| | |
|---|---|
| 31005 | KMLGNPSR [427], STETALYR [655], TIHAELSK [700], |
| | VLAQQGEYSEAIPILR [773] |
| 64045 | TAVDGPDLEMLTGQER [677], VLAQQGEYSEAIPILR [773] |
| 69488 | TAVDGPDLEMLTGQER [677], VLAQQGEYSEAIPILR [773] |

b-Melanoma

| | |
|---|---|
| 22017 | KMLGNPSR [427], MGQPPAEEAEQPGALAR [516], |
| | STETALYR [655], TAVDGPDLEMLTGQER [677], |
| | VDMTFEEEAQLLQLK [748] |
| 22380 | STETALYRK [656], TAVDGPDLEMLTGQER [677], |
| | VDMTFEEEAQLLQLK [748] |
| 22505 | SCSLVLEHQPDNIK [620], STETALYRK [656], |
| | TAVDGPDLEMLTGQER [677] |
| 22632 | SCSLVLEHQPDNIK [620], TAVDGPDLEMLTGQER [677], |
| | VDMTFEEEAQLLQLK [748] |
| 27711 | ADFVLAANSYDLAIK [60], GQVVTVHLQTSLENGTR [313], |
| | KMLGNPSR [427], LDHYR [447], TAVDGPDLEMLTGQER [677] |

c-Retinoblastoma

| | |
|---|---|
| 50346 | KMLGNPSR [427], MGQPPAEEAEQPGALAR [516], |
| | VLAQQGEYSEAIPILR [773] |

OGTA087

TABLE 14

| | | |
|---|---|---|
| MW | | |
| (Da) | Subfractionation | Tryptics identified [SEQ ID No] |

a-B-cell non-Hodgkin's lymphoma

| | |
|---|---|
| 54163 | FVWNGHLLR [279], LGVLHVGQK [464], LHITPEK [466], |
| | LSNTSPEFQEMSLLER [488], NNFSDGFR [550], YIAFDFHK [854], |
| | YKPLPQISK [856] |
| 55443 | ATDFDVLSYKK [89], FVWNGHLLR [279], |
| | KTMLHLTDIQLQDNK [438], LHITPEK [466], NNFSDGFR [550], |
| | QVIINLINQK [590], QYAGTGALK [593], VANHMDGFQR [744], |
| | YKPLPQISK [856] |
| 75222 | LHITPEK [466], NNFSDGFR [550], QVIINLINQK [590], |
| | QYAGTGALK [593], VANHMDGFQR [744], YKPLPQISK [856] |

b-Breast cancer

| | |
|---|---|
| 52857 | ATDFDVLSYKK [89], FVWNGHLLR [279], TNVIQSLLAR [714] |

c-Colorectal cancer

| | |
|---|---|
| Heparin Binding | LHITPEK [466], YIAFDFHK [854] |

d-Gastric cancer

| | |
|---|---|
| 50390 | TNVIQSLLAR [714] |
| 50979 | TNVIQSLLAR [714] |

e-Lung cancer

| | |
|---|---|
| 34397 | LSNTSPEFQEMSLLER [488], NNFSDGFR [550], YFDWILISR [848], |
| | YFDWILISRR [849], YIAFDFHK [854], YKPLPQISK [856] |
| 63738 | HFDSQVIIYGK [332], LHITPEK [466], NNFSDGFR [550], |
| | RTHLGLIMDGWNSMIR [615], TNVIQSLLAR [714] |
| 66502 | GSEKPLEQTFATMVSSLGSGMMR [314], HFDSQVIIYGK [332], |
| | LHITPEK [466], NNFSDGFR [550], QYAGTGALK [593], |
| | VANHMDGFQR [744], YIAFDFHK [854] |
| 69051 | ATDFDVLSYK [88], GSEKPLEQTFATMVSSLGSGMMR [314], |
| | LEEQDEFEK [453], THLGLIMDGWNSMIR [696], |
| | VSTEVTLAVK [797] |

f-Lymphoid leukaemia, unspecified

| | |
|---|---|
| 56356 | TNVIQSLLAR [714], YKPLPQISK [856] |
| 58422 | LSNTSPEFQEMSLLER [488], NNFSDGFR [550], |
| | THLGLIMDGWNSMIR [696] |

g-Ovarian cancer

| | |
|---|---|
| 42632 | ATDFDVLSYKK [89], FVWNGHLLR [279], GSIPVFWSQRPNLK [316], |
| | HFDSQVIIYGK [332], KTMLHLTDIQLQDNK [438], |
| | LEEQDEFEK [453], RTHLGLIMDGWNSMIR [615], |
| | TMLHLTDIQLQDNK [707] |

OGTA088

TABLE 15

| | | |
|---|---|---|
| MW | | |
| (Da) | Subfractionation | Tryptics identified [SEQ ID No] |

a-Breast cancer

| | |
|---|---|
| 29702 | EHALAQAELLK [190], EMQNLSQHGR [211], |
| | NVPPGLDEYNPFSDSR [560], RQEELER [612] |
| 29901 | DPSVTQVTR [149], EMQNLSQHGR [211], |
| | NVPPGLDEYNPFSDSR [560], RQEELER [612] |
| 30103 | EHALAQAELLK [190], EMQNLSQHGR [211], |
| | NVPPGLDEYNPFSDSR [560], RQEELER [612] |
| 30309 | EMQNLSQHGR [211], NVPPGLDEYNPFSDSR [560], |
| | RQEELER [612] |

b-Gastric cancer

| | |
|---|---|
| 29509 | EHALAQAELLK [190], EMQNLSQHGR [211] |

c-Glioblastoma

| | |
|---|---|
| 29544 | EHALAQAELLK [190], EMQNLSQHGR [211], KAAELDR [405], |
| | MPNVPNTQPAIMKPTEEHPAYTQIAK [522], |
| | NVPPGLDEYNPFSDSR [560], QEELER [566], RQEELER [612], |
| | TPPPGGVK [721], TVQTAAANAASTAASSAAQNAFK [737], |
| | VHGLYR [766] |
| 29779 | EHALAQAELLK [190], EMQNLSQHGR [211], |
| | NVPPGLDEYNPFSDSR [560], RQEELER [612] |
| 30027 | AQQEFATGVMSNK [84], EHALAQAELLK [190], |
| | EMQNLSQHGR [211], NVPPGLDEYNPFSDSR [560], RQEELER [612], |
| | TVQTAAANAASTAASSAAQNAFK [737], VHGLYR [766] |

d-Hepatocellular carcinoma

| | |
|---|---|
| 26264 | DPSVTQVTR [149], EHALAQAELLK [190], EMQNLSQHGR [211], |
| | KAAELDRR [406], NVPPGLDEYNPFSDSR [560], RQEELER [612] |
| 26522 | EMQNLSQHGR [211], NVPPGLDEYNPFSDSR [560] |
| 27053 | EMQNLSQHGR [211], EMQNLSQHGRK [212], |
| | NVPPGLDEYNPFSDSR [560], RQEELERK [613] |
| 28538 | EHALAQAELLK [190], EMQNLSQHGR [211], |
| | MPNVPNTQPAIMKPTEEHPAYTQIAK [522], |
| | NVPPGLDEYNPFSDSR [560] |

e-Lung cancer

| | |
|---|---|
| 31584 | KVHGLYR [441], NVPPGLDEYNPFSDSR [560] |
| 31835 | EMQNLSQHGR [211], NVPPGLDEYNPFSDSR [560] |
| 33009 | EMQNLSQHGR [211], NVPPGLDEYNPFSDSR [560], |
| | QEELER [566] |
| 33422 | EHALAQAELLK [190], EMQNLSQHGR [211], |
| | MPNVPNTQPAIMKPTEEHPAYTQIAK [522], |
| | NVPPGLDEYNPFSDSR [560], RQEELER [612] |
| 33847 | EMQNLSQHGR [211], NVPPGLDEYNPFSDSR [560] |
| 34135 | AQQEFATGVMSNK [84], EMQNLSQHGR [211], |
| | NVPPGLDEYNPFSDSR [560] |
| 35643 | EMQNLSQHGR [211], NVPPGLDEYNPFSDSR [560], |
| | TPPPGGVK [721], TVQTAAANAASTAASSAAQNAFK [737] |

f-Lymphoid leukaemia, unspecified

| | |
|---|---|
| 28206 | EHALAQAELLK [190], EMQNLSQHGR [211], |
| | NVPPGLDEYNPFSDSR [560] |

g-Pancreatic cancer

| | |
|---|---|
| 26742 | EHALAQAELLK [190], NVPPGLDEYNPFSDSR [560] |
| 31738 | EHALAQAELLK [190], EMQNLSQHGR [211], |
| | NVPPGLDEYNPFSDSR [560] |
| 32245 | EHALAQAELLK [190], EMQNLSQHGR [211], |
| | NVPPGLDEYNPFSDSR [560] |
| 34059 | DPSVTQVTR [149], EMQNLSQHGR [211], KVHGLYR [441], |
| | NVPPGLDEYNPFSDSR [560], TVQTAAANAASTAASSAAQNAFK [737] |
| 34270 | DPSVTQVTR [149], EMQNLSQHGR [211], |
| | NVPPGLDEYNPFSDSR [560] |
| 36671 | EHALAQAELLK [190], EMQNLSQHGR [211], KAAELDRR [406], |
| | NVPPGLDEYNPFSDSR [560], RQEELER [612] |
| 36974 | DPSVTQVTR [149], EHALAQAELLK [190], EMQNLSQHGR [211], |
| | NVPPGLDEYNPFSDSR [560], RQEELER ]612] |

h-Renal cell cancer

| | |
|---|---|
| 30318 | EHALAQAELLK [190], EMQNLSQHGR [211], |
| | NVPPGLDEYNPFSDSR [560] |
| 33762 | EHALAQAELLK [190], EMQNLSQHGR [211], KVHGLYR [441], |
| | NVPPGLDEYNPFSDSR [560] |

i-Retinoblastoma

| | |
|---|---|
| 30816 | EHALAQAELLK [190], EMQNLSQHGR [211], |
| | NVPPGLDEYNPFSDSR [560], RQEELER [612], |
| | TVQTAAANAASTAASSAAQNAFK [737], VHGLYR [766] |

OGTA089

TABLE 16

| | | |
|---|---|---|
| MW | | |
| (Da) | Subfractionation | Tryptics identified [SEQ ID No] |

a-Breast cancer

| | |
|---|---|
| 110512 | ADFLIFR [59], EEINKPPIAK [176], ELTLPVDSTTLDGSK [207], |
| | GETYTYDWQLITHPR [293], VVEMQGVR [809] |

b-Lung cancer

| | |
|---|---|
| 72110 | GETYTYDWQLITHPR [293], IQPYTEQSTK [391], |
| | VNDSNELGGLTTSGSAEVHK [786] |

c-Ovarian cancer

| | |
|---|---|
| 77125 | GETYTYDWQLITHPR [293], NLQSQSSVNVIVK [544] |
| 89348 | ADFLIFR [59], EEINKPPIAK [176], GETYTYDWQLITHPR [293], |
| | ILDATDQESLELKPTSR [374], IQPYTEQSTK [391], |
| | LTPGLYEFK [497], MVFFVQNEPPHQIFK [528], |
| | NVSVQPEISEGLATTPSTQQVK [562], THSSNSMLVFLK [699] |

OGTA091

TABLE 17

| | | |
|---|---|---|
| MW | | |
| (Da) | Subfractionation | Tryptics identified [SEQ ID No] |

a-Breast cancer

| | | |
|---|---|---|
| 24993 | Vesicles | NLVDPFVEVSFAGK [547] |
| 114969 | | TDVHYR [686] |
| 120658 | | TDVHYR [686] |
| 124535 | PNGase F | GEGVAYR [291] |
| | Deglycosylation | |
| 142847 | | GEGVAYR [291] |
| 164389 | | GEGVAYR [291], NLVDPFVEVSFAGK [547] |
| 165165 | | GEGVAYR [291], NLVDPFVEVSFAGK [547] |
| 171988 | | GEGVAYR [291], NLVDPFVEVSFAGK [547], TDVHYR [686] |
| 176984 | | GEGVAYR [291], TDVHYR [686] |
| 179325 | | GEGVAYR [291], NLVDPFVEVSFAGK [547] |
| 210126 | PNGase F | TDVHYR [686] |
| | Deglycosylation | |
| 230530 | PNGase F | TDVHYR [686] |
| | Deglycosylation | |

b-Cervical cancer

GEGVAYR [291], TDVHYR [686]
GEGVAYR [291], TDVHYR [686]
TDVHYR [686]

c-Chronic lymphocytic leukaemia

| | |
|---|---|
| 223568 | FSDVTGK [272], TDVHYR [686] |

d-Gastric cancer

| | |
|---|---|
| 95956 | TDVHYR [686] |
| 125740 | TDVHYR [686] |
| 129831 | NLVDPFVEVSFAGK [547] |
| 138914 | GEGVAYR [291], NLVDPFVEVSFAGK [547], TDVHYR [686] |
| 143975 | GEGVAYR [291], TDVHYR [686] |

e-Hepatocellular carcmoma

| | |
|---|---|
| 98239 | GEGVAYR [291] |
| 102119 | GEGVAYR [291], NLVDPFVEVSFAGK [547] |
| 106345 | GEGVAYR [291], TDVHYR [686] |
| 107073 | GEGVAYR [291] |
| 110964 | GEGVAYR [291], TDVHYR [686] |
| 114377 | GEGVAYR [291] |
| 116037 | GEGVAYR [291], TDVHYR [686] |
| 118513 | GEGVAYR [291], NLVDPFVEVSFAGK [547], TDVHYR [686] |
| 121636 | FFASIGER [250], FHLEYR [254], GEGVAYR [291], |
| | IGETVVDLENR [367], IIDWDR [370], IPNPHLGPVEER [386], |
| | KTDAFRR [436], LLSDKPQDFQIR [469], QIFGFESNK [579], |
| | SLGGEGNFNWR [640], TDALLGEFR [681], TDVHYR [686], |
| | VEDLPADDILR [753], VQVIEGR [794], VTAAGQTK [800] |
| 123033 | NLVDPFVEVSFAGK [547], TDVHYR [686] |
| 127849 | GEGVAYR [291] |
| 128000 | TDVHYR [686] |
| 133487 | AVDEQGWEYSITIPPER [94], DLSQMEALK [139], |
| | DSFSDPYAIVSFLHQSQK [155], EKPAIHHIPGFEVQETSR [201], |
| | EVLATPSLSASFNAPLLDTK [234], FHLEYR [254], GEGVAYR [291], |
| | GSQPSGELLASFELIQR [320], HWVPAEK [348], |
| | IETQNQLLGIADR [363], IGETVVDLENR [367], |
| | ILDESEDTDLPYPPPQR [375], IPNPHLGPVEER [386], |
| | IYPLPEDPAIPMPPR [403], LPGGQWIYMSDNYTDVNGEK [477], |
| | LSVFAETYENETK [491], MDVGTIYR [511], MFELTCTLPLEK [515], |
| | MYYTHRRR [533], RMEPLEK [607], RPDTSFLWFTSPYK [608], |
| | SLGGEGNFNWR [640], TDALLGEFR [681], TDVHYR [686], |
| | TSLCVLGPGDEAPLERK [729], VEDLPADDILR [753], |
| | VPAHQVLFSR [787], VQVIEGR [794], VVFQIWDNDK [810], |
| | WEDEEWSTDLNR [824], WLLLSDPDDFSAGAR [827] |
| 139587 | AVDEQGWEYSITIPPER [94], DLSQMEALK [139], |
| | DLSQMEALKR [140], FFASIGER [250], |
| | ILDESEDTDLPYPPPQR [375], KNPNFDICTLFMEVMLPR [430], |
| | LPGGQWIYMSDNYTDVNGEK [477], MFELTCTLPLEK [515], |
| | NPNFDICTLFMEVMLPR [551], QFHQLAAQGPQECLVR +570+, |
| | RDLSQMEALK [599], RMEPLEK [607], |
| | TANPQWNQNITLPAMFPSMCEK [670], TDAFRRR [680], |
| | TDALLGEFR [681], TQEETEDPSVIGEFK [724], |
| | TSLCVLGPGDEAPLER [728], VEDLPADDILR [753], |
| | VVFQIWDNDK [810] |
| 192580 | TDVHYR [686] |
| 200565 | GEGVAYR [291], TDVHYR [686] |
| 209214 | TDVHYR [686] |

f- Lung cancer

| | |
|---|---|
| 195217 | GEGVAYR [291], TDVHYR [686] |
| 202986 | GEGVAYR [291], KNLVDPFVEVSFAGK [429], TDVHYR [686] |
| 211353 | GEGVAYR [291], KNLVDPFVEVSFAGK [429], TDVHYR [686] |
| 220388 | GEGVAYR [291], KNLVDPFVEVSFAGK [429], TDVHYR [686] |
| 230173 | EYSIEEIEAGR [241], ILDESEDTDLPYPPPQR [375], |
| | IPNPHLGPVEER [386], MEPLEK [513], RMEPLEK [607], |
| | SLGGEGNFNWR [640], TDVHYR [686] |

g-Melanoma

| | |
|---|---|
| 20629 | FSDVTGK [272], NLVDPFVEVSFAGK [547] |

h-Osteosarcoma

| | |
|---|---|
| 151810 | NLVDPFVEVSFAGK [547] |
| 159457 | TDVHYR [686] |
| 181827 | NLVDPFVEVSFAGK [547] |

i-Ovarian cancer

| | |
|---|---|
| 70973 | KNLVDPFVEVSFAGK [429] |
| 74967 | KNLVDPFVEVSFAGK [429] |
| 110436 | NLVDPFVEVSFAGK [547] |
| 115528 | NLVDPFVEVSFAGK [547] |
| 127159 | GEGVAYR [291] |

j-Pancreatic cancer

| | |
|---|---|
| 55323 | TDVHYR [686] |
| 70820 | GEGVAYR [291] |
| 88202 | FSDVTGK [272], KNLVDPFVEVSFAGK [429] |
| 100099 | GEGVAYR [291] |
| 132648 | TDVHYR [686] |
| 133778 | TDVHYR [686] |
| 142218 | GEGVAYR [291] |
| 142690 | NLVDPFVEVSFAGK [547] |
| 154726 | GEGVAYR [291], NLVDPFVEVSFAGK [547] |
| 158113 | GEGVAYR [291] |
| 168430 | GEGVAYR [291], TDVHYR [686] |
| 174222 | NLVDPFVEVSFAGK [547] |
| 184505 | FSDVTGK [272], TDVHYR [686] |
| 189061 | KNLVDPFVEVSFAGK [429] |
| 192682 | TDVHYR [686] |
| 208929 | GEGVAYR [291], NLVDPFVEVSFAGK [547], TDVHYR [686] |
| 216690 | GEGVAYR [291], NLVDPFVEVSFAGK [547], TDVHYR [686] |
| 217481 | TDVHYR [686] |
| 222673 | TDVHYR [686] |
| 229012 | GEGVAYR [291], TDVHYR [686] |
| 230839 | GEGVAYR [291], TDVHYR [686] |
| 261976 | TDVHYR [686] |

k-Prostate cancer

GEGVAYR [291], TDVHYR [686]
TDVHYR [686]

l-Renal cell cancer

| | |
|---|---|
| 62841 | TDVHYR [686] |
| 83490 | GEGVAYR [291] |
| 122161 | FSDVTGK [272], GEGVAYR [291] |
| 126235 | GEGVAYR [291] |
| 135291 | GEGVAYR [291] |
| 143822 | GEGVAYR [291], TDVHYR [686] |
| 148249 | TDVHYR [686] |
| 151721 | GEGVAYR [291], TDVHYR [686] |
| 158158 | ALGRPGPPFNITPR [76], AVDEQGWEYSITIPPER [94], |
| | CIIWNTR [104], DLAAMDK [132], DLSQMEALK [139], |
| | EFNQFAEGK [185], EKPAIHHIPGFEVQETSR [201], |
| | EYSIEEIEAGR [241], FHLEYR [254], FSFDDFLGSLQLDLNR [273], |
| | GEGVAYR [291], GWMIGFEEHK [329], |
| | IETQNQLLGIADR [363], IGETVVDLENR [367], IIDWDR [370], |
| | ILDESEDTDLPYPPPQR [375], IYPLPEDPAIPMPPR [403], |
| | KLPSRPPPHYPGIK [426], LLSDKPQDFQIR [469], MDVGTIYR [511], |
| | MYYTHR [531], MYYTHRR [532], RMEPLEK [607], |
| | SLGGEGNFNWR [640], SYQLANISSPSLVVECGGQTVQSCVIR [667], |
| | TDALLGEFR [681], TQEETEDPSVIGEFK [724], |
| | VEDLPADDILR [753], VQVIEGR [794], WEDEEWSTDLNR [824], |
| | WLLLSDPDDFSAGAR [827] |
| 200334 | NLVDPFVEVSFAGK [547] |
| 221217 | GEGVAYR [291], NLVDPFVEVSFAGK [547], TDVHYR [686] |
| 233732 | TDVHYR [686] |

OGTA098

TABLE 18

| | | |
|---|---|---|
| MW | | |
| (Da) | Subfractionation | Tryptics identified [SEQ ID No] |

a-Breast cancer

| | |
|---|---|
| 49867 | VVPSFLPVDQGGSLVGR [812] |
| 50576 | DGNGEVTDKPVK [123], DMAGAQAAAVALNEEFLR [142], |
| | DNWISVDSVTSEIK [148], DTGEIYTTSVTLDR [159], |
| | EETPFFLLTGYALDAR [182], NGVGGMAK [536], |
| | QESTSVLLQQSEKK [569], VTQEIVTER [806], |
| | VVPSFLPVDQGGSLVGR [812], |
| | VYAPASTLVDQPYANEGTVVVTER [816], YVQNGTYTVK [876] |
| 115607 | ATQFTGATGAIMTTETTK [93], DGNGEVTDKPVK [123], |
| | FLDDLGLK [257], IVAISEDYPR [400], QESTSVLLQQSEK [568], |
| | VVPSFLPVDQGGSLVGR [812] |

b-Cervical cancer

| | | |
|---|---|---|
| 82244 | MonoQ Aqueous | DTGEIYTTSVTLDR [159], EEHSSYTLTVEAR [174], |
| | | IVAISEDYPR [400] |

c-Hepatocellular carcmoma

| | |
|---|---|
| 83238 | ATQFTGATGAIMTTETTK [93], GNNVEKPLELR [304], |
| | INATDADEPNTLNSK [381], IVAISEDYPR [400], |
| | QESTSVLLQQSEK [568] |
| 112208 | ATQFTGATGAIMTTETTK [93], DNWISVDSVTSEIK [148], |
| | DTGEIYTTSVTLDR [159], FLDDLGLK [257], GNNVEKPLELR [304], |
| | GQIIGNFQAFDEDTGLPAHAR [311], IVAISEDYPR [400], |
| | VATPLPDPMASR [746], VLEGMVEENQVNVEVTR [778], |
| | VTQEIVTER [806], VVPSFLPVDQGGSLVGR [812], WEEHR [825] |
| 134896 | ATQFTGATGAIMTTETTK [93] |

d-Lung cancer

ATQFTGATGAIMTTETTK [93], DTGEIYTTSVTLDR [159],
EEHSSYTLTVEAR [174], FLDDLGLK [257], GNNVEKPLELR [304],
GQIIGNFQAFDEDTGLPAHAR [311],
INATDADEPNTLNSK [381], IVAISEDYPR [400], LPDFESR [476],
QESTSVLLQQSEK [568], VATPLPDPMASR [746],
VLEGMVEENQVNVEVTR [778], VTQEIVTER [806],
VVPSFLPVDQGGSLVGR [812]
DNWISVDSVTSEIK [148], DTGEIYTTSVTLDR [159],
EEHSSYTLTVEAR [174], GNNVEKPLELR [304],
GQIIGNFQAFDEDTGLPAHAR [311], INATDADEPNTLNSK [381],
IVAISEDYPR [400], LPDFESR [476],
VLAPASTLQSSYQIPTENSMTAR [772],
VLEGMVEENQVNVEVTR [778], VVPSFLPVDQGGSLVGR [812],
VYAPASTLVDQPYANEGTVVVTER [816], YKPTPIPIK [858]

e-Osteosarcoma

| | |
|---|---|
| 24158 | GITEPPFGIFVFNK [301] |
| 41065 | DNWISVDSVTSEIK [148], IVAISEDYPR [400], |
| | VLEGMVEENQVNVEVTR [778] |

f-Pancreatic cancer

| | |
|---|---|
| 47024 | ATQFTGATGAIMTTETTK [93], DGNGEVTDKPVK [123], |
| | EGEDLSKK [187], ESFLAPSSGVQPTLAMPNIAVGQNVTVTER |
| | [223], GSSSASIVK [322], VATPLPDPMASR [746], VTQEIVTER |
| | [806], VVPSFLPVDQGGSLVGR [812], |
| | VYAPASTLVDQPYANEGTVVVTER [816] |
| 47767 | ATQFTGATGAIMTTETTK [93], DTGEIYTTSVTLDR [159], |
| | ESFLAPSSGVQPTLAMPNIAVGQNVTVTER [223], GSSSASIVK |
| | [322], VATPLPDPMASR [746], VLAPASTLQSSYQIPTENSMTAR |
| | [772], VTQEIVTER [806], VVPSFLPVDQGGSLVGR [812], |
| | VYAPASTLVDQPYANEGTVVVTER [816], WEEHR [825] |
| 48531 | ATQFTGATGAIMTTETTK [93], DGNGEVTDKPVK [123], |
| | VTQEIVTER [806], VVPSFLPVDQGGSLVGR [812], |
| | VYAPASTLVDQPYANEGTVVVTER [816], WEEHR [825] |
| 57915 | ATQFTGATGAIMTTETTK [93], DGNGEVTDKPVK [123], |
| | DMAGAQAAAVALNEEFLR [142], DTGELNVTSILDR [160], |
| | EEHSSYTLTVEAR [174], EETPFFLLTGYALDAR [182], |
| | ESFLAPSSGVQPTLAMPNIAVGQNVTVTER [223], |
| | GNNVEKPLELR [304], INATDADEPNTLNSK [381], |
| | SSVISIYVSESMDR [653], VTQEIVTER [806], |
| | VVPSFLPVDQGGSLVGR [812], |
| | VYAPASTLVDQPYANEGTVVVTER [816], WEEHR [825] |
| 58846 | ATQFTGATGAIMTTETTK [93], DGNGEVTDKPVK [123], |
| | EGEDLSKK [187], ESFLAPSSGVQPTLAMPNIAVGQNVTVTER |
| | [223], IHSDLAEER [369], INATDADEPNTLNSK [381], |
| | VTQEIVTER [806], VVPSFLPVDQGGSLVGR [812], |
| | VYAPASTLVDQPYANEGTVVVTER [816], WEEHR [825] |
| 119656 | DGNGEVTDKPVK [123], DMAGAQAAAVALNEEFLR [142], |
| | GNNVEKPLELR [304], INATDADEPNTLNSK [381], |
| | IVAISEDYPR [400], SEIQFLISDNQGFSCPEK [628], |
| | SSVISIYVSESMDR [653] |
| 134980 | ATQFTGATGAIMTTETTK [93], DNWISVDSVTSEIK [148], |
| | DTGEIYTTSVTLDR [159], EEHSSYTLTVEAR +174], |
| | GNNVEKPLELR [304], GQIIGNFQAFDEDTGLPAHAR [311], |
| | VTQEIVTER [806], VYAPASTLVDQPYANEGTVVVTER [816] |
| 142690 | ATQFTGATGAIMTTETTK [93], DTGEIYTTSVTLDR [159], |
| | EEHSSYTLTVEAR [174], EETPFFLLTGYALDAR [182], |
| | GNNVEKPLELR [304], GQIIGNFQAFDEDTGLPAHAR [311], |
| | QESTSVLLQQSEK [568], VLEGMVEENQVNVEVTR [778] |

g-Prostate cancer

DTGEIYTTSVTLDR [159], FLDDLGLK [257], VATPLPDPMASR [746]

h-Renal cell cancer

| | |
|---|---|
| 111421 | GNNVEKPLELR [304], IVAISEDYPR [400], LPDFESR [476], |
| | VTQEIVTER [806] |

OGTA101

TABLE 19

| | | |
|---|---|---|
| MW | | |
| (Da) | Subfractionation | Tryptics identified [SEQ ID No] |

a-Breast cancer

| | | |
|---|---|---|
| 24841 | Vesicles | QFQFTDWPEQGVPK [572], YQYFVVDPMAEYNMPQYILR [869] |
| 24993 | Vesicles | AALEYLGSFDHYAT [51], QFQFTDWPEQGVPK [572], |
| | | YQYFVVDPMAEYNMPQYILR [869] |
| 55008 | | GVEGSDYINASFIDGYR [325], YQYFVVDPMAEYNMPQYILR [869] |
| 55921 | | AALEYLGSFDHYAT [51], QFQFTDWPEQGVPK [572] |
| 56324 | | QHGQIR [578] |
| 56872 | | AEPESETSILLSWTPPR [63], EIPSHHPTDPVELR [198], |
| | | FIKPWESPDEMELDELLK [255], GFPTIDMGPQLK [295], |
| | | GPPSEPVLTQTSEQAPSSAPR [309], GVEGSDYINASFIDGYR |
| | | [325], LTQIETGENVTGMELEFK [498], LVNIMPYESTR [503], |
| | | MLWEHNSTIVVMLTK [520], MVEEVDGR [527], SYSFVLTNR |
| | | [668], TATMLCAASGNPDPEITWFK [675], TNEDVPSGPPRK |
| | | [709], TSVLLSWEIPENYNSAMPFK [731], VGFGEEMVK [763] |
| 105325 | | TGCFIVIDAMLER [692] |

b-Cervical cancer

| | | |
|---|---|---|
| 118858 | Integral | QHGQIR [578], WTEYR [836] |
| 124604 | Integral | WTEYR [836] |

c-Colorectal cancer

| | |
|---|---|
| Heparin Binding | YSAPANLYVR [870] |

d-Hepatocellular carcinoma

| | |
|---|---|
| 72397 | DMEPLTTLEFSEK [143], GFPTIDMGPQLK [295], |
| | GPGPYSPSVQFR [306], SGEGFIDFIGQVHK [632], |
| | TATMLCAASGNPDPEITWFK [675], YSAPANLYVR [870], |
| | YANVIAYDHSR [840] |

e-Lung cancer

| | |
|---|---|
| | YSAPANLYVR [870] |
| 77419 | FIKPWESPDEMELDELLK [255], GFPTIDMGPQLK [295], |
| | GVEGSDYINASFIDGYR [325], LTQIETGENVTGMELEFK [498], |
| | QFQFTDWPEQGVPK [572], SDTIANYELVYK [625], |
| | TATMLCAASGNPDPEITWFK [675], TVDIYGHVTLMR [736], |
| | VEVEAVNSTSVK [755], YEGVVDIFQTVK [843] |
| 78771 | YANVIAYDHSR [840] |

f-Osteosarcoma

| | |
|---|---|
| 63624 | QHGQIR [578], YQYFVVDPMAEYNMPQYILR [869] |

g-Pancreatic cancer

| | |
|---|---|
| 74307 | DFLPVDTSNNNGR [118], EIPSHHPTDPVELRR [199], |
| | EQFGQDGPISVHCSAGVGR [219], GFPTIDMGPQLK [295], |
| | GNSAGGLQHR [305], GVEGSDYINASFIDGYR [325], |
| | KRAESDSR [434], LTQIETGENVTGMELEFK [498], |
| | LVNIMPYESTR [503], RAESDSRK [595], SDTIANYELVYK |
| | [625], SGEGFIDFIGQVHK [632], TGCFIVIDAMLER [692], |
| | TVDIYGHVTLMR [736], YANVIAYDHSR [840], |
| | YSVAGLSPYSDYEFR [873] |
| 115291 | TGCFIVIDAMLER [692], YQYFVVDPMAEYNMPQYILR [869], |
| | YSAPANLYVR [870] |
| 124451 | TGCFIVIDAMLER [692], YSAPANLYVR [870] |
| 126215 | YSAPANLYVR [870] |
| 129890 | YSAPANLYVR [870] |

OGTA104

TABLE 20

| | | |
|---|---|---|
| MW | | |
| (Da) | Subfractionation | Tryptics identified [SEQ ID No] |

a-Breast cancer

| | |
|---|---|
| 78181 | EVPIYANR [236], FAVPTYAAK [244], QPQQFPSRPPPPQPK [586] |

b-Hepatocellular carcmoma

| | |
|---|---|
| 63302 | DEEEEPPSMTQLLR [113], DLLPEDFVVYTYNK [134], |
| | EVPIYANR [236], KGFGGTAGMAFVGTVCSR [419], QPGSVPR |
| | [584], QPQQFPSRPPPPQPK [586], RDQLWR [600] |

c-Ovarian cancer

| | |
|---|---|
| 49732 | EVPIYANR [236], KGFGGTAGMAFVGTVCSR [419], |
| | QPQQFPSRPPPPQPK [586] |
| 50782 | DQLWR [152], FAVPTYAAK [244], HVSPVTPPR [346], |
| | KGFGGTAGMAFVGTVCSR [419], QPQQFPSRPPPPQPK [586], |
| | SYFRK [666] |
| 54191 | FAVPTYAAK [244], FLPGGTLCR [260], FPSGTLR [264], |
| | QPQQFPSRPPPPQPK [586], SYFRK [666] |

d-Pancreatic cancer

| | |
|---|---|
| 73243 | EEMILLANYLDSMYIMLNIR [179], EVPIYANR [236], |
| | HVSPVTPPR [346], KGFGGTAGMAFVGTVCSR [419], |
| | QPQQFPSRPPPPQPK [586] |

OGTA106

TABLE 21

| | | |
|---|---|---|
| MW (Da) | Subfractionation | Tryptics identified [SEQ ID No] |

a - Cervical cancer

| | | |
|---|---|---|
| 61458 | MonoQ Aqueous | SLSGVGAGGGPTPR [642] |

b - Melanoma

| | |
|---|---|
| 75458 | CHIQASGRPQCSCMPGWTGEQCQLR [103], |
| | EQTPLFLAAR [221], ETDSLSAGFVVVMGVDLSR |
| | [229], GPRPNPAIMR [310], |
| | GSPQLDCGPPALQEMPINQGGEGKK [318], |
| | TLSAGAGPR [705], TPLHAAVAADAR [717] |

c - Pancreatic cancer

| | |
|---|---|
| 88318 | EQTPLFLAAR [221] |

OGTA112

TABLE 22

| | | |
|---|---|---|
| MW (Da) | Subfractionation | Tryptics identified [SEQ ID No] |

a - Breast cancer

| | |
|---|---|
| Triton X114 | ETCVDLHLPR [228], FMFDLFQQFR [261], |
| | FYQTSVESTDFANAPEESR [280], |
| | FYQTSVESTDFANAPEESRKK [281], |
| | KFYQTSVESTDFANAPEESRK [416], KINSWVESQTNEK [420], |
| | LMEWTSLQNMR [470], QYNSFNFALLEDVQAK [594], |
| | TNSILFYGR [713], TYQFLQEYLDAIK [741], |
| | VLHFDQVTENTTEK [781] |

b - Cervical cancer

| | | |
|---|---|---|
| 133844 | MonoQ Aqueous | FMFDLFQQFRK [262], LMEWTSLQNMR [470], SVQMMR [665], |
| | | TMGMVNIFNGDADLSGMTWSHGLSVSK [706], VLEIPYK [779] |

c - Chronic lymphocytic leukaemia

| | |
|---|---|
| 34501 | ETCVDLHLPR [228], FMFDLFQQFR [261] |

d - Hepatocellular carcinoma

| | |
|---|---|
| 26010 | AATYHVDR [54], ETCVDLHLPR [228], FMFDLFQQFR [261], |
| | INSWVESQTNEK [384], LMEWTSLQNMR [470], |
| | QYNSFNFALLEDVQAK [594], TNSILFYGR [713], |
| | VLHFDQVTENTTEK [781] |

e - Pancreatic cancer

| | |
|---|---|
| 37971 | ETCVDLHLPR [228], FMFDLFQQFR [261], FMFDLFQQFRK |
| | [262], INSWVESQTNEK [384], LMEWTSLQNMR [470], |
| | QYNSFNFALLEDVQAK [594], SVQMMR [665], TNSILFYGR |
| | [713], VLHFDQVTENTTEK [781] |
| 77017 | ETCVDLHLPR [228], FMFDLFQQFR [261], INSWVESQTNEK |
| | [384], LMEWTSLQNMR [470], QYNSFNFALLEDVQAK [594], |
| | SVQMMR [665] |

f - Renal cell cancer

| | |
|---|---|
| 34256 | INSWVESQTNEK [384], LMEWTSLQNMR [470], SVQMMR |
| | [665], TNSILFYGR [713], VLHFDQVTENTTEK [781] |

OGTA113

TABLE 23
Pancreatic cancer

| MW (Da) | Subfractionation | Tryptics identified [SEQ ID No] |
|---|---|---|
| 32378 | | LEDPHVDIIR [449], LVHIIGALR [500], RGDFFYHSENPK [603], |
| | | SEIINSK [627], TSSQPGFLER [730], YSYNTEWR [874] |
| 32754 | | LEDPHVDIIR [449], LVHIIGALR [500], RGDFFYHSENPK [603], |
| | | TSSQPGFLER [730], YPEVGDLR [865], YSYNTEWR [874] |
| 42556 | | HVEMYQWVETEESR [345], LEDPHVDIIR [449], |
| | | RGDFFYHSENPK [603], TSSQPGFLER [730], YPEVGDLR [865], |
| | | YSYNTEWR [874] |
| 43294 | | HVEMYQWVETEESR [345], LEDPHVDIIR [449], LVHIIGALR |
| | | [500], RGDFFYHSENPK [603], TSSQPGFLER [730], YSYNTEWR |
| | | [874] |

OGTA119

TABLE 24

| | | |
|---|---|---|
| MW (Da) | Subfractionation | Tryptics identified [SEQ ID No] |

a - B-cell non-Hodgkin's lymphoma

| | |
|---|---|
| 115552 | AQPPEAGPQGLHDLGR [83], DEQHQCSLGNLK [115], |
| | ERPPDHQHSAQVK [222], GANTHLSTFSFTK [285], |
| | VGNQIFQSR [765] |

b - Breast cancer

| | |
|---|---|
| | AEWLNK [64] |
| 70772 | ALALLEDEER [75], AQPPEAGPQGLHDLGR [83], |
| | FQLSNSGPNSTIK [268], ISSNPNPVVQMSVGHK [393], |
| | NLIAFSEDGSDPYVR [542], VGNQIFQSR [765] |
| 98013 | AEWLNK [64] |
| 121313 | AEWLNK [64] |

c - Colorectal cancer

| | | |
|---|---|---|
| 96268 | Nucleotide Binding | ALALLEDEER [75], AQPPEAGPQGLHDLGR [83], |
| | | ERPPDHQHSAQVK [222], FQLSNSGPNSTIK [268], |
| | | LEWLTLMPNASNLDK [457], NLIAFSEDGSDPYVR [542], |
| | | RQDLEVEVR [611], TNEPVWEENFTFFIHNPK [710] |

d - Gastric cancer

| | |
|---|---|
| 56270 | VDVGQQPLR [750] |

e - Hepatocellular carcmoma

| | |
|---|---|
| 59358 | ALALLEDEER [75], ERPPDHQHSAQVK [222] |
| 60649 | ALALLEDEER [75], AQPPEAGPQGLHDLGR [83], RQDLEVEVR |
| | [611], VDVGQQPLR [750], VGNQIFQSR [765] |
| 73339 | AEWLNK [64], AQPPEAGPQGLHDLGR [83], |
| | ERPPDHQHSAQVK [222], NLIAFSEDGSDPYVR [542], |
| | SDPYGIIR [622], VGNQIFQSR [765], VPLSQLLTSEDMTVSQR |
| | [792] |
| 84442 | AEWLNK [64] |
| 87814 | AQPPEAGPQGLHDLGR [83], DEQHQCSLGNLK [115], |
| | ERPPDHQHSAQVK [222], FQLSNSGPNSTIK [268], |
| | HMWPFICQFIEK [339], IHFIEAQDLQGK [368], |
| | NLIAFSEDGSDPYVR [542], TNEPVWEENFTFFIHNPK [710], |
| | VDVGQQPLR [750] |
| 98646 | AEWLNK [64] |

f - Lung cancer

| | |
|---|---|
| 92181 | AQPPEAGPQGLHDLGR [83], ERPPDHQHSAQVK [222], |
| | HMWPFICQFIEK [339], ITVPLVSEVQIAQLR [398] |

g - Lymphoid leukaemia, unspecified

| | |
|---|---|
| 85026 | ETIEPAVR [230], FQLSNSGPNSTIK [268], |
| | GEGPEAGAGGAGGR [290], RQDLEVEVR [611], VDVGQQPLR |
| | [750] |
| 86516 | AQPPEAGPQGLHDLGR [83], ERPPDHQHSAQVK [222], |
| | GEGPEAGAGGAGGR [290], HMWPFICQFIEK [339], |
| | IHFIEAQDLQGK [368], ISSNPNPVVQMSVGHK [393], |
| | NLIAFSEDGSDPYVR [542], RQDLEVEVR [611], VDVGQQPLR |
| | [750], VLVALASEELAK [784] |

h - Neuroblastoma

| | |
|---|---|
| Sodium Carbonate | AEWLNK [64] |
| Scrub | |

i - Osteosarcoma

| | |
|---|---|
| 73285 | AEWLNK [64] |

j - Pancreatic cancer

| | |
|---|---|
| 80573 | AQPPEAGPQGLHDLGR [83], ERPPDHQHSAQVK [222], |
| | GEGPEAGAGGAGGR [290], HMWPFICQFIEK [339], |
| | IHFIEAQDLQGK [368], NLIAFSEDGSDPYVR [542], |
| | SDPYGIIR [622], VGNQIFQSR [765], VYTENVDK [819] |
| 93931 | ALALLEDEER [75], DEQHQCSLGNLK [115], |
| | ERPPDHQHSAQVK [222], GEGPEAGAGGAGGR [290], |
| | ISSNPNPVVQMSVGHK [393], VPLSQLLTSEDMTVSQR [792] |

k - Prostate cancer

| | |
|---|---|
| Digitonin Insoluble, | AEWLNK [64] |
| Triton X114, | |
| Aqueous | |

l - Renal cell cancer

| | |
|---|---|
| 76269 | AEWLNK [64], AQPPEAGPQGLHDLGR [83], |
| | ERPPDHQHSAQVK [222], HMWPFICQFIEK [339], SIQIHGTMR |
| | [638], TNEPVWEENFTFFIHNPK [710], VDVGQQPLR [750], |
| | VYTENVDKR [820] |

OGTA124

TABLE 25

| | | |
|---|---|---|
| MW (Da) | Subfractionation | Tryptics identified [SEQ ID No] |

a - Hepatocellular carcmoma

| | |
|---|---|
| 98003 | DLAVFLYHLR [133], EGLAHLIQSCGLGGMR [188], |
| | ESSPFLSPLEASR [227], EWGDGIR [240], HGLPSADAPSLK |
| | [334], HNSVVLGWPYGWR [340], LDEDLHVK [446], |
| | LEEGPPHTK [451], LVLLNMPGPPR [501], MPHFTVVPVDGPR |
| | [521], NIAFYPSNHER [539], NLALFEEELDIRPK [541], |
| | SIFDPPVFPVCMLGNR [636] |
| 116154 | ESSPFLSPLEASR [227], EWGDGIR [240], HGLPSADAPSLK |
| | [334], LEEGPPHTK [451], NIAFYPSNHER [539], TPNWRPR |
| | [720], YIEYQGAEK [855] |
| 126143 | EHEEAESGEGTRRR [192], ESSPFLSPLEASR [227], EWGDGIR |
| | [240], HNSVVLGWPYGWR [340], LVSYTNLTQGAK [504], |
| | MPHFTVVPVDGPR [521], NSEGDENYMEFLEVLTEGLER [553], |
| | TFIDTVR [689], YMTETWDPSHAPDNFR [863] |
| 137186 | AELDDSDGHGNHR [62], EVITIYS [233], LVSYTNLTQGAK |
| | [504], MPHFTVVPVDGPR [521], NIAFYPSNHER [539] |

b - Lung cancer

| | |
|---|---|
| 153348 | DQFDICAK [151], EHEEAESGEGTR [191], EHEEAESGEGTRRR |
| | [192], ESSPFLSPLEASR [227], EVITIYS [233], |
| | LVLLNMPGPPR [501], LVSYTNLTQGAK [504] |

c - Ovarian cancer

| | |
|---|---|
| 69811 | ESSPFLSPLEASR [227], EVITIYS [233], IQMTWTR [389], |
| | LNEVIVTR [472], MHTAVK [517], NIAFYPSNHER [539], |
| | NSEGDENYMEFLEVLTEGLER [553], TPNWRPR [720] |
| 74557 | ESSPFLSPLEASR [227], GIDYYDR [300], NIAFYPSNHER |
| | [539], TFIDTVR [689], TPNWRPR [720] |

d - Pancreatic cancer

| | |
|---|---|
| 137609 | ELVHIKPDQSNVR [209], ESSPFLSPLEASR [227], |
| | HNSVVLGWPYGWR [340], LEEGPPHTK [451], |
| | LVSYTNLTQGAK [504], NIAFYPSNHER [539], |
| | SIFDPPVFPVCMLGNR [636] |

e - Renal cell cancer

| | |
|---|---|
| 135833 | HNSVVLGWPYGWR [340], IFTVAQMDDNSIQMK [364], |
| | LVSYTNLTQGAK [504], MPHFTVVPVDGPR [521], |
| | NIAFYPSNHER [539] |
| 154919 | ESSPFLSPLEASR [227], HNSVVLGWPYGWR [340], |
| | IFTVAQMDDNSIQMKK [365], LVSYTNLTQGAK [504], |
| | SIFDPPVFPVCMLGNR [636] |

OGTA126

TABLE 26

| | | |
|---|---|---|
| MW (Da) | Subfractionation | Tryptics identified [SEQ ID No] |

a - Breast cancer

| | |
|---|---|
| 105325 | AFMIIQEQR [66], GAEAFEIALPR [282], GLPSLTSVSWNISVPR |
| | [302], IYVVDLSNER [404], MALHLPWFHPR [508] |

b - Colorectal cancer

| | |
|---|---|
| 104028 | ASVSFLNFNLSNCER [87], MALHLPWFHPR [508], |
| | VEYYIPGSTTNPEVFK [757] |

c - Hepatocellular carcinoma

| | |
|---|---|
| 82606 | AFMIIQEQR [66], ASVSFLNFNLSNCER [87], EEGVFTVTPDTK |
| | [172], LATEEPPPR [444], LWMNVEK [505], |
| | QIGPGESCPDGVTHSISGR [580], TCSSNLTLTSGSK [679] |
| 86381 | AFMIIQEQR [66], LATEEPPPR [444] |
| 88432 | AFMIIQEQR [66], ASVSFLNFNLSNCER [87], IYVVDLSNER |
| | [404], LATEEPPPR [444], LWMNVEK [505], MALHLPWFHPR |
| | [508], NVSGFSIANR [561], QIGPGESCPDGVTHSISGR [580] |
| 90607 | AFMIIQEQR [66], LATEEPPPR [444], NVSGFSIANR [561] |

d - Melanoma

| | |
|---|---|
| 81352 | DQVACLTFFK [153], IYVVDLSNER [404], KFVPGCFVCLESR |
| | [415] |
| 87990 | AFMIIQEQR [66], DQVACLTFFK [153], IYVVDLSNER [404], |
| | MALHLPWFHPR [508] |
| 102001 | AFMIIQEQR [66], ASVSFLNFNLSNCER [87], IYVVDLSNER |
| | [404], LATEEPPPR [444] |

e - Pancreatic cancer

| | |
|---|---|
| 57018 | QGLTVSFIPYFK [576], QIGPGESCPDGVTHSISGR [580], |
| | QPGNMAGNFNLSLQGCDQDAQSPGILR [583], |
| | VEYYIPGSTTNPEVFK [757[ |

f - Prostate cancer

AFMIIQEQR [66], EEGVFTVTPDTK [172], IDATVVR [353],
IYVVDLSNER [404], LWMNVEK [505], TPNWDR [719]
AFMIIQEQR [66], IYVVDLSNER [404], LATEEPPPR [444],
TPNWDR [719]
AFMIIQEQR [66], EEGVFTVTPDTK [172], IYVVDLSNER [404],
LWMNVEK [505], TPNWDR [719], VEYYIPGSTTNPEVFK [757]

g - Renal cell cancer

| | |
|---|---|
| 125611 | KFVPGCFVCLESR [415], LSLVLVPAQK [487], SGVVCQTGR |
| | [633] |

OGTA156

TABLE 27

| | | |
|---|---|---|
| MW (Da) | Subfractionation | Tryptics identified [SEQ ID No] |

a - Acute T - cell leukaemia

ADGISSTFSQR [61], ATGFPEPNPR [90], AYIFSIDEK [99],
SILQEENR [637], THQAFMR [697], TINFAR [701]
ADGISSTFSQR [61], ATGFPEPNPR [90], AYIFSIDEK [99],
DNQWLGVTLSR [147], LPTGGCYGVPPDLR [482], SILQEENR
[637], SQHTTEVVGGAPQHEQIGK [648], TINFAR [701]
ATGFPEPNPR [90], AYIFSIDEK [99], SILQEENR [637],
THQAFMR [697], TINFAR [701], VTVAIPLK [807]
ATGFPEPNPR [90], AYIFSIDEK [99], SILQEENR [637],
SQHTTEVVGGAPQHEQIGK [648], THQAFMR [697]
ATGFPEPNPR [90], AYIFSIDEK [99], SILQEENR [637]

b - B - cell non - Hodgkin's lymphoma

| | |
|---|---|
| 112390 | ADGISSTFSQR [61], ATGFPEPNPR [90], AYIFSIDEK [99], |
| | DNQWLGVTLSR [147], EASVHIQLEGRPSILEMDETSALK [167], |
| | LPVGLYFIK [484], NIFYIK [540], SDSAVLLR [624], SILQEENR |
| | [637], SQHTTEVVGGAPQHEQIGK [648], TINFAR [701], |
| | TYLAVGSMK [740] |
| 114990 | ADGISSTFSQR [61], ATGFPEPNPR [90], AYIFSIDEK [99], |
| | DNQWLGVTLSR [147], IAPCYQDYVK [351], KAESPPR [407], |
| | NIFYIK [540], SDSAVLLR [624], SILQEENR [637], |
| | SQHTTEVVGGAPQHEQIGK [648], STEEFPPLQPILQQK [654], |
| | TCLEER [678], THQAFMRK [698], TINFAR [701], TYLAVGSMK |
| | [740] |

c - Chronic lymphocytic leukaemia

| | |
|---|---|
| 95173 | DNQWLGVTLSR [147], ELNILHEMK [205], |
| | SQHTTEVVGGAPQHEQIGK [648] |
| 168205 | EVPGYIVLFYNMSLDVNR [235], ILELEEK [376], |
| | SQHTTEVVGGAPQHEQIGK [648], STEEFPPLQPILQQK [654] |

d - Hepatocellular carcmoma

| | |
|---|---|
| 66260 | IEGLQISK [361], VIELNK [768] |

OGTA159

TABLE 28

| | | |
|---|---|---|
| MW (Da) | Subfractionation | Tryptics identified [SEQ ID No] |

a - Breast cancer

| | |
|---|---|
| Unknown | ESPAQAPA [226], VAQPGPLEPEEPR [745] |

b - Chronic lymphocytic leukaemia

| | |
|---|---|
| 24836 | LEEEQK [450] |

c - Colorectal cancer

| | |
|---|---|
| 42137 | AASAGQEPLHNEELAGAGR [53], EEQAQR [180], |
| | VAQPGPLEPEEPR [745] |
| 42590 | AASAGQEPLHNEELAGAGR [53], VAQPGPLEPEEPR [745] |

d - Hepatocellular carcinoma

| | |
|---|---|
| 44183 | VAQPGPLEPEEPR [745] |

e - Melanoma

| | |
|---|---|
| 21554 | IQDLLAEGTITGVIDDR [387], VVLLEDLASQVGLR [811] |
| 21899 | RDLGSR [598], VVLLEDLASQVGLR [811] |

f - Pancreatic cancer

| | |
|---|---|
| 33531 | AASAGQEPLHNEELAGAGR [53], EHEEYLK [193], |
| | VAQPGPLEPEEPR [745] |
| 33932 | AASAGQEPLHNEELAGAGR [53], VAQPGPLEPEEPR [745] |

OGTA168

TABLE 29

| | | |
|---|---|---|
| MW (Da) | Subfractionation | Tryptics identified [SEQ ID No] |

a - Glioblastoma

| | | |
|---|---|---|
| 82431 | MonoQ/Digitonin | AISTNSELSTFR [74], ESHWIGLTDSER [225], |
| | | GSPGKPGPQGSSGDPGPPGPPGK [317], LQASGDALVDR [485], |
| | | NDFQNLQQVFLQAK [534], NLITNLQR [543] |
| 99352 | MonoQ/Digitonin | GSPGKPGPQGSSGDPGPPGPPGK [317], GSQGPPGPTGNK [319], |
| | | KLGDQTGK [423], LDTEVANLSVIMEEMK [448], LGDQTGKK |
| | | [461], LQASGDALVDR [485], NDFQNLQQVFLQAK [534], |
| | | SVDDTSQAIQR [661] |

b - Melanoma

| | |
|---|---|
| 33451 | GEPGPPGPAGER [292], LTAVESDLK [495], SVDDTSQAIQR |
| | [661] |

OGTA169

TABLE 30

| | | |
|---|---|---|
| MW (Da) | Subfractionation | Tryptics identified [SEQ ID No] |

a - Breast cancer

| | | |
|---|---|---|
| 100968 | PNGase F | FGAALTVLGDVNGDK [252] |
| | Deglycosylation | |

b - Chronic lymphocytic leukaemia

| | | |
|---|---|---|
| | Triton X114, | AVIFTQVSR [96], DLQSSVTLDLALDPGR [138], |
| | Detergent Soluble | ELNDIASKPSQEHIFK [204], ESHVAMHR [224], FQTHFTFEEFR |
| | | [269], FQTHFTFEEFRR [270], GAVYLFHGVLGPSISPSHSQR |
| | | [287], GVQSLVLGAPR [327], IAGSQLSSR [350], |
| | | IAPPASDFLAHIQK [352], INHLIFR [383], LFHASYGAR [458], |
| | | LTDVVIGAPGEEENR [496], NFATMMNFVR [535], |
| | | NLRPMLAADAQR [545], QEQDIVFLIDGSGSISSR [567], |
| | | SLHLTCDSAPVGSQGTWSTSCR [641], VEDFDALK [752], |
| | | YAVYTVVSSHEQFTK [841] |
| 108778 | | AVIFTQVSR [96], DVIPMADAAGIIR [161], ESHVAMHR [224], |
| | | FQTHFTFEEFR [269], GVQSLVLGAPR [327], INHLIFR [383], |
| | | LFHASYGAR [458], LTDVVIGAPGEEENR [496], |
| | | NLRPMLAADAQR [545], QEQDIVFLIDGSGSISSR [567], |
| | | YAVYTVVSSHEQFTK [841] |
| 111501 | | AVIFTQVSR [96], DVIPMADAAGIIR [161], |
| | | ELNDIASKPSQEHIFK [204], ESHVAMHR [224], |
| | | FGAALTVLGDVNGDK [252], FQTHFTFEEFR [269], |
| | | FQTHFTFEEFRR [270], GAVYLFHGVLGPSISPSHSQR [287], |
| | | GVQSLVLGAPR [327], IAGSQLSSR [350], INHLIFR [383], |
| | | LFHASYGAR [458], LTDVVIGAPGEEENR [496], |
| | | NFATMMNFVR [535], NLRPMLAADAQR [545], |
| | | NMYLTGLCFLLGPTQLTQR [548], QEQDIVFLIDGSGSISSR |
| | | [567], YAIGVGLAFQNR [839], YAVYTVVSSHEQFTK [841], |
| | | YQVNNLGQR [868] |
| 114387 | | AVIFTQVSR [96], DVIPMADAAGIIR [161], |
| | | EMMEEANGQIAPENGTQTPSPPSEK [210], ESHVAMHR [224], |
| | | FQTHFTFEEFR [269], FQTHFTFEEFRR [270], |
| | | GGAQITFLATFDVSPK [296], IAGSQLSSR [350], INHLIFR [383], |
| | | KEGDSLDYK [409], LFHASYGAR [458], LTDVVIGAPGEEENR |
| | | [496], NFATMMNFVR [535], NLRPMLAADAQR [545], |
| | | VEDFDALK [752], YAIGVGLAFQNR [839], |
| | | YAVYTVVSSHEQFTK [841], YQVNNLGQR [868] |
| 117454 | | DVIPMADAAGIIR [161], FQTHFTFEEFR [269], GGQVSVCPLPR |
| | | [299], IAGSQLSSR [350], INHLIFR [383], KEGDSLDYK [409], |
| | | LFHASYGAR [458], LTDVVIGAPGEEENR [496], |
| | | NMYLTGLCFLLGPTQLTQR [548], QEQDIVFLIDGSGSISSR |
| | | [567], YQVNNLGQR [868] |
| 136634 | | AVIFTQVSR [96], CDVPSFSVQEELDFTLK [100], |
| | | FQTHFTFEEFR [269], FQTHFTFEEFRR [270], GVQSLVLGAPR |
| | | [327], INHLIFR [383], KEGDSLDYK [409], |
| | | LTDVVIGAPGEEENR [496], NFATMMNFVR [535], |
| | | NLRPMLAADAQR [545], NPVLDCSIAGCLR [552], |
| | | SLHLTCDSAPVGSQGTWSTSCR [641], YQHTGK [867] |
| 139470 | | AVIFTQVSR [96], DSYLGYSTELALWK [158], FQTHFTFEEFR |
| | | [269], GVQSLVLGAPR [327], INHLIFR [383], KEGDSLDYK |
| | | [409], LTDVVIGAPGEEENR [496], NFATMMNFVR [535], |
| | | NLRPMLAADAQR [545], QEQDIVFLIDGSGSISSR [567] |
| 142364 | | GGQVSVCPLPR [299] |
| 152450 | | AVIFTQVSR [96], AVISQFQRPSTQFSLMQFSNK [97], DIQNQLK |
| | | [130], DVIPMADAAGIIR [161], ELNDIASKPSQEHIFK [204], |
| | | ESHVAMHR [224], FQTHFTFEEFR [269], GNLSFGWVR [303], |
| | | GVQSLVLGAPR [327], IAPPASDFLAHIQK [352], INHLIFR [383], |
| | | KEGDSLDYK [409], LFHASYGAR [458], LFHASYGARR [459], |
| | | LTDVVIGAPGEEENR [496], NLRPMLAADAQR [545], |
| | | QEQDIVFLIDGSGSISSR [567], YAVYTVVSSHEQFTK [841], |
| | | YQVNNLGQR [868] |
| 159984 | | AVIFTQVSR [96], DIQNQLK [130], DVIPMADAAGIIR [161], |
| | | ESHVAMHR [224], FQTHFTFEEFR [269], |
| | | GAVYLFHGVLGPSISPSHSQR [287], GVQSLVLGAPR [327], |
| | | INHLIFR [383], KEGDSLDYK [409], LFHASYGAR [458], |
| | | LTDVVIGAPGEEENR [496], NLRPMLAADAQR [545], |
| | | NMYLTGLCFLLGPTQLTQR [548], QEQDIVFLIDGSGSISSR |
| | | [567], YAVYTVVSSHEQFTK [841], YQVNNLGQR [868] |

OGTA174

TABLE 31

| | | |
|---|---|---|
| MW (Da) | Subfractionation | Tryptics identified [SEQ ID No] |

a - Acute T-cell leukaemia

CQGQLEVYLK [107], HLPETEAGR [337], LSQCHELWER [490],
LSWYDPDFQAR [493], NSYCKK [554], QGAQWAALCDSSSAR
[573], SHAENPTASHVDNEYSQPPR [634],
TTPPTTRPPPTTTPEPTAPPR [734], VLALLCSGFQPK [771],
WEEVCR [826]
HLPETEAGR [337], NSYCKK [554], QGAQWAALCDSSSAR
[573], VLDAGDPTSR [775]
VLDAGDPTSR [775]

b - Chronic lymphocytic leukaemia

| | |
|---|---|
| 52248 | HLPETEAGR [337], LSWYDPDFQAR [493], |
| | TQDLENFLCNNLQCGSFLK [723] |
| 53229 | LSWYDPDFQAR [493] |
| 70807 | AQDPGEPR [81], HLPETEAGR [337], IQNSSCTSLEHCFRK |
| | [390], LSWYDPDFQAR [493], SHAENPTASHVDNEYSQPPR [634] |
| 72277 | EHQPLPIQWK [194], HLPETEAGR [337], LSQCHELWER [490], |
| | LSWYDPDFQAR [493], SHAENPTASHVDNEYSQPPR [634], |
| | VLDAGDPTSR [775] |
| 73777 | HLPETEAGR [337], LSWYDPDFQAR [493], |
| | SHAENPTASHVDNEYSQPPR [634], VLDAGDPTSR [775] |

OGTA176

TABLE 32

| | | |
|---|---|---|
| MW (Da) | Subfractionation | Tryptics identified [SEQ ID No] |

a - B-cell non-Hodgkin's lymphoma

| | |
|---|---|
| 33825 | ELAQSQEALQVEQR [202], ETLQSEEQQR [231], |
| | ITQLGQSAEDLQGSR [396], ITQLGQSAEDLQGSRR [397], |
| | YLQVSQQLQQTNR [862] |

b - Chronic lymphocytic leukaemia

| | |
|---|---|
| 38233 | ELAQSQEALQVEQR [202], ETLQSEEQQR [231], |
| | ETLQSEEQQRR [232], ITQLGQSAEDLQGSR [396], |
| | SEQPTASWR [630], SSLPYICEMTAFR [650], |
| | YLQVSQQLQQTNR [862] |
| 39836 | ELAQSQEALQVEQR [202], ITQLGQSAEDLQGSR [396] |
| 40663 | ELAQSQEALQVEQR [202], ITQLGQSAEDLQGSR [396] |

OGTA177

TABLE 33
Chronic lymphocytic leukaemia

| MW (Da) | Subfractionation | Tryptics identified [SEQ ID No] |
|---|---|---|
| 81514 | | FLNLTSEK [259], MESEELADR [514], |
| | | SLSNYPFDFQGAR [643] |
| 81757 | | SLSNYPFDFQGAR [643] |
| 83454 | | DIQVASNEILR [131], FLNLTSEK [259], |
| | | SLSNYPFDFQGAR [643], SQHQETPVYLGATAGMR [647], |
| | | YGIVLDAGSSHTSLYIYK [853], VTEMMK [801] |
| 83957 | | DIQVASNEILR [131], SLSNYPFDFQGAR [643], |
| | | SQHQETPVYLGATAGMR [647], VTEMMKK [802] |
| 85186 | | SLSNYPFDFQGAR [643] |
| 86534 | | SQHQETPVYLGATAGMR [647] |
| 86954 | | DIQVASNEILR [131], SLSNYPFDFQGAR [643] |

OGTA197

TABLE 34

| | | |
|---|---|---|
| MW (Da) | Subfractionation | Tryptics identified [SEQ ID No] |

a - Colorectal cancer

| | |
|---|---|
| 93409 | QSPEDVYFSK [587], TYVDPHTYEDPNQAVLK [742], |
| | WTPPQDSGGR [837], YKPMMIITEYMENGALDK [857] |
| 95846 | MQQYTEHFMAAGYTAIEK [523], NGVSGLVTSR [538], |
| | VIGAGEFGEVYK [769], WTPPQDSGGR [837], |
| | YKPMMIITEYMENGALDK [857] |
| 101142 | QSPEDVYFSK [587], TSVTVSDLEPHMNYTFTVEAR [733], |
| | YKPMMIITEYMENGALDK [857] |

b - Hepatocellular carcmoma

| | |
|---|---|
| 75736 | AINDGFR [73], LPSTSGSEGVPFR [481], TLADFDPR [702], |
| | YLANMNYVHR [860], YSEPPHGLTR [871] |
| 82606 | AINDGFR [73], EVVLLDFAAAGGELGWLTHPYGK [239], |
| | IDTIAPDEITVSSDFEAR [359], LPSTSGSEGVPFR [481], RKNQR |
| | [605], TYVDPHTYEDPNQAVLK [742], |
| | VDFLGEAGIMGQFSHHNIIR [747], VVQMTNDDIK [813], |
| | YLANMNYVHR [860], YSEPPHGLTR [871] |
| 84442 | AINDGFR [73], FTTEIHPSCVTR [278], IDTIAPDEITVSSDFEAR |
| | [359], LPSTSGSEGVPFR [481], MQQYTEHFMAAGYTAIEK |
| | [523], TYVDPHTYEDPNQAVLK [742], VVQMTNDDIKR [814], |
| | YKPMMIITEYMENGALDK [857], YLANMNYVHR [860], |
| | YSEPPHGLTR [871] |
| 86381 | AINDGFR [73], QSPEDVYFSK [587] |
| 88432 | AINDGFR [73], LPGHQKR [478], QSPEDVYFSK [587], |
| | VIGAGEFGEVYK [769], VSDFGLSR [796], VVQMTNDDIK |
| | [813], VVQMTNDDIKR [814], WTAPEAISYR [834], |
| | WTAPEAISYRK [835], YKPMMIITEYMENGALDK [857], |
| | YSEPPHGLTR [871] |
| 92956 | KGDSNSYNVR [417], QSPEDVYFSK [587], RKNQR [605], |
| | VIGAGEFGEVYK [769], VVQMTNDDIK [813], WTAPEAISYR |
| | [834], YLANMNYVHR [860], YSEPPHGLTR [871] |
| 116695 | LPSTSGSEGVPFR [481], MELQAAR [512], |
| | MQQYTEHFMAAGYTAIEK [523], TLADFDPR [702], VYYKK |
| | [822], YKPMMIITEYMENGALDK [857] |

c - Lung cancer

| | |
|---|---|
| | DQVNTVGIPI [154], EVPVAIK [238], FTTEIHPSCVTR [278], |
| | IDTIAPDEITVSSDFEAR [359], KKGDSNSYNVR [422], |
| | VIGAGEFGEVYK [769], VLEDDPEATYTTSGGK [777], |
| | VVQMTNDDIK [813], VVQMTNDDIKR [814], |
| | YKPMMIITEYMENGALDK [857] |
| | KEVPVAIK [414], LPSTSGSEGVPFR [481], |
| | TSVTVSDLEPHMNYTFTVEAR [733], VLEDDPEATYTTSGGK |
| | [777], WTPPQDSGGR [837], YEVTYR [845], YLANMNYVHR |
| | [860], YSEPPHGLTR [871] |
| 113648 | DGEFSVLQLVGMLR [121], LPSTSGSEGVPFR [481], |
| | MQQYTEHFMAAGYTAIEK [523], QSPEDVYFSK [587], RKNQR |
| | [605], VDFLGEAGIMGQFSHHNIIR [747], VIGAGEFGEVYK |
| | [769], VVQMTNDDIK [813], WTAPEAISYRK [835], |
| | YKPMMIITEYMENGALDK [857], YLANMNYVHR [860] |
| 116436 | AINDGFR [73], FADIVSILDK [242], FTTEIHPSCVTR [278], |
| | GDSNSYNVRR [288], LPSTSGSEGVPFR [481], |
| | MQQYTEHFMAAGYTAIEK [523], NGVSGLVTSR [538], |
| | TASVSINQTEPPK [673], TLADFDPR [702], TVSEWLESIK [738], |
| | VIGAGEFGEVYK [769], WTPPQDSGGR [837], YEVTYR [845], |
| | YKPMMIITEYMENGALDK [857], YLANMNYVHR [860] |
| 122412 | LPSTSGSEGVPFR [481], VYYKK [822], YLANMNYVHR [860], |
| | QSPEDVYFSK [587] |

d - Melanoma

| | |
|---|---|
| 78319 | APQDPASMPCTRPPSAPHYLTAVGMGAK [80], |
| | DGEFSVLQLVGMLR [121], EVPVAIK [238], FADIVSILDK |
| | [242], MELQAAR [512], STTSLSVSWSIPPPQQSR [660], |
| | SVGPLTR [662], TASVSINQTEPPK [673], |
| | TSVTVSDLEPHMNYTFTVEAR [733], VIGAGEFGEVYK [769], |
| | YKPMMIITEYMENGALDK [857], YLANMNYVHR [860] |

e - Osteosarcoma

| | |
|---|---|
| 30076 | LPSTSGSEGVPFR [481], VVQMTNDDIK [813], VVQMTNDDIKR |
| | [814], YEVTYRK [846], YKPMMIITEYMENGALDK [857], |
| | YLANMNYVHR [860] |
| 55008 | AINDGFR [73], APQDPASMPCTRPPSAPHYLTAVGMGAK [80], |
| | DGEFSVLQLVGMLR [121], IDTIAPDEITVSSDFEAR [359], |
| | RKNQR [605], STTSLSVSWSIPPPQQSR [660], SVGPLTR [662], |
| | TASVSINQTEPPK [673], TNWVYR [715], |
| | TSVTVSDLEPHMNYTFTVEAR [733], TYVDPHTYEDPNQAVLK |
| | [742], VLEDDPEATYTTSGGK [777], WTPPQDSGGR [837], |
| | YKPMMIITEYMENGALDK [857] |
| 67385 | AINDGFR [73], IDTIAPDEITVSSDFEAR [359], KGDSNSYNVR |
| | [417], LPTPMDCPSAIYQLMMQCWQQER [483], |
| | MQQYTEHFMAAGYTAIEK [523], NGVSGLVTSR [538], |
| | STTSLSVSWSIPPPQQSR [660], SVGPLTR [662], |
| | TASVSINQTEPPK [673], TLADFDPR [702], TNWVYR [715], |
| | TSVTVSDLEPHMNYTFTVEAR [733], TYVDPHTYEDPNQAVLK |
| | [742], VDFLGEAGIMGQFSHHNIIR [747], WTPPQDSGGR [837], |
| | YSEPPHGLTR [871] |
| 93496 | ACFALLWGCALAAAAAAQGK [55], AINDGFR [73], |
| | FTTEIHPSCVTR [278], IDTIAPDEITVSSDFEAR [359], RKNQR |
| | [605], STTSLSVSWSIPPPQQSR [660], SVGPLTR [662], |
| | TASVSINQTEPPK [673], TNWVYR [715], |
| | TYVDPHTYEDPNQAVLK [742], VIGAGEFGEVYK [769], |
| | VVQMTNDDIKR [814], YEVTYRK [846], YLANMNYVHR [860], |
| | YSEPPHGLTR [871] |

f - Ovarian cancer

| | |
|---|---|
| 63557 | AINDGFR [73], FTTEIHPSCVTR [278], LPSTSGSEGVPFR [481], |
| | STTSLSVSWSIPPPQQSR [660], TASVSINQTEPPK [673], |
| | TNWVYR [715], VVQMTNDDIKR [814], WTPPQDSGGR [837], |
| | YEVTYR [845], YLANMNYVHR [860], YSEPPHGLTR [871] |
| 67618 | ACFALLWGCALAAAAAAQGK [55], QSPEDVYFSK [587], |
| | RKNQR [605], SVGPLTR [662], TASVSINQTEPPK [673], |
| | TLADFDPR [702], TSVTVSDLEPHMNYTFTVEAR [733], |
| | VDFLGEAGIMGQFSHHNIIR [747], WTPPQDSGGR [837], |
| | YKPMMIITEYMENGALDK [857] |
| 69811 | FADIVSILDK [242], KGDSNSYNVRR [418], RKNQR [605], |
| | TASVSINQTEPPK [673], TYVDPHTYEDPNQAVLK [742], |
| | VSDFGLSR [796] |
| 79417 | ACFALLWGCALAAAAAAQGK [55], DGEFSVLQLVGMLR |
| | [121], IDTIAPDEITVSSDFEAR [359], SVGPLTR [662], |
| | TSVTVSDLEPHMNYTFTVEAR [733], TYVDPHTYEDPNQAVLK |
| | [742], VIGAGEFGEVYK [769], YKPMMIITEYMENGALDK |
| | [857], YLANMNYVHR [860], YSEPPHGLTR [871] |

g - Pancreatic cancer

| | |
|---|---|
| 53057 | AINDGFR [73], IDTIAPDEITVSSDFEAR [359], LEGVISK [454], |
| | LNVEER [474], LPSTSGSEGVPFR [481], SVGPLTR [662], |
| | VIGAGEFGEVYK [769], VVQMTNDDIK [813], YEVTYRKK |
| | [847] |
| 53737 | IDTIAPDEITVSSDFEAR [359], LPSTSGSEGVPFR [481], |
| | SEQLKPLK [629], SVGPLTRK [663], YLANMNYVHR [860] |
| 93964 | LPSTSGSEGVPFR [481], TVSEWLESIK [738], WTPPQDSGGR |
| | [837], YSEPPHGLTR [871] |
| 102286 | VIGAGEFGEVYK [769], VVQMTNDDIKR [814], WTPPQDSGGR |
| | [837], YSEPPHGLTR [871] |
| 107693 | KGDSNSYNVR [417], QSPEDVYFSK [587], TNWVYR [715], |
| | TYVDPHTYEDPNQAVLK [742], VIGAGEFGEVYK [769], |
| | VVQMTNDDIKR [814], WTPPQDSGGR [837], YLANMNYVHR |
| | [860], YSEPPHGLTR [871] |
| 110489 | TASVSINQTEPPK [673], TLADFDPR [702], |
| | TYVDPHTYEDPNQAVLK [742], VIGAGEFGEVYK [769], |
| | WTPPQDSGGR [837] |
| 113324 | FADIVSILDK [242], IDTIAPDEITVSSDFEAR [359], |
| | KGDSNSYNVR [417], SVGPLTR [662], TLADFDPR [702], |
| | TYVDPHTYEDPNQAVLK [742], VYYKK [822], WTPPQDSGGR |
| | [837], YEVTYR [845], YEVTYRK [846], YLANMNYVHR [860], |
| | YSEPPHGLTR [871] |
| 116302 | IDTIAPDEITVSSDFEAR [359], TYVDPHTYEDPNQAVLK [742], |
| | VVQMTNDDIK [813], WTPPQDSGGR [837], YEVTYR [845], |
| | YLANMNYVHR [860], YSEPPHGLTR [871] |
| 116866 | ACFALLWGCALAAAAAAQGK [55], AINDGFR [73], |
| | IDTIAPDEITVSSDFEAR [359], LPSTSGSEGVPFR [481], |
| | STTSLSVSWSIPPPQQSR [660], TYVDPHTYEDPNQAVLK [742], |
| | VIGAGEFGEVYK [769], VLEDDPEATYTTSGGK [777], |
| | WTAPEAISYR [834], WTPPQDSGGR [837], |
| | YKPMMIITEYMENGALDK [857], YLANMNYVHR [860], |
| | YSEPPHGLTR [871] |
| 122219 | ACFALLWGCALAAAAAAQGK [55], AINDGFR [73], |
| | IDTIAPDEITVSSDFEAR [359], KGDSNSYNVR [417], |
| | LPSTSGSEGVPFR [481], MQQYTEHFMAAGYTAIEK [523], |
| | QSPEDVYFSK [587], STTSLSVSWSIPPPQQSR [660], TNWVYR |
| | [715], VIGAGEFGEVYK [769], WTAPEAISYR [834], |
| | WTPPQDSGGR [837], YKPMMIITEYMENGALDK [857], |
| | YSEPPHGLTR [871] |
| 180074 | KGDSNSYNVR [417], KGDSNSYNVRR [418], LPSTSGSEGVPFR |
| | [481], MELQAAR [512], QSPEDVYFSK [587], TNWVYR [715], |
| | WTAPEAISYR [834], WTPPQDSGGR [837], YEVTYR [845], |
| | YSEPPHGLTR [871] |
| 184373 | AINDGFR [73], LPSTSGSEGVPFR [481], QSPEDVYFSK [587], |
| | TNWVYR [715], WTAPEAISYR [834], WTPPQDSGGR [837], |
| | YEVTYR [845], YLANMNYVHR [860], YSEPPHGLTR [871] |
| 188775 | LPSTSGSEGVPFR [481], TNWVYR [715], VIGAGEFGEVYK |
| | [769], VVQMTNDDIK [813], WTAPEAISYR [834], |
| | WTPPQDSGGR [837], YLANMNYVHR [860], YSEPPHGLTR |
| | [871] |

h - Prostate cancer

LPSTSGSEGVPFR [481], MELQAAR [512], YEVTYR [845]
LPSTSGSEGVPFR [481], NGVSGLVTSR [538], VVQMTNDDIK
[813], WTPPQDSGGR [837], YSEPPHGLTR [871]

i - Renal cell cancer

| | |
|---|---|
| 88378 | AINDGFR [73], KGDSNSYNVRR [418], LPSTSGSEGVPFR [481], |
| | TNWVYR [715], WTAPEAISYR [834], WTPPQDSGGR [837], |
| | YSEPPHGLTR [871] |
| 90442 | AINDGFR [73], LPSTSGSEGVPFR [481], SVGPLTR [662], |
| | TNWVYR [715], TYVDPHTYEDPNQAVLK [742], |
| | VIGAGEFGEVYK [769], VVQMTNDDIKR [814], WTAPEAISYR |
| | [834], WTPPQDSGGR [837], YEVTYR [845], YLANMNYVHR |
| | [860], YSEPPHGLTR [871] |
| 117578 | AINDGFR [73], TNWVYR [715], VIGAGEFGEVYK [769], |
| | VVQMTNDDIK [813], VVQMTNDDIKR [814], WTAPEAISYR |
| | [834], YLANMNYVHR [860], YSEPPHGLTR [871] |
| 122597 | ACFALLWGCALAAAAAAQGK [55], TYVDPHTYEDPNQAVLK |
| | [742], VIGAGEFGEVYK [769], VVQMTNDDIKR [814], |
| | WTAPEAISYR [834], YEVTYRK [846], YLANMNYVHR [860], |
| | YSEPPHGLTR [871] |

OGTA202

TABLE 35
Colorectal cancer

| | | Tryptics identified |
|---|---|---|
| MW (Da) | Subfractionation | [SEQ ID No] |
| 51531 | Nucleotide Binding | FTQDTFR [277] |

OGTA203

TABLE 36

| | | |
|---|---|---|
| MW | | |
| (Da) | Subfractionation | Tryptics identified [SEQ ID No] |

a-Ovarian cancer

| | |
|---|---|
| 69811 | DNIVSYNDEGGGEEDTQAFDIGTLR [144], |
| | EDAQINTTIGSVTAQDPDAAR [168], RTPTAR [618], |
| | TALLNMDR [669], TPESSPPGTPIGR [716], |
| | TSGFPAKK [727], VEASNPYVEPR [751] |

b-Renal cell cancer

| | |
|---|---|
| 82732 | IVVEDVDEPPVFSK [402], KNGYNR [428], |
| | TSGFPAKK [727] |

OGTA206 a-Breast cancer

| | | |
|---|---|---|
| 27751 | Vesicles | VYDSLLALPQDLQAAR [817] |

b-Pancreatic cancer

| | |
|---|---|
| 26738 | VYDSLLALPQDLQAAR [817] |
| 27019 | VYDSLLALPQDLQAAR [817] |

c-Prostate cancer

| | |
|---|---|
| Digitonin | VYDSLLALPQDLQAAR [817] |

OGTA213

TABLE 38
Lung cancer

| MW | | Tryptics identified |
|---|---|---|
| (Da) | Subfractionation | [SEQ ID No] |
| 27644 | | IVFTPTICK [401] |

OGTA214

TABLE 39
Ovarian cancer

| MW | | Tryptics identified |
|---|---|---|
| (Da) | Subfractionation | [SEQ ID No] |
| 65600 | | QCLIKK [565], |
| | | SSSSNFLNYDLTLR [651] |
| 79331 | | ISVTVSETFDPEEK [394], |
| | | SSSSNFLNYDLTLR [651] |
| 89348 | | ISVTVSETFDPEEK [394], |
| | | SSSSNFLNYDLTLR [651] |

OGTA216

TABLE 40

| | | |
|---|---|---|
| MW | | |
| (Da) | Subfractionation | Tryptics identified [SEQ ID No] |

a-Breast cancer

| | |
|---|---|
| 196008 | AFYAPVHADDLR [69], EGAQYLMQAAGLGR [186], |
| | FQVDLVSENAGR [271], GPIVPLNVADQK [307], IDHDRR [356], |
| | KDWLQADMR [408], NVGLMICGHVHMGPR [555], |
| | SEIFNENFGPDFR [626] |
| 248063 | DEGPAAAGDGLGRPLGPTPSQSR [114], DVVVSVEYSK [163], |
| | EGAQYLMQAAGLGR [186], FQVDLVSENAGR [271], |
| | NVGLMICGHVHMGPRR [556], SEIFNENFGPDFR [626] |

b-Colorectal cancer

| | | |
|---|---|---|
| | Heparin Binding | DAVVTYTAESK [110], DVVVSVEYSK [163], |
| | | SEIFNENFGPDFR [626] |
| | Heparin Binding | KENIIAFEEIIEPYR [410], SEIFNENFGPDFR [626] |
| | Heparin Binding | KENIIAFEEIIEPYR [410], LNELLK [471], |
| | | SEIFNENFGPDFR [626] |
| | Heparin Binding | AFYAPVHADDLR [69], EHSSTANIIVMSLPVAR [195], |
| | | FQVDLVSENAGR [271], SEIFNENFGPDFR [626], |
| | | SPGWRPAFK [645] |
| | Heparin Binding | DEGPAAAGDGLGRPLGPTPSQSR [114], GFFGYK [294], |
| | | KENIIAFEEIIEPYR [410], SEIFNENFGPDFR [626] |
| | Heparin Binding | FQVDLVSENAGR [271], GGGAYYLISR [298], |
| | | KENIIAFEEIIEPYR [410], SEIFNENFGPDFR [626] |
| | Heparin Binding | AAAAAAAAAAAAAAAGAGAGAK [49], DLPPILLVR [136], |
| | | DNIYPAFQMFAK [145], EGAQYLMQAAGLGR [186], |
| | | EHSSTANIIVMSLPVAR [195], EPFEDGFANGEESTPTR [216], |
| | | FQVDLVSENAGR [271], IDFSDIMVLGDINTKPK [354], |
| | | IDHYR [357], KENIIAFEEIIEPYR [410], |
| | | SEIFNENFGPDFR [626] |
| | Heparin Binding | CMLNIWGVMLFIR [106], IDHDRR [356], ITDNELELYK [395], |
| | | SEIFNENFGPDFR [626] |
| | Heparin Binding | DLPPILLVR [136], NVGLMICGHVHMGPR [555], |
| | | SEIFNENFGPDFR [626] |
| | Heparin Binding | AMATLLSK [78], EGAQYLMQAAGLGR [186], GGGAYYLISR [298], |
| | | GPIVPLNVADQK [307], NVGLMICGHVHMGPRR [556], |
| | | SPGWRPAFK [645] |
| 121506 | | DEGPAAAGDGLGRPLGPTPSQSR [114], DLPPILLVR [136], |
| | | DNIYPAFQMFAK [145], DWLQADMR [164], |
| | | EGAQYLMQAAGLGR [186], EPFEDGFANGEESTPTR [216], |
| | | IDFSDIMVLGDINTKPKK [355], IDHYR [357], |
| | | KENIIAFEEIIEPYR [410], KSDLDTSKPLSEKPITHK [435], |
| | | LNELLK [471], NVGLMICGHVHMGPRR |
| | | SEIFNENFGPDFR [626], TFGHNTMDAVPR [688], |
| | | VELPGTAVPSVPEDAAPASR [754] |
| 125772 | | DLPPILLVR [136], DWLQADMR [164], EPFEDGFANGEESTPTR |
| | | [216], KDWLQADMR [408], SEIFNENFGPDFR [626], |
| | | SPGWRPAFK [645], TFGHNTMDAVPR [688], |
| | | VELPGTAVPSVPEDAAPASR [754] |
| 140688 | | DLPPILLVR [136], DWLQADMR [164], EGAQYLMQAAGLGR [186], |
| | | EPFEDGFANGEESTPTR [216], KDWLQADMR [408], |
| | | KSDLDTSKPLSEKPITHK [435], NVGLMICGHVHMGPR [555], |
| | | TFGHNTMDAVPR [688], VELPGTAVPSVPEDAAPASR [754] |
| 146522 | | DLPPILLVR [136], DWLQADMR [164], EGAQYLMQAAGLGR [186], |
| | | EPFEDGFANGEESTPTR [216], IDHDRR [356], |
| | | KDWLQADMR [408], KENIIAFEEIIEPYR [410], |
| | | KSDLDTSKPLSEKPITHK [435], SEIFNENFGPDFR [626], |
| | | TFGHNTMDAVPR [688], VELPGTAVPSVPEDAAPASR [754] |
| 167535 | | DWLQADMR [164], EPFEDGFANGEESTPTR [216], |
| | | GPIVPLNVADQK [307], IDHDRR [356], SEIFNENFGPDFR [626], |
| | | TFGHNTMDAVPR [688], VELPGTAVPSVPEDAAPASR [754] |
| 175447 | Nucleotide | NNEPLR [549], SEIFNENFGPDFR [626], TFGHNTMDAVPR [688], |
| | Binding | VELPGTAVPSVPEDAAPASR [754] |
| 190098 | Nucleotide | DAVVTYTAESK [110], EGAQYLMQAAGLGR [186], |
| | Binding | EMSIDQAK [213], LHEDDK [465], LNELLK [471], |
| | | RAMATLLSK [596], SEIFNENFGPDFR [626], |
| | | SPGWRPAFK [645], TFGHNTMDAVPR [688] |
| 218400 | Nucleotide | DEGPAAAGDGLGRPLGPTPSQSR [114], EGAQYLMQAAGLGR [186], |
| | Binding | KENIIAFEEIIEPYR [410], KSDLDTSKPLSEKPITHK [435], |
| | | SEIFNENFGPDFR [626] |
| 221484 | | DEGPAAAGDGLGRPLGPTPSQSR [114], EGAQYLMQAAGLGR [186], |
| | | EPFEDGFANGEESTPTR [216], IDHDRR [356], |
| | | KDWLQADMR [408], NVGLMICGHVHMGPRR [556], |
| | | SEIFNENFGPDFR [626], TFGHNTMDAVPR [688] |
| 255000 | | EGAQYLMQAAGLGR [186], EPFEDGFANGEESTPTR [216], |
| | | SEIFNENFGPDFR [626], TFGHNTMDAVPR [688], |
| | | VELPGTAVPSVPEDAAPASR [754] |
| 301246 | | DEGPAAAGDGLGRPLGPTPSQSR [114], EGAQYLMQAAGLGR [186], |
| | | EPFEDGFANGEESTPTR [216], IDHDRR [356], IDHYR [357], |
| | | SEIFNENFGPDFR [626], TFGHNTMDAVPR [688] |

c - Pancreatic cancer

| | |
|---|---|
| 148609 | AFYAPVHADDLR [69], DVVVSVEYSK [163], GGGAYYLISR [298], |
| | NNEPLR [549], SEIFNENFGPDFR [626], |
| | VELPGTAVPSVPEDAAPASR [754] |
| 154726 | DVVVSVEYSK [163], EPFEDGFANGEESTPTR [216], |
| | GPIVPLNVADQK [307], NVGLMICGHVHMGPR [555], |
| | SEIFNENFGPDFR [626] |

OGTA222

TABLE 41

| | | |
|---|---|---|
| MW | | |
| (Da) | Subfractionation | Tryptics identified [SEQ ID No] |

a-B-cell non-Hodgkin's lymphoma

| | |
|---|---|
| 45662 | AVGFGGDFDGVPR [95], TLEQMDVVHR [703] |
| 48028 | ALYQLGMR [77], AVGFGGDFDGVPR [95], |
| | GALADNLLR [283], MYPETFLYVTSSAGIR [530], |
| | NVPDDVLR [559], RTLEQMDVVHR [617], |
| | TLEQMDVVHR [703], YPDLIAELLR [864], |
| | VPEGLEDVSK [791] |
| 48530 | ALYQLGMR [77], AVGFGGDFDGVPR [95], |
| | GALADNLLR [283], MYPETFLYVTSSAGIR [530], |
| | TLEQMDVVHR [703], VPEGLEDVSK [791] |
| 49042 | ALYQLGMR [77], AVGFGGDFDGVPR [95], |
| | GALADNLLR [283], MYPETFLYVTSSAGIR [530], |
| | TLEQMDVVHR [703], VPEGLEDVSK [791] |
| 66777 | ALYQLGMR [77], AVGFGGDFDGVPR [95], |
| | GALADNLLR [283], TLEQMDVVHR [703], ATLQLSR [91] |
| 68315 | ALYQLGMR [77], AVGFGGDFDGVPR [95], |
| | GALADNLLR [283], TLEQMDVVHR [703] |
| 69923 | ALYQLGMR [77], ATLQLSR [91], AVGFGGDFDGVPR [95], |
| | GALADNLLR [283], TLEQMDVVHR [703] |
| 71607 | ALYQLGMR [77], AVGFGGDFDGVPR [95], |
| | GALADNLLR [283], TLEQMDVVHR [703], |
| | VPEGLEDVSK [791] |

b - Colorectal cancer

| | |
|---|---|
| 52940 | ALYQLGMR [77], AVGFGGDFDGVPR [95], |
| | GALADNLLR [283], TLEQMDVVHR [703] |
| 55204 | AVGFGGDFDGVPR [95] |
| 58558 | AVGFGGDFDGVPR [95], TLEQMDVVHR [703] |

OGTA236

TABLE 42
Pancreatic cancer

| MW | | Tryptics identified |
|---|---|---|
| (Da) | Subfractionation | [SEQ ID No] |
| 73411 | | SVLGVITIVNLR [664] |

OGTA237

TABLE 43

| | | |
|---|---|---|
| | | Tryptics |
| MW | | identified |
| (Da) | Subfractionation | [SEQ ID No] |

a-B-cell non-Hodgkin's lymphoma

| | |
|---|---|
| 47536 | IADFGLAR [349] |
| 48028 | IADFGLAR [349] |
| 48530 | IADFGLAR [349] |
| 49565 | IADFGLAR [349] |
| 50098 | IADFGLAR [349] |
| 51766 | IADFGLAR [349] |
| 52346 | IADFGLAR [349] |

b-Lymphoid leukaemia, unspecified

| | |
|---|---|
| 52041 | IADFGLAR [349] |
| 52618 | IADFGLAR [349] |
| 55054 | IADFGLAR [349] |

c-Prostate cancer

IADFGLAR [349]
IADFGLAR [349]

OGTA247

TABLE 44

| | | |
|---|---|---|
| MW | | |
| (Da) | Subfractionation | Tryptics identified [SEQ ID No] |

a-Hepatocellular carcinoma

| | |
|---|---|
| 102119 | DATQITQGPR [109], EDTQVDSEARPMK [170], GYNVTYWR [330], |
| | STIEKK [659], YDIEFEDK [842], YFIEDGR [850] |
| 114377 | DATQITQGPR [109], FFPYANGTLGIR [251], FHILFK [253], |
| | GYNVTYWR [330], LGTAMSHEIR [463], LSPYVHYTFR [489], |
| | STIEKK [659], VSWSPAEDHNAPIEK [798], |
| | VTFTCQASFDPSLQPSITWR [804], VTYQNHNK [808], |
| | WLRPSGPMPADR [828], WMDWNAPQVQYR [829], YDIEFEDK [842] |
| 116037 | DATQITQGPR [109], EPIDLR [217], FQGIYR [266], |
| | LDCQVQGRPQPEVTWR [445], LGTAMSHEIR [463], |
| | YFIEDGR [850] |
| 118513 | AQLLVVGSPGPVPR [82], ATNSMIDR [92], DATQITQGPR [109], |
| | EGPGEAIVR [189], FFPYANGTLGIR [251], LGTAMSHEIR [463], |
| | LSPYVHYTFR [489], VTYQNHNK [808], WLRPSGPMPADR [828], |
| | YFIEDGR [850] |
| 121636 | DATQITQGPR [109], EPIDLR [217], FHILFK [253], |
| | FQGIYR [266], YDIEFEDK [842], YFIEDGR [850] |
| 123033 | AQLLVVGSPGPVPR [82], CEASGKPEVQFR [101], |
| | DATQITQGPR [109], EGPGEAIVR [189], FFPYANGTLGIR [251], |
| | LSPYVHYTFR [489], VGEEDDGEYR [762], VQWRPQGTR [795], |
| | YFIEDGR [850] |
| 128000 | AQLLVVGSPGPVPR [82], DATQITQGPR [109], LGTAMSHEIR [463], |
| | LSPYVHYTFR [489], YFIEDGR [850] |
| 200565 | CEASGKPEVQFR [101], EPIDLR [217], INGIPVEELAK [382], |
| | VSWSPAEDHNAPIEK [798], WLRPSGPMPADR [828] |
| 209214 | ATNSMIDR [92], DATQITQGPR [109], EGPGEAIVR [189], |
| | EPIDLR [217], GALILSNVQPSDTMVTQCEAR [284], |
| | HQMAVK [342] |
| 218613 | AQLLVVGSPGPVPR [82], CEASGKPEVQFR [101], |
| | DLQELGDSDK [137], FFPYANGTLGIR [251], LGTAMSHEIR [463], |
| | WLRPSGPMPADR [828] |

b-Melanoma

| | |
|---|---|
| 146479 | AQLLVVGSPGPVPR [82], CEASGKPEVQFR [101], |
| | CLAENSLGSAR [105], DATQITQGPR [109], DGVHFKPK [126], |
| | EGPGEAIVR [189], EPIDLR [217], FQGIYR [266], GQLSFNLR |
| | [312], INGIPVEELAK [382], LGTAMSHEIR [463], LSPYVHYTFR |
| | [489], LVLSDLHLLTQSQVR [502], VGEEDDGEYR [762], |
| | VSWSPAEDHNAPIEK [798], WLRPSGPMPADR [828], |
| | WMDWNAPQVQYR [829], WRPVDLAQVK [831], YFIEDGR [850] |
| 152349 | AQLLVVGSPGPVPR [82], CLAENSLGSAR [105], DATQITQGPR |
| | [109], DGVHFKPK [126], EGPGEAIVR [189], EPIDLR [217], |
| | FQGIYR [266], LGTAMSHEIR [463], LSPYVHYTFR [489], |
| | LVLSDLHLLTQSQVR [502], WLRPSGPMPADR [828], |
| | WMDWNAPQVQYR [829], WRPVDLAQVK [831], YFIEDGR [850] |
| 163936 | AFGAPVPSVQWLDEDGTTVLQDER [65], AQLLVVGSPGPVPR [82], |
| | CEASGKPEVQFR [101], FQGIYR [266], GQLSNFLR [312], |
| | LGTAMSHEIR [463], LVLSDLHLLTQSQVR [502], |
| | VSWSPAEDHNAPIEK [798], WLRPSGPMPADR [828], |
| | WMDWNAPQVQYR [829], WRPVDLAQVK [831], YFIEDGR [850] |
| 172991 | AFGAPVPSVQWLDEDGTTVLQDER [65], AQLLVVGSPGPVPR [82], |
| | CEASGKPEVQFR [101], CLAENSLGSAR [105], |
| | DHVVVPANTTSVILSGLRPYSSYHLEVQAFNGR [128], |
| | EELGVTVYQSPHSGSFTITGNNSNFAQR [178], EGPGEAIVR [189], |
| | EPIDLR [217], FHILFK [253], FQGIYR [266], |
| | GQLSFNLR [312], LVLSDLHLLTQSQVR [502], VGEEDDGEYR [762], |
| | VQWRPQGTR [795], VSWSPAEDHNAPIEK [798], |
| | WLRPSGPMPADR [828], WMDWNAPQVQYR [829], |
| | WRPVDLAQVK [831], YFIEDGR [850] |
| 193221 | AFGAPVPSVQWLDEDGTTVLQDER [65], AQLLVVGSPGPVPR [82], |
| | FFPYANGTLGIR [251], FHILFK [253], FQGIYR [266], |
| | GALILSNVQPSDTMVTQCEAR [284], GEGNETTNMVITWKPLR [289], |
| | GQLSFNLR [312], INGIPVEELAK [382], LGTAMSHEIR [463], |
| | LVLSDLHLLTQSQVR [502], WLRPSGPMPADR [828], |
| | WMDWNAPQVQYR [829], WRPVDLAQVK [831] |

OGTA248

TABLE 45
Renal cell cancer

| MW | | Tryptics identified |
|---|---|---|
| (Da) | Subfractionation | [SEQ ID No] |
| 34832 | | TNQEVLDFVVGGGR [712] |

OGTA249

TABLE 46
Ovarian cancer

| MW | | Tryptics identified |
|---|---|---|
| (Da) | Subfractionation | [SEQ ID No] |
| | | VTLPAGPDLLR [805] |
| | | MSAGGASVPPPPNPAVSFPPPR [524] |

OGTA257

TABLE 47
Pancreatic cancer

| MW | | Tryptics identified |
|---|---|---|
| (Da) | Subfractionation | [SEQ ID No] |
| 176135 | | LLQEAYMTPK [468] |

OGTA271

TABLE 48

| | | |
|---|---|---|
| MW | | |
| (Da) | Subfractionation | Tryptics identified [SEQ ID No] |

a-Breast cancer

| | | |
|---|---|---|
| 24841 | Vesicles | AHTDVGPGPESSPVLVR [72], AYIATQGPLAESTEDFWR [98], |
| | | DHPPIPITDLADNIER [127], DSLLAHSSDPVEMRR [157], |
| | | GVEGSDYINASFLDGYR [326], LGQVPPGESVTAMELEFK [462], |
| | | LIADLQPNTEYSFVLMNR [467], MLWEHNSTIIVMLTK [519], |
| | | QFQFTDWPEQGVPK [572], |
| | | QNAYIATQGPLPETMGDFWR [581], SDMGVGVFTPTIEAR [621], |
| | | YEGVVDMFQTVK [844], YQYFVVDPMAEYNMPQYILR [869] |
| 24993 | Vesicles | AALEYLGSFDHYAT [51], AYIATQGPLAESTEDFWR [98], |
| | | DHPPIPITDLADNIER [127], DINSQQELQNITTDTR [129], |
| | | DSLLAHSSDPVEMRR [157], FDLSMPHVQDPSLVR [246], |
| | | GVEGSDYINASFLDGYR [326], HVVDGISR [347], |
| | | ITWMKK [399], KQNAYIATQGPLPETMGDFWR [43], |
| | | LGQVPPGESVTAMELEFK [462], LIADLQPNTEYSFVLMNR [467], |
| | | LSVLEEEQLPPGFPSIDMGPQLK [492], |
| | | MLSASTMLVQWEPPEEPNGLVR [518], MLWEHNSTIIVMLTK [519], |
| | | NGVITQYSVAYEAVDGEDR [537], QFQFTDWPEQGVPK [572], |
| | | TGEGFIDFIGQVHK [693], |
| | | VPEDQTGLSGGVASFVCQATGEPKPR [789], |
| | | YQYFVVDPMAEYNMPQYILR [869] |
| 55008 | | MVWEQR [529], YQYFVVDPMAEYNMPQYILR [869] |
| 55921 | | AALEYLGSFDHYAT [51], AYIATQGPLAESTEDFWR [98], |
| | | DFLPVDPATSNGR [117], DHPPIPITDLADNIER [127], |
| | | EHSSWDLVGLEK [196], GVEGSDYINASFLDGYR [326], |
| | | HVVDGISR [347], ILYNGQSVEVDGHSMRK [379], |
| | | KLIADLQPNTEYSFVLMNR [424], LENGEPR [455], |
| | | LGQVPPGESVTAMELEFK [462], LNYQTPGMR [475], |
| | | MLWEHNSTIIVMLTK [519], MVWEQR [529], |
| | | NGVITQYSVAYEAVDGEDR [537], QFQFTDWPEQGVPK [572], |
| | | RLNYQTPGMR [606], TGEGFIDFIGQVHK [693], |
| | | YEGVVDMFQTVK [844] |
| 56324 | | DFLPVDPATSNGR [117], DHPPIPITDLADNIER [127], |
| | | ILYNGQSVEVDGHSMR [378], LNYQTPGMR [475], |
| | | MLWEHNSTIIVMLTK [519], QHGQIR [578], RLNYQTPGMR [606], |
| | | TDEDVPSGPPRK [683], TVDIYGHVTCMR [735], |
| | | VMCVSMGSTTVR [785], YEGVVDMFQTVK [844] |
| 56872 | | AGLGEEFEK [71], AYIATQGPLAESTEDFWR [98], |
| | | DHPPIPITDLADNIER [127], FDLSMPHVQDPSLVR [246], |
| | | FTLTGLKPDTTYDIK [275], GSGPLSPSIQSR [315], |
| | | GVEGSDYINASFLDGYR [326], LNYQTPGMR [475], |
| | | MVWEQR [529], RPPNAWHK [609], SDMGVGVFTPTIEAR [621], |
| | | TVDIYGHVTCMR [735], VLAVNSIGR [774] |
| 105325 | | AAGTEGPFQEVDGVATTR [50], ACNPLDAGPMVVHCSAGVGR [56], |
| | | AYIATQGPLAESTEDFWR [98], DFLPVDPATSNGR [117], |
| | | DINSQQELQNITTDTR [129], FDLSMPHVQDPSLVR [246], |
| | | FEVIEFDDGAGSVLR [249], FTLTGLKPDTTYDIK [275], |
| | | GPPSEAVR [308], GSGPLSPSIQSR [315], |
| | | ILYNGQSVEVDGHSMR [378], MVWEQR [529], |
| | | RPPNAWHK [609], SANYTCVAISSLGMIEATAQVTVK [619], |
| | | SDMGVGVFTPTIEAR [621], TATMLCAAGGNPDPEISWFK [674], |
| | | TATVVMMTR [676], TDEDVPSGPPRK [683], |
| | | TGCFIVIDAMLER [692], TGEGFIDFIGQVHK [693], |
| | | TSVLLSWEVPDSYK [732], VSWVPPPADSR [799] |
| 109891 | | AAGTEGPFQEVDGVATTR [50], ACNPLDAGPMVVHCSAGVGR [56], |
| | | AYIATQGPLAESTEDFWR [98], DDQHFTVTGLHK [111], |
| | | ELPGELLGYR [206], EQFGQDGPITVHCSAGVGR [220], |
| | | FEVIEFDDGAGSVLR [249], ILYNGQSVEVDGHSMRK [379], |
| | | KQNAYIATQGPLPETMGDFWR [433], SDMGVGVFTPTIEAR [621], |
| | | YEGVVDMFQTVK [844] |

b-Cervical cancer

| | | |
|---|---|---|
| 118858 | Integral | AAGTEGPFQEVDGVATTR [50], AHTDVGPGPESSPVLVR [72], |
| | | DDQHFTVTGLHK [111], DFLPVDPATSNGR [117], |
| | | ELPGELLGYR [203], EPMDQKR [218], FEVIEFDDGAGSVLR [249], |
| | | GSSAGGLQHLVSIR [321], QHGQIR [578], RPPNAWHK [609], |
| | | TDEDVPSGPPR [682], TQQGVPAQPADFQAEVESDTR [725], |
| | | TVDIYGHVTCMR [735], VSWVPPPADSR [799], WTEYR [836], |
| | | YSIGGLSPFSEYAFR [872] |
| 124604 | Integral | AAGTEGPFQEVDGVATTR [50], ADEARPNTIDFGK [58], |
| | | AHTDVGPGPESSPVLVR [72], DDQHFTVTGLHK [111], |
| | | ELPGELLGYR [206], EPMDQKR [218], FDLSMPHVQDPSLVR [246], |
| | | FEVIEFDDGAGSVLR [249], GSSAGGLQHLVSIR [321], |
| | | NVLELSNVVR [558], RPPNAWHK [609], SDMGVGVFTPTIEAR [621], |
| | | TQQGVPAQPADFQAEVESDTR [725], VLAVNSIGR [774], |
| | | VSWVPPPADSR [799], VYYTPDSR [823], WTEYR [836], |
| | | YSIGGLSPFSEYAFR [872] |

c-Colorectal cancer

| | |
|---|---|
| Heparin Binding | GSGPLSPSIQSR [315], MAPEPAPGR [509], |
| | SDMGVGVFTPTIEAR [621], TDEDVPSGPPR [682], |
| | TGEQAPSSPPR [694], TQQGVPAQPADFQAEVESDTR [725], |
| | YSAPANLYVR [870] |

d-Hepatocellular carcinoma

| | |
|---|---|
| 72397 | YANVIAYDHSR [840], YSAPANLYVR [870] |

e-Lung cancer

| | |
|---|---|
| | AAGTEGPFQEVDGVATTR [50], ADEARPNTIDFGK [58], |
| | AHTDVGPGPESSPVLVR [72], DSLLAHSSDPVEMR [156], |
| | EHSSWDLVGLEK [196], FEVIEFDDGAGSVLR [249], |
| | GSGPLSPSIQSR [315], GTTYIFR [324], |
| | ILYNGQSVEVDGHSMR [378], ILYNGQSVEVDGHSMRK [379], |
| | KLIADLQPNTEYSFVLMNR [424], |
| | KQNAYIATQGPLPETMGDFWR [433], SGALQIESSEESDQGK [631], |
| | TAPDLLPHKPLPASAYIEDGR [671], |
| | TATMLCAAGGNPDPEISWFK [674], TMPVEQVFAK [708], |
| | TQQGVPAQPADFQAEVESDTR [725], |
| | TQRPAMVQTEDQYQLCYR [726], TVDIYGHVTCMR [735], |
| | YEGVVDMFQTVK [844], YSAPANLYVR [870] |
| 77419 | AYIATQGPLAESTEDFWR [98], DDQHFTVTGLHK [111], |
| | GPPSEAVR [308], GVEGSDYINASFLDGYR [326], |
| | IIMYELVYWAAEDEDQQHK [372], KQNAYIATQGPLPETMGDFWR [433], |
| | LGQVPPGESVTAMELEFK [462], QFQFTDWPEQGVPK [572], |
| | RTHSPSSK [616], YEGVVDMFQTVK [844] |
| 78771 | DHPPIPITDLADNIER [127], DINSQQELQNITTDTR [129], |
| | DSLLAHSSDPVEMRR [157], EHSSWDLVGLEK [196], |
| | GVEGSDYINASFLDGYR [326], ILYNGQSVEVDGHSMRK [379], |
| | LGQVPPGESVTAMELEFK [462], |
| | MLSASTMLVQWEPPEEPNGLVR [518], MLWEHNSTIIVMLTK [519], |
| | MVWEQR [529], QNAYIATQGPLPETMGDFWR [581], |
| | TAPDLLPHKPLPASAYIEDGR [671], TGEQAPSSPPRR [695]], |
| | YANVIAYDHSR [840], YEGVVDMFQTVK [844] |

f-Osteosarcoma

| | |
|---|---|
| 63624 | AAGTEGPFQEVDGVATTR [50], AHTDVGPGPESSPVLVR [72], |
| | AYIATQGPLAESTEDFWR [98], DDQHFTVTGLHK [111], |
| | ELPGELLGYR [206], FDLSMPHVQDPSLVR [246], |
| | FEVIEFDDGAGSVLR [249], FTLTGLKPDTTYDIK [275], |
| | GYQVTYVR [331], ILYNGQSVEVDGHSMR [378], |
| | IQLSWLLPPQER [388], LIADLQPNTEYSFVLMNR [467], |
| | MLSASTMLVQWEPPEEPNGLVR [518], |
| | NGVITQYSVAYEAVDGEDR [537], QHGQIR [578], RPPNAWHK [609], |
| | RRQAER [614], RTHSPSSK [616], SDMGVGVFTPTIEAR [621], |
| | TQQGVPAQPADFQAEVESDTR [725], |
| | VLAFTAVGDGPPSPTIQVK [770], |
| | VPEDQTGLSGGVASFVCQATGEPKPR [789], |
| | VTFDPTSSYTLEDLKPDTLYR [803], YEGVVDMFQTVK [844], |
| | YQYFVVDPMAEYNMPQYILR [869], YSIGGLSPFSEYAFR [872] |

g-Pancreatic cancer

| | |
|---|---|
| 73243 | DDQHFTVTGLHK [111], DSLLAHSSDPVEMRR [157], |
| | GVEGSDYINASFLDGYR [326], LGQVPPGESVTAMELEFK [462], |
| | RLNYQTPGMR [606], TATMLCAAGGNPDPEISWFK [674], |
| | TMPVEQVFAK [708], VGGSMLTPR [764], |
| | YEGVVDMFQTVK [844] |
| 74307 | AYIATQGPLAESTEDFWR [98], DEAIYECTATNSLGEINTSAK [112], |
| | DSLLAHSSDPVEMRR [157], FDLSMPHVQDPSLVR [246], |
| | GPPSEAVR [308], GVEGSDYINASFLDGYR [326], |
| | IIMYELVYWAAEDEDQQHK [372], LNYQTPGMR [475], |
| | MVWEQR [529], SDMGVGVFTPTIEAR [621], TAQSTPSAPPQK [672], |
| | TDEDVPSGPPRK +683+, TGCFIVIDAMLER [692], |
| | YANVIAYDHSR [840], YEGVVDMFQTVK [844], |
| | YSIGGLSPFSEYAFR [872] |
| 115291 | AAGTEGPFQEVDGVATTR [50], DINSQQELQNITTDTR [129], |
| | DSLLAHSSDPVEMRR [157], EHSSWDLVGLEK [196], |
| | EQFGQDGPITVHCSAGVGR [220], FQLAAR [267], |
| | LNYQTPGMR [475], RPPNAWHK [609], RTHSPSSK [616], |
| | SDMGVGVFTPTIEAR [621], TDEDVPSGPPRK [683], |
| | TGCFIVIDAMLER [692], TQQGVPAQPADFQAEVESDTR [725], |
| | YEGVVDMFQTVK [844], YQYFVVDPMAEYNMPQYILR [869], |
| | YSAPANLYVR [870] |
| 124451 | DDQHFTVTGLHK [111], DEAIYECTATNSLGEINTSAK [112], |
| | DFLPVDPATSNGR [117], DHPPIPITDLADNIER [127], |
| | FDLSMPHVQDPSLVR [246], HNTDAGLLTTVGSLLPGITYSLR [341], |
| | LNYQTPGMR [475], MLWEHNSTIIVMLTK [519], |
| | QFQFMAWPDHGVPEYPTPILAFLR [571], RLNYQTPGMR [606], |
| | RTHSPSSK [616], TATVVMMTR [676], TGCFIVIDAMLER [692], |
| | TQRPAMVQTEDQYQLCYR [726], TSVLLSWEVPDSYK [732], |
| | VEVEPLNSTAVHVYWK [756], YEGVVDMFQTVK [844], |
| | YSAPANLYVR [870] |
| 126215 | DDQHFTVTGLHK [111], DSLLAHSSDPVEMR [156], |
| | EHSSWDLVGLEK [196], ELPGELLGYR [206], GTTYIFR [324], |
| | LNYQTPGMR [475], MVWEQR [529], TATVVMMTR [676], |
| | VGGSMLTPR [764], WMMGAEELTK [830], YSAPANLYVR [870] |
| 129890 | AAGTEGPFQEVDGVATTR [50], DSLLAHSSDPVEMR [156], |
| | ELPGELLGYR [206], FEVIEFDDGAGSVLR [249], GPPSEAVR [308], |
| | GSGPLSPSIQSR [315], HVVDGISR [347], LNYQTPGMR [475], |
| | NGVITQYSVAYEAVDGEDR [537], RLNYQTPGMR [606], |
| | RTHSPSSK [616], VSWVPPPADSR [799], YEGVVDMFQTVK [844], |
| | YSAPANLYVR [870], YSIGGLSPFSEYAFR [872] |

h-Renal cell cancer

| | |
|---|---|
| 135833 | AAGTEGPFQEVDGVATTR [50], EHSSWDLVGLEK [196], |
| | FEVIEFDDGAGSVLR [249], FQLAAR [267], |
| | SDMGVGVFTPTIEAR [621], TGEQAPSSPPR [694] |

OGTA002

TABLE 49

| | | |
|---|---|---|
| Samples | | |
| batch no. | Experiment no. | Tryptics identified [SEQ ID No] |

a-Colorectal cancer, iTRAQ

| | | |
|---|---|---|
| Samples 1 | Experiment 1 | AELRGLK [880], TLRLGPLSK [983] |
| Samples 2 | Experiment 1 | ILASVQHMK [373] |
| Samples 2 | Experiment 2 | ILASVQHMK [373] |

b-Kidney cancer, iTRAQ

| | | |
|---|---|---|
| Samples 1 | Experiment 1 | ILASVQHMK [373] |

c-Liver cancer, iTRAQ

| | | |
|---|---|---|
| Samples 1 | Experiment 1 | EIDVSYVK [899] |

d-Non-small cell lung cancer, iTRAQ

| | | |
|---|---|---|
| Samples 1 | Experiment 1 | AELRGLK [880], KCAQLTVNLTR [927] |
| Samples 2 | Experiment 1 | QPHYSAFGSVGEWLRAIK [964] |

OGTA009

TABLE 50

| | | |
|---|---|---|
| Samples batch no. | Experiment no. | Tryptics identified [SEQ ID No] |

a-Colorectal cancer, iTRAQ

| | | |
|---|---|---|
| Samples 1 | Experiment 1 | STGPGASLGTGYDR [657] |
| Samples 1 | Experiment 2 | DFYNPVVPEAQK [119] |
| Samples 2 | Experiment 1 | VYDSLLALPQDLQAAR [817] |
| Samples 2 | Experiment 2 | VYDSLLALPQDLQAAR [817] |
| Samples 2 | Experiment 3 | VYDSLLALPQDLQAAR [817] |
| Samples 2 | Experiment 4 | VYDSLLALPQDLQAAR [817] |

b-Non-small cell lung cancer, iTRAQ

| | | |
|---|---|---|
| Samples 1 | Experiment 1 | VYDSLLALPQDLQAAR [817] |
| Samples 2 | Experiment 1 | VYDSLLALPQDLQAAR [817] |
| Samples 2 | Experiment 2 | VYDSLLALPQDLQAAR [817] |

c-Ovarian cancer, iTRAQ

| | | |
|---|---|---|
| Samples 1 | Experiment 1 | DFYNPVVPEAQK [119] |
| Samples 1 | Experiment 2 | DFYNPVVPEAQK [119] |

OGTA016

TABLE 51

| | | |
|---|---|---|
| Samples batch no. | Experiment no. | Tryptics identified [SEQ ID No] |

a-Colorectal cancer, iTRAQ

| | | |
|---|---|---|
| Samples 1 | Experiment 1 | ADAAPDEK [57], AFVNCDENSR [68], |
| | | ASVDSGSSEEQGGSSR [86], |
| | | LSDAGQYLCQAGDDSNSNKK [947], |
| | | NADLQVLKPEPELVYEDLR [955], |
| | | QSSGENCDVVVNTLGK [588], VYTVDLGR [821] |
| Samples 1 | Experiment 2 | ADAAPDEK [57] |
| Samples 2 | Experiment 1 | ADAAPDEK [57], ASVDSGSSEEQGGSSR [86] |
| Samples 2 | Experiment 2 | AAGSRDVSLAKADAAPDEK [877] |
| Samples 2 | Experiment 3 | ADAAPDEK [57], IIEGEPNLK [371], |
| | | ILLNPQDK [377], VYTVDLGR [821] |
| Samples 2 | Experiment 4 | ASVDSGSSEEQGGSSR [86] |
| Samples 2 | Experiment 5 | ASVDSGSSEEQGGSSR [86] |
| Samples 2 | Experiment 6 | ASVDSGSSEEQGGSSR [86], IIEGEPNLK [371], |
| | | ILLNPQDK [377], QSSGENCDVVVNTLGK [588] |

b-Non-small cell lung cancer, iTRAQ

| | | |
|---|---|---|
| Samples 1 | Experiment 1 | ADAAPDEK [57], ASVDSGSSEEQGGSSR [86], |
| | | ILLNPQDK [377] |
| Samples 2 | Experiment 1 | IIEGEPNLK [371] |
| Samples 2 | Experiment 2 | ESKSIK [907], TVTINCPFK [987] |

OGTA028

TABLE 52

| | | |
|---|---|---|
| Samples batch no. | Experiment no. | Tryptics identified [SEQ ID No] |

a-Colorectal cancer, iTRAQ

| | | |
|---|---|---|
| Samples 1 | Experiment 1 | AEDIIK [879], DIAPVLDLK [890] |
| Samples 2 | Experiment 1 | DIAPVLDLK [890] |
| Samples 2 | Experiment 2 | DIAPVLDLK [890], EEINQGGR [177], IDEKR [919] |
| Samples 2 | Experiment 3 | DIAPVLDLK [890], SSVKELLK [973] |
| Samples 2 | Experiment 4 | DIAPVLDLK [890] |
| Samples 2 | Experiment 5 | DIAPVLDLK [890] |
| Samples 2 | Experiment 6 | DIAPVLDLK [890] |

b-Kidney cancer, iTRAQ

| | | |
|---|---|---|
| Samples 1 | Experiment 1 | DIAPVLDLK [890], EEINQGGR [177] |
| Samples 1 | Experiment 2 | DIAPVLDLK [890], EEINQGGR [177] |
| Samples 1 | Experiment 3 | DIAPVLDLK [890] |

c-Liver cancer, iTRAQ

| | | |
|---|---|---|
| Samples 1 | Experiment 1 | VKTTSLTEKK [992] |
| Samples 1 | Experiment 2 | DIAPVLDLK [890], WSNVFK [832] |

d-Non-small cell lung cancer, iTRAQ

| | | |
|---|---|---|
| Samples 1 | Experiment 1 | DIAPVLDLK [890], LKNKEEVLK [933], VLLEK [995] |
| Samples 1 | Experiment 2 | DIAPVLDLK [890] |
| Samples 2 | Experiment 1 | DIAPVLDLK [890] |
| Samples 2 | Experiment 2 | DIAPVLDLK [890] |
| Samples 2 | Experiment 3 | DIAPVLDLK [890] |
| Samples 2 | Experiment 4 | DIAPVLDLK [890], DVLPQK [897] |
| Samples 2 | Experiment 5 | DIAPVLDLK [890], EEINQGGR [177] |

e-Small cell lung cancer, iTRAQ

| | | |
|---|---|---|
| Samples 1 | Experiment 1 | DIAPVLDLK [890] |
| Samples 1 | Experiment 2 | DIAPVLDLK [890], EEINQGGR [177] |

f-Ovarian cancer, iTRAQ

| | | |
|---|---|---|
| Samples 1 | Experiment 1 | DIAPVLDLK [890] |
| Samples 1 | Experiment 2 | DIAPVLDLK [890] |
| Samples 1 | Experiment 3 | DIAPVLDLK [890] |
| Samples 1 | Experiment 4 | DIAPVLDLK [890] |

OGTA037

TABLE 53

| | | |
|---|---|---|
| Samples batch | | Tryptics identified |
| no. | Experiment no. | [SEQ ID No] |

a-Colorectal cancer, iTRAQ

| | | |
|---|---|---|
| Samples 1 | Experiment 1 | VLAANNVR [993] |

b-Non-small cell lung cancer, iTRAQ

| | | |
|---|---|---|
| Samples 1 | Experiment 1 | VLAANNVR [993] |

c-Ovarian cancer, iTRAQ

| | | |
|---|---|---|
| Samples 1 | Experiment 1 | VLAANNVR [993] |

OGTA041

TABLE 54
Non-small cell lung cancer, iTRAQ

| | | Tryptics identified |
|---|---|---|
| Sample no. | Experiment no. | [SEQ ID No] |
| Samples 1 | Experiment 1 | LTWTNPSIK [950] |
| Samples 2 | Experiment 1 | LTWTNPSIK [950] |

OGTA053

TABLE 55

| | | |
|---|---|---|
| Samples batch no. | Experiment no. | Tryptics identified [SEQ ID No] |

a-Colorectal cancer, iTRAQ

| | | |
|---|---|---|
| Samples 1 | Experiment 1 | VVISIR [1002] |
| Samples 2 | Experiment 1 | TELLAADSLSK [980] |
| Samples 2 | Experiment 2 | VVISIR [1002] |
| Samples 2 | Experiment 3 | IRTSTPTGHGASPAK [924] |
| Samples 2 | Experiment 4 | TELLAADSLSK [980] |
| Samples 2 | Experiment 5 | IRTSTPTGHGASPAK [924], TELLAADSLSK [980] |

b-Kidney cancer, iTRAQ

| | | |
|---|---|---|
| Samples 1 | Experiment 1 | TELLAADSLSK [980] |
| Samples 1 | Experiment 2 | IRTSTPTGHGASPAK [924], TELLAADSLSK [980] |
| Samples 1 | Experiment 3 | IRTSTPTGHGASPAK [924] |

c-Liver cancer, iTRAQ

| | | |
|---|---|---|
| Samples 1 | Experiment 1 | IRTSTPTGHGASPAK [924] |

d-Non-small cell lung cancer, iTRAQ

| | | |
|---|---|---|
| Samples 1 | Experiment 1 | IRTSTPTGHGASPAK [924], TELLAADSLSK [980] |
| Samples 1 | Experiment 2 | GTLSPKDALTDLTGDAER [916], IRTSTPTGHGASPAK |
| | | [924], TELLAADSLSK [980] |
| Samples 2 | Experiment 1 | IRTSTPTGHGASPAK [924] |
| Samples 2 | Experiment 2 | IRTSTPTGHGASPAK [924], TELLAADSLSK [980] |

e-Small cell lung cancer, iTRAQ

| | | |
|---|---|---|
| Samples 1 | Experiment 1 | TELLAADSLSK [980] |
| Samples 1 | Experiment 2 | TELLAADSLSK [980] |

OGTA054

TABLE 56
a - Colorectal cancer, iTRAQ

| Samples batch no. | Experiment no. | Tryptics identified [SEQ ID No] |
|---|---|---|
| Samples 1 | Experiment 1 | EEHEVAVLGAPPSTILPR [173], |
| | | MVGDVTGAQAYASTAK [954] |
| Samples 2 | Experiment 1 | MVGDVTGAQAYASTAK [954] |

b - Non-small cell lung cancer, iTRAQ

| Sample no. | Experiment no. | Tryptics identified [SEQ ID No] |
|---|---|---|
| Samples 1 | Experiment 1 | MVGDVTGAQAYASTAK [954] |
| Samples 1 | Experiment 2 | MVGDVTGAQAYASTAK [954] |

OGTA066

TABLE 57

| | | |
|---|---|---|
| Samples batch no. | Experiment no. | Tryptics identified [SEQ ID No] |

a-Colorectal cancer iTRAQ

| | | |
|---|---|---|
| Samples 1 | Experiment 1 | IMFVDPSLTVR [380] [3] |
| Samples 1 | Experiment 2 | IMFVDPSLTVR [380] [3] |
| Samples 2 | Experiment 1 | IMFVDPSLTVR [380] [3] |
| Samples 2 | Experiment 2 | DLPLLIENMK [135] [2], IMFVDPSLTVR [380] [3] |
| Samples 2 | Experiment 3 | IMFVDPSLTVR [380] [3] |
| Samples 2 | Experiment 4 | DLPLLIENMK [135] [2], IMFVDPSLTVR [380] [3] |
| Samples 2 | Experiment 5 | IMFVDPSLTVR [380] [3] |

b-Kidney cancer iTRAQ

| | | |
|---|---|---|
| Samples 1 | Experiment 1 | DLPLLIENMK [135] [2] |

c-Non-small cell lung cancer iTRAQ

| | | |
|---|---|---|
| Samples 1 | Experiment 1 | DLPLLIENMK [135] [2], IMFVDPSLTVR [380] [3], |
| | | NLSPDGQYVPR [546] [5] |
| Samples 2 | Experiment 1 | IMFVDPSLTVR [380] [3] |
| Samples 2 | Experiment 2 | IMFVDPSLTVR [380] [3] |
| Samples 2 | Experiment 3 | IMFVDPSLTVR [380] [3] |
| Samples 2 | Experiment 4 | IMFVDPSLTVR [380] [3] |

OGTA074

TABLE 58
Non-small cell lung cancer, iTRAQ

| | | Tryptics identified] |
|---|---|---|
| Sample no. | Experiment no. | [SEQ ID No] |
| Samples 1 | Experiment 1 | CLNPVPAVPSPSPSVRK [888] |

OGTA076

TABLE 59

| | | |
|---|---|---|
| Samples batch no. | Experiment no. | Tryptics identified [SEQ ID No] |

a-Colorectal cancer iTRAQ

| | | |
|---|---|---|
| Samples 1 | Experiment 1 | EGIAK [898] [14], IIPK [921] [28], LALK [929] [35], |
| | | LNPK [941] [40] |
| Samples 1 | Experiment 2 | EGIAK [898] [14], LALK [929] [35] |
| Samples 1 | Experiment 3 | EGIAK [898] [14], IIPK [921] [28], LALK [929] [35] |
| Samples 1 | Experiment 4 | LNPK [941] [40] |

b-Kidney cancer iTRAQ

| | | |
|---|---|---|
| Samples 1 | Experiment 1 | EGIAK [898] [14], LALK [929] [35], LNPK [941] [40], |
| | | YLNNLYK [1006] [66] |
| Samples 1 | Experiment 2 | LALK [929] [35] |

c-Liver cancer iTRAQ

| | | |
|---|---|---|
| Samples 1 | Experiment 1 | YLNNLYK [1006] [66] |

d-Non-small cell lung cancer iTRAQ

| | | |
|---|---|---|
| Samples 1 | Experiment 1 | YLNNLYK [1006] [66] |
| Samples 1 | Experiment 2 | LALK [929] [35] |
| Samples 2 | Experiment 1 | IIPK [921] [28], LALK [929] [35], YLNNLYK [1006] [66] |
| Samples 2 | Experiment 2 | YLNNLYK [1006] [66] |

e-Small cell lung cancer iTRAQ

| | | |
|---|---|---|
| Samples 1 | Experiment 1 | EGIAK [898] [14] |
| Samples 1 | Experiment 2 | EGIAK [898] [14] |

f-Ovarian cancer iTRAQ

| | | |
|---|---|---|
| Samples 1 | Experiment 1 | LALK [929] [35] |
| Samples 1 | Experiment 2 | LALK [929] [35], YLNNLYK [1006] [66] |
| Samples 1 | Experiment 3 | LALK [929] [35] |

OGTA085

TABLE 60

| | | |
|---|---|---|
| Samples batch | | Tryptics identified |
| no. | Experiment no. | [SEQ ID No] |

a-Colorectal cancer, iTRAQ

| | | |
|---|---|---|
| Samples 1 | Experiment 1 | KMLGNPSR [427] |

b-Non-small cell lung cancer, iTRAQ

| | | |
|---|---|---|
| Samples 1 | Experiment 1 | KMLGNPSR [427] |

OGTA088

TABLE 61

| | | |
|---|---|---|
| Samples batch | | Tryptics identified |
| no. | Experiment no. | [SEQ ID No] |

a-Colorectal cancer, iTRAQ

| | | |
|---|---|---|
| Samples 1 | Experiment 1 | RQEELERK [613] |

b-Non-small cell lung cancer, iTRAQ

| | | |
|---|---|---|
| Samples 1 | Experiment 1 | RQEELERK [613] |

OGTA089

TABLE 62

| | | |
|---|---|---|
| | | Tryptics identified |
| Sample no. | Experiment no. | [SEQ ID No] |

a-Kidney cancer, iTRAQ

| | | |
|---|---|---|
| Samples 1 | Experiment 1 | GHEVAAMLK [913] |

b-Non-small cell lung cancer, iTRAQ

| | | |
|---|---|---|
| Samples 1 | Experiment 1 | LLHGQNGSVPNGQTPLK [935] |

c-Ovarian cancer, iTRAQ

| | | |
|---|---|---|
| Samples 1 | Experiment 1 | NNLVEIILDINVSQLTER [956] |

OGTA091

TABLE 63

| | | |
|---|---|---|
| Samples batch no. | Experiment no. | Tryptics identified [SEQ ID No] |

a-Colorectal cancer, iTRAQ

| | | |
|---|---|---|
| Samples 1 | Experiment 1 | IYIVR [926] |
| Samples 2 | Experiment 1 | KRSAPTSR [928] |

b-Kidney cancer, iTRAQ

| | | |
|---|---|---|
| Samples 1 | Experiment 1 | LLSDK [938], RMEPLEK [607] |
| Samples 1 | Experiment 2 | FSDVTGKIK [912], IGETVVDLENR [367], |
| | | RRDLSQMEALK [966] |

c-Liver cancer, iTRAQ

| | | |
|---|---|---|
| Samples 1 | Experiment 1 | VVFQIWDNDK [810] |

d-Non-small cell lung cancer, iTRAQ

| | | |
|---|---|---|
| Samples 1 | Experiment 1 | IPNPHLGPVEER [386], IYIVR [926], TAIEILAWGLR |
| | | [976] |
| Samples 1 | Experiment 2 | FSDVTGK [272], VTAAGQTK [800] |
| Samples 2 | Experiment 1 | RRDLSQMEALK [966] |

e-Small cell lung cancer, iTRAQ

| | | |
|---|---|---|
| Samples 1 | Experiment 1 | LLSDK [938] |

f-Ovarian cancer, iTRAQ

| | | |
|---|---|---|
| Samples 1 | Experiment 1 | VTAAGQTKRTR [999] |
| Samples 1 | Experiment 2 | VPAHQVLFSRR [997], VTAAGQTKRTR [999] |

OGTA098

TABLE 64

| | | |
|---|---|---|
| Samples batch no. | Experiment no. | Tryptics identified [SEQ ID No] |

a-Colorectal cancer, iTRAQ

| | | |
|---|---|---|
| Samples 1 | Experiment 1 | SLQEANAEK [970], TLAEVCLGQK [982] |
| Samples 2 | Experiment 1 | INATDADEPNTLNSK [381], SLQEANAEK [970] |

b-Kidney cancer, iTRAQ

| | | |
|---|---|---|
| Samples 1 | Experiment 1 | SLQEANAEK [970] |

c-Non-small cell lung cancer, iTRAQ

| | | |
|---|---|---|
| Samples 1 | Experiment 1 | INATDADEPNTLNSK [381] |
| Samples 1 | Experiment 2 | NPIAK [957] |
| Samples 2 | Experiment 1 | NPIAK [957] |

d-Small cell lung cancer, iTRAQ

| | | |
|---|---|---|
| Samples 1 | Experiment 1 | NPIAK [957], TARATGASR [977] |

e-Ovarian cancer, iTRAQ

| | | |
|---|---|---|
| Samples 1 | Experiment 1 | NPIAK [957] |

OGTA101

TABLE 65

| | | |
|---|---|---|
| Samples batch no. | Experiment no. | Tryptics identified [SEQ ID No] |

a-Colorectal cancer, iTRAQ

| | | |
|---|---|---|
| Samples 1 | Experiment 1 | LSARNK [946] |
| Samples 2 | Experiment 1 | IKQLR [922] |
| Samples 2 | Experiment 2 | LASSKAHTSR [930] |

b-Kidney cancer, iTRAQ

| | | |
|---|---|---|
| Samples 1 | Experiment 1 | ILYDDGKMVEEVDGR [923] |

c-Liver cancer, iTRAQ

| | | |
|---|---|---|
| Samples 1 | Experiment 1 | IKQLR [922] |

d-Non-small cell lung cancer, iTRAQ

| | | |
|---|---|---|
| Samples 1 | Experiment 1 | ELREVR [901], LTVLR [949], MRYEGVVDIFQTVK |
| | | [952], VEVEAVNSTSVK [755] |
| Samples 1 | Experiment 2 | LASSKAHTSR [930], LTVLR [949] |
| Samples 2 | Experiment 1 | GNSAGGLQHRVTAK [914], LSARNK [946] |
| Samples 2 | Experiment 2 | TVDIYGHVTLMR [736] |

e-Ovarian cancer, iTRAQ

| | | |
|---|---|---|
| Samples 1 | Experiment 1 | YEGVVDIFQTVK [843] |

OGTA104

TABLE 66

| | | |
|---|---|---|
| Samples batch no. | Experiment no. | Tryptics identified [SEQ ID No] |

a-Colorectal cancer, iTRAQ

| | | |
|---|---|---|
| Samples 1 | Experiment 1 | FPSGTLR [264], NQTAVR [959] |
| Samples 2 | Experiment 1 | LQCENVQEIPVFGIVPAIIQTPSR [943] |
| Samples 2 | Experiment 2 | FPSGTLR [264] |

b-Kidney cancer, iTRAQ

| | | |
|---|---|---|
| Samples 1 | Experiment 1 | FLITRRR [910] |

c-Liver cancer, iTRAQ

| | | |
|---|---|---|
| Samples 1 | Experiment 1 | SQTYESDGK [972] |

d-Non-small cell lung cancer, iTRAQ

| | | |
|---|---|---|
| Samples 1 | Experiment 1 | FLITRRR [910] |
| Samples 1 | Experiment 2 | CGVSNKDIEK [887], FLITRRR [910] |

e-Ovarian cancer, iTRAQ

| | | |
|---|---|---|
| Samples 1 | Experiment 1 | FLITRRR [910] |

OGTA106

TABLE 67
Liver cancer, iTRAQ

| | | Tryptics identified |
|---|---|---|
| Sample no. | Experiment no. | [SEQ ID No] |
| Samples 1 | Experiment 1 | EVCQLLLR [908] |

OGTA112

TABLE 68
Kidney cancer, iTRAQ

| | | Tryptics identified |
|---|---|---|
| Sample no. | Experiment no. | [SEQ ID No] |
| Samples 1 | Experiment 1 | LFGEK [932] |

OGTA113

TABLE 69

| | | |
|---|---|---|
| Samples batch no. | Experiment no. | Tryptics identified [SEQ ID No] |

a-Colorectal cancer, iTRAQ

| | | |
|---|---|---|
| Samples 1 | Experiment 1 | LLSDPNYGVHLPAVK [939] |
| Samples 2 | Experiment 1 | YPEVGDLR [865] |
| Samples 2 | Experiment 2 | LEDPHVDIIRR [931] |

b-Kidney cancer, iTRAQ

| | | |
|---|---|---|
| Samples 1 | Experiment 1 | LEDPHVDIIR [449] |

c-Non-small cell lung cancer, iTRAQ

| | | |
|---|---|---|
| Samples 1 | Experiment 1 | YPEVGDLR [865], YSYNTEWR [874] |

d-Ovarian cancer, iTRAQ

| | | |
|---|---|---|
| Samples 1 | Experiment 1 | LEDPHVDIIR [449] |

OGTA119

TABLE 70

| | | |
|---|---|---|
| Samples batch no. | Experiment no. | Tryptics identified [SEQ ID No] |

a-Colorectal cancer, iTRAQ

| | | |
|---|---|---|
| Samples 1 | Experiment 1 | ALALLEDEER [75], GEGPEAGAGGAGGR [290] |
| Samples 2 | Experiment 1 | VLTDIK [996] |
| Samples 2 | Experiment 2 | MTPPSRAEAGVRR [953] |

b-Non-small cell lung cancer, iTRAQ

| | | |
|---|---|---|
| Samples 1 | Experiment 1 | AEWLNK [64], DTYLK [896] |
| Samples 1 | Experiment 2 | GEGPEAGAGGAGGR [290] |
| Samples 2 | Experiment 1 | DTYLK [896] |

c-Ovarian cancer, iTRAQ

| | | |
|---|---|---|
| Samples 1 | Experiment 1 | ALALLEDEER [75] |
| Samples 1 | Experiment 2 | ALALLEDEER [75] |

OGTA124

TABLE 71

| | | |
|---|---|---|
| Samples batch no. | Experiment no. | Tryptics identified [SEQ ID No] |

a-Colorectal cancer, iTRAQ

| | | |
|---|---|---|
| Samples 1 | Experiment 1 | LLQAIAK [937] |

b-Kidney cancer, iTRAQ

| | | |
|---|---|---|
| Samples 1 | Experiment 1 | IFTVAQMDDNSIQMKK [365], LDEDLHVK [446] |

c-Liver cancer, iTRAQ

| | | |
|---|---|---|
| Samples 1 | Experiment 1 | EWGDGIR [240] |

d-Non-small cell lung cancer, iTRAQ

| | | |
|---|---|---|
| Samples 1 | Experiment 1 | LDEDLHVK [446] |
| Samples 1 | Experiment 2 | LDEDLHVK [446] |

e-Ovarian cancer, iTRAQ

| | | |
|---|---|---|
| Samples 1 | Experiment 1 | EREAQLVK [905] |

OGTA126

TABLE 72
Non-small cell lung cancer, iTRAQ

| Sample no. | Experiment no. | Tryptics identified [SEQ ID No] |
|---|---|---|
| Samples 1 | Experiment 1 | IGTFCSNGTVSRIK [1007], LLVPKDR [940], LWMNVEK |
| | | [505] |
| Samples 2 | Experiment 1 | LLVPKDR [940] |

OGTA156

TABLE 73

| | | |
|---|---|---|
| Samples batch no. | Experiment no. | Tryptics identified [SEQ ID No] |

a-Colorectal cancer, iTRAQ

| | | |
|---|---|---|
| Samples 1 | Experiment 1 | DIMKKTINFAR [891] |

b-Kidney cancer, iTRAQ

| | | |
|---|---|---|
| Samples 1 | Experiment 1 | DIMKKTINFAR [891], HSRVTVAIPLK [918] |

c-Non-small cell lung cancer, iTRAQ

| | | |
|---|---|---|
| Samples 1 | Experiment 1 | HSRVTVAIPLK [918] |
| Samples 2 | Experiment 1 | HSRVTVAIPLK [918], TYLAVGSMK [740] |

d-Ovarian cancer, iTRAQ

| | | |
|---|---|---|
| Samples 1 | Experiment 1 | DIMKKTINFAR [891] |

OGTA159

TABLE 74

| | | |
|---|---|---|
| | | Tryptics identified |
| Sample no. | Experiment no. | [SEQ ID No] |

a-Kidney cancer, iTRAQ

| | | |
|---|---|---|
| Samples 1 | Experiment 1 | RDLGSRLQAQR [965] |
| Samples 1 | Experiment 2 | RDLGSRLQAQR [965] |

b-Non-small cell lung cancer, iTRAQ

| | | |
|---|---|---|
| Samples 1 | Experiment 1 | RDLGSRLQAQR [965] |
| Samples 1 | Experiment 2 | RDLGSRLQAQR [965] |

OGTA169

TABLE 75

| | | |
|---|---|---|
| Samples batch no. | Experiment no. | Tryptics identified [SEQ ID No] |

a-Colorectal cancer, iTRAQ

| | | |
|---|---|---|
| Samples 1 | Experiment 1 | GGQVSVCPLPR [299], NRSLSRVR [961] |
| Samples 2 | Experiment 1 | GGQVSVCPLPR [299] |
| Samples 2 | Experiment 2 | TSKTTFQLELPVK [985] |

b-Kidney cancer, iTRAQ

| | | |
|---|---|---|
| Samples 1 | Experiment 1 | AVLGDR [885] |

c-Non-small cell lung cancer, iTRAQ

| | | |
|---|---|---|
| Samples 1 | Experiment 1 | GGQVSVCPLPR [299] |
| Samples 2 | Experiment 1 | TSKTTFQLELPVK [985] |
| Samples 2 | Experiment 2 | GGQVSVCPLPR [299], LTDVVIGAPGEEENR [496], |
| | | YFTASLPFEK [1004] |

OGTA174

TABLE 76
Non-small cell lung cancer, iTRAQ

| | | Tryptics identified |
|---|---|---|
| Sample no. | Experiment no. | [SEQ ID No] |
| Samples 1 | Experiment 1 | LSQCHELWER [490] |

OGTA176

TABLE 77

| | | |
|---|---|---|
| Samples batch | | Tryptics identified |
| no. | Experiment no. | [SEQ ID No] |

a-Colorectal cancer, iTRAQ

| | | |
|---|---|---|
| Samples 1 | Experiment 1 | AVTSPAVGR [886] |
| Samples 1 | Experiment 2 | AVTSPAVGR [886] |

b-Kidney cancer, iTRAQ

| | | |
|---|---|---|
| Samples 1 | Experiment 1 | AVTSPAVGR [886] |

c-Non-small cell lung cancer, iTRAQ

| | | |
|---|---|---|
| Samples 1 | Experiment 1 | AVTSPAVGR [886] |

d-Small cell lung cancer, iTRAQ

| | | |
|---|---|---|
| Samples 1 | Experiment 1 | AVTSPAVGR [886] |
| Samples 1 | Experiment 2 | AVTSPAVGR [886] |

OGTA177

TABLE 78

| | | |
|---|---|---|
| Samples batch no. | Experiment no. | Tryptics identified [SEQ ID No] |

a-Colorectal cancer, iTRAQ

| | | |
|---|---|---|
| Samples 1 | Experiment 1 | VLDVVER [994], VVNVSDLYK [1003] |
| Samples 2 | Experiment 1 | AREVIPR [883] |
| Samples 2 | Experiment 2 | DIQVASNEILR [131], SQHQETPVYLGATAGMRLLR |
| | | [971] |
| Samples 2 | Experiment 3 | GPGISK [915], SQHQETPVYLGATAGMRLLR [971] |

b-Kidney cancer, iTRAQ

| | | |
|---|---|---|
| Samples 1 | Experiment 1 | DIQVASNEILR [131], GPGISK [915],SLSNYPFDFQGAR |
| | | [643], VLDVVER [994] |

c-Non-small cell lung cancer, iTRAQ

| | | |
|---|---|---|
| Samples 1 | Experiment 1 | DIQVASNEILR [131], DPCFHPGYK [892], |
| | | SLSNYPFDFQGAR [643], SQHQETPVYLGATAGMR |
| | | [647] |
| Samples 1 | Experiment 2 | EVIPR [909], SLSNYPFDFQGAR [643] |
| Samples 2 | Experiment 1 | FLNLTSEKVSQEK [911] |

d-Small cell lung cancer, iTRAQ

| | | |
|---|---|---|
| Samples 1 | Experiment 1 | AREVIPR [883] |

e-Ovarian cancer, iTRAQ

| | | |
|---|---|---|
| Samples 1 | Experiment 1 | GPGISK [915] |

OGTA197

TABLE 79

| | | |
|---|---|---|
| Samples batch no. | Experiment no. | Tryptics identified [SEQ ID No] |

a-Colorectal cancer, iTRAQ

| | | |
|---|---|---|
| Samples 1 | Experiment 1 | LNVEERSVGPLTR [942] |
| Samples 2 | Experiment 1 | NGVSGLVTSR [538], TASVSINQTEPPK [673] |
| Samples 2 | Experiment 2 | NGVSGLVTSR [538] |

b-Kidney cancer, iTRAQ

| | | |
|---|---|---|
| Samples 1 | Experiment 1 | LPGHQKR [478], NGVSGLVTSR [538], |
| | | TASVSINQTEPPK [673] |

c-Liver cancer, iTRAQ

| | | |
|---|---|---|
| Samples 1 | Experiment 1 | TASVSINQTEPPK [673] |

d-Non-small cell lung cancer, iTRAQ

| | | |
|---|---|---|
| Samples 1 | Experiment 1 | TASVSINQTEPPK [673] |
| Samples 1 | Experiment 2 | DGEFSVLQLVGMLR [121], LNVEERSVGPLTR [942] |
| Samples 2 | Experiment 1 | NGVSGLVTSR [538] |

e-Small cell lung cancer, iTRAQ

| | | |
|---|---|---|
| Samples 1 | Experiment 1 | SVGPLTRK [663], TASVSINQTEPPK [673] |
| Samples 1 | Experiment 2 | NGVSGLVTSR [538], SVGPLTRK [663], |
| | | TASVSINQTEPPK [673] |

OGTA202

TABLE 80

| | | |
|---|---|---|
| Samples batch no. | Experiment no. | Tryptics identified [SEQ ID No] |

a-Colorectal cancer, iTRAQ

| | | |
|---|---|---|
| Samples 1 | Experiment 1 | STGTISVISSGLDREK [975] |
| Samples 2 | Experiment 1 | EQLTVIR [904], ISYRILR [925] |

b-Non-small cell lung cancer, iTRAQ

| | | |
|---|---|---|
| Samples 1 | Experiment 1 | EQLTVIR [904] |
| Samples 1 | Experiment 2 | EQLTVIR [904] |
| Samples 2 | Experiment 1 | EQLTVIR [904] |

OGTA203

TABLE 81

| | | |
|---|---|---|
| | | Tryptics identified |
| Sample no. | Experiment no. | [SEQ ID No] |

a-Kidney cancer, iTRAQ

| | | |
|---|---|---|
| Samples 1 | Experiment 1 | LLDFEK [934], |
| | | TGRPVEPESEFIIK [981] |

b-Non-small cell lung cancer, iTRAQ

| | | |
|---|---|---|
| Samples 1 | Experiment 1 | VEASNPYVEPRFLYLGPFK [989] |

OGTA206

TABLE 82
a - Colorectal cancer, iTRAQ

| Samples batch no. | Experiment no. | Tryptics identified [SEQ ID No] |
|---|---|---|
| Samples 1 | Experiment 1 | VYDSLLALPQDLQAAR [817] |
| Samples 2 | Experiment 1 | SAAASNYV [968], |
| | | VYDSLLALPQDLQAAR [817] |
| Samples 2 | Experiment 2 | VYDSLLALPQDLQAAR [817] |
| Samples 2 | Experiment 3 | VYDSLLALPQDLQAAR [817] |
| Samples 2 | Experiment 4 | VYDSLLALPQDLQAAR [817] |

b - Non-small cell lung cancer, iTRAQ

| Sample no. | Experiment no. | Tryptics identified [SEQ ID No] |
|---|---|---|
| Samples 1 | Experiment 1 | VYDSLLALPQDLQAAR [817] |
| Samples 2 | Experiment 1 | VYDSLLALPQDLQAAR [817] |
| Samples 2 | Experiment 2 | VYDSLLALPQDLQAAR [817] |

OGTA213

TABLE 83
a - Colorectal cancer, iTRAQ

| Samples batch no. | Experiment no. | Tryptics identified [SEQ ID No] |
|---|---|---|
| Samples 1 | Experiment 1 | RSSAAGEGTLAR [967] |
| Samples 2 | Experiment 1 | RSSAAGEGTLAR [967] |
| Samples 2 | Experiment 2 | RSSAAGEGTLAR [967] |

b - Non-small cell lung cancer, iTRAQ

| Sample no. | Experiment no. | Tryptics identified [SEQ ID No] |
|---|---|---|
| Samples 1 | Experiment 1 | RSSAAGEGTLAR [967] |
| Samples 1 | Experiment 2 | RSSAAGEGTLAR [967] |
| Samples 2 | Experiment 1 | RSSAAGEGTLAR [967] |

OGTA214

TABLE 84
Colorectal cancer, iTRAQ

| Samples batch no. | Experiment no. | Tryptics identified [SEQ ID No] |
|---|---|---|
| Samples 1 | Experiment 1 | DSQMQNPYSR [895], |
| | | HIEEENLIDEDFQNLK [917], |
| | | SDLQRPNPQSPFCVASSLK [969], |
| | | STGFTNLGAEGSVFPK [974] |
| Samples 1 | Experiment 2 | STGFTNLGAEGSVFPK [974] |
| Samples 2 | Experiment 1 | HIEEENLIDEDFQNLK [917] |
| Samples 2 | Experiment 2 | VFPGK [990] |

OGTA216

TABLE 85
a - Colorectal cancer, iTRAQ

| Samples batch no. | Experiment no. | Tryptics identified [SEQ ID No] |
|---|---|---|
| Samples 1 | Experiment 1 | AAAAAAAAAAAAAAAGAGAGAK [49], |
| | | AFYAPVHADDLR [69], GGGAYYLISR [298], |
| | | GPIVPLNVADQK [307], ITDNELELYK [395] |
| Samples 1 | Experiment 2 | DLPPILLVR [136], EGAQYLMQAAGLGR [186] |
| Samples 2 | Experiment 1 | AMATLLSK [78], EGAQYLMQAAGLGR [186] |
| Samples 2 | Experiment 2 | DLPPILLVR [136] |
| Samples 2 | Experiment 3 | QTPADGEASGESEPAK [1008] |

b - Kidney cancer, iTRAQ

| Sample no. | Experiment no. | Tryptics identified [SEQ ID No] |
|---|---|---|
| Samples 1 | Experiment 1 | RAMATLLSK [596] |
| Samples 1 | Experiment 2 | RAMATLLSK [596], SPGWRPAFK [645] |

c - Liver cancer, iTRAQ

| Sample no. | Experiment no. | Tryptics identified [SEQ ID No] |
|---|---|---|
| Samples 1 | Experiment 1 | IFQALCK [920] |
| Samples 1 | Experiment 2 | SPGWRPAFK [645] |

d - Non-small cell lung cancer, iTRAQ

| Sample no. | Experiment no. | Tryptics identified [SEQ ID No] |
|---|---|---|
| Samples 1 | Experiment 1 | AMATLLSK [78], DCKIRVFIGGK [889], |
| | | TATQPLLK [978] |
| Samples 1 | Experiment 2 | EMSIDQAK [213], LHEDDK [465] |
| Samples 2 | Experiment 1 | EQDIADK [903] |
| Samples 2 | Experiment 2 | DLPPILLVR [136] |

e - Small cell lung cancer, iTRAQ

| Sample no. | Experiment no. | Tryptics identified [SEQ ID No] |
|---|---|---|
| Samples 1 | Experiment 1 | LSGVEDHVK [948] |

f - Ovarian cancer, iTRAQ

| Sample no. | Experiment no. | Tryptics identified [SEQ ID No] |
|---|---|---|
| Samples 1 | Experiment 1 | AMATLLSK [78] |

OGTA222

TABLE 86
a - Colorectal cancer, iTRAQ

| Samples batch no. | Experiment no. | Tryptics identified [SEQ ID No] |
|---|---|---|
| Samples 1 | Experiment 1 | AGFVGGQFWSVYTPCDTQNK [881], ATLQLSR [91], |
| | | AVGFGGDFDGVPR [95], GALADNLLR [283], NVPDDVLR |
| | | [559], TLEQMDVVHR [703] |
| Samples 1 | Experiment 2 | AGFVGGQFWSVYTPCDTQNK [881], ALYQLGMR [77], |
| | | AVGFGGDFDGVPR [95], DSPVIDGHNDLPWQLLDMFNNR |
| | | [894]. GALADNLLR [283], TLEQMDVVHR [703], |
| | | VASLIGVEGGHSIDSSLGVLR [988], VPEGLEDVSK [791], |
| | | YPDLIAELLR [864] |
| Samples 2 | Experiment 1 | AVGFGGDFDGVPR [95] |

b - Kidney cancer, iTRAQ

| Sample no. | Experiment no. | Tryptics identified [SEQ ID No] |
|---|---|---|
| Samples 1 | Experiment 1 | AVGFGGDFDGVPR [95], VPEGLEDVSK [791] |
| Samples 1 | Experiment 2 | ALYQLGMR [77], AVGFGGDFDGVPR [95], GALADNLLR [283], |
| | | NVPDDVLR [559], VPEGLEDVSK [791], YPDLIAELLR [864] |
| Samples 1 | Experiment 3 | AVGFGGDFDGVPR [95] |

c - Non-small cell lung cancer, iTRAQ

| Sample no. | Experiment no. | Tryptics identified [SEQ ID No] |
|---|---|---|
| Samples 1 | Experiment 1 | ANLSQVADHLDHIK [882] |
| Samples 1 | Experiment 2 | VPEGLEDVSK [791] |

OGTA236

TABLE 87
Colorectal cancer, iTRAQ

| Samples | | Tryptics identified |
|---|---|---|
| batch no. | Experiment no. | [SEQ ID No] |
| Samples 1 | Experiment 1 | ELNEHFKSK [900] |

OGTA237

TABLE 88
a - Colorectal cancer, iTRAQ

| Samples batch no. | Experiment no. | Tryptics identified [SEQ ID No] |
|---|---|---|
| Samples 1 | Experiment 1 | QLVEALDK [963] |

b - Kidney cancer, iTRAQ

| Sample no. | Experiment no. | Tryptics identified [SEQ ID No] |
|---|---|---|
| Samples 1 | Experiment 1 | AEAFGMDPARPDQASTVAVK [878] |

c - Non-small cell lung cancer, iTRAQ

| Sample no. | Experiment no. | Tryptics identified [SEQ ID No] |
|---|---|---|
| Samples 1 | Experiment 1 | TSNGRLPVK [986] |

d - Small cell lung cancer, iTRAQ

| Sample no. | Experiment no. | Tryptics identified [SEQ ID No] |
|---|---|---|
| Samples 1 | Experiment 1 | LVLGK [951] |

OGTA247

TABLE 89

| | | |
|---|---|---|
| Sample no. | Experiment no. | Tryptics identified [SEQ ID No] |

a - Non-small cell lung cancer, iTRAQ

| | | |
|---|---|---|
| Samples 1 | Experiment 1 | TNGTGRVR [984], WRPVDLAQVK [831] |
| Samples 1 | Experiment 2 | ERMFR [906] |

b - Small cell lung cancer, iTRAQ

| | | |
|---|---|---|
| Samples 1 | Experiment 1 | DATQITQGPR [109], DLQELGDSDK [137], DPELR |
| | | [893], EGPGEAIVR [189], NPVDVK [958], TNGTGRVR |
| | | [984], VGEEDDGEYR [762], VTAINK [1000], |
| | | YGPGEPSPVSETVVTPEAAPEK [1005] |
| Samples 1 | Experiment 2 | AQLLVVGSPGPVPR [82], DATQITQGPR [109], DPELR |
| | | [893], EPIDLR [217], NPVDVK [958], TNGTGRVR [984], |
| | | VGEEDDGEYR [762], VQAVNSQGK [998], VTAINK |
| | | [1000], YGPGEPSPVSETVVTPEAAPEK [1005] |

OGTA248

TABLE 90
Non-small cell lung cancer, iTRAQ

| | | Tryptics identified |
|---|---|---|
| Sample no. | Experiment no. | [SEQ ID No] |
| Samples 1 | Experiment 1 | LRTSAIR [945] |

OGTA249

TABLE 91

| | | |
|---|---|---|
| | | Tryptics identified |
| Sample no. | Experiment no. | [SEQ ID No] |

a - Kidney cancer, iTRAQ

| | | |
|---|---|---|
| Samples 1 | Experiment 1 | VTLPAGPDILR [1001] |

b - Non-small cell lung cancer, iTRAQ

| | | |
|---|---|---|
| Samples 1 | Experiment 1 | VTLPAGPDILR [1001] |
| Samples 2 | Experiment 1 | VTLPAGPDILR [1001] |
| Samples 2 | Experiment 2 | VTLPAGPDILR [1001] |
| Samples 2 | Experiment 3 | VTLPAGPDILR [1001] |

OGTA257

TABLE 92
a - Colorectal cancer, iTRAQ

| Samples batch no. | Experiment no. | Tryptics identified [SEQ ID No] |
|---|---|---|
| Samples 1 | Experiment 1 | QILDQTPVK [962] |

b - Liver cancer, iTRAQ

| Sample no. | Experiment no. | Tryptics identified [SEQ ID No] |
|---|---|---|
| Samples 1 | Experiment 1 | LRQGTLR [944] |

c - Non-small cell lung cancer, iTRAQ

| Sample no. | Experiment no. | Tryptics identified [SEQ ID No] |
|---|---|---|
| Samples 1 | Experiment 1 | ATGTAFRR [884], ELVSLK [902] |

d - Ovarian cancer, iTRAQ

| Sample no. | Experiment no. | Tryptics identified [SEQ ID No] |
|---|---|---|
| Samples 1 | Experiment 1 | LLIEHGADIR [936] |

OGTA271

TABLE 93
a - Colorectal cancer, iTRAQ

| Samples batch no. | Experiment no. | Tryptics identified [SEQ ID No] |
|---|---|---|
| Samples 1 | Experiment 1 | IKQLR [922], TAQSTPSAPPQK [672], |
| | | TATVVMMTRLEEK [979] |
| Samples 1 | Experiment 2 | VKCDQYWPAR [991] |

b - Kidney cancer, iTRAQ

| Sample no. | Experiment no. | Tryptics identified [SEQ ID No] |
|---|---|---|
| Samples 1 | Experiment 1 | TAQSTPSAPPQK [672] |
| Samples 1 | Experiment 2 | EPMDQKR [218] |

c - Liver cancer, iTRAQ

| Sample no. | Experiment no. | Tryptics identified [SEQ ID No] |
|---|---|---|
| Samples 1 | Experiment 1 | IKQLR [922] |

d - Non-small cell lung cancer, iTRAQ

| Sample no. | Experiment no. | Tryptics identified [SEQ ID No] |
|---|---|---|
| Samples 1 | Experiment 1 | NRAGLGEEFEKEIR [960], TAQSTPSAPPQK [672] |
| Samples 2 | Experiment 1 | DSLLAHSSDPVEMR [156], TMPVEQVFAK [708] |

For proteins of the invention the detected level obtained upon analyzing tissue from subjects having a relevant cancer relative to the detected level obtained upon analyzing tissue from subjects free from said cancers will depend upon the particular analytical protocol and detection technique that is used. Accordingly, the present invention contemplates that each laboratory will establish a reference range in subjects free from said cancers according to the analytical protocol and detection technique in use, as is conventional in the diagnostic art. For example, at least one control positive tissue sample from a subject known to have a relevant cancer or at least one control negative tissue sample from a subject known to be free from said cancer (and more preferably both positive and negative control samples) are included in each batch of test samples analysed.

Proteins of the invention can be used for detection, prognosis, diagnosis, or monitoring of a relevant cancer or for drug development. In one embodiment of the invention, tissue from a subject (e.g., a subject suspected of having a relevant cancer) is analysed by 1D electrophoresis or iTRAQ for detection of a protein according to the invention. An increased abundance of a protein according to the invention in the tissue from the subject relative to tissue from a subject or subjects free from said cancer (e.g., a control sample) or a previously determined reference range indicates the presence of a relevant cancer.

In particular, OGTA002 can be used for detection, prognosis, diagnosis, or monitoring of breast cancer, colorectal cancer, hepatocellular carcinoma, lung cancer, melanoma, ovarian cancer, pancreatic cancer and renal cell cancer.

In particular, OGTA009 can be used for detection, prognosis, diagnosis, or monitoring of breast cancer, colorectal cancer, lung cancer, ovarian cancer, pancreatic cancer, prostate cancer and renal cell cancer.

In particular, OGTA016 can be used for detection, prognosis, diagnosis, or monitoring of colorectal cancer and lung cancer.

In particular, OGTA028 can be used for detection, prognosis, diagnosis, or monitoring of colorectal cancer, hepatocellular carcinoma, lung cancer, ovarian cancer and renal cell cancer.

In particular, OGTA037 can be used for detection, prognosis, diagnosis, or monitoring of colorectal cancer, lung cancer, gastric cancer and ovarian cancer.

In particular, OGTA041 can be used for detection, prognosis, diagnosis, or monitoring of hepatocellular carcinoma, lung cancer and pancreatic cancer.

In particular, OGTA053 can be used for detection, prognosis, diagnosis, or monitoring of colorectal cancer, hepatocellular carcinoma, lung cancer, pancreatic cancer and renal cell cancer.

In particular, OGTA054 can be used for detection, prognosis, diagnosis, or monitoring of colorectal cancer, lung cancer and pancreatic cancer.

In particular, OGTA066 can be used for detection, prognosis, diagnosis, or monitoring of colorectal cancer, lung cancer, pancreatic cancer and renal cell cancer.

In particular, OGTA072 can be used for detection, prognosis, diagnosis, or monitoring of colorectal cancer.

In particular, OGTA074 can be used for detection, prognosis, diagnosis, or monitoring of colorectal cancer and lung cancer.

In particular, OGTA076 can be used for detection, prognosis, diagnosis, or monitoring of chronic lymphocytic leukaemia, colorectal cancer, hepatocellular carcinoma, lung cancer, ovarian cancer, pancreatic cancer and renal cell cancer.

In particular, OGTA085 can be used for detection, prognosis, diagnosis, or monitoring of colorectal cancer, lung cancer, melanoma and retinoblastoma.

In particular, OGTA087 can be used for detection, prognosis, diagnosis, or monitoring of B-cell non-Hodgkin's lymphoma, breast cancer, colorectal cancer, gastric cancer, lung cancer, lymphoid leukaemia and ovarian cancer.

In particular, OGTA088 can be used for detection, prognosis, diagnosis, or monitoring of breast cancer, colorectal cancer, gastric cancer, glioblastoma, hepatocellular carcinoma, lung cancer, lymphoid leukaemia, pancreatic cancer, renal cell cancer and retinoblastoma.

In particular, OGTA089 can be used for detection, prognosis, diagnosis, or monitoring of breast cancer, lung cancer, ovarian cancer and renal cell cancer.

In particular, OGTA091 can be used for detection, prognosis, diagnosis, or monitoring of breast cancer, cervical cancer, chronic lymphocytic leukaemia, colorectal cancer, gastric cancer, hepatocellular carcinoma, lung cancer, melanoma, osteosarcoma, ovarian cancer, pancreatic cancer, prostate cancer and renal cell cancer.

In particular, OGTA098 can be used for detection, prognosis, diagnosis, or monitoring of breast cancer, cervical cancer, colorectal cancer, hepatocellular carcinoma, lung cancer, osteosarcoma, ovarian cancer, pancreatic cancer, prostate cancer and renal cell cancer.

In particular, OGTA101 can be used for detection, prognosis, diagnosis, or monitoring of breast cancer, cervical cancer, colorectal cancer, hepatocellular carcinoma, lung cancer, osteosarcoma, ovarian cancer, pancreatic cancer and renal cell cancer.

In particular, OGTA104 can be used for detection, prognosis, diagnosis, or monitoring of breast cancer, colorectal cancer, hepatocellular carcinoma, lung cancer, ovarian cancer, pancreatic cancer and renal cell cancer.

In particular, OGTA106 can be used for detection, prognosis, diagnosis, or monitoring of cervical cancer, hepatocellular carcinoma, melanoma and pancreatic cancer.

In particular, OGTA112 can be used for detection, prognosis, diagnosis, or monitoring of breast cancer, cervical cancer, chronic lymphocytic leukaemia, hepatocellular carcinoma, pancreatic cancer and renal cell cancer.

In particular, OGTA113 can be used for detection, prognosis, diagnosis, or monitoring of colorectal cancer, lung cancer, ovarian cancer, pancreatic cancer and renal cell cancer.

In particular, OGTA119 can be used for detection, prognosis, diagnosis, or monitoring of B-cell non-Hodgkin's lymphoma, breast cancer, colorectal cancer, gastric cancer, hepatocellular carcinoma, lung cancer, lymphoid leukaemia, neuroblastoma, osteosarcoma, ovarian cancer, pancreatic cancer, prostate cancer and renal cell cancer.

In particular, OGTA124 can be used for detection, prognosis, diagnosis, or monitoring of colorectal cancer, hepatocellular carcinoma, lung cancer, ovarian cancer, pancreatic cancer and renal cell cancer.

In particular, OGTA126 can be used for detection, prognosis, diagnosis, or monitoring of breast cancer, colorectal cancer, hepatocellular carcinoma, lung cancer, melanoma, pancreatic cancer, prostate cancer and renal cell cancer.

In particular, OGTA156 can be used for detection, prognosis, diagnosis, or monitoring of acute T-cell leukaemia, B-cell non-Hodgkin's lymphoma, chronic lymphocytic leukaemia, colorectal cancer, hepatocellular carcinoma, lung cancer, ovarian cancer and renal cell cancer.

In particular, OGTA159 can be used for detection, prognosis, diagnosis, or monitoring of breast cancer, chronic lymphocytic leukaemia, colorectal cancer, hepatocellular carcinoma, lung cancer, melanoma, pancreatic cancer and renal cell cancer.

In particular, OGTA168 can be used for detection, prognosis, diagnosis, or monitoring of glioblastoma and melanoma.

In particular, OGTA169 can be used for detection, prognosis, diagnosis, or monitoring of breast cancer, chronic lymphocytic leukaemia, colorectal cancer, lung cancer and renal cell cancer.

In particular, OGTA174 can be used for detection, prognosis, diagnosis, or monitoring of acute T-cell leukaemia, chronic lymphocytic leukaemia and lung cancer.

In particular, OGTA176 can be used for detection, prognosis, diagnosis, or monitoring of B-cell non-Hodgkin's lymphoma, chronic lymphocytic leukaemia, colorectal cancer, lung cancer and renal cell cancer.

In particular, OGTA177 can be used for detection, prognosis, diagnosis, or monitoring of chronic lymphocytic leukaemia, colorectal cancer, lung cancer, ovarian cancer and renal cell cancer.

In particular, OGTA197 can be used for detection, prognosis, diagnosis, or monitoring of colorectal cancer, hepatocellular carcinoma, lung cancer, melanoma, osteosarcoma, ovarian cancer, pancreatic cancer, prostate cancer and renal cell cancer.

In particular, OGTA202 can be used for detection, prognosis, diagnosis, or monitoring of colorectal cancer and lung cancer.

In particular, OGTA203 can be used for detection, prognosis, diagnosis, or monitoring of lung cancer, ovarian cancer and renal cell cancer.

In particular, OGTA206 can be used for detection, prognosis, diagnosis, or monitoring of breast cancer, colorectal cancer, lung cancer, pancreatic cancer and prostate cancer.

In particular, OGTA213 can be used for detection, prognosis, diagnosis, or monitoring of colorectal cancer and lung cancer.

In particular, OGTA214 can be used for detection, prognosis, diagnosis, or monitoring of colorectal cancer and ovarian cancer.

In particular, OGTA216 can be used for detection, prognosis, diagnosis, or monitoring of breast cancer, colorectal cancer, hepatocellular carcinoma, lung cancer, ovarian cancer, pancreatic cancer and renal cell cancer.

In particular, OGTA222 can be used for detection, prognosis, diagnosis, or monitoring of B-cell non-Hodgkin's lymphoma, colorectal cancer, lung cancer and renal cell cancer.

In particular, OGTA236 can be used for detection, prognosis, diagnosis, or monitoring of colorectal cancer and pancreatic cancer.

In particular, OGTA237 can be used for detection, prognosis, diagnosis, or monitoring of B-cell non-Hodgkin's lymphoma, colorectal cancer, lung cancer, lymphoid leukaemia, prostate cancer and renal cell cancer.

In particular, OGTA247 can be used for detection, prognosis, diagnosis, or monitoring of hepatocellular carcinoma, lung cancer and melanoma.

In particular, OGTA248 can be used for detection, prognosis, diagnosis, or monitoring of lung cancer and renal cell cancer.

In particular, OGTA249 can be used for detection, prognosis, diagnosis, or monitoring of gastric cancer, lung cancer, ovarian cancer and renal cell cancer.

In particular, OGTA257 can be used for detection, prognosis, diagnosis, or monitoring of colorectal cancer, hepatocellular carcinoma, lung cancer, ovarian cancer and pancreatic cancer.

In particular, OGTA271 can be used for detection, prognosis, diagnosis, or monitoring of breast cancer, cervical cancer, colorectal cancer, hepatocellular carcinoma, lung cancer, osteosarcoma, pancreatic cancer and renal cell cancer.

In one or more aspects of the invention:

OGTA002 may, in particular, be characterized as an isoform having a MW substantially as recited (e.g. +/−10%, particularly +/−5% of the value) in column 1 of any of the rows of Tables 1a-1f

OGTA009 may, in particular, be characterized as an isoform having a MW substantially as recited (e.g. +/−10%, particularly +/−5% of the value) in column 1 of any of the rows of Tables 2a-2e

OGTA016 may, in particular, be characterized as an isoform having a MW substantially as recited (e.g. +/−10%, such as +/−5% of the value) in column 1 of any of the rows of Table 3

OGTA028 may, in particular, be characterized as an isoform having a MW substantially as recited (e.g. +/−10%, such as +/−5% of the value) in column 1 of any of the rows of Table 4

OGTA037 may, in particular, be characterized as an isoform having a MW substantially as recited (e.g. +/−10%, such as +/−5% of the value) in column 1 of any of the rows of Table 5

OGTA041 may, in particular, be characterized as an isoform having a MW substantially as recited (e.g. +/−10%, particularly +/−5% of the value) in column 1 of any of the rows of Tables 6a-6c

OGTA053 may, in particular, be characterized as an isoform having a MW substantially as recited (e.g. +/−10%, such as +/−5% of the value) in column 1 of any of the rows of Table 7

OGTA054 may, in particular, be characterized as an isoform having a MW substantially as recited (e.g. +/−10%, such as +/−5% of the value) in column 1 of any of the rows of Table 8

OGTA066 may, in particular, be characterized as an isoform having a MW substantially as recited (e.g. +/−10%, particularly +/−5% of the value) in column 1 of any of the rows of Tables 9a-9b

OGTA072 may, in particular, be characterized as an isoform having a MW substantially as recited (e.g. +/−10%, such as +/−5% of the value) in column 1 of any of the rows of Table 10

OGTA074 may, in particular, be characterized as an isoform having a MW substantially as recited (e.g. +/−10%, such as +/−5% of the value) in column 1 of any of the rows of Table 11

OGTA076 may, in particular, be characterized as an isoform having a MW substantially as recited (e.g. +/−10%, particularly +/−5% of the value) in column 1 of any of the rows of Tables 12a-12c

OGTA085 may, in particular, be characterized as an isoform having a MW substantially as recited (e.g. +/−10%, particularly +/−5% of the value) in column 1 of any of the rows of Tables 13a-13c

OGTA087 may, in particular, be characterized as an isoform having a MW substantially as recited (e.g. +/−10%, particularly +/−5% of the value) in column 1 of any of the rows of Tables 14a-14g

OGTA088 may, in particular, be characterized as an isoform having a MW substantially as recited (e.g. +/−10%, particularly +/−5% of the value) in column 1 of any of the rows of Tables 15a-15i

OGTA089 may, in particular, be characterized as an isoform having a MW substantially as recited (e.g. +/−10%, particularly +/−5% of the value) in column 1 of any of the rows of Tables 16a-16c

OGTA091 may, in particular, be characterized as an isoform having a MW substantially as recited (e.g. +/−10%, particularly +/−5% of the value) in column 1 of any of the rows of Tables 17a-171

OGTA098 may, in particular, be characterized as an isoform having a MW substantially as recited (e.g. +/−10%, particularly +/−5% of the value) in column 1 of any of the rows of Tables 18a-18h

OGTA101 may, in particular, be characterized as an isoform having a MW substantially as recited (e.g. +/−10%, particularly +/−5% of the value) in column 1 of any of the rows of Tables 19a-19g

OGTA104 may, in particular, be characterized as an isoform having a MW substantially as recited (e.g. +/−10%, particularly +/−5% of the value) in column 1 of any of the rows of Tables 20a-20d

OGTA106 may, in particular, be characterized as an isoform having a MW substantially as recited (e.g. +/−10%, particularly +/−5% of the value) in column 1 of any of the rows of Tables 21a-21c

OGTA112 may, in particular, be characterized as an isoform having a MW substantially as recited (e.g. +/−10%, particularly +/−5% of the value) in column 1 of any of the rows of Tables 22a-22f

OGTA113 may, in particular, be characterized as an isoform having a MW substantially as recited (e.g. +/−10%, particularly +/−5% of the value) in column 1 of any of the rows of Table

OGTA119 may, in particular, be characterized as an isoform having a MW substantially as recited (e.g. +/−10%, particularly +/−5% of the value) in column 1 of any of the rows of Tables 24a-241

OGTA124 may, in particular, be characterized as an isoform having a MW substantially as recited (e.g. +/−10%, particularly +/−5% of the value) in column 1 of any of the rows of Tables 25a-25e

OGTA126 may, in particular, be characterized as an isoform having a MW substantially as recited (e.g. +/−10%, particularly +/−5% of the value) in column 1 of any of the rows of Tables 26a-26g

OGTA156 may, in particular, be characterized as an isoform having a MW substantially as recited (e.g. +/−10%, particularly +/−5% of the value) in column 1 of any of the rows of Tables 27a-27d

OGTA159 may, in particular, be characterized as an isoform having a MW substantially as recited (e.g. +/−10%, particularly +/−5% of the value) in column 1 of any of the rows of Tables 28a-28f

OGTA168 may, in particular, be characterized as an isoform having a MW substantially as recited (e.g. +/−10%, particularly +/−5% of the value) in column 1 of any of the rows of Tables 29a-29b

OGTA169 may, in particular, be characterized as an isoform having a MW substantially as recited (e.g. +/−10%, particularly +/−5% of the value) in column 1 of any of the rows of Tables 30a-30b

OGTA174 may, in particular, be characterized as an isoform having a MW substantially as recited (e.g. +/−10%, particularly +/−5% of the value) in column 1 of any of the rows of Tables 31a-31b

OGTA176 may, in particular, be characterized as an isoform having a MW substantially as recited (e.g. +/−10%, particularly +/−5% of the value) in column 1 of any of the rows of Tables 32a-32b

OGTA177 may, in particular, be characterized as an isoform having a MW substantially as recited (e.g. +/−10%, such as +/−5% of the value) in column 1 of any of the rows of Table 33

OGTA197 may, in particular, be characterized as an isoform having a MW substantially as recited (e.g. +/−10%, particularly +/−5% of the value) in column 1 of any of the rows of Tables 34a-34i

OGTA202 may, in particular, be characterized as an isoform having a MW substantially as recited (e.g. +/−10%, particularly +/−5% of the value) in column 1 of any of the rows of Table

OGTA203 may, in particular, be characterized as an isoform having a MW substantially as recited (e.g. +/−10%, particularly +/−5% of the value) in column 1 of any of the rows of Tables 36a-36b

OGTA206 may, in particular, be characterized as an isoform having a MW substantially as recited (e.g. +/−10%, particularly +/−5% of the value) in column 1 of any of the rows of Tables 37a-37c

OGTA213 may, in particular, be characterized as an isoform having a MW substantially as recited (e.g. +/−10%, such as +/−5% of the value) in column 1 of any of the rows of Table 38

OGTA214 may, in particular, be characterized as an isoform having a MW substantially as recited (e.g. +/−10%, particularly +/−5% of the value) in column 1 of any of the rows of Table

OGTA216 may, in particular, be characterized as an isoform having a MW substantially as recited (e.g. +/−10%, particularly +/−5% of the value) in column 1 of any of the rows of Tables 40a-40b

OGTA222 may, in particular, be characterized as an isoform having a MW substantially as recited (e.g. +/−10%, particularly +/−5% of the value) in column 1 of any of the rows of Tables 41a-41b

OGTA236 may, in particular, be characterized as an isoform having a MW substantially as recited (e.g. +/−10%, such as +/−5% of the value) in column 1 of any of the rows of Table 42

OGTA237 may, in particular, be characterized as an isoform having a MW substantially as recited (e.g. +/−10%, particularly +/−5% of the value) in column 1 of any of the rows of Tables 43a-43c

OGTA247 may, in particular, be characterized as an isoform having a MW substantially as recited (e.g. +/−10%, particularly +/−5% of the value) in column 1 of any of the rows of Tables 44a-44b

OGTA248 may, in particular, be characterized as an isoform having a MW substantially as recited (e.g. +/−10%, such as +/−5% of the value) in column 1 of any of the rows of Table 45

OGTA249 may, in particular, be characterized as an isoform having a MW substantially as recited (e.g. +/−10%, particularly +/−5% of the value) in column 1 of any of the rows of Table 46.

OGTA257 may, in particular, be characterized as an isoform having a MW substantially as recited (e.g. +/−10%, such as +/−5% of the value) in column 1 of any of the rows of Table 47

OGTA271 may, in particular, be characterized as an isoform having a MW substantially as recited (e.g. +/−10%, particularly +/−5% of the value) in column 1 of any of the rows of Tables 48a-48h

In relation to fragments, immunogenic fragments or antigenic fragments of OGTA002:
for breast cancer applications: for example these comprise one or more of the sequences identified as tryptic sequences in the 3^{rd} column of Table 1a;
for colorectal cancer applications: for example these comprise one or more of the sequences identified as tryptic sequences in the 3^{rd} column of Table 1b and Table 49a;
for hepatocellular carcinoma applications: for example these comprise one or more of the sequences identified as tryptic sequences in the 3^{rd} column of Table 1c and Table 49c;
for lung cancer applications: for example these comprise one or more of the sequences identified as tryptic sequences in the 3^{rd} column of Table 49d;
for melanoma applications: for example these comprise one or more of the sequences identified as tryptic sequences in the 3^{rd} column of Table 1d;
for ovarian cancer applications: for example these comprise one or more of the sequences identified as tryptic sequences in the 3^{rd} column of Table 1e;
for pancreatic cancer applications: for example these comprise one or more of the sequences identified as tryptic sequences in the 3^{rd} column of Table 1f;
for renal cell cancer applications: for example these comprise one or more of the sequences identified as tryptic sequences in the 3^{rd} column of Table 49b.

In relation to fragments, immunogenic fragments or antigenic fragments of OGTA009:
for breast cancer applications: for example these comprise one or more of the sequences identified as tryptic sequences in the 3^{rd} column of Table 2a;
for colorectal cancer applications: for example these comprise one or more of the sequences identified as tryptic sequences in the 3^{rd} column of Table 2b and Table 50a;
for lung cancer applications: for example these comprise one or more of the sequences identified as tryptic sequences in the 3^{rd} column of Table 50b;
for ovarian cancer applications: for example these comprise one or more of the sequences identified as tryptic sequences in the 3^{rd} column of Table 50c;
for pancreatic cancer applications: for example these comprise one or more of the sequences identified as tryptic sequences in the 3^{rd} column of Table 2c;
for prostate cancer applications: for example these comprise one or more of the sequences identified as tryptic sequences in the 3^{rd} column of Table 2d;
for renal cell cancer applications: for example these comprise one or more of the sequences identified as tryptic sequences in the 3^{rd} column of Table 2e;

In relation to fragments, immunogenic fragments or antigenic fragments of OGTA016:
for colorectal cancer applications: for example these comprise one or more of the sequences identified as tryptic sequences in the 3^{rd} column of Table 3 and Table 51a;
for lung cancer applications: for example these comprise one or more of the sequences identified as tryptic sequences in the 3^{rd} column of Table 51b.

In relation to fragments, immunogenic fragments or antigenic fragments of OGTA028:
for colorectal cancer applications: for example these comprise one or more of the sequences identified as tryptic sequences in the 3^{rd} column of Table 52a;
for hepatocellular carcinoma applications: for example these comprise one or more of the sequences identified as tryptic sequences in the 3^{rd} column of Table 52c; for lung cancer applications: for example these comprise one or more of the sequences identified as tryptic sequences in the 3^{rd} column of Table 52d and Table 52e;
for ovarian cancer applications: for example these comprise one or more of the sequences identified as tryptic sequences in the 3^{rd} column of Table 4 and Table 52f,
for renal cell cancer applications: for example these comprise one or more of the sequences identified as tryptic sequences in the 3^{rd} column of Table 52b.

In relation to fragments, immunogenic fragments or antigenic fragments of OGTA037:
for colorectal cancer applications: for example these comprise one or more of the sequences identified as tryptic sequences in the 3^{rd} column of Table 53a;
for lung cancer applications: for example these comprise one or more of the sequences identified as tryptic sequences in the 3^{rd} column of Table 53b;
for gastric cancer applications: for example these comprise one or more of the sequences identified as tryptic sequences in the 3^{rd} column of Table 5;
for ovarian cancer applications: for example these comprise one or more of the sequences identified as tryptic sequences in the 3^{rd} column of Table 53c.

In relation to fragments, immunogenic fragments or antigenic fragments of OGTA041:
for hepatocellular carcinoma applications: for example these comprise one or more of the sequences identified as tryptic sequences in the 3^{rd} column of Table 6a;
for lung cancer applications: for example these comprise one or more of the sequences identified as tryptic sequences in the 3^{rd} column of Table 6b and Table 54;
for pancreatic cancer applications: for example these comprise one or more of the sequences identified as tryptic sequences in the 3^{rd} column of Table 6c.

In relation to fragments, immunogenic fragments or antigenic fragments of OGTA053:
for colorectal cancer applications: for example these comprise one or more of the sequences identified as tryptic sequences in the 3^{rd} column of Table 55a;
for hepatocellular carcinoma applications: for example these comprise one or more of the sequences identified as tryptic sequences in the 3^{rd} column of Table 55c;
for lung cancer applications: for example these comprise one or more of the sequences identified as tryptic sequences in the 3^{rd} column of Table 55d and Table 55e;
for pancreatic cancer applications: for example these comprise one or more of the sequences identified as tryptic sequences in the 3^{rd} column of Table 7;
for renal cell cancer applications: for example these comprise one or more of the sequences identified as tryptic sequences in the 3^{rd} column of Table 55b.

In relation to fragments, immunogenic fragments or antigenic fragments of OGTA054:
for colorectal cancer applications: for example these comprise one or more of the sequences identified as tryptic sequences in the 3^{rd} column of Table 56a;
for lung cancer applications: for example these comprise one or more of the sequences identified as tryptic sequences in the 3^{rd} column of Table 56b;
for pancreatic cancer applications: for example these comprise one or more of the sequences identified as tryptic sequences in the 3^{rd} column of Table 8.

In relation to fragments, immunogenic fragments or antigenic fragments of OGTA066:
for colorectal cancer applications: for example these comprise one or more of the sequences identified as tryptic sequences in the 3^{rd} column of Table 9a and Table 57a;
for lung cancer applications: for example these comprise one or more of the sequences identified as tryptic sequences in the 3^{rd} column of Table 57c;
for pancreatic cancer applications: for example these comprise one or more of the sequences identified as tryptic sequences in the 3^{rd} column of Table 9b;
for renal cell cancer applications: for example these comprise one or more of the sequences identified as tryptic sequences in the 3^{rd} column of Table 57b.

In relation to fragments, immunogenic fragments or antigenic fragments of OGTA072:
for colorectal cancer applications: for example these comprise one or more of the sequences identified as tryptic sequences in the 3^{rd} column of Table 10.

In relation to fragments, immunogenic fragments or antigenic fragments of OGTA074:
for colorectal cancer applications: for example these comprise one or more of the sequences identified as tryptic sequences in the 3^{rd} column of Table 11;
for lung cancer applications: for example these comprise one or more of the sequences identified as tryptic sequences in the 3^{rd} column of Table 58.

In relation to fragments, immunogenic fragments or antigenic fragments of OGTA076:
for chronic lymphocytic leukaemia applications: for example these comprise one or more of the sequences identified as tryptic sequences in the 3^{rd} column of Table 12a;
for colorectal cancer applications: for example these comprise one or more of the sequences identified as tryptic sequences in the 3^{rd} column of Table 12b and Table 59a;
for hepatocellular carcinoma applications: for example these comprise one or more of the sequences identified as tryptic sequences in the 3^{rd} column of Table 59c;
for lung cancer applications: for example these comprise one or more of the sequences identified as tryptic sequences in the 3^{rd} column of Table 59d and Table 59e;
for ovarian cancer applications: for example these comprise one or more of the sequences identified as tryptic sequences in the 3^{rd} column of Table 59f;
for pancreatic cancer applications: for example these comprise one or more of the sequences identified as tryptic sequences in the 3^{rd} column of Table 12c;
for renal cell cancer applications: for example these comprise one or more of the sequences identified as tryptic sequences in the 3^{rd} column of Table 59b.

In relation to fragments, immunogenic fragments or antigenic fragments of OGTA085:
for colorectal cancer applications: for example these comprise one or more of the sequences identified as tryptic sequences in the 3^{rd} column of Table 60a;
for lung cancer applications: for example these comprise one or more of the sequences identified as tryptic sequences in the 3^{rd} column of Table 13a and Table 60b;
for melanoma applications: for example these comprise one or more of the sequences identified as tryptic sequences in the 3^{rd} column of Table 13b;
for retinoblastoma applications: for example these comprise one or more of the sequences identified as tryptic sequences in the 3^{rd} column of Table 13c.

In relation to fragments, immunogenic fragments or antigenic fragments of OGTA087:
for B-cell non-Hodgkin's lymphoma applications: for example these comprise one or more of the sequences identified as tryptic sequences in the 3^{rd} column of Table 14a;
for breast cancer applications: for example these comprise one or more of the sequences identified as tryptic sequences in the 3^{rd} column of Table 14b;
for colorectal cancer applications: for example these comprise one or more of the sequences identified as tryptic sequences in the 3^{rd} column of Table 14c;
for gastric cancer applications: for example these comprise one or more of the sequences identified as tryptic sequences in the 3^{rd} column of Table 14d;
for lung cancer applications: for example these comprise one or more of the sequences identified as tryptic sequences in the 3^{rd} column of Table 14e;
for lymphoid leukaemia applications: for example these comprise one or more of the sequences identified as tryptic sequences in the 3^{rd} column of Table 14f; for ovarian cancer applications: for example these comprise one or more of the sequences identified as tryptic sequences in the 3^{rd} column of Table 14g.

In relation to fragments, immunogenic fragments or antigenic fragments of OGTA088:
for breast cancer applications: for example these comprise one or more of the sequences identified as tryptic sequences in the 3^{rd} column of Table 15a;
for colorectal cancer applications: for example these comprise one or more of the sequences identified as tryptic sequences in the 3^{rd} column of Table 61a;
for gastric cancer applications: for example these comprise one or more of the sequences identified as tryptic sequences in the 3^{rd} column of Table 15b;
for glioblastoma applications: for example these comprise one or more of the sequences identified as tryptic sequences in the 3^{rd} column of Table 15c;
for hepatocellular carcinoma applications: for example these comprise one or more of the sequences identified as tryptic sequences in the 3^{rd} column of Table 15d;
for lung cancer applications: for example these comprise one or more of the sequences identified as tryptic sequences in the 3^{rd} column of Table 15e and Table 61b;
for lymphoid leukaemia applications: for example these comprise one or more of the sequences identified as tryptic sequences in the 3^{rd} column of Table 15f;
for pancreatic cancer applications: for example these comprise one or more of the sequences identified as tryptic sequences in the 3^{rd} column of Table 15g;
for renal cell cancer applications: for example these comprise one or more of the sequences identified as tryptic sequences in the 3^{rd} column of Table 15h;
for retinoblastoma applications: for example these comprise one or more of the sequences identified as tryptic sequences in the 3^{rd} column of Table 15i.

In relation to fragments, immunogenic fragments or antigenic fragments of OGTA089:
for breast cancer applications: for example these comprise one or more of the sequences identified as tryptic sequences in the 3^{rd} column of Table 16a;
for lung cancer applications: for example these comprise one or more of the sequences identified as tryptic sequences in the 3^{rd} column of Table 16b and Table 62b;
for ovarian cancer applications: for example these comprise one or more of the sequences identified as tryptic sequences in the 3^{rd} column of Table 16c and Table 62c;
for renal cell cancer applications: for example these comprise one or more of the sequences identified as tryptic sequences in the 3^{rd} column of Table 62a.

In relation to fragments, immunogenic fragments or antigenic fragments of OGTA091:
for breast cancer applications: for example these comprise one or more of the sequences identified as tryptic sequences in the 3^{rd} column of Table 17a;
for cervical cancer applications: for example these comprise one or more of the sequences identified as tryptic sequences in the 3^{rd} column of Table 17b;
for chronic lymphocytic leukaemia applications: for example these comprise one or more of the sequences identified as tryptic sequences in the 3^{rd} column of Table 17c;
for colorectal cancer applications: for example these comprise one or more of the sequences identified as tryptic sequences in the 3^{rd} column of Table 63a;
for gastric cancer applications: for example these comprise one or more of the sequences identified as tryptic sequences in the 3^{rd} column of Table 17d;
for hepatocellular carcinoma applications: for example these comprise one or more of the sequences identified as tryptic sequences in the 3^{rd} column of Table 17e and Table 63c;
for lung cancer applications: for example these comprise one or more of the sequences identified as tryptic sequences in the 3^{rd} column of Table 17f, Table 63d and Table 63e;
for melanoma applications: for example these comprise one or more of the sequences identified as tryptic sequences in the 3^{rd} column of Table 17g;
for osteosarcoma applications: for example these comprise one or more of the sequences identified as tryptic sequences in the 3^{rd} column of Table 17h;
for ovarian cancer applications: for example these comprise one or more of the sequences identified as tryptic sequences in the 3^{rd} column of Table 17i and Table 63f;
for pancreatic cancer applications: for example these comprise one or more of the sequences identified as tryptic sequences in the 3^{rd} column of Table 17j;
for prostate cancer applications: for example these comprise one or more of the sequences identified as tryptic sequences in the 3^{rd} column of Table 17k;
for renal cell cancer applications: for example these comprise one or more of the sequences identified as tryptic sequences in the 3^{rd} column of Table 171 and Table 63b.

In relation to fragments, immunogenic fragments or antigenic fragments of OGTA098:
for breast cancer applications: for example these comprise one or more of the sequences identified as tryptic sequences in the 3^{rd} column of Table 18a;
for cervical cancer applications: for example these comprise one or more of the sequences identified as tryptic sequences in the 3^{rd} column of Table 18b;
for colorectal cancer applications: for example these comprise one or more of the sequences identified as tryptic sequences in the 3^{rd} column of Table 64a;
for hepatocellular carcinoma applications: for example these comprise one or more of the sequences identified as tryptic sequences in the 3^{rd} column of Table 18c;
for lung cancer applications: for example these comprise one or more of the sequences identified as tryptic sequences in the 3^{rd} column of Table 18d, Table 64c and Table 64d;
for osteosarcoma applications: for example these comprise one or more of the sequences identified as tryptic sequences in the 3^{rd} column of Table 18e;
for ovarian cancer applications: for example these comprise one or more of the sequences identified as tryptic sequences in the 3^{rd} column of Table 64e;
for pancreatic cancer applications: for example these comprise one or more of the sequences identified as tryptic sequences in the 3^{rd} column of Table 18f;
for prostate cancer applications: for example these comprise one or more of the sequences identified as tryptic sequences in the 3^{rd} column of Table 18g;
for renal cell cancer applications: for example these comprise one or more of the sequences identified as tryptic sequences in the 3^{rd} column of Table 18h and Table 64b.

In relation to fragments, immunogenic fragments or antigenic fragments of OGTA101:
for breast cancer applications: for example these comprise one or more of the sequences identified as tryptic sequences in the 3^{rd} column of Table 19a;
for cervical cancer applications: for example these comprise one or more of the sequences identified as tryptic sequences in the 3^{rd} column of Table 19b;
for colorectal cancer applications: for example these comprise one or more of the sequences identified as tryptic sequences in the 3^{rd} column of Table 19c and Table 65a;
for hepatocellular carcinoma applications: for example these comprise one or more of the sequences identified as tryptic sequences in the 3^{rd} column of Table 19d and Table 65c;
for lung cancer applications: for example these comprise one or more of the sequences identified as tryptic sequences in the 3^{rd} column of Table 19e and Table 65d;
for osteosarcoma applications: for example these comprise one or more of the sequences identified as tryptic sequences in the 3^{rd} column of Table 19f;
for ovarian cancer applications: for example these comprise one or more of the sequences identified as tryptic sequences in the 3^{rd} column of Table 65e;
for pancreatic cancer applications: for example these comprise one or more of the sequences identified as tryptic sequences in the 3^{rd} column of Table 19g;
for renal cell cancer applications: for example these comprise one or more of the sequences identified as tryptic sequences in the 3^{rd} column of Table 65b.

In relation to fragments, immunogenic fragments or antigenic fragments of OGTA104:
for breast cancer applications: for example these comprise one or more of the sequences identified as tryptic sequences in the 3^{rd} column of Table 20a;
for colorectal cancer applications: for example these comprise one or more of the sequences identified as tryptic sequences in the 3^{rd} column of Table 66a;
for hepatocellular carcinoma applications: for example these comprise one or more of the sequences identified as tryptic sequences in the 3^{rd} column of Table 20b and Table 66c;
for lung cancer applications: for example these comprise one or more of the sequences identified as tryptic sequences in the 3^{rd} column of Table 66d;
for ovarian cancer applications: for example these comprise one or more of the sequences identified as tryptic sequences in the 3^{rd} column of Table 20c and Table 66e;
for pancreatic cancer applications: for example these comprise one or more of the sequences identified as tryptic sequences in the 3^{rd} column of Table 20d;
for renal cell cancer applications: for example these comprise one or more of the sequences identified as tryptic sequences in the 3^{rd} column of Table 66b.

In relation to fragments, immunogenic fragments or antigenic fragments of OGTA106:
for cervical cancer applications: for example these comprise one or more of the sequences identified as tryptic sequences in the 3^{rd} column of Table 21a;
for hepatocellular carcinoma applications: for example these comprise one or more of the sequences identified as tryptic sequences in the 3^{rd} column of Table 67;
for melanoma applications: for example these comprise one or more of the sequences identified as tryptic sequences in the 3^{rd} column of Table 21b;
for pancreatic cancer applications: for example these comprise one or more of the sequences identified as tryptic sequences in the 3^{rd} column of Table 21c.

In relation to fragments, immunogenic fragments or antigenic fragments of OGTA112:
for breast cancer applications: for example these comprise one or more of the sequences identified as tryptic sequences in the 3^{rd} column of Table 22a;
for cervical cancer applications: for example these comprise one or more of the sequences identified as tryptic sequences in the 3^{rd} column of Table 22b;
for chronic lymphocytic leukaemia applications: for example these comprise one or more of the sequences identified as tryptic sequences in the 3^{rd} column of Table 22c;
for hepatocellular carcinoma applications: for example these comprise one or more of the sequences identified as tryptic sequences in the 3^{rd} column of Table 22d;
for pancreatic cancer applications: for example these comprise one or more of the sequences identified as tryptic sequences in the 3^{rd} column of Table 22e;
for renal cell cancer applications: for example these comprise one or more of the sequences identified as tryptic sequences in the 3^{rd} column of Table 22f and Table 68.

In relation to fragments, immunogenic fragments or antigenic fragments of OGTA113:
for colorectal cancer applications: for example these comprise one or more of the sequences identified as tryptic sequences in the 3^{rd} column of Table 69a;
for lung cancer applications: for example these comprise one or more of the sequences identified as tryptic sequences in the 3^{rd} column of Table 69c;
for ovarian cancer applications: for example these comprise one or more of the sequences identified as tryptic sequences in the 3^{rd} column of Table 69d;
for pancreatic cancer applications: for example these comprise one or more of the sequences identified as tryptic sequences in the 3^{rd} column of Table 23;
for renal cell cancer applications: for example these comprise one or more of the sequences identified as tryptic sequences in the 3^{rd} column of Table 69b.

In relation to fragments, immunogenic fragments or antigenic fragments of OGTA119:
for B-cell non-Hodgkin's lymphoma applications: for example these comprise one or more of the sequences identified as tryptic sequences in the 3^{rd} column of Table 24a;
for breast cancer applications: for example these comprise one or more of the sequences identified as tryptic sequences in the 3^{rd} column of Table 24b;
for colorectal cancer applications: for example these comprise one or more of the sequences identified as tryptic sequences in the 3^{rd} column of Table 24c and Table 70a;
for gastric cancer applications: for example these comprise one or more of the sequences identified as tryptic sequences in the 3^{rd} column of Table 24d;
for hepatocellular carcinoma applications: for example these comprise one or more of the sequences identified as tryptic sequences in the 3^{rd} column of Table 24e;
for lung cancer applications: for example these comprise one or more of the sequences identified as tryptic sequences in the 3^{rd} column of Table 24f and Table 70b;
for lymphoid leukaemia applications: for example these comprise one or more of the sequences identified as tryptic sequences in the 3^{rd} column of Table 24g;
for neuroblastoma applications: for example these comprise one or more of the sequences identified as tryptic sequences in the 3^{rd} column of Table 24h;
for osteosarcoma applications: for example these comprise one or more of the sequences identified as tryptic sequences in the 3^{rd} column of Table 24i;
for ovarian cancer applications: for example these comprise one or more of the sequences identified as tryptic sequences in the 3^{rd} column of Table 70c;
for pancreatic cancer applications: for example these comprise one or more of the sequences identified as tryptic sequences in the 3^{rd} column of Table 24j;
for prostate cancer applications: for example these comprise one or more of the sequences identified as tryptic sequences in the 3^{rd} column of Table 24k;
for renal cell cancer applications: for example these comprise one or more of the sequences identified as tryptic sequences in the 3^{rd} column of Table 241.

In relation to fragments, immunogenic fragments or antigenic fragments of OGTA124:
for colorectal cancer applications: for example these comprise one or more of the sequences identified as tryptic sequences in the 3^{rd} column of Table 71a;
for hepatocellular carcinoma applications: for example these comprise one or more of the sequences identified as tryptic sequences in the 3^{rd} column of Table 25a and Table 71c;
for lung cancer applications: for example these comprise one or more of the sequences identified as tryptic sequences in the 3^{rd} column of Table 25b and Table 71d;
for ovarian cancer applications: for example these comprise one or more of the sequences identified as tryptic sequences in the 3^{rd} column of Table 25c and Table 71e;
for pancreatic cancer applications: for example these comprise one or more of the sequences identified as tryptic sequences in the 3^{rd} column of Table 25d;
for renal cell cancer applications: for example these comprise one or more of the sequences identified as tryptic sequences in the 3^{rd} column of Table 25e and Table 71b.

In relation to fragments, immunogenic fragments or antigenic fragments of OGTA126:
for breast cancer applications: for example these comprise one or more of the sequences identified as tryptic sequences in the 3^{rd} column of Table 26a;
for colorectal cancer applications: for example these comprise one or more of the sequences identified as tryptic sequences in the 3^{rd} column of Table 26b;
for hepatocellular carcinoma applications: for example these comprise one or more of the sequences identified as tryptic sequences in the 3^{rd} column of Table 26c;
for lung cancer applications: for example these comprise one or more of the sequences identified as tryptic sequences in the 3^{rd} column of Table 72;
for melanoma applications: for example these comprise one or more of the sequences identified as tryptic sequences in the 3^{rd} column of Table 26d;
for pancreatic cancer applications: for example these comprise one or more of the sequences identified as tryptic sequences in the 3^{rd} column of Table 26e;
for prostate cancer applications: for example these comprise one or more of the sequences identified as tryptic sequences in the 3^{rd} column of Table 26f;
for renal cell cancer applications: for example these comprise one or more of the sequences identified as tryptic sequences in the 3^{rd} column of Table 26g.

In relation to fragments, immunogenic fragments or antigenic fragments of OGTA156:
for acute T-cell leukaemia applications: for example these comprise one or more of the sequences identified as tryptic sequences in the 3^{rd} column of Table 27a;
for B-cell non-Hodgkin's lymphoma applications: for example these comprise one or more of the sequences identified as tryptic sequences in the 3^{rd} column of Table 27b;
for chronic lymphocytic leukaemia applications: for example these comprise one or more of the sequences identified as tryptic sequences in the 3^{rd} column of Table 27c;
for colorectal cancer applications: for example these comprise one or more of the sequences identified as tryptic sequences in the 3^{rd} column of Table 73a;
for hepatocellular carcinoma applications: for example these comprise one or more of the sequences identified as tryptic sequences in the 3^{rd} column of Table 27d;
for lung cancer applications: for example these comprise one or more of the sequences identified as tryptic sequences in the 3^{rd} column of Table 73c;
for ovarian cancer applications: for example these comprise one or more of the sequences identified as tryptic sequences in the 3^{rd} column of Table 73d;
for renal cell cancer applications: for example these comprise one or more of the sequences identified as tryptic sequences in the 3^{rd} column of Table 73b.

In relation to fragments, immunogenic fragments or antigenic fragments of OGTA159:
for breast cancer applications: for example these comprise one or more of the sequences identified as tryptic sequences in the 3^{rd} column of Table 28a;
for chronic lymphocytic leukaemia applications: for example these comprise one or more of the sequences identified as tryptic sequences in the 3^{rd} column of Table 28b;
for colorectal cancer applications: for example these comprise one or more of the sequences identified as tryptic sequences in the 3^{rd} column of Table 28c;
for hepatocellular carcinoma applications: for example these comprise one or more of the sequences identified as tryptic sequences in the 3^{rd} column of Table 28d;
for lung cancer applications: for example these comprise one or more of the sequences identified as tryptic sequences in the 3^{rd} column of Table 74b;
for melanoma applications: for example these comprise one or more of the sequences identified as tryptic sequences in the 3^{rd} column of Table 28e;
for pancreatic cancer applications: for example these comprise one or more of the sequences identified as tryptic sequences in the 3^{rd} column of Table 28f;
for renal cell cancer applications: for example these comprise one or more of the sequences identified as tryptic sequences in the 3^{rd} column of Table 74a.

In relation to fragments, immunogenic fragments or antigenic fragments of OGTA168:
for glioblastoma applications: for example these comprise one or more of the sequences identified as tryptic sequences in the 3^{rd} column of Table 29a;
for melanoma applications: for example these comprise one or more of the sequences identified as tryptic sequences in the 3^{rd} column of Table 29b.

In relation to fragments, immunogenic fragments or antigenic fragments of OGTA169:
for breast cancer applications: for example these comprise one or more of the sequences identified as tryptic sequences in the 3^{rd} column of Table 30a;
for chronic lymphocytic leukaemia applications: for example these comprise one or more of the sequences identified as tryptic sequences in the 3^{rd} column of Table 30b;
for colorectal cancer applications: for example these comprise one or more of the sequences identified as tryptic sequences in the 3^{rd} column of Table 75a;
for lung cancer applications: for example these comprise one or more of the sequences identified as tryptic sequences in the 3^{rd} column of Table 75c;
for renal cell cancer applications: for example these comprise one or more of the sequences identified as tryptic sequences in the 3^{rd} column of Table 75b.

In relation to fragments, immunogenic fragments or antigenic fragments of OGTA174:
for acute T-cell leukaemia applications: for example these comprise one or more of the sequences identified as tryptic sequences in the 3^{rd} column of Table 31a;
for chronic lymphocytic leukaemia applications: for example these comprise one or more of the sequences identified as tryptic sequences in the 3^{rd} column of Table 31b;
for lung cancer applications: for example these comprise one or more of the sequences identified as tryptic sequences in the 3^{rd} column of Table 76.

In relation to fragments, immunogenic fragments or antigenic fragments of OGTA176:
for B-cell non-Hodgkin's lymphoma applications: for example these comprise one or more of the sequences identified as tryptic sequences in the 3^{rd} column of Table 32a;
for chronic lymphocytic leukaemia applications: for example these comprise one or more of the sequences identified as tryptic sequences in the 3^{rd} column of Table 32b;
for colorectal cancer applications: for example these comprise one or more of the sequences identified as tryptic sequences in the 3^{rd} column of Table 77a;
for lung cancer applications: for example these comprise one or more of the sequences identified as tryptic sequences in the 3^{rd} column of Table 77c and Table 77d;
for renal cell cancer applications: for example these comprise one or more of the sequences identified as tryptic sequences in the 3^{rd} column of Table 77b.

In relation to fragments, immunogenic fragments or antigenic fragments of OGTA177:
for chronic lymphocytic leukaemia applications: for example these comprise one or more of the sequences identified as tryptic sequences in the 3^{rd} column of Table 33;
for colorectal cancer applications: for example these comprise one or more of the sequences identified as tryptic sequences in the 3^{rd} column of Table 78a;
for lung cancer applications: for example these comprise one or more of the sequences identified as tryptic sequences in the 3^{rd} column of Table 78c and Table 78d;
for ovarian cancer applications: for example these comprise one or more of the sequences identified as tryptic sequences in the 3^{rd} column of Table 78e;
for renal cell cancer applications: for example these comprise one or more of the sequences identified as tryptic sequences in the 3^{rd} column of Table 78b;

In relation to fragments, immunogenic fragments or antigenic fragments of OGTA197:
for colorectal cancer applications: for example these comprise one or more of the sequences identified as tryptic sequences in the 3^{rd} column of Table 34a and Table 79a;
for hepatocellular carcinoma applications: for example these comprise one or more of the sequences identified as tryptic sequences in the 3^{rd} column of Table 34b and Table 79c;
for lung cancer applications: for example these comprise one or more of the sequences identified as tryptic sequences in the 3^{rd} column of Table 34c, Table 79d and Table 79e;
for melanoma applications: for example these comprise one or more of the sequences identified as tryptic sequences in the 3^{rd} column of Table 34d;
for osteosarcoma applications: for example these comprise one or more of the sequences identified as tryptic sequences in the 3^{rd} column of Table 34e;
for ovarian cancer applications: for example these comprise one or more of the sequences identified as tryptic sequences in the 3^{rd} column of Table 34f;
for pancreatic cancer applications: for example these comprise one or more of the sequences identified as tryptic sequences in the 3^{rd} column of Table 34g;
for prostate cancer applications: for example these comprise one or more of the sequences identified as tryptic sequences in the 3^{rd} column of Table 34h;
for renal cell cancer applications: for example these comprise one or more of the sequences identified as tryptic sequences in the 3^{rd} column of Table 34i and Table 79b.

In relation to fragments, immunogenic fragments or antigenic fragments of OGTA0202:
for colorectal cancer applications: for example these comprise one or more of the sequences identified as tryptic sequences in the 3^{rd} column of Table 35 and Table 80a;
for lung cancer applications: for example these comprise one or more of the sequences identified as tryptic sequences in the 3^{rd} column of Table 80b.

In relation to fragments, immunogenic fragments or antigenic fragments of OGTA203:
for lung cancer applications: for example these comprise one or more of the sequences identified as tryptic sequences in the 3^{rd} column of Table 81b;
for ovarian cancer applications: for example these comprise one or more of the sequences identified as tryptic sequences in the 3^{rd} column of Table 36a;
for renal cell cancer applications: for example these comprise one or more of the sequences identified as tryptic sequences in the 3^{rd} column of Table 36b and Table 81a.

In relation to fragments, immunogenic fragments or antigenic fragments of OGTA206:
for breast cancer applications: for example these comprise one or more of the sequences identified as tryptic sequences in the 3^{rd} column of Table 37a;
for colorectal cancer applications: for example these comprise one or more of the sequences identified as tryptic sequences in the 3^{rd} column of Table 82a;
for lung cancer applications: for example these comprise one or more of the sequences identified as tryptic sequences in the 3^{rd} column of Table 82b;
for pancreatic cancer applications: for example these comprise one or more of the sequences identified as tryptic sequences in the 3^{rd} column of Table 37b;
for prostate cancer applications: for example these comprise one or more of the sequences identified as tryptic sequences in the 3^{rd} column of Table 37c.

In relation to fragments, immunogenic fragments or antigenic fragments of OGTA213:
for colorectal cancer applications: for example these comprise one or more of the sequences identified as tryptic sequences in the 3^{rd} column of Table 83a;
for lung cancer applications: for example these comprise one or more of the sequences identified as tryptic sequences in the 3^{rd} column of Table 38 and Table 83b.

In relation to fragments, immunogenic fragments or antigenic fragments of OGTA214:
for colorectal cancer applications: for example these comprise one or more of the sequences identified as tryptic sequences in the 3^{rd} column of Table 84;
for ovarian cancer applications: for example these comprise one or more of the sequences identified as tryptic sequences in the 3^{rd} column of Table 39.

In relation to fragments, immunogenic fragments or antigenic fragments of OGTA216:
for breast cancer applications: for example these comprise one or more of the sequences identified as tryptic sequences in the 3^{rd} column of Table 40a;
for colorectal cancer applications: for example these comprise one or more of the sequences identified as tryptic sequences in the 3^{rd} column of Table 40b and Table 85a;
for hepatocellular carcinoma applications: for example these comprise one or more of the sequences identified as tryptic sequences in the 3^{rd} column of Table 85c;
for lung cancer applications: for example these comprise one or more of the sequences identified as tryptic sequences in the 3^{rd} column of Table 85d and Table 85e;
for ovarian cancer applications: for example these comprise one or more of the sequences identified as tryptic sequences in the 3^{rd} column of Table 85f;
for pancreatic cancer applications: for example these comprise one or more of the sequences identified as tryptic sequences in the 3^{rd} column of Table 40c;
for renal cell cancer applications: for example these comprise one or more of the sequences identified as tryptic sequences in the 3^{rd} column of Table 85b.

In relation to fragments, immunogenic fragments or antigenic fragments of OGTA222:
for B-cell non-Hodgkin's lymphoma applications: for example these comprise one or more of the sequences identified as tryptic sequences in the 3^{rd} column of Table 41a;
for colorectal cancer applications: for example these comprise one or more of the sequences identified as tryptic sequences in the 3^{rd} column of Table 41b and Table 86a;
for lung cancer applications: for example these comprise one or more of the sequences identified as tryptic sequences in the 3^{rd} column of Table 86c;
for renal cell applications: for example these comprise one or more of the sequences identified as tryptic sequences in the 3^{rd} column of Table 86b.

In relation to fragments, immunogenic fragments or antigenic fragments of OGTA236:
for colorectal cancer applications: for example these comprise one or more of the sequences identified as tryptic sequences in the 3^{rd} column of Table 87;
for pancreatic cancer applications: for example these comprise one or more of the sequences identified as tryptic sequences in the 3^{rd} column of Table 42.

In relation to fragments, immunogenic fragments or antigenic fragments of OGTA237:
for B-cell non-Hodgkin's lymphoma applications: for example these comprise one or more of the sequences identified as tryptic sequences in the 3^{rd} column of Table 43a;
for colorectal cancer applications: for example these comprise one or more of the sequences identified as tryptic sequences in the 3^{rd} column of Table 88a;
for lung cancer applications: for example these comprise one or more of the sequences identified as tryptic sequences in the 3^{rd} column of Table 88c and table 88d;
for lymphoid leukaemia applications: for example these comprise one or more of the sequences identified as tryptic sequences in the 3^{rd} column of Table 43b;
for prostate cancer applications: for example these comprise one or more of the sequences identified as tryptic sequences in the 3^{rd} column of Table 43c;
for renal cell cancer applications: for example these comprise one or more of the sequences identified as tryptic sequences in the 3^{rd} column of Table 88b.

In relation to fragments, immunogenic fragments or antigenic fragments of OGTA247:
for hepatocellular carcinoma applications: for example these comprise one or more of the sequences identified as tryptic sequences in the 3^{rd} column of Table 44a;
for lung cancer applications: for example these comprise one or more of the sequences identified as tryptic sequences in the 3^{rd} column of Table 89a and Table 89b;
for melanoma applications: for example these comprise one or more of the sequences identified as tryptic sequences in the 3^{rd} column of Table 44b.

In relation to fragments, immunogenic fragments or antigenic fragments of OGTA248:
for lung cancer applications: for example these comprise one or more of the sequences identified as tryptic sequences in the 3^{rd} column of Table 90.
for renal cell cancer applications: for example these comprise one or more of the sequences identified as tryptic sequences in the 3^{rd} column of Table 45.

In relation to fragments, immunogenic fragments or antigenic fragments of OGTA249:
for lung cancer applications: for example these comprise one or more of the sequences identified as tryptic sequences in the 3^{rd} column of Table 91b;
for ovarian cancer applications: for example these comprise one or more of the sequences identified as tryptic sequences in the 3^{rd} column of Table 46;
for renal cell cancer applications: for example these comprise one or more of the sequences identified as tryptic sequences in the 3^{rd} column of Table 91a.

In relation to fragments, immunogenic fragments or antigenic fragments of OGTA257:
for colorectal cancer applications: for example these comprise one or more of the sequences identified as tryptic sequences in the 3^{rd} column of Table 92a;
for hepatocellular carcinoma applications: for example these comprise one or more of the sequences identified as tryptic sequences in the 3^{rd} column of Table 92b;
for lung cancer applications: for example these comprise one or more of the sequences identified as tryptic sequences in the 3^{rd} column of Table 92c;
for ovarian cancer applications: for example these comprise one or more of the sequences identified as tryptic sequences in the 3^{rd} column of Table 92d;
for pancreatic cancer applications: for example these comprise one or more of the sequences identified as tryptic sequences in the 3^{rd} column of Table 47.

In relation to fragments, immunogenic fragments or antigenic fragments of OGTA271:
for breast cancer applications: for example these comprise one or more of the sequences identified as tryptic sequences in the 3^{rd} column of Table 48a;
for cervical cancer applications: for example these comprise one or more of the sequences identified as tryptic sequences in the 3^{rd} column of Table 48b;
for colorectal cancer applications: for example these comprise one or more of the sequences identified as tryptic sequences in the 3^{rd} column of Table 48c and Table 93a;
for hepatocellular carcinoma applications: for example these comprise one or more of the sequences identified as tryptic sequences in the 3^{rd} column of Table 48d and table 93c;
for lung cancer applications: for example these comprise one or more of the sequences identified as tryptic sequences in the 3^{rd} column of Table 48e and Table 93d;
for osteosarcoma applications: for example these comprise one or more of the sequences identified as tryptic sequences in the 3^{rd} column of Table 48f;
for pancreatic cancer applications: for example these comprise one or more of the sequences identified as tryptic sequences in the 3^{rd} column of Table 48g;
for renal cell cancer applications: for example these comprise one or more of the sequences identified as tryptic sequences in the 3^{rd} column of Table 48f and table 93b.

The present invention additionally provides: (a) preparations comprising an isolated OGTA according to the invention; (b) preparations comprising one or more fragments of an OGTA according to the invention; and (c) antibodies or other affinity reagents that bind to an OGTA according to the invention, to fragments fragments thereof, or both. As used herein, OGTA(s) are “isolated” when they are present in preparations that are substantially free of contaminating proteins, i.e., preparations in which less than 10% by weight (for example less than 5%, such as less than 1%) of the total protein present is contaminating protein(s). A contaminating protein is a protein having a significantly different amino acid sequence from that of an isolated OGTA according to the invention, as determined by mass spectral analysis. As used herein, a “significantly different” sequence is one that permits the contaminating protein to be resolved from an OGTA of the invention by mass spectral analysis, performed according to the Reference Protocols.

An OGTA according to the invention can be assayed by any method known to those skilled in the art, including but not limited to, the Preferred Technologies described herein in Examples 1 and 2, kinase assays, enzyme assays, binding assays and other functional assays, immunoassays, and western blotting. In one embodiment, the OGTAs according to the invention are separated on 1-D gels by virtue of their MW and visualized by staining the gel. In one embodiment, OGTA(s) are stained with a fluorescent dye and imaged with a fluorescence scanner. Sypro Red (Molecular Probes, Inc., Eugene, Oreg.) is a suitable dye for this purpose. A preferred fluorescent dye is disclosed in U.S. application Ser. No. 09/412,168, filed on Oct. 5, 1999, which is incorporated herein by reference in its entirety. In another embodiment, OGTA(s) are analysed using isobaric tags for relative and absolute quantification (iTRAQ).

Alternatively, OGTAs according to the invention can be detected in an immunoassay. In one embodiment, an immunoassay is performed by contacting a sample from a subject to be tested with an anti-OGTA002, anti-OGTA009, anti-OGTA016, anti-OGTA028, anti-OGTA037, anti-OGTA041, anti-OGTA053, anti-OGTA054, anti-OGTA066, anti-OGTA072, anti-OGTA074, anti-OGTA076, anti-OGTA085, anti-OGTA087, anti-OGTA088, anti-OGTA089, anti-OGTA091, anti-OGTA098, anti-OGTA101, anti-OGTA104, anti-OGTA106, anti-OGTA112, anti-OGTA113, anti-OGTA119, anti-OGTA124, anti-OGTA126, anti-OGTA156, anti-OGTA159, anti-OGTA168, anti-OGTA169, anti-OGTA174, anti-OGTA176, anti-OGTA177, anti-OGTA197, anti-OGTA202, anti-OGTA203, anti-OGTA206, anti-OGTA213, anti-OGTA214, anti-OGTA216, anti-OGTA222, anti-OGTA236, anti-OGTA237, anti-OGTA247, anti-OGTA248, anti-OGTA249, anti-OGTA257 or anti-OGTA271 antibody (or other affinity reagent) under conditions such that immunospecific binding can occur if the relevant OGTA is present, and detecting or measuring the amount of any immunospecific binding by the affinity reagent. Anti-OGTA002, anti-OGTA009, anti-OGTA016, anti-OGTA028, anti-OGTA037, anti-OGTA041, anti-OGTA053, anti-OGTA054, anti-OGTA066, anti-OGTA072, anti-OGTA074, anti-OGTA076, anti-OGTA085, anti-OGTA087, anti-OGTA088, anti-OGTA089, anti-OGTA091, anti-OGTA098, anti-OGTA101, anti-OGTA104, anti-OGTA106, anti-OGTA112, anti-OGTA113, anti-OGTA119, anti-OGTA124, anti-OGTA126, anti-OGTA156, anti-OGTA159, anti-OGTA168, anti-OGTA169, anti-OGTA174, anti-OGTA176, anti-OGTA177, anti-OGTA197, anti-OGTA202, anti-OGTA203, anti-OGTA206, anti-OGTA213, anti-OGTA214, anti-OGTA216, anti-OGTA222, anti-OGTA236, anti-OGTA237, anti-OGTA247, anti-OGTA248, anti-OGTA249, anti-OGTA257 and anti-OGTA271 affinity reagents can be produced by the methods and techniques taught herein.

Proteins according to the invention may be detected by virtue of the detection of a fragment thereof e.g. an immunogenic or antigenic fragment thereof. Fragments may have a length of at least 10, more typically at least 20 amino acids e.g. at least 50 or 100 amino acids e.g. at least 200 or 500 amino acids e.g. at least 1000 amino acids e.g. at least 1500 amino acids e.g. at least 2000 amino acids.

In one embodiment one or more for example 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 proteins according to the invention are dectected using an appropriate array analysis.

In one embodiment, binding of antibody (or other affinity reagent) in tissue sections can be used to detect aberrant OGTA(s) localization or an aberrant level of OGTA(s). In a specific embodiment, an antibody (or other affinity reagent) to an OGTA according to the invention can be used to assay a patient tissue (e.g., a lymphoid, breast, cervical, colorectal, gastric, brain, liver, lung, skin, neuronal, osteoblast, ovarian, pancreatic, prostate, kidney or eye tissue) for the level of OGTA(s) where an aberrant level of OGTA(s) is indicative of a relevant cancer. As used herein, an “aberrant level” means a level that is increased compared with the level in a subject free from said cancer or a reference level. This embodiment may be suitable for detecting elevated levels of OGTA066 in cancer tissue, such as colorectal cancer, kidney cancer, lung cancer and/or pancreatic cancer tissue.

Any suitable immunoassay can be used, including, without limitation, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, a protein according to the invention can be detected in a fluid sample (e.g., blood, urine, or saliva) by means of a two-step sandwich assay. In the first step, a capture reagent (e.g., an anti-OGTA002, anti-OGTA009, anti-OGTA016, anti-OGTA028, anti-OGTA037, anti-OGTA041, anti-OGTA053, anti-OGTA054, anti-OGTA066, anti-OGTA072, anti-OGTA074, anti-OGTA076, anti-OGTA085, anti-OGTA087, anti-OGTA088, anti-OGTA089, anti-OGTA091, anti-OGTA098, anti-OGTA101, anti-OGTA104, anti-OGTA106, anti-OGTA112, anti-OGTA113, anti-OGTA119, anti-OGTA124, anti-OGTA126, anti-OGTA156, anti-OGTA159, anti-OGTA168, anti-OGTA169, anti-OGTA174, anti-OGTA176, anti-OGTA177, anti-OGTA197, anti-OGTA202, anti-OGTA203, anti-OGTA206, anti-OGTA213, anti-OGTA214, anti-OGTA216, anti-OGTA222, anti-OGTA236, anti-OGTA237, anti-OGTA247, anti-OGTA248, anti-OGTA249, anti-OGTA257 or anti-OGTA271 antibody or other affinity reagent) is used to capture the relevant OGTA(s). The capture reagent can optionally be immobilized on a solid phase. In the second step, a directly or indirectly labeled detection reagent is used to detect the captured OGTA(s). In one embodiment, the detection reagent is a lectin. Any lectin can be used for this purpose that preferentially binds to an OGTA according to the invention rather than to other isoforms that have the same core protein as an OGTA according to the invention or to other proteins that share the antigenic determinant recognized by the antibody. In one embodiment, the chosen lectin binds an OGTA according to the invention with at least 2-fold greater affinity, more for example at least 5-fold greater affinity, still more preferably at least 10-fold greater affinity, than to said other isoforms that have the same core protein as an OGTA according to the invention or to said other proteins that share the antigenic determinant recognized by the affinity reagent.

Whilst not wishing to be bound by theory it is thought that in patients with a relevant cancer such as colorectal cancer, kidney cancer, lung cancer and/or pancreatic cancer that the levels of OGTA066 are reduced in fluid samples such as serum samples in comparison to a subject free of said cancer. This is thought to be because the protein seems to be recruited to the cancerous tissue and thus the levels found in the cancerous tissue are higher in patients with the relevant cancer than subjects free from same.

Based on the present description, a lectin that is suitable for detecting an OGTA according to the invention can readily be identified by methods well known in the art, for instance upon testing one or more lectins enumerated in Table I on pages 158-159 of Sumar et al., Lectins as Indicators of Disease-Associated Glycoforms, In: Gabius H-J & Gabius S (eds.), 1993, Lectins and Glycobiology, at pp. 158-174 (which is incorporated herein by reference in its entirety). In an alternative embodiment, the detection reagent is an antibody (or other affinity reagent), e.g. an antibody that immunospecifically detects other post-translational modifications, such as an antibody that immunospecifically binds to phosphorylated amino acids. Examples of such antibodies include those that bind to phosphotyrosine (BD Transduction Laboratories, catalog nos.: P11230-050/P11230-150; P11120; P38820; P39020), those that bind to phosphoserine (Zymed Laboratories Inc., South San Francisco, Calif., catalog no. 61-8100) and those that bind to phosphothreonine (Zymed Laboratories Inc., South San Francisco, Calif., catalogue nos. 71-8200, 13-9200).

If desired, a gene encoding a protein according to the invention a related gene, or related nucleic acid sequences or subsequences, including complementary sequences, can also be used in hybridization assays. A nucleotide encoding a protein according to the invention, or subsequences thereof comprising at least 8 nucleotides, for example at least 12 nucleotides, and most preferably at least 15 nucleotides can be used as a hybridization probe. Hybridization assays can be used for detection, prognosis, diagnosis, or monitoring of conditions, disorders, or disease states, associated with aberrant expression of the gene encoding a protein according to the invention, or for differential diagnosis of subjects with signs or symptoms suggestive of a relevant cancer. In particular, such a hybridization assay can be carried out by a method comprising contacting a subject's sample containing nucleic acid with a nucleic acid probe capable of hybridizing to a DNA or RNA that encodes a protein according to the invention under conditions such that hybridization can occur, and detecting or measuring any resulting hybridization.

Hence nucleic acid encoding OGTA(s) (e.g. DNA or more suitably RNA) may be detected, for example, using a hybridizing agent capable of hybridizing to nucleic acid encoding OGTA.

One such exemplary method comprises:
(a) contacting one or more oligonucleotide probes comprising 10 or more consecutive nucleotides complementary to a nucleotide sequence encoding OGTA, with an RNA obtained from a biological sample from the subject or with cDNA copied from the RNA, wherein said contacting occurs under conditions that permit hybridization of the probe to the nucleotide sequence if present;
(b) detecting hybridization, if any, between the probe and the nucleotide sequence; and
(c) comparing the hybridization, if any, detected in step (b) with the hybridization detected in a control sample, or with a previously determined reference range.

Thus in one aspect the analysis for differential expression of the gene or proteins in performed using microarrays comprising probes sets, allowing simulateous analysis in relation to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 proteins according to the invention. This technology is well known in the relevant field.

The invention also provides diagnostic kits, comprising an anti-OGTA002, anti-OGTA009, anti-OGTA016, anti-OGTA028, anti-OGTA037, anti-OGTA041, anti-OGTA053, anti-OGTA054, anti-OGTA066, anti-OGTA072, anti-OGTA074, anti-OGTA076, anti-OGTA085, anti-OGTA087, anti-OGTA088, anti-OGTA089, anti-OGTA091, anti-OGTA098, anti-OGTA101, anti-OGTA104, anti-OGTA106, anti-OGTA112, anti-OGTA113, anti-OGTA119, anti-OGTA124, anti-OGTA126, anti-OGTA156, anti-OGTA159, anti-OGTA168, anti-OGTA169, anti-OGTA174, anti-OGTA176, anti-OGTA177, anti-OGTA197, anti-OGTA202, anti-OGTA203, anti-OGTA206, anti-OGTA213, anti-OGTA214, anti-OGTA216, anti-OGTA222, anti-OGTA236, anti-OGTA237, anti-OGTA247, anti-OGTA248, anti-OGTA249, anti-OGTA257 or anti-OGTA271 antibody (or other affinity reagent).

In addition, such a kit may optionally comprise one or more of the following: (1) instructions for using the anti-OGTA002, anti-OGTA009, anti-OGTA016, anti-OGTA028, anti-OGTA037, anti-OGTA041, anti-OGTA053, anti-OGTA054, anti-OGTA066, anti-OGTA072, anti-OGTA074, anti-OGTA076, anti-OGTA085, anti-OGTA087, anti-OGTA088, anti-OGTA089, anti-OGTA091, anti-OGTA098, anti-OGTA101, anti-OGTA104, anti-OGTA106, anti-OGTA112, anti-OGTA113, anti-OGTA119, anti-OGTA124, anti-OGTA126, anti-OGTA156, anti-OGTA159, anti-OGTA168, anti-OGTA169, anti-OGTA174, anti-OGTA176, anti-OGTA177, anti-OGTA197, anti-OGTA202, anti-OGTA203, anti-OGTA206, anti-OGTA213, anti-OGTA214, anti-OGTA216, anti-OGTA222, anti-OGTA236, anti-OGTA237, anti-OGTA247, anti-OGTA248, anti-OGTA249, anti-OGTA257 or anti-OGTA271 affinity reagent for diagnosis, prognosis, therapeutic monitoring or any combination of these applications; (2) a labeled binding partner to the affinity reagent; (3) a solid phase (such as a reagent strip) upon which the anti-OGTA002, anti-OGTA009, anti-OGTA016, anti-OGTA028, anti-OGTA037, anti-OGTA041, anti-OGTA053, anti-OGTA054, anti-OGTA066, anti-OGTA072, anti-OGTA074, anti-OGTA076, anti-OGTA085, anti-OGTA087, anti-OGTA088, anti-OGTA089, anti-OGTA091, anti-OGTA098, anti-OGTA101, anti-OGTA104, anti-OGTA106, anti-OGTA112, anti-OGTA113, anti-OGTA119, anti-OGTA124, anti-OGTA126, anti-OGTA156, anti-OGTA159, anti-OGTA168, anti-OGTA169, anti-OGTA174, anti-OGTA176, anti-OGTA177, anti-OGTA197, anti-OGTA202, anti-OGTA203, anti-OGTA206, anti-OGTA213, anti-OGTA214, anti-OGTA216, anti-OGTA222, anti-OGTA236, anti-OGTA237, anti-OGTA247, anti-OGTA248, anti-OGTA249, anti-OGTA257 or anti-OGTA271 affinity reagent is immobilized; and (4) a label or insert indicating regulatory approval for diagnostic, prognostic or therapeutic use or any combination thereof.

If no labeled binding partner to the affinity reagent is provided, the anti-OGTA002, anti-OGTA009, anti-OGTA016, anti-OGTA028, anti-OGTA037, anti-OGTA041, anti-OGTA053, anti-OGTA054, anti-OGTA066, anti-OGTA072, anti-OGTA074, anti-OGTA076, anti-OGTA085, anti-OGTA087, anti-OGTA088, anti-OGTA089, anti-OGTA091, anti-OGTA098, anti-OGTA101, anti-OGTA104, anti-OGTA106, anti-OGTA112, anti-OGTA113, anti-OGTA119, anti-OGTA124, anti-OGTA126, anti-OGTA156, anti-OGTA159, anti-OGTA168, anti-OGTA169, anti-OGTA174, anti-OGTA176, anti-OGTA177, anti-OGTA197, anti-OGTA202, anti-OGTA203, anti-OGTA206, anti-OGTA213, anti-OGTA214, anti-OGTA216, anti-OGTA222, anti-OGTA236, anti-OGTA237, anti-OGTA247, anti-OGTA248, anti-OGTA249, anti-OGTA257 or anti-OGTA271 affinity reagent itself can be labeled with a detectable marker, e.g. a chemiluminescent, enzymatic, fluorescent, or radioactive moiety.

The invention also provides a kit comprising a nucleic acid probe capable of hybridizing to RNA encoding a protein according to the invention. In a specific embodiment, a kit comprises in one or more containers a pair of primers (e.g. each in the size range of 6-30 nucleotides, more preferably 10-30 nucleotides and still more preferably 10-20 nucleotides) that under appropriate reaction conditions can prime amplification of at least a portion of a nucleic acid encoding a protein according to the invention, such as by polymerase chain reaction (see, e.g., Innis et al., 1990, PCR Protocols, Academic Press, Inc., San Diego, Calif.), ligase chain reaction (see EP 320,308) use of Qβ replicase, cyclic probe reaction, or other methods known in the art.

A kit can optionally further comprise a predetermined amount of an OGTA according to the invention or a nucleic acid encoding same, e.g. for use as a standard or control.

### Use in Clinical Studies

The diagnostic methods and compositions of the present invention can assist in monitoring a clinical study, e.g. to evaluate drugs for therapy of a relevant cancer. In one embodiment, candidate molecules are tested for their ability to restore levels of a protein(s) according to the invention in a subject having a relevant cancer to levels found in subjects free from said cancer or, in a treated subject, to preserve said levels at or near non-B-cell non-Hodgkin's lymphoma, non-breast cancer, non-cervical cancer, non-colorectal cancer, non-gastric cancer, non-glioblastoma, non-hepatocellular carcinoma, non-lung cancer, non-lymphoid leukaemia (particularly acute T-cell leukaemia and chronic lymphocytic leukaemia), non-melanoma, non-neuroblastoma, non-osteosarcoma, non-ovarian cancer, non-pancreatic cancer, non-prostate cancer, non-renal cell cancer or non-retinoblastoma values.

In another embodiment, the methods and compositions of the present invention are used to screen candidates for a clinical study to identify individuals having a relevant cancer; such individuals can then be excluded from the study or can be placed in a separate cohort for treatment or analysis.

### Production of Proteins of the Invention and Corresponding Nucleic Acid

A DNA of the present invention can be obtained by isolation as a cDNA fragment from cDNA libraries using as starter materials commercial mRNAs and determining and identifying the nucleotide sequences thereof. That is, specifically, clones are randomly isolated from cDNA libraries, which are prepared according to Ohara et al's method (DNA Research Vol. 4, 53-59 (1997)). Next, through hybridization, duplicated clones (which appear repeatedly) are removed and then in vitro transcription and translation are carried out. Nucleotide sequences of both termini of clones, for which products of 50 kDa or more are confirmed, are determined. Furthermore, databases of known genes are searched for homology using the thus obtained terminal nucleotide sequences as queries. The entire nucleotide sequence of a clone revealed to be novel as a result is determined. In addition to the above screening method, the 5′ and 3′ terminal sequences of cDNA are related to a human genome sequence. Then an unknown long-chain gene is confirmed in a region between the sequences, and the full-length of the cDNA is analyzed. In this way, an unknown gene that is unable to be obtained by a conventional cloning method that depends on known genes can be systematically cloned.

Moreover, all of the regions of a human-derived gene containing a DNA of the present invention can also be prepared using a PCR method such as RACE while paying sufficient attention to prevent artificial errors from taking place in short fragments or obtained sequences. As described above, clones having DNA of the present invention can be obtained. In another means for cloning DNA of the present invention, a synthetic DNA primer having an appropriate nucleotide sequence of a portion of a polypeptide of the present invention is produced, followed by amplification by the PCR method using an appropriate library.

Alternatively, selection can be carried out by hybridization of the DNA of the present invention with a DNA that has been incorporated into an appropriate vector and labeled with a DNA fragment or a synthetic DNA encoding some or all of the regions of the polypeptide of the present invention. Hybridization can be carried out by, for example, the method described in Current Protocols in Molecular Biology (edited by Frederick M. Ausubel et al., 1987). DNA of the present invention may be any DNA, as long as they contain nucleotide sequences encoding the polypeptides of the present invention as described above. Such a DNA may be a cDNA identified and isolated from cDNA libraries or the like that are derived from lymphoid, breast, cervical, colorectal, gastric, brain, liver, lung, skin, neuronal, osteoblast, ovarian, pancreatic, prostate, kidney or eye tissue. Such a DNA may also be a synthetic DNA or the like. Vectors for use in library construction may be any of bacteriophages, plasmids, cosmids, phargemids, or the like. Furthermore, by the use of a total RNA fraction or a mRNA fraction prepared from the above cells and/or tissues, amplification can be carried out by a direct reverse transcription coupled polymerase chain reaction (hereinafter abbreviated as “RT-PCR method”).

DNA encoding the above polypeptides consisting of amino acid sequences that are substantially identical to the amino acid sequences of OGTA(s) according to the invention or DNA encoding the above polypeptides consisting of amino acid sequences derived from the amino acid sequences of OGTA(s) by deletion, substitution, or addition of one or more amino acids composing a portion of the amino acid sequence can be easily produced by an appropriate combination of, for example, a site-directed mutagenesis method, a gene homologous recombination method, a primer elongation method, and the PCR method known by persons skilled in the art. In addition, at this time, a possible method for causing a polypeptide to have substantially equivalent biological activity is substitution of homologous amino acids (e.g. polar and nonpolar amino acids, hydrophobic and hydrophilic amino acids, positively-charged and negatively charged amino acids, and aromatic amino acids) among amino acids composing the polypeptide. Furthermore, to maintain substantially equivalent biological activity, amino acids within functional domains contained in each polypeptide of the present invention are for example conserved.

Furthermore, examples of DNA of the present invention include DNA comprising nucleotide sequences that encode the amino acid sequences of an OGTA according to the invention and DNA hybridizing under stringent conditions to the DNA and encoding polypeptides (proteins) having biological activity (functions) equivalent to the functions of the polypeptides consisting of the amino acid sequence of an OGTA according to the invention. Under such conditions, an example of such DNA capable of hybridizing to DNA comprising the nucleotide sequences that encode the amino acid sequence of a protein according to the invention is DNA comprising nucleotide sequences that have a degree of overall mean homology with the entire nucleotide sequence of the DNA, such as approximately 80% or more, for example approximately 90% or more, and more preferably approximately 95% or more. Hybridization can be carried out according to a method known in the art such as a method described in Current Protocols in Molecular Biology (edited by Frederick M. Ausubel et al., 1987) or a method according thereto. Here, “stringent conditions” are, for example, conditions of approximately “1*SSC, 0.1% SDS, and 37° C., more stringent conditions of approximately “0.5*SSC, 0.1% SDS, and 42° C., or even more stringent conditions of approximately “0.2*SSC, 0.1% SDS, and 65° C. With more stringent hybridization conditions, the isolation of a DNA having high homology with a probe sequence can be expected. The above combinations of SSC, SDS, and temperature conditions are given for illustrative purposes. Stringency similar to the above can be achieved by persons skilled in the art using an appropriate combination of the above factors or other factors (for example, probe concentration, probe length, and reaction time for hybridization) for determination of hybridization stringency.

A cloned DNA of the present invention can be directly used or used, if desired, after digestion with a restriction enzyme or addition of a linker, depending on purposes. The DNA may have ATG as a translation initiation codon at the 5′ terminal side and have TAA, TGA, or TAG as a translation termination codon at the 3′ terminal side. These translation initiation and translation termination codons can also be added using an appropriate synthetic DNA adapter.

In methods of the invention the OGTA employed is, for example provided in isolated form, such as a form where the polypeptide has been purified to at least to some extent. The polypeptide may be provided in substantially pure form, that is to say free, to a substantial extent, from other proteins. The polypeptide can also be produced using recombinant methods, synthetically produced or produced by a combination of these methods. OGTA(s) according to the invention can be easily prepared by any method known by persons skilled in the art, which involves producing an expression vector containing a DNA of the present invention or a gene containing a DNA of the present invention, culturing a transformant transformed using the expression vector, generating and accumulating a polypeptide of the present invention or a recombinant protein containing the polypeptide, and then collecting the resultant.

Recombinant OGTA polypeptide may be prepared by processes well known in the art from genetically engineered host cells comprising expression systems. Accordingly, the present invention also relates to expression systems which comprise an OGTA polypeptide or nucleic acid, to host cells which are genetically engineered with such expression systems and to the production of OGTA polypeptide by recombinant techniques. For recombinant polypeptide production, host cells can be genetically engineered to incorporate expression systems or portions thereof for nucleic acids. Such incorporation can be performed using methods well known in the art, such as, calcium phosphate transfection, DEAD-dextran mediated transfection, transvection, microinjection, cationic lipid-mediated transfection, electroporation, transduction, scrape loading, ballistic introduction or infection (see e.g. Davis et al., Basic Methods in Molecular Biology, 1986 and Sambrook et al., Molecular Cloning: A Laboratory Manual, 2nd Ed., Cold Spring Harbour laboratory Press, Cold Spring Harbour, N.Y., 1989).

As host cells, for example, bacteria of the genus Escherichia, Streptococci, Staphylococci, Streptomyces, bacteria of the genus Bacillus, yeast, Aspergillus cells, insect cells, insects, and animal cells are used. Specific examples of bacteria of the genus Escherichia, which are used herein, include Escherichia coli K12 and DH1 (Proc. Natl. Acad. Sci. U.S.A., Vol. 60, 160 (1968)), JM103 (Nucleic Acids Research, Vol. 9, 309 (1981)), JA221 (Journal of Molecular Biology, Vol. 120, 517 (1978)), and HB101 (Journal of Molecular Biology, Vol. 41, 459 (1969)). As bacteria of the genus Bacillus, for example, Bacillus subtilis MI114 (Gene, Vol. 24, 255 (1983)) and 207-21 (Journal of Biochemistry, Vol. 95, 87 (1984)) are used. As yeast, for example, Saccaromyces cerevisiae AH22, AH22R-, NA87-11A, DKD-5D, and 20B-12, Schizosaccaromyces pombe NCYC1913 and NCYC2036, and Pichia pastoris are used. As insect cells, for example, Drosophila S2 and Spodoptera Sf9 cells are used. As animal cells, for example, COS-7 and Vero monkey cells, CHO Chinese hamster cells (hereinafter abbreviated as CHO cells), dhfr-gene-deficient CHO cells, mouse L cells, mouse AtT-20 cells, mouse myeloma cells, rat GH3 cells, human FL cells, COS, HeLa, C127,3T3, HEK 293, BHK and Bowes melanoma cells are used.

Cell-free translation systems can also be employed to produce recombinant polypeptides (e.g. rabbit reticulocyte lysate, wheat germ lysate, SP6/T7 in vitro T&T and RTS100 E. Coli HY transcription and translation kits from Roche Diagnostics Ltd., Lewes, UK and the TNT Quick coupled Transcription/Translation System from Promega UK, Southampton, UK). The expression vector can be produced according to a method known in the art. For example, the vector can be produced by (1) excising a DNA fragment containing a DNA of the present invention or a gene containing a DNA of the present invention and (2) ligating the DNA fragment downstream of the promoter in an appropriate expression vector. A wide variety of expression systems can be used, such as and without limitation, chromosomal, episomal and virus-derived systems, e.g. plasmids derived from Escherichia coli (e.g. pBR322, pBR325, pUC18, and pUC118), plasmids derived from Bacillus subtilis (e.g. pUB110, pTPS, and pC194), from bacteriophage, from transposons, from yeast episomes (e.g. pSH19 and pSH15), from insertion elements, from yeast chromosomal elements, from viruses such as baculoviruses, papova viruses such as SV40, vaccinia viruses, adenoviruses, fowl pox viruses, pseudorabies viruses and retroviruses, and vectors derived from combinations thereof, such as those derived from plasmid and bacteriophage (such as [lambda] phage) genetic elements, such as cosmids and phagemids. The expression systems may contain control regions that regulate as well as engender expression. Promoters to be used in the present invention may be any promoters as long as they are appropriate for hosts to be used for gene expression. For example, when a host is Escherichia coli, a trp promoter, a lac promoter, a recA promoter, a pL promoter, an 1 pp promoter, and the like are preferred. When a host is Bacillus subtilis, an SPO1 promoter, an SPO2 promoter, a penP promoter, and the like are preferred. When a host is yeast, a PHOS promoter, a PGK promoter, a GAP promoter, an ADH promoter, and the like are preferred. When an animal cell is used as a host, examples of promoters for use in this case include an SRa promoter, an SV40 promoter, an LTR promoter, a CMV promoter, and an HSV-TK promoter. Generally, any system or vector that is able to maintain, propagate or express a nucleic acid to produce a polypeptide in a host may be used.

The appropriate nucleic acid sequence may be inserted into an expression system by any variety of well known and routine techniques, such as those set forth in Sambrook et al., supra. Appropriate secretion signals may be incorporated into the OGTA polypeptide to allow secretion of the translated protein into the lumen of the endoplasmic reticulum, the periplasmic space or the extracellular environment. These signals may be endogenous to the OGTA polypeptide or they may be heterologous signals. Transformation of the host cells can be carried out according to methods known in the art. For example, the following documents can be referred to: Proc. Natl. Acad. Sci. U.S.A., Vol. 69, 2110 (1972); Gene, Vol. 17, 107 (1982); Molecular & General Genetics, Vol. 168, 111 (1979); Methods in Enzymology, Vol. 194, 182-187 (1991); Proc. Natl. Acad. Sci. U.S.A.), Vol. 75, 1929 (1978); Cell Technology, separate volume 8, New Cell Technology, Experimental Protocol. 263-267 (1995) (issued by Shujunsha); and Virology, Vol. 52, 456 (1973). The thus obtained transformant transformed with an expression vector containing a DNA of the present invention or a gene containing a DNA of the present invention can be cultured according to a method known in the art. For example, when hosts are bacteria of the genus Escherichia, the bacteria are generally cultured at approximately 15° C. to 43° C. for approximately 3 to 24 hours. If necessary, aeration or agitation can also be added. When hosts are bacteria of the genus Bacillus, the bacteria are generally cultured at approximately 30° C. to 40° C. for approximately 6 to 24 hours. If necessary, aeration or agitation can also be added. When transformants whose hosts are yeast are cultured, culture is generally carried out at approximately 20° C. to 35° C. for approximately 24 to 72 hours using media with pH adjusted to be approximately 5 to 8. If necessary, aeration or agitation can also be added. When transformants whose hosts are animal cells are cultured, the cells are generally cultured at approximately 30° C. to 40° C. for approximately 15 to 60 hours using media with the pH adjusted to be approximately 6 to 8. If necessary, aeration or agitation can also be added.

If an OGTA(s) polypeptide is to be expressed for use in cell-based screening assays, it is preferred that the polypeptide be produced at the cell surface. In this event, the cells may be harvested prior to use in the screening assay. If the OGTA polypeptide is secreted into the medium, the medium can be recovered in order to isolate said polypeptide. If produced intracellularly, the cells must first be lysed before the OGTA polypeptide is recovered. OGTA polypeptide can be recovered and purified from recombinant cell cultures or from other biological sources by well known methods including, ammonium sulphate or ethanol precipitation, acid extraction, anion or cation exchange chromatography, phosphocellulose chromatography, affinity chromatography, hydrophobic interaction chromatography, hydroxylapatite chromatography, molecular sieving chromatography, centrifugation methods, electrophoresis methods and lectin chromatography. In one embodiment, a combination of these methods is used. In another embodiment, high performance liquid chromatography is used. In a further embodiment, an antibody which specifically binds to an OGTA polypeptide according to the invention can be used to deplete a sample comprising an a relevant OGTA polypeptide of said polypeptide or to purify said polypeptide.

To separate and purify a polypeptide or a protein of the present invention from the culture products, for example, after culture, microbial bodies or cells are collected by a known method, they are suspended in an appropriate buffer, the microbial bodies or the cells are disrupted by, for example, ultrasonic waves, lysozymes, and/or freeze-thawing, the resultant is then subjected to centrifugation or filtration, and then a crude extract of the protein can be obtained. The buffer may also contain a protein denaturation agent such as urea or guanidine hydrochloride or a surfactant such as Triton X-100™. When the protein is secreted in a culture solution, microbial bodies or cells and a supernatant are separated by a known method after the completion of culture and then the supernatant is collected. The protein contained in the thus obtained culture supernatant or the extract can be purified by an appropriate combination of known separation and purification methods. The thus obtained polypeptide (protein) of the present invention can be converted into a salt by a known method or a method according thereto. Conversely, when the polypeptide (protein) of the present invention is obtained in the form of a salt, it can be converted into a free protein or peptide or another salt by a known method or a method according thereto. Moreover, an appropriate protein modification enzyme such as trypsin or chymotrypsin is caused to act on a protein produced by a recombinant before or after purification, so that modification can be arbitrarily added or a polypeptide can be partially removed. The presence of a polypeptide (protein) of the present invention or a salt thereof can be measured by various binding assays, enzyme immunoassays using specific antibodies, and the like.

Techniques well known in the art, may be used for refolding to regenerate native or active conformations of the OGTA polypeptide when the polypeptide has been denatured during isolation and or purification. In the context of the present invention, OGTA polypeptide can be obtained from a biological sample from any source, such as and without limitation, a blood sample or tissue sample, e.g. lymphoid, breast, cervical, colorectal, gastric, brain, liver, lung, skin, neuronal, osteoblast, ovarian, pancreatic, prostate, kidney or eye tissue sample.

OGTA polypeptide may be in the form of a “mature protein” or may be part of a larger protein such as a fusion protein. It is often advantageous to include an additional amino acid sequence which contains secretory or leader sequences, a pre-, pro- or prepro-protein sequence, or a sequence which aids in purification such as an affinity tag, for example, but without limitation, multiple histidine residues, a FLAG tag, HA tag or myc tag.

An additional sequence that may provide stability during recombinant production may also be used. Such sequences may be optionally removed as required by incorporating a cleavable sequence as an additional sequence or part thereof. Thus, an OGTA polypeptide may be fused to other moieties including other polypeptides or proteins (for example, glutathione S-transferase and protein A). Such a fusion protein can be cleaved using an appropriate protease, and then separated into each protein. Such additional sequences and affinity tags are well known in the art. In addition to the above, features known in the art, such as an enhancer, a splicing signal, a polyA addition signal, a selection marker, and an SV40 replication origin can be added to an expression vector, if desired.

In one embodiment one or more proteins according to the invention or fragments thereof, for example one or more sequences described herein may be fused with a heterologous fusion partner such as the surface protein, known as protein D from Haemophilus Influenza B, a non-structural protein from influenzae virus such as NS1, the S antigen from Hepatitis B or a protein known as LYTA such as the C terminal thereof.

### Production of Affinity Reagents to the Proteins of the Invention

According to those in the art, there are three main types of immunoaffinity reagentmonoclonal antibodies, phage display antibodies and smaller antibody-derived molecules such as Affibodies, Domain Antibodies (dAbs), Nanobodies or UniBodies. In general in applications according to the present invention where the use of antibodies is stated, other affinity reagents (e.g. Affibodies, domain antibodies, Nanobodies or UniBodies) may be employed. Such substances may be said to be capable of immunospecific binding to the proteins of the invention. Where appropriate the term “affinity agent” shall be construed to embrace immunoaffinity reagents and other substances capable of specific binding to the proteins of the invention including but not limited to ligands, lectins, streptavidins, antibody mimetics and synthetic binding agents.

### Production of Antibodies to the Proteins of the Invention

According to the invention OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, an analog of any of same, a related protein or a fragment or derivative of any of the foregoing may be used as an immunogen to generate antibodies which immunospecifically bind such an immunogen. Such immunogens can be isolated by any convenient means, including the methods described above. The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.” Antibodies of the invention include, but are not limited to polyclonal, monoclonal, bispecific, humanized or chimeric antibodies, single chain antibodies, Fab fragments and F(ab′)₂ fragments, fragments produced by a Fab expression library, anti-idiotypic (anti-Id) antibodies, and epitope-binding fragments of any of the above. The immunoglobulin molecules of the invention can be of any class (e.g., IgG, IgE, IgM, IgD and IgA) or subclass of immunoglobulin molecule.

The term “specifically binds” (or “immunospecifically binds”) is not intended to indicate that an antibody binds exclusively to its intended target. Rather, an antibody “specifically binds” if its affinity for its intended target is about 5-fold greater when compared to its affinity for a non-target molecule. For example the affinity of the antibody will be at least about 5 fold, for example 10 fold, such as 25-fold, particularly 50-fold, and especially 100-fold or more, greater for a target molecule than its affinity for a non-target molecule. In some embodiments, specific binding between an antibody or other binding agent and an antigen means a binding affinity of at least 10⁶ M⁻¹. Antibodies may bind with affinities of at least about 10⁷ M⁻¹, such as between about 10⁸ M⁻¹ to about 10⁹ M⁻¹, about 10⁹ M⁻¹ to about 10¹⁰ M⁻¹, or about 10¹⁰ M⁻¹ to about 10¹¹ M⁻¹.

Affinity is calculated as K_{d}=k_{off}/kₒₙ (k_{off} is the dissociation rate constant, kₒₙ is the association rate constant and K_{d} is the equilibrium constant. Affinity can be determined at equilibrium by measuring the fraction bound (r) of labeled ligand at various concentrations (c). The data are graphed using the Scatchard equation: r/c=K(n−r):

where

r=moles of bound ligand/mole of receptor at equilibrium;

c=free ligand concentration at equilibrium;

K=equilibrium association constant; and

n=number of ligand binding sites per receptor molecule

By graphical analysis, r/c is plotted on the Y-axis versus r on the X-axis thus producing a Scatchard plot. The affinity is the negative slope of the line. k_{off} can be determined by competing bound labeled ligand with unlabeled excess ligand (see, e.g., U.S. Pat. No. 6,316,409). The affinity of a targeting agent for its target molecule is for example at least about 1×10⁻⁶ moles/liter, is more preferably at least about 1×10⁻⁷ moles/liter, is even more preferably at least about 1×10⁻⁸ moles/liter, is yet even more preferably at least about 1×10⁻⁹ moles/liter, and is most preferably at least about 1×10⁻¹⁰ moles/liter. Antibody affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988.

In one embodiment, antibodies that recognize gene products of genes encoding a protein of the invention are publicly available. In another embodiment, methods known to those skilled in the art are used to produce antibodies that recognize OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, an analog thereof, a related polypeptide, or a fragment or derivative of any of the foregoing. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In one embodiment of the invention, antibodies to a specific domain of an OGTA according to the invention are produced. In a specific embodiment, hydrophilic fragments of protein according to the invention are used as immunogens for antibody production.

In the production of antibodies, screening for the desired antibody can be accomplished by techniques known in the art, e.g. ELISA (enzyme-linked immunosorbent assay). For example, to select antibodies which recognize a specific domain of a protein according to the invention, one may assay generated hybridomas for a product which binds to an OGTA fragment containing such domain. For selection of an antibody that specifically binds a first OGTA homolog but which does not specifically bind to (or binds less avidly to) a second OGTA homolog, one can select on the basis of positive binding to the first OGTA homolog and a lack of binding to (or reduced binding to) the second OGTA homolog. Similarly, for selection of an antibody that specifically binds an OGTA according to the invention but which does not specifically bind to (or binds less avidly to) a different isoform of the same protein (such as a different glycoform having the same core peptide as an OGTA according to the invention, one can select on the basis of positive binding to an OGTA according to the invention and a lack of binding to (or reduced binding to) the different isoform (e.g., a different glycoform). Thus, the present invention provides an antibody (for example a monoclonal antibody) that binds with greater affinity (for example at least 2-fold, such as at least 5-fold, particularly at least 10-fold greater affinity) to OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 than to a different isoform or isoforms (e.g., glycoforms) of same.

Polyclonal antibodies which may be used in the methods of the invention are heterogeneous populations of antibody molecules derived from the sera of immunized animals. Unfractionated immune serum can also be used. Various procedures known in the art may be used for the production of polyclonal antibodies to OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, a fragment thereof, a related polypeptide, or a fragment of an OGTA related polypeptide. For example, one way is to purify polypeptides of interest or to synthesize the polypeptides of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm. Bull. (Tokyo) 38: 1192-99, 1990; Mostafavi et al., Biomed. Pept. Proteins Nucleic Acids 1: 255-60, 1995; Fujiwara et al., Chem. Pharm. Bull. (Tokyo) 44: 1326-31, 1996. The selected polypeptides may then be used to immunize by injection various host animals, including but not limited to rabbits, mice, rats, etc., to generate polyclonal or monoclonal antibodies. The Preferred Technology described herein in Example 1 provides isolated OGTA suitable for such immunization. If an OGTA is purified by gel electrophoresis, it can be used for immunization with or without prior extraction from the polyacrylamide gel. Various adjuvants (i.e. immunostimulants) may be used to enhance the immunological response, depending on the host species, including, but not limited to, complete or incomplete Freund's adjuvant, a mineral gel such as aluminum hydroxide, surface active substance such as lysolecithin, pluronic polyol, a polyanion, a peptide, an oil emulsion, keyhole limpet hemocyanin, dinitrophenol, and an adjuvant such as BCG (bacille Calmette-Guerin) or corynebacterium parvum. Additional adjuvants are also well known in the art.

For preparation of monoclonal antibodies (mAbs) directed toward proteins of the invention, a fragment thereof, a related polypeptide, or a fragment of a related polypeptide, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs of the invention may be cultivated in vitro or in vivo. In an additional embodiment of the invention, monoclonal antibodies can be produced in germ-free animals utilizing known technology (PCT/US90/02545, incorporated herein by reference).

The monoclonal antibodies include but are not limited to human monoclonal antibodies and chimeric monoclonal antibodies (e.g., human-mouse chimeras). A chimeric antibody is a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarity determining regions (CDRs) from the non-human species and a framework region from a human immunoglobulin molecule. (See, e.g., Queen, U.S. Pat. No. 5,585,089, which is incorporated herein by reference in its entirety.)

Chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, Bio/Techniques 4:214; U.S. Pat. No. 5,225,539; Jones et al., 1986, Nature 321:552-525; Verhoeyan et al. (1988) Science 239:1534; and Beidler et al., 1988, J. Immunol. 141:4053-4060.

Completely human antibodies are for example desirable for therapeutic treatment of human subjects.

Antibodies can be produced using transgenic mice which are incapable of expressing endogenous immunoglobulin heavy and light chain genes, but which can express human heavy and light chain genes. The transgenic mice are immunized in the normal fashion with a selected antigen, e.g., all or a portion of a protein according to the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce useful (including therapeutically useful) IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g.,U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903). The antibodies of the present invention can also be generated by the use of phage display technology to produce and screen libraries of polypeptides for binding to a selected target. See, e.g., Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims. In particular, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT Application No. PCT/GB91/01134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

The invention further provides for the use of bispecific antibodies, which can be made by methods known in the art. Traditional production of full length bispecific antibodies is based on the coexpression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published 13 May 1993, and in Traunecker et al., 1991, EMBO J. 10:3655-3659.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details for generating bispecific antibodies see, for example, Suresh et al., Methods in Enzymology, 1986, 121:210.

The invention provides functionally active fragments, derivatives or analogs of anti-OGTA002, anti-OGTA009, anti-OGTA016, anti-OGTA028, anti-OGTA037, anti-OGTA041, anti-OGTA053, anti-OGTA054, anti-OGTA066, anti-OGTA072, anti-OGTA074, anti-OGTA076, anti-OGTA085, anti-OGTA087, anti-OGTA088, anti-OGTA089, anti-OGTA091, anti-OGTA098, anti-OGTA101, anti-OGTA104, anti-OGTA106, anti-OGTA112, anti-OGTA113, anti-OGTA119, anti-OGTA124, anti-OGTA126, anti-OGTA156, anti-OGTA159, anti-OGTA168, anti-OGTA169, anti-OGTA174, anti-OGTA176, anti-OGTA177, anti-OGTA197, anti-OGTA202, anti-OGTA203, anti-OGTA206, anti-OGTA213, anti-OGTA214, anti-OGTA216, anti-OGTA222, anti-OGTA236, anti-OGTA237, anti-OGTA247, anti-OGTA248, anti-OGTA249, anti-OGTA257 or anti-OGTA271 immunoglobulin molecules.

Functionally active means that the fragment, derivative or analog is able to elicit anti-anti-idiotype antibodies (i.e., tertiary antibodies) that recognize the same antigen that is recognized by the antibody from which the fragment, derivative or analog is derived. Specifically, in a preferred embodiment the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art.

The present invention provides antibody fragments such as, but not limited to, F(ab′)₂ fragments and Fab fragments. Antibody fragments which recognize specific epitopes may be generated by known techniques. F(ab′)₂ fragments consist of the variable region, the light chain constant region and the CH1 domain of the heavy chain and are generated by pepsin digestion of the antibody molecule. Fab fragments are generated by reducing the disulfide bridges of the F(ab′)₂ fragments. The invention also provides heavy chain and light chain dimers of the antibodies of the invention, or any minimal fragment thereof such as Fvs or single chain antibodies (SCAs) (e.g., as described in U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54), or any other molecule with the same specificity as the antibody of the invention. Single chain antibodies are formed by linking the heavy and light chain fragments of the Fv region via an amino acid bridge, resulting in a single chain polypeptide. Techniques for the assembly of functional Fv fragments in E. coli may be used (Skerra et al., 1988, Science 242:1038-1041).

In other embodiments, the invention provides fusion proteins of the immunoglobulins of the invention (or functionally active fragments thereof), for example in which the immunoglobulin is fused via a covalent bond (e.g., a peptide bond), at either the N-terminus or the C-terminus to an amino acid sequence of another protein (or portion thereof, for example at least 10, 20 or 50 amino acid portion of the protein) that is not the immunoglobulin. For example the immunoglobulin, or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

The immunoglobulins of the invention include analogs and derivatives that are modified, i.e., by the covalent attachment of any type of molecule as long as such covalent attachment does not impair immunospecific binding. For example, but not by way of limitation, the derivatives and analogs of the immunoglobulins include those that have been further modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the analog or derivative may contain one or more non-classical amino acids.

The foregoing antibodies can be used in methods known in the art relating to the localization and activity of an OGTA, e.g., for imaging this protein, measuring levels thereof in appropriate physiological samples, in diagnostic methods, etc.

### Production of Affibodies to the Proteins of the Invention

Affibody molecules represent a new class of affinity proteins based on a 58-amino acid residue protein domain, derived from one of the IgG-binding domains of staphylococcal protein A. This three helix bundle domain has been used as a scaffold for the construction of combinatorial phagemid libraries, from which Affibody variants that target the desired molecules can be selected using phage display technology (Nord K, Gunneriusson E, Ringdahl J, Stahl S, Uhlen M, Nygren P A, Binding proteins selected from combinatorial libraries of an α-helical bacterial receptor domain, Nat Biotechnol 1997; 15:772-7. Ronmark J, Gronlund H, Uhlen M, Nygren P A, Human immunoglobulin A (IgA)-specific ligands from combinatorial engineering of protein A, Eur J Biochem 2002; 269:2647-55.). The simple, robust structure of Affibody molecules in combination with their low molecular weight (6 kDa), make them suitable for a wide variety of applications, for instance, as detection reagents (Ronmark J, Hansson M, Nguyen T, et al, Construction and characterization of affibody-Fc chimeras produced in Escherichia coli, J Immunol Methods 2002; 261:199-211) and to inhibit receptor interactions (Sandstorm K, Xu Z, Forsberg G, Nygren P A, Inhibition of the CD28-CD80 co-stimulation signal by a CD28-binding Affibody ligand developed by combinatorial protein engineering, Protein Eng 2003; 16:691-7). Further details of Affibodies and methods of production thereof may be obtained by reference to U.S. Pat. No. 5,831,012 which is herein incorporated by reference in its entirety.

Labelled Affibodies or the like may also be useful in imaging applications for determining abundance of Isoforms.

### Production of Domain Antibodies to the Proteins of the Invention

References to antibodies herein embrace references to Domain Antibodies. Domain Antibodies (dAbs) are the smallest functional binding units of antibodies, corresponding to the variable regions of either the heavy (VH) or light (VL) chains of human antibodies. Domain Antibodies have a molecular weight of approximately 13 kDa. Domantis has developed a series of large and highly functional libraries of fully human VH and VL dAbs (more than ten billion different sequences in each library), and uses these libraries to select dAbs that are specific to therapeutic targets. In contrast to many conventional antibodies, Domain Antibodies are well expressed in bacterial, yeast, and mammalian cell systems. Further details of domain antibodies and methods of production thereof may be obtained by reference to U.S. Pat. Nos. 6,291,158; 6,582,915; 6,593,081; 6,172,197; 6,696,245; US Serial No. 2004/0110941; European patent application No. 1433846 and European Patents 0368684 & 0616640; WO05/035572, WO04/101790, WO04/081026, WO04/058821, WO04/003019 and WO03/002609, each of which is herein incorporated by reference in its entirety.

### Production of Nanobodies to the Proteins of the Invention

Nanobodies are antibody-derived therapeutic proteins that contain the unique structural and functional properties of naturally-occurring heavy-chain antibodies. These heavy-chain antibodies contain a single variable domain (VHH) and two constant domains (C_{H}2 and C_{H}3). Importantly, the cloned and isolated VHH domain is a perfectly stable polypeptide harbouring the full antigen-binding capacity of the original heavy-chain antibody. Nanobodies have a high homology with the VH domains of human antibodies and can be further humanised without any loss of activity. Importantly, Nanobodies have a low immunogenic potential, which has been confirmed in primate studies with Nanobody lead compounds.

Nanobodies combine the advantages of conventional antibodies with important features of small molecule drugs. Like conventional antibodies, Nanobodies show high target specificity, high affinity for their target and low inherent toxicity. However, like small molecule drugs they can inhibit enzymes and readily access receptor clefts. Furthermore, Nanobodies are extremely stable, can be administered by means other than injection (see e.g. WO 04/041867, which is herein incorporated by reference in its entirety) and are easy to manufacture. Other advantages of Nanobodies include recognising uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and are efficiently produced in almost all prokaryotic and eukaryotic hosts e.g. E. coli (see e.g. U.S. Pat. No. 6,765,087, which is herein incorporated by reference in its entirety), moulds (for example Aspergillus or Trichoderma) and yeast (for example Saccharomyces, Kluyveromyces, Hansenula or Pichia) (see e.g. U.S. Pat. No. 6,838,254, which is herein incorporated by reference in its entirety). The production process is scalable and multi-kilogram quantities of Nanobodies have been produced. Because Nanobodies exhibit a superior stability compared with conventional antibodies, they can be formulated as a long shelf-life, ready-to-use solution.

The Nanoclone method (see e.g. WO 06/079372, which is herein incorporated by reference in its entirety) is a proprietary method for generating Nanobodies against a desired target, based on automated high-throughout selection of B-cells.

### Production of UniBodies to the Proteins of the Invention

UniBody is a new proprietary antibody technology that creates a stable, smaller antibody format with an anticipated longer therapeutic window than current small antibody formats. IgG4 antibodies are considered inert and thus do not interact with the immune system. Genmab modified fully human IgG4 antibodies by eliminating the hinge region of the antibody. Unlike the full size IgG4 antibody, the half molecule fragment is very stable and is termed a UniBody. Halving the IgG4 molecule left only one area on the UniBody that can bind to disease targets and the UniBody therefore binds univalently to only one site on target cells. This univalent binding does not stimulate cancer cells to grow like bivalent antibodies might and opens the door for treatment of some types of cancer which ordinary antibodies cannot treat.

The UniBody is about half the size of a regular IgG4 antibody. This small size can be a great benefit when treating some forms of cancer, allowing for better distribution of the molecule over larger solid tumors and potentially increasing efficacy. Fabs typically do not have a very long half-life. UniBodies, however, were cleared at a similar rate to whole IgG4 antibodies and were able to bind as well as whole antibodies and antibody fragments in pre-clinical studies. Other antibodies primarily work by killing the targeted cells whereas UniBodies only inhibit or silence the cells.

Further details of UniBodies may be obtained by reference to patent WO2007/059782, which is herein incorporated by reference in its entirety.

### Expression of Affinity Reagents

### Expression of Antibodies

The antibodies of the invention can be produced by any method known in the art for the synthesis of antibodies, in particular, by chemical synthesis or by recombinant expression, and are for example produced by recombinant expression techniques.

Recombinant expression of antibodies, or fragments, derivatives or analogs thereof, requires construction of a nucleic acid that encodes the antibody. If the nucleotide sequence of the antibody is known, a nucleic acid encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding antibody, annealing and ligation of those oligonucleotides, and then amplification of the ligated oligonucleotides by PCR.

Alternatively, the nucleic acid encoding the antibody may be obtained by cloning the antibody. If a clone containing the nucleic acid encoding the particular antibody is not available, but the sequence of the antibody molecule is known, a nucleic acid encoding the antibody may be obtained from a suitable source (e.g., an antibody cDNA library, or cDNA library generated from any tissue or cells expressing the antibody) by PCR amplification using synthetic primers hybridizable to the 3′ and 5′ ends of the sequence or by cloning using an oligonucleotide probe specific for the particular gene sequence.

If an antibody molecule that specifically recognizes a particular antigen is not available (or a source for a cDNA library for cloning a nucleic acid encoding such an antibody), antibodies specific for a particular antigen may be generated by any method known in the art, for example, by immunizing an animal, such as a rabbit, to generate polyclonal antibodies or, more preferably, by generating monoclonal antibodies. Alternatively, a clone encoding at least the Fab portion of the antibody may be obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

Once a nucleic acid encoding at least the variable domain of the antibody molecule is obtained, it may be introduced into a vector containing the nucleotide sequence encoding the constant region of the antibody molecule (see, e.g., PCT Publication WO 86/05807; PCT Publication WO 89/01036; and U.S. Pat. No. 5,122,464). Vectors containing the complete light or heavy chain for co-expression with the nucleic acid to allow the expression of a complete antibody molecule are also available. Then, the nucleic acid encoding the antibody can be used to introduce the nucleotide substitution(s) or deletion(s) necessary to substitute (or delete) the one or more variable region cysteine residues participating in an intrachain disulfide bond with an amino acid residue that does not contain a sulfhydyl group. Such modifications can be carried out by any method known in the art for the introduction of specific mutations or deletions in a nucleotide sequence, for example, but not limited to, chemical mutagenesis, in vitro site directed mutagenesis (Hutchinson et al., 1978, J. Biol. Chem. 253:6551), PCT based methods, etc.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human antibody constant region, e.g., humanized antibodies.

Once a nucleic acid encoding an antibody molecule of the invention has been obtained, the vector for the production of the antibody molecule may be produced by recombinant DNA technology using techniques well known in the art. Thus, methods for preparing the proteins of the invention by expressing nucleic acid containing the antibody molecule sequences are described herein. Methods which are well known to those skilled in the art can be used to construct expression vectors containing antibody molecule coding sequences and appropriate transcriptional and translational control signals. These methods include, for example, in vitro recombinant DNA techniques, synthetic techniques, and in vivo genetic recombination. See, for example, the techniques described in Sambrook et al. (1990, Molecular Cloning, A Laboratory Manual, 2^{nd} Ed., Cold Spring Harbor Laboratory, Cold Spring Harbor, N.Y.) and Ausubel et al. (eds., 1998, Current Protocols in Molecular Biology, John Wiley & Sons, NY).

The expression vector is transferred to a host cell by conventional techniques and the transfected cells are then cultured by conventional techniques to produce an antibody of the invention.

The host cells used to express a recombinant antibody of the invention may be either bacterial cells such as Escherichia coli, or, for example, eukaryotic cells, especially for the expression of whole recombinant antibody molecule. In particular, mammalian cells such as Chinese hamster ovary cells (CHO), in conjunction with a vector such as the major intermediate early gene promoter element from human cytomegalovirus are an effective expression system for antibodies (Foecking et al., 1986, Gene 45:101; Cockett et al., 1990, Bio/Technology 8:2).

A variety of host-expression vector systems may be utilized to express an antibody molecule of the invention. Such host-expression systems represent vehicles by which the coding sequences of interest may be produced and subsequently purified, but also represent cells which may, when transformed or transfected with the appropriate nucleotide coding sequences, express the antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing the antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the antibody molecule being expressed. For example, when a large quantity of such a protein is to be produced, for example for the generation of pharmaceutical compositions comprising an antibody molecule, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter). In mammalian host cells, a number of viral-based expression systems (e.g., an adenovirus expression system) may be utilized.

As discussed above, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein.

For long-term, high-yield production of recombinant antibodies, stable expression is preferred. For example, cell lines that stably express an antibody of interest can be produced by transfecting the cells with an expression vector comprising the nucleotide sequence of the antibody and the nucleotide sequence of a selectable (e.g., neomycin or hygromycin), and selecting for expression of the selectable marker. Such engineered cell lines may be particularly useful in screening and evaluation of compounds that interact directly or indirectly with the antibody molecule.

The expression levels of the antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once an antibody molecule of the invention has been recombinantly expressed, it may be purified by any method known in the art for purification of an antibody molecule, for example, by chromatography (e.g., ion exchange chromatography, affinity chromatography such as with protein A or specific antigen, and sizing column chromatography), centrifugation, differential solubility, or by any other standard technique for the purification of proteins.

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccinia virus are loaded onto Ni²⁺ nitriloacetic acid-agarose columns and histidine-tagged proteins are selectively eluted with imidazole-containing buffers. The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) is present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

Those skilled in the art will recognise that many approaches can be taken in producing antibodies or binding fragments and screening and selecting for affinity and specificity for the various polypeptides, but these approaches do not change the scope of the invention.

For some applications (including therpeutica applications), antibodies (particularly monoclonal antibodies) may suitably be human or humanized animal (e.g. mouse) antibodies. Animal antibodies may be raised in animals using the human protein (e.g. OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271) as immunogen. Humanisation typically involves grafting CDRs identified thereby into human framework regions. Normally some subsequent retromutation to optimize the conformation of chains is required. Such processes are known to persons skilled in the art.

### Expression of Affibodies

The construction of affibodies has been described elsewhere (Ronnmark J, Gronlund H, Uhle' n, M., Nygren P. A°, Human immunoglobulin A (IgA)-specific ligands from combinatorial engineering of protein A, 2002, Eur. J. Biochem. 269, 2647-2655.), including the construction of affibody phage display libraries (Nord, K., Nilsson, J., Nilsson, B., Uhle' n, M. & Nygren, P. A, A combinatorial library of an a-helical bacterial receptor domain, 1995, Protein Eng. 8, 601-608. Nord, K., Gunneriusson, E., Ringdahl, J., Sta° hl, S., Uhle' n, M. & Nygren, P. A°, Binding proteins selected from combinatorial libraries of an a-helical bacterial receptor domain, 1997, Nat. Biotechnol. 15, 772-777.)

The biosensor analyses to investigate the optimal affibody variants using biosensor binding studies has also been described elsewhere (Ronnmark J, Gronlund H, Uhle' n, M., Nygren P. A°, Human immunoglobulin A (IgA)-specific ligands from combinatorial engineering of protein A, 2002, Eur. J. Biochem. 269, 2647-2655.).

### Affinity Reagent Modifications

In a preferred embodiment, affinity reagents such as antibodies or fragments thereof are conjugated to a diagnostic moiety (such as a detectable label) or a therapeutic moiety. The antibodies can be used for diagnosis or to determine the efficacy of a given treatment regimen. Detection can be facilitated by coupling the antibody to a detectable substance (label). Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics according to the present invention. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc. ⁶⁸Ga may also be employed.

As indicated above affinity reagents, such as antibodies for use in the invention, may be conjugated to a therapeutic moiety, such as a cytotoxin, a drug (e.g. an immunosuppressant) or a radiotoxin. Such conjugates are referred to herein as “immunoconjugates”. Immunoconjugates that include one or more cytotoxins are referred to as “immunotoxins”. A cytotoxin or cytotoxic agent includes any agent that is detrimental to (e.g. kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, and anthramycin (AMC)), and anti-mitotic agents (e.g. vincristine and vinblastine).

Other preferred examples of therapeutic cytotoxins that can be conjugated to an antibody of the invention include duocarmycins, calicheamicins, maytansines and auristatins, and derivatives thereof. An example of a calicheamicin antibody conjugate is commercially available (Mylotarg®; American Home Products).

Cytotoxins can be conjugated to antibodies of the invention using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumour tissue such as cathepsins (e.g. cathepsins B, C, D).

Examples of cytotoxins are described, for example, in U.S. Pat. Nos. 6,989,452, 7,087,600, and 7,129,261, and in PCT Application Nos. PCT/US2002/17210, PCT/US2005/017804, PCT/US2006/37793, PCT/US2006/060050, PCT/US2006/060711, WO2006/110476, and in U.S. Patent Application No. 60/891,028, all of which are incorporated herein by reference in their entirety. For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Affinity reagents can also be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, iodine131, indium111, yttrium90 and lutetium177. Methods for preparing radioimmunoconjugates are established in the art. Examples of radioimmunoconjugates are commercially available, including Zevalin® (IDEC Pharmaceuticals) and Bexxar® (Corixa Pharmaceuticals), and similar methods can be used to prepare radioimmunoconjugates using the antibodies of the invention.

Affinity reagents can also be conjugated to a phthalocyanine dye referred to hereafter as phthalocyanineconjugates. Examples of phthalocyanine dyes that can be conjugated to antibodies for use diagnostically or therapeutically include, but are not limited to, IR700. Methods for preparing phthalocyanineconjugates are described, for example, in Mitsunaga M, Ogawa M, Kosaka N, Rosenblum L T, Choyke P L and Kobayashi H (2011) Nat. Med. 2011 Nov. 6. doi: 10.1038/nm.2554.

The conjugates can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-γ; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Senter P. D. (2009) Curr. Opin. Chem. Biol. 13(3):235-244; Kovtun et al. (2010) Cancer Res. 70(6):2528-2537.

Techniques for conjugating such therapeutic moieties to antibodies are well known, see, e.g. Amon et al., “Monoclonal Antibodies For Immunotargeting Of Drugs In Cancer Therapy” in Monoclonal Antibodies And Cancer Therapy, Reisfeld et al. (eds.), pp. 243-56 (Alan R. Liss, Inc. 1985); Hellstrom et al., “Antibodies For Drug Delivery,” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); “Analysis, Results, And Future Prospective Of The Therapeutic Use Of Radiolabelled Antibody In Cancer Therapy” in Monoclonal Antibodies For Cancer Detection And Therapy, Baldwin et al. (eds.), pp. 303-16 (Academic Press 1985), and Thorpe et al., Immunol. Rev., 62:119-58 (1982).

Alternatively, an antibody can be conjugated to a second antibody to form an antibody heteroconjugate as described by Segal in U.S. Pat. No. 4,676,980.

An antibody with or without a therapeutic moiety conjugated to it can be used as a therapeutic that is administered alone or in combination with cytotoxic factor(s) and/or cytokine(s).

The invention also provides for fully human or humanised antibodies that induce antibody-directed cell-mediated cytotoxicity (ADCC). A fully human antibody is one in which the protein sequences are encoded by naturally occurring human immunoglobulin sequences, either from isolated antibody-producing human B-lymphocytes, or from transgenic murine B-lymphocytes of mice in which the murine immunoglobulin coding chromosomal regions have been replaced by orthologous human sequences. Transgenic antibodies of the latter type include, but are not restricted to, HuMab (Medarex, Inc, CA) and XenoMouse (Abgenix Inc., CA). A humanised antibody is one in which the constant region of a non-human antibody molecule of appropriate antigen specificity, is replaced by the constant region of a human antibody, preferably of the IgG subtype, with appropriate effector functions (Morrison et al., 1984, Proc. Natl. Acad. Sci. 81:851-855; Neuberger et al., 1984, Nature 312:604-608; Takeda et al., 1985, Nature 314:452-454). Appropriate effector functions include ADCC, which is a natural process by which fully-human antibodies or humanized antibodies, when bound to targets on the surface of cancer cells, switch on the cell killing properties of lymphocytes that are part of the normal immune system. These active lymphocytes, called Natural Killer (NK) cells, use a cytotoxic process to destroy living cells to which the antibodies are bound. ADCC activity may be detected and quantified by measuring release of Europium (Eu³⁺) from Eu³⁺ labelled, living cells in the presence of an antigen-specific antibody and peripheral blood mononuclear cells extracted from an immunocompetent, living human subject. The ADCC process is described in detail in Janeway Jr. C. A. et al., Immunobiology, 5th ed., 2001, Garland Publishing, ISBN 0-8153-3642-X; Pier G. B. et al., Immunology, Infection, and Immunity, 2004, p246-5; Albanell J. et al., Advances in Experimental Medicine and Biology, 2003, 532:p2153-68 and Weng, W.-K. et al., Journal of Clinical Oncology, 2003, 21:p 3940-3947. Suitable methods for the detection and quantification of ADCC can be found in Blomberg et al., Journal of Immunological Methods. 1986, 86:p225-9; Blomberg et al., Journal of Immunological Methods. 1986, 21;92:p117-23 and Patel & Boyd, Journal of Immunological Methods. 1995, 184:p29-38.

ADCC typically involves activation of NK cells and is dependent on the recognition of antibody-coated cells by Fc receptors on the surface of the NK cell. The Fc receptors recognize the Fc (crystalline) portion of antibodies such as IgG, bound specifically to the surface of a target cell. The Fc receptor that triggers activation of the NK cell is called CD16 or FcγRIIIa. Once the FcγRIIIa receptor is bound to the IgG Fc, the NK cell releases cytokines such as IFN-γ, and cytotoxic granules containing perforin and granzymes that enter the target cell and promote cell death by triggering apoptosis.

The induction of antibody-dependent cellular cytotoxicity (ADCC) by an antibody can be enhanced by modifications that alter interactions between the antibody constant region (Fc) and various receptors that are present on the surface of cells of the immune system. Such modifications include the reduction or absence of alpha-1,6-linked fucose moieties in the complex oligosaccharide chains that are normally added to the Fc of antibodies during natural or recombinant synthesis in mammalian cells. In a preferred embodiment, non-fucosylated affinity reagents such as antibodies or fragments thereof are produced for the purpose of enhancing their ability to induce the ADCC response.

Techniques for reducing or ablating alpha 1,6-linked fucose moieties in the oligosaccharide chains of the Fc are well established. In one example, the recombinant antibody is synthesized in a cell line that is impaired in its ability to add fucose in an alpha 1,6 linkage to the innermost N-acetylglucosamine of the N-linked biantennary complex-type Fc oligosaccharides. Such cell lines include, but are not limited to, the rat hybridoma YB2/0, which expresses a reduced level of the alpha 1,6-fucosyltransferase gene, FUT8. Preferably, the antibody is synthesized in a cell line that is incapable of adding alpha 1,6-linked fucosyl moieties to complex oligosaccharide chains, due to the deletion of both copies of the FUT8 gene. Such cell lines include, but are not limited to, FUT8−/− CHO/DG44 cell lines. Techniques for synthesizing partially fucosylated, or non-fucosylated antibodies and affinity reagents are described in Shinkawa et al., J. Biol. Chem. 278:3466-34735 (2003); Yamane-Ohnuki et al., Biotechnology and Bioengineering 87: 614-22 (2004) and in WO00/61739 A1, WO02/31140 A1 and WO03/085107 A1. In a second example, the fucosylation of a recombinant antibody is reduced or abolished by synthesis in a cell line that has been genetically engineered to overexpress a glycoprotein-modifying glycosyl transferase at a level that maximizes the production of complex N-linked oligosaccharides carrying bisecting N-acetylglucosamine. For example, the antibody is synthesized in a Chinese Hamster Ovary cell line expressing the enzyme N-acetyl glucosamine transferase III (GnT III). Cell lines stably transfected with suitable glycoprotein-modifying glycosyl transferases, and methods of synthesizing antibodies using these cells are described in WO99/54342.

A non-fucosylated antibody or affinity reagent can be used as a therapeutic that is administered alone or in combination with cytotoxic factor(s) and/or cytokine(s).

In a further modification, the amino acid sequences of the antibody Fc are altered in a way that enhances ADCC activation, without affecting ligand affinity. Examples of such modifications are described in Lazar et al., Proceedings of the National Academy of Sciences 2006, 103: p4005-4010; WO03/074679 and WO2007/039818. In these examples, substitution of amino acids in the antibody Fc, such as aspartate for serine at position 239, and isoleucine for glutamate at position 332, altered the binding affinity of an antibody for Fc receptors, leading to an increase in ADCC activation.

An antibody reagent with enhanced ADCC activation due to amino acid substitutions can be used as a therapeutic that is administered alone or in combination with cytotoxic factor(s) and/or cytokine(s).

The invention also provides for bispecific molecules comprising at least one first binding specificity for a first target epitope (i.e. one of the proteins of the invention) and a second binding specificity for a second target epitope. The second target epitope maybe present on the same target protein as that bound by the first binding specificity; or the second target epitope may be present of a different target protein to that bound by the first protein to that bound by the first binding specificity. The second target epitope may be present on the same cell as the first target epitope (i.e. one of the proteins of the invention); or the second target epitope may be present on a target which is not displayed by the cell which displays the first target epitope. As used herein, the term ‘binding specificity’ refers to a moiety comprising at least one antibody variable domain.

These bispecific molecules target one of the proteins of the invention expressing cells to CD3 expressing effector cells (e.g. CD3 expressing cytotoxic T cells) and trigger CD3-mediated effector cell activities, such as T cell clonal expansion and T cell cytotoxicity. The bispecific antibodies of the invention may have a total of either two or three antibody variable domains, wherein first portion of the bispecific antibody is capable of recruiting the activity of a human immune effector cell by specifically binding to an effector antigen located on the human immune effector cell, in which the effector antigen is the human CD3 antigen, said first portion consisting of one antibody variable domain, and a second portion of the bispecific antibody is capable of specifically binding to a target antigen other than the effector antigen e.g. one of the proteins of the invention, said target antigen being located on a target cell other than said human immune effector cell, and said second portion comprising one or two antibody variable domains.

### Therapeutic Use of the Proteins of the Invention

The invention provides for treatment or prevention of various diseases and disorders by administration of a therapeutic compound. Such compounds include but are not limited to: OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, analogs thereof, related polypeptides and derivatives (including fragments) thereof; antibodies (or other affinity reagents) to the foregoing; nucleic acids encoding an OGTA, analogs of the latter or a related polypeptides and fragments thereof; antisense nucleic acids to a gene encoding an OGTA, analogs of the latter or a related polypeptides and fragments thereof; and modulator (e.g., agonists and antagonists) of a gene encoding an OGTA or a related polypeptide. An important feature of the present invention is the identification of genes encoding OGTA(s) involved in a relevant cancer. B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma can be treated (e.g. to ameliorate symptoms or to retard onset or progression) or prevented by administration of a therapeutic compound that reduces function or expression of an OGTA in the serum or tissue of subjects having said cancer.

In one embodiment, one or more antibodies (or other affinity reagents) each specifically binding to a relevant OGTA are administered alone or in combination with one or more additional therapeutic compounds or treatments.

In one embodiment a biological product such as an antibody (or other affinity reagent) is allogeneic to the subject to which it is administered. In another embodiment, a human OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 or a related polypeptide, a nucleotide sequence encoding any of the aforementioned moieties, or an antibody (or other affinity reagent) to a human OGTA or a related polypeptide, is administered to a human subject for therapy (e.g. to ameliorate symptoms or to retard onset or progression) or prophylaxis.

Without being limited by theory, it is conceived that the therapeutic activity of antibodies (or other affinity reagents) which specifically bind to the proteins of the invention may be achieved through the phenomenon of Antibody—Dependent Cell-mediated Cytotoxicity (ADCC) (see e.g. Janeway Jr. C. A. et al., Immunobiology, 5th ed., 2001, Garland Publishing, ISBN 0-8153-3642-X; Pier G. B. et al., Immunology, Infection, and Immunity, 2004, p246-5; Albanell J. et al., Advances in Experimental Medicine and Biology, 2003, 532:p2153-68 and Weng, W.-K. et al., Journal of Clinical Oncology, 2003, 21:p 3940-3947).

Treatment and Prevention and/or diagnosis of B-cell Non-Hod kin's Lymphoma Breast Cancer, Cervical Cancer, Colorectal Cancer, Gastric Cancer, Glioblastoma, Hepatocellular Carcinoma, Lung Cancer, Lymphoid Leukaemia (Particularly Acute T-cell Leukaemia and Chronic Lymphocytic Leukaemia), Melanoma, Neuroblastoma, Osteosarcoma, Ovarian Cancer, Pancreatic Cancer, Prostate Cancer, Renal Cell Cancer and Retinoblastoma

Relevant cancers are treated or prevented by administration to a subject, suspected of having or known to have or to be at risk of developing said cancer, of a compound that modulates (i.e., increases or decreases) the level or activity (i.e., function or expression) of an OGTA according to the invention that is differentially present in the serum or tissue of subjects having a relevant cancer listed herein compared with serum or tissue of subjects free from said cancer.

In one embodiment, the cancer/cancers is/are treated or prevented by administering to a subject suspected having or known to have or to be at risk of developing a relevant cancer a compound that upregulates (i.e., increases) the level or activity (i.e., function or expression) of an OGTA according to the invention that are decreased in the serum or tissue of subjects having a relevant cancer. Examples of such a compound include, but are not limited to, OGTA antisense oligonucleotides, ribozymes, antibodies (or other affinity reagents) directed against the proteins of the invention, and compounds that inhibit the enzymatic activity of the proteins of the invention. Other useful compounds e.g. OGTA antagonists and small molecule OGTA antagonists, can be identified using in vitro assays.

A relevant cancer/cancers is/are also treated or prevented by administration to a subject suspected of having or known to have said cancer or to be at risk of developing said cancer of a compound that downregulates the level or activity (i.e. function) of an OGTA that are increased in the serum or tissue of subjects having said cancer. Examples of such a compound include but are not limited to: OGTAs according to the invention, OGTA fragments and OGTA-related polypeptides; nucleic acids encoding an OGTA, an OGTA fragment and an OGTA-related polypeptide (e.g. for use in gene therapy); and, for those OGTA or OGTA-related polypeptides with enzymatic activity, compounds or molecules known to modulate that enzymatic activity. Other compounds that can be used, e.g. OGTA agonists, can be identified using in in vitro assays.

In one embodiment, therapy or prophylaxis is tailored to the needs of an individual subject. Thus, in specific embodiments, compounds that promote the level or function of an OGTA according to the invention are therapeutically or prophylactically administered to a subject suspected of having or known to have a relevant cancer, in whom the levels or functions of an OGTA according to the invention are absent or are decreased relative to a control or normal reference range. In further embodiments, compounds that promote the level or function of an OGTA according to the invention are therapeutically or prophylactically administered to a subject suspected of having or known to have a relevant cancer in whom the levels or functions of an OGTA according to the invention are increased relative to a control or to a reference range. In further embodiments, compounds are employed that decrease the level or function of an OGTA according to the invention, for example in subjects in whom said levels or functions are increased relative to a control or to a reference range.

In further embodiments, compounds that decrease the level or function of OGTA according to the invention are therapeutically or prophylactically administered to a subject suspected of having or known to have a relevant cancer in whom said levels or functions are increased relative to a control or to a reference range.

In further embodiments, compounds that decrease the level or function of an OGTA according to the invention are therapeutically or prophylactically administered to a subject suspected of having or known to have a relevant cancer in whom said levels or functions are decreased relative to a control or to a reference range.

The change in OGTA function or level due to the administration of such compounds can be readily detected, e.g., by obtaining a sample (e.g., blood or urine) and assaying in vitro the levels or activities of an OGTA according to the invention, or the levels of mRNAs encoding same, or any combination of the foregoing. Such assays can be performed before and after the administration of the compound as described herein.

The compounds of the invention include but are not limited to any compound, e.g., a small organic molecule, protein, peptide, antibody (or other affinity reagent), nucleic acid, etc. that restores the relevant OGTAs profile towards normal. The compounds of the invention may be given in combination with any other chemotherapy drugs.

In accordance with the present invention, test samples of lymphoid, breast, cervical, colorectal, gastric, brain, liver, lung, skin, neuronal, osteoblast, ovarian, pancreatic, prostate, kidney or eye tissue, serum, plasma or urine obtained from a subject suspected of having or known to have a relevant cancer can be used for diagnosis or monitoring. In one embodiment, a change in the abundance of an OGTA according to the invention in a test sample relative to a control sample (from a subject or subjects free from said cancer or a previously determined reference range indicates the presence of the relevant cancer. In another embodiment, the relative abundance of an OGTA according to the invention in a test sample compared to a control sample or a previously determined reference range indicates a subtype of the relevant cancer (e.g. pre-T-cell or mature T-cell acute lymphocytic leukaemia, diffuse large B-cell lymphoma, inflammatory breast cancer, squamous cell cervical carcinoma, T-cell chronic lymphocytic leukaemia, familial or sporadic colorectal cancer, gastrointestinal stromal tumours, fibrolamellar hepatocellular carcinoma, squamous cell lung carcinoma, ganglioneuroblastoma or familial neuroblastoma, parosteal or periosteal osteosarcoma, malignant papillary serous adenocarcinoma, endocrine tumours of the pancreas or bilateral, multifocal retinoblastoma or unilateral, unifocal retinoblastoma). In yet another embodiment, the relative abundance of OGTA(s) in a test sample relative to a control sample or a previously determined reference range indicates the degree or severity of the relevant cancer (e.g., the likelihood for metastasis). In any of the aforesaid methods, detection of OGTA(s) may optionally be combined with detection of one or more of additional biomarkers for a relevant cancer. Any suitable method in the art can be employed to measure the level of OGTA(s), including but not limited to the Preferred Technologies described in Examples 1 and 2 described herein, kinase assays, immunoassays to detect and/or visualize OGTA(s) (e.g., Western blot, immunoprecipitation followed by sodium dodecyl sulfate polyacrylamide gel electrophoresis, immunocytochemistry, etc.). In a further embodiment, a change in the abundance of mRNA encoding an OGTA according to the invention in a test sample relative to a control sample or a previously determined reference range indicates the presence of a relevant cancer. Any suitable hybridization assay can be used to detect expression of an OGTA according to the invention by detecting and/or visualizing mRNA encoding an OGTA according to the invention (e.g., Northern assays, dot blots, in situ hybridization, etc.).

In another embodiment of the invention, labeled antibodies (or other affinity reagents), derivatives and analogs thereof, which specifically bind to an OTGA according to the invention can be used for diagnostic purposes to detect, diagnose, or monitor a relevant cancer. For example a relevant cancer is detected in an animal, such as in a mammal and particularly in a human.

### Screening Assays

The invention provides methods for identifying agents (e.g., candidate compounds or test compounds) that bind to an OTGA according to the invention or have a stimulatory or inhibitory effect on the expression or activity of an OGTA according to the invention. The invention also provides methods of identifying agents, candidate compounds or test compounds that bind to an OGTA according to the invention or a related polypeptide or an fusion protein or have a stimulatory or inhibitory effect on the expression or activity of any of the aforementioned moieties. Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233, each of which is incorporated herein in its entirety by reference.

Libraries of compounds may be presented, e.g., presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310), each of which is incorporated herein in its entirety by reference.

In one embodiment, agents that interact with (i.e., bind to) OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, a fragment (e.g. a functionally active fragment) thereof, a related polypeptide, a fragment of a related polypeptide, or an OGTA fusion protein are identified in a cell-based assay system.

In accordance with this embodiment, cells expressing OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, a fragment thereof, a related polypeptide, a fragment of a related polypeptide, or an OGTA fusion protein are contacted with a candidate compound or a control compound and the ability of the candidate compound to interact with an OGTA according to the invention is determined. If desired, this assay may be used to screen a plurality (e.g. a library) of candidate compounds. The cell, for example, can be of prokaryotic origin (e.g., E. coli) or eukaryotic origin (e.g., yeast or mammalian). Further, the cells can express OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, a fragment thereof, a related polypeptide, a fragment of a related polypeptide, or an OGTA fusion protein endogenously or be genetically engineered to express one or more of the same. In certain instances, OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, a fragment thereof, a related polypeptide, a fragment of a related polypeptide, or an OGTA fusion protein or the candidate compound is labeled, for example with a radioactive label (such as ³²P, ³⁵S, and ¹²⁵I) or a fluorescent label (such as fluorescein isothiocyanate, rhodamine, phycoerythrin, phycocyanin, allophycocyanin, o-phthaldehyde or fluorescamine) to enable detection of an interaction between a relevant OGTA according to the invention and a candidate compound. The ability of the candidate compound to interact directly or indirectly with OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, a fragment thereof, a related polypeptide, a fragment of a related polypeptide, or an OGTA fusion protein can be determined by methods known to those of skill in the art. For example, the interaction between a candidate compound and OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, a related polypeptide, a fragment of a related polypeptide, or an OGTA fusion protein can be determined by flow cytometry, a scintillation assay, immunoprecipitation or western blot analysis.

In another embodiment, agents that interact with (i.e., bind to) OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, a fragment (e.g., a functionally active fragment) thereof, a related polypeptide, or an OGTA fusion protein are identified in a cell-free assay system. In accordance with this embodiment, a native or recombinant OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 or fragment thereof, or a native or recombinant related polypeptide or fragment thereof, or an OGTA fusion protein or fragment thereof, is contacted with a candidate compound or a control compound and the ability of the candidate compound to interact with OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 or the related polypeptide, or the fusion protein is determined. If desired, this assay may be used to screen a plurality (e.g. a library) of candidate compounds.

In one embodiment, OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, a fragment thereof, a related polypeptide, a fragment of a related polypeptide, or an OGTA fusion protein is first immobilized, by, for example, contacting the relevant entity with an immobilized antibody (or other affinity reagent) which specifically recognizes and binds it, or by contacting a purified preparation of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, a fragment thereof, a related polypeptide, a fragment of a related polypeptide, or an OGTA fusion protein with a surface designed to bind proteins.

OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, a fragment thereof, a related polypeptide, a fragment of a related polypeptide, or an OGTA fusion protein may be partially or completely purified (e.g., partially or completely free of other polypeptides) or part of a cell lysate. Further, OGTAs of the invention, a fragment thereof, a related polypeptide, a fragment of a related polypeptide may be a fusion protein comprising OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 or a biologically active portion thereof, or a related polypeptide and a domain such as glutathionine-S-transferase.

Alternatively, an OGTA according to the invention, a fragment fragment thereof, a related polypeptide, fragment of a related polypeptide or an OGTA fusion protein can be biotinylated using techniques well known to those of skill in the art (e.g., biotinylation kit, Pierce Chemicals; Rockford, Ill.). The ability of the candidate compound to interact with an OGTA accordintg to the invention, a fragment thereof, a related polypeptide, a fragment of a related polypeptide, or an OGTA fusion protein can be determined by methods known to those of skill in the art.

In another embodiment, a cell-based assay system is used to identify agents that bind to or modulate the activity of a protein, such as an enzyme, or a biologically active portion thereof, which is responsible for the production or degradation of an OGTA according to the inventionr is responsible for the post-translational modification of same. In a primary screen, a plurality (e.g., a library) of compounds are contacted with cells that naturally or recombinantly express: (i) OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, an isoform of any of the aforementioned, a homolog thereof, an a related polypeptide, an OGTA fusion protein, or a biologically active fragment of any of the foregoing; and (ii) a protein that is responsible for processing of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, isoform of any of the aformentioned, an OGTA homolog, a related polypeptide, an OGTA fusion protein, or fragment in order to identify compounds that modulate the production, degradation, or post-translational modification of the same. If desired, compounds identified in the primary screen can then be assayed in a secondary screen against cells naturally or recombinantly expressing a protein according to the invention. The ability of the candidate compound to modulate the production, degradation or post-translational modification of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, isoform, homolog, a related polypeptide, or OGTA fusion protein can be determined by methods known to those of skill in the art, including without limitation, flow cytometry, a scintillation assay, immunoprecipitation and western blot analysis.

In another embodiment, agents that competitively interact with (i.e., bind to) OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, a fragment thereof, a related polypeptide, a fragment of a related polypeptide, or an OGTA fusion protein are identified in a competitive binding assay.

In accordance with this embodiment, cells expressing an OGTA according to the invention, a fragment thereof, a related polypeptide, a fragment of a related polypeptide, or an OGTA fusion protein are contacted with a candidate compound and a compound known to interact with the same (any of the aforementioned); the ability of the candidate compound to preferentially interact with OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, a fragment thereof, a related polypeptide, fragment of a related polypeptide, or an OGTA fusion protein is then determined.

Alternatively, agents that preferentially interact with (i.e., bind to) an OGTA according to the invention, a fragment thereof, a related polypeptide or fragment of a related polypeptide are identified in a cell-free assay system by contacting OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, a fragment thereof, a related polypeptide, a fragment of a related polypeptide, or an OGTA fusion protein with a candidate compound and a compound known to interact with an OGTA according to the invention, a related polypeptide or the OGTA fusion protein.

As stated above, the ability of the candidate compound to interact with an OGTA according to the invention, a fragment thereof, a related polypeptide, a fragment of a related polypeptide, or an OGTA fusion protein can be determined by methods known to those of skill in the art. These assays, whether cell-based or cell-free, can be used to screen a plurality (e.g., a library) of candidate compounds.

In another embodiment, agents that modulate (i.e., upregulate or downregulate) the expression or activity of an OGTA according to the invention or a related polypeptide are identified by contacting cells (e.g., cells of prokaryotic origin or eukaryotic origin) expressing OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, or a related polypeptide with a candidate compound or a control compound (e.g., phosphate buffered saline (PBS)) and determining the expression of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, a related polypeptide, or an OGTA fusion protein, mRNA encoding an OGTA according to theinvention, or mRNA encoding OGTA according to the invention or a related polypeptide.

The level of expression of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, a related polypeptide, mRNA encoding an OGTA according to the invention, or mRNA encoding an OGTA related polypeptide in the presence of the candidate compound is compared to the level of expression in the absence of the candidate compound (e.g., in the presence of a control compound). The candidate compound can then be identified as a modulator of the expression of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, or a related polypeptide based on this comparison. For example, when expression of an OGTA according to the invention or mRNA is significantly greater in the presence of the candidate compound than in its absence, the candidate compound is identified as a stimulator of expression of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 or mRNA. Alternatively, when expression of OGTA according to the invention or mRNA is significantly less in the presence of the candidate compound than in its absence, the candidate compound is identified as an inhibitor of the expression of OGTA according to the invention or mRNA. The level of expression of OGTA according to the invention or the mRNA that encodes it can be determined by methods known to those of skill in the art. For example, mRNA expression can be assessed by Northern blot analysis or RT-PCR, and protein levels can be assessed by western blot analysis.

In another embodiment, agents that modulate the activity of an OGTA according to the invention or a related polypeptide are identified by contacting a preparation containing OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 or a related polypeptide or cells (e.g., prokaryotic or eukaryotic cells) expressing any of the aforementioned with a test compound or a control compound and determining the ability of the test compound to modulate (e.g., stimulate or inhibit) the activity of an OGTA according to the invention or a related polypeptide. The activity of OGTA according to the invention or a related polypeptide can be assessed by detecting induction of a cellular signal transduction pathway of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 or a related polypeptide (e.g., intracellular Ca²⁺, diacylglycerol, IP3, etc.), detecting catalytic or enzymatic activity of the target on a suitable substrate, detecting the induction of a reporter gene (e.g., a regulatory element that is responsive to to an OGTA according to the invention or a related polypeptide and is operably linked to a nucleic acid encoding a detectable marker, e.g., luciferase), or detecting a cellular response, for example, cellular differentiation, or cell proliferation. Based on the present description, techniques known to those of skill in the art can be used for measuring these activities (see, e.g., U.S. Pat. No. 5,401,639, which is incorporated herein by reference).

The candidate compound can then be identified as a modulator of the activity of an OGTA according to the invention or a related polypeptide by comparing the effects of the candidate compound to the control compound. Suitable control compounds include phosphate buffered saline (PBS) and normal saline (NS).

In another embodiment, agents that modulate (i.e., upregulate or downregulate) the expression, activity or both the expression and activity of an OGTA according to the invention or a related polypeptide are identified in an animal model. Examples of suitable animals include, but are not limited to, mice, rats, rabbits, monkeys, guinea pigs, dogs and cats. For example, the animal used represent a model of a relevant cancer (e.g. xenografts of acute T-cell leukaemia cell lines such as HSB-2 in SCID mice, Morland et al, Cell Biophys. 1994;24-25:315-29; xenografts of B-cell non-Hodgkin's lymphoma cell lines such as SU-DHL-4 and OCI-Ly8 in SCID mice, Schmidt-Wolf et al, J Exp Med. 1991 Jul. 1; 174(1):139-49; xenografts of breast cancer cell lines such as MCF-7 (Ozzello L, Sordat M., Eur J. Cancer. 1980; 16:553-559) and MCF10AT (Miller et al., J Natl Cancer Inst. 1993; 85:1725-1732) in nude or SCID mice; xenografts of cervical cancer cell lines such as CaSki in nude mice; xenografts of chronic lymphocytic leukaemia cell lines such as WSU-CLL in SCID mice, Mohammad et al, Leukaemia. 1996 January;10(1):130-7; xenografts of human colorectal cancer cell lines such as MDA-MB-345 in oestrogen-deprived SCID mice, Eccles et al. 1994 Cell Biophysics 24/25, 279; xenografts of gastric cell lines such as AZ-521 in nude mice; xenografts of glioblastoma cell lines such as U87MG in nude mice, Abernathey et al., Neurosurgery 1988 May; 22(5):877-81; xenografts of hepatocellular carcinoma cell lines such as MHCC97 in nude mice, Tian et al., Br J 5 Cancer 1999 November; 81(5):814-21; xenografts of non small cell lung cancer cell lines such as A549 and H460 and xenografts of small cell lung cancer cell lines such as NC1—H345; xenografts of melanoma cell lines such as MV3 in nude mice, van Muijen et al, Int J Cancer 1991 Apr. 22; 48(1):85-91; xenografts of neuroblastoma cell lines such as SK-N—SH in nude mice, Helson et al, Cancer Res. 1975 September; 35(9):2594-9; xenografts of human osteosarcoma cell lines such as Hu09 in nude mice, Kimura et al., Clin Exp Metastasis 2002; 19(6):477-85; xenografts of ovarian cancer cell lines such as IGROV1 in nude mice, Benard et al, Cancer Res. 1985 October; 45(10):4970-9; xenografts of pancreatic cancer cell lines such as MIA PaCa-2 in nude mice, Marincola et al., J Surg Res 1989 December; 47(6):520-9; xenografts of prostate cancer cell lines such as CWR-22 in nude mice, Pretlow et al, J Natl Cancer Inst. 1993 Mar. 3; 85(5):394-8; xenografts of renal cell cancer cell lines such as LABAZ1 in immune compromised mice, Zisman et al, Cancer Research 63, 4952-4959, Aug. 15, 2003, or xenografts of retinoblastoma cell lines such as Y79.). These can be utilized to test compounds that modulate levels of an OGTA according to the invention, since the pathology exhibited in these models is similar to that of a relevant cancer. In accordance with this embodiment, the test compound or a control compound is administered (e.g., orally, rectally or parenterally such as intraperitoneally or intravenously) to a suitable animal and the effect on the expression, activity or both expression and activity of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 or a related polypeptide is determined. Changes in the expression of the same can be assessed by the methods outlined above.

In yet another embodiment, OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 or a related polypeptide is used as a “bait protein” in a two-hybrid assay or three hybrid assay to identify other proteins that bind to or interact with an OGTA according to the invention or a related polypeptide (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300). As those skilled in the art will appreciate, such binding proteins are also likely to be involved in the propagation of signals by OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 as, for example, upstream or downstream elements of a signaling pathway involving same.

This invention provides novel agents identified by screening assays and uses thereof for treatments as described herein. In addition, the invention also provides the use of an agent which interacts with, or modulates the activity of, an OGTA according to the invention in the manufacture of a medicament for the treatment and/or diagnosis of a relevant cancer.

### Vaccine Therapy

Another aspect of the invention is an immunogenic composition, suitably a vaccine composition, comprising OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 or an epitope containing fragment thereof, or nucleic acid encoding OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 or a fragment thereof optionally together with an immunostimulant.

There is also provided a method of raising an immune response which comprises administering to a subject such compositions and a method for treating or preventing a relevant cancer which comprises administering to a subject in need thereof a therapeutically effective amount of such compositions and such compositions for use in preventing or treating a relevant cancer.

Thus, OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 and OGTA271 may be useful as antigenic material, and may be used in the production of vaccines for treatment or prophylaxis of B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma. Such material can be “antigenic” and/or “immunogenic”. Generally, “antigenic” is taken to mean that the protein is capable of being used to raise antibodies (or other affinity reagents) or indeed is capable of inducing an antibody response in a subject or experimental animal. “Immunogenic” is taken to mean that the protein is capable of eliciting a protective immune response in a subject or experimental animal. Thus, in the latter case, the protein may be capable of not only generating an antibody response but, in addition, non-antibody based immune responses. “Immunogenic” also embraces whether the protein may elicit an immune-like response in an in-vitro setting e.g. a T-cell proliferation assay.

The immune response may require appropriate formulation or presentation of the immunogen and may require the presence of one or more excipients such as one or more adjuvant components. Nevertheless the invention extends to such composition which may form a key component of a final vaccine.

The skilled person will appreciate that homologues or derivatives of OGTA according to the invention will also find use as antigenic/immunogenic material and for other applications according to the invention. Thus, for instance proteins which include one or more additions, deletions, substitutions or the like are encompassed by the present invention. In addition, it may be possible to replace one amino acid with another of similar “type”. For instance, replacing one hydrophobic amino acid with another. One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention.

In the case of homologues and derivatives, the degree of identity with a protein as described herein is less important than that the homologue or derivative should retain its antigenicity and/or immunogenicity. However, suitably, homologues or derivatives having at least 60% similarity (as discussed above) or identity with the proteins or polypeptides described herein are provided. Preferably, homologues or derivatives having at least 70% similarity or identity, more preferably at least 80% similarity or identity are provided. Most preferably, homologues or derivatives having at least 90% or even 95% similarity or identity are provided.

In an alternative approach, the homologues or derivatives could be fusion proteins, incorporating moieties which render purification easier, for example by effectively tagging the desired protein or polypeptide. It may be necessary to remove the “tag” or it may be the case that the fusion protein itself retains sufficient antigenicity to be useful.

It is well known that it is possible to screen an antigenic protein or polypeptide to identify epitopic regions, i.e. those regions which are responsible for the protein or polypeptide's antigenicity or immunogenicity. Methods well known to the skilled person can be used to test fragments and/or homologues and/or derivatives for antigenicity. Thus, the fragments of the present invention should include one or more such epitopic regions or be sufficiently similar to such regions to retain their antigenic/immunogenic properties. Thus, for fragments according to the present invention the degree of identity is perhaps irrelevant, since they may be 100% identical to a particular part of a protein or polypeptide, homologue or derivative as described herein. The key issue, once again, is that the fragment retains the antigenic/immunogenic properties of the protein from which it is derived.

What is important for homologues, derivatives and fragments is that they possess at least a degree of the antigenicity/immunogenicity of the protein or polypeptide from which they are derived. Thus, in an additional aspect of the invention, there is provided antigenic/or immunogenic fragments of OGTAs according to the invention, or of homologues or derivatives thereof.

OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 and OGTA271, or antigenic fragments thereof, can be provided alone, as a purified or isolated preparation. They may be provided as part of a mixture with one or more other proteins of the invention, or antigenic fragments thereof. In a further aspect, therefore, the invention provides an antigen composition comprising an OGTA according to the invention and/or one or more antigenic fragments thereof. Such a composition can be used for the detection and/or diagnosis of a relevant cancer.

The antigenic OGTA according to the invention, antigenic fragment thereof or antigen composition of the invention can be used to induce an immune response against a relevant cancer.

In one aspect, the present invention provides a method of detecting and/or diagnosing a relevant cancer which comprises:

bringing into contact with a sample to be tested an antigenic OGTA according to the invention (including one or more of same), or an antigenic fragment thereof, or an antigen composition of the invention; and detecting the presence of antibodies (or other affinity reagents) to a relevant cancer.

In particular, the protein, antigenic fragment thereof or antigen composition of the present invention can be used to detect IgA, IgM or IgG antibodies. Suitably, the sample to be tested will be a biological sample, e.g. a sample of blood, tissue or saliva.

In a further aspect, the invention provides the use of an antigenic OGTA according to the invention, an antigenic fragment thereof or an antigen composition of the invention in medicine.

In a further aspect, the present invention provides a composition capable of eliciting an immune response in a subject, which composition comprises OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, an antigenic fragment thereof, or an antigen composition of the invention. Suitably, the composition will be a vaccine composition, optionally comprising one or more suitable adjuvants. Such a vaccine composition may be either a prophylactic or therapeutic vaccine composition.

Vaccine compositions according to the invention may be either a prophylactic or therapeutic vaccine composition.

The vaccine compositions of the invention can include one or more adjuvants (immunostimulants). Examples well-known in the art include inorganic gels, such as aluminium hydroxide, and water-in-oil emulsions, such as incomplete Freund's adjuvant. Other useful adjuvants will be well known to the skilled person.

Such preparations may include other vehicles.

In another embodiment, a preparation of oligonucleotides comprising 10 or more consecutive nucleotides complementary to a nucleotide sequence encoding an OGTA or an OGTA peptide fragments is used as vaccines for the treatment of a relevant cancer. Such preparations may include adjuvants or other vehicles.

Suitable adjuvants for use in vaccine compositions for the treatment of cancer include: 3De-O-acylated monophosphoryl lipid A (known as 3D-MPL or simply MPL see WO92/116556), a saponin, for example QS21 or QS7, and TLR4 agonists such as a CpG containing molecule, for example as disclosed in WO95/26204.

The adjuvants employed may be a combination of components, for example MPL and QS21 or MPL, QS21 and a CpG containing moiety.

Adjuvants may be formulated as oil-in-water emulsions or liposomal formulations.

In yet further aspects, the present invention provides:

(a) the use of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, an antigenic fragment thereof, or an antigen composition of the invention in the preparation of an immunogenic composition, preferably a vaccine;

(b) the use of such an immunogenic composition in inducing an immune response in a subject; and

(c) a method for the treatment or prophylaxis of B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma in a subject, or of vaccinating a subject against said cancer(s) which comprises the step of administering to the subject an effective amount of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271, at least one antigenic fragment thereof or an antigen composition of the invention, such as a vaccine.

In a specific embodiment, a preparation of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 or peptide fragments thereof is used as a vaccine for the treatment of a relevant cancer. Such preparations may include adjuvants or other vehicles.

The invention also extends to use of a vaccine as described herein for use in prophylaxis or treatment of an appropriate cancer as described herein.

In another embodiment, a preparation of oligonucleotides comprising 10 or more consecutive nucleotides complementary to a nucleotide sequence encoding OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 and OGTA271, antigenic fragments thereof or can be provided as a kit for use in the in vitro detection and/or diagnosis of a relevant cancer. Thus, in a still further aspect, the present invention provides a kit for use in the detection and/or diagnosis of a relevant cancer, which kit comprises an antigenic OTGA according to the invention, an antigenic fragment thereof or an antigenic composition of the present invention.

Identification of Compounds that Inhibit the Proteins of the Invention to Treat B-cell Non-Hodgkin's Lymphoma, Breast Cancer, Cervical Cancer, Colorectal Cancer, Gastric Cancer, Glioblastoma, Hepatocellular Carcinoma, Lung Cancer, Lymphoid Leukaemia (Particularly Acute T-cell Leukaemia and Chronic Lymphocytic Leukaemia), Melanoma, Neuroblastoma, Osteosarcoma, Ovarian Cancer, Pancreatic Cancer, Prostate Cancer, Renal Cell Cancer and Retinoblastoma

In one embodiment of the invention, B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma is treated or prevented by administration of a compound that antagonizes (inhibits) the level(s) and/or function(s) of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 which are elevated in the serum or tissue of subjects having B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma as compared with serum or tissue of subjects free from B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma.

Compounds useful for this purpose include but are not limited to anti-OGTA002, anti-OGTA009, anti-OGTA016, anti-OGTA028, anti-OGTA037, anti-OGTA041, anti-OGTA053, anti-OGTA054, anti-OGTA066, anti-OGTA072, anti-OGTA074, anti-OGTA076, anti-OGTA085, anti-OGTA087, anti-OGTA088, anti-OGTA089, anti-OGTA091, anti-OGTA098, anti-OGTA101, anti-OGTA104, anti-OGTA106, anti-OGTA112, anti-OGTA113, anti-OGTA119, anti-OGTA124, anti-OGTA126, anti-OGTA156, anti-OGTA159, anti-OGTA168, anti-OGTA169, anti-OGTA174, anti-OGTA176, anti-OGTA177, anti-OGTA197, anti-OGTA202, anti-OGTA203, anti-OGTA206, anti-OGTA213, anti-OGTA214. anti-OGTA216, anti-OGTA222, anti-OGTA236, anti-OGTA237, anti-OGTA247, anti-OGTA248, anti-OGTA249, anti-OGTA257 or anti-OGTA271 antibodies (or other affinity reagents, and fragments and derivatives containing the binding region thereof), OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 antisense or ribozyme nucleic acids, and nucleic acids encoding dysfunctional OGTAs that are used to “knockout” endogenous OGTA function by homologous recombination (see, e.g., Capecchi, 1989, Science 244:1288-1292). Other compounds that inhibit OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 function can be identified by use of known in vitro assays, e.g., assays for the ability of a test compound to inhibit binding of an OGTA according to the invention to another protein or a binding partner, or to inhibit a known OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 function. Such inhibition may, for example be assayed in vitro or in cell culture, but genetic assays may also be employed. The Preferred Technologies described in Examples 1 and 2 can also be used to detect levels of an OGTA according to the invention before and after the administration of the compound. Preferably, suitable in vitro or in vivo assays are utilized to determine the effect of a specific compound and whether its administration is indicated for treatment of the affected tissue.

In a specific embodiment, a compound that inhibits OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 function is administered therapeutically or prophylactically to a subject in whom an increased serum or tissue level or functional activity of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 (e.g., greater than the normal level or desired level) is detected as compared with serum or tissue of subjects free from B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma or a predetermined reference range.

Methods standard in the art can be employed to measure the increase in the level or function of an OGTA according to the invention, as outlined above.

In one embodiment OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 inhibitor compositions include small molecules, i.e. molecules of 1000 daltons or less. Such small molecules can be identified by the screening methods described herein.

### Assays for Therapeutic or Prophylactic Compounds

The present invention also provides assays for use in drug discovery in order to identify or verify the efficacy of compounds for treatment or prevention of a relevant cancer. Test compounds can be assayed for their ability to restore levels of an OGTA according to the invention in a subject having a relevant cancer towards levels found in subjects free from B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma (as appropriate) or to produce similar changes in experimental animal models of said cancers.

Compounds able to restore OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 levels in a subject having B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma towards levels found in subjects free from B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma or to produce similar changes in experimental animal models of said cancer can be used as lead compounds for further drug discovery, or used therapeutically.

OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 expression can be assayed by the Preferred Technologies described in Examples 1 and 2, immunoassays, gel electrophoresis followed by visualization, detection of OGTA activity, or any other method taught herein or known to those skilled in the art. Such assays can be used to screen candidate drugs, in clinical monitoring or in drug development, where abundance of OGTA(s) can serve as a surrogate marker for clinical disease.

In various specific embodiments, in vitro assays can be carried out with cells representative of cell types involved in a subject's disorder, to determine if a compound has a desired effect upon such cell types.

Compounds for use in therapy can be tested in suitable animal model systems prior to testing in humans, including but not limited to rats, mice, chicken, cows, monkeys, rabbits, etc. For in vivo testing, prior to administration to humans, any animal model system known in the art may be used. Examples of animal models of B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma include, but are not limited to, xenografts of acute T-cell leukaemia cell lines such as HSB-2 in SCID mice, Morland et al, Cell Biophys. 1994;24-25:315-29; xenografts of B-cell non-Hodgkin's lymphoma cell lines such as SU-DHL-4 and OCI-Ly8 in SCID mice, Schmidt-Wolf et al, J Exp Med. 1991 Jul. 1; 174(1):139-49; xenografts of breast cancer cell lines such as MCF-7 (Ozzello L, Sordat M., Eur J. Cancer. 1980; 16:553-559) and MCF10AT (Miller et al., J Natl Cancer Inst. 1993; 85:1725-1732) in nude or SCID mice; xenografts of cervical cancer cell lines such as CaSki in nude mice; xenografts of chronic lymphocytic leukaemia cell lines such as WSU-CLL in SCID mice, Mohammad et al, Leukaemia. 1996 January;10(1):130-7; xenografts of human colorectal cancer cell lines such as MDA-MB-345 in oestrogen-deprived SCID mice, Eccles et al. 1994 Cell Biophysics 24/25, 279; xenografts of gastric cell lines such as AZ-521 in nude mice; xenografts of glioblastoma cell lines such as U87MG in nude mice, Abernathey et al., Neurosurgery 1988 May; 22(5):877-81; xenografts of hepatocellular carcinoma cell lines such as MHCC97 in nude mice, Tian et al., Br J 5 Cancer 1999 November; 81(5):814-21; xenografts of non small cell lung cancer cell lines such as A549 and H460 and xenografts of small cell lung cancer cell lines such as NC1—H345; xenografts of melanoma cell lines such as MV3 in nude mice, van Muijen et al, Int J Cancer 1991 Apr. 22; 48(1):85-91; xenografts of neuroblastoma cell lines such as SK-N—SH in nude mice, Helson et al, Cancer Res. 1975 September; 35(9):2594-9; xenografts of human osteosarcoma cell lines such as Hu09 in nude mice, Kimura et al., Clin Exp Metastasis 2002; 19(6):477-85; xenografts of ovarian cancer cell lines such as IGROV1 in nude mice, Benard et al, Cancer Res. 1985 October; 45(10):4970-9; xenografts of pancreatic cancer cell lines such as MIA PaCa-2 in nude mice, Marincola et al., J Surg Res 1989 December; 47(6):520-9; xenografts of prostate cancer cell lines such as CWR-22 in nude mice, Pretlow et al, J Natl Cancer Inst. 1993 Mar. 3; 85(5):394-8; xenografts of renal cell cancer cell lines such as LABAZ1 in immune compromised mice, Zisman et al, Cancer Research 63, 4952-4959, Aug. 15, 2003, or xenografts of retinoblastoma cell lines such as Y79. These can be utilized to test compounds that modulate levels of an OGTA according to the invention, since the pathology exhibited in these models is similar to that of B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma.

It is also apparent to the skilled artisan that based upon the present disclosure, transgenic animals can be produced with “knock-out” mutations of the gene or genes encoding OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271. A “knock-out” mutation of a gene is a mutation that causes the mutated gene to not be expressed, or expressed in an aberrant form or at a low level, such that the activity associated with the gene product is nearly or entirely absent. Preferably, the transgenic animal is a mammal, more preferably, the transgenic animal is a mouse.

In one embodiment, test compounds that modulate the expression of OGTAs according to the invention are identified in non-human animals (e.g., mice, rats, monkeys, rabbits, and guinea pigs), preferably non-human animal models for B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma, expressing OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271. In accordance with this embodiment, a test compound or a control compound is administered to the animals, and the effect of the test compound on expression of an OGTA according to the invention is determined. A test compound that alters the expression of an OGTA according to the invention can be identified by comparing the level of an OGTA according to the invention (or mRNA(s) encoding the same) in an animal or group of animals treated with a test compound with the level of an OGTA according to the invention or mRNA(s) in an animal or group of animals treated with a control compound. Techniques known to those of skill in the art can be used to determine the mRNA and protein levels, for example, in situ hybridization. The animals may or may not be sacrificed to assay the effects of a test compound.

In another embodiment, test compounds that modulate the activity of an OGTA according to the invention or a biologically active portion thereof are identified in non-human animals (e.g., mice, rats, monkeys, rabbits, and guinea pigs), preferably non-human animal models for B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma, expressing an OGTA according to the invention. In accordance with this embodiment, a test compound or a control compound is administered to the animals, and the effect of a test compound on the activity of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 is determined. A test compound that alters the activity of an OGTA according to the invention can be identified by assaying animals treated with a control compound and animals treated with the test compound. The activity of OGTA according to the invention can be assessed by detecting induction of a cellular second messenger of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 (e.g., intracellular Ca²⁺, diacylglycerol, IP3, etc.), detecting catalytic or enzymatic activity of an OGTA according to the invention or binding partner thereof, detecting the induction of a reporter gene (e.g., a regulatory element that is responsive to OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 operably linked to a nucleic acid encoding a detectable marker, such as luciferase or green fluorescent protein), or detecting a cellular response (e.g., cellular differentiation or cell proliferation). Techniques known to those of skill in the art can be utilized to detect changes in the activity of an OGTA according to the invention (see, e.g., U.S. Pat. No. 5,401,639, which is incorporated herein by reference).

In yet another embodiment, test compounds that modulate the level or expression of an OGTA according to the invention are identified in human subjects having a relevant cancer, for example those having severe B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma.

In accordance with this embodiment, a test compound or a control compound is administered to the human subject, and the effect of a test compound on OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 expression is determined by analyzing the expression of an OGTA according to the invention or the mRNA encoding the same in a biological sample (e.g., serum, plasma, or urine). A test compound that alters the expression of an OGTA according to the invention can be identified by comparing the level of an OGTA according to the invention or mRNA encoding the same in a subject or group of subjects treated with a control compound to that in a subject or group of subjects treated with a test compound. Alternatively, alterations in the expression of an OGTA according to the invention can be identified by comparing the level of OGTA or mRNA encoding the same in a subject or group of subjects before and after the administration of a test compound. Techniques known to those of skill in the art can be used to obtain the biological sample and analyze the mRNA or protein expression. For example, the Preferred Technologies described in Examples 1 and 2 described herein can be used to assess changes in the level of OGTA.

In another embodiment, test compounds that modulate the activity of OGTAs are identified in human subjects having B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma, (preferably those with severe B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma). In this embodiment, a test compound or a control compound is administered to the human subject, and the effect of a test compound on the activity of the OGTA is determined. A test compound that alters the activity of the relevant OGTA can be identified by comparing biological samples from subjects treated with a control compound to samples from subjects treated with the test compound. Alternatively, alterations in the activity of a relevant OGTA can be identified by comparing the activity of a relevant OGTA in a subject or group of subjects before and after the administration of a test compound. The activity of a relevant OGTA can be assessed by detecting in a biological sample (e.g., serum, plasma, or urine) induction of a cellular signal transduction pathway of an OGTA according to the invention (e.g., intracellular Ca²⁺, diacylglycerol, IP3, etc.), catalytic or enzymatic activity of the OGTA or a binding partner thereof, or a cellular response, for example, cellular differentiation, or cell proliferation. Techniques known to those of skill in the art can be used to detect changes in the induction of a second messenger of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 or changes in a cellular response. For example, RT-PCR can be used to detect changes in the induction of a cellular second messenger.

In one embodiment, a test compound that changes the level or expression of an OGTA according to the invention towards levels detected in control subjects (e.g., humans free from B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma) is selected for further testing or therapeutic use. In another preferred embodiment, a test compound that changes the activity of the relevant OGTA towards the activity found in control subjects (e.g., humans free from B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma) is selected for further testing or therapeutic use.

In another embodiment, test compounds that reduce the severity of one or more symptoms associated with a relevant cancer are identified in human subjects having a relevant cancer, for example subjects with severe a relevant cancer. In accordance with this embodiment, a test compound or a control compound is administered to the subjects, and the effect of a test compound on one or more symptoms of a relevant cancer is determined. A test compound that reduces one or more symptoms can be identified by comparing the subjects treated with a control compound to the subjects treated with the test compound. Techniques known to physicians familiar with a relevant cancer can be used to determine whether a test compound reduces one or more symptoms associated with a relevant cancer. For example, a test compound that reduces tumour burden in a subject having a relevant cancer will be beneficial for subjects having B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma.

In a preferred embodiment, a test compound that reduces the severity of one or more symptoms associated with B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma in a human having B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma is selected for further testing or therapeutic use.

Therapeutic and Prophylactic Compositions and their Use

The invention provides methods of treatment (and prophylaxis) comprising administering to a subject an effective amount of a compound of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In a specific embodiment, a non-human mammal is the subject.

Formulations and methods of administration that can be employed when the compound comprises a nucleic acid are described above; additional appropriate formulations and routes of administration are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, or fibers. In one embodiment, administration can be by direct injection into lymphoid, breast, cervical, colorectal, gastric, brain, liver, lung, skin, neuronal, osteoblast, ovarian, pancreatic, prostate, kidney and eye tissue or at the site (or former site) of a malignant tumour or neoplastic or pre-neoplastic tissue.

In another embodiment, the compound can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compound can be delivered in a controlled release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, e.g. the breast, cervix, colon, stomach, brain, liver, lung, skin, bone, ovary, pancreas, prostate, kidney or eye, thus requiring only a fraction of the systemic dose (see, e.g., Goodson, in Medical Applications of Controlled Release, supra, vol. 2, pp. 115-138 (1984)).

Other controlled release systems are discussed in the review by Langer (1990, Science 249:1527-1533).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water may be a suitable carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In one embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. The amount of the compound of the invention which will be effective in the treatment of B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer or retinoblastoma can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight or more such as 10 or 100 mg/kg. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### Determining Abundance of the Proteins of the Invention by Imaging Technology

An advantage of determining abundance of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 by imaging technology may be that such a method is non-invasive (save that reagents may need to be administered) and there is no need to extract a sample from the subject.

Suitable imaging technologies include positron emission tomography (PET) and single photon emission computed tomography (SPECT). Visualisation of an OGTA using such techniques requires incorporation or binding of a suitable label e.g. a radiotracer such as ¹⁸F, ¹¹C or ¹²³I (see e.g. NeuroRx—The Journal of the American Society for Experimental NeuroTherapeutics (2005) 2(2), 348-360 and idem pages 361-371 for further details of the techniques). Radiotracers or other labels may be incorporated into OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 or OGTA271 by administration to the subject (e.g. by injection) of a suitably labelled specific ligand. Alternatively they may be incorporated into a binding affinity reagent (antibody, Affibody etc.) specific for an OGTA which may be administered to the subject (e.g. by injection). For discussion of use of Affibodies for imaging see e.g. Orlova A, Magnusson M, Eriksson T L, Nilsson M, Larsson B, Hoiden-Guthenberg I, Widstrom C, Carlsson J, Tolmachev V, Stahl S, Nilsson F Y, Tumour imaging using a picomolar affinity HER2 binding affibody molecule, Cancer Res. 2006 Apr. 15; 66(8):4339-48).

Diagnosis and Treatment of B-cell Non-Hodgkin's Lymphoma, Breast Cancer, Cervical Cancer, Colorectal Cancer, Gastric Cancer, Glioblastoma, Hepatocellular Carcinoma, Lung Cancer, Lymphoid Leukaemia (Particularly Acute T-cell Leukaemia and Chronic Lymphocytic Leukaemia), Melanoma, Neuroblastoma, Osteosarcoma, Ovarian Cancer, Pancreatic Cancer, Prostate Cancer, Renal Cell Cancer and Retinoblastoma using Immunohistochemistry

Immunohistochemistry is an excellent detection technique and may therefore be very useful in the diagnosis and treatment of a relevant cancer. Immunohistochemistry may be used to detect, diagnose, or monitor a relevant cancer through the localization of OGTA antigens in tissue sections by the use of labeled antibodies (or other affinity reagents), derivatives and analogs thereof, which specifically bind to the proteins of the invention, as specific reagents through antigen-antibody interactions that are visualized by a marker such as fluorescent dye, enzyme, radioactive element or colloidal gold.

The advancement of monoclonal antibody technology has been of great significance in assuring the place of immunohistochemistry in the modern accurate microscopic diagnosis of human neoplasms. The identification of disseminated neoplastically transformed cells by immunohistochemistry allows for a clearer picture of cancer invasion and metastasis, as well as the evolution of the tumour cell associated immunophenotype towards increased malignancy. Future antineoplastic therapeutical approaches may include a variety of individualized immunotherapies, specific for the particular immunophenotypical pattern associated with each individual patient's neoplastic disease. For further discussion see e.g. Bodey B, The significance of immunohistochemistry in the diagnosis and therapy of neoplasms, Expert Opin Biol Ther. 2002 April; 2(4):371-93.

Preferred features of each aspect of the invention are as for each of the other aspects mutatis mutandis. The prior art documents mentioned herein are incorporated to the fullest extent permitted by law.

### Example 1

### Identification of Membrane Proteins Expressed in B-Cell Non-Hodgkin's Lymphoma Breast Cancer Cervical Cancer Colorectal Cancer, Gastric Cancer Glioblastoma Hepatocellular Carcinoma Lung Cancer, Lymphoid Leukaemia (Particularly Acute T-Cell Leukaemia and Chronic Lymphocytic Leukaemia), Melanoma, Neuroblastoma, Osteosarcoma, Ovarian Cancer, Pancreatic Cancer, Prostate Cancer, Renal Cell Cancer and Retinoblastoma Blood and Tissue Samples

Using the following Reference Protocol, membrane proteins extracted from B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma tissue samples were separated by 1D gel and analysed.

### 1.1 Materials and Methods

### 1.1.1—Plasma Membrane Fractionation

The cells recovered from a B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma were lysed and submitted to centrifugation at 1000G. The supernatant was taken, and it was subsequently centrifuged at 3000G. Once again, the supernatant was taken, and it was then centrifuged at 100 000G.

The resulting pellet was recovered and put on 15-60% sucrose gradient.

A Western blot was used to identify sub cellular markers, and the Plasma Membrane fractions were pooled.

The pooled solution was either run directly on 1D gels (see section 1.1.4 below), or further fractionated into heparin binding and nucleotide binding fractions as described below.

### 1.1.2—Plasma Membrane Heparin-binding Fraction

The pooled solution from 1.1.1 above was applied to a Heparin column, eluted from column and run on 1D gels (see section 1.1.4 below).

### 1.1.3—Plasma Nucleotide-binding Fraction

The pooled solution from 1.1.1 above was applied to a Cibacrom Blue 3GA column, eluted from column and run on 1D gels (see section 1.1.4 below).

### 1.1.4—1D Gel Technology

Protein or membrane pellets were solubilised in 1D sample buffer (1-2 μg/μl). The sample buffer and protein mixture was then heated to 95° C. for 3 min.

A 9-16% acrylamide gradient gel was cast with a stacking gel and a stacking comb according to the procedure described in Ausubel F. M. et al., eds., 1989, Current Protocols in Molecular Biology, Vol. II, Green Publishing Associates, Inc., and John Wiley & Sons, Inc., New York, section 10.2, incorporated herein by reference in its entirety.

30-50 micrograms of the protein mixtures obtained from detergent and the molecular weight standards (66, 45, 31, 21, 14 kDa) were added to the stacking gel wells using a 10 microlitre pipette tip and the samples run at 40 mA for 5 hours.

The plates were then prised open, the gel placed in a tray of fixer (10% acetic acid, 40% ethanol, 50% water) and shaken overnight. Following this, the gel was primed by 30 minutes shaking in a primer solution (7.5% acetic acid (75 ml), 0.05% SDS (5 ml of 10%)). The gel was then incubated with a fluorescent dye (7.5% acetic acid, 0.06% OGS in-house dye (600 μl)) with shaking for 3 hrs. Sypro Red (Molecular Probes, Inc., Eugene, Oreg.) is a suitable dye for this purpose. A preferred fluorescent dye is disclosed in U.S. application Ser. No. 09/412,168, filed on Oct. 5, 1999, which is incorporated herein by reference in its entirety.

A computer-readable output was produced by imaging the fluorescently stained gels with an Apollo 3 scanner (Oxford Glycosciences, Oxford, UK). This scanner is developed from the scanner described in WO 96/36882 and in the Ph.D. thesis of David A. Basiji, entitled “Development of a High-throughput Fluorescence Scanner Employing Internal Reflection Optics and Phase-sensitive Detection (Total Internal Reflection, Electrophoresis)”, University of Washington (1997), Volume 58/12-B of Dissertation Abstracts International, page 6686, the contents of each of which are incorporated herein by reference. The latest embodiment of this instrument includes the following improvements: The gel is transported through the scanner on a precision lead-screw drive system. This is preferable to laying the glass plate on the belt-driven system that is defined in the Basiji thesis as it provides a reproducible means of accurately transporting the gel past the imaging optics.

The gel is secured into the scanner against three alignment stops that rigidly hold the glass plate in a known position. By doing this in conjunction with the above precision transport system and the fact that the gel is bound to the glass plate, the absolute position of the gel can be predicted and recorded. This ensures that accurate co-ordinates of each feature on the gel can be communicated to the cutting robot for excision. This cutting robot has an identical mounting arrangement for the glass plate to preserve the positional accuracy.

The carrier that holds the gel in place has integral fluorescent markers (Designated M1, M2, M3) that are used to correct the image geometry and are a quality control feature to confirm that the scanning has been performed correctly.

The optical components of the system have been inverted. The laser, mirror, waveguide and other optical components are now above the glass plate being scanned. The embodiment of the Basiji thesis has these underneath. The glass plate is therefore mounted onto the scanner gel side down, so that the optical path remains through the glass plate. By doing this, any particles of gel that may break away from the glass plate will fall onto the base of the instrument rather than into the optics.

In scanning the gels, they were removed from the stain, rinsed with water and allowed to air dry briefly and imaged on the Apollo 3. After imaging, the gels were sealed in polyethylene bags containing a small volume of staining solution, and then stored at 4° C.

Apparent molecular weights were calculated by interpolation from a set of known molecular weight markers run alongside the samples.

### 1.1.5—Recovery and Analysis of Selected Proteins

Proteins were robotically excised from the gels by the process described in U.S. Pat. No. 6,064,754, Sections 5.4 and 5.6, 5.7, 5.8 (incorporated herein by reference), as is applicable to 1D-electrophoresis, with modification to the robotic cutter as follows: the cutter begins at the top of the lane, and cuts a gel disc 1.7 mm in diameter from the left edge of the lane. The cutter then moves 2 mm to the right, and 0.7 mm down and cuts a further disc. This is then repeated. The cutter then moves back to a position directly underneath the first gel cut, but offset by 2.2 mm downwards, and the pattern of three diagonal cuts are repeated. This is continued for the whole length of the gel.

NOTE: If the lane is observed to broaden significantly then a correction can be made also sideways i.e. instead of returning to a position directly underneath a previous gel cut, the cut can be offset slightly to the left (on the left of the lane) and/or the right (on the right of the lane). The proteins contained within the gel fragments were processed to generate tryptic peptides; partial amino acid sequences of these peptides were determined by mass spectroscopy as described in WO98/53323 and application Ser. No. 09/094,996, filed Jun. 15, 1998.

Proteins were processed to generate tryptic digest peptides. Tryptic peptides were analyzed by mass spectrometry using a PerSeptive Biosystems Voyager-DETM STR Matrix-Assisted Laser Desorption Ionization Time-of-Flight (MALDI-TOF) mass spectrometer, and selected tryptic peptides were analyzed by tandem mass spectrometry (MS/MS) using a Micromass Quadrupole Time-of-Flight (Q-TOF) mass spectrometer (Micromass, Altrincham, U.K.) equipped with a Nanoflow™ electrospray Z-spray source. For partial amino acid sequencing and identification of OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 and OGTA271, uninterpreted tandem mass spectra of tryptic peptides were searched using the SEQUEST search program (Eng et al., 1994, J. Am. Soc. Mass Spectrom. 5:976-989), version v.C.1. Criteria for database identification included: the cleavage specificity of trypsin; the detection of a suite of a, b and y ions in peptides returned from the database, and a mass increment for all Cys residues to account for carbamidomethylation. The database searched was a database constructed of protein entries in the non-redundant database held by the National Centre for Biotechnology Information (NCBI) which is accessible at worldwide web ncbi.nlm.nih.gov. Following identification of proteins through spectral-spectral correlation using the SEQUEST program, masses detected in MALDI-TOF mass spectra were assigned to tryptic digest peptides within the proteins identified. In cases where no amino acid sequences could be identified through searching with uninterpreted MS/MS spectra of tryptic digest peptides using the SEQUEST program, tandem mass spectra of the peptides were interpreted manually, using methods known in the art. (In the case of interpretation of low-energy fragmentation mass spectra of peptide ions see Gaskell et al., 1992, Rapid Commun. Mass Spectrom. 6:658-662).

1.1.6—Discrimination of B-Cell Non-Hodgkin's Lymphoma, Breast Cancer, Cervical Cancer, Colorectal Cancer, Gastric Cancer, Glioblastoma, Hepatocellular Carcinoma, Lung Cancer, Lymphoid Leukaemia (Particularly Acute T-Cell Leukaemia and Chronic Lymphocytic Leukaemia), Melanoma, Neuroblastoma, Osteosarcoma, Ovarian Cancer, Pancreatic Cancer, Prostate Cancer, Renal Cell Cancer and Retinoblastoma Associated Proteins

The process to identify OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 and OGTA271 uses the peptide sequences obtained experimentally by mass spectrometry described above of naturally occurring human proteins to identify and organise coding exons in the published human genome sequence.

Recent dramatic advances in defining the chemical sequence of the human genome have led to the near completion of this immense task (Venter, J. C. et al. (2001). The sequence of the human genome. Science 16: 1304-51; International Human Genome Sequencing Consortium. (2001). Initial sequencing and analysis of the human genome Nature 409: 860-921). There is little doubt that this sequence information will have a substantial impact on our understanding of many biological processes, including molecular evolution, comparative genomics, pathogenic mechanisms and molecular medicine. For the full medical value inherent in the sequence of the human genome to be realised, the genome needs to be ‘organised’ and annotated. By this, is meant at least the following three things: (i) The assembly of the sequences of the individual portions of the genome into a coherent, continuous sequence for each chromosome. (ii) The unambiguous identification of those regions of each chromosome that contain genes. (iii) Determination of the fine structure of the genes and the properties of its mRNA and protein products. While the definition of a ‘gene’ is an increasingly complex issue (H Pearson: What is a gene? Nature (2006) 24: 399-401), what is of immediate interest for drug discovery and development is a catalogue of those genes that encode functional, expressed proteins. A subset of these genes will be involved in the molecular basis of most if not all pathologies. Therefore an important and immediate goal for the pharmaceutical industry is to identify all such genes in the human genome and describe their fine structure.

### Processing and Integration of Peptide Masses Peptide Signatures ESTs and Public Domain Genomic Sequence Data to Form OGAP® Database

Discrete genetic units (exons, transcripts and genes) were identified using the following sequential steps:

1. A ‘virtual transcriptome’ is generated, containing the tryptic peptides which map to the human genome by combining the gene identifications available from Ensembl and various gene prediction programs. This also incorporates SNP data (from dbSNP) and all alternate splicing of gene identifications. Known contaminants were also added to the virtual transcriptome.
2. All tandem spectra in the OGeS Mass Spectrometry Database are interpreted in order to produce a peptide that can be mapped to one in the virtual transcriptome. A set of automated spectral interpretation algorithms were used to produce the peptide identifications.
3. The set of all mass-matched peptides in the OGeS Mass Spectrometry Database is generated by searching all peptides from transcripts hit by the tandem peptides using a tolerance based on the mass accuracy of the mass spectrometer, typically 20 ppm.
4. All tandem and mass-matched peptides are combined in the form of “protein clusters”. This is done using a recursive process which groups sequences into clusters based on common peptide hits. Biological sequences are considered to belong to the same cluster if they share one or more tandem or mass-matched peptide.
5. After initial filtering to screen out incorrectly identified peptides, the resulting clusters are then mapped on the human genome.
6. The protein clusters are then aggregated into regions that define preliminary gene boundaries using their proximity and the co-observation of peptides within protein clusters. Proximity is defined as the peptide being within 80,000 nucleotides on the same strand of the same chromosome. Various elimination rules, based on cluster observation scoring and multiple mapping to the genome are used to refine the output. The resulting ‘confirmed genes’ are those which best account for the peptides and masses observed by mass spectrometry in each cluster. Nominal co-ordinates for the gene are also an output of this stage.
7. The best set of transcripts for each confirmed gene are created from the protein clusters, peptides, ESTs, candidate exons and molecular weight of the original protein spot.
8. Each identified transcript was linked to the sample providing the observed peptides.
9. Use of an application for viewing and mining the data. The result of steps 1-8 was a database containing genes, each of which consisted of a number of exons and one or more transcripts. An application was written to display and search this integrated genome/proteome data. Any features (OMIM disease locus, InterPro etc.) that had been mapped to the same Golden Path co-ordinate system by Ensembl could be cross-referenced to these genes by coincidence of location and fine structure.

The process was used to generate approximately 1 million peptide sequences to identify protein-coding genes and their exons resulted in the identification of protein sequences for 18083 genes across 67 different tissues and 57 diseases including 2,025 genes in acute T-cell leukaemia, 501 genes in B-cell non-Hodgkin's lymphoma, 4,713 genes in breast cancer, 1,371 genes in cervical cancer, 2,424 genes in chronic lymphocytic leukaemia, 949 genes in colorectal cancer, 524 genes in gastric cancer, 1,544 genes in glioblastoma, 1,782 genes in hepatocellular carcinoma, 978 genes in lung cancer, 373 genes in lymphoid leukaemia (unspecified), 1,764 genes in melanoma, 1,391 genes in neuroblastoma, 1,324 genes in osteosarcoma, 1,033 genes in ovarian cancer, 2,961 genes in pancreatic cancer, 3,307 genes in prostate cancer, 1005 genes in renal cell cancer and 1,783 genes in retinoblastoma, illustrated here by OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 and OGTA271 isolated and identified from B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma samples. Following comparison of the experimentally determined sequences with sequences in the OGAP® database, OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 and OGTA271 showed a high degree of specificity to B-cell non-Hodgkin's lymphoma, breast cancer, cervical cancer, colorectal cancer, gastric cancer, glioblastoma, hepatocellular carcinoma, lung cancer, lymphoid leukaemia (particularly acute T-cell leukaemia and chronic lymphocytic leukaemia), melanoma, neuroblastoma, osteosarcoma, ovarian cancer, pancreatic cancer, prostate cancer, renal cell cancer and retinoblastoma indicative of the prognostic and diagnostic nature.

### 1.2 Results

These experiments identified OGTA002, OGTA009, OGTA016, OGTA028, OGTA037, OGTA041, OGTA053, OGTA054, OGTA066, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 and OGTA271, as further described herein.

The full-length OGTA002 was detected in the plasma membrane of breast cancer, colorectal cancer, hepatocellular carcinoma, melanoma, ovarian cancer and pancreatic cancer samples. The full-length OGTA009 was detected in the plasma membrane of breast cancer, colorectal cancer, pancreatic cancer, prostate cancer and renal cell cancer samples. The full-length OGTA016 was detected in the plasma membrane of colorectal cancer samples. The full-length OGTA028 was detected in the plasma membrane of ovarian cancer samples. The full-length OGTA037 was detected in the plasma membrane of gastric cancer samples. The full-length OGTA041 was detected in the plasma membrane of hepatocellular carcinoma, lung cancer and pancreatic cancer samples. The full-length OGTA053 was detected in the plasma membrane of pancreatic cancer samples. The full-length OGTA054 was detected in the plasma membrane of pancreatic cancer samples. The full-length OGTA066 was detected in the plasma membrane of colorectal cancer and pancreatic cancer samples. The full-length OGTA072 was detected in the plasma membrane of colorectal cancer samples. The full-length OGTA074 was detected in the plasma membrane of colorectal cancer samples. The full-length OGTA076 was detected in the plasma membrane of chronic lymphocytic leukaemia, colorectal cancer and pancreatic cancer samples. The full-length OGTA085 was detected in the plasma membrane of lung cancer, melanoma and retinoblastoma samples. The full-length OGTA087 was detected in the plasma membrane of B-cell non-Hodgkin's lymphoma, breast cancer, colorectal cancer, gastric cancer, lung cancer, lymphoid leukaemia and ovarian cancer samples. The full-length OGTA088 was detected in the plasma membrane of breast cancer, gastric cancer, glioblastoma, hepatocellular carcinoma, lung cancer, lymphoid leukaemia, pancreatic cancer, renal cell cancer and retinoblastoma samples. The full-length OGTA089 was detected in the plasma membrane of breast cancer, lung cancer and ovarian cancer samples. The full-length OGTA091 was detected in the plasma membrane of breast cancer, cervical cancer, chronic lymphocytic leukaemia, gastric cancer, hepatocellular carcinoma, lung cancer, melanoma, osteosarcoma, ovarian cancer, pancreatic cancer, prostate cancer and renal cell cancer samples. The full-length OGTA098 was detected in the plasma membrane of breast cancer, cervical cancer, hepatocellular carcinoma, lung cancer, osteosarcoma, pancreatic cancer, prostate cancer and renal cell cancer samples. The full-length OGTA101 was detected in the plasma membrane of breast cancer, cervical cancer, colorectal cancer, hepatocellular carcinoma, lung cancer, osteosarcoma and pancreatic cancer samples. The full-length OGTA104 was detected in the plasma membrane of breast cancer, hepatocellular carcinoma, ovarian cancer and pancreatic cancer samples. The full-length OGTA106 was detected in the plasma membrane of cervical cancer, melanoma and pancreatic cancer samples. The full-length OGTA112 was detected in the plasma membrane of breast cancer, cervical cancer, chronic lymphocytic leukaemia, hepatocellular carcinoma, pancreatic cancer and renal cell cancer samples. The full-length OGTA113 was detected in the plasma membrane of pancreatic cancer samples. The full-length OGTA119 was detected in the plasma membrane of B-cell non-Hodgkin's lymphoma, breast cancer, colorectal cancer, gastric cancer, hepatocellular carcinoma, lung cancer, lymphoid leukaemia, neuroblastoma, osteosarcoma, pancreatic cancer, prostate cancer and renal cell cancer samples. The full-length OGTA124 was detected in the plasma membrane of hepatocellular carcinoma, lung cancer, ovarian cancer, pancreatic cancer and renal cell cancer samples. The full-length OGTA126 was detected in the plasma membrane of breast cancer, colorectal cancer, hepatocellular carcinoma, melanoma, pancreatic cancer, prostate cancer and renal cell cancer samples. The full-length OGTA156 was detected in the plasma membrane of acute T-cell leukaemia, B-cell non-Hodgkin's lymphoma, chronic lymphocytic leukaemia and hepatocellular carcinoma samples. The full-length OGTA159 was detected in the plasma membrane of breast cancer, chronic lymphocytic leukaemia, colorectal cancer, hepatocellular carcinoma, melanoma and pancreatic cancer samples. The full-length OGTA168 was detected in the plasma membrane of glioblastoma and melanoma samples. The full-length OGTA169 was detected in the plasma membrane of breast cancer and chronic lymphocytic leukaemia samples. The full-length OGTA174 was detected in the plasma membrane of acute T-cell leukaemia and chronic lymphocytic leukaemia samples. The full-length OGTA176 was detected in the plasma membrane of B-cell non-Hodgkin's lymphoma and chronic lymphocytic leukaemia samples. The full-length OGTA177 was detected in the plasma membrane of chronic lymphocytic leukaemia samples. The full-length OGTA197 was detected in the plasma membrane of colorectal cancer, hepatocellular carcinoma, lung cancer, melanoma, osteosarcoma, ovarian cancer, pancreatic cancer, prostate cancer and renal cell cancer samples. The full-length OGTA202 was detected in the plasma membrane of colorectal cancer samples. The full-length OGTA203 was detected in the plasma membrane of ovarian cancer and renal cell cancer samples. The full-length OGTA206 was detected in the plasma membrane of breast cancer, pancreatic cancer and prostate cancer samples. The full-length OGTA213 was detected in the plasma membrane of lung cancer samples. The full-length OGTA214 was detected in the plasma membrane of ovarian cancer samples. The full-length OGTA216 was detected in the plasma membrane of breast cancer, colorectal cancer and pancreatic cancer samples. The full-length OGTA222 was detected in the plasma membrane of B-cell non-Hodgkin's lymphoma and colorectal cancer samples. The full-length OGTA236 was detected in the plasma membrane of pancreatic cancer samples. The full-length OGTA237 was detected in the plasma membrane of B-cell non-Hodgkin's lymphoma, lymphoid leukaemia and prostate cancer samples. The full-length OGTA247 was detected in the plasma membrane of hepatocellular carcinoma and melanoma samples. The full-length OGTA248 was detected in the plasma membrane of renal cell cancer samples. The full-length OGTA249 was detected in the plasma membrane of ovarian cancer samples. The full-length OGTA257 was detected in the plasma membrane of pancreatic cancer samples. The full-length OGTA271 was detected in the plasma membrane of breast cancer, cervical cancer, colorectal cancer, hepatocellular carcinoma, lung cancer, osteosarcoma, pancreatic cancer and renal cell cancer samples.

### Example 2

### Identification of Membrane Proteins Expressed in Colorectal Cancer, Kidney Cancer, Liver Cancer, Lung Cancer or Ovarian Cancer Blood And Tissue Samples Using Isotope Tagging for Absolute and Relative Quantitation (iTRAQ)

Using the following Reference Protocol, membrane proteins extracted from colorectal cancer, kidney cancer, liver cancer, lung cancer and ovarian cancer tissue and normal adjacent colorectal and lung tissue samples were digested, labelled with Isotope Tagging for Absolute & Relative Quantitation reagents (iTRAQ; Applied Biosystems, Foster City, Calif., USA) and resulting peptides sequenced by tandem mass spectrometry.

### 2.1 Materials and Methods

### 2.1.1—Plasma Membrane Fractionation

The cells recovered from a colorectal cancer, kidney cancer, liver cancer, lung cancer or ovarian cancer or normal adjacent colorectal, kidney, liver, lung or ovarian tissue were lysed and submitted to centrifugation at 1000G. The supernatant was taken, and it was subsequently centrifuged at 3000G. Once again, the supernatant was taken, and it was then centrifuged at 100 000G.

The resulting pellet was recovered and put on 15-60% sucrose gradient.

A Western blot was used to identify sub cellular markers, and the Plasma Membrane fractions were pooled.

The pooled solution was then analysed directly by iTRAQ (see section 2.1.2 below).

2.1.2—iTRAQ Methodology

Membrane protein pellets from colorectal cancer, kidney cancer, liver cancer, lung cancer or ovarian cancer and normal adjacent colorectal, kidney, liver, lung or ovarian tissue were solubilised in sample buffer (2-4 μg/Win 0.5% SDS) by the addition of buffer and then heating to 95° C. for 3 min.

To a volume of each protein solution equating to 50 μg, 150 μl of 0.5M triethylammonium bicarbonate (TEAB) solution was added. To each sample, 3 μl of 50 mM tris-(2-carboxyethyl)phosphine was added and the mixture was incubated at 60° C. for 1 hour. 1 μl of cysteine blocking reagent, 200 mM methyl methanethiosulphonate (MMTS) in isopropanol, was then added. After incubation at room temperature for 10 minutes, 15 μl of 1 μg/μl trypsin was added to each sample followed by incubation at 37° C. overnight.

The digested samples were dried under a vacuum and re-constituted with 30 μl of 0.5M TEAB solution. 70 μl ethanol was added to each of the four iTRAQ reagents (114/115/116/117) and one reagent added to each of the four samples analysed (two colorectal cancer, kidney cancer, liver cancer, lung cancer or ovarian cancer samples and two corresponding normal adjacent tissue samples) and left at room temperature for 1 hour. The specific reagent added to each sample was recorded. The four labeled samples were combined & vortexed.

The combined sample was reduced to dryness under a vacuum and de-salted by loading onto a C18 spin column, washing with aqueous solvent and then eluting with 70% acetonitrile. The sample fraction was again reduced to dryness and then re-dissolved in 40 μl of solvent A (97.9 water, 2% acetonitrile, 0.1% formic acid) prior to ion exchange fractionation.

### 2.1.3. Fractionation and Analysis of Labeled Peptides

The sample was fractionated by strong cation exchange chromatography using an Agilent 1200 chromatograph (Agilent, Santa Clara, Calif., USA). Samples were eluted off an Agilent Zorbax Bio-SCXII column (3.5 μm; 50×0.8 mm) using a 200 min gradient of 0-100 mM sodium acetate over 20 minutes and then to 1M over 10 minutes. 1 minute fractions were collected over the 30 minute run.

Each fraction was analysed by liquid chromatography/mass spectrometry using an Agilent 1200 chromatograph fitted with a Zorbax 3005B-C18 (150 mm×75 μm) and an Agilent 6510 quadrupole—time-of-flight instrument (Agilent, Santa Clara, Calif., USA). Peptides were eluted with a 300n1/min gradient increasing from 15% to 45% acetonitrile in 60 minutes. Data was acquired in auto MS/MS mode such that up to 3 precursor ions above the intensity threshold were selected and product ion spectra accumulated to facilitate the sequencing of the labeled peptides. Raw was processed to create peak lists using Spectrum Mill software (Agilent, Santa Clara, Calif., USA).

### 2.1.4-Amino Acid Sequence Analysis of Labeled Peptides

For partial amino acid sequencing and identification of OGTA002, OGTA009, OGTA016, OGTA028, OGTA037, OGTA041, OGTA053, OGTA054, OGTA066, OGTA074, OGTA076, OGTA085, OGTA088, OGTA089, OGTA091, OGTA098, OGTA101, OGTA104, OGTA106, OGTA112, OGTA113, OGTA119, OGTA124, OGTA126, OGTA156, OGTA159, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 and OGTA271, uninterpreted tandem mass spectra of tryptic peptides were searched using the SEQUEST search program (Eng et al., 1994, J. Am. Soc. Mass Spectrom. 5:976-989). Criteria for database identification included: the cleavage specificity of trypsin; the detection of a suite of a, b and y ions in peptides returned from the database, and a mass increment for all cysteine residues to account for modification with methyl methanethiosulphonate and the addition of iTRAQ labels to free amines (N-terminus & lysine). The data was searched through IPI Human v3.23 (worldwide web ebi.ac.uk/IPI/IPIhuman.html).

### 2.1.5—Discrimination of Colorectal Cancer, Kidney Cancer, Liver Cancer, Lung Cancer and Ovarian Cancer Associated Proteins

The process described in Example 1 section 1.1.6 was employed to discriminate the colorectal cancer, kidney cancer, liver cancer, lung cancer and ovarian cancer associated proteins in the experimental samples.

### 2.2 Results

These experiments identified OGTA002, OGTA009, OGTA016, OGTA028, OGTA037, OGTA041, OGTA053, OGTA054, OGTA066, OGTA074, OGTA076, OGTA085, OGTA088, OGTA089, OGTA091, OGTA098, OGTA101, OGTA104, OGTA106, OGTA112, OGTA113, OGTA119, OGTA124, OGTA126, OGTA156, OGTA159, OGTA169, OGTA174, OGTA176, OGTA177, OGTA197, OGTA202, OGTA203, OGTA206, OGTA213, OGTA214, OGTA216, OGTA222, OGTA236, OGTA237, OGTA247, OGTA248, OGTA249, OGTA257 and OGTA271, as further described herein.

The full-length OGTA002 was detected in the plasma membrane of colorectal cancer, hepatocellular carcinoma, lung cancer and renal cell cancer samples. The full-length OGTA009 was detected in the plasma membrane of colorectal cancer, lung cancer and ovarian cancer samples. The full-length OGTA016 was detected in the plasma membrane of colorectal cancer and lung cancer samples. The full-length OGTA028 was detected in the plasma membrane of colorectal cancer, hepatocellular carcinoma, lung cancer, ovarian cancer and renal cell cancer samples. The full-length OGTA037 was detected in the plasma membrane of colorectal cancer, lung cancer and ovarian cancer samples. The full-length OGTA041 was detected in the plasma membrane of lung cancer samples. The full-length OGTA053 was detected in the plasma membrane of colorectal cancer, hepatocellular carcinoma, lung cancer and renal cell cancer samples. The full-length OGTA054 was detected in the plasma membrane of colorectal cancer and lung cancer samples. The full-length OGTA066 was detected in the plasma membrane of colorectal cancer, lung cancer and renal cell cancer samples. The full-length OGTA074 was detected in the plasma membrane of lung cancer samples. The full-length OGTA076 was detected in the plasma membrane of colorectal cancer, hepatocellular carcinoma, lung cancer, ovarian cancer and renal cell cancer samples. The full-length OGTA085 was detected in the plasma membrane of colorectal cancer and lung cancer samples. The full-length OGTA088 was detected in the plasma membrane of colorectal cancer and lung cancer samples. The full-length OGTA089 was detected in the plasma membrane of lung cancer, ovarian cancer and renal cell cancer samples. The full-length OGTA091 was detected in the plasma membrane of breast colorectal cancer, hepatocellular carcinoma, lung cancer, ovarian cancer and renal cell cancer samples. The full-length OGTA098 was detected in the plasma membrane of colorectal cancer, lung cancer, ovarian cancer and renal cell cancer samples. The full-length OGTA101 was detected in the plasma membrane of colorectal cancer, hepatocellular carcinoma, lung cancer, ovarian cancer and renal cell cancer samples. The full-length OGTA104 was detected in the plasma membrane of colorectal cancer, hepatocellular carcinoma, lung cancer, ovarian cancer and renal cell cancer samples. The full-length OGTA106 was detected in the plasma membrane of hepatocellular carcinoma samples. The full-length OGTA112 was detected in the plasma membrane of renal cell cancer samples. The full-length OGTA113 was detected in the plasma membrane of colorectal cancer, lung cancer, ovarian cancer and renal cell cancer samples. The full-length OGTA119 was detected in the plasma membrane of colorectal cancer, lung cancer, and ovarian cancer samples. The full-length OGTA124 was detected in the plasma membrane of colorectal cancer, hepatocellular carcinoma, lung cancer, ovarian cancer and renal cell cancer samples. The full-length OGTA126 was detected in the plasma membrane of lung cancer samples. The full-length OGTA156 was detected in the plasma membrane of colorectal cancer, lung cancer, ovarian cancer and renal cell cancer samples. The full-length OGTA159 was detected in the plasma membrane of lung cancer and renal cell cancer samples. The full-length OGTA169 was detected in the plasma membrane of colorectal cancer, lung cancer and renal cell cancer samples. The full-length OGTA174 was detected in the plasma membrane of lung cancer samples. The full-length OGTA176 was detected in the plasma membrane of colorectal cancer, lung cancer and renal cell cancer samples. The full-length OGTA177 was detected in the plasma membrane of colorectal cancer, lung cancer, ovarian cancer and renal cell cancer samples. The full-length OGTA197 was detected in the plasma membrane of colorectal cancer, hepatocellular carcinoma, lung cancer and renal cell cancer samples. The full-length OGTA202 was detected in the plasma membrane of colorectal cancer and lung cancer samples. The full-length OGTA₂O₃ was detected in the plasma membrane of lung cancer and renal cell cancer samples. The full-length OGTA206 was detected in the plasma membrane of colorectal cancer and lung cancer samples. The full-length OGTA213 was detected in the plasma membrane of colorectal cancer and lung cancer samples. The full-length OGTA214 was detected in the plasma membrane of colorectal cancer samples. The full-length OGTA216 was detected in the plasma membrane of colorectal cancer, hepatocellular carcinoma, lung cancer, ovarian cancer and renal cell cancer samples. The full-length OGTA222 was detected in the plasma membrane of colorectal cancer, lung cancer and renal cell cancer samples. The full-length OGTA236 was detected in the plasma membrane of colorectal cancer samples. The full-length OGTA237 was detected in the plasma membrane of colorectal cancer, lung cancer and renal cell cancer samples. The full-length OGTA247 was detected in the plasma membrane of lung cancer samples. The full-length OGTA248 was detected in the plasma membrane of lung cancer samples. The full-length OGTA249 was detected in the plasma membrane of lung cancer and renal cell cancer samples. The full-length OGTA257 was detected in the plasma membrane of colorectal cancer, hepatocellular carcinoma, lung cancer and ovarian cancer samples. The full-length OGTA271 was detected in the plasma membrane of colorectal cancer, hepatocellular carcinoma, lung cancer and renal cell cancer samples.

### Example 3

### Evaluation of Colorectal Cancer Marker Proteins in Sandwich ELISA

Using the following Reference Protocol, colorectal cancer marker proteins, including OGTA066 were evaluated in a sandwich ELISA.

### 3.1 Materials and Methods

Antibodies for the sandwich ELISAs were developed at Biosite. Biotinylated antibody (primary antibody) was diluted into assay buffer (10 mM Tris, 150 mM NaCl, 1% BSA) to 2 ug/ml and added to 384 well neutravidin coated plate (Pierce Chemical Company, Rockford Ill.) and allowed to incubate at room temperature for 1 hour. Wells were then washed with wash buffer (20 mM Borate, 150 mM NaCl, 0.2% Tween 20). Samples and standards were added and allowed to incubate at room temperature for 1 hour. Wells again were washed. An antibody conjugated to fluorscein (secondary antibody) was diluted into assay buffer to 2 ug/ml and was then added to the plate and allowed to incubate at room temperature for 1 hour. Wells again were washed. Anti-fluorscein antibody conjugated to alkaline phosphatase, diluted 1/2338 into assay buffer, was added and allowed to incubate at room temperature for 1 hour. Final wash was then performed. Finally substrate (Promega Attophos Product#S1011, Promega Corporation, Madison, Wis.) was added and the plate was read immediately. All additions were 10 ul/well. The plate was washed 3 times between each addition and final wash was 9 times prior to the addition of substrate. Standards were prepared by spiking specific antigen into a normal serum patient pool. Reading was performed using a Tecan Spectrafluor plus (Tecan Inc, Mannedorf, Switzerland) in kinetic mode for 6 read cycles with excitation filter of 430 nm and an emission filter 570 nm emission. Slope of RFU/seconds was determined.

Final Box and ROC results were analyzed using Analyse-it General+Clinical Laboratory 1.73 (Analyse-it Software Ltd., Leeds England).

### 3.2 Results

These experiments identified proteins of particular interest including, but not limited to, OGTA066 (SEQ ID No: 9).

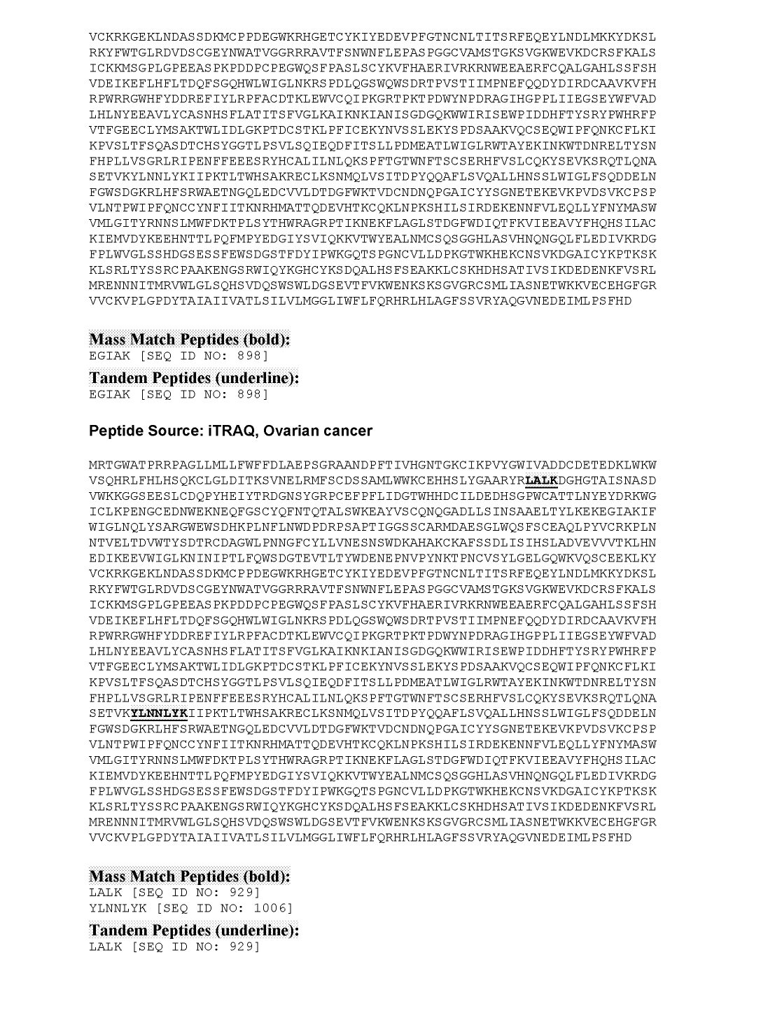

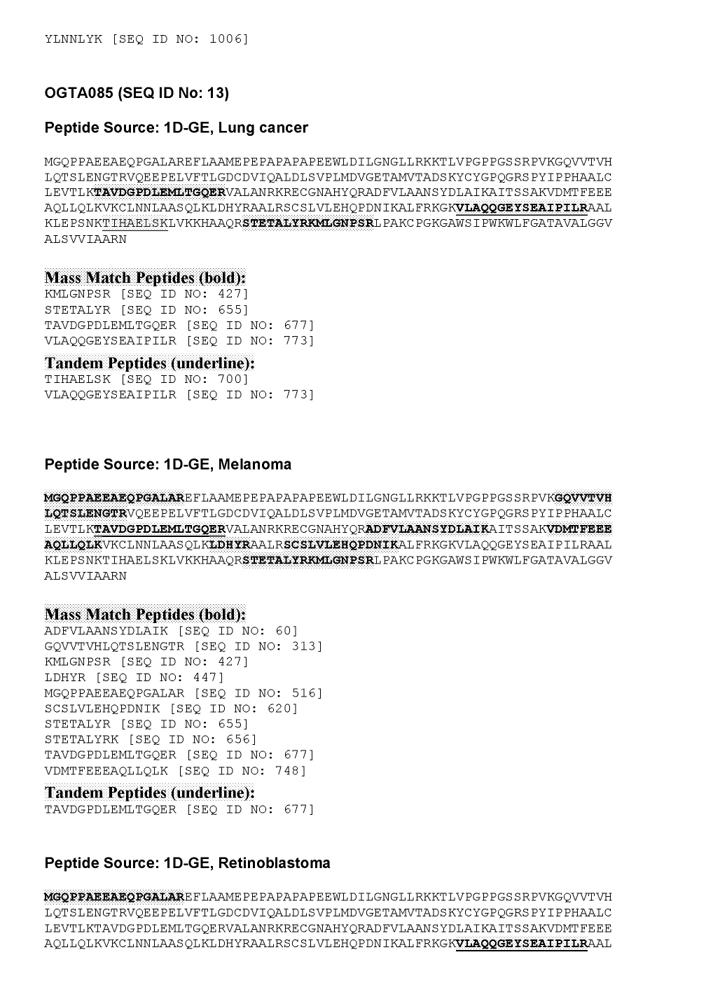

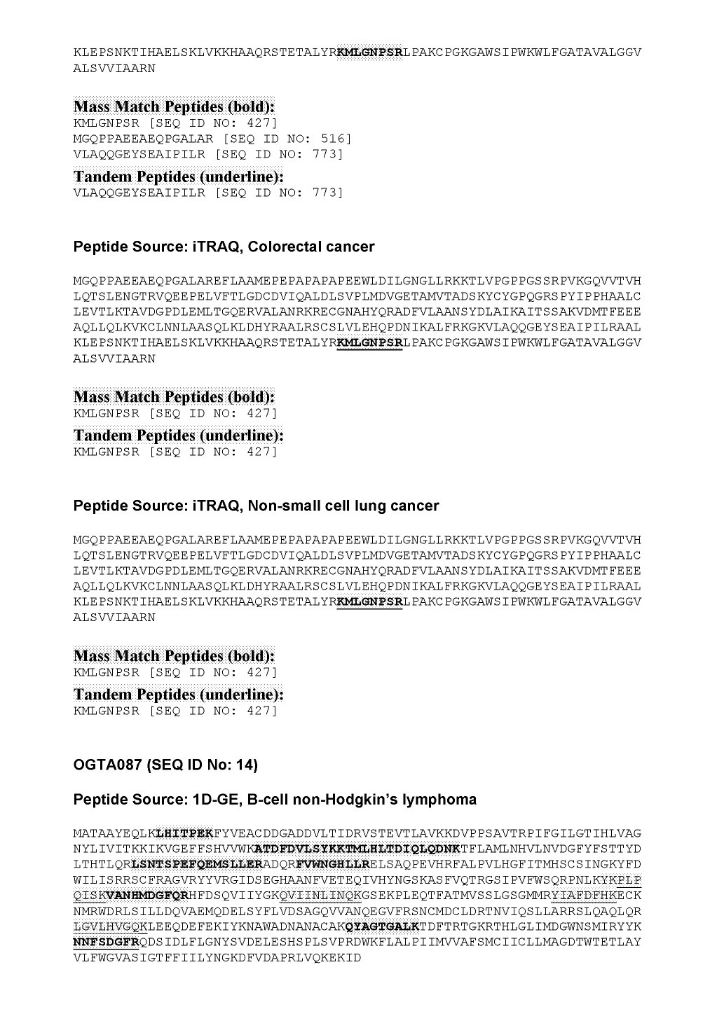

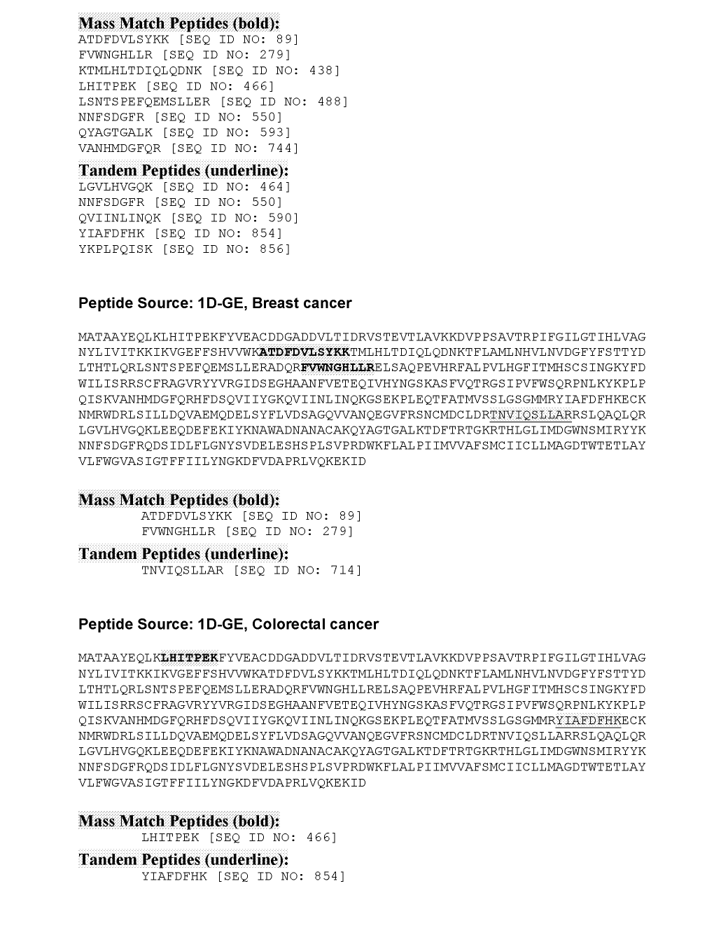

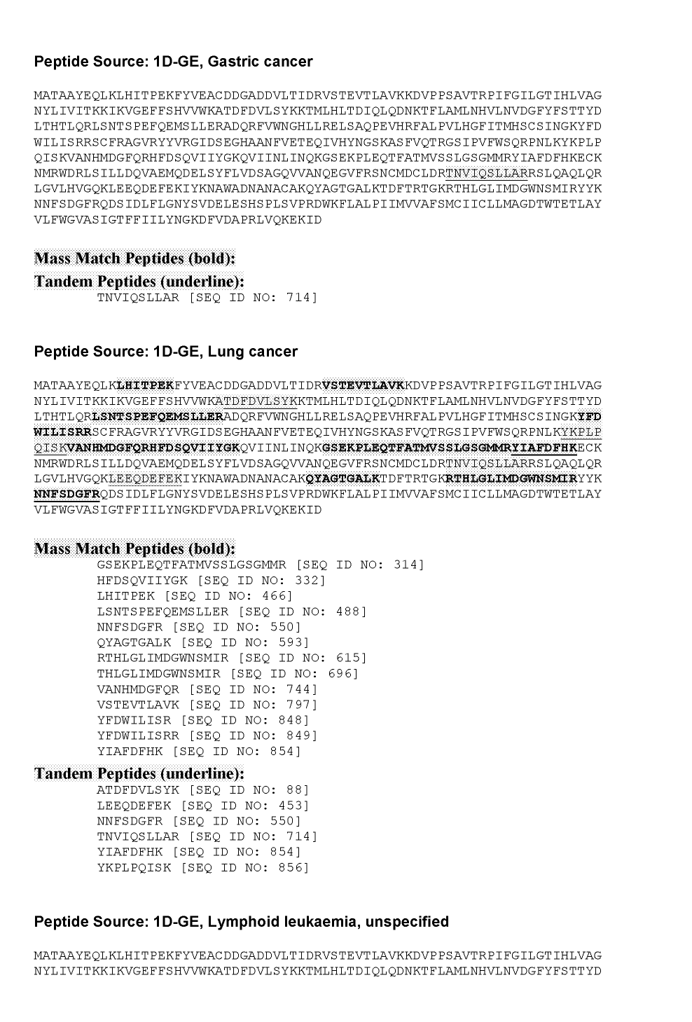

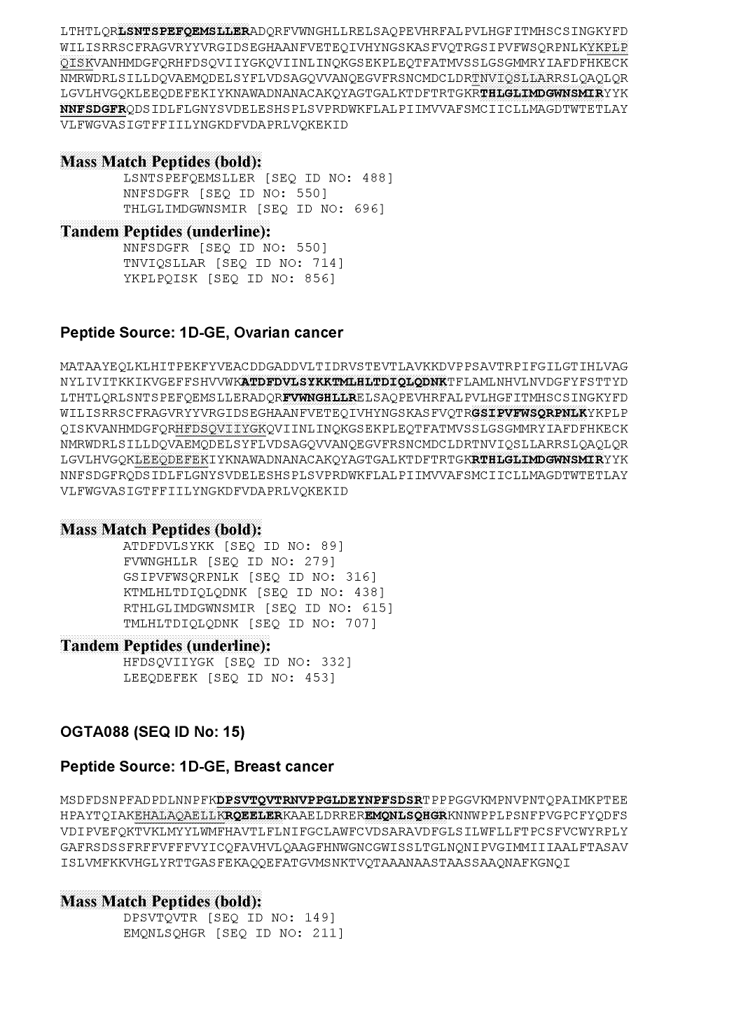

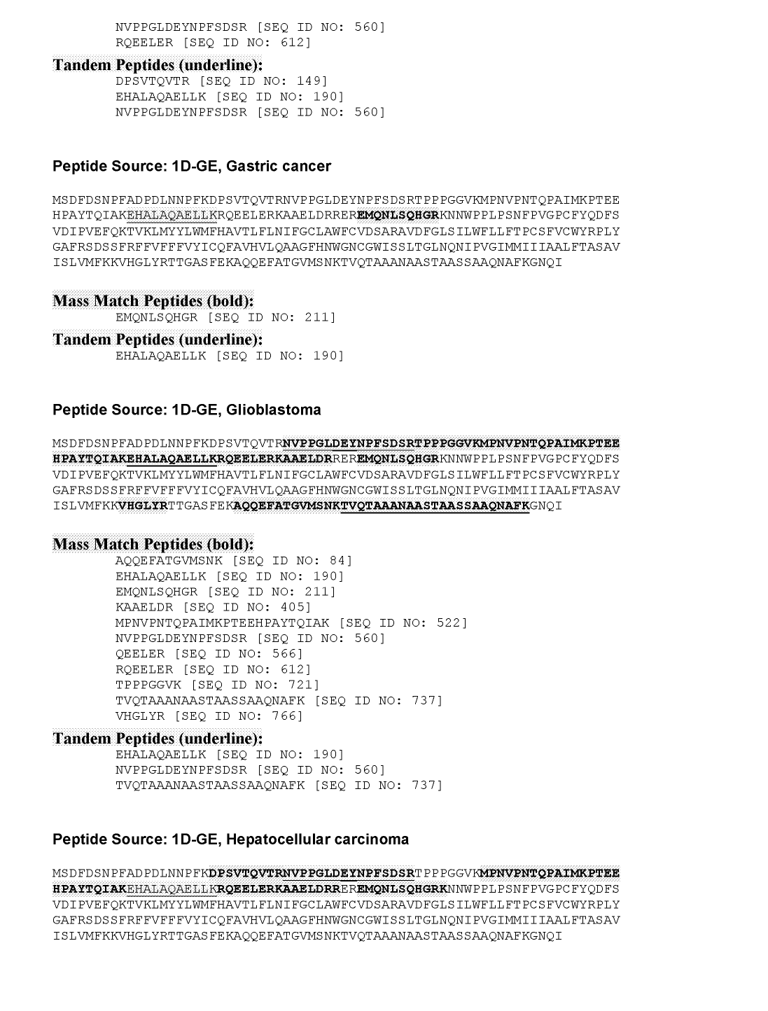

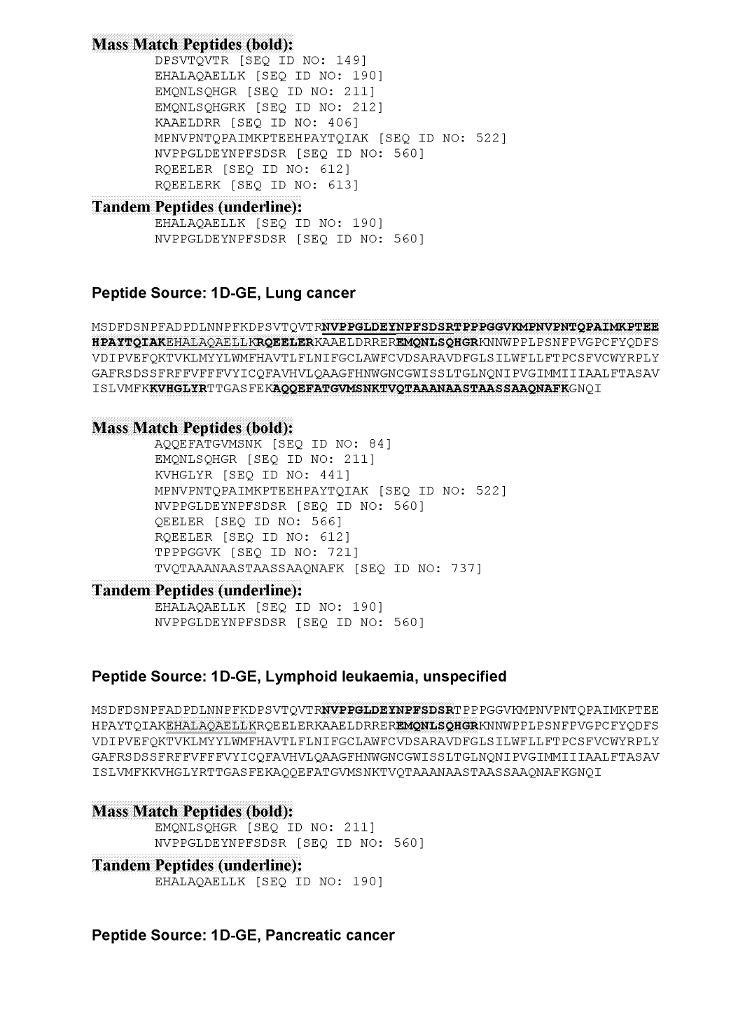

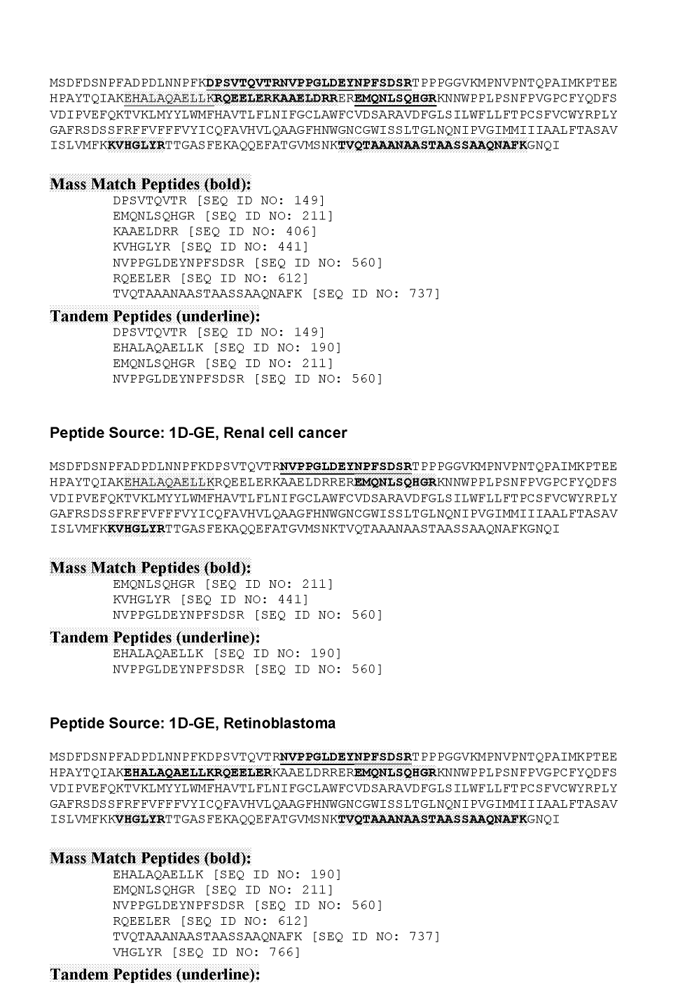

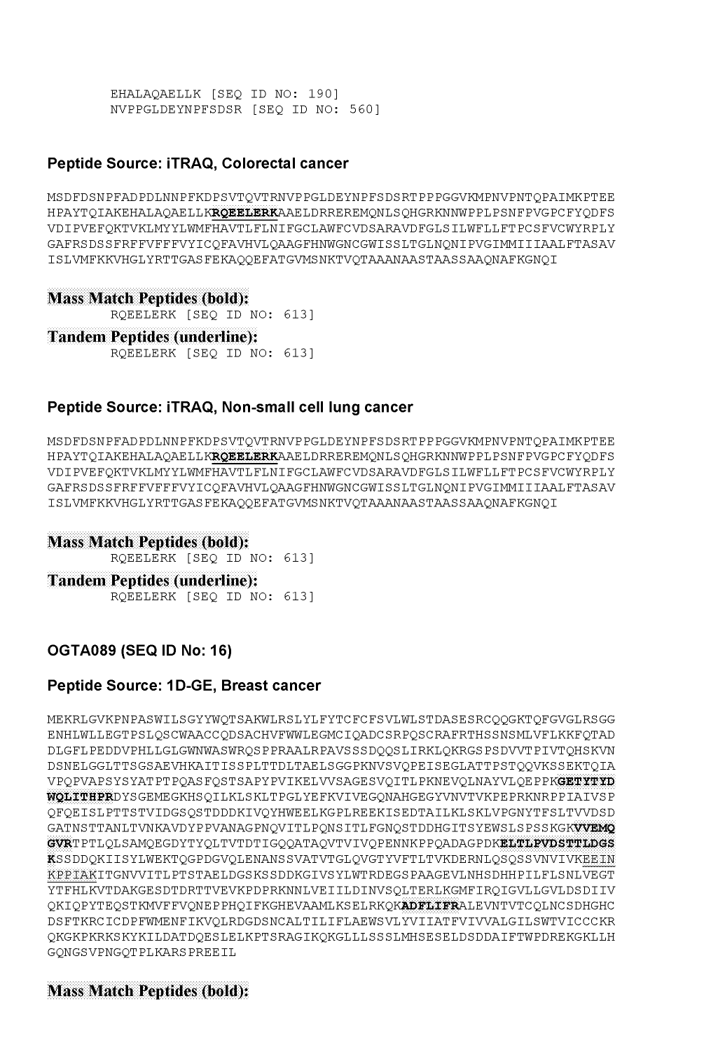

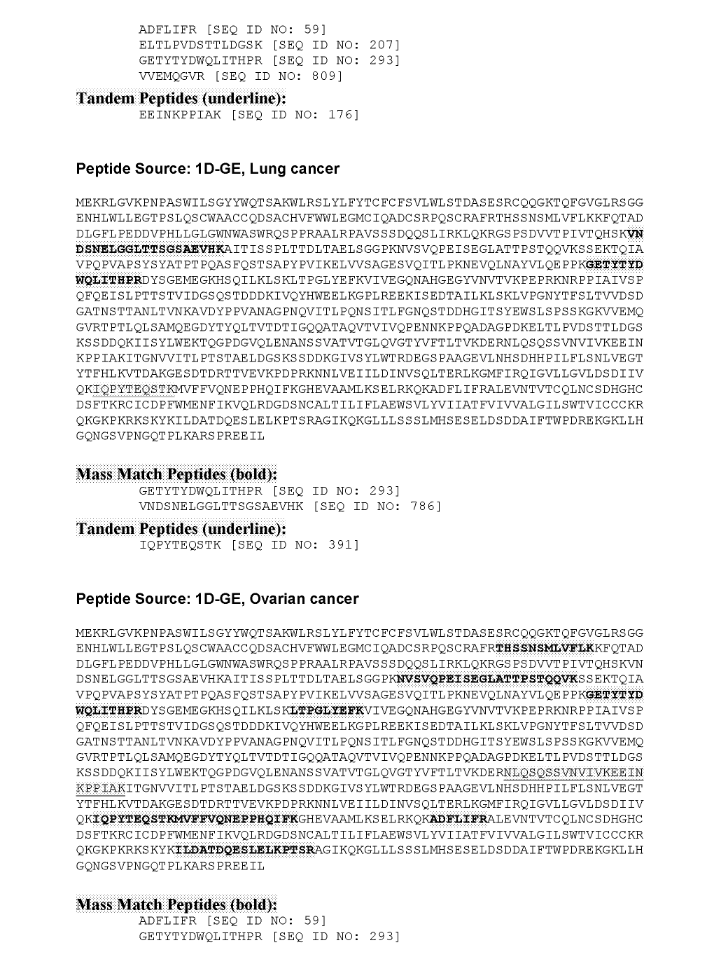

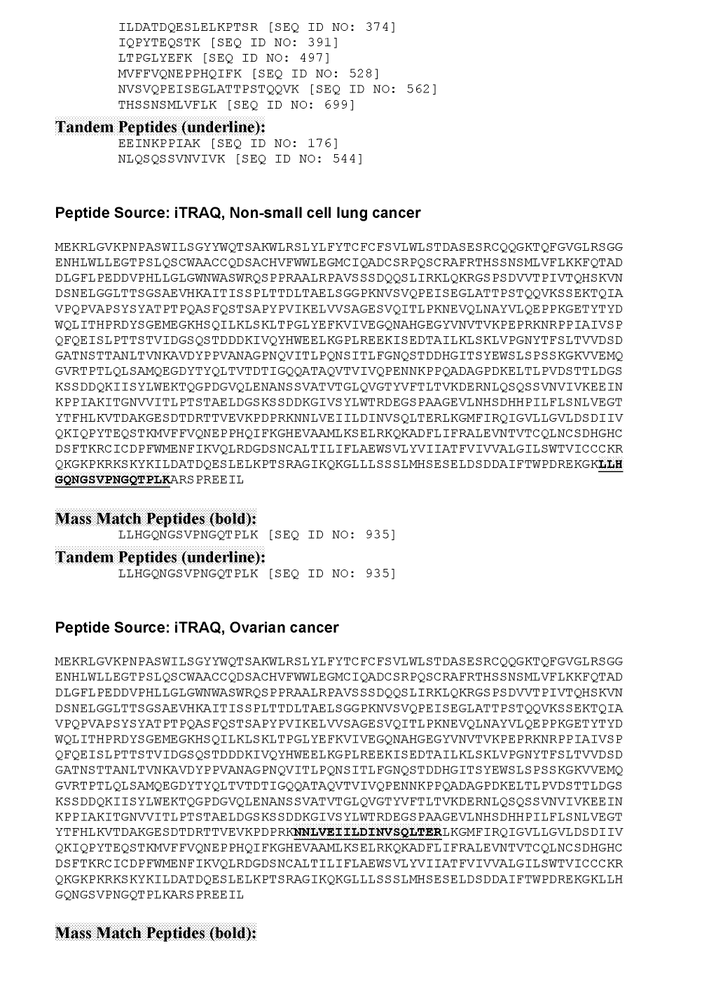

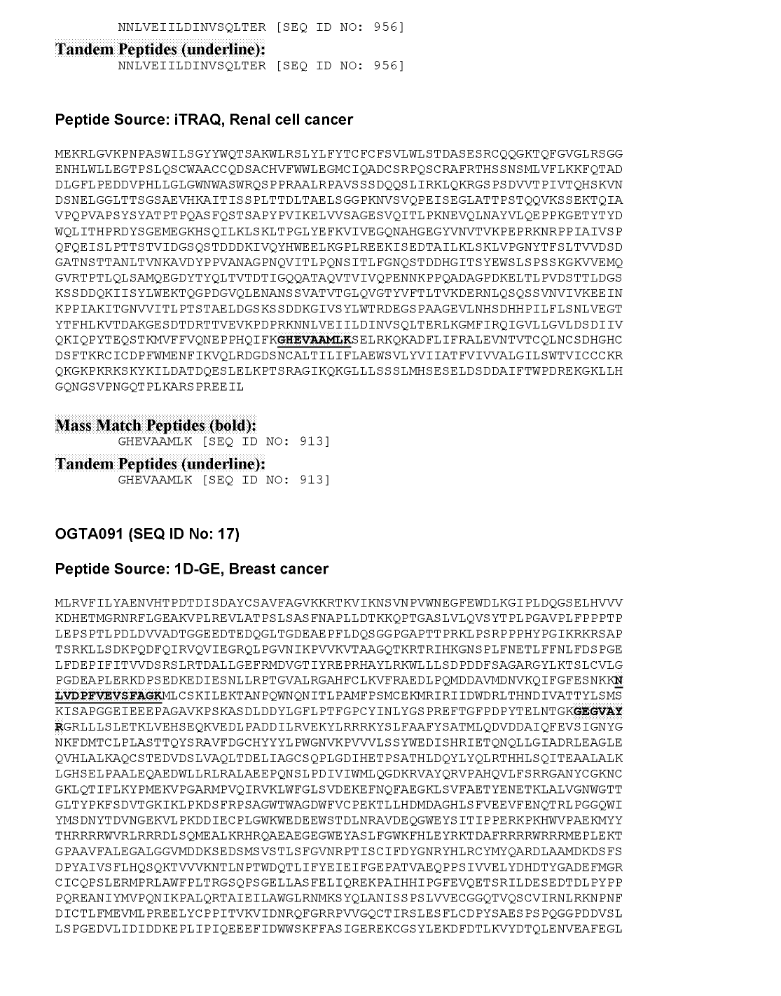

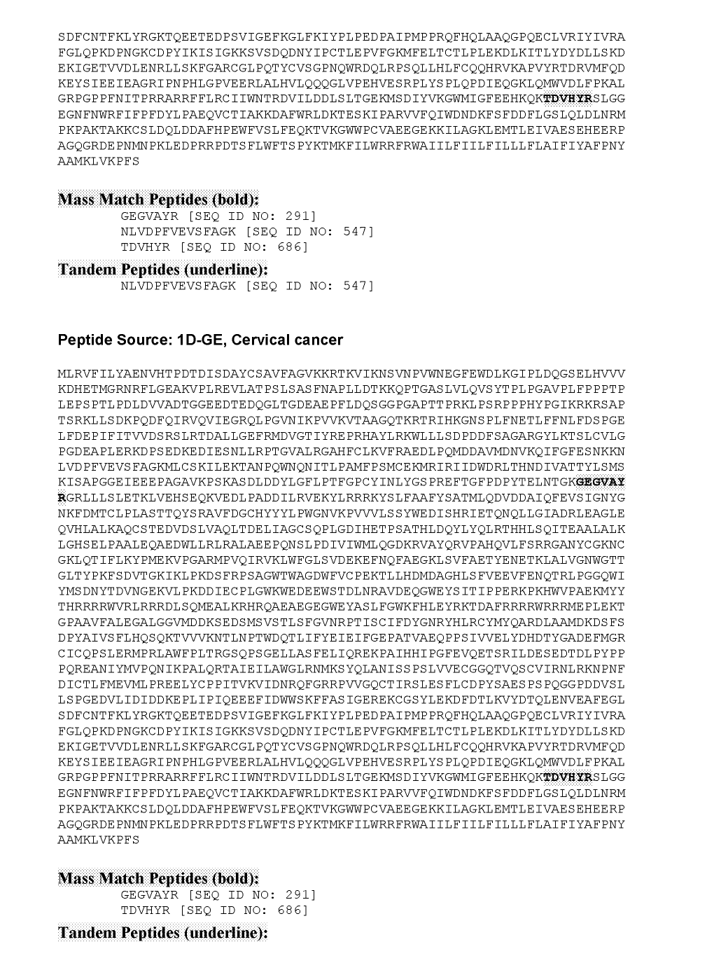

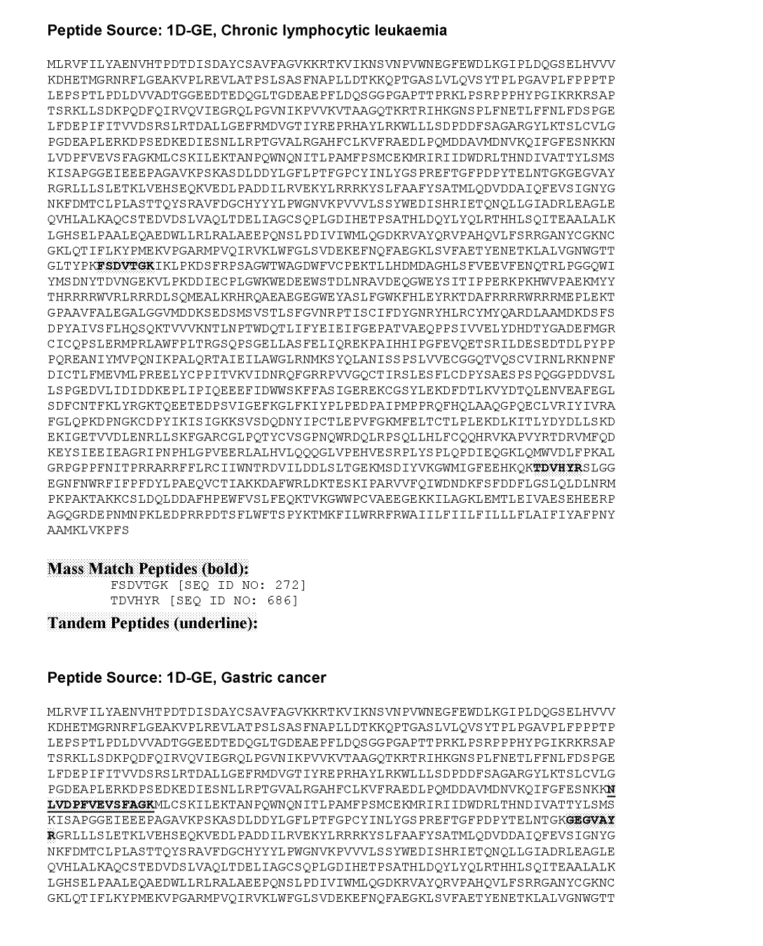

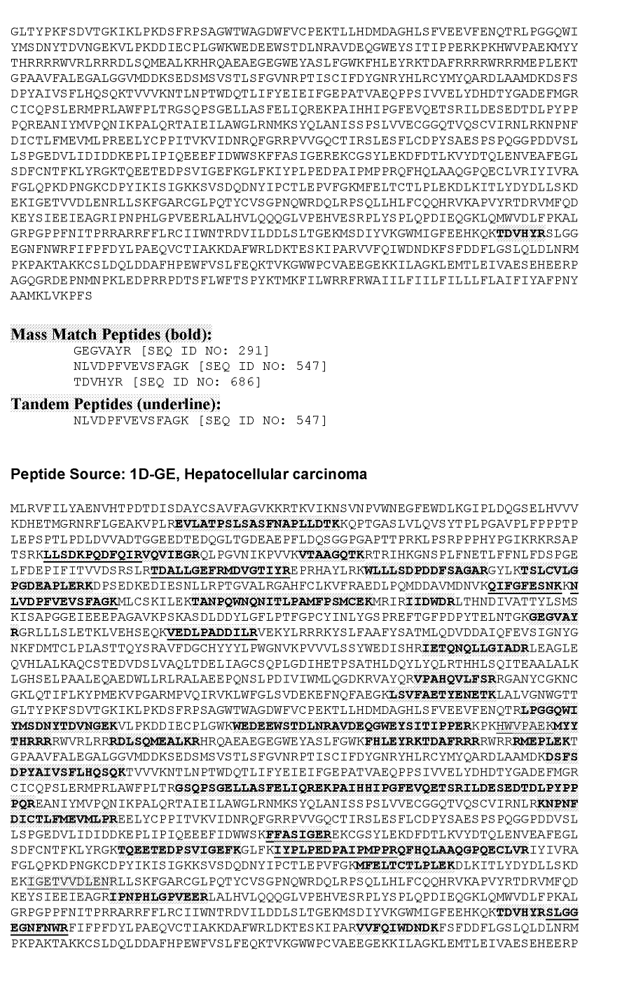

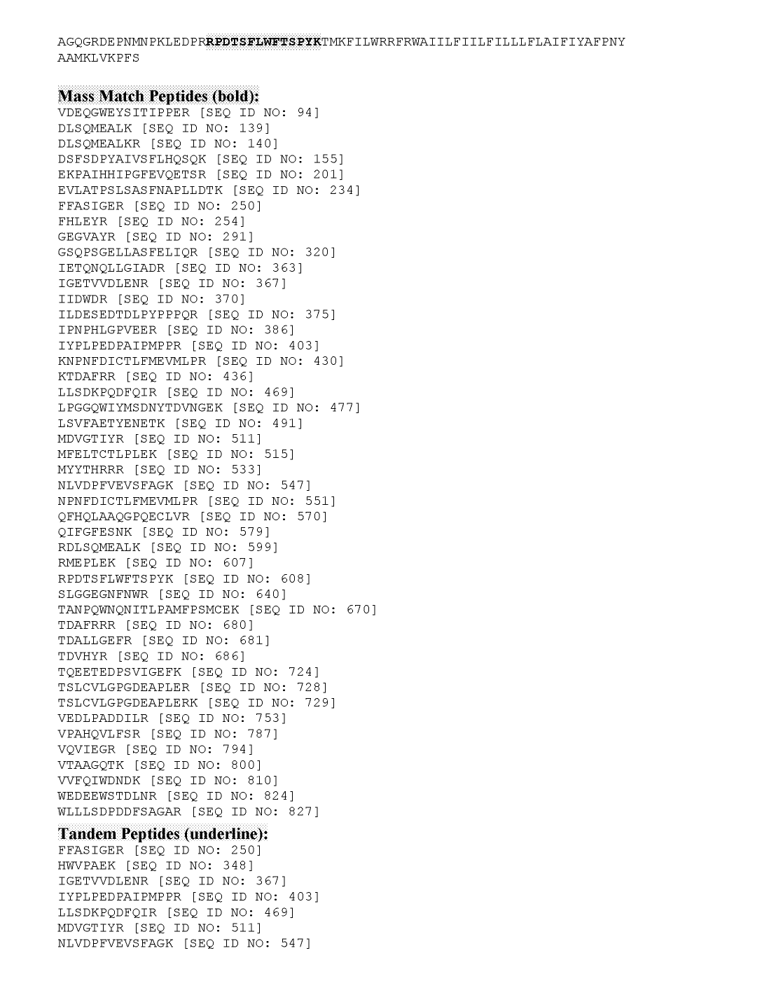

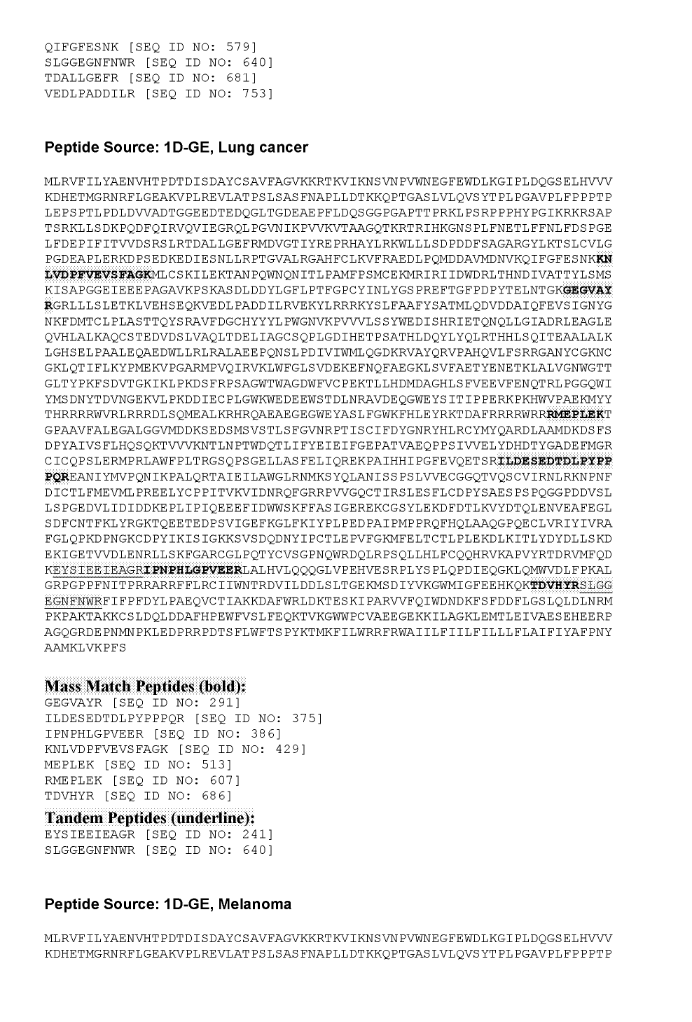

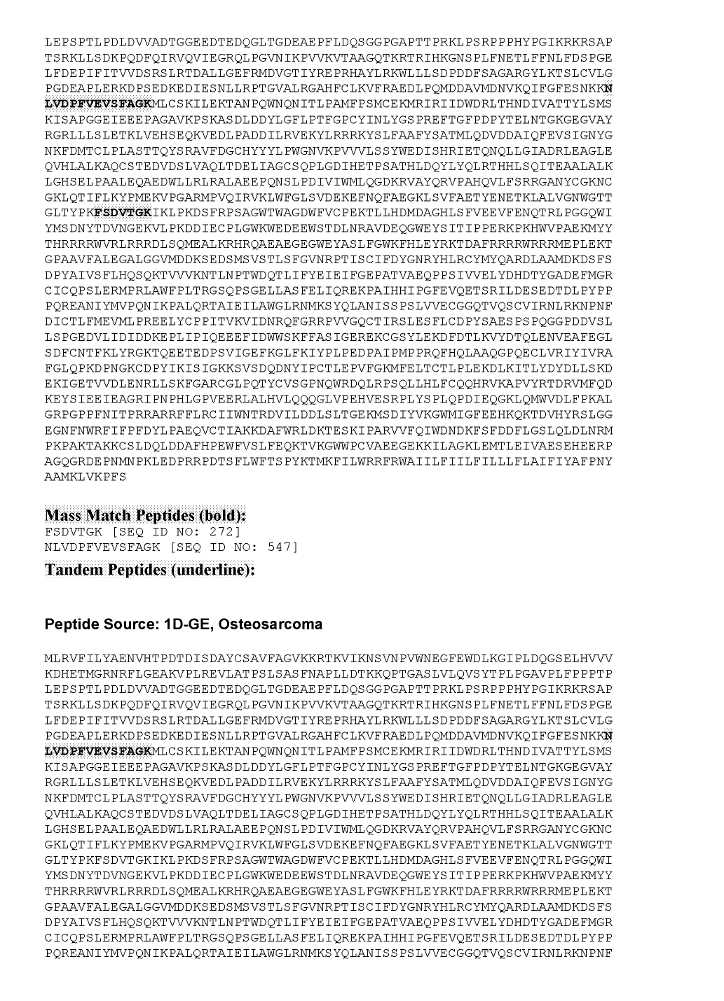

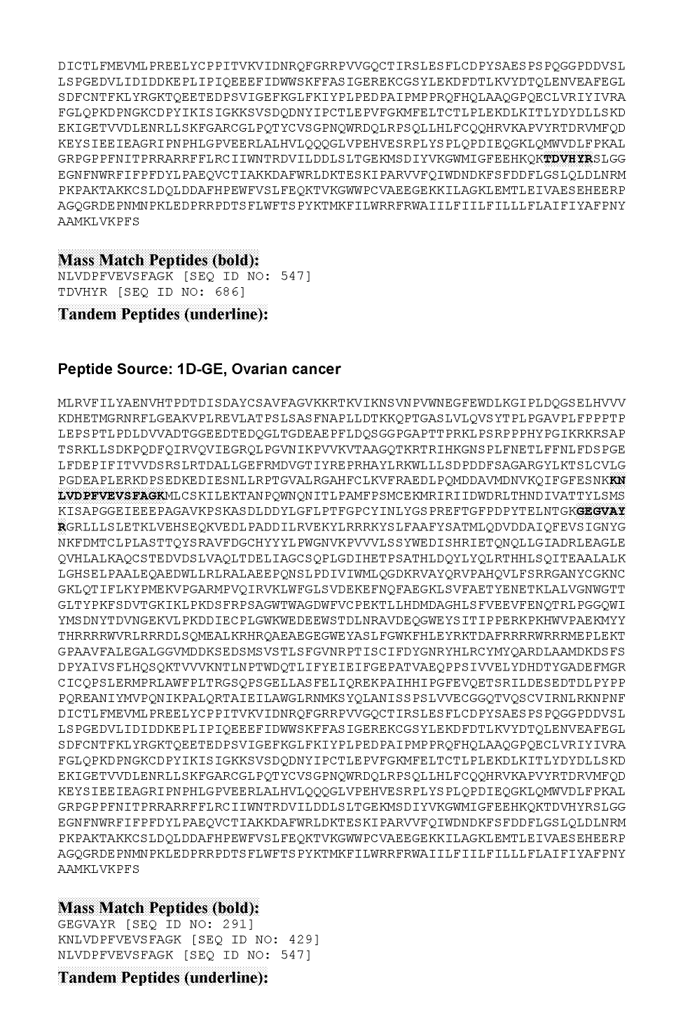

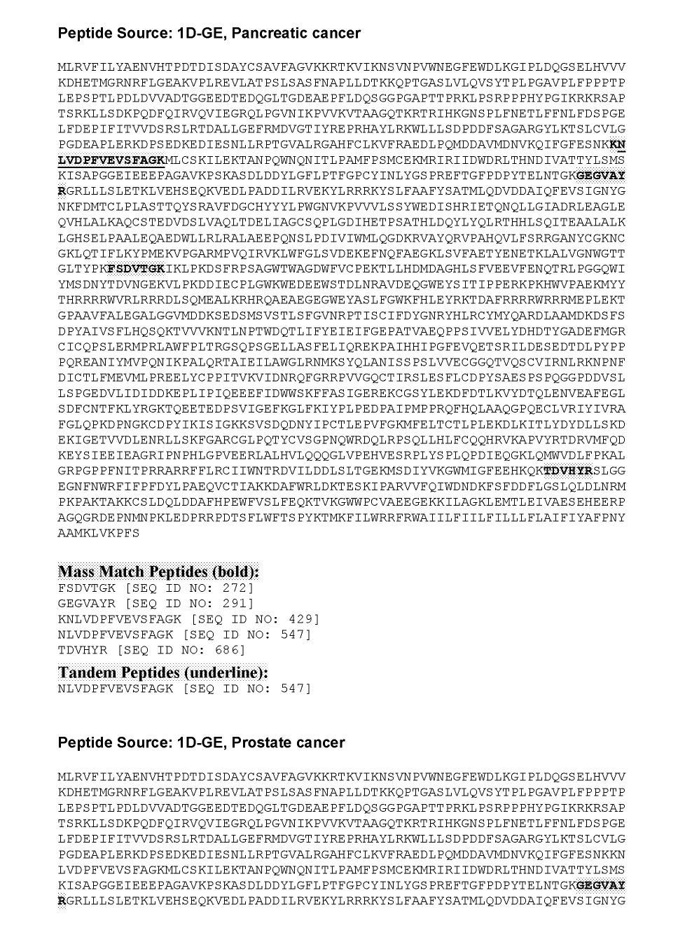

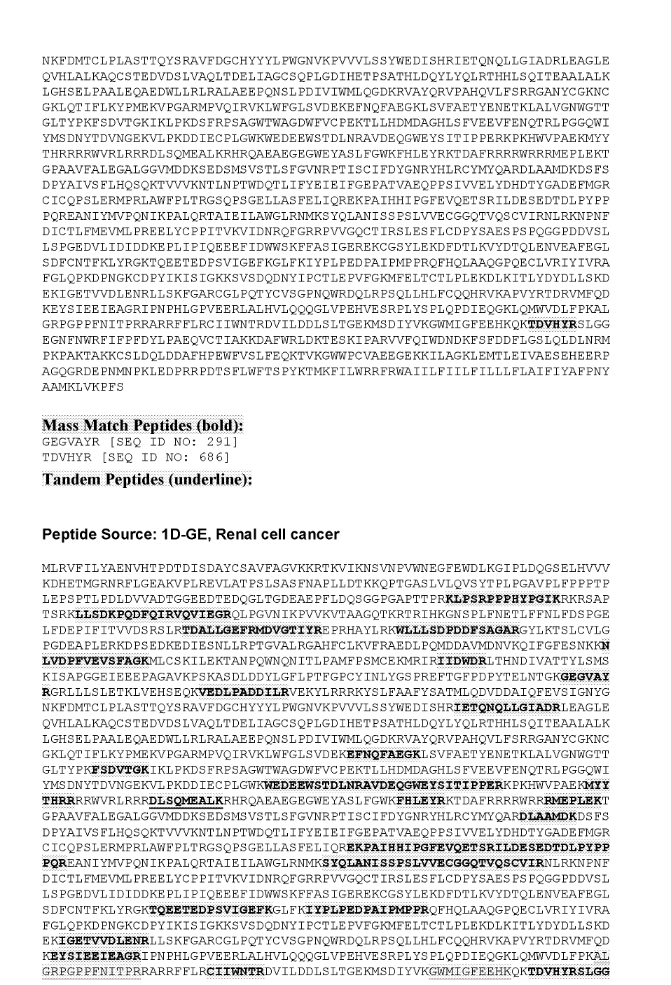

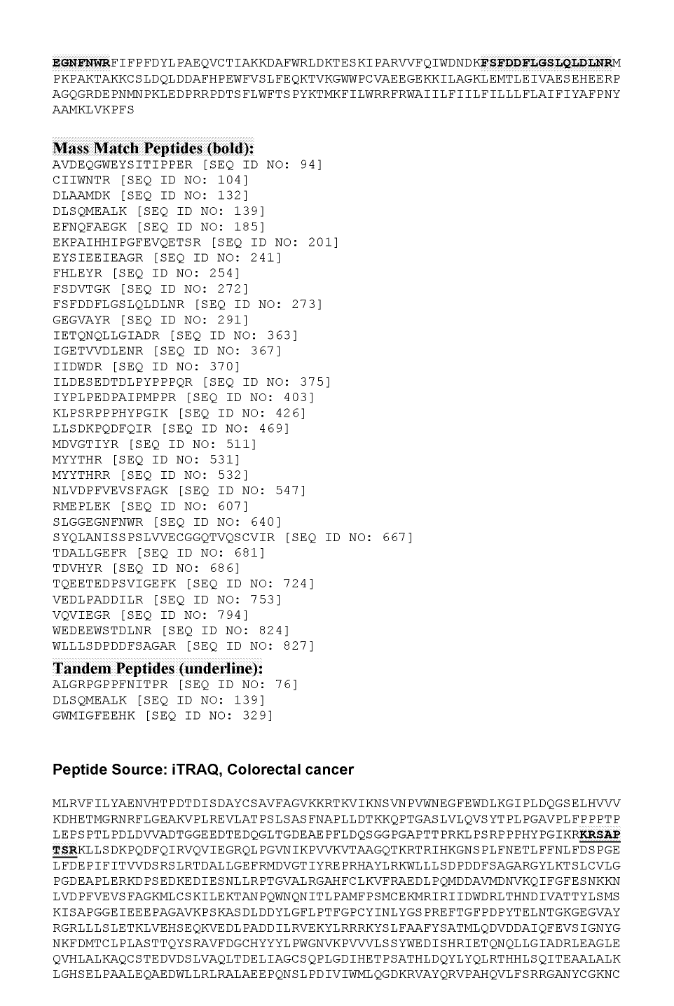

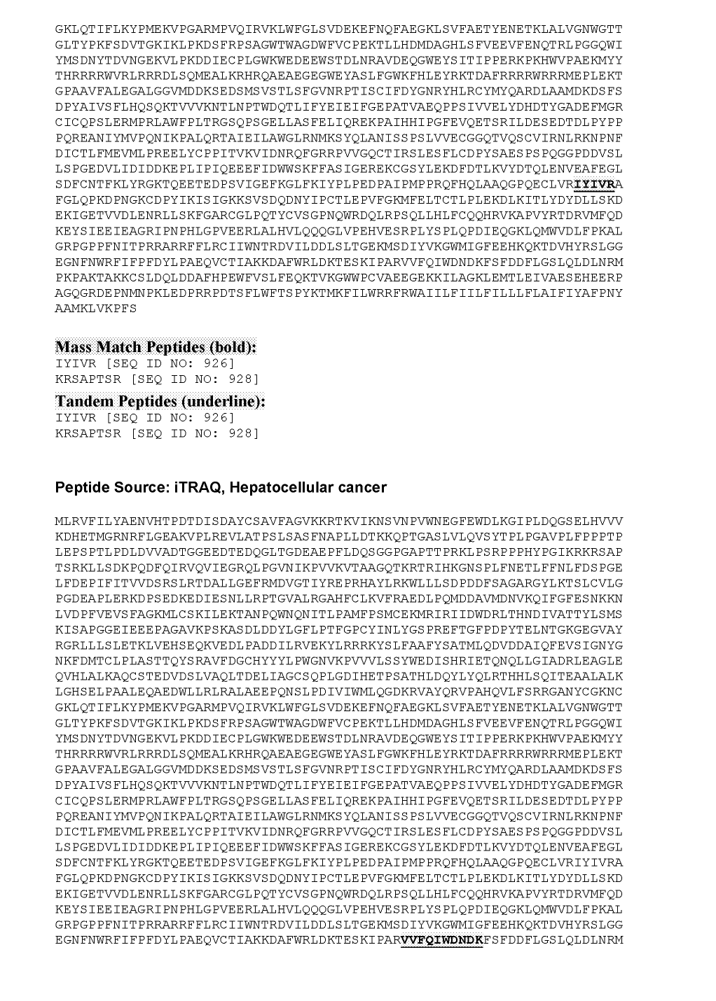

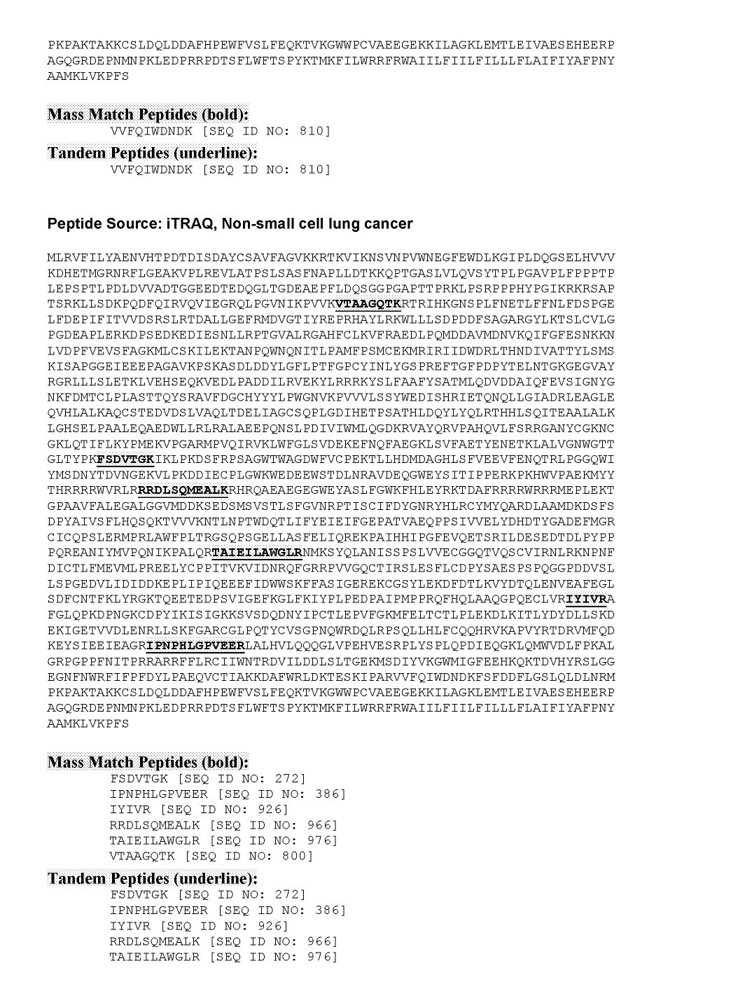

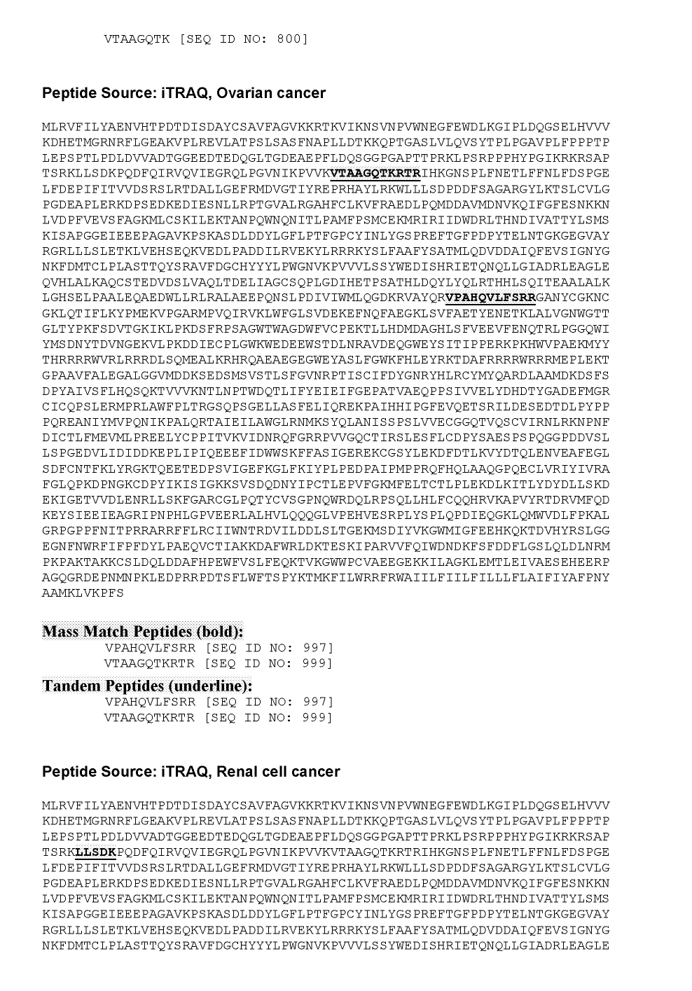

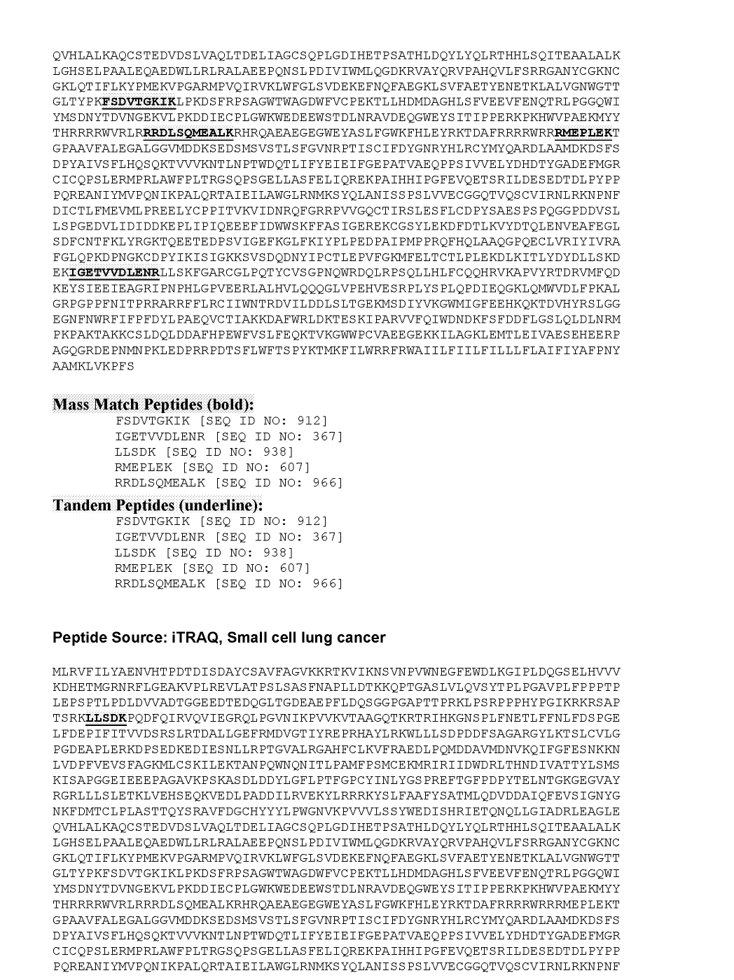

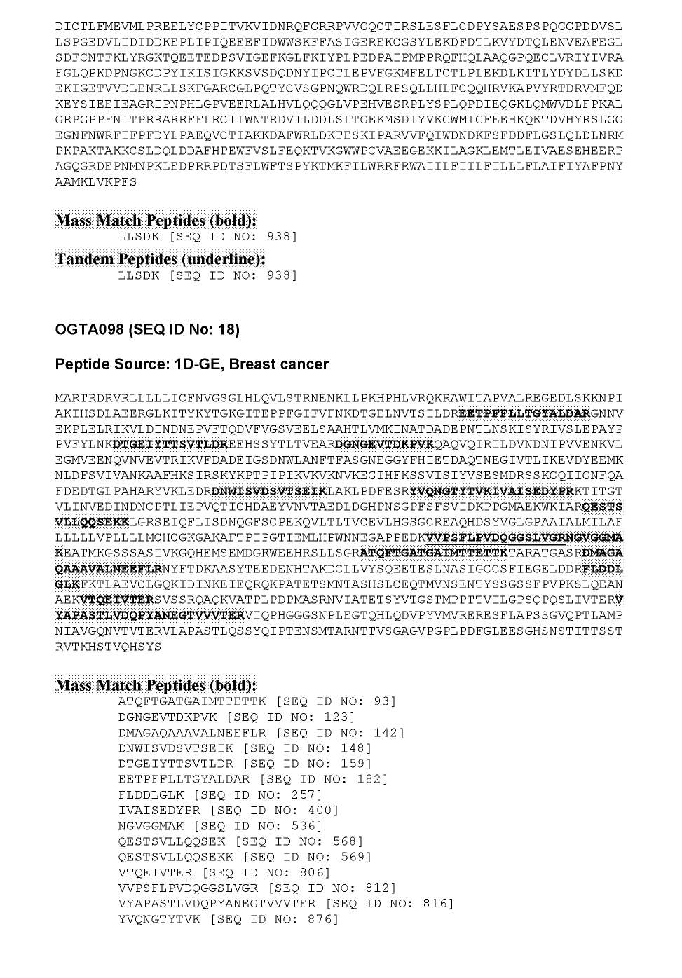

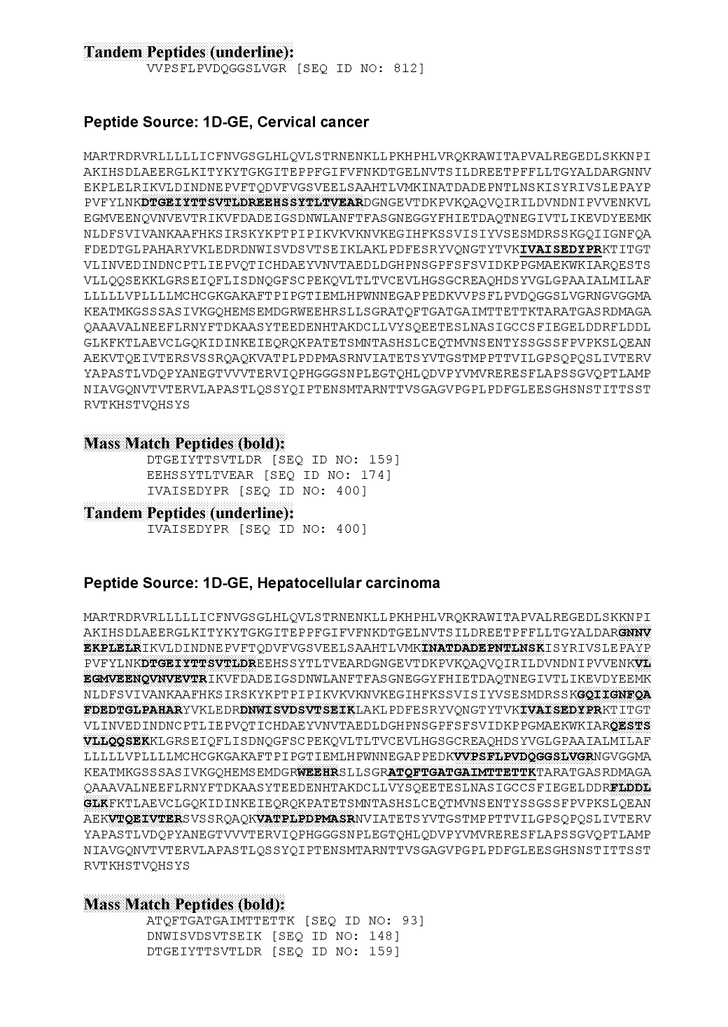

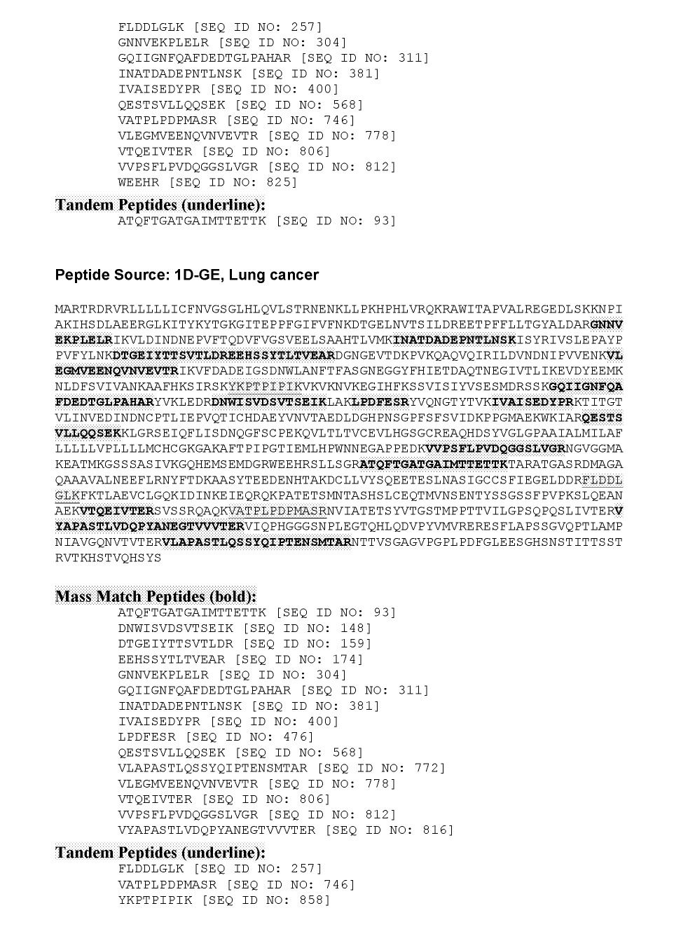

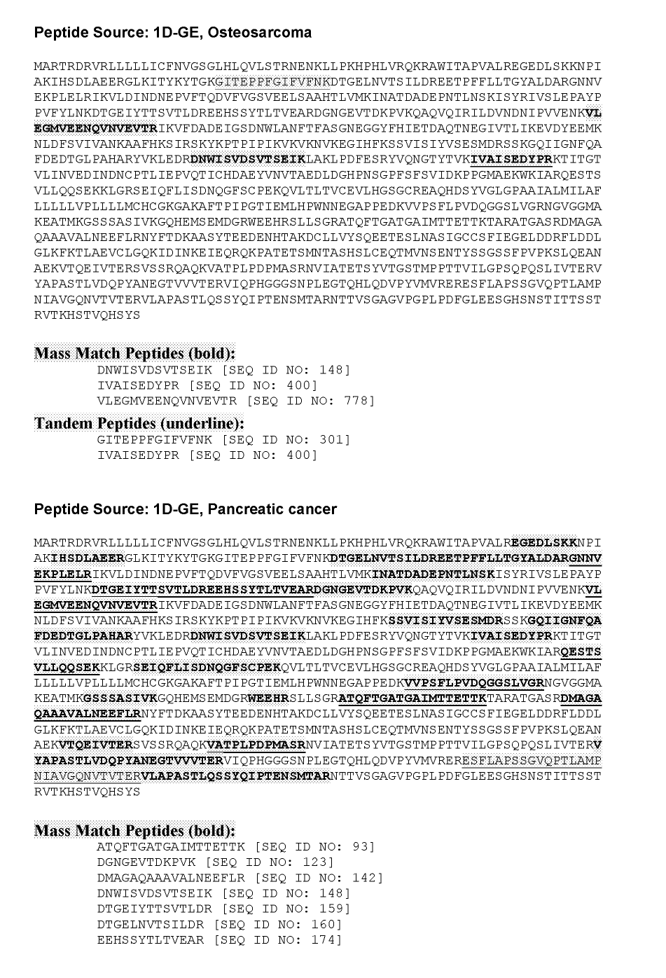

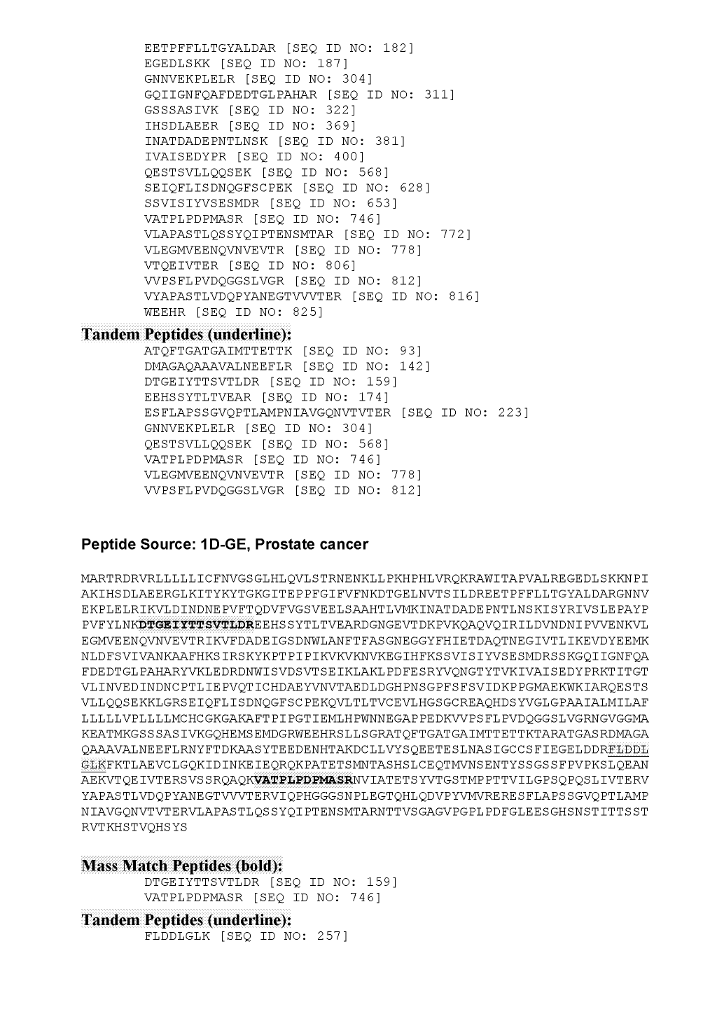

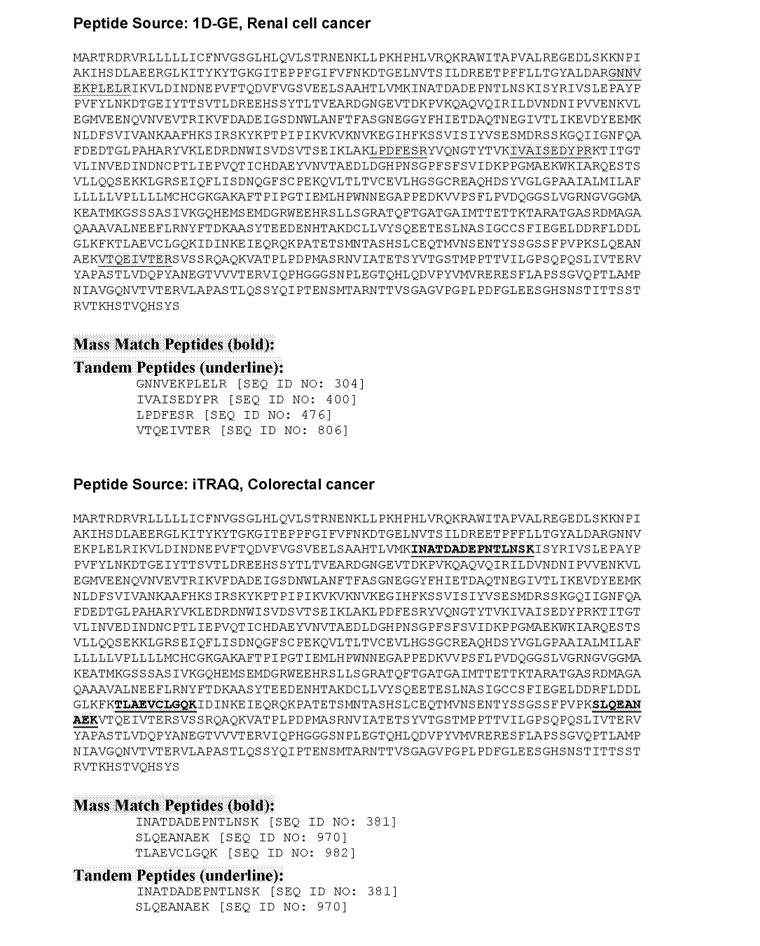

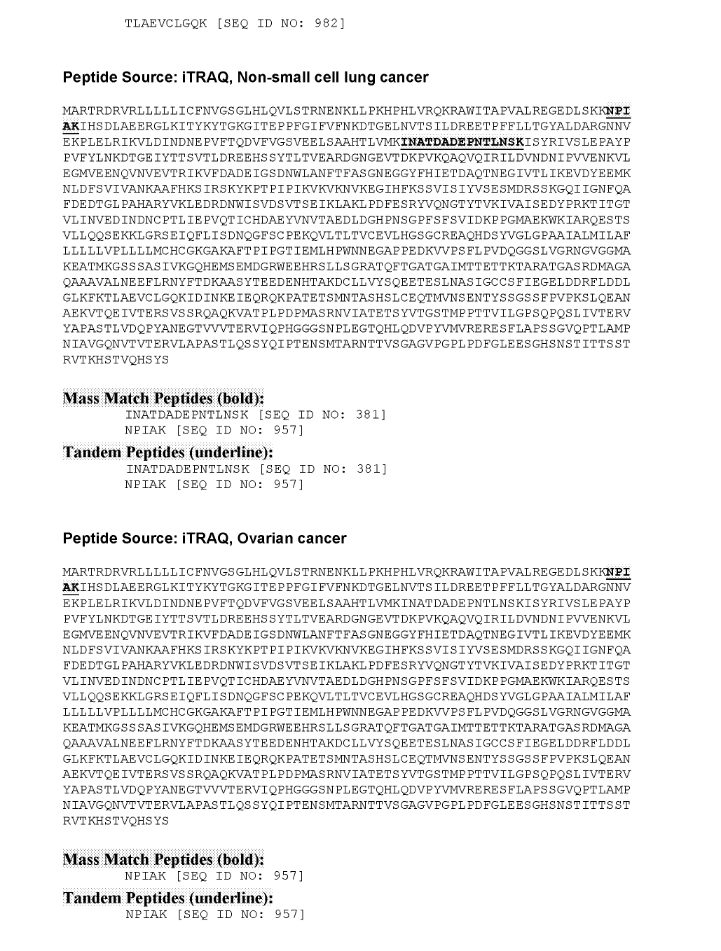

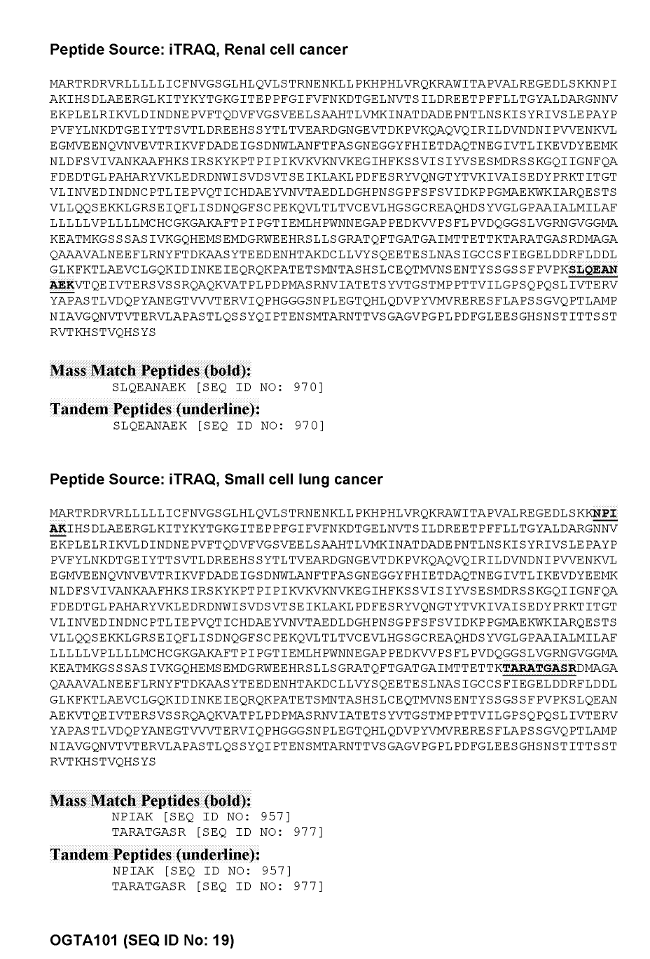

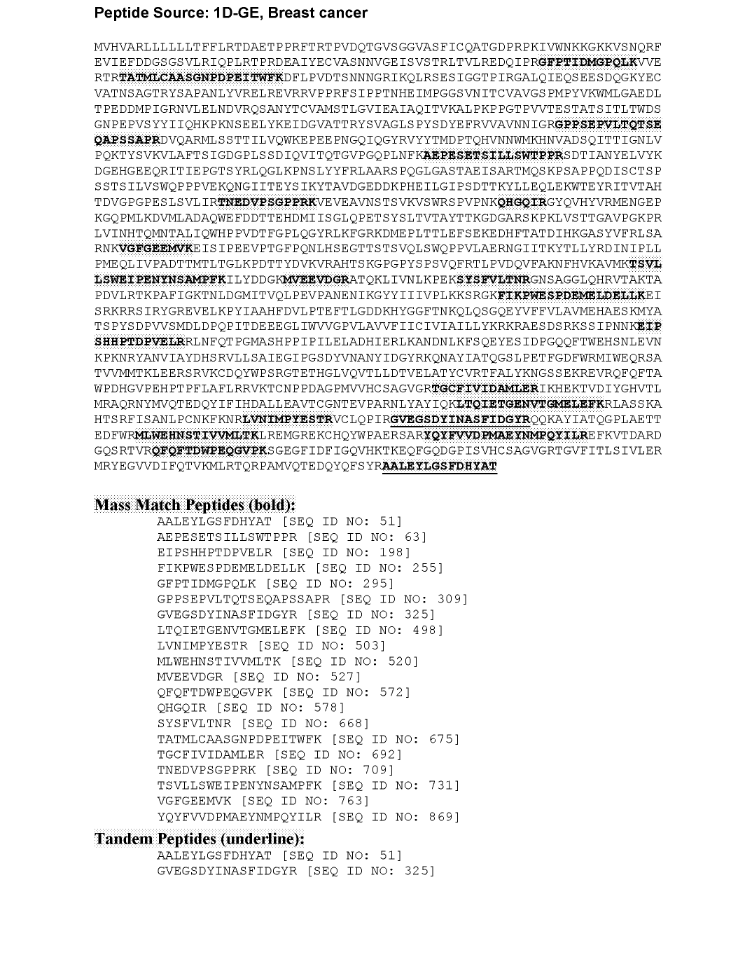

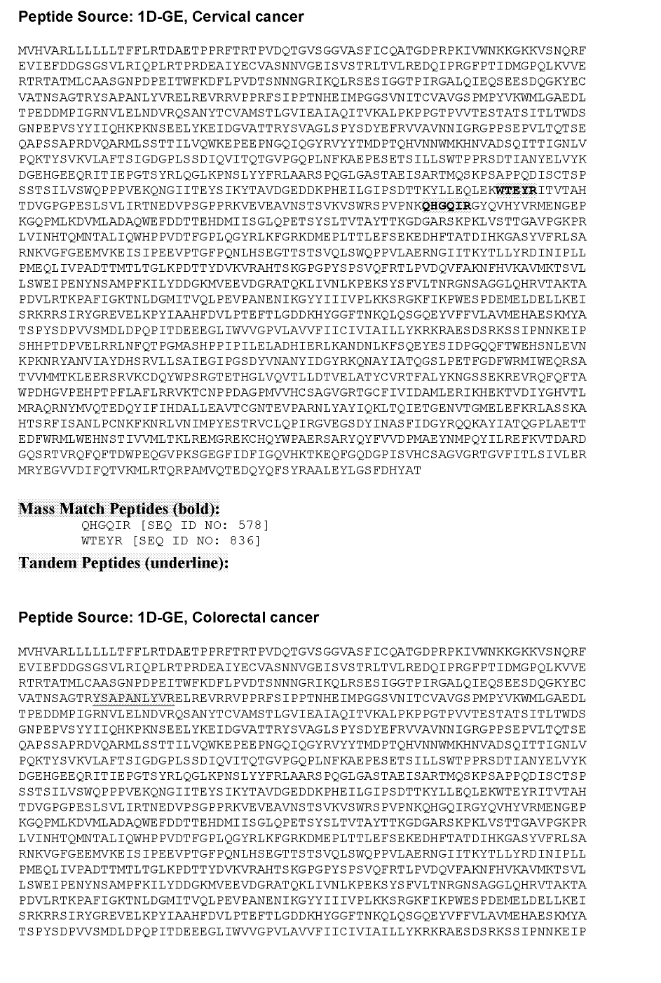

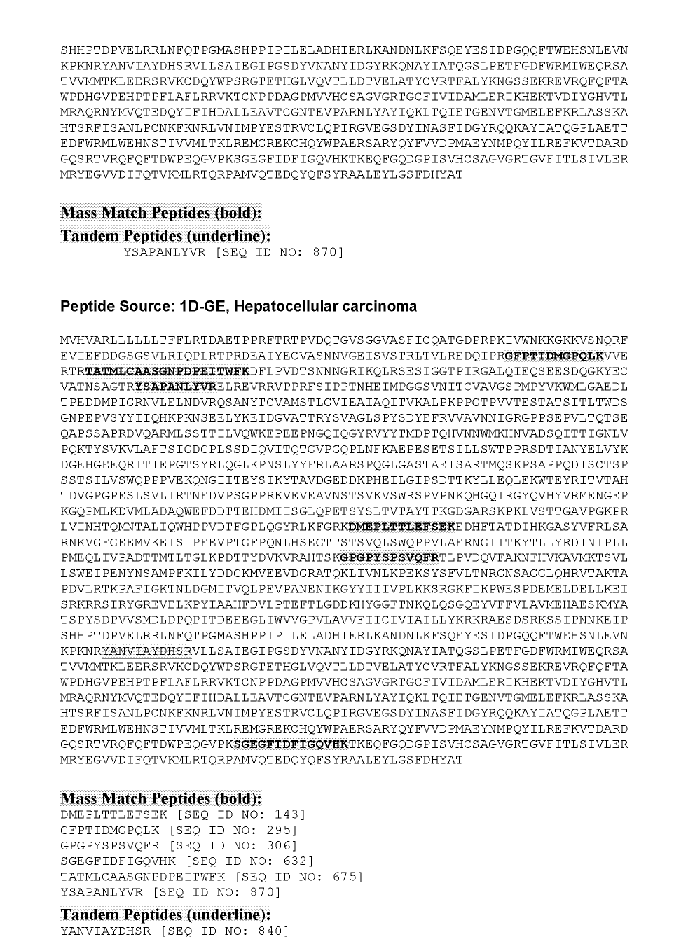

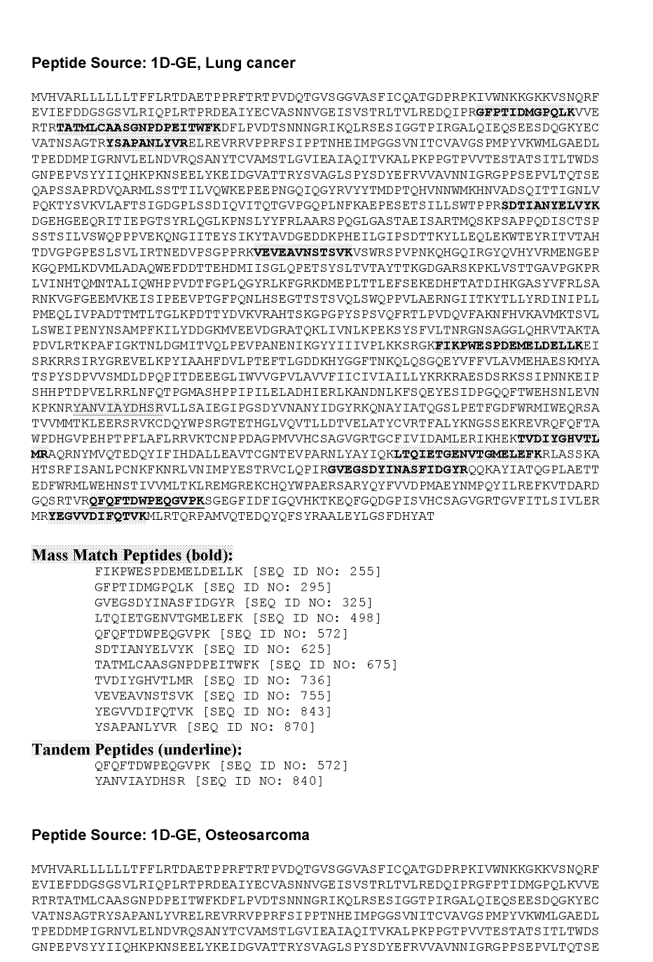

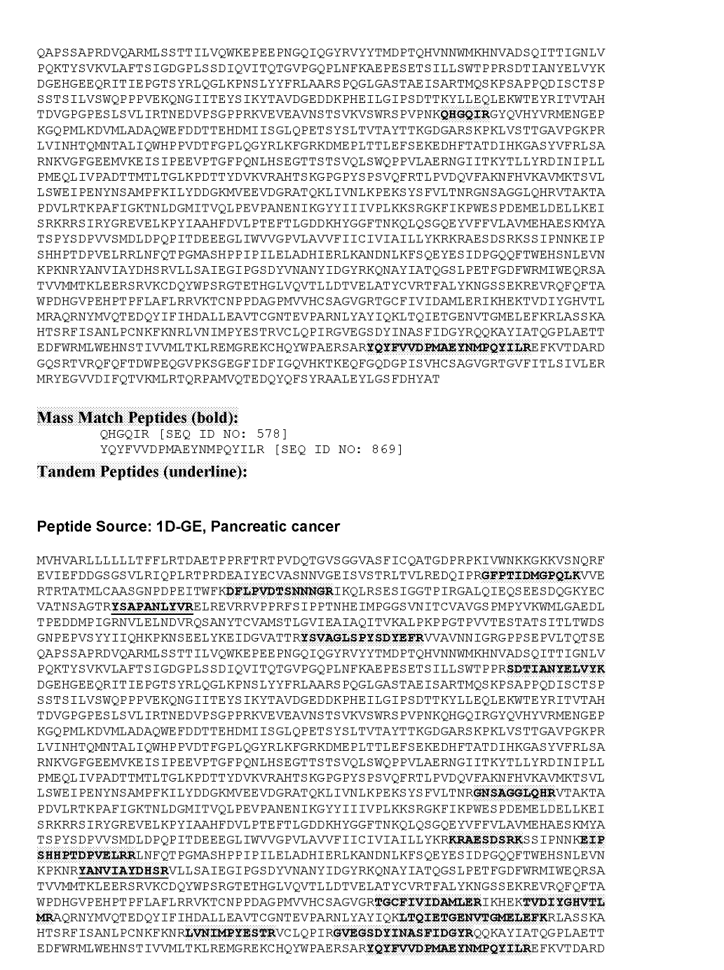

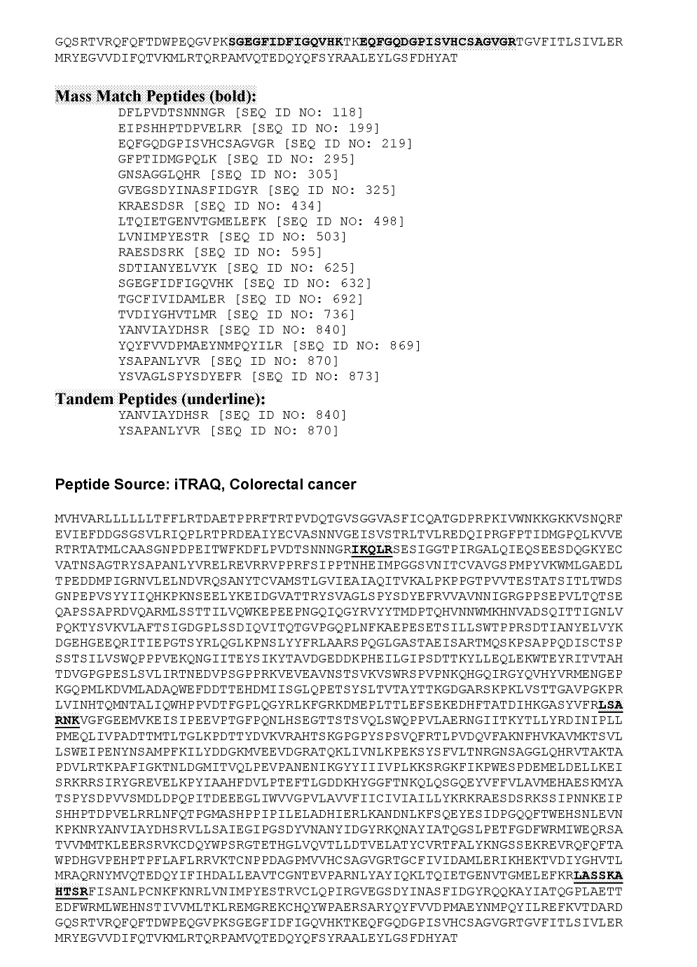

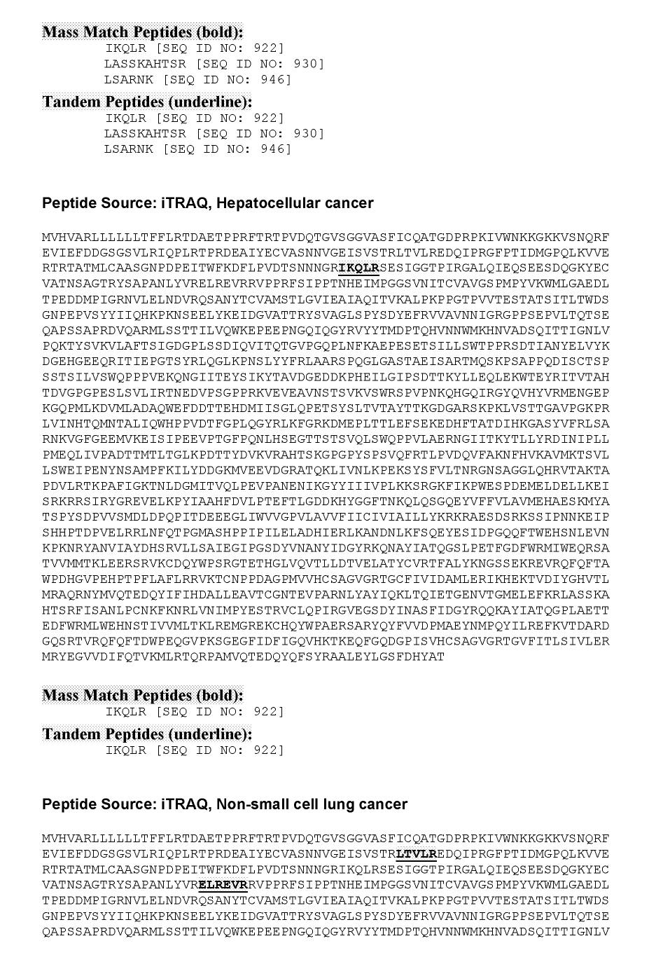

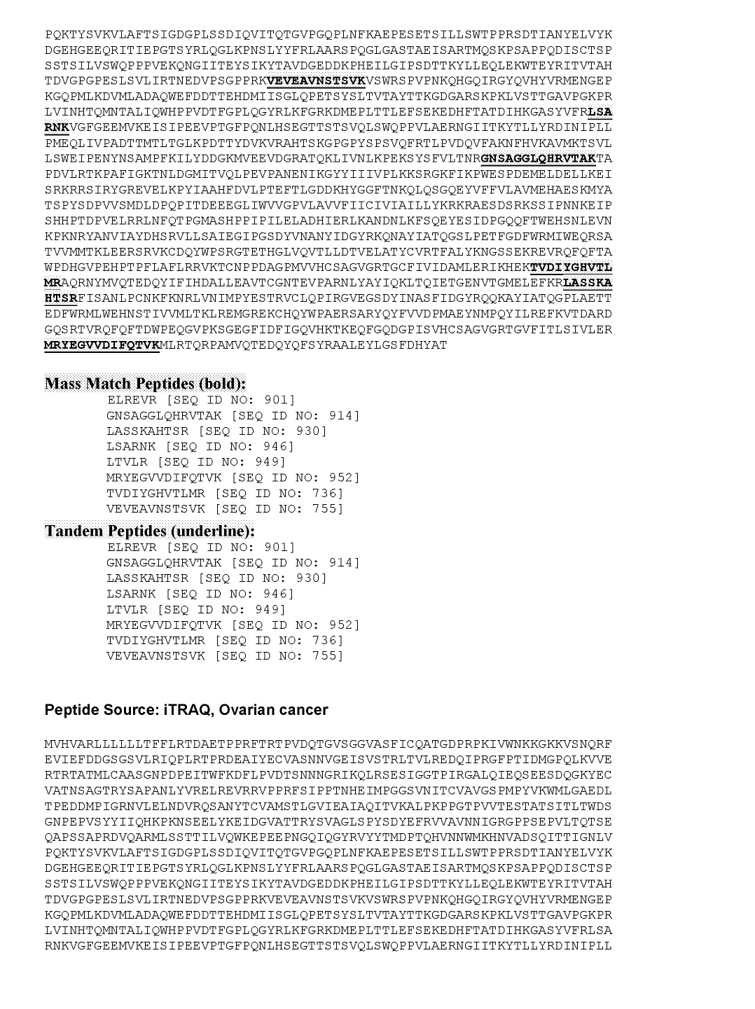

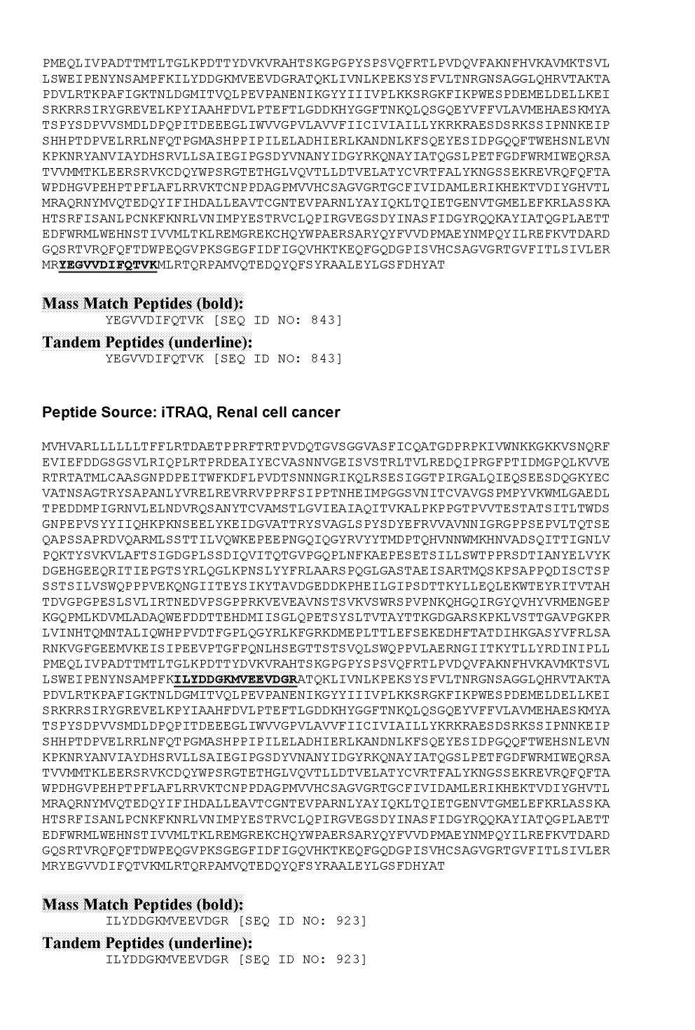

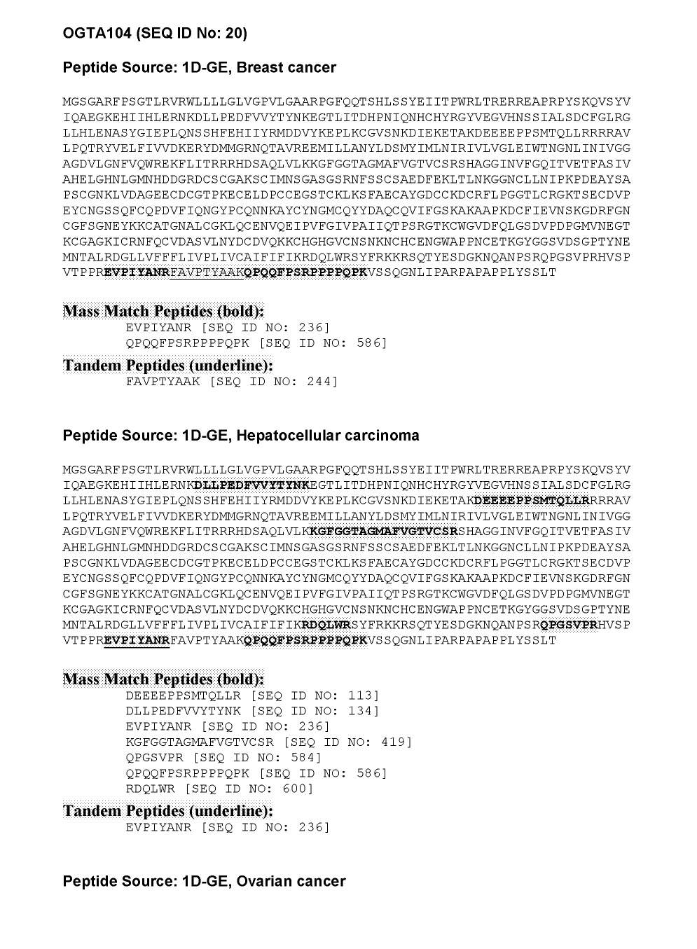

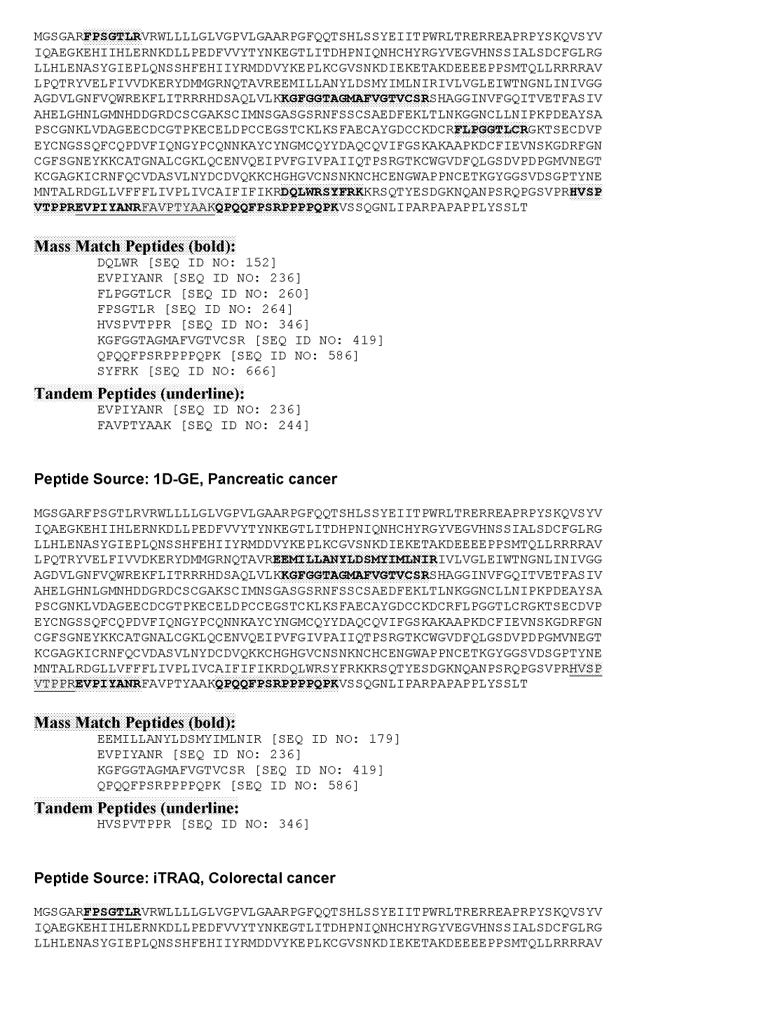

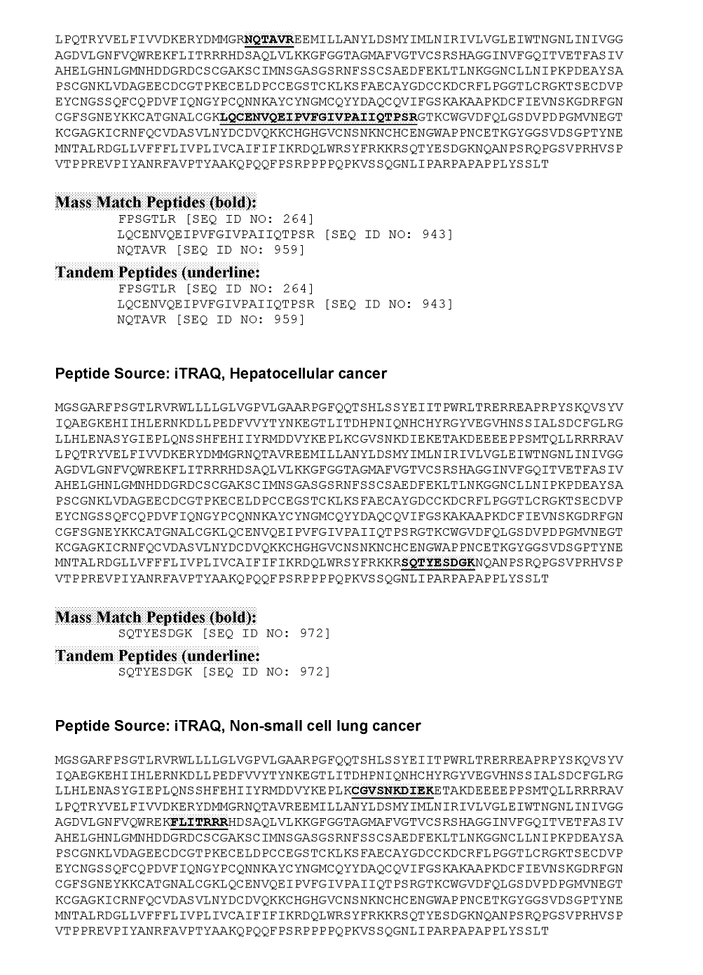

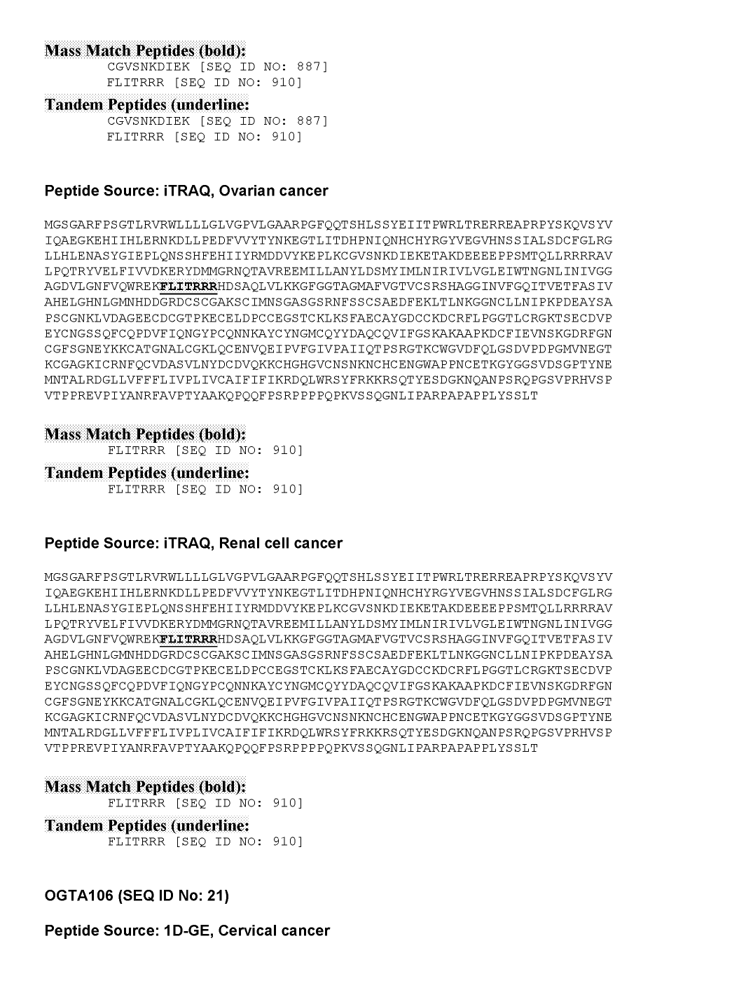

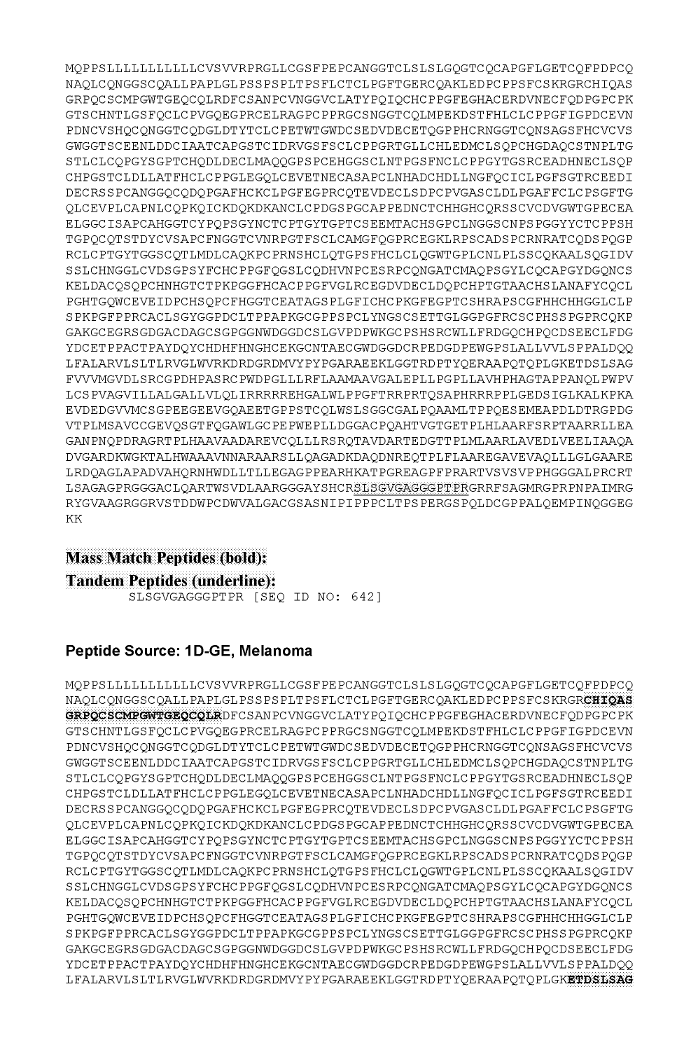

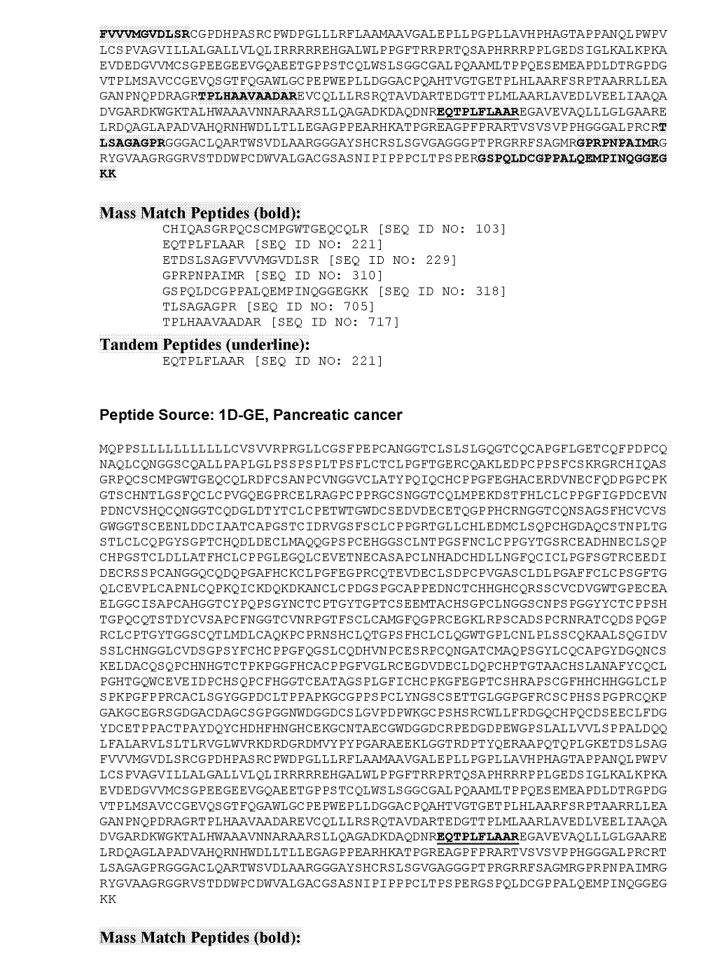

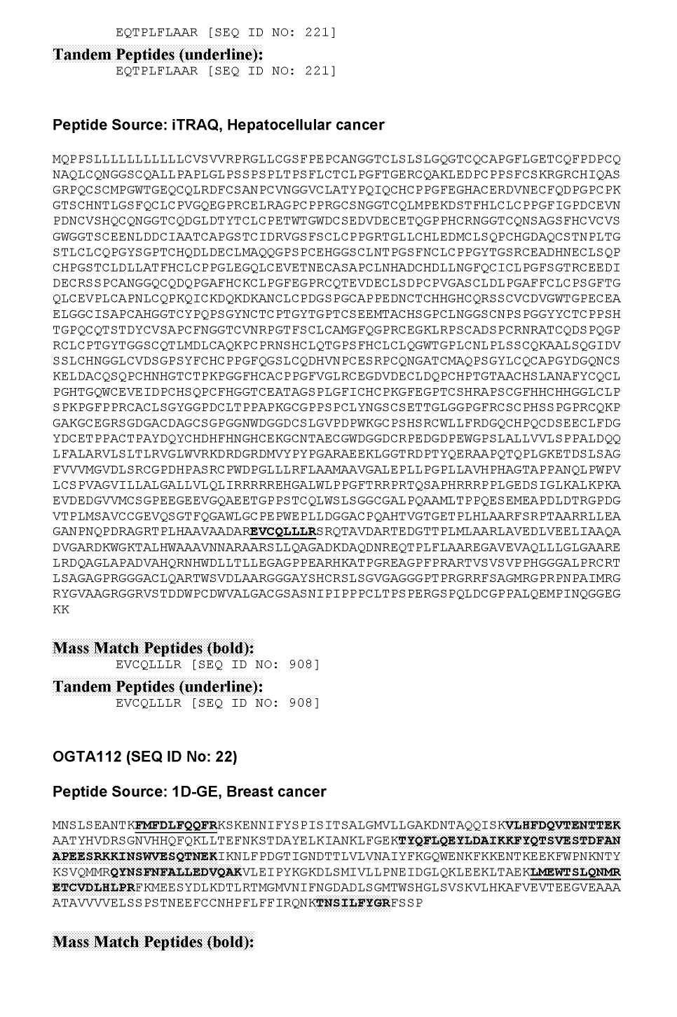

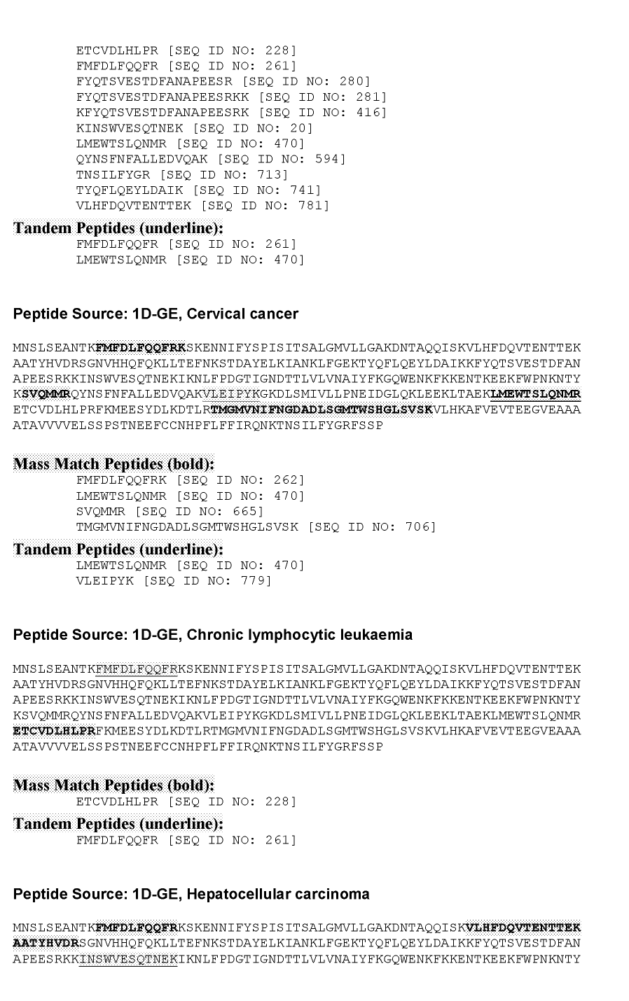

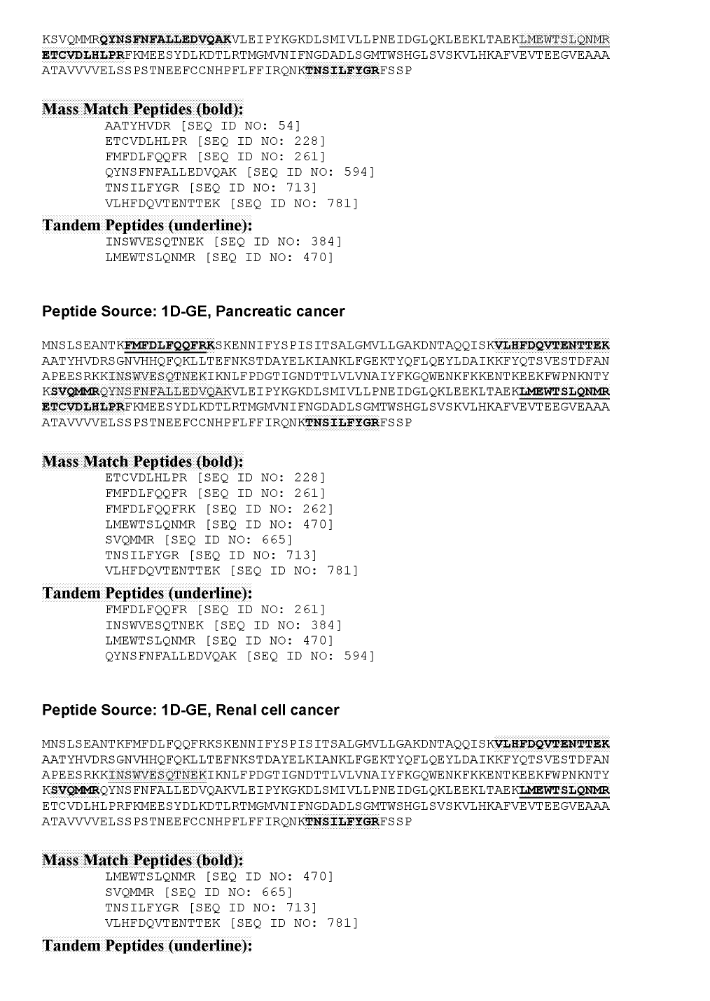

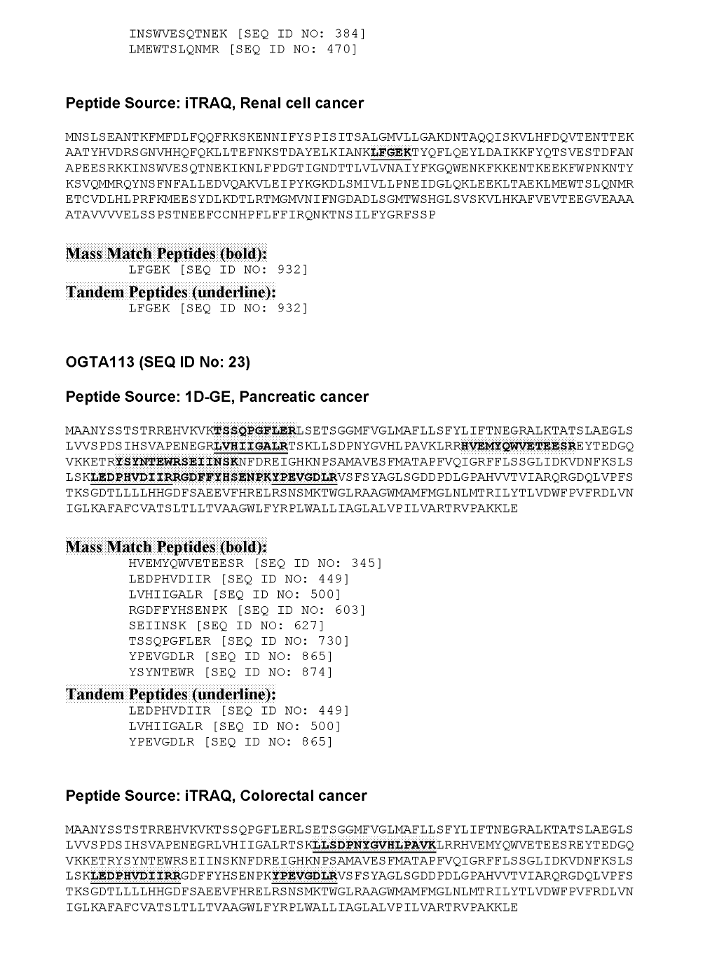

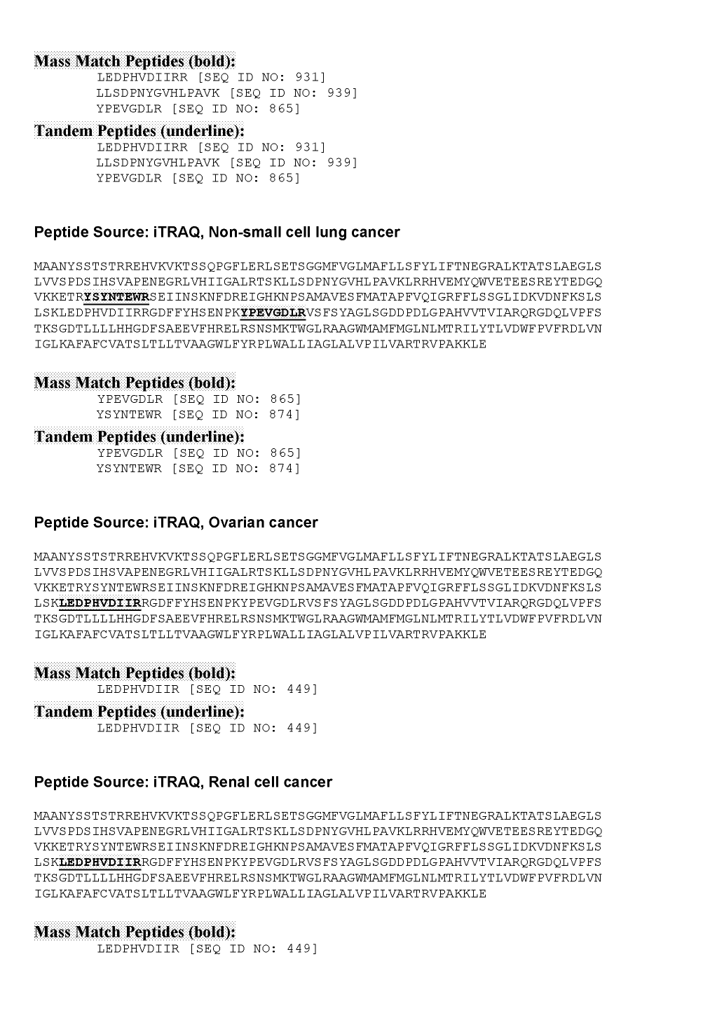

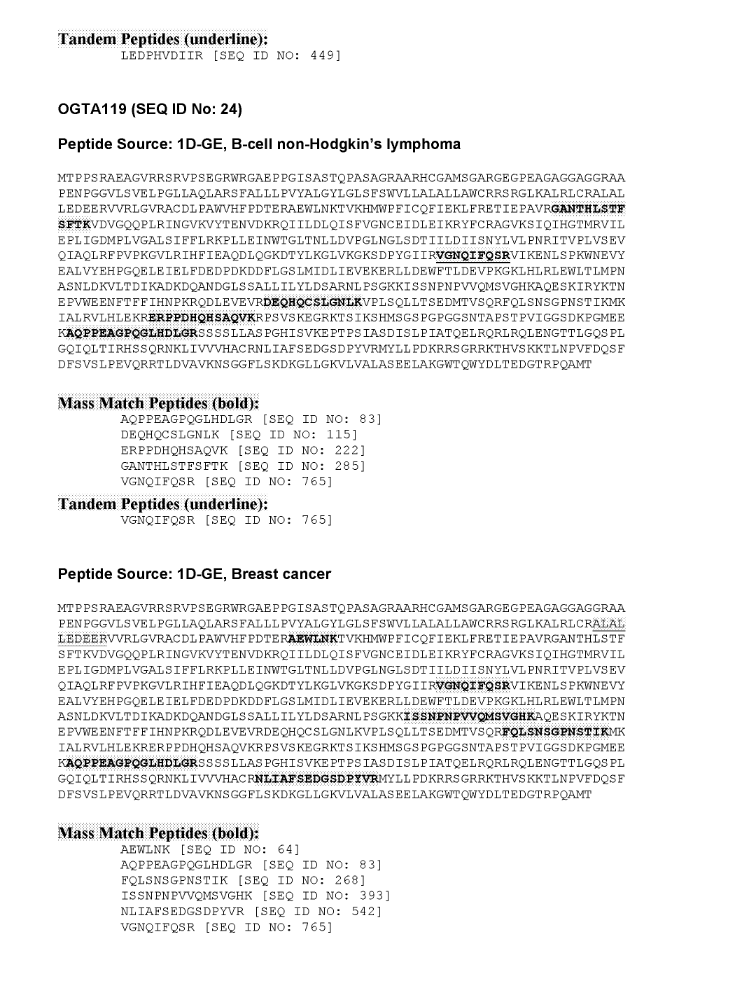

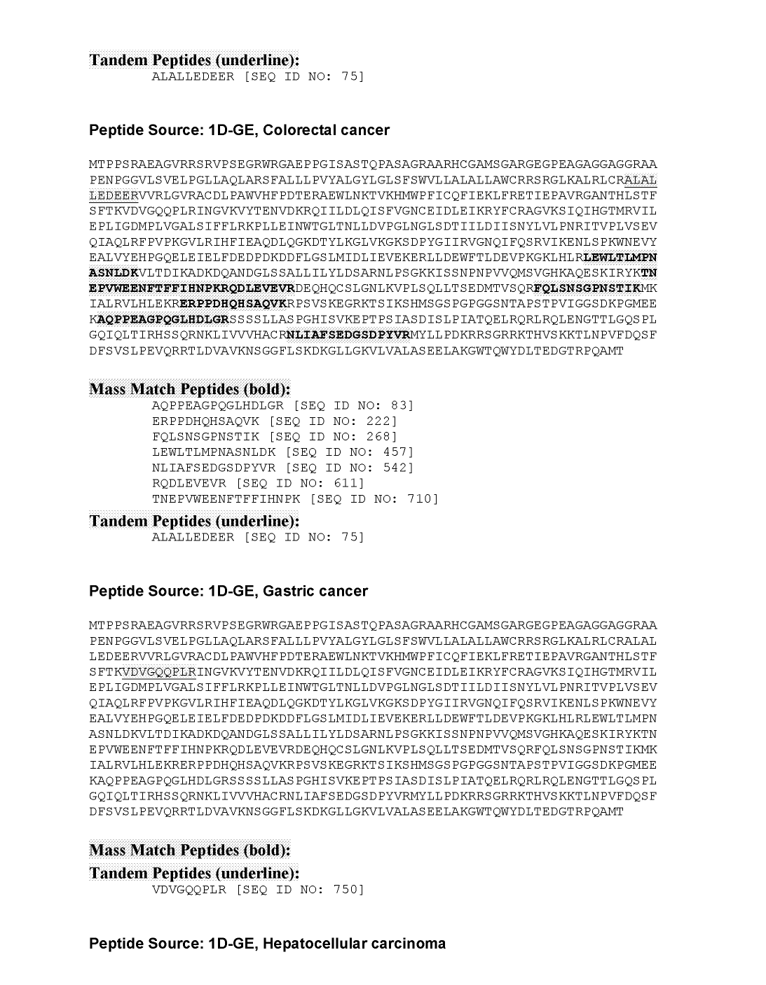

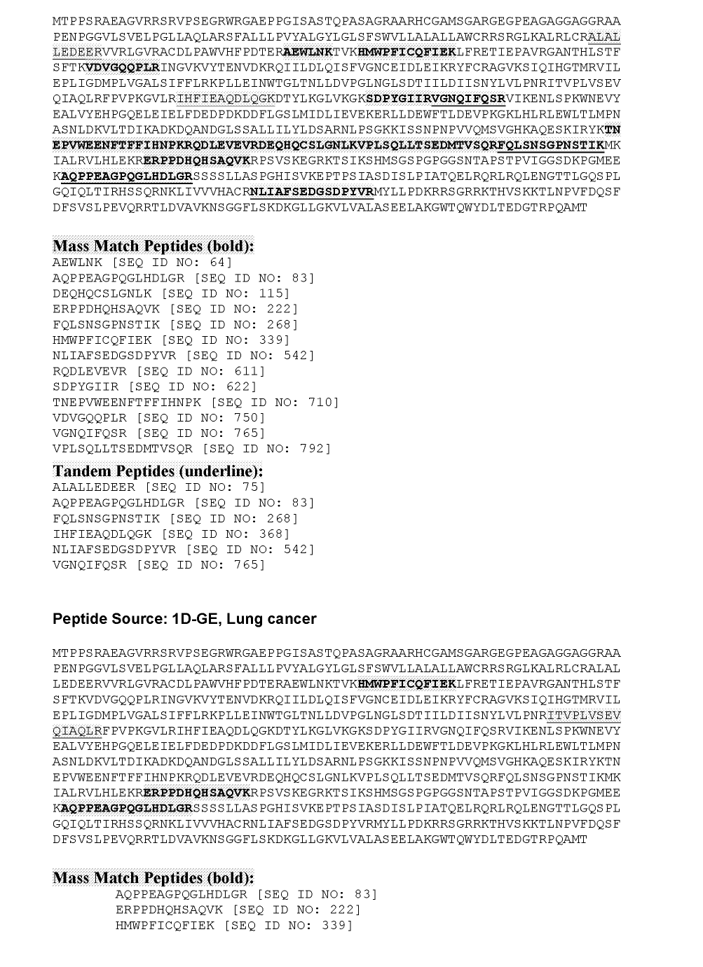

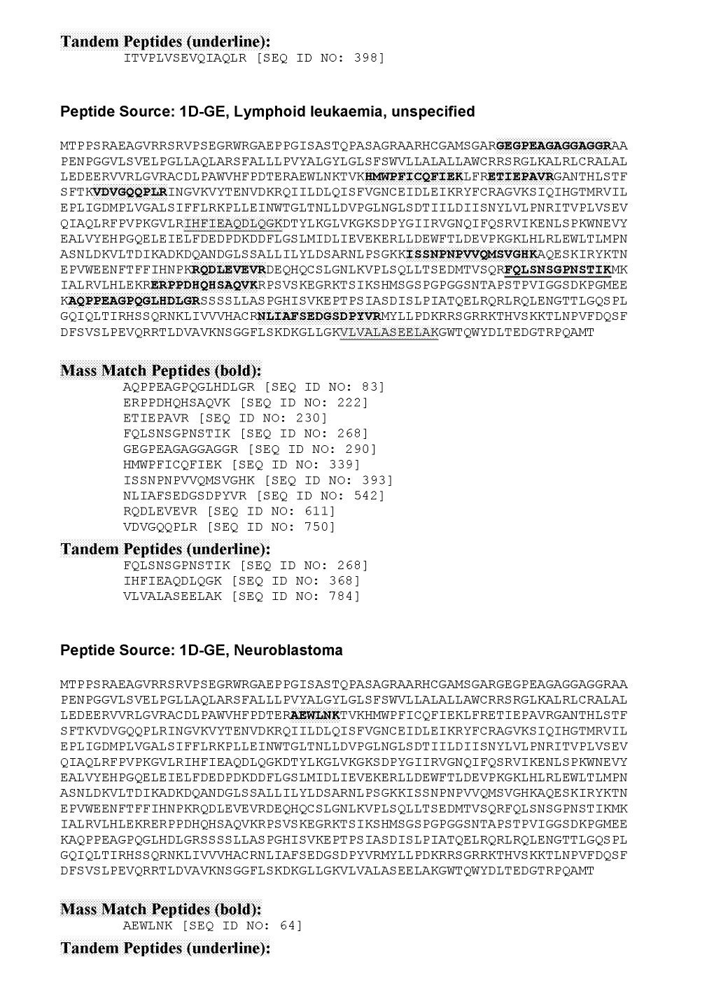

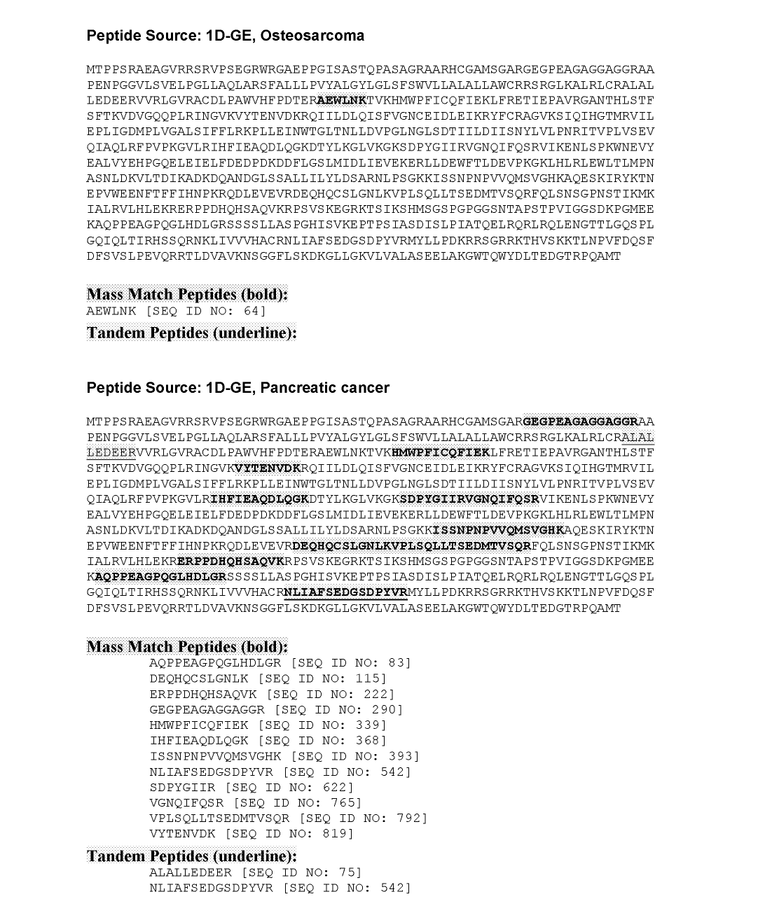

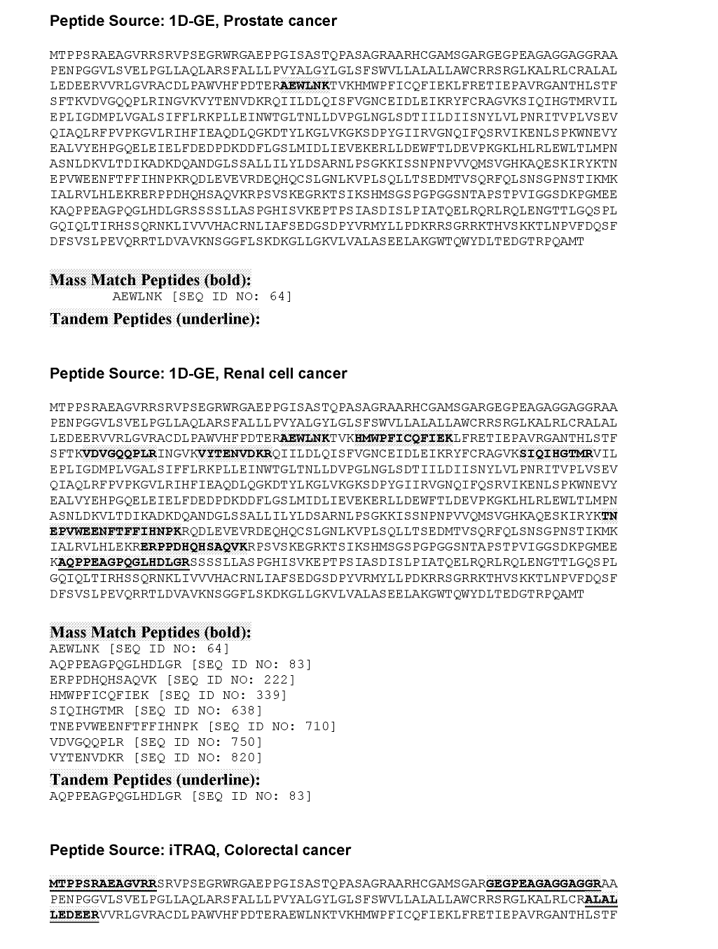

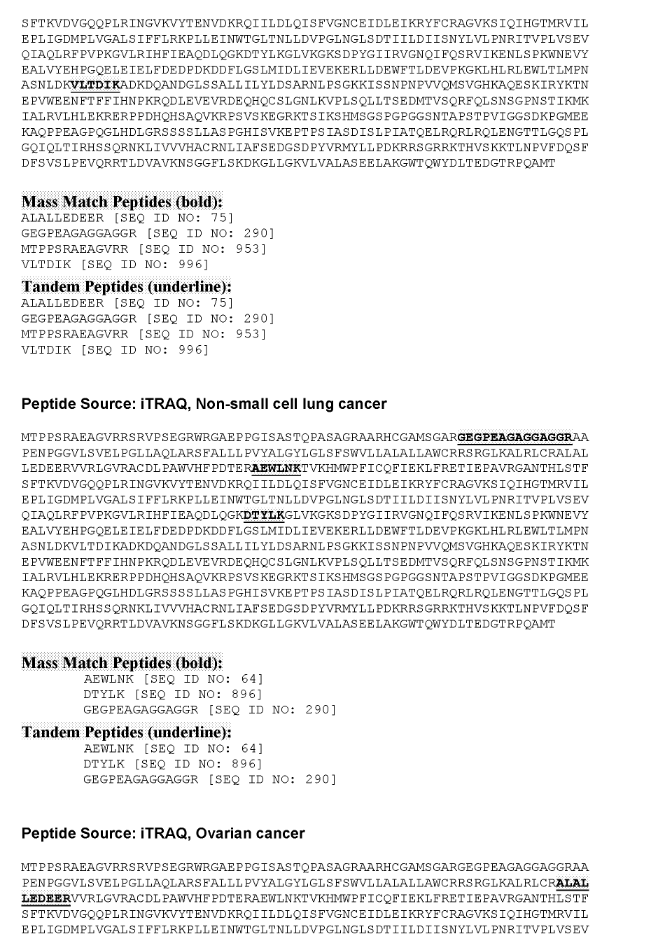

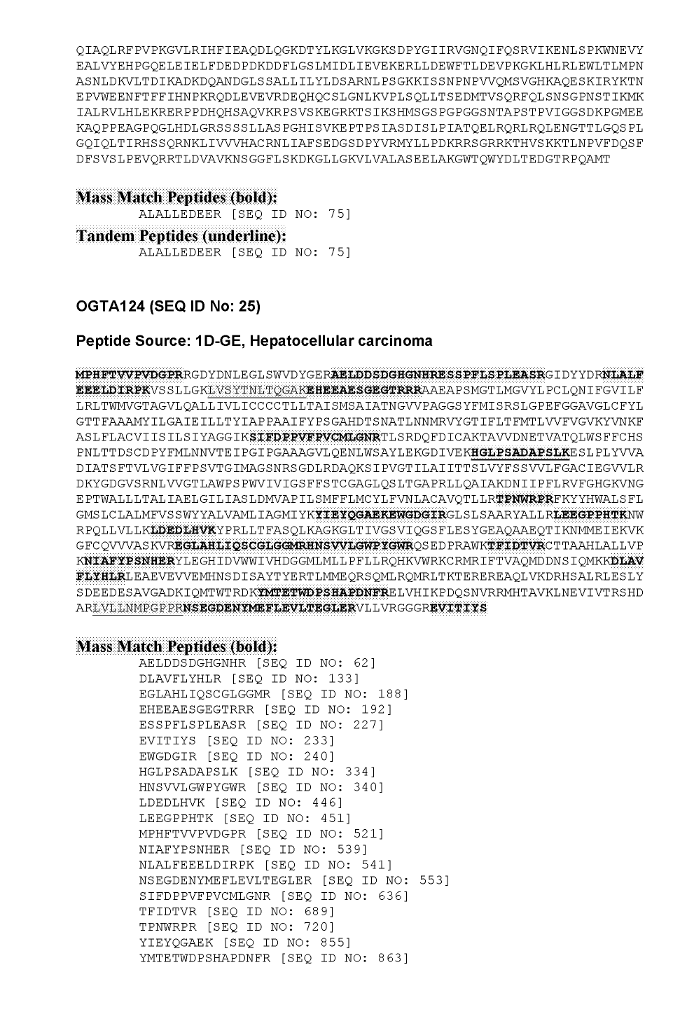

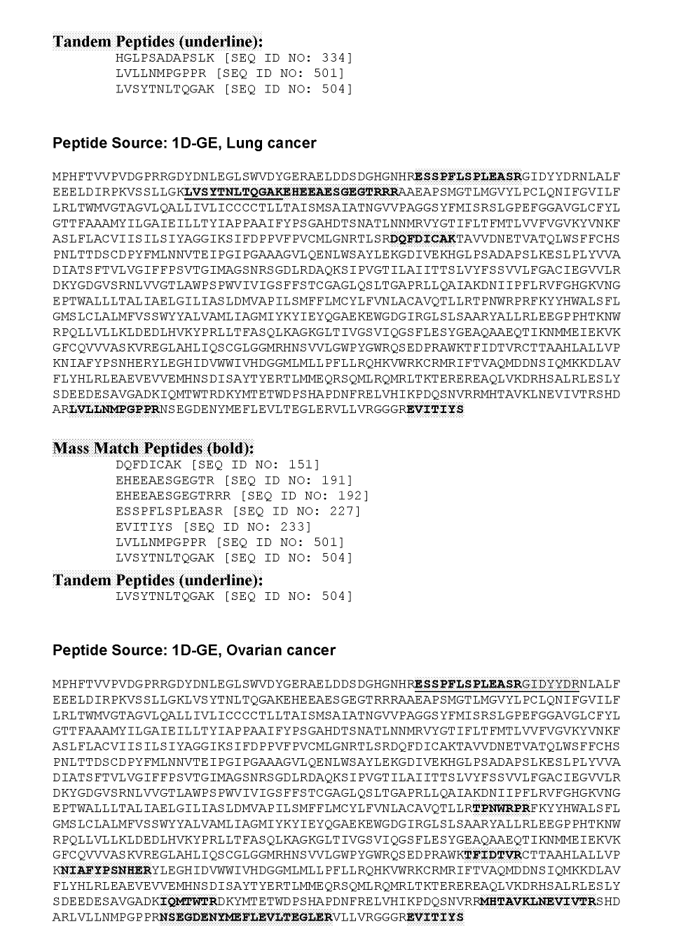

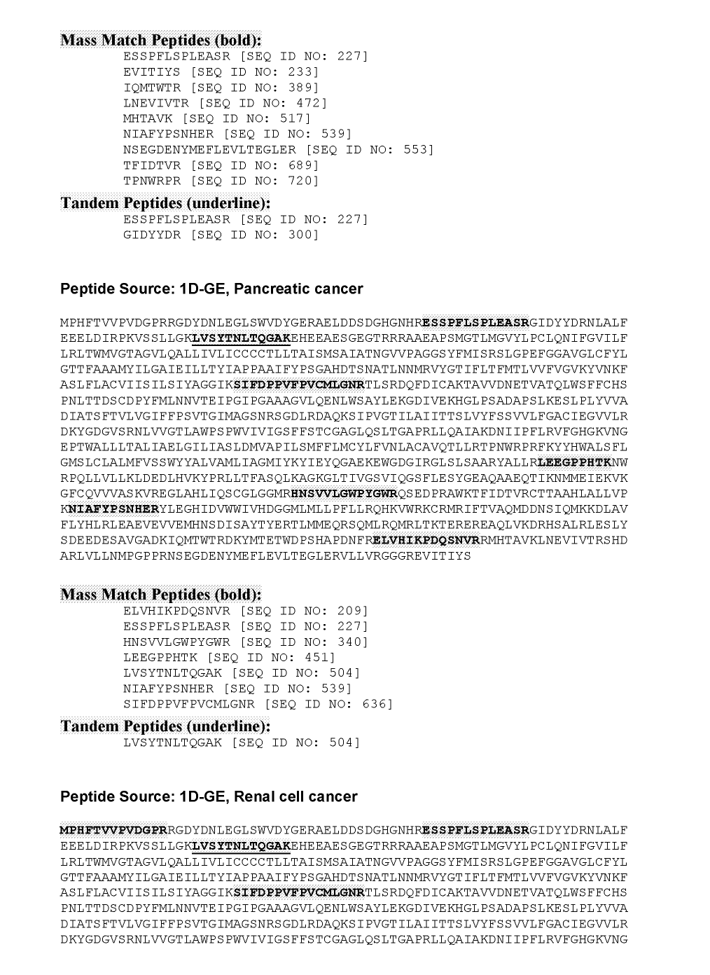

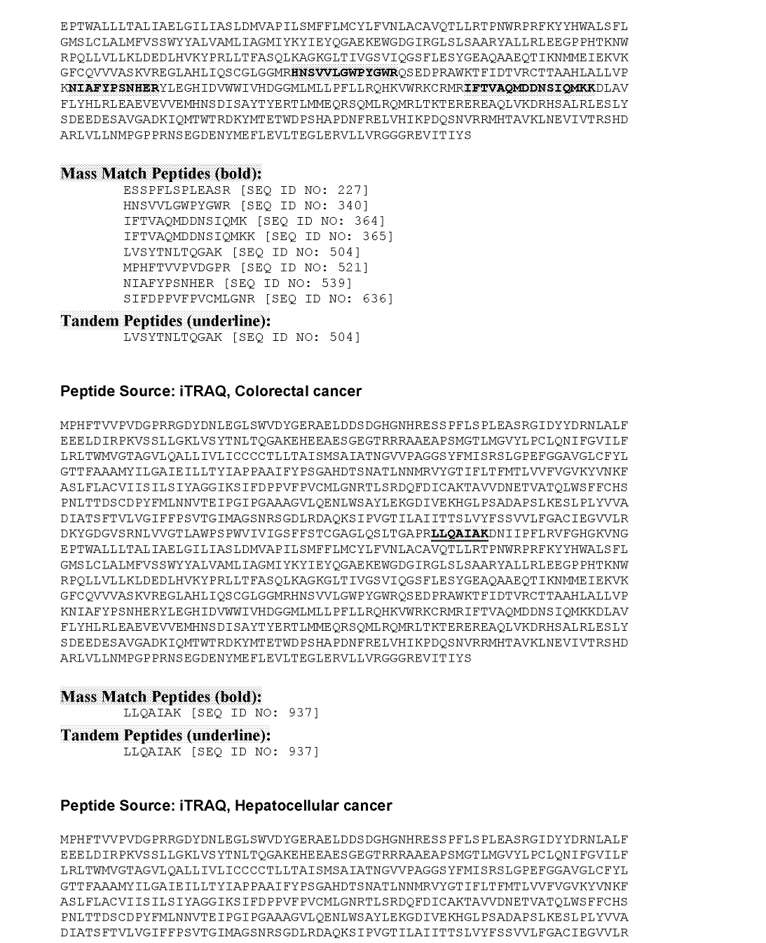

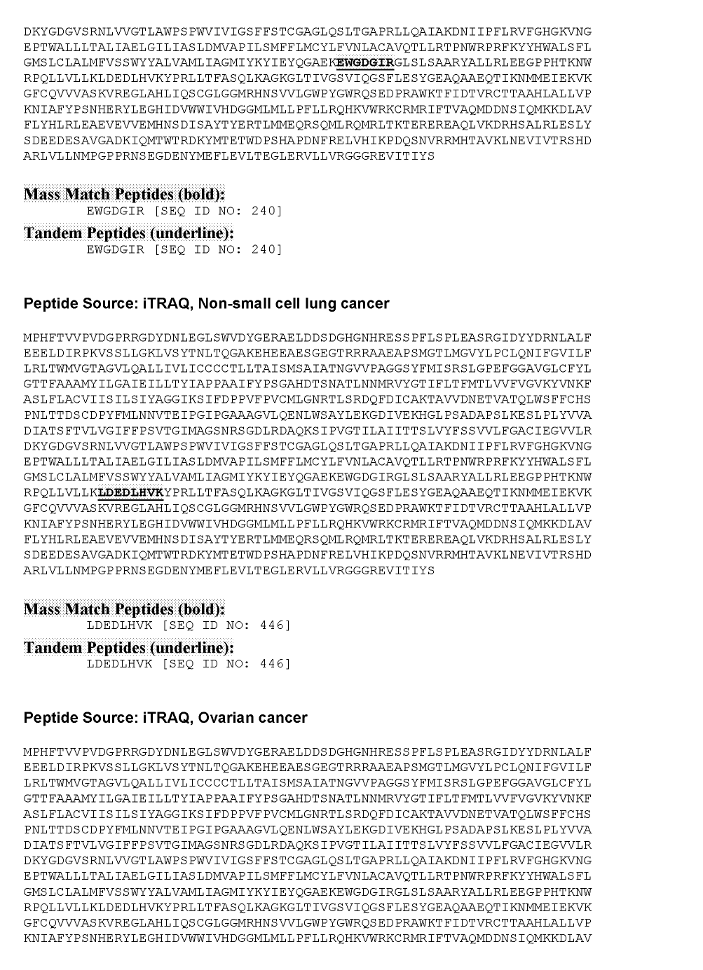

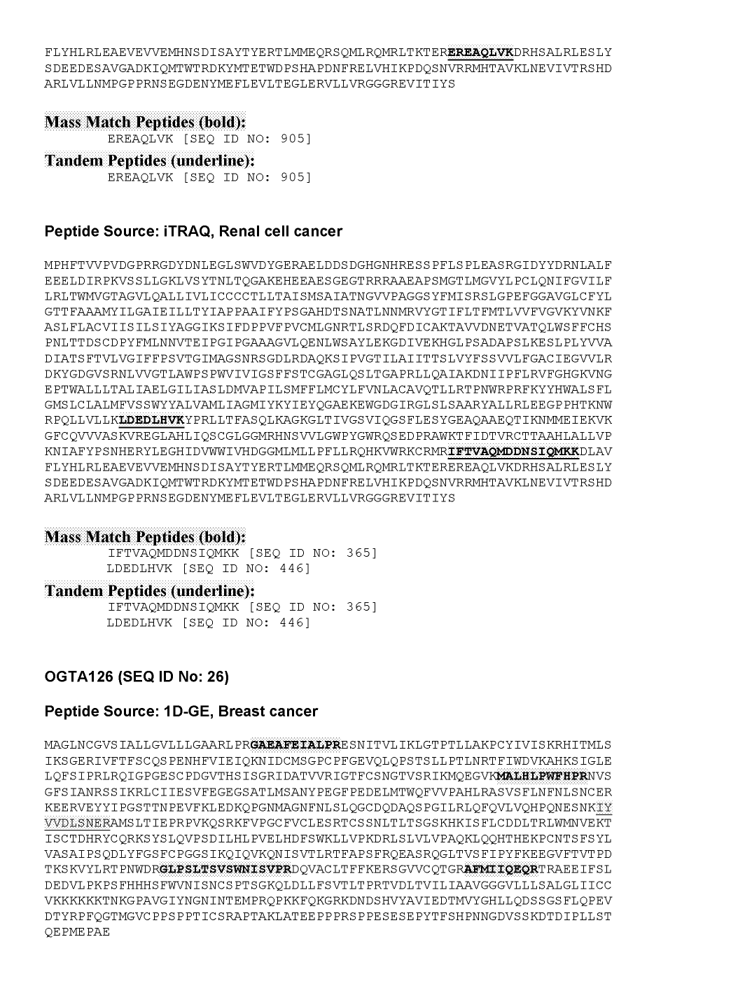

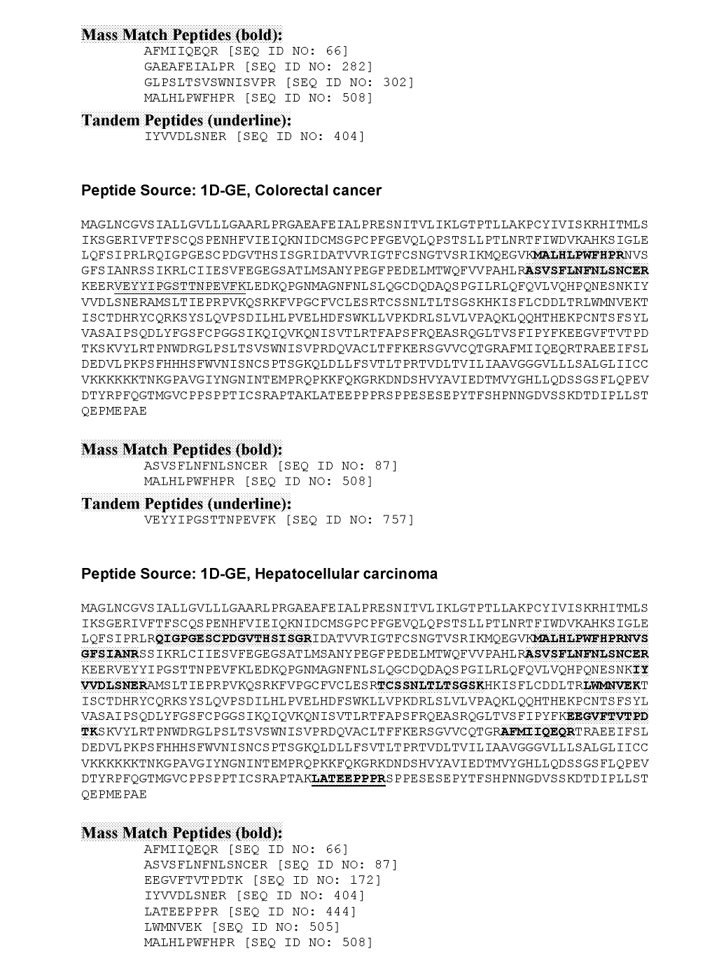

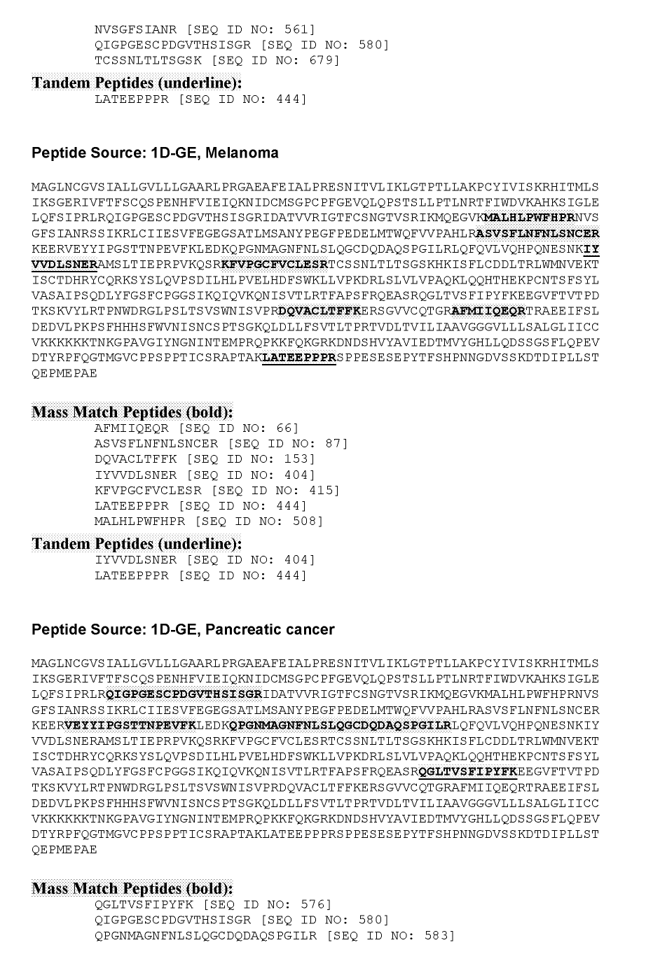

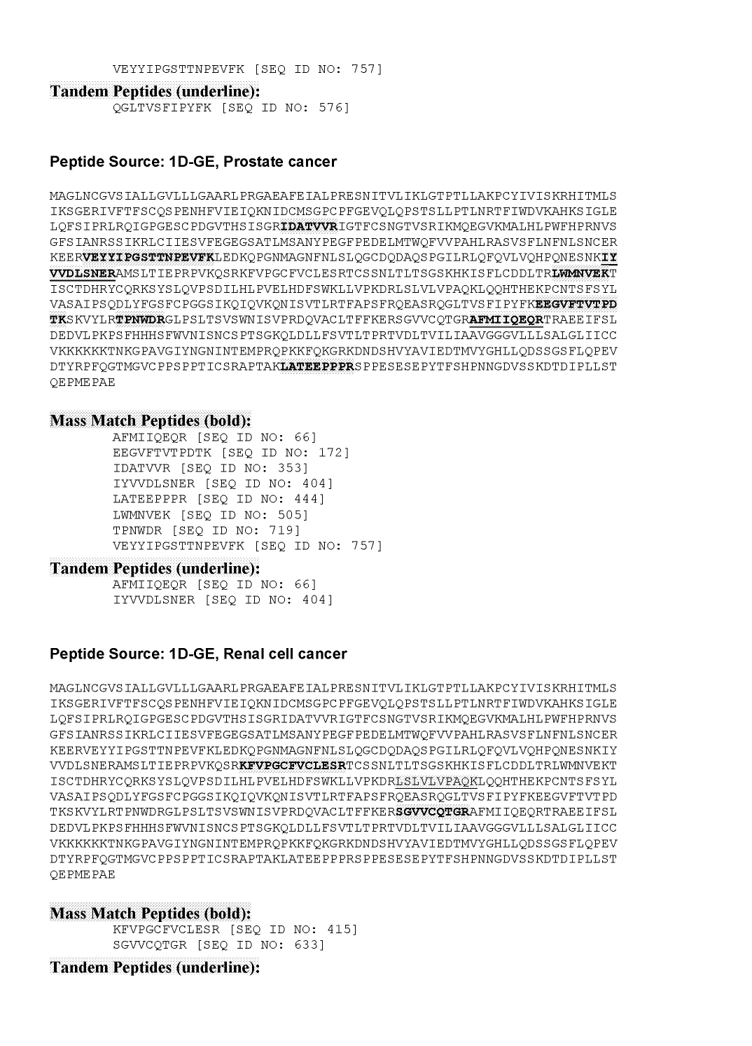

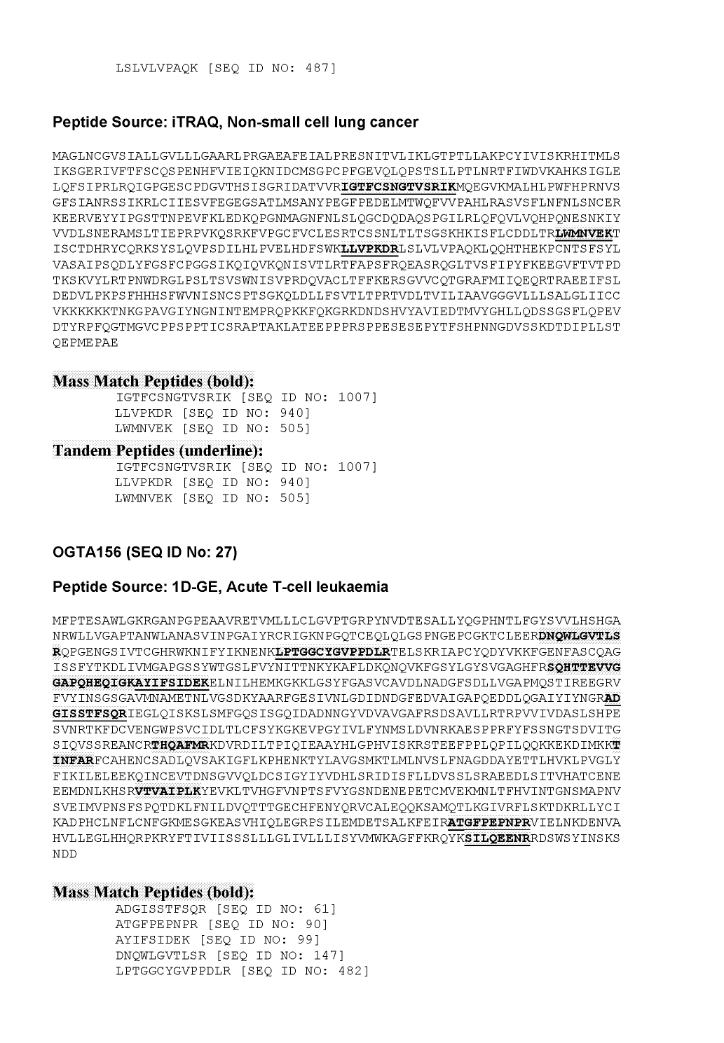

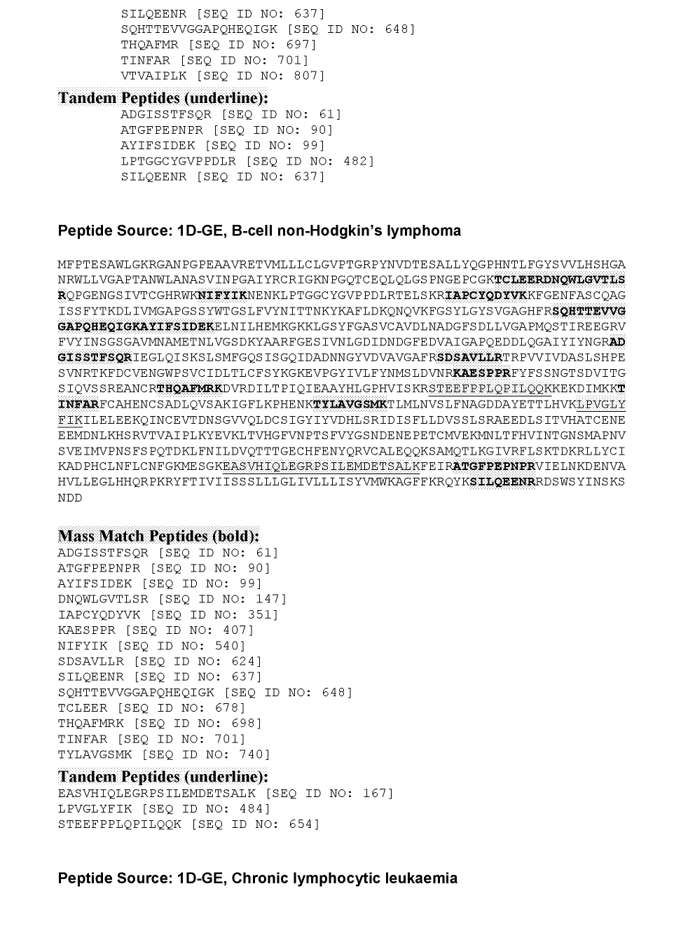

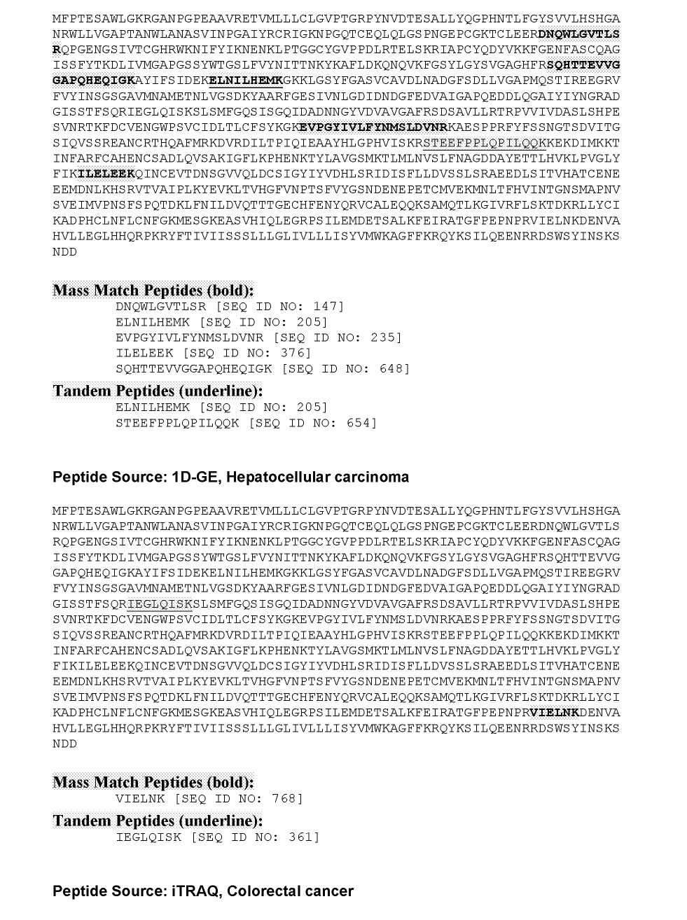

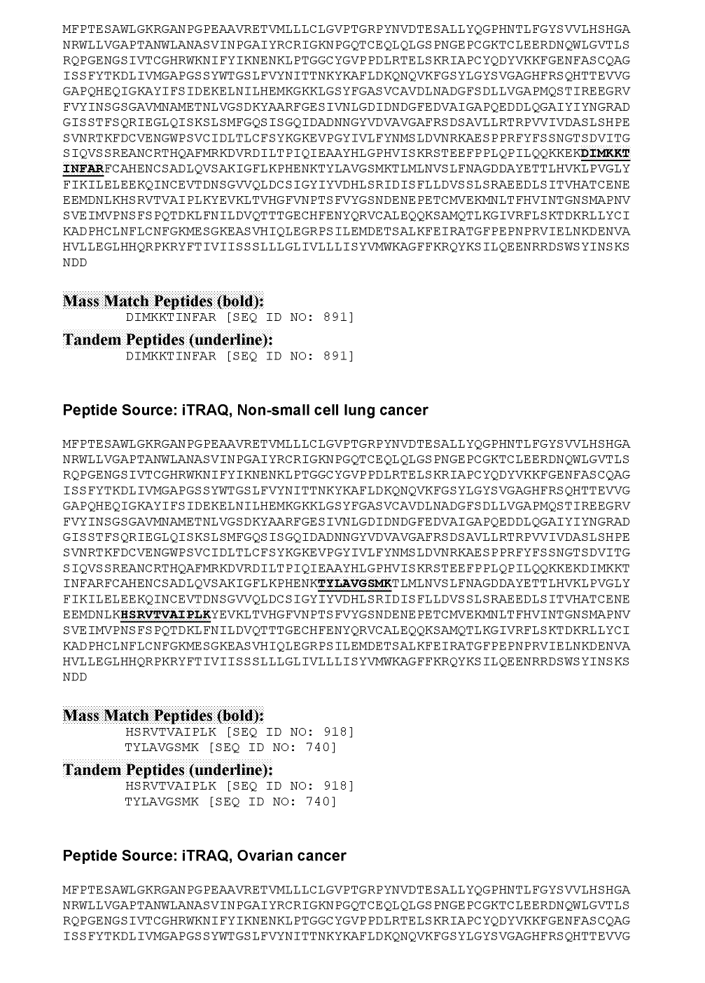

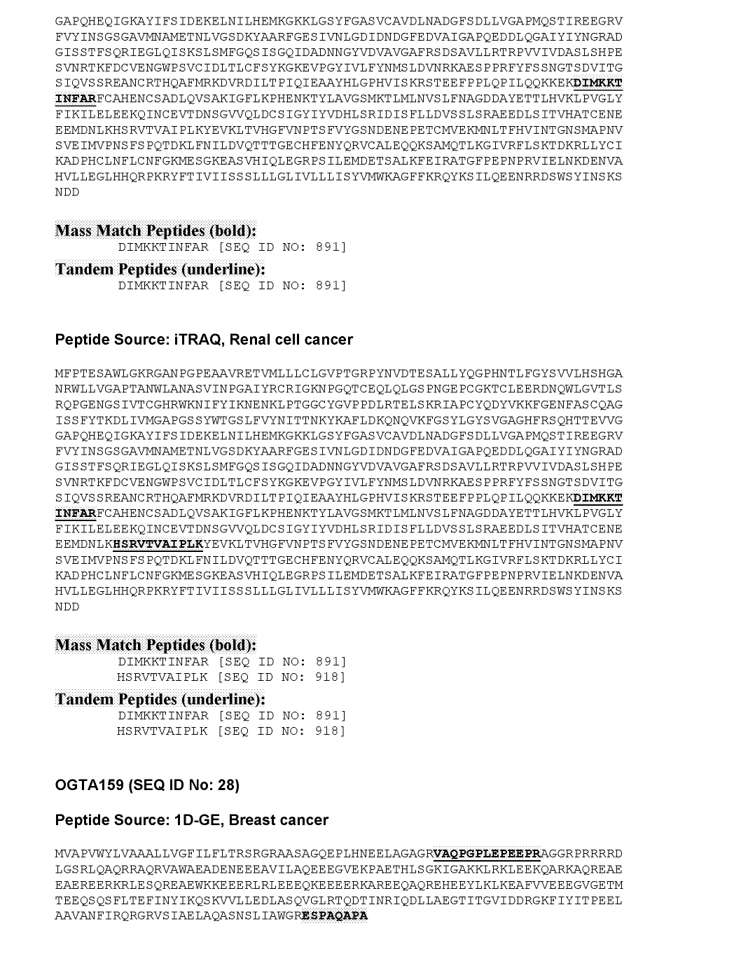

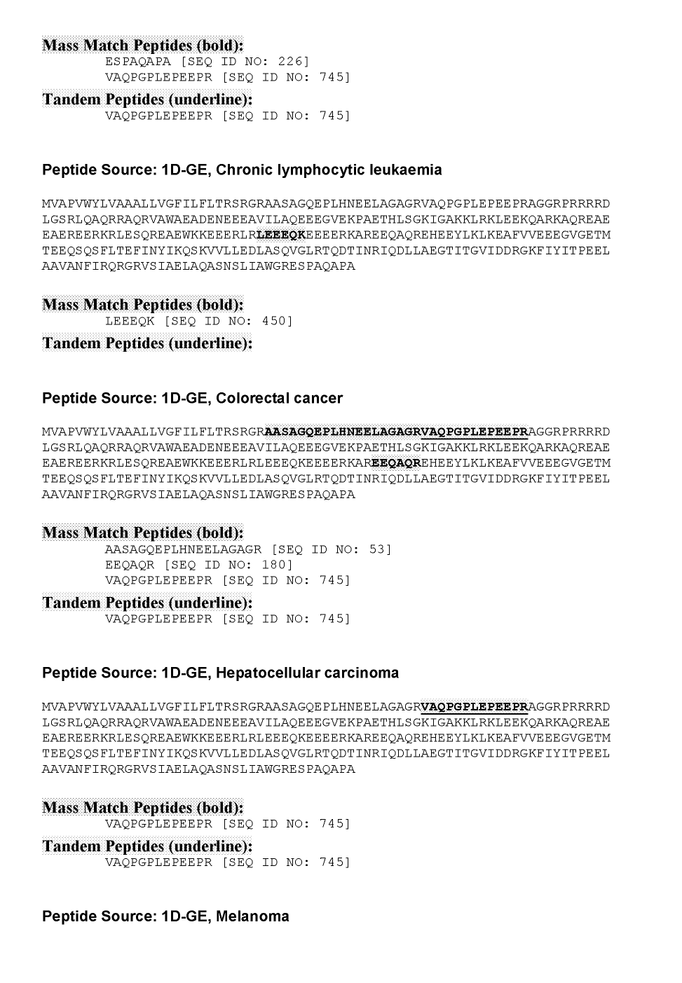

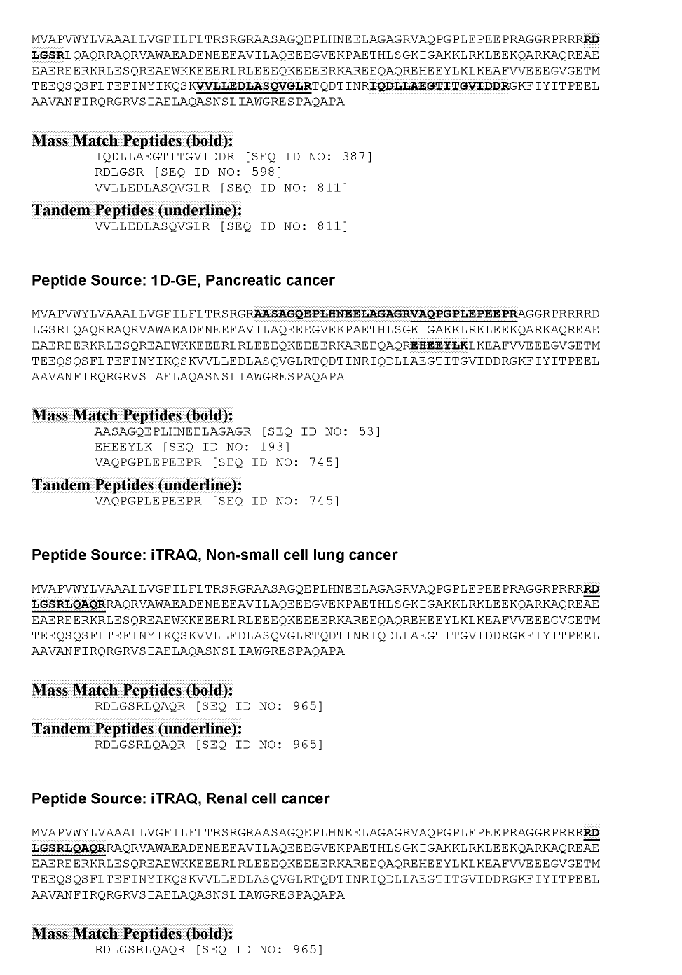

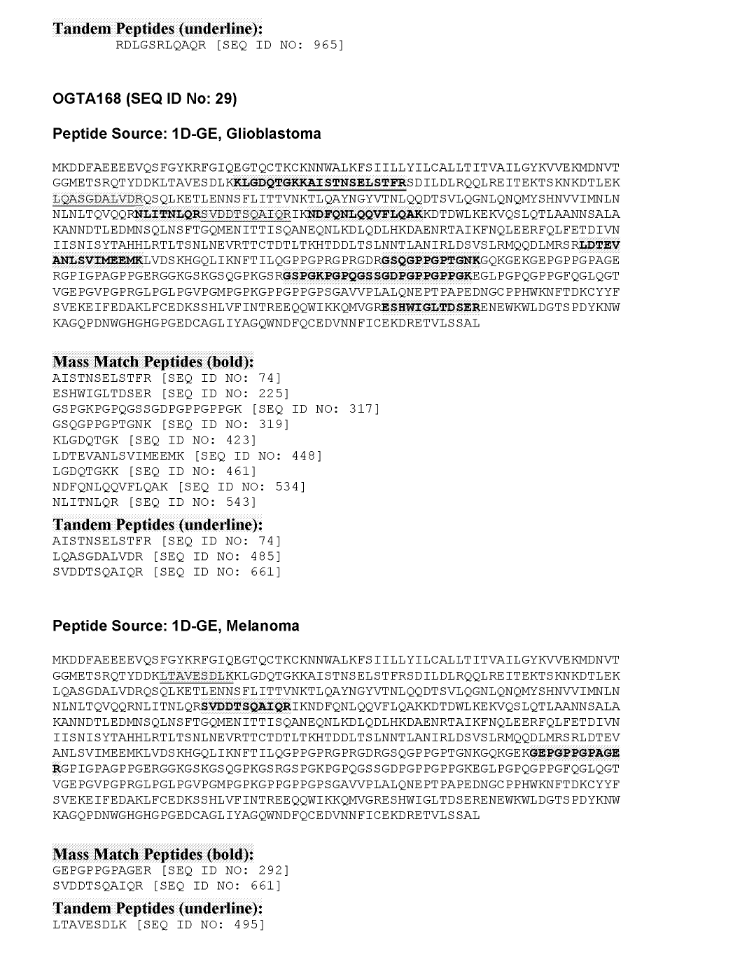

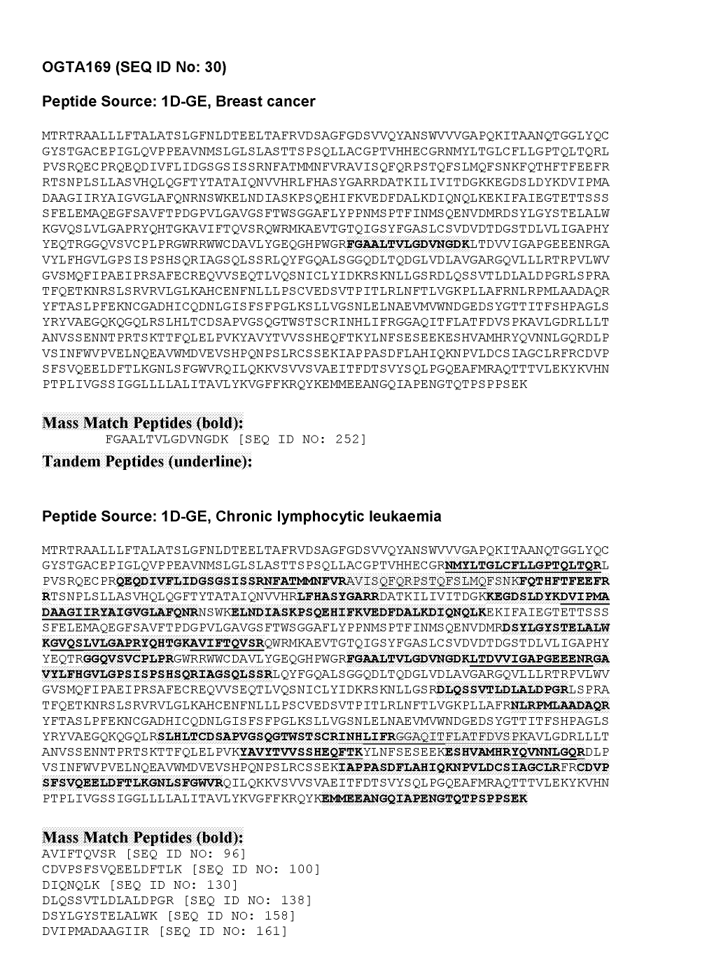

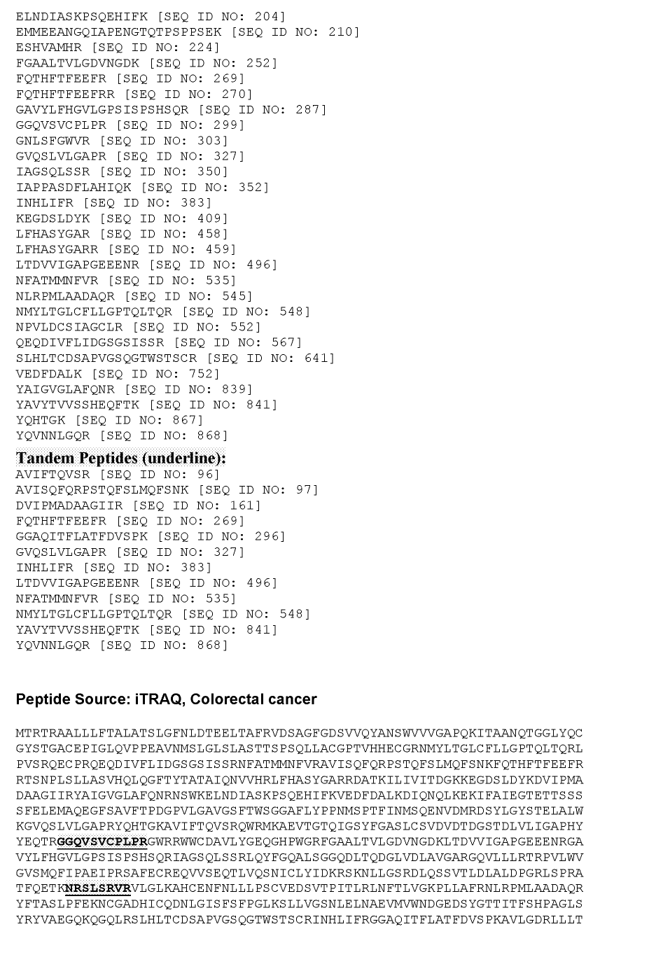

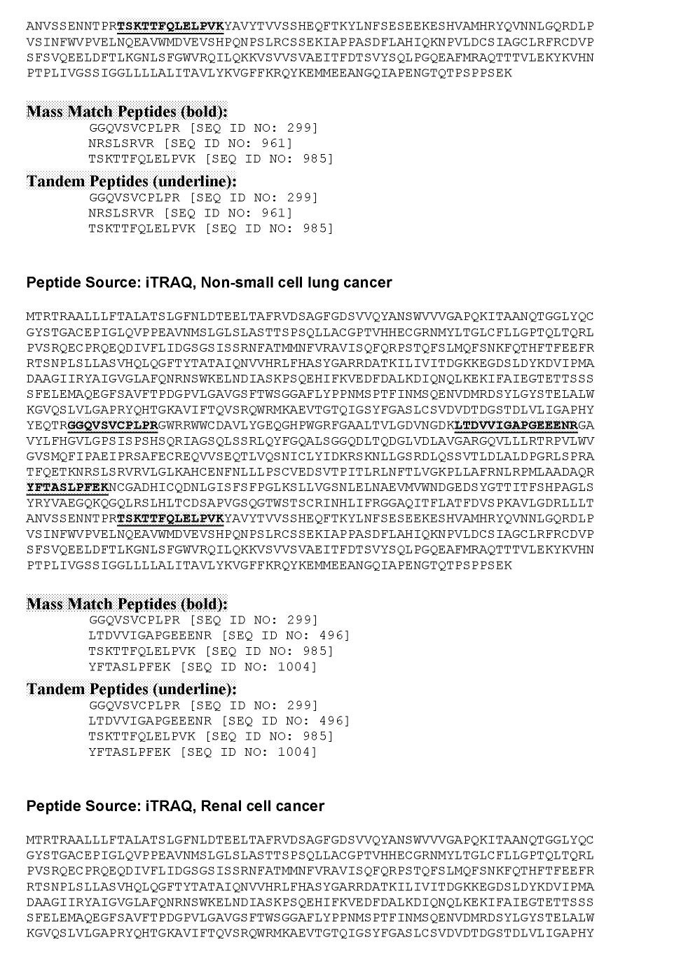

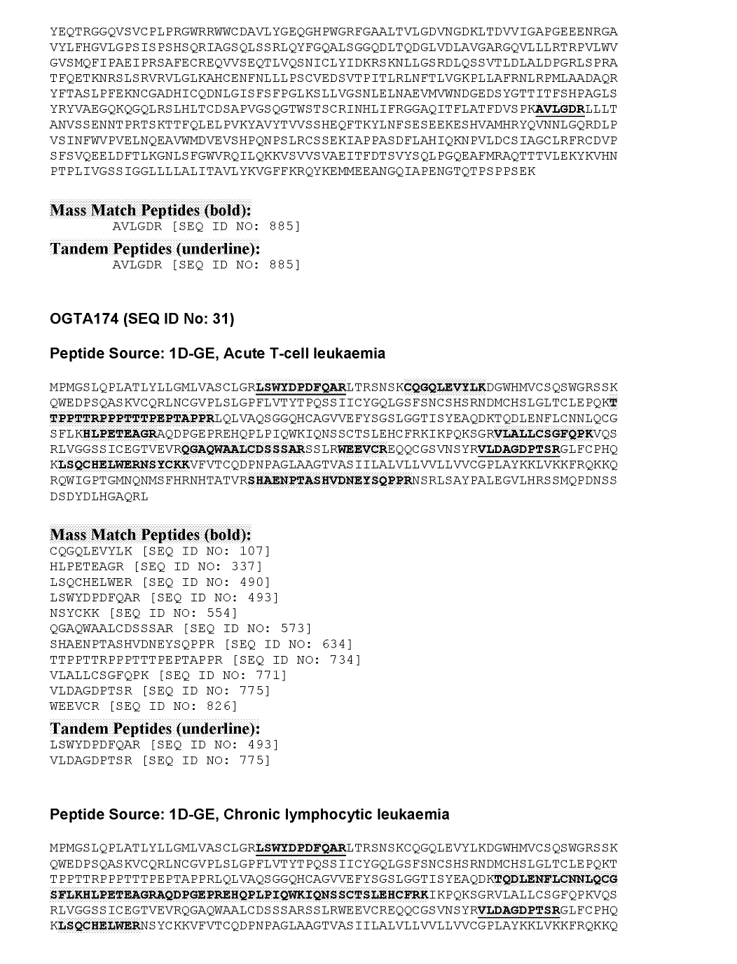

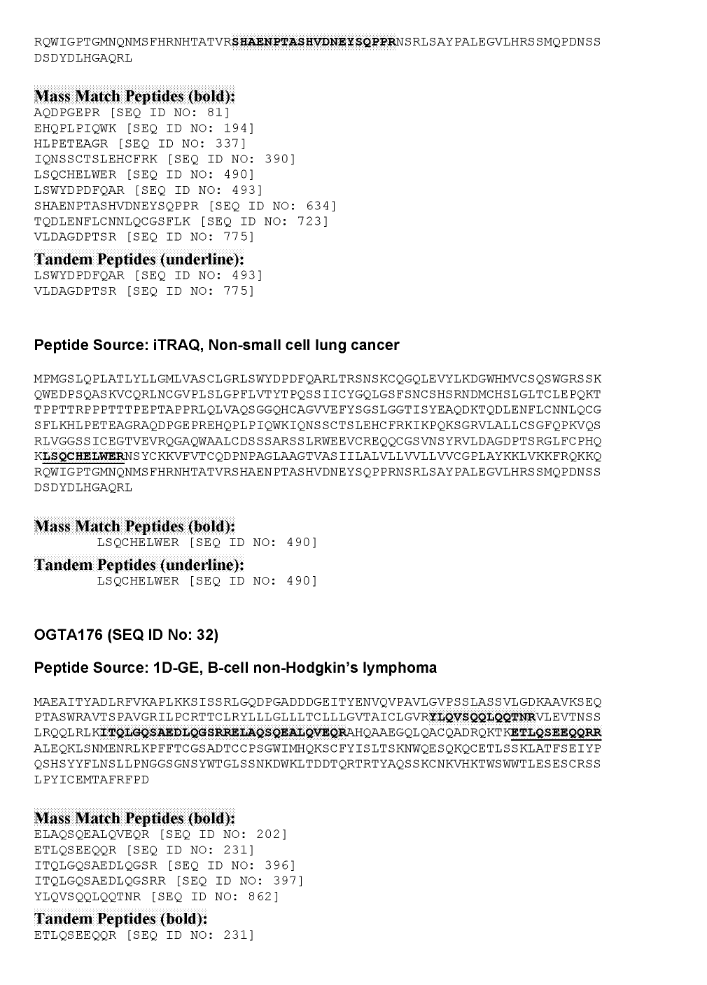

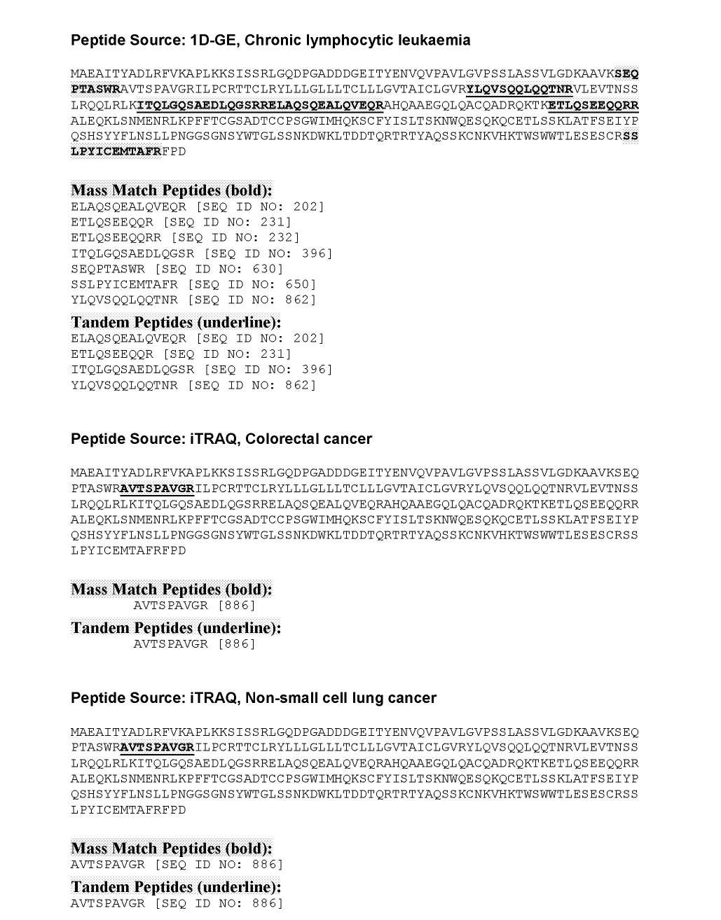

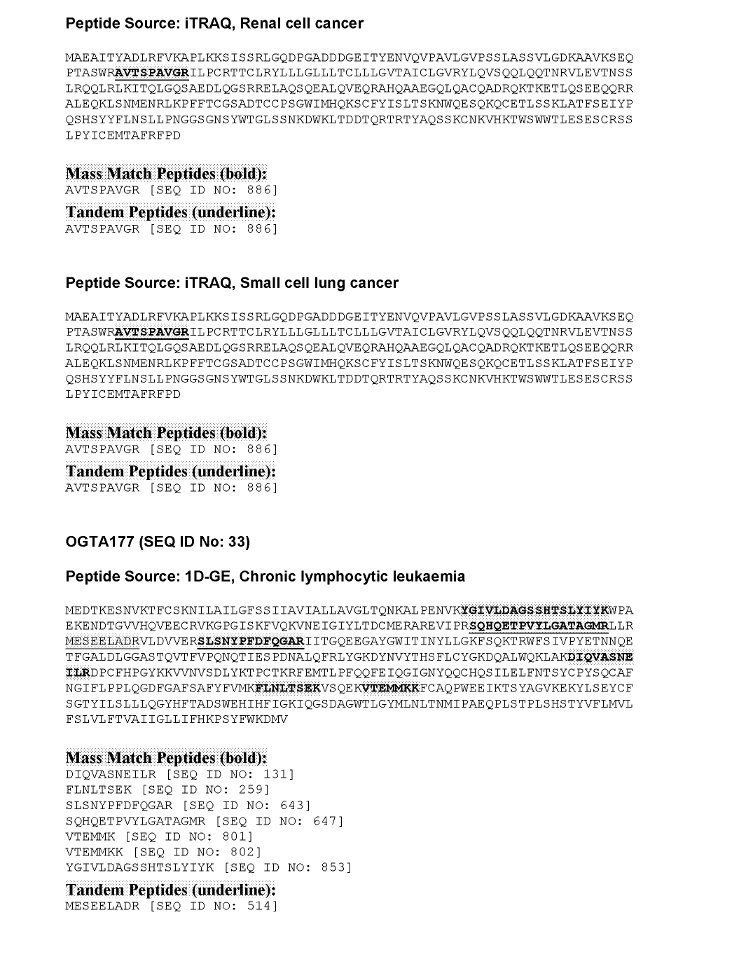

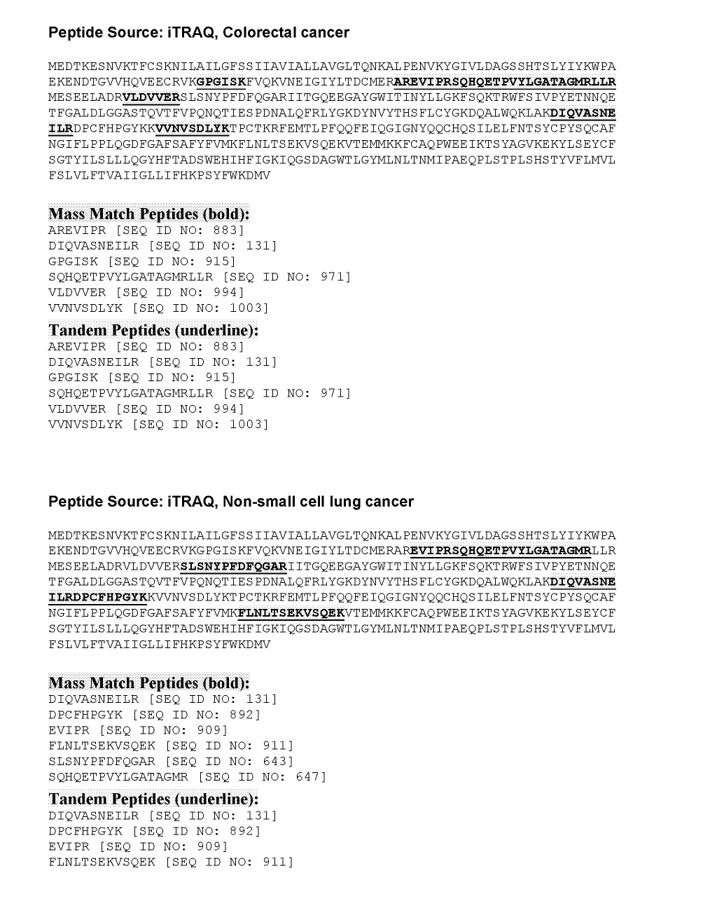

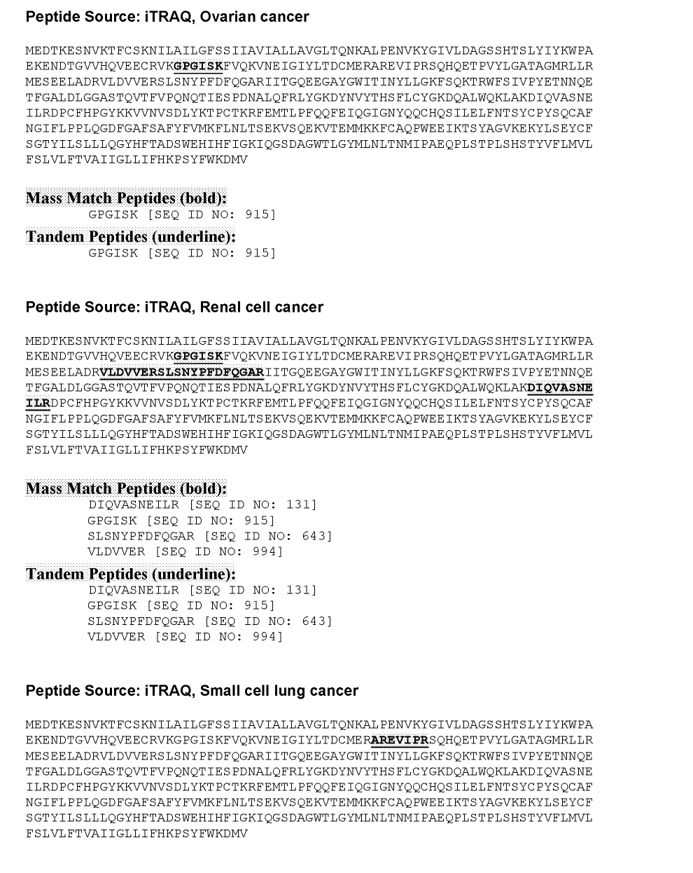

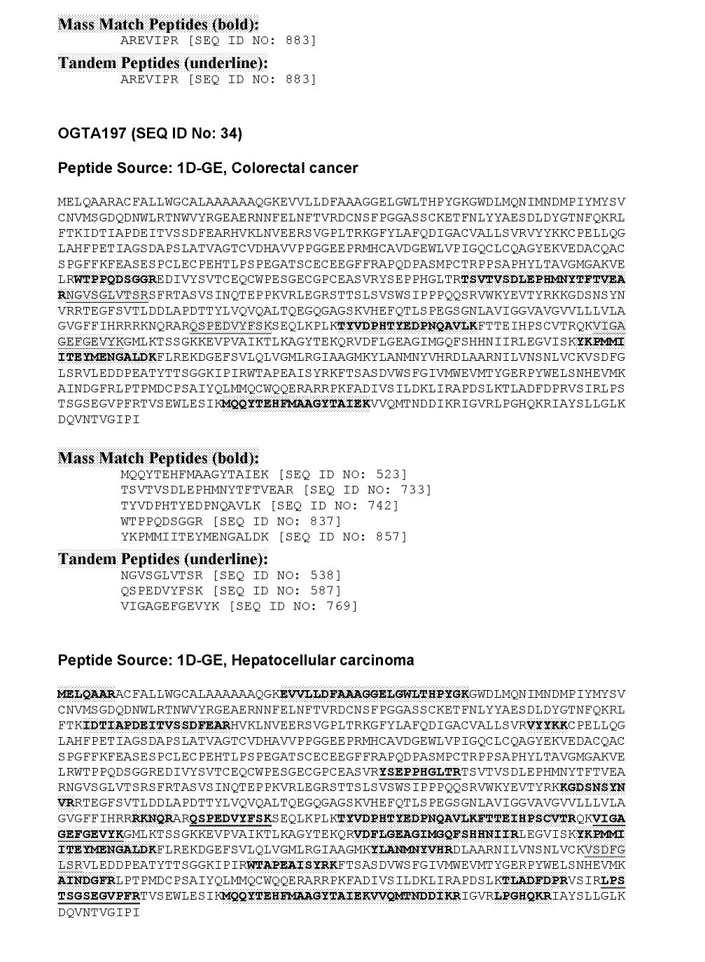

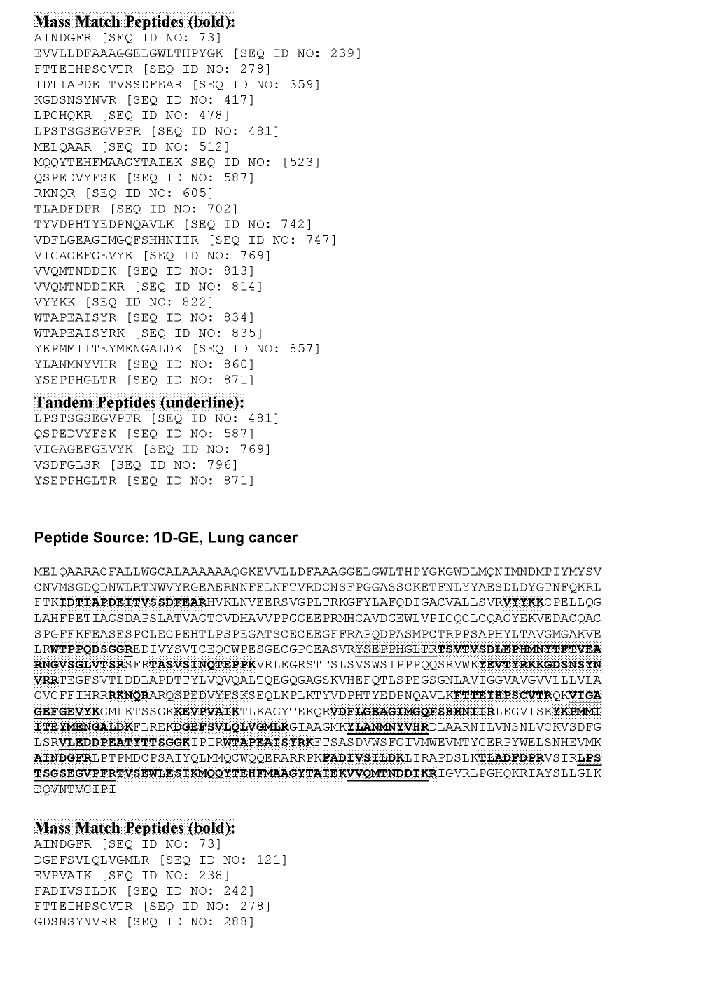

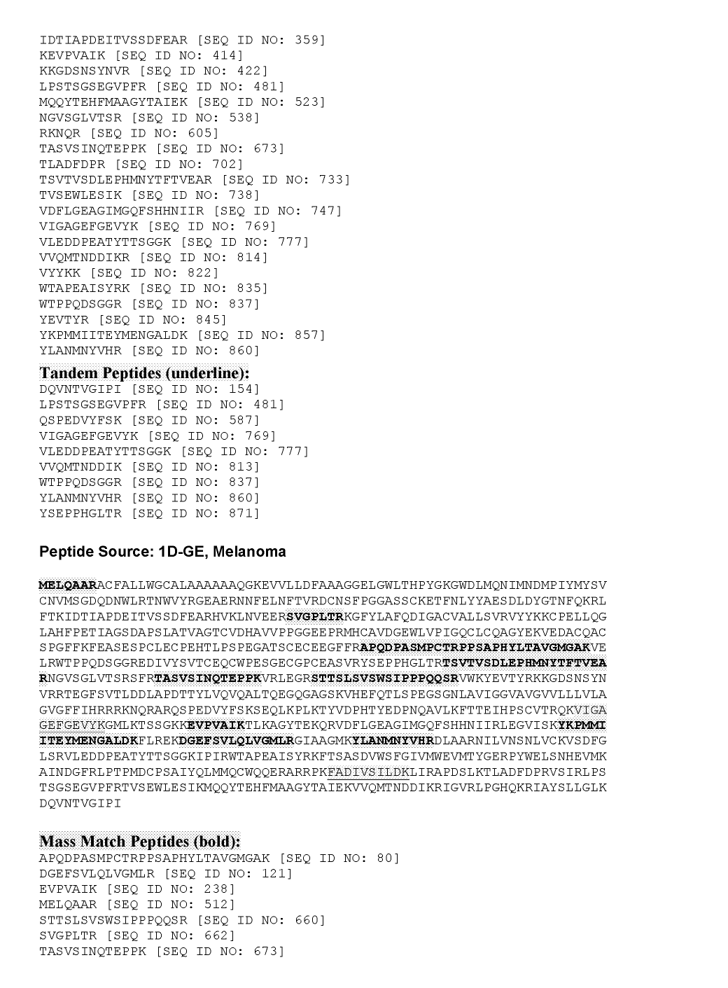

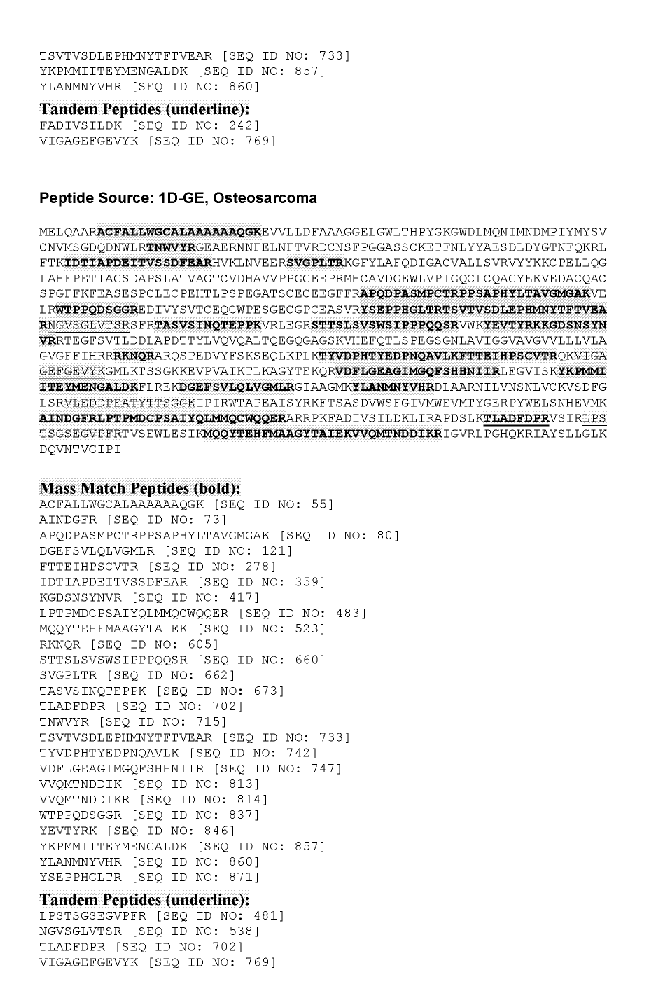

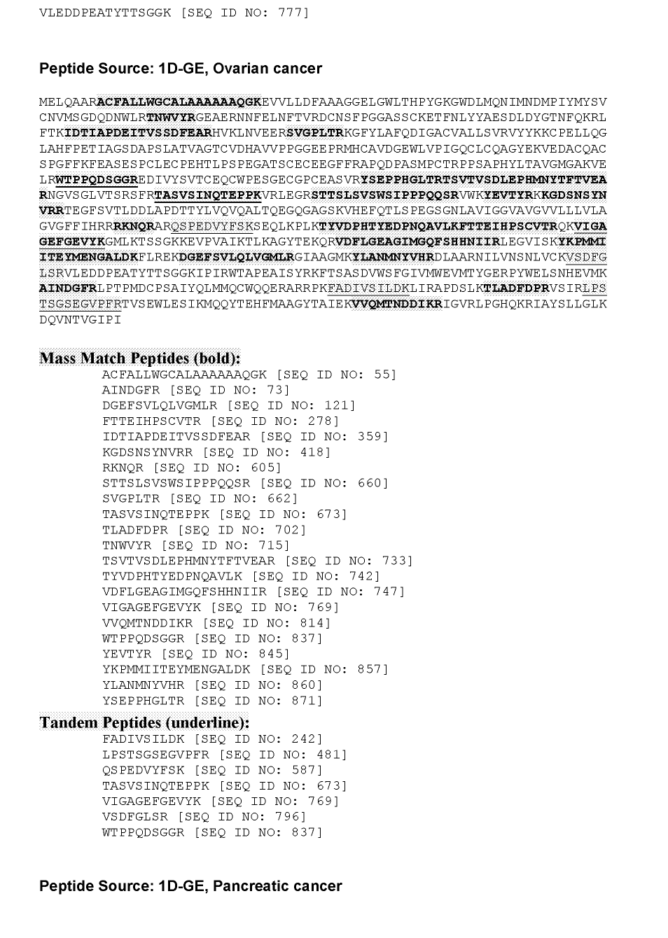

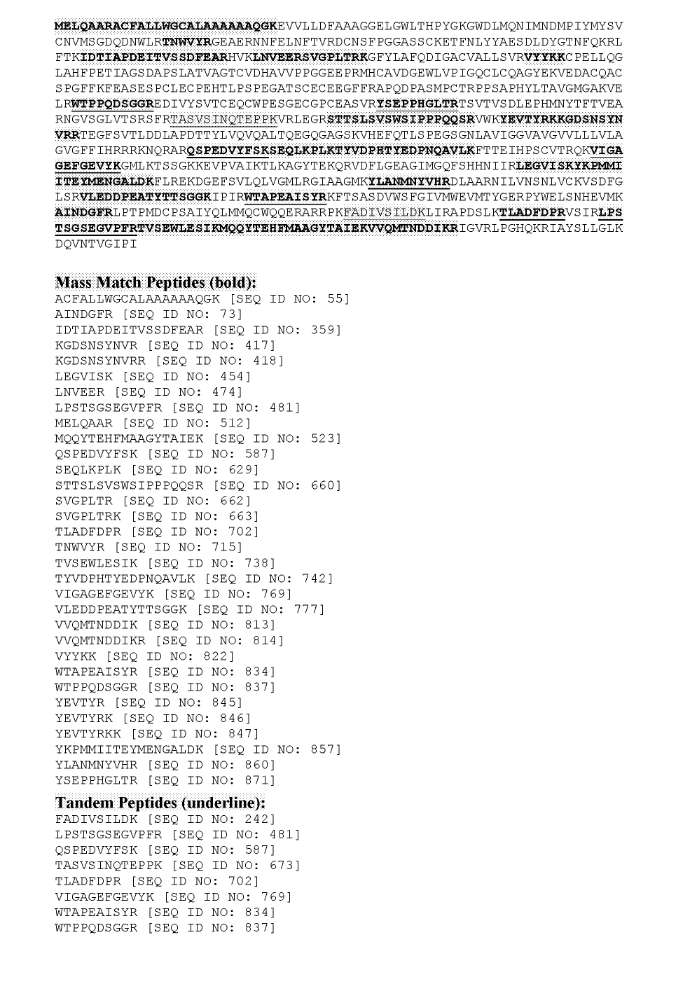

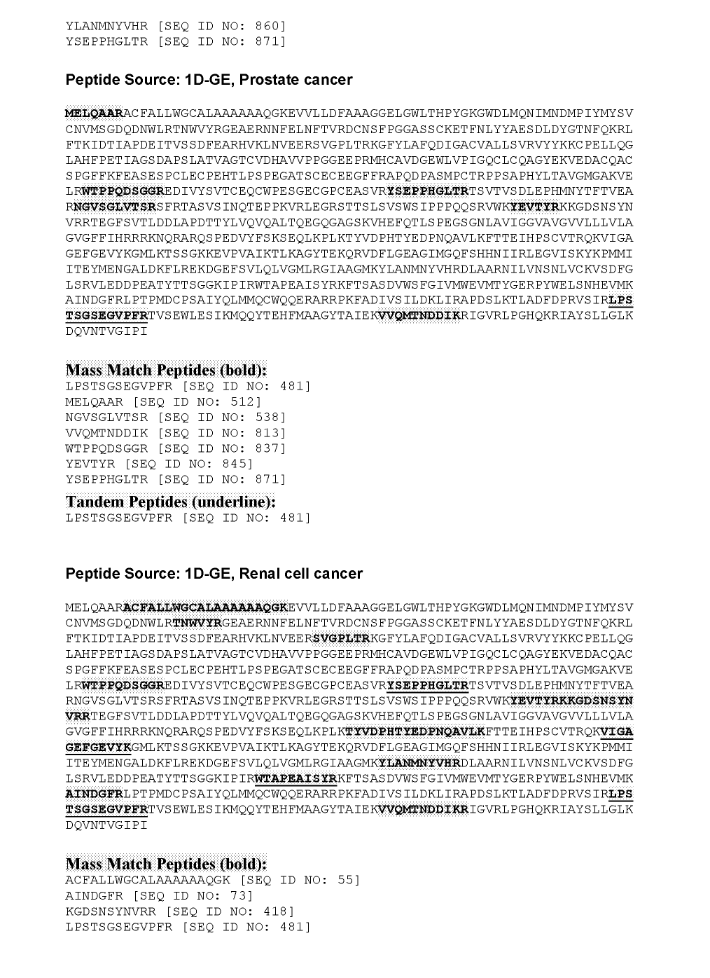

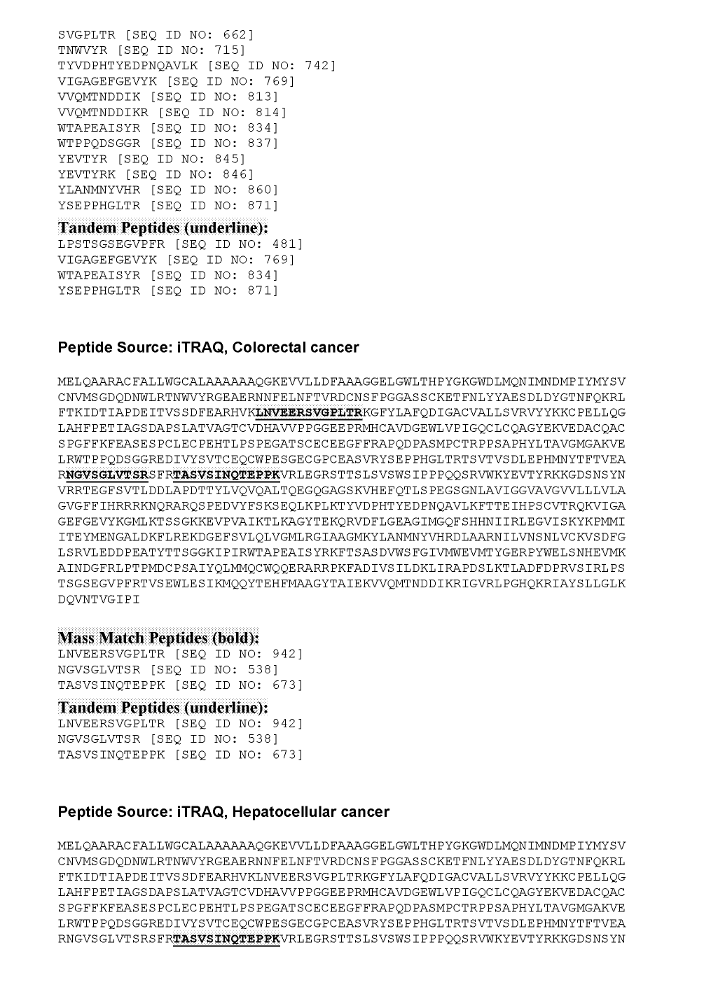

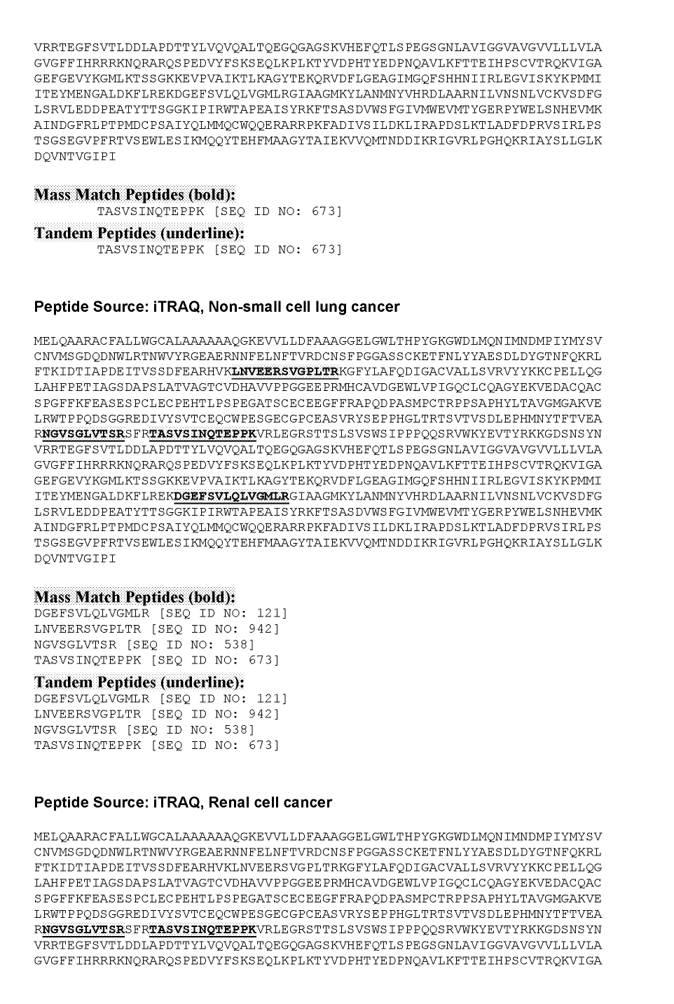

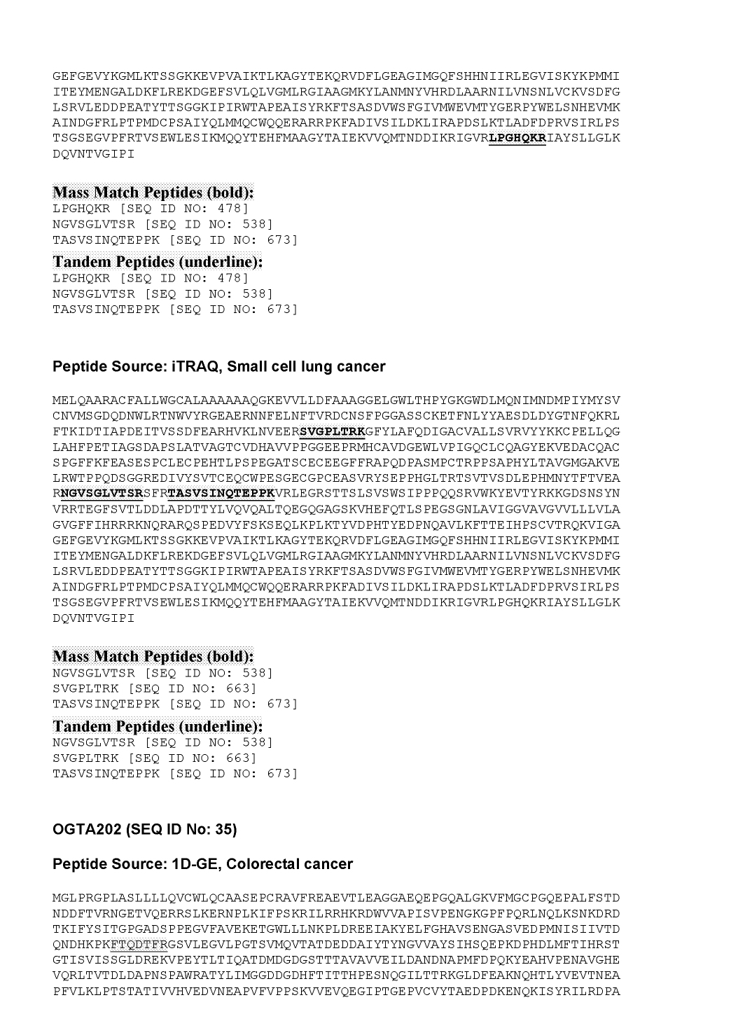

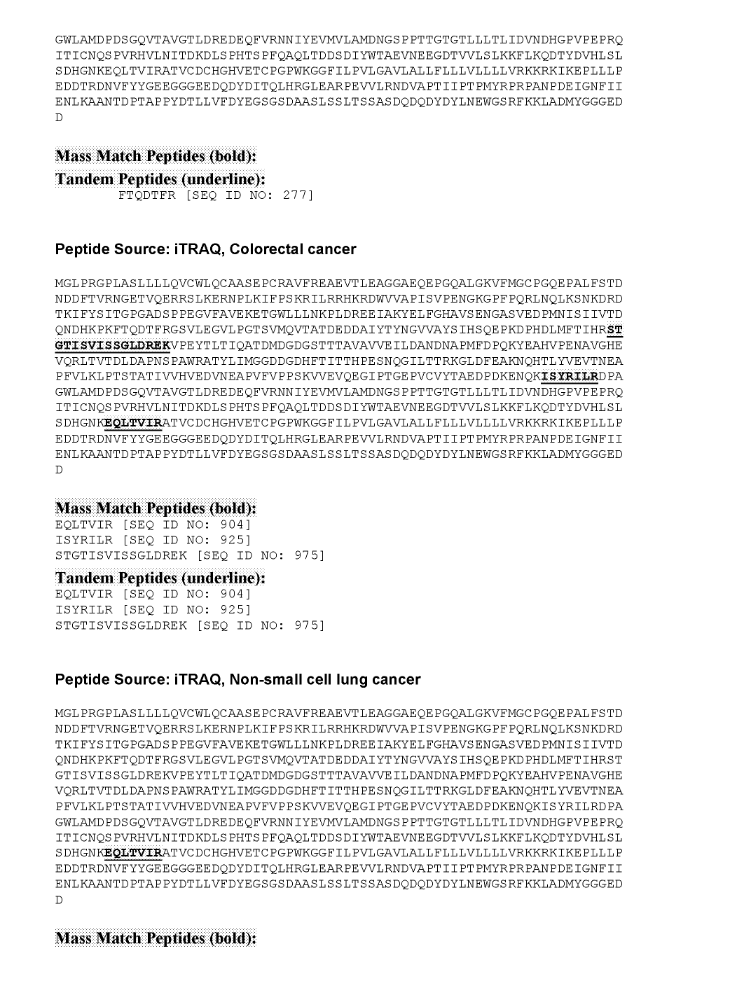

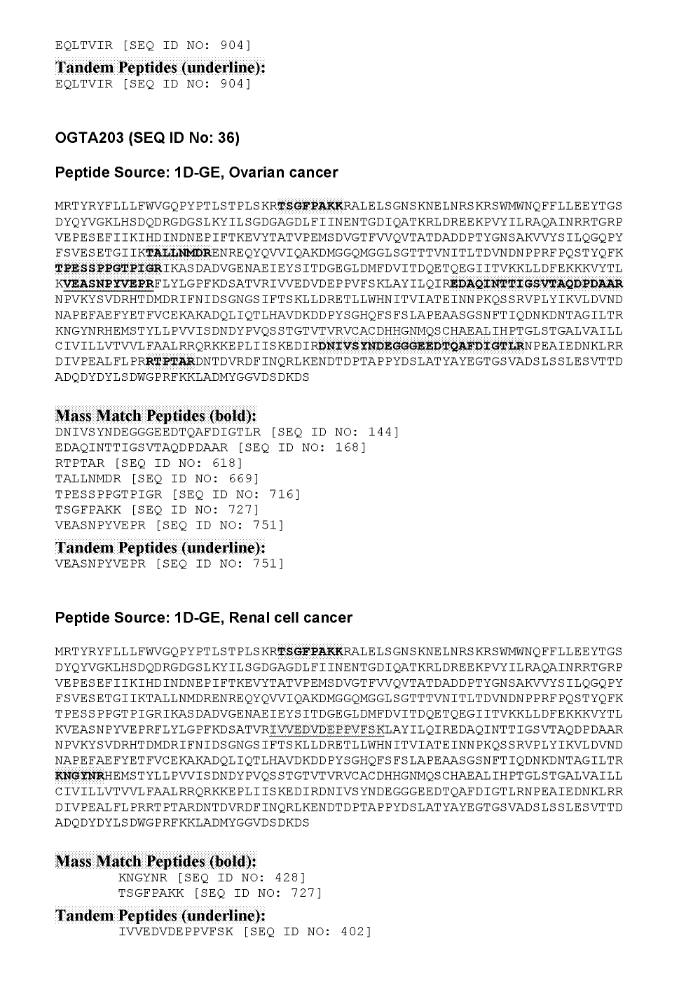

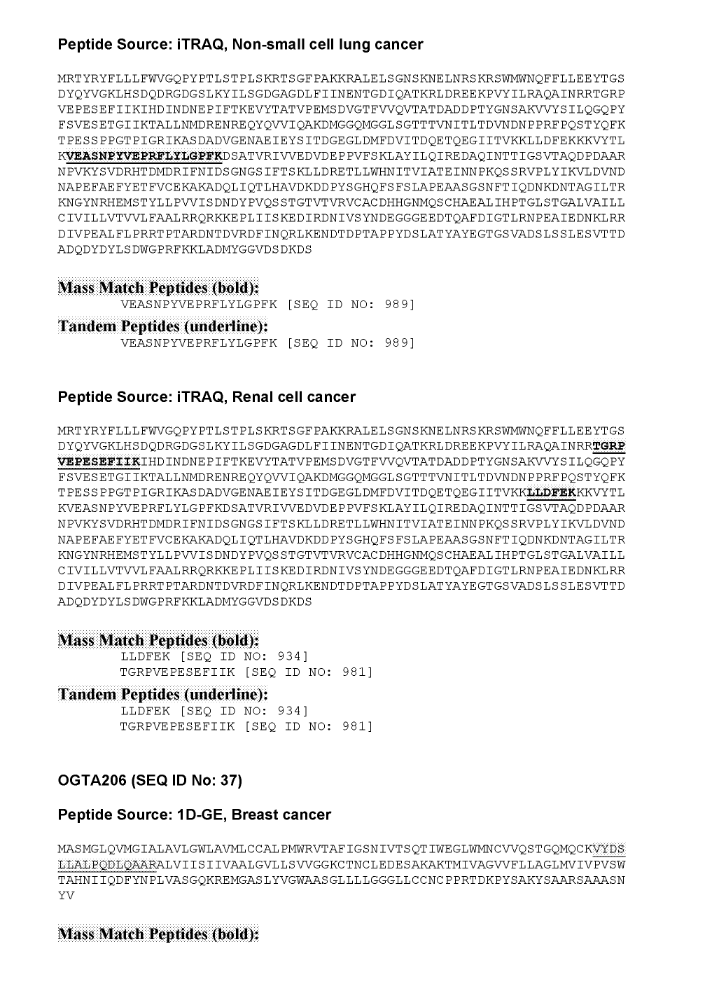

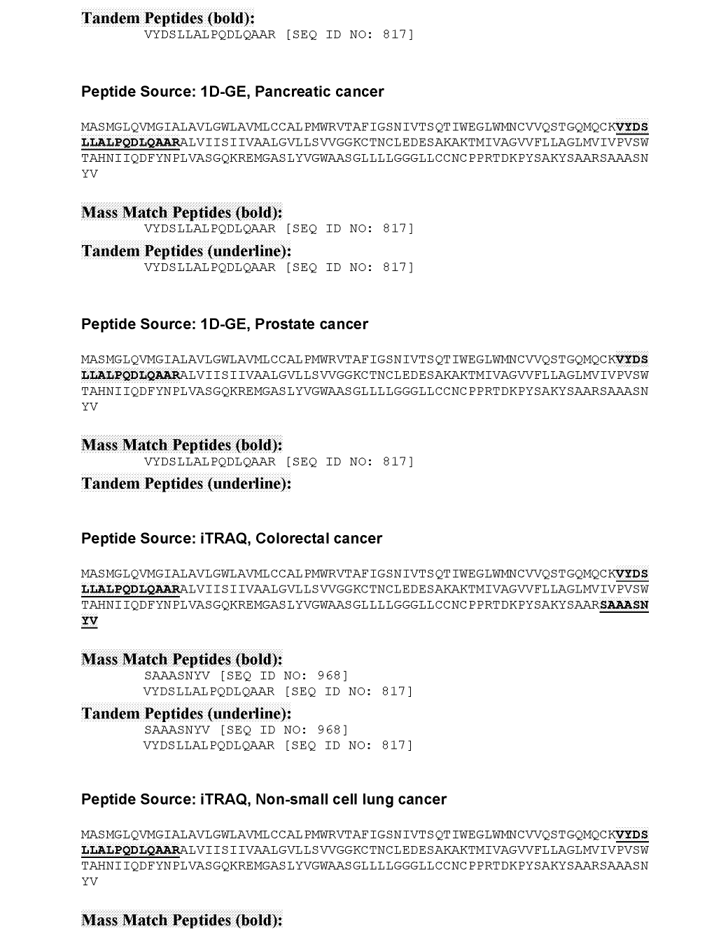

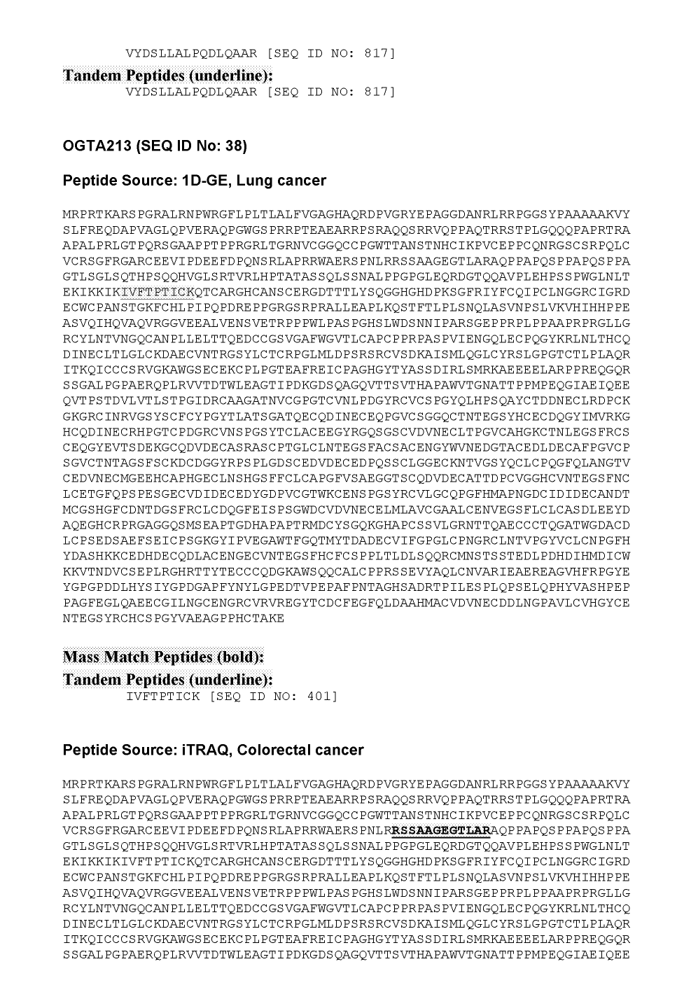

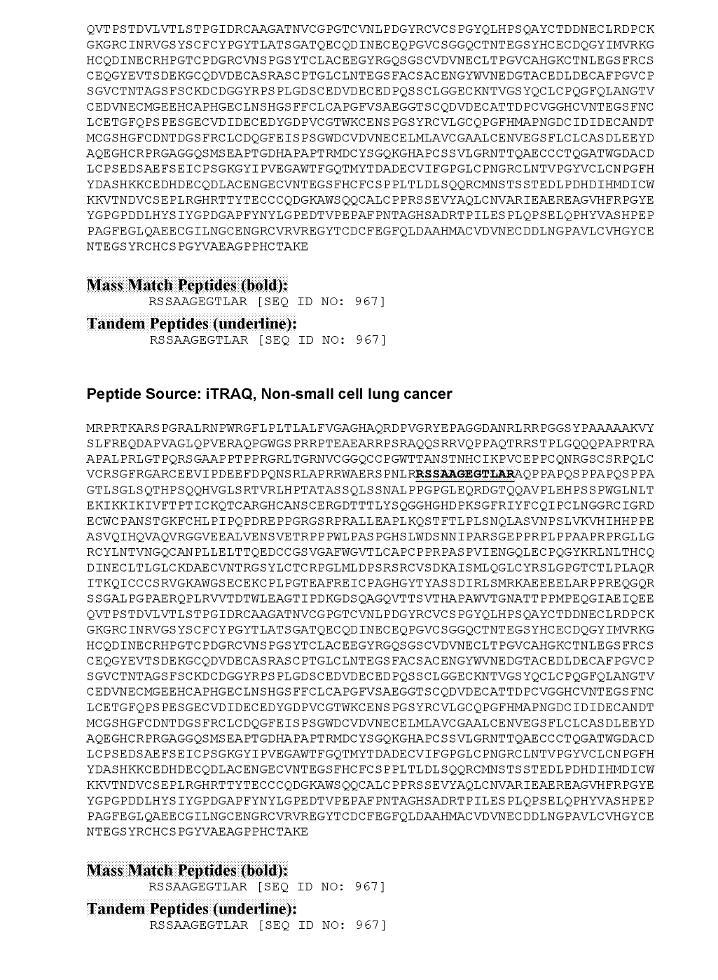

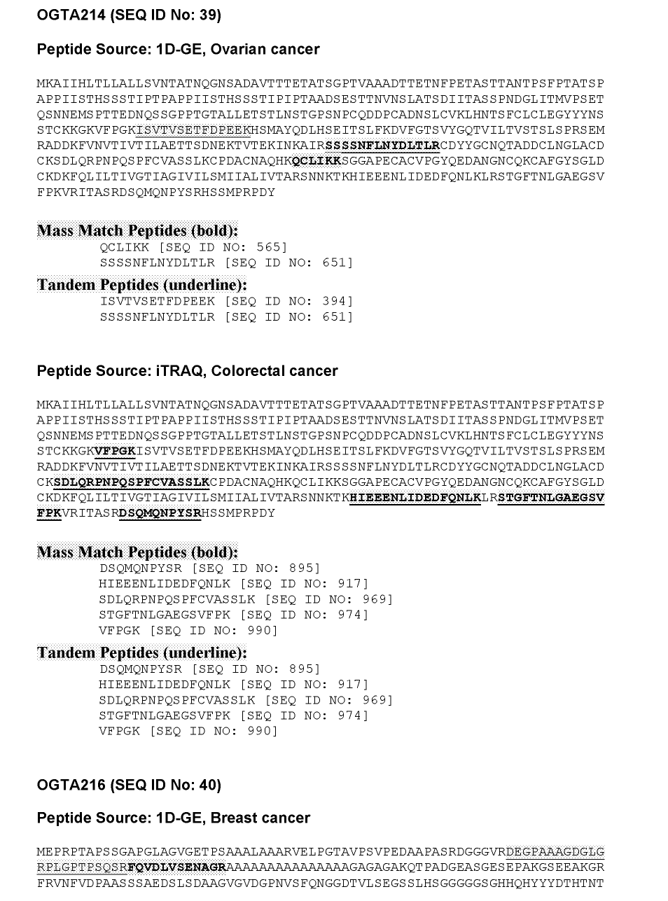

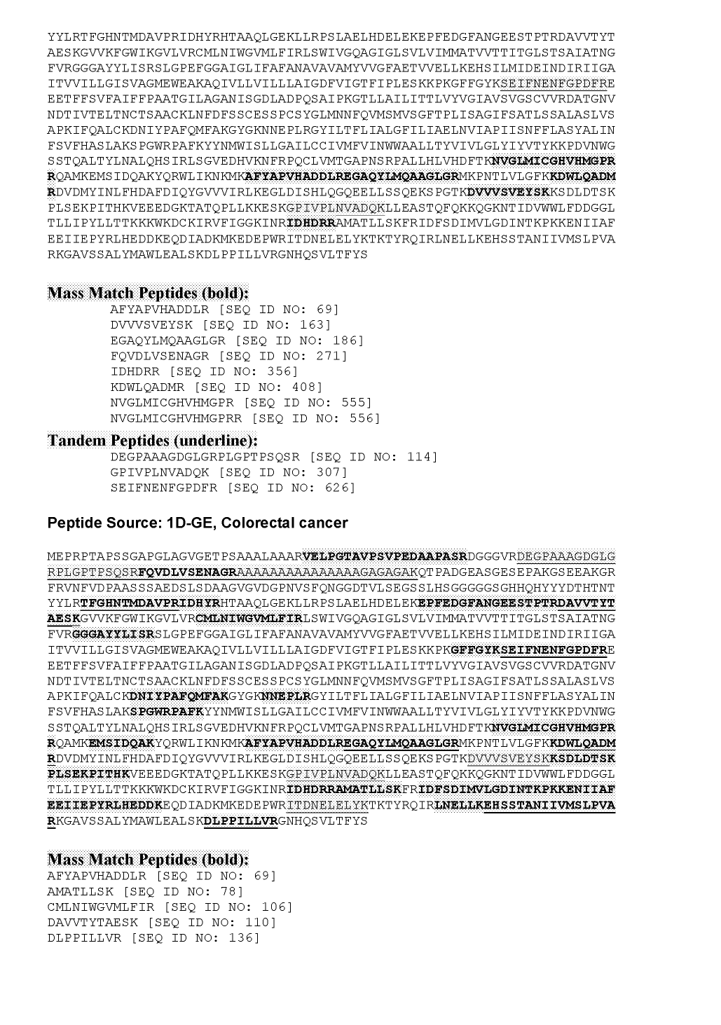

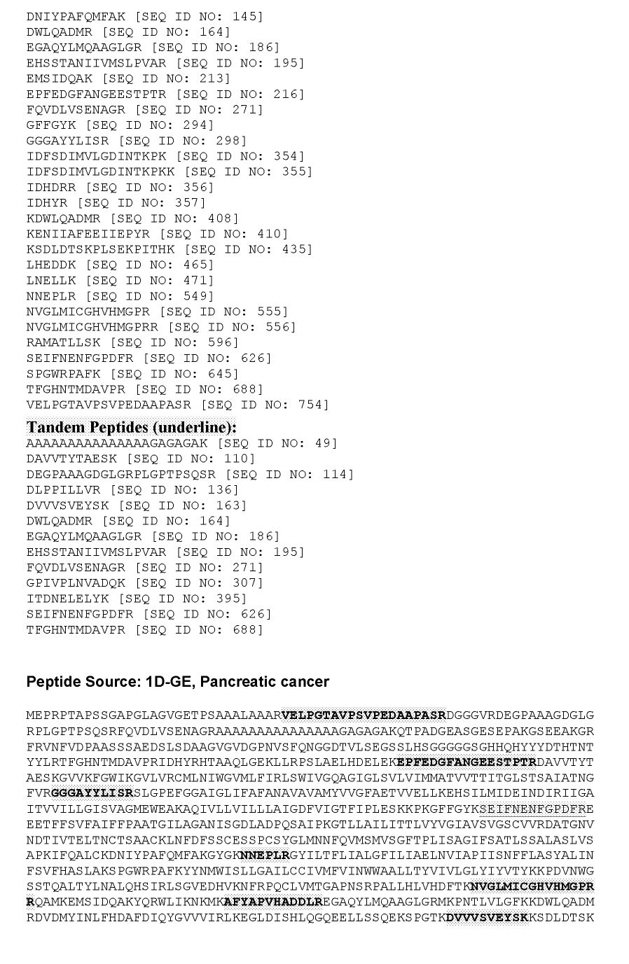

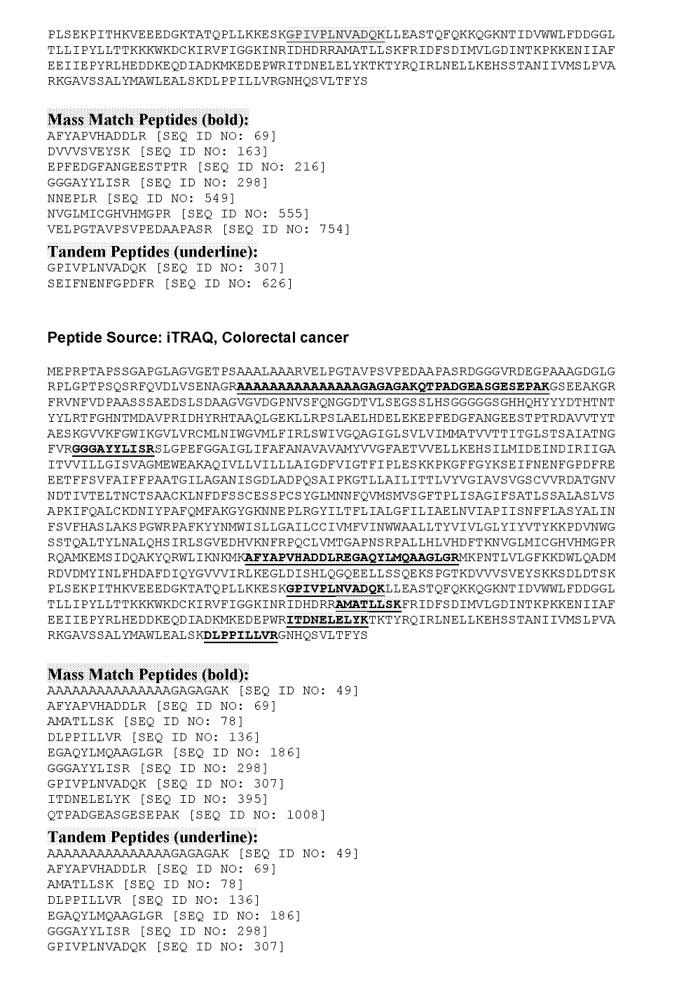

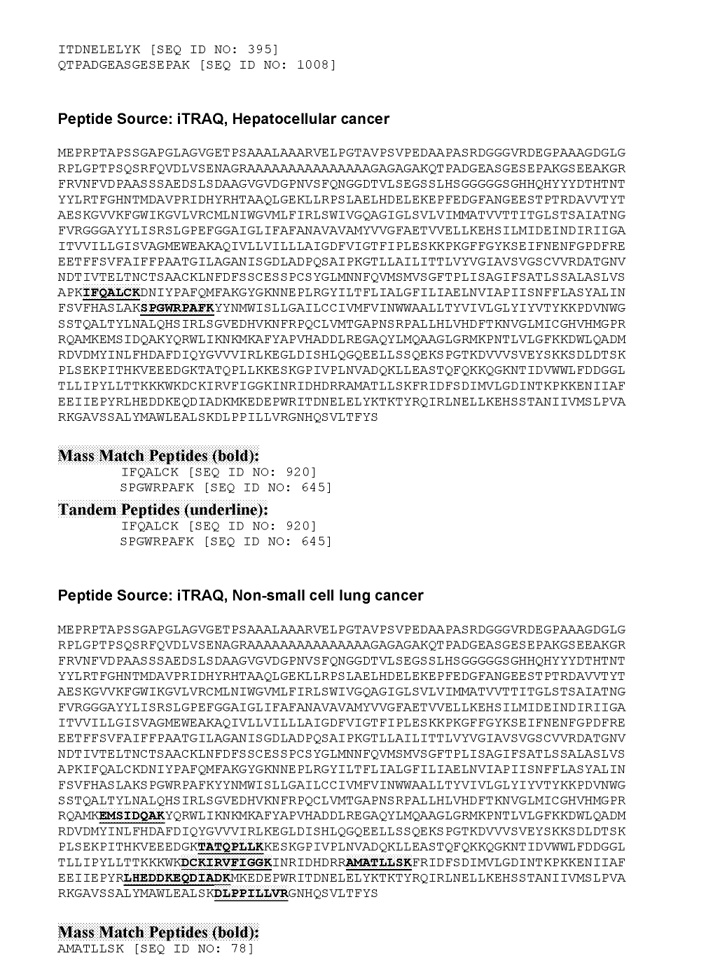

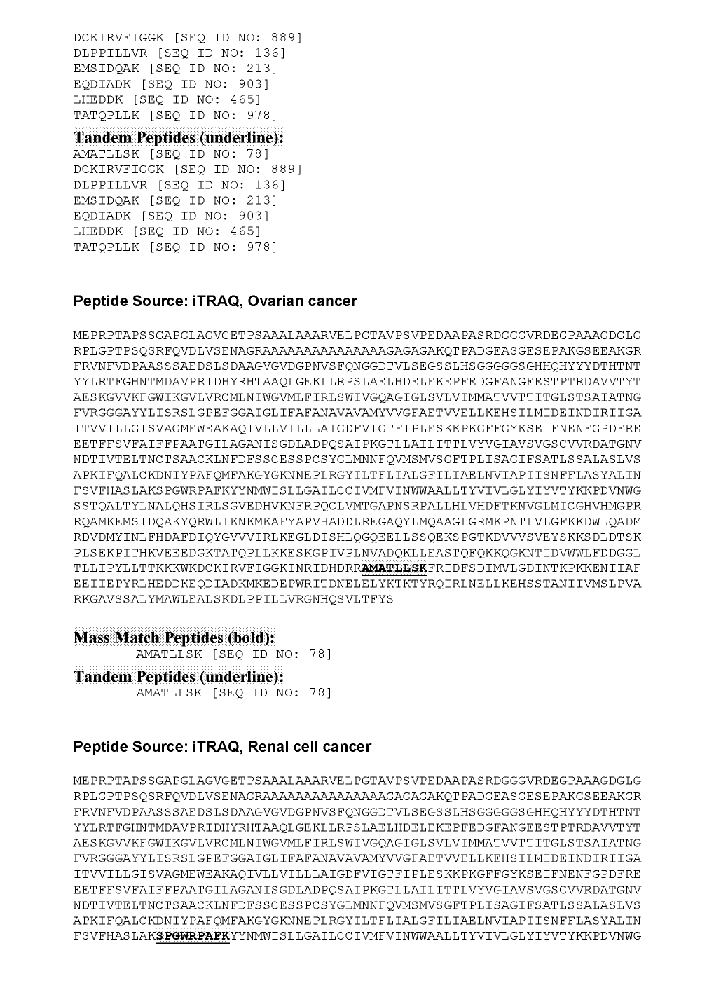

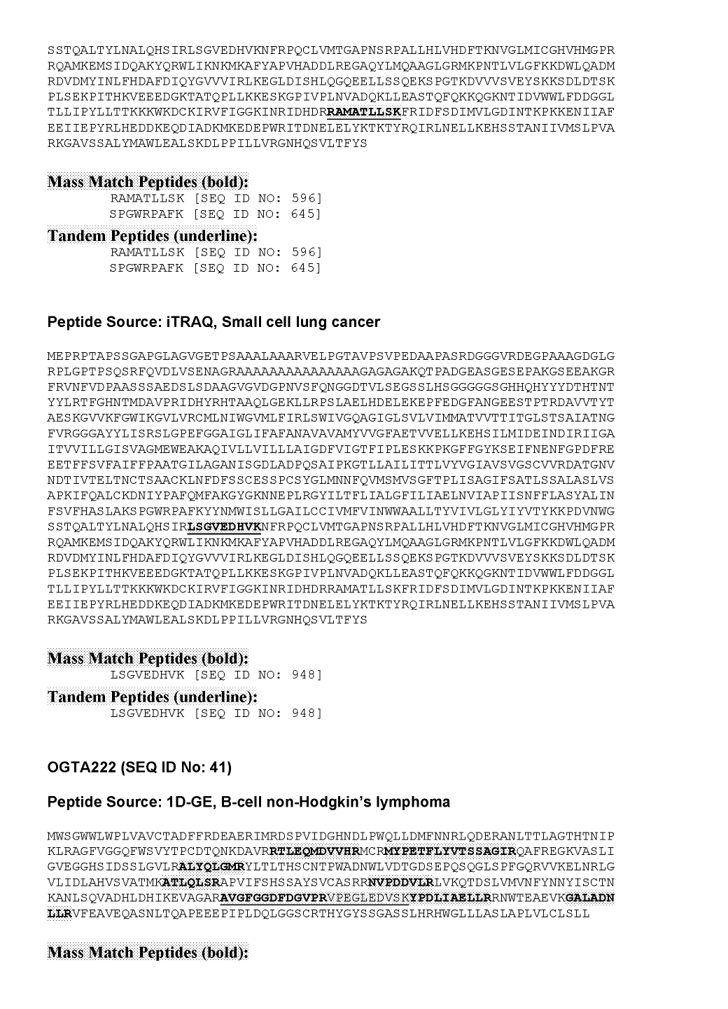

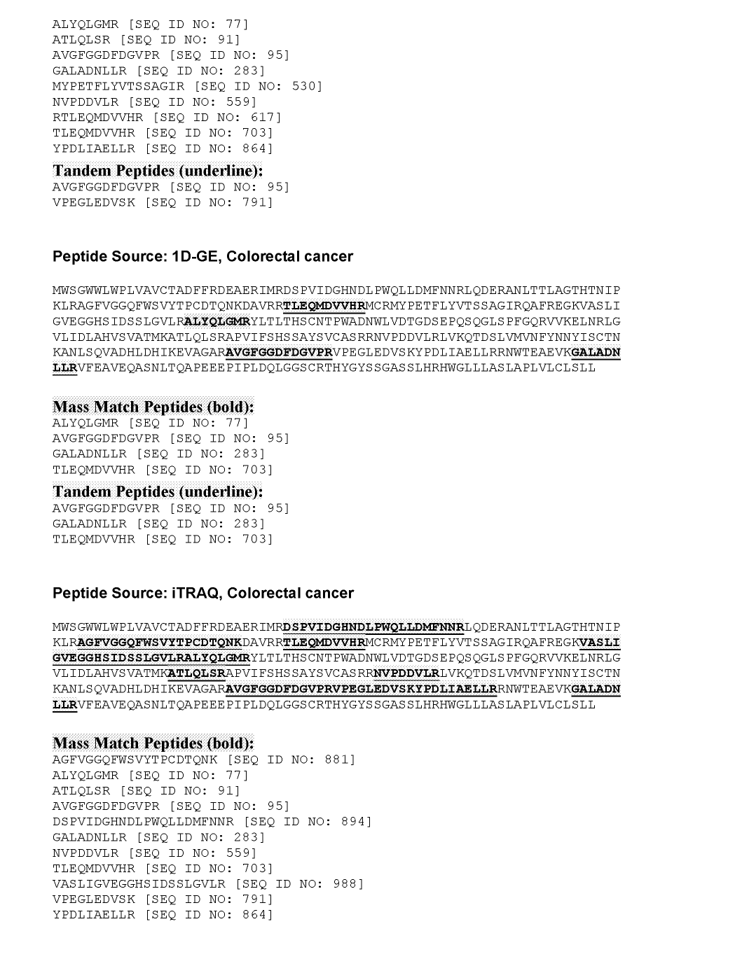

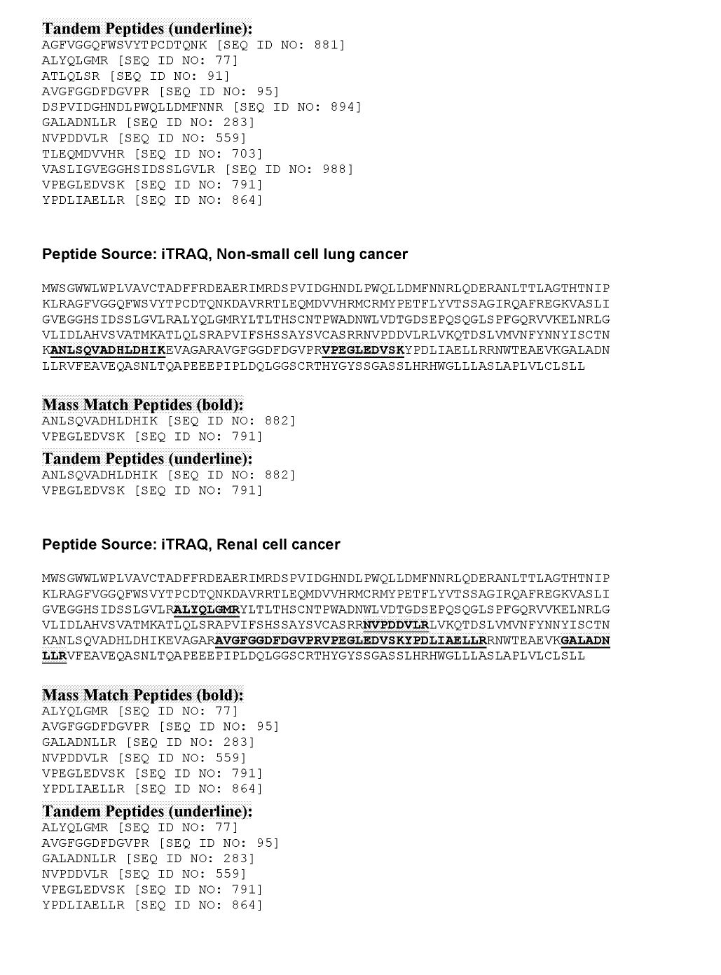

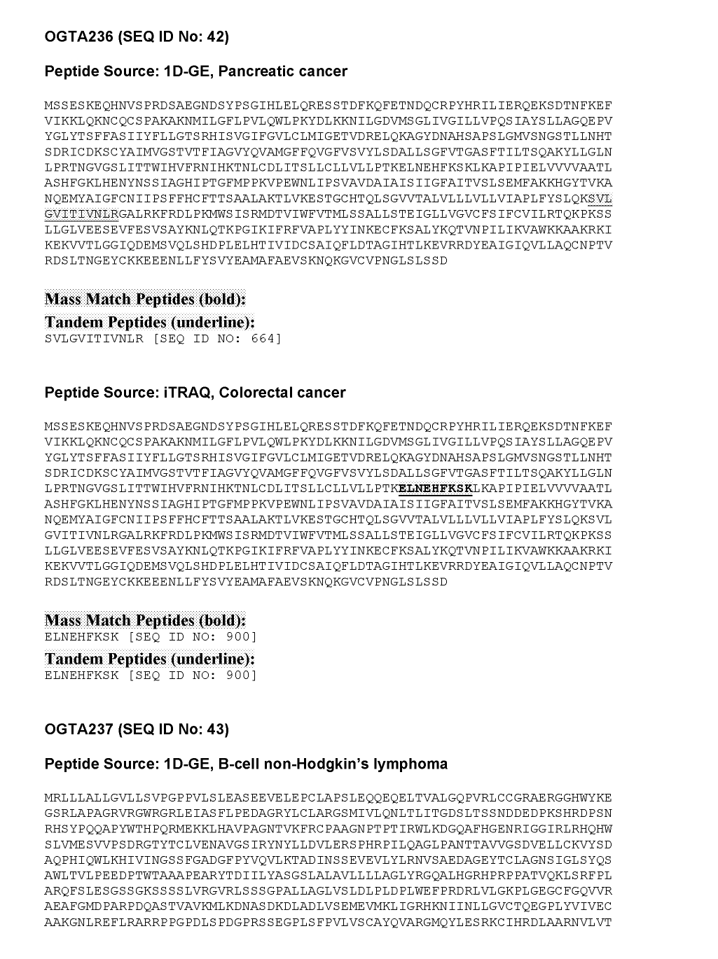

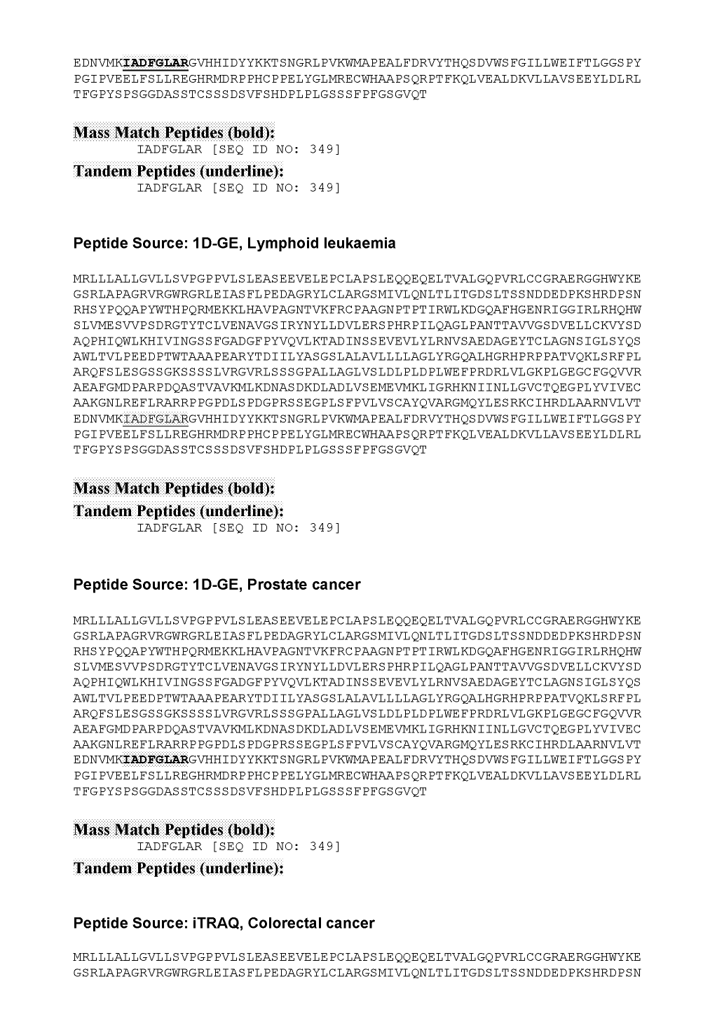

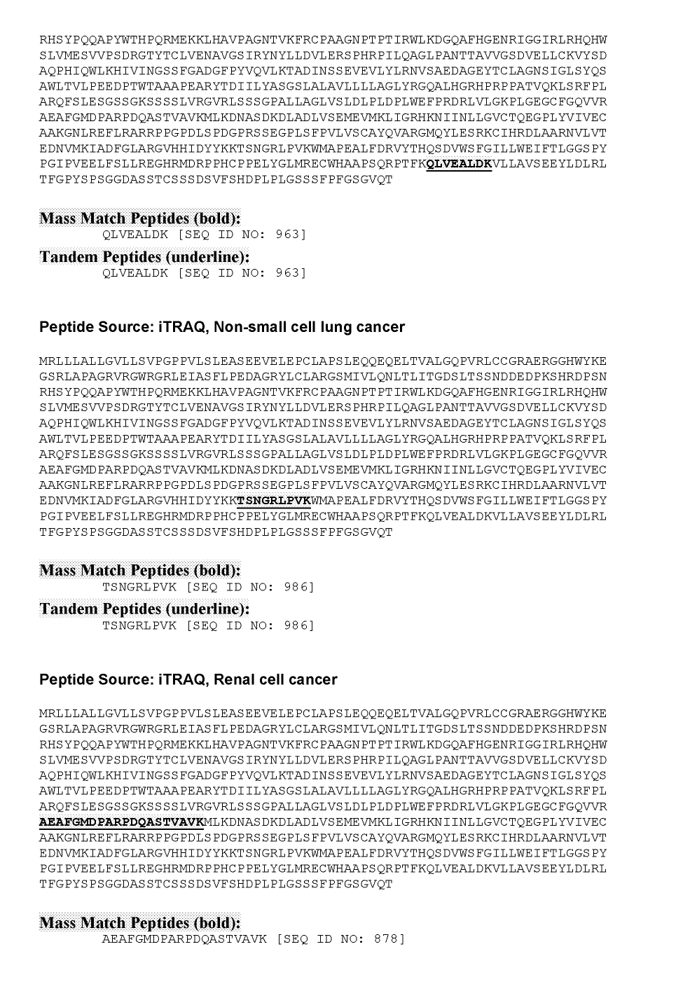

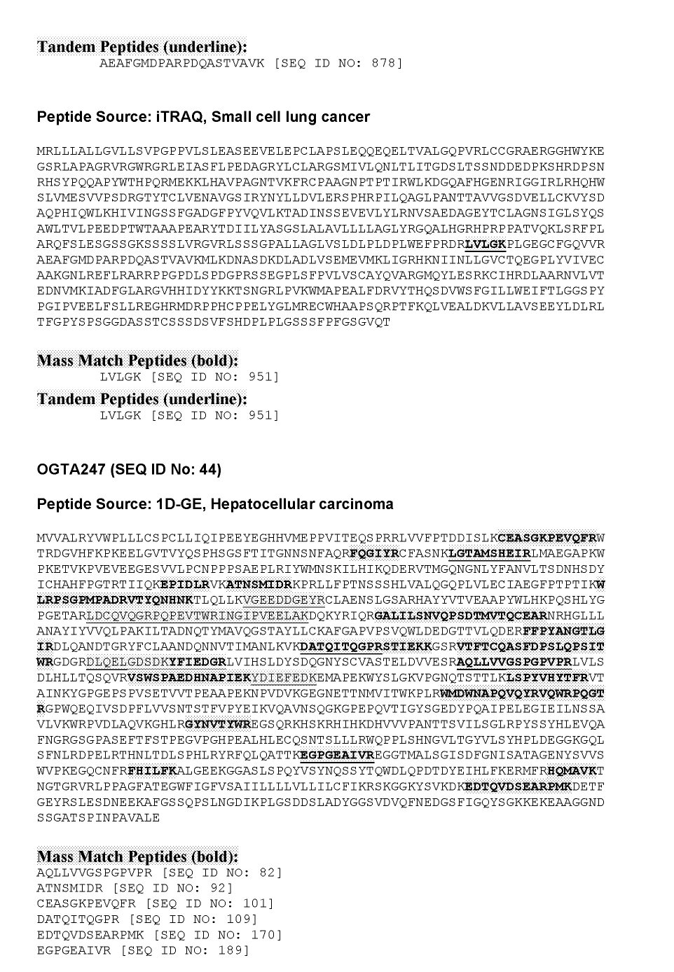

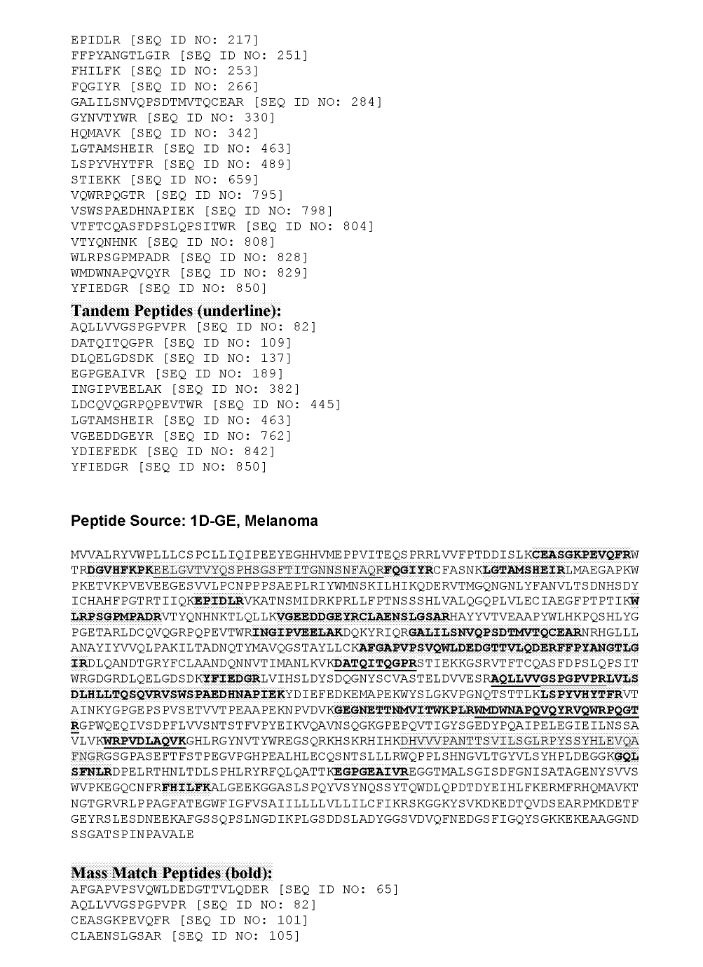

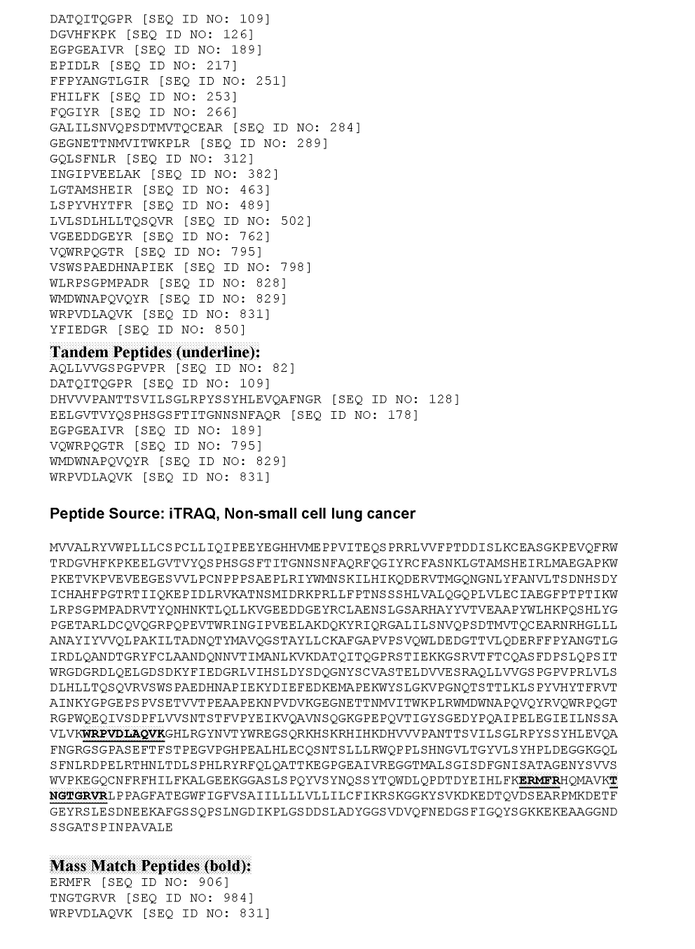

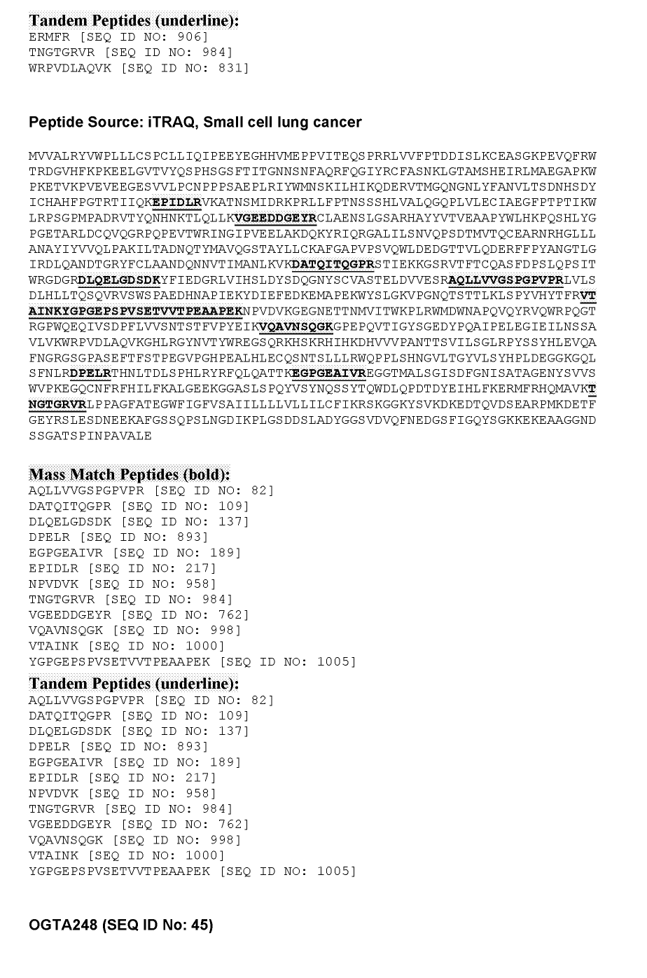

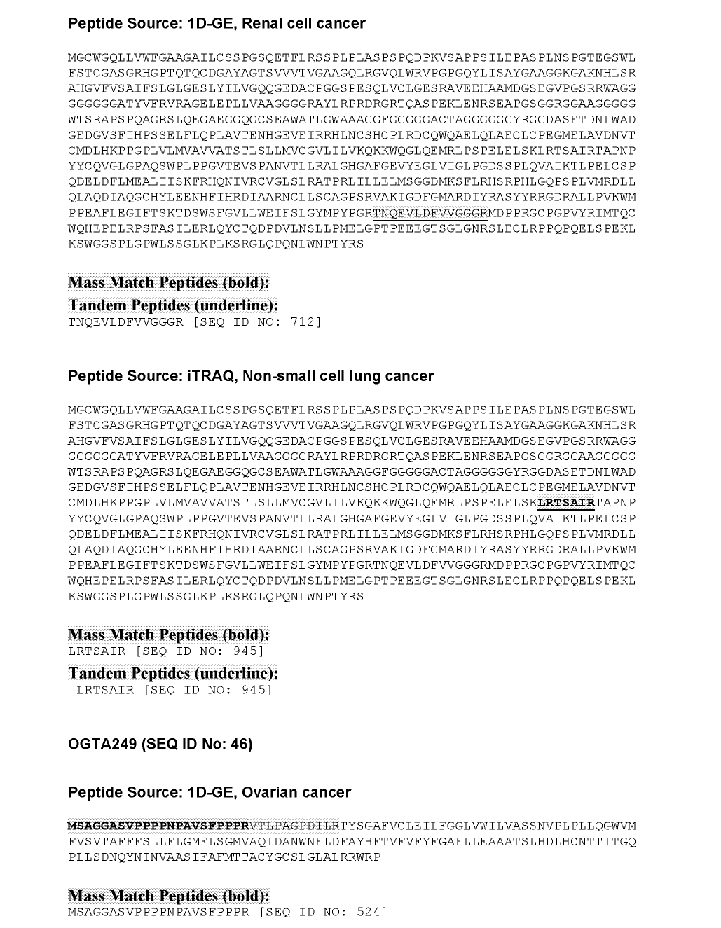

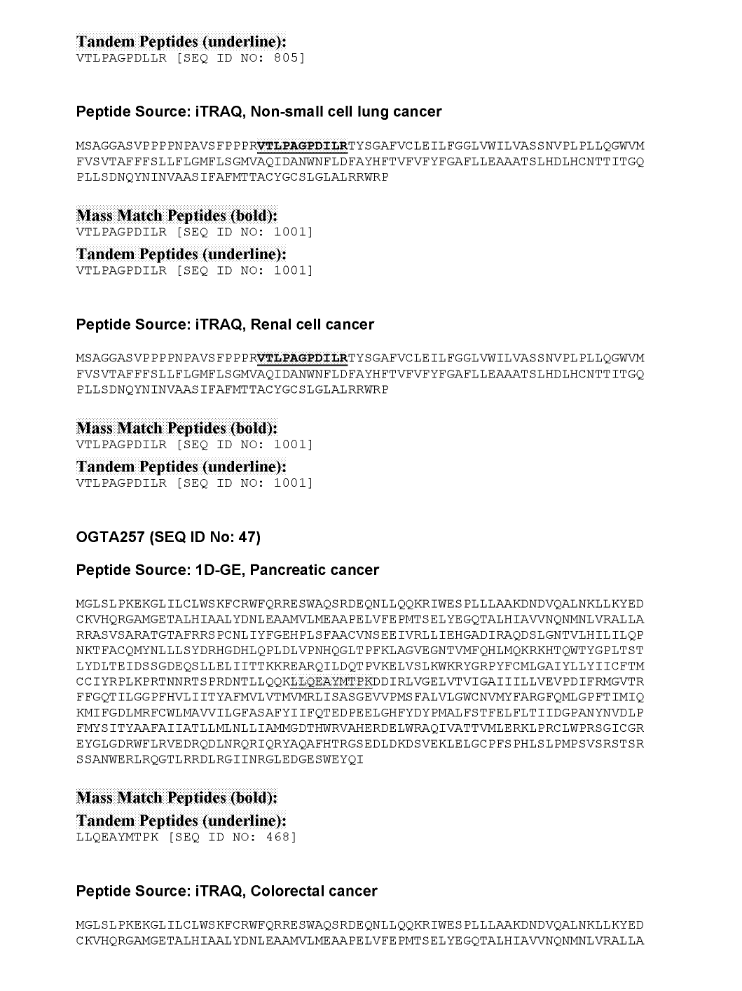

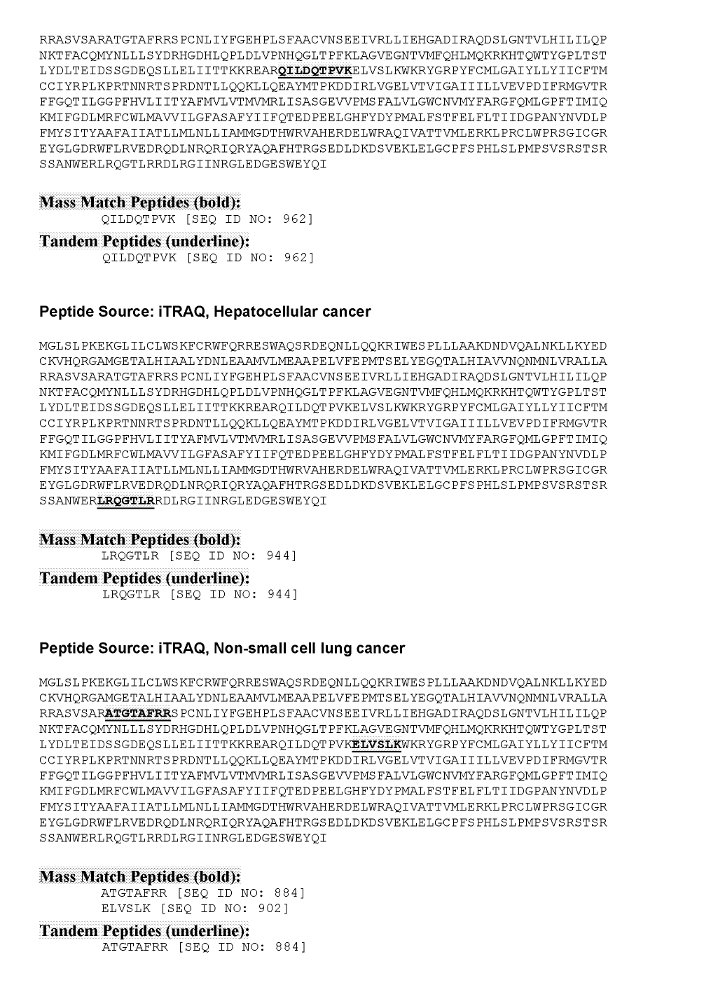

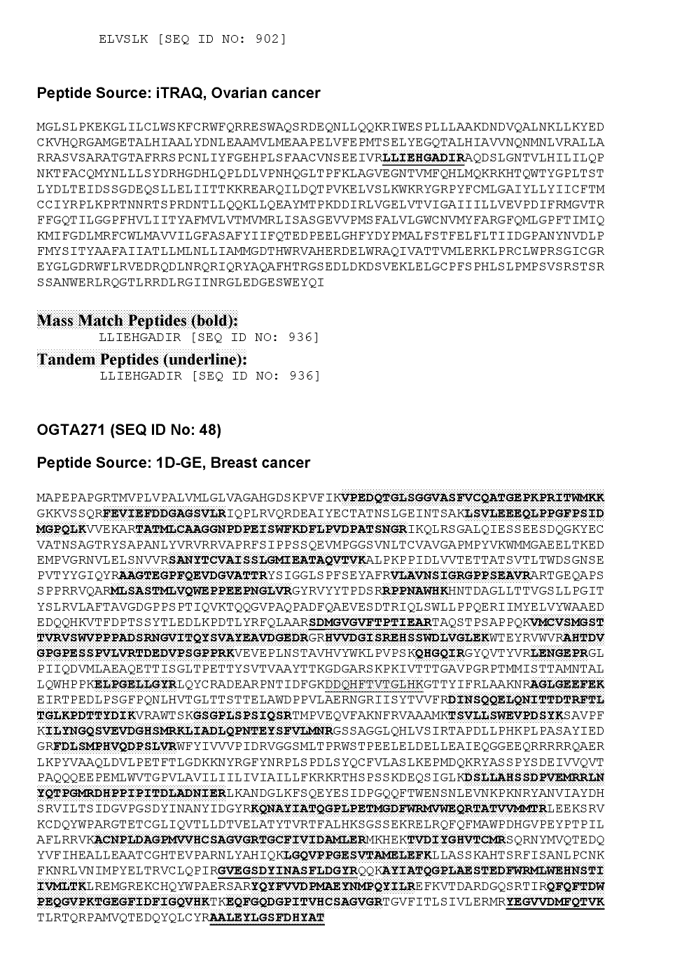

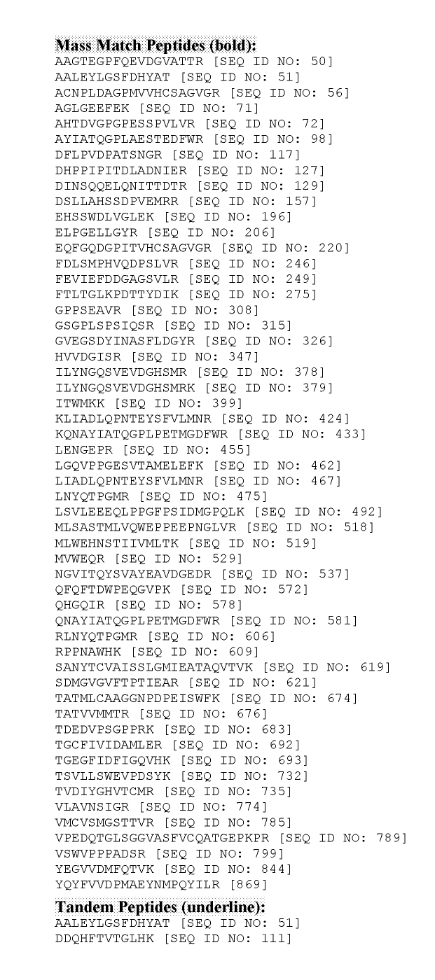

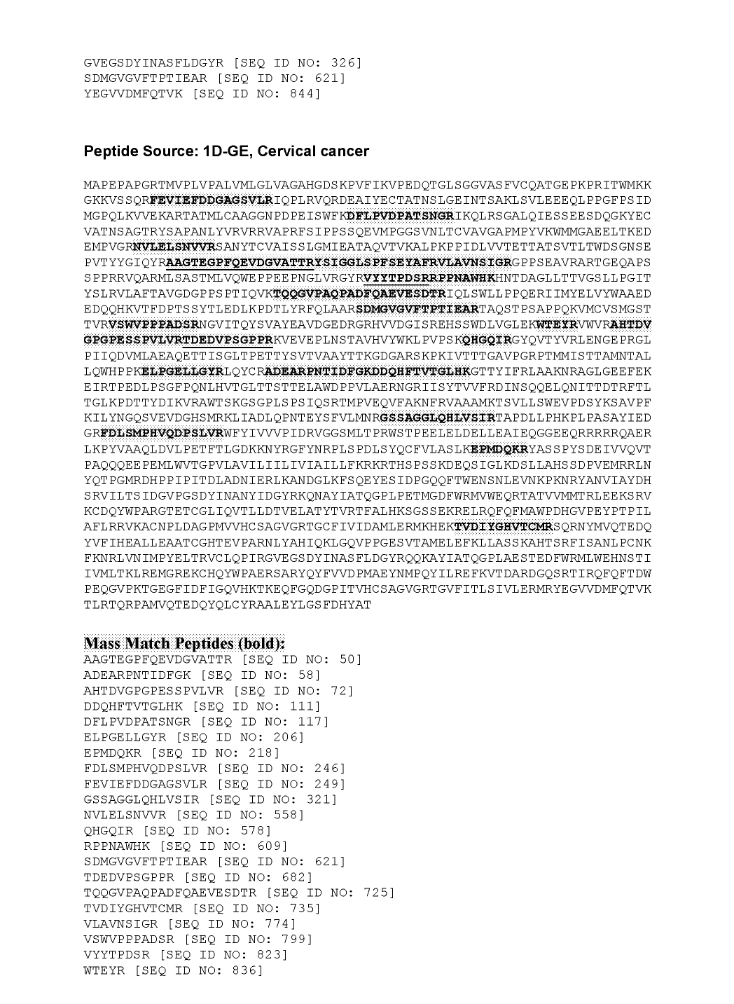

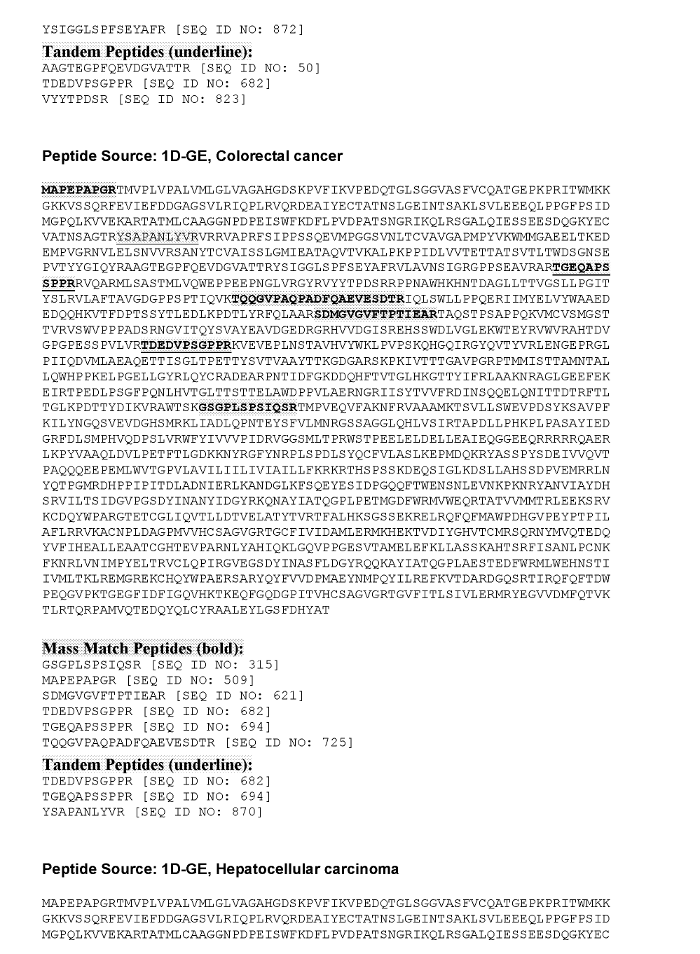

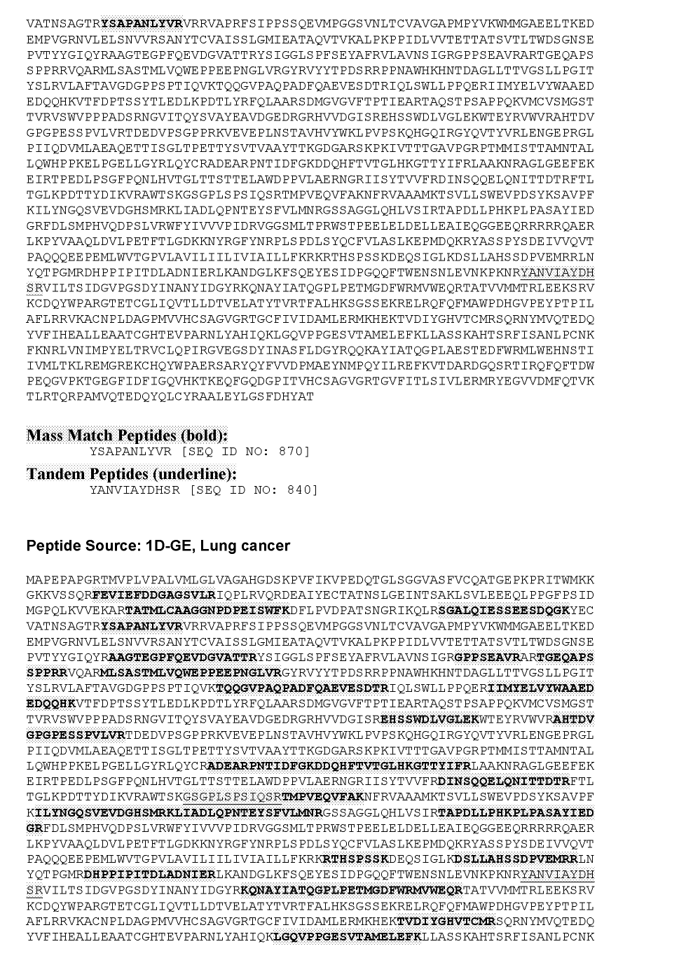

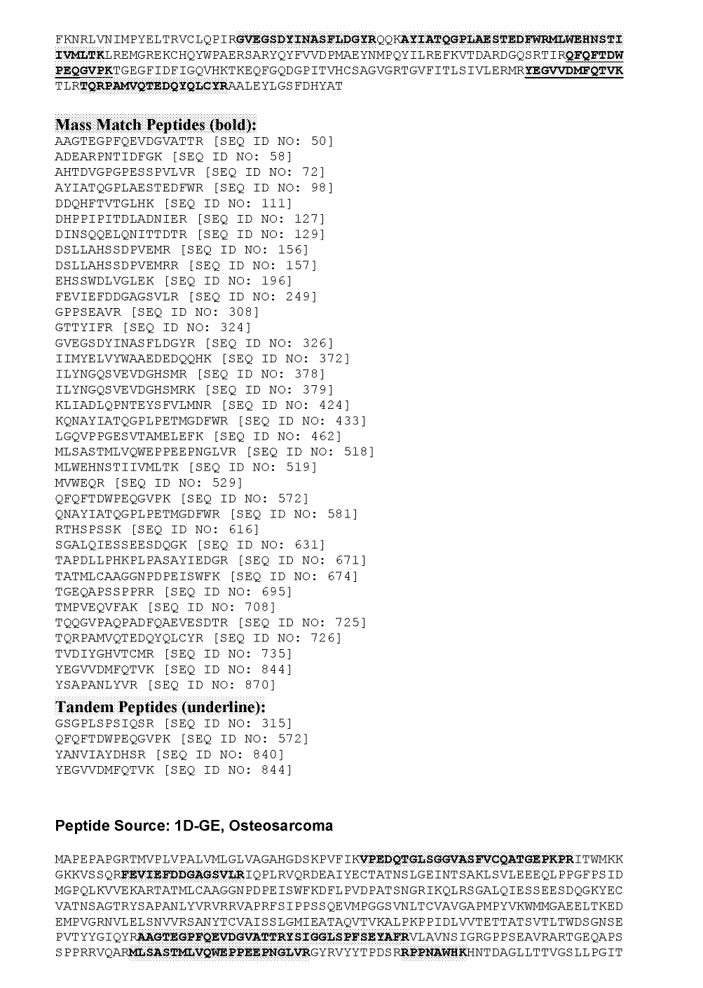

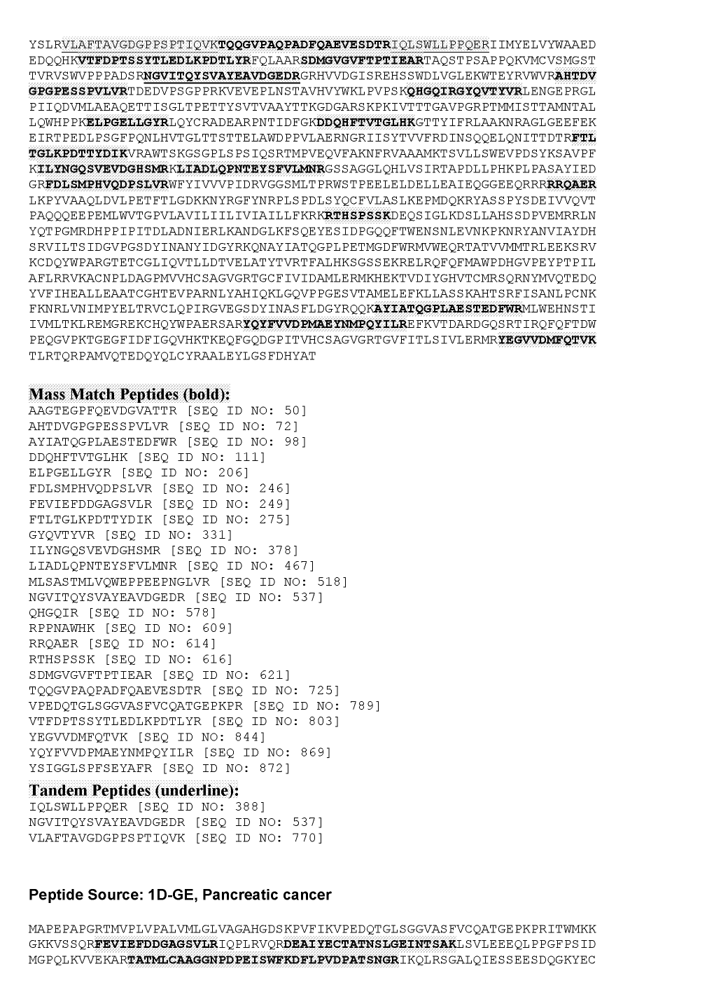

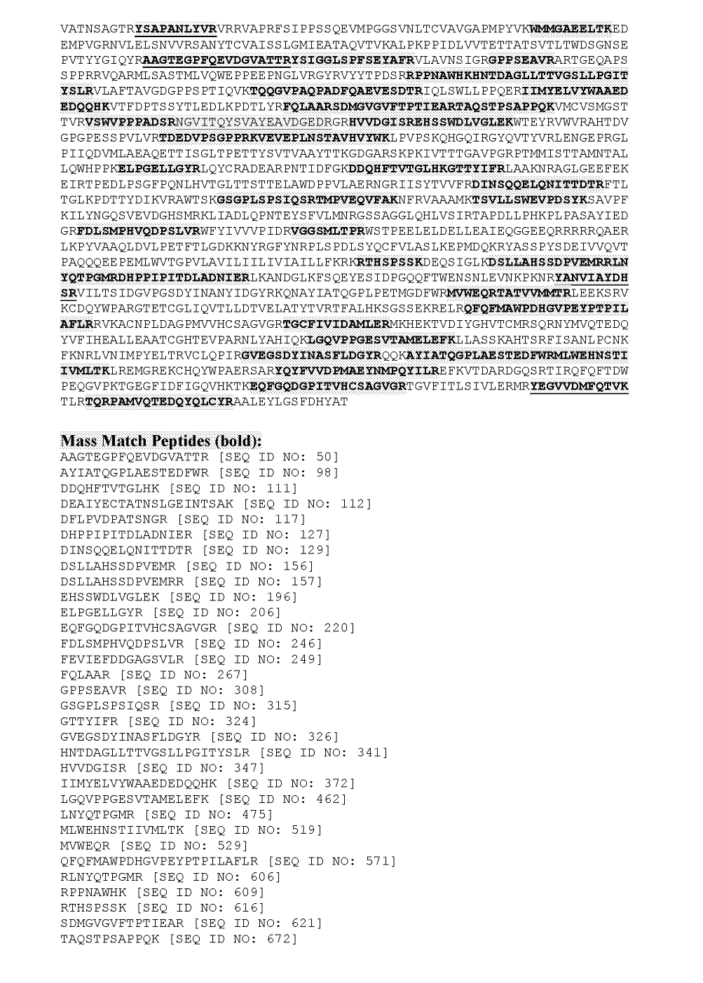

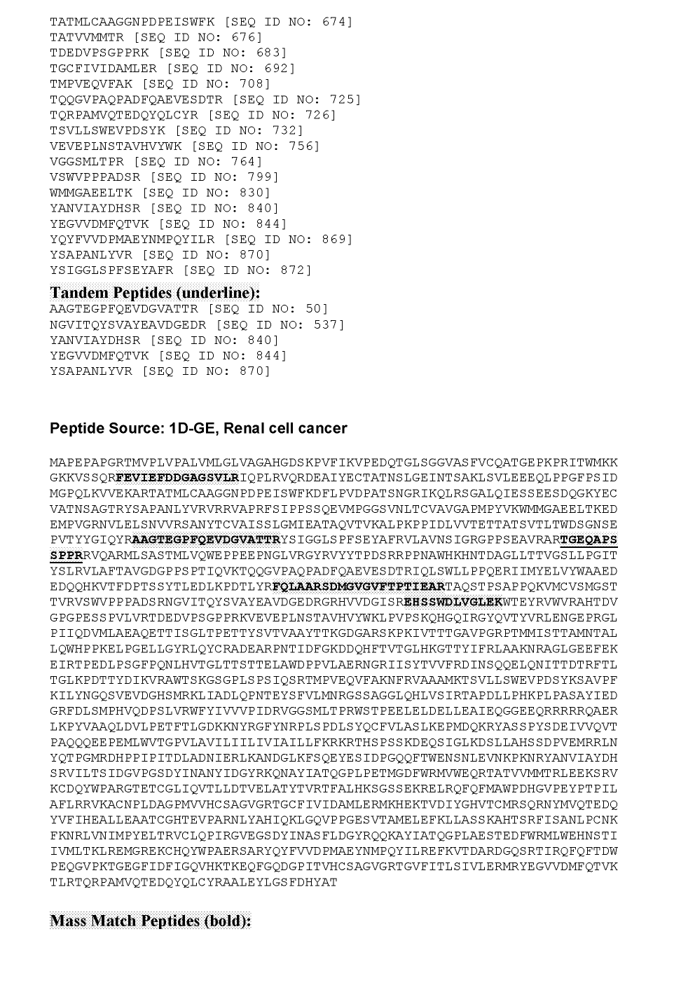

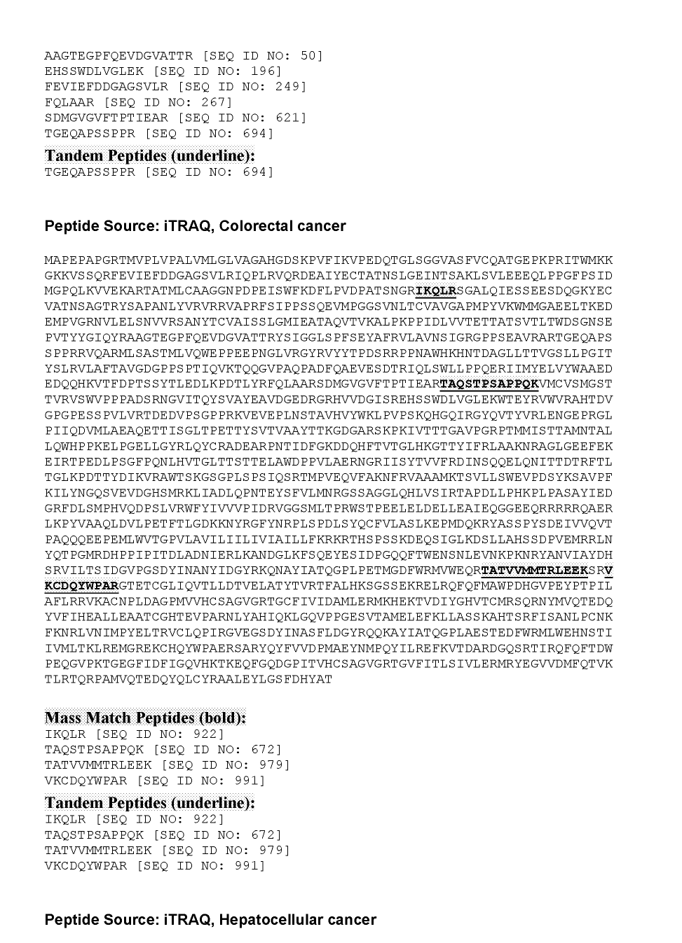

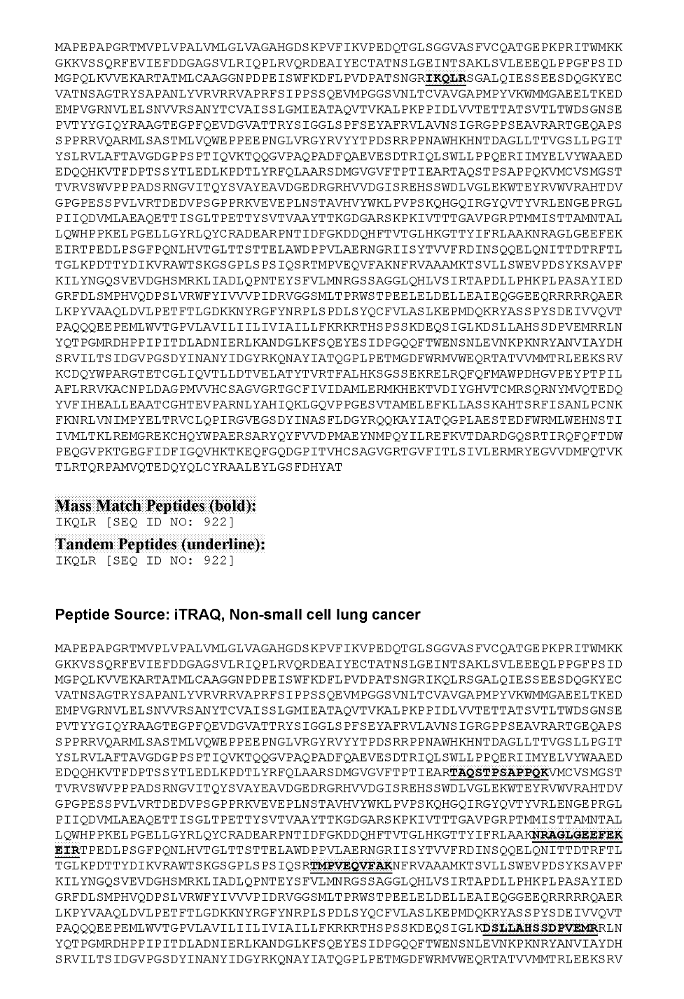

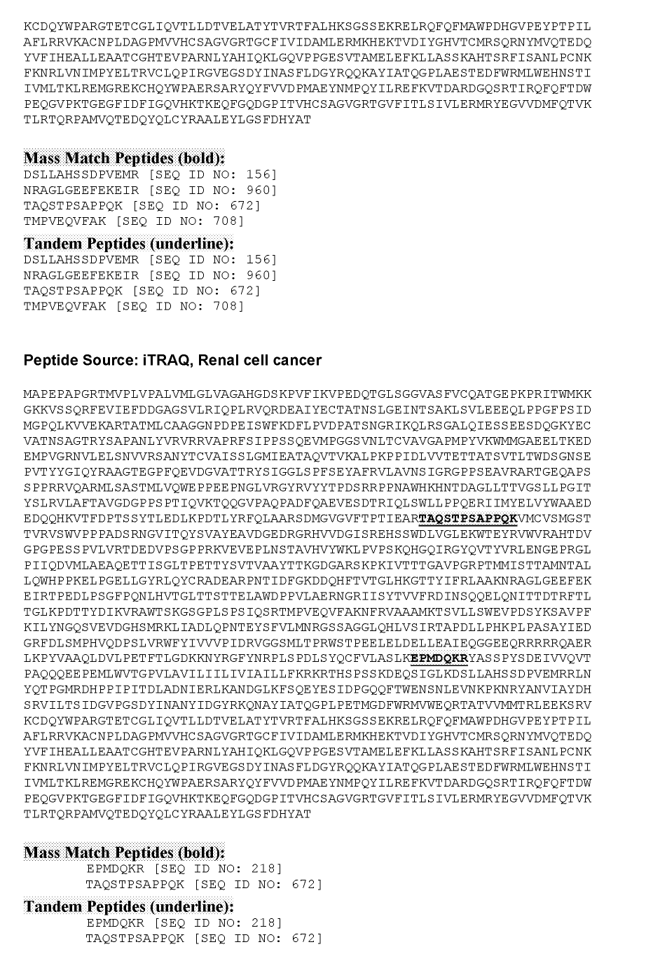

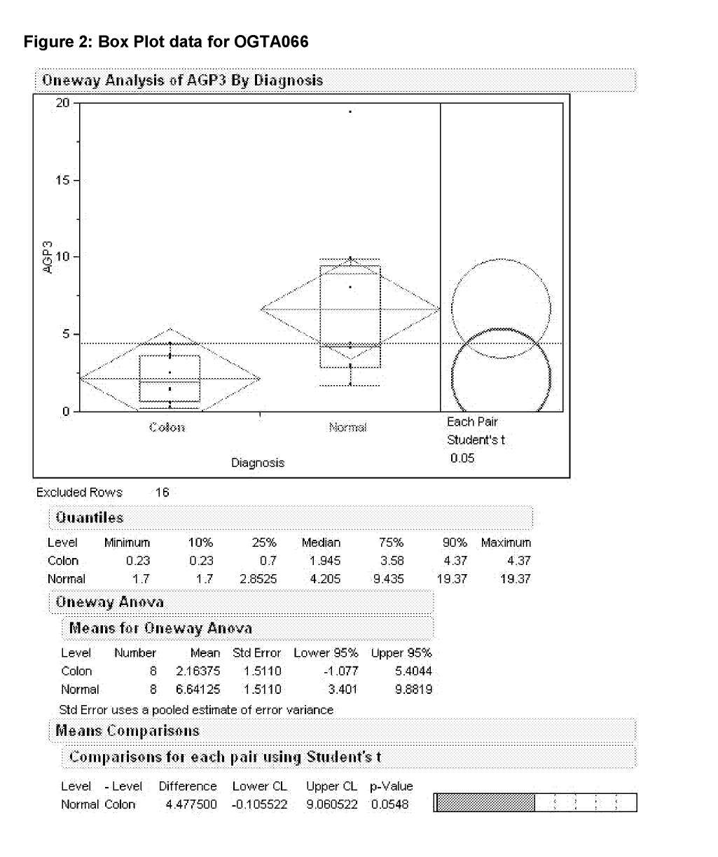

FIG. 2 shows Box plot data for OGTA066. The vertical axis on this graph is concentration of OGTA066 in ng/ml. These data show decreased concentration of OGTA066 in colorectal cancer samples compared to normal samples, with an almost significant p value, thereby indicating that OGTA066 discriminates well between colorectal cancer and normal, making it a good potential marker for colorectal cancer.

### Example 4

### Evaluation of Colorectal Cancer Marker Proteins in Multiplex Assay Using Luminex Technology

Using the following Reference Protocol, OGTA066 was evaluated in a multiplex assay using the Luminex technology.

### 4.1 Materials and Methods

Each primary antibody was conjugated to a unique Luminex magnetic microsphere (Mug beads, Luminex Corporation, Austin, Tex.). Mag bead cocktail (50 ul) was added to a 96 black well round bottom Costar plate (Corning Incorporated, Corning N.Y.). Using a 96 well magnetic ring stand, the Mag beads were pulled down for 1 minute and washed with wash/assay buffer (PBS with 1% BSA and 0.02% Tween 20). 50 ul of sample or standard was added along with an additional 50 ul of wash/assay buffer and allowed to incubate on a shaker for 1 hour at room temperature. Plate was placed on magnetic ring stand and allowed to sit for 1 minute. Mag beads were then washed again. Biotin labeled antibody was then added at 50 ul per well with an additional 50 ul of wash/assay buffer and allowed to incubate on a shaker for 1 hour at room temperature. The plate again was placed on a magnetic stand and the Mag beads were washed. Streptavidin-RPE (Prozyme, San Leandro, Calif., Phycolin, Code#PJ31S) was diluted to 1 ug/ml in wash/assay buffer and 50 ul was added to each well along with an additional 50 ul of wash/assay buffer and allowed to incubate on a shaker for 1 hour at room temperature. Final wash was performed and the beads were re-suspended with 100 ul of wash/assay buffer and each well was then read in a Luminex 200 reader using Xponent software 3.0. All reagent dilutions were made in wash/assay buffer. Biotin-antibody varied for each assay to optimal concentration. Initial Mag bead amounts added were approximately 50,000 for each assay. Magnetic beads were allowed 1 minute pull down time prior to each wash. Each wash step was 3 times washed with 100 ul of wash/assay buffer. Assay standard curves were made in a normal donor patient serum pool. Luminex reader and Mag beads were used and prepared according to manufacturer guidelines. Standard curves were calculated using a 5 parameter log-logistic fit and each sample concentration was determined from this curve fit.

Final Box and ROC results were analyzed using Analyse-it General+Clinical Laboratory 1.73 (Analyse-it Software Ltd., Leeds England).

### 4.2 Results

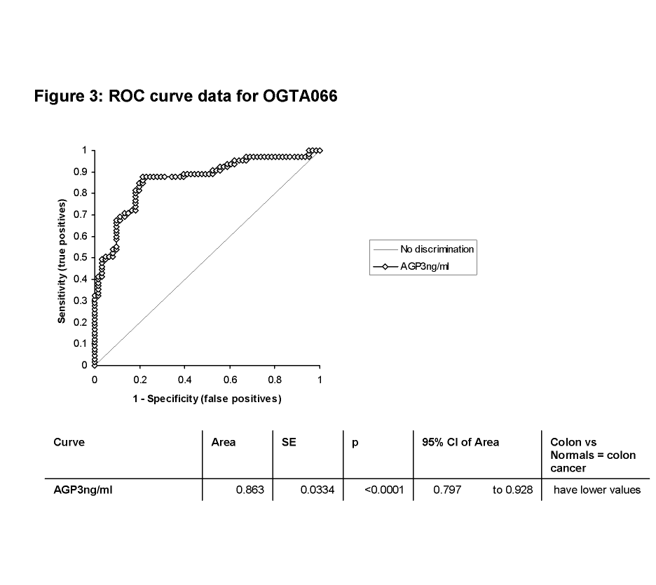

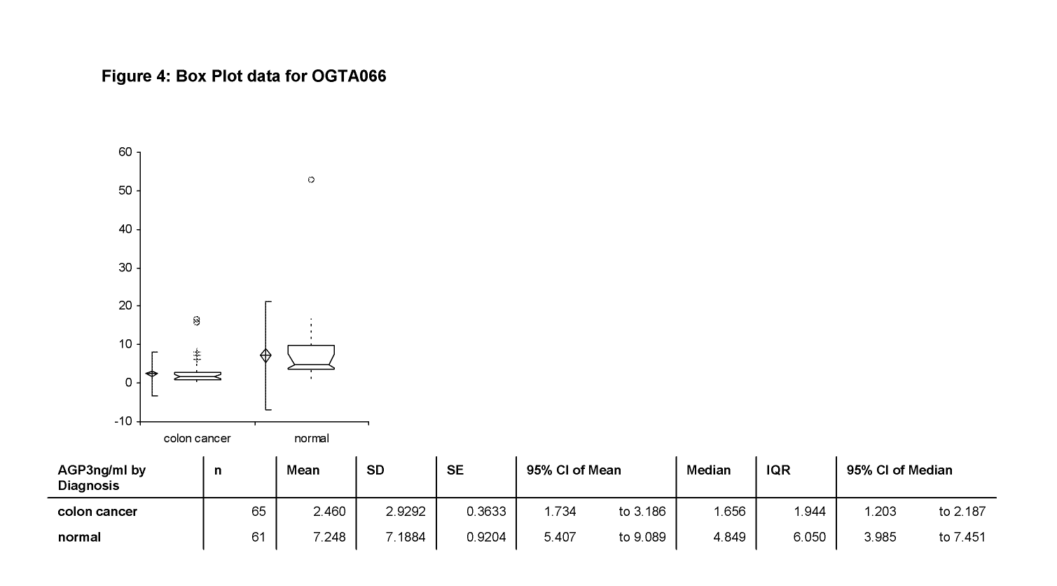

Experiments using 61 normal samples and 65 colorectal cancer samples resulted in further evidence for some of the proteins of interest identified in Example 3 above, including, but not limited to, OGTA066. FIG. 4 shows Box plot data for OGTA066. FIG. 3 shows ROC curve data for OGTA066.

The ROC curves plot sensitivity (true positives) against 1-specificity (false positives). The area under the ROC curve is a measure of the probability that the measured marker level will allow correct identification of a disease or condition. An area of greater than 0.5 indicates that the marker can discriminate between disease and normal. This is the case in the data shown in FIG. 3, with OGTA066 having a high area under the curve and a very low p value therefore indicating that OGTA066 is a good potential marker to discriminate between colorectal cancer and normal.

The vertical axes on the box plot in FIG. 4 is concentration of the OGTA066 in ng/ml. FIG. 4 shows lower concentration in serum samples of OGTA066 in colorectal cancer samples than in normal samples. OGTA066 shows good discrimination between colorectal cancer and normal, indicating it may be useful as a marker for colorectal cancer.

### Example 5

### Identification of CDH3 Expressed in Breast Cancer and Ovarian Cancer Tissue Samples Using Liquid Chromatography-Mass Spectrometry (Lc/Ms)

Using the following protocol, membrane proteins extracted from breast cancer and ovarian cancer tissue and corresponding normal or normal adjacent tissue (NAT) samples were digested and resulting peptides sequenced by tandem mass spectrometry.

### 1.1 Materials and Methods

### 1.1.1 Plasma Membrane Fractionation

The cells recovered from a breast cancer and ovarian cancer or a normal or normal adjacent tissue were homogenised and submitted to centrifugation at 1000×g. The supernatant was taken and ultra-centrifuged at 49500×g. The resulting pellet was re-homogenized and separated by discontinuous sucrose density centrifugation. After ultra-centrifugation at 107000×g, the fractions at the phase boundary were recovered and pelleted.

### 1.1.2 Plasma Membrane Solubilisation

Plasma membrane fractions were resuspended in SDS (Sodium dodecyl sulfate) to give a final SDS concentration of 0.5%, centrifuged and the solubilized protein extracted.

### 1.1.3 Trypsinolysis

For in-solution digestion, the volume of a 50 μg protein solution was made up to 100 μl using 200 mM ammonium bicarbonate. 10 μl of the reducing agent DL-Dithiothreitol (75 mM) was added to the sample and incubated at 80° C. for 15 minutes. This was followed by a cysteine blocking step using 10 μl of 150 mM iodoacetamide and incubation in the dark for 30 minutes at room temperature. The SDS concentration was then diluted to 0.05% with the addition of ultra-pure water. A sufficient volume of trypsin (Promega V5111) was added to the mixture allowing for 1 μg of trypsin to 2.75 μg of protein and incubated overnight at 37° C. Alternatively, 105 μg of protein solutions were reduced using 3 μl of 50 mM TCEP and incubating at 60° C. for 1 hr. The sample was then processed on the FASP filtration devices of the Protein Digestion Kit (Protein Discovery) according to the manufacturer's instructions, but using triethylammonium bicarbonate instead of ammonium bicarbonate. Trypsinolysis was performed in a final volume of 75 μl, using 1 μg of trypsin to 50 μg of protein.

### 1.1.4 Peptide Fractionation

The digested protein samples were dried under a vacuum, re-suspended in 0.1% aqueous formic acid and trifluoroacetic acid (TFA) was added to reduce the pH of the solution to <3. Peptides were separated by ion exchange using an Agilent Zorbax Bio-Strong Cation Exchange series II column on an Agilent LC1200 Series liquid chromatography system. Alternatively, the Agilent 3100 OFFGEL Fractionator and the OFFGEL Kit pH 3-10 was used for pI-based separation, according to the protocol of the supplier. Following re-hydration of the IPG strips, equal volumes of a membrane digest were loaded into each well. Following separation, the resulting fractions were acidified.

### 1.1.5 Mass Spectrometry

Fractionated samples were analysed by liquid chromatography-mass spectrometry using a Waters nanoACQUITY UPLC System fitted with a nanoACQUITY UPLC BEH 130 C18 column, 75 μm×250 mm (186003545) and a LTQ Orbitrap Velos (Thermo Fisher Scientific). Peptides were eluted with a 300 nl/min gradient increasing from 3% to 35% acetonitrile over 120 min. Full-scan mass spectra were acquired at 60000 resolving power between 400-2000 m/z mass range in the Orbitrap. In each cycle, the twenty most intense peptides were selected for CID MS/MS scans in the linear ion trap with nanospray ion source fitted on the instrument.

### 1.1.6 Amino Acid Sequence Analysis of Peptide

The raw data generated from the LTQ Orbitrap Velos was processed through the Mascot software (Matrix Science) which uses the Mowse algorithm (Curr Biol. 1993 Jun. 1; 3(6):327-3) to infer amino acids sequences from the peak lists by searching against a sequence database consisting of Ensembl (worldwide web.ensembl.org/index.html), IPI (worldwide web ebi.ac.uk/IPI/IPIhuman.html) and SwissProt (worldwide web uniprot.org) along with contaminant protein sequences. Criteria for peptide identification included trypsin digestion, up to 2 missed cleavage sites and various biological and chemical modifications (oxidized methionine, cysteine modification by MMTS or iodoacetamide and phosphorylation of serine, threonine and tyrosine). Peptides ranked 1 with an expectation value of 0.05% or less, an ion score of 28 or higher were loaded into our OGAP database where they were processed into protein groups.

### 1.1.7 Discrimination of Breast Cancer and Ovarian Cancer Associated Proteins

The process to identify CDH3 (SEQ ID No: 35) used the peptide sequences obtained experimentally by mass spectrometry, as described above, of naturally occurring human proteins to identify and organize coding exons in the published human genome sequence. These experimentally determined sequences indicated in Table 94, were compared with the OGAP® database which was compiled by processing and integration of peptide masses, peptide signatures, ESTs and Public Domain Genomic Sequence Data as described in International Patent Application WO2009/087462.

TABLE 94
CDH3 Specific Peptides Identified By LC/MS in
the plasma membranes of breast cancer and
ovarian cancer tissue samples.

| SEQ ID No | Peptide Identified |
|---|---|
| SEQ ID No: | ETGWLLLNKPLDREEIAK |
| 1009 | |
| SEQ ID No: | FTQDTFR |
| 277 | |
| SEQ ID No: | DPHDLMFTIHR |
| 1010 | |
| SEQ ID No: | STGTISVISSGLDR |
| 1011 | |
| SEQ ID No: | YEAHVPENAVGHEVQR |
| 1012 | |
| SEQ ID No: | LTVTDLDAPNSPAWR |
| 1013 | |
| SEQ ID No: | ATYLIMGGDDGDHFTITTHPESNQGILTTR |
| 1014 | |
| SEQ ID No: | NQHTLYVEVTNEAPFVLK |
| 1015 | |
| SEQ ID No: | QITICNQSPVR |
| 1016 | |
| SEQ ID No: | FLKQDTYDVHLSLSDHGNK |
| 1017 | |
| SEQ ID No: | QDTYDVHLSLSDHGNK |
| 1018 | |
| SEQ ID No: | IKEPLLLPEDDTR |
| 1019 | |
| SEQ ID No: | GLEARPEVVLR |
| 1020 | |

### 1.1.8 Protein Index

The protein index is a measure of both protein prevalence and peptide abundance. The algorithm takes into account both the number of samples in which the protein has been observed and the number of peptides observed vs observable peptides from each sample. The resulting value is then graded by pairwise comparison of corresponding normal samples vs cancer samples.

### 1.2 Results

These experiments identified CDH3 as further described herein. The full-length CDH3 was detected in the plasma membrane of breast cancer and ovarian cancer tissue samples. Table 2 shows the expression distribution of CDH3 measured by the protein index. Expression of CDH3 in these cancer tissues indicates CDH3 is a valuable therapeutic and diagnostic target in these cancers.

TABLE 95
CDH3 Protein Index

| Tissue | Cancer | Normal |
|---|---|---|
| Breast | ++ | — |
| Ovarian | +++ | — |

(+++++ = Very High; ++++ = High; +++ = Medium; ++ = Low; + = Very low; — = Not Observed)

### Example 6

### Immunohistochemistry Using Antibody to CDH3

Using the following Reference Protocol, immunohistochemistry was performed on FFPE tumor and normal tissues using a polyclonal antibody to CDH3 (Atlas Antibodies, Sweden).

### 2.1 Materials and Methods

### 2.1.1 Materials

Citroclear (HC5005) from TCS Biosciences, UK.
Reagent alcohol (R8382) from Sigma-Aldrich, UK.
Target Retrieval Solution, pH6 (S2369) from Dako, UK.
REAL Peroxidase Blocking Solution (S2023) from Dako, UK
Antibody Diluent (S0809) from Dako, UK
EnVision+HRP-conjugated polymer, Rabbit (K4002) from Dako, UK.
Liquid DAB+substrate (K3468) from Dako, UK.
Mayer's Hematoxylin (X0909) from Dako, UK
Aquatex (1.08562.0050) from VWR, UK

Tissue sections and arrays were from US Biomax Inc., MD, USA.

### 2.1.2 Deparaffinisation and Rehydration

Slides were deparaffinised in Citroclear (2×5 minutes) then rehydrated through 100% alcohol (2×5 minutes), 50% alcohol (1×5 minutes) and tap water (1×5 minutes).

### 2.1.3 Antigen Retrieval (Pressure Cooker)

The CDH3 antigen was retrieved using microwave heat for 10 minutes in 50 ml Target Retrieval Solution in a Coplin jar. Slides were then left to cool to room temperature for a further 20 min. Circles were drawn around each tissue section/TMA with a hydrophobic barrier pen and slides were then washed twice in PBS, 3 minutes each wash.

### 2.1.4 Tissue Staining

Endogenous peroxidase activity was blocked by incubating tissues with Peroxidase Blocking Solution for 10 minutes at RT in a humidified chamber. Slides were then washed once in PBS and once in PBS-T (PBS containing Tween-20, 0.125% v/v), 3 minutes each wash. Primary antibody (at a concentration of 0.28 ug/ml) was applied to each tissue section and/or microarray, and the slides were incubated for 45 min at room temperature in a humidified chamber. Slides were then washed once in PBS and once in PBS-T, 3 minutes each wash. The EnVision+HRP-conjugated polymer was then applied to the tissues and the slides were incubated for 30 min at room temperature in a humidified chamber. Slides were then washed once in PBS and once in PBS-T, 3 minutes each wash. Tissues were incubated in Liquid DAB+substrate at room temperature for 10 min in a humidified chamber. Slides were then washed once in PBS and once in PBS-T, counterstained with Hematoxylin for 1 min at room temperature in a humidified chamber, and washed again, once in PBS and once in PBS-T, 3 minutes each wash. Coverslips were then mounted onto the slides using Aquatex.

### 2.2 Results

Immunohistochemical analysis revealed specific staining of tumor cells in breast cancer, including specific staining of tumor cells in triple negative breast cancer. At high magnification it was evident that most of the cells were heavily stained in the plasma membrane. Thus antibodies directed to CDH3 may have utility as therapeutics and diagnostics in these cancers and other cancer types showing expression of CDH3.

### Example 7

### Internalization and MabZAP of Anti-CDH3 Monoclonal Antibodies in A431 Cell

### 3.1 Materials and Methods

Internalization of anti CDH3 monoclonal antibodies by A431 cells were investigated using a MabZap assay. The MabZAP assay showed internalization of the anti-CDH3 monoclonal antibodies through binding of an anti-human IgG secondary antibody conjugated to the toxin saporin. (Advanced Targeting System, San Diego, Calif., IT-22-100). First, anti-CDH3 Mab was bound to the surface of the cells. Then, the MabZAP antibodies were bound to the primary antibodies. Next, the MabZAP complex was internalized by the cells. The entrance of Saporin into the cells resulted in protein synthesis inhibition and eventual cell death.

The MabZAP assay was conducted as follows. Each of the cells was seeded at a density of 5×10³ cells per well. The anti-CDH3 monoclonal antibody or an isotype control human IgG was serially diluted then added to the cells and incubated for 15 min at 25° C. The MabZAP was then added and incubated for 72 hr at 37° C. Cell viability in the plates was detected by CellTiter-Glo® Luminescent Cell Viability Assay kit (Promega, G7571) and the plates were read and analysed using Promega Glomax.

### 3.2 Results

FIG. 5 shows that the anti-CDH3 monoclonal antibodies were efficiently internalized by A431 cells, as compared to the anti-human IgG isotype control antibody. Therefore, anti-CDH3 antibodies conjugated to a toxin may have therapeutic utilities in these and other cancer cells.

### Example 8

### Identification of MUC13 Expressed in Colorectal Cancer, Gastric Cancer and Pancreatic Cancer Tissue Samples Using Liquid Chromatography-Mass Spectrometry (LC/MS)

Using the following protocol, membrane proteins extracted from colorectal cancer, gastric cancer and/or pancreatic cancer tissue and corresponding normal or normal adjacent tissue (NAT) samples were digested and resulting peptides sequenced by tandem mass spectrometry.

### 4.2 Materials and Methods

### 4.1.1 Plasma Membrane Fractionation

The cells recovered from a colorectal cancer, gastric cancer and pancreatic cancer or a normal or normal adjacent tissue were homogenised and submitted to centrifugation at 1000×g. The supernatant was taken and ultra-centrifuged at 49500×g. The resulting pellet was re-homogenized and separated by discontinuous sucrose density centrifugation. After ultra-centrifugation at 107000×g, the fractions at the phase boundary were recovered and pelleted.

### 4.1.2 Plasma Membrane Solubilisation

Plasma membrane fractions were resuspended in SDS (Sodium dodecyl sulfate) to give a final SDS concentration of 0.5%, centrifuged and the solubilized protein extracted.

### 4.1.3 Trypsinolysis

For in-solution digestion, the volume of a 50 μg protein solution was made up to 100 μl using 200 mM ammonium bicarbonate. 10 μl of the reducing agent DL-Dithiothreitol (75 mM) was added to the sample and incubated at 80° C. for 15 minutes. This was followed by a cysteine blocking step using 10 μl of 150 mM iodoacetamide and incubation in the dark for 30 minutes at room temperature. The SDS concentration was then diluted to 0.05% with the addition of ultra-pure water. A sufficient volume of trypsin (Promega V5111) was added to the mixture allowing for 1 μg of trypsin to 2.75 μg of protein and incubated overnight at 37° C. Alternatively, 105 μg of protein solutions were reduced using 3 μl of 50 mM TCEP and incubating at 60° C. for 1 hr. The sample was then processed on the FASP filtration devices of the Protein Digestion Kit (Protein Discovery) according to the manufacturer's instructions, but using triethylammonium bicarbonate instead of ammonium bicarbonate. Trypsinolysis was performed in a final volume of 75 μl, using 1 μg of trypsin to 50 μg of protein.

### 4.1.4 Peptide Fractionation

The digested protein samples were dried under a vacuum, re-suspended in 0.1% aqueous formic acid and trifluoroacetic acid (TFA) was added to reduce the pH of the solution to <3. Peptides were separated by ion exchange using an Agilent Zorbax Bio-Strong Cation Exchange series II column on an Agilent LC1200 Series liquid chromatography system. Alternatively, the Agilent 3100 OFFGEL Fractionator and the OFFGEL Kit pH 3-10 was used for pI-based separation, according to the protocol of the supplier. Following re-hydration of the IPG strips, equal volumes of a membrane digest were loaded into each well. Following separation, the resulting fractions were acidified.

### 4.1.5 Mass Spectrometry

Fractionated samples were analysed by liquid chromatography-mass spectrometry using a Waters nanoACQUITY UPLC System fitted with a nanoACQUITY UPLC BEH 130 C18 column, 75 μm×250 mm (186003545) and a LTQ Orbitrap Velos (Thermo Fisher Scientific). Peptides were eluted with a 300 nl/min gradient increasing from 3% to 35% acetonitrile over 120 min. Full-scan mass spectra were acquired at 60000 resolving power between 400-2000 m/z mass range in the Orbitrap. In each cycle, the twenty most intense peptides were selected for CID MS/MS scans in the linear ion trap with nanospray ion source fitted on the instrument.

### 4.1.6 Amino Acid Sequence Analysis of Peptide

The raw data generated from the LTQ Orbitrap Velos was processed through the Mascot software (Matrix Science) which uses the Mowse algorithm (Curr Biol. 1993 Jun. 1; 3(6):327-3) to infer amino acids sequences from the peak lists by searching against a sequence database consisting of Ensembl (worldwide web ensembl.org/index.html), IPI (worldwide web ebi.ac.uk/IPI/IPIhuman.html) and SwissProt (worldwide web uniprot.org) along with contaminant protein sequences. Criteria for peptide identification included trypsin digestion, up to 2 missed cleavage sites and various biological and chemical modifications (oxidized methionine, cysteine modification by MMTS or iodoacetamide and phosphorylation of serine, threonine and tyrosine). Peptides ranked 1 with an expectation value of 0.05% or less, an ion score of 28 or higher were loaded into our OGAP database where they were processed into protein groups.

### 4.1.7 Discrimination of Colorectal Cancer, Gastric Cancer and Pancreatic Cancer Associated Proteins

The process to identify MUC13 (SEQ ID No: 39) used the peptide sequences obtained experimentally by mass spectrometry, as described above, of naturally occurring human proteins to identify and organize coding exons in the published human genome sequence. These experimentally determined sequences indicated in Table 1, were compared with the OGAP® database which was compiled by processing and integration of peptide masses, peptide signatures, ESTs and Public Domain Genomic Sequence Data as described in International Patent Application WO2009/087462.

TABLE 96
MUC13 Specific Peptides Identified By LC/MS in
the plasma membranes of colorectal cancer, gastric
cancer and pancreatic cancer tissue samples.

| SEQ ID No | Peptide Identified |
|---|---|
| SEQ ID No: | CAFGYSGLDCK |
| 1021 | |
| SEQ ID No: | CDYYGCNQTADDCLNGLACDCK |
| 1022 | |
| SEQ ID No: | CPDACNAQHK |
| 1023 | |
| SEQ ID No: | DSQMQNPYSR |
| 895 | |
| SEQ ID No: | DVFGTSVYGQTVILTVSTSLSPR |
| 1024 | |
| SEQ ID No: | HIEEENLIDEDFQNLK |
| 917 | |
| SEQ ID No: | HIEEENLIDEDFQNLKLRSTGFTNLGAEGSVFPK |
| 1025 | |
| SEQ ID No: | HSMAYQDLHSEITSLFK |
| 1026 | |
| SEQ ID No: | HSSMPRPDY |
| 1027 | |
| SEQ ID No: | ISVTVSETFDPEEK |
| 394 | |
| SEQ ID No: | ITASRDSQMQNPYSR |
| 1028 | |
| SEQ ID No: | KSGGAPECACVPGYQEDANGNCQK |
| 1029 | |
| SEQ ID No: | LRSTGFTNLGAEGSVFPK |
| 1030 | |
| SEQ ID No: | SDLQRPNPQSPFCVASSLK |
| 969 | |
| SEQ ID No: | SGGAPECACVPGYQEDANGNCQK |
| 1031 | |
| SEQ ID No: | SSSSNFLNYDLTLR |
| 651 | |
| SEQ ID No: | STGFTNLGAEGSVFPK |
| 974 | |
| SEQ ID No: | TKHIEEENLIDEDFQNLK |
| 1032 | |

### 4.1.8 Protein Index

The protein index is a measure of both protein prevalence and peptide abundance. The algorithm takes into account both the number of samples in which the protein has been observed and the number of peptides observed vs observable peptides from each sample. The resulting value is then graded by pairwise comparison of corresponding normal samples vs cancer samples.

### 4.2 Results

These experiments identified MUC13 as further described herein. The full-length MUC13 was detected in the plasma membrane of colorectal cancer, gastric cancer and pancreatic cancer tissue samples. Table 97 shows the expression distribution of MUC13 measured by the protein index. Expression of MUC13 in these cancer tissues indicates MUC13 is a valuable therapeutic and diagnostic target in these cancers.

TABLE 97
MUC13 Protein Index

| Tissue | Cancer | Normal |
|---|---|---|
| Colorectal | +++++ | ++ |
| Gastric | ++++ | ++ |
| Pancreas | +++ | — |

(+++++ = Very High; ++++ = High; +++ = Medium; ++ = Low; + = Very low; — = Not Observed)

### Example 9

### Immunohistochemistry Using Antibody to MUC13

Using the following Reference Protocol, immunohistochemistry was performed on FFPE tumor and normal tissues using a monoclonal antibody to MUC13.

### 5.1 Materials and Methods

### 5.1.1. Materials

Citroclear (HC5005) from TCS Biosciences, UK.
Reagent alcohol (R8382) from Sigma-Aldrich, UK.
Target Retrieval Solution, pH6 (S2369) from Dako, UK.
REAL Peroxidase Blocking Solution (S2023) from Dako, UK
Antibody Diluent (S0809) from Dako, UK
EnVision+HRP-conjugated polymer, Rabbit (K4002) from Dako, UK.
Liquid DAB+substrate (K3468) from Dako, UK.
Mayer's Hematoxylin (X0909) from Dako, UK
Aquatex (1.08562.0050) from VWR, UK

Tissue sections and arrays were from US Biomax Inc., MD, USA.

### 5.1.2 Deparaffinisation and Rehydration

Slides were deparaffinised in Citroclear (2×5 minutes) then rehydrated through 100% alcohol (2×5 minutes), 50% alcohol (1×5 minutes) and tap water (1×5 minutes).

### 5.1.3 Antigen Retrieval (Pressure Cooker)

The MUC13 antigen was retrieved using microwave heat for 10 minutes in 50 ml Target Retrieval Solution in a Coplin jar. Slides were then left to cool to room temperature for a further 20 min. Circles were drawn around each tissue section/TMA with a hydrophobic barrier pen and slides were then washed twice in PBS, 3 minutes each wash.

### 5.1.4 Tissue Staining

Endogenous peroxidase activity was blocked by incubating tissues with Peroxidase Blocking Solution for 10 minutes at RT in a humidified chamber. Slides were then washed once in PBS and once in PBS-T (PBS containing Tween-20, 0.125% v/v), 3 minutes each wash. Primary antibody (at a concentration of 0.28 ug/ml) was applied to each tissue section and/or microarray, and the slides were incubated for 45 min at room temperature in a humidified chamber. Slides were then washed once in PBS and once in PBS-T, 3 minutes each wash. The EnVision+HRP-conjugated polymer was then applied to the tissues and the slides were incubated for 30 min at room temperature in a humidified chamber. Slides were then washed once in PBS and once in PBS-T, 3 minutes each wash. Tissues were incubated in Liquid DAB+substrate at room temperature for 10 min in a humidified chamber. Slides were then washed once in PBS and once in PBS-T, counterstained with Hematoxylin for 1 min at room temperature in a humidified chamber, and washed again, once in PBS and once in PBS-T, 3 minutes each wash. Coverslips were then mounted onto the slides using Aquatex.

### 5.2 Results

Immunohistochemical analysis revealed specific staining of tumor cells in gastric cancer (Prevalence ca. 75%) and colorectal cancer (Prevalence ca. 50%). At high magnification it was evident that most of the cells were heavily stained in the plasma membrane. Normal stomach staining was observed as well, however at high magnification it was found to be weak cytosolic staining. Thus antibodies directed to MUC13 may have utility as therapeutics and diagnostics in these cancers and other cancer types showing expression of MUC13.

### Example 10

### Specificity of Monoclonal Antibodies to MUC13 Determined by Flow Cytometry Analysis

The specificity of the anti-MUC13 monoclonal antibody was tested by flow cytometry. To test the ability of the antibodies to bind to the cell surface MUC13 protein, the antibodies were incubated with the MUC13-expressing HT-29 and LS174T cells. Cells were washed in FACS buffer (DPBS, 2% FBS), centrifuged and resuspended in 100 μl of the diluted primary MUC13 antibody (also diluted in FACS buffer). The antibody-cell line complex was incubated on ice for 60 min and then washed twice with FACS buffer as described above. The cell-antibody pellet was resuspended in 100 μl of the diluted secondary antibody (also diluted in FACS buffer) and incubated on ice for 60 min on ice. The pellet was washed as before and resuspended in 200 μl FACS buffer. The samples were loaded onto the BD FACScanto II flow sytometer and the data analyzed using the BD FACSdiva software.

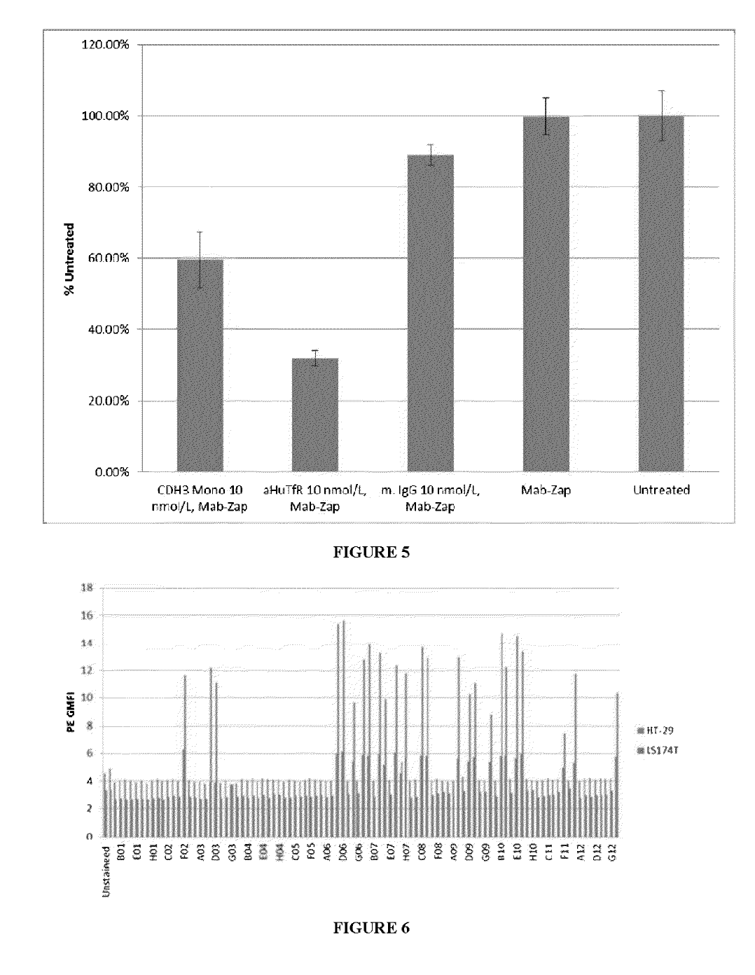

FIG. 6 shows results of flow cytometry analyses, which demonstrated that the anti-MUC13 monoclonal antibodies bound effectively to the cell-surface human MUC13 expressed on HT-29 and LS174T cells.

All references referred to in this application, including patent and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims form part may be used as a basis for priority in respect of any subsequent application. The claims of such subsequent application may be directed to any feature or combination of features described herein. They may take the form of product, composition, process, or use claims and may include, by way of example and without limitation, the following claims:

1. A method for the treatment or prophylaxis of cancer wherein OGTA002, OGTA009, OGTA016, OGTA028, OGTA037, OGTA041, OGTA053, OGTA054, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA222, OGTA236, OGTA237, OGTA247, OGTA248, OGTA249, OGTA257 or OGTA271 is expressed in said cancer, which comprises administering to a subject in need thereof a therapeutically effective amount of an affinity reagent which binds to OGTA002, OGTA009, OGTA016, OGTA028, OGTA037, OGTA041, OGTA053, OGTA054, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA222, OGTA236, OGTA237, OGTA247, OGTA248, OGTA249, OGTA257 or OGTA271.

2. The method according to claim 1, for the treatment or prophylaxis of a cancer selected from the group consisting of B-cell non-Hodgkin's lymphoma, breast cancer, cervical cancer, colorectal cancer, gastric cancer, glioblastoma, hepatocellular carcinoma, lung cancer, lymphoid leukaemia, acute T-cell leukaemia, chronic lymphocytic leukaemia, melanoma, neuroblastoma, osteosarcoma, ovarian cancer, pancreatic cancer, prostate cancer, renal cell cancer and retinoblastoma, or has increased likelihood of developing B-cell non-Hodgkin's lymphoma, breast cancer, cervical cancer, colorectal cancer, gastric cancer, glioblastoma, hepatocellular carcinoma, lung cancer, lymphoid leukaemia, acute T-cell leukaemia, chronic lymphocytic leukaemia, melanoma, neuroblastoma, osteosarcoma, ovarian cancer, pancreatic cancer, prostate cancer, renal cell cancer or retinoblastoma.

3. The method of claim 1, wherein said affinity reagent comprises a label.

4. The method according to claim 3, wherein said label is a detectable label or therapeutic moiety.

5. The method according to claim 4, wherein said therapeutic moiety is selected from the group consisting of a cytotoxic moiety and a radioactive isotype.

6. The method of claim 1, wherein said affinity reagent is selected from the group consisting of fusion proteins and antibodies.

7. The method of claim 6, wherein said antibody is a monoclonal antibody, a humanized antibody, a bispecific antibody, a non-fucosylated antibody, an antibody fragment, or an antibody mimetic.

8. The method of claim 1, wherein said affinity reagent has cytotoxicity against OGTA002, OGTA009, OGTA016, OGTA028, OGTA037, OGTA041, OGTA053, OGTA054, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA222, OGTA236, OGTA237, OGTA247, OGTA248, OGTA249, OGTA257 or OGTA271 antigen expressing cells in the presence of human complement or in the presence of human immune effector cells.

9. A method for screening for or diagnosis of, or for monitoring or assessing treatment of B-cell non-Hodgkin's lymphoma, breast cancer, cervical cancer, colorectal cancer, gastric cancer, glioblastoma, hepatocellular carcinoma, lung cancer, lymphoid leukaemia, acute T-cell leukaemia, chronic lymphocytic leukaemia, melanoma, neuroblastoma, osteosarcoma, ovarian cancer, pancreatic cancer, prostate cancer, renal cell cancer or retinoblastoma in a human subject, which comprises analyzing a sample from a patient for:
a. the presence or absence; or
b. the quantity;
of OGTA002, OGTA009, OGTA016, OGTA028, OGTA037, OGTA041, OGTA053, OGTA054, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA222, OGTA236, OGTA237, OGTA247, OGTA248, OGTA249, OGTA257 or OGTA271, or a fragment thereof, whereby an increase in the level of OGTA002, OGTA009, OGTA016, OGTA028, OGTA037, OGTA041, OGTA053, OGTA054, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA222, OGTA236, OGTA237, OGTA247, OGTA248, OGTA249, OGTA257 or OGTA271, or a fragment thereof relative to a healthy control is indicative of the presence of or increased likelihood of developing B-cell non-Hodgkin's lymphoma, breast cancer, cervical cancer, colorectal cancer, gastric cancer, glioblastoma, hepatocellular carcinoma, lung cancer, lymphoid leukaemia, acute T-cell leukaemia, chronic lymphocytic leukaemia), melanoma, neuroblastoma, osteosarcoma, ovarian cancer, pancreatic cancer, prostate cancer, renal cell cancer or retinoblastoma.

10. The method of claim 9 which comprises contacting said sample with one or more anti-: OGTA002, OGTA009, OGTA016, OGTA028, OGTA037, OGTA041, OGTA053, OGTA054, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA222, OGTA236, OGTA237, OGTA247, OGTA248, OGTA249, OGTA257 or OGTA271 affinity reagents capable of specific binding to OGTA002, OGTA009, OGTA016, OGTA028, OGTA037, OGTA041, OGTA053, OGTA054, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA222, OGTA236, OGTA237, OGTA247, OGTA248, OGTA249, OGTA257 or OGTA271 as defined in SEQ ID Nos: 1-48 respectively or a fragment or derivative thereof.

11. The method according to claim 9, wherein said analyzing comprises imaging said sample.

12. The method of claim 11, wherein said affinity reagent comprises a detectable label.

13. The method of claim 9, wherein said method comprises detecting OGTA002, OGTA009, OGTA016, OGTA028, OGTA037, OGTA041, OGTA053, OGTA054, OGTA072, OGTA074, OGTA076, OGTA085, OGTA087, OGTA088, OGTA089, OGTA091, OGTA098, OGTA101, OGTA104, OGTA106, OGTA112, OGTA113, OGTA119, OGTA124, OGTA126, OGTA156, OGTA159, OGTA168, OGTA169, OGTA174, OGTA176, OGTA177, OGTA197, OGTA202, OGTA203, OGTA206, OGTA213, OGTA214, OGTA222, OGTA236, OGTA237, OGTA247, OGTA248, OGTA249, OGTA257 or OGTA271 mRNA.


SEQUENCE LISTING (1034 sequences)

SEQ ID NO: 1 - protein, 987 aa, Homo Sapiens
  MISC_FEATURE (1)..(987): Designated OGTA002 = Swissprot Accession No. P54760, Ephrin type-B receptor 4
MELRVLLCWASLAAALEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQLALIAGTAVVGVVLVLVVIVVAVLCLRKQSNGREAEYSDKHGQYLIGHGTKVYIDPFTYEDPNEAVREFAKEIDVSYVKIEEVIGAGEFGEVCRGRLKAPGKKESCVAIKTLKGGYTERQRREFLSEASIMGQFEHPNIIRLEGVVTNSMPVMILTEFMENGALDSFLRLNDGQFTVIQLVGMLRGIASGMRYLAEMSYVHRDLAARNILVNSNLVCKVSDFGLSRFLEENSSDPTYTSSLGGKIPIRWTAPEAIAFRKFTSASDAWSYGIVMWEVMSFGERPYWDMSNQDVINAIEQDYRLPPPPDCPTSLHQLMLDCWQKDRNARPRFPQVVSALDKMIRNPASLKIVARENGGASHPLLDQRQPHYSAFGSVGEWLRAIKMGRYEESFAAAGFGSFELVSQISAEDLLRIGVTLAGHQKKILASVQHMKSQAKPGTPGGTGGPAPQY

SEQ ID NO: 2 - protein, 220 aa, Homo Sapiens
  MISC_FEATURE (1)..(220): OGTA009 = Swiss Prot Accession No.  O15551, Claudin-3
MSMGLEITGTALAVLGWLGTIVCCALPMWRVSAFIGSNIITSQNIWEGLWMNCVVQSTGQMQCKVYDSLLALPQDLQAARALIVVAILLAAFGLLVALVGAQCTNCVQDDTAKAKITIVAGVLFLLAALLTLVPVSWSANTIIRDFYNPVVPEAQKREMGAGLYVGWAAAALQLLGGALLCCSCPPREKKYTATKVVYSAPRSTGPGASLGTGYDRKDYV

SEQ ID NO: 3 - protein, 764 aa, Homo Sapiens
  MISC_FEATURE (1)..(764): OGTA016 = Swiss Prot Accession No. P01833, Polymeric-immunoglobulin receptor
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 4 - protein, 865 aa, Homo Sapiens
  MISC_FEATURE (1)..(865): OGTA028 = Swiss Prot Accession No. Q5T7N2, FLJ10884
MSDVSTSVQSKFARLAKKKENITYMKREQLTETDKDIAPVLDLKCKDVSAIMNKFKVLMEIQDLMFEEMRETLKNDLKAVLGGKATIPEVKNSENSSSRTEFQQIINLALQKTGMVGKIEGENSKIGDDNENLTFKLEVNELSGKLDNTNEYNSNDGKKLPQGESRSYEVMGSMEETLCNIDDRDGNRNVHLEFTERESRKDGEDEFVKEMREERKFQKLKNKEEVLKASREEKVLMDEGAVLTLVADLSSATLDISKQWSNVFNILRENDFEPKFLCEVKLAFKCDGEIKTFSDLQSLRKFASQKSSVKELLKDVLPQKEEINQGGRKYGIQEKRDKTLIDSKHRAGEITSDGLSFLFLKEVKVAKPEEMKNLETQEEEFSELEELDEEASGMEDDEDTSGLEEEEEEPSGLEEEEEEEASGLEEDEASGLEEEEEQTSEQDSTFQGHTLVDAKHEVEITSDGMETTFIDSVEDSESEEEEEGKSSETGKVKTTSLTEKKASRRQKEIPFSYLVGDSGKKKLVKHQVVHKTQEEEETAVPTSQGTGTPCLTLCLASPSKSLEMSHDEHKKHSHTNLSISTGVTKLKKTEEKKHRTLHTEELTSKEADLTEETEENLRSSVINSIREIKEEIGNLKSSHSGVLEIENSVDDLSSRMDILEERIDSLEDQIEEFSKDTMQMTKQIISKERQRDIEERSRSCNIRLIGIPEKESYENRAEDIIKEIIDENFAELKKGSSLEIVSACRVPSKIDEKRLTPRHILVKFWNSSDKEKIIRASRERREITYQGTRIRLTADLSLDTLDARSKWSNVFKVLLEKGFNPRILYPAKMAFDFRGKTKVFLSIEEFRDYVLHMPTLRELLGNNIP

SEQ ID NO: 5 - protein, 323 aa, Homo Sapiens
  MISC_FEATURE (1)..(323): OGTA037 = Swiss Prot Accession No. Q9HA72, Protein FAM26B
MAALIAENFRFLSLFFKSKDVMIFNGLVALGTVGSQELFSVVAFHCPCSPARNYLYGLAAIGVPALVLFIIGIILNNHTWNLVAECQHRRTKNCSAAPTFLLLSSILGRAAVAPVTWSVISLLRGEAYVCALSEFVDPSSLTAREEHFPSAHATEILARFPCKENPDNLSDFREEVSRRLRYESQLFGWLLIGVVAILVFLTKCLKHYCSPLSYRQEAYWAQYRANEDQLFQRTAEVHSRVLAANNVRRFFGFVALNKDDEELIANFPVEGTQPRPQWNAITGVYLYRENQGLPLYSRLHKWAQGLAGNGAAPDNVEMALLPS

SEQ ID NO: 6 - protein, 918 aa, Homo Sapiens [sequence omitted]

SEQ ID NO: 7 - protein, 1042 aa, Homo Sapiens
  MISC_FEATURE (1)..(1042): OGTA053 = Swiss Prot Accession No. Q9Y4D2, Sn1-specific diacylglycerol lipase alpha
MPGIVVFRRRWSVGSDDLVLPAIFLFLLHTTWFVILSVVLFGLVYNPHEACSLNLVDHGRGYLGILLSCMIAEMAIIWLSMRGGILYTEPRDSMQYVLYVRLAILVIEFIYAIVGIVWLTQYYTSCNDLTAKNVTLGMVVCNWVVILSVCITVLCVFDPTGRTFVKLRATKRRQRNLRTYNLRHRLEEGQATSWSRRLKVFLCCTRTKDSQSDAYSEIAYLFAEFFRDLDIVPSDIIAGLVLLRQRQRAKRNAVLDEANNDILAFLSGMPVTRNTKYLDLKNSQEMLRYKEVCYYMLFALAAYGWPMYLMRKPACGLCQLARSCSCCLCPARPRFAPGVTIEEDNCCGCNAIAIRRHFLDENMTAVDIVYTSCHDAVYETPFYVAVDHDKKKVVISIRGTLSPKDALTDLTGDAERLPVEGHHGTWLGHKGMVLSAEYIKKKLEQEMVLSQAFGRDLGRGTKHYGLIVVGHSLGAGTAAILSFLLRPQYPTLKCFAYSPPGGLLSEDAMEYSKEFVTAVVLGKDLVPRIGLSQLEGFRRQLLDVLQRSTKPKWRIIVGATKCIPKSELPEEVEVTTLASTRLWTHPSDLTIALSASTPLYPPGRIIHVVHNHPAEQCCCCEQEEPTYFAIWGDNKAFNEVIISPAMLHEHLPYVVMEGLNKVLENYNKGKTALLSAAKVMVSPTEVDLTPELIFQQQPLPTGPPMPTGLALELPTADHRNSSVRSKSQSEMSLEGFSEGRLLSPVVAAAARQDPVELLLLSTQERLAAELQARRAPLATMESLSDTESLYSFDSRRSSGFRSIRGSPSLHAVLERDEGHLFYIDPAIPEENPSLSSRTELLAADSLSKHSQDTQPLEAALGSGGVTPERPPSAAANDEEEEVGGGGGGPASRGELALHNGRLGDSPSPQVLEFAEFIDSLFNLDSKSSSFQDLYCMVVPESPTSDYAEGPKSPSQQEILLRAQFEPNLVPKPPRLFAGSADPSSGISLSPSFPLSSSGELMDLTPTGLSSQECLAADKIRTSTPTGHGASPAKQDELVISAR

SEQ ID NO: 8 - protein, 125 aa, Homo Sapiens
  MISC_FEATURE (1)..(125): OGTA054 = Swiss Prot Accession No. P13164, Interferon-induced transmembrane protein 1
MHKEEHEVAVLGAPPSTILPRSTVINIHSETSVPDHVVWSLFNTLFLNWCCLGFIAFAYSVKSRDRKMVGDVTGAQAYASTAKCLNIWALILGILMTIGFILLLVFGSVTVYHIMLQIIQEKRGY

SEQ ID NO: 9 - protein, 166 aa, Homo Sapiens
  MISC_FEATURE (1)..(166): OGTA066 = Swiss Prot Accession No. Q8TD06, Anterior gradient protein 3 homolog
MMLHSALGLCLLLVTVSSNLAIAIKKEKRPPQTLSRGWGDDITWVQTYEEGLFYAQKSKKPLMVIHHLEDCQYSQALKKVFAQNEEIQEMAQNKFIMLNLMHETTDKNLSPDGQYVPRIMFVDPSLTVRADIAGRYSNRLYTYEPRDLPLLIENMKKALRLIQSEL

SEQ ID NO: 10 - protein, 407 aa, Homo Sapiens
  MISC_FEATURE (1)..(407): OGTA072 = Swiss Prot Accession No. Q16186, Adhesion-regulating molecule 1
MTTSGALFPSLVPGSRGASNKYLVEFRAGKMSLKGTTVTPDKRKGLVYIQQTDDSLIHFCWKDRTSGNVEDDLIIFPDDCEFKRVPQCPSGRVYVLKFKAGSKRLFFWMQEPKTDQDEEHCRKVNEYLNNPPMPGALGASGSSGHELSALGGEGGLQSLLGNMSHSQLMQLIGPAGLGGLGGLGALTGPGLASLLGSSGPPGSSSSSSSRSQSAAVTPSSTTSSTRATPAPSAPAAASATSPSPAPSSGNGASTAASPTQPIQLSDLQSILATMNVPAGPAGGQQVDLASVLTPEIMAPILANADVQERLLPYLPSGESLPQTADEIQNTLTSPQFQQALGMFSAALASGQLGPLMCQFGLPAEAVEAANKGDVEAFAKAMQNNAKPEQKEGDTKDKKDEEEDMSLD

SEQ ID NO: 11 - protein, 1587 aa, Homo Sapiens
  MISC_FEATURE (1)..(1587): OGTA074 = Swiss Prot Accession No. O00508, Latent TGF-beta binding protein-4
MGDVKALLFVAAARARRLGGAAASESLAVSEAFCRVRSCQPKKCAGPQRCLNPVPAVPSPSPSVRKRQVSLNWQPLTLQEARALLKRRRPRGPGGRGLLRRRPPQRAPAGKAPVLCPLICHNGGVCVKPDRCLCPPDFAGKFCQLHSSGARPPAPAVPGLTRSVYTMPLANHRDDEHGVASMVSVHVEHPQEASVVVHQVERVSGPWEEADAEAVARAEAAARAEAAAPYTVLAQSAPREDGYSDASGFGYCFRELRGGECASPLPGLRTQEVCCRGAGLAWGVHDCQLCSERLGNSERVSAPDGPCPTGFERVNGSCEDVDECATGGRCQHGECANTRGGYTCVCPDGFLLDSSRSSCISQHVISEAKGPCFRVLRDGGCSLPILRNITKQICCCSRVGKAWGRGCQLCPPFGSEGFREICPAGPGYHYSASDLRYNTRPLGQEPPRVSLSQPRTLPATSRPSAGFLPTHRLEPRPEPRPDPRPGPELPLPSIPAWTGPEIPESGPSSGMCQRNPQVCGPGRCISRPSGYTCACDSGFRLSPQGTRCIDVDECRRVPPPCAPGRCENSPGSFRCVCGPGFRAGPRAAECLDVDECHRVPPPCDLGRCENTPGSFLCVCPAGYQAAPHGASCQDVDECTQSPGLCGRGACKNLPGSFRCVCPAGFRGSACEEDVDECAQEPPPCGPGRCDNTAGSFHCACPAGFRSRGPGAPCQDVDECARSPPPCTYGRCENTEGSFQCVCPMGFQPNTAGSECEDVDECENHLACPGQECVNSPGSFQCRTCPSGHHLHRGRCTDVDECSSGAPPCGPHGHCTNTEGSFRCSCAPGYRAPSGRPGPCADVNECLEGDFCFPHGECLNTDGSFACTCAPGYRPGPRGASCLDVDECSEEDLCQSGICTNTDGSFECICPPGHRAGPDLASCLDVDECRERGPALCGSQRCENSPGSYRCVRDCDPGYHAGPEGTCDDVDECQEYGPEICGAQRCENTPGSYRCTPACDPGYQPTPGGGCQDVDECRNRSFCGAHAVCQNLPGSFQCLCDQGYEGARDGRHCVDVNECETLQGVCGAALCENVEGSFLCVCPNSPEEFDPMTGRCVPPRTSAGTFPGSQPQAPASPVLPARPPPPPLPRRPSTPRQGPVGSGRRECYFDTAAPDACDNILARNVTWQECCCTVGEGWGSGCRIQQCPGTETAEYQSLCPHGRGYLAPSGDLSLRRDVDECQLFRDQVCKSGVCVNTAPGYSCYCSNGYYYHTQRLECIDNDECADEEPACEGGRCVNTVGSYHCTCEPPLVLDGSQRRCVSNESQSLDDNLGVCWQEVGADLVCSHPRLDRQATYTECCCLYGEAWGMDCALCPAQDSDDFEALCNVLRPPAYSPPRPGGFGLPYEYGPDLGPPYQGLPYGPELYPPPALPYDPYPPPPGPFARREAPYGAPRFDMPDFEDDGGPYGESEAPAPPGPGTRWPYRSRDTRRSFPEPEEPPEGGSYAGSLAEPYEELEAEECGILDGCTNGRCVRVPEGFTCRCFDGYRLDMTRMACVDINECDEAEAASPLCVNARCLNTDGSFRCICRPGFAPTHQPHHCAPARPRA

SEQ ID NO: 12 - protein, 1722 aa, Homo Sapiens
  MISC_FEATURE (1)..(1722): OGTA076 = Swiss Prot Accession No. O60449, Lymphocyte antigen 75
MRTGWATPRRPAGLLMLLFWFFDLAEPSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICLKPENGCEDNWEKNEQFGSCYQFNTQTALSWKEAYVSCQNQGADLLSINSAAELTYLKEKEGIAKIFWIGLNQLYSARGWEWSDHKPLNFLNWDPDRPSAPTIGGSSCARMDAESGLWQSFSCEAQLPYVCRKPLNNTVELTDVWTYSDTRCDAGWLPNNGFCYLLVNESNSWDKAHAKCKAFSSDLISIHSLADVEVVVTKLHNEDIKEEVWIGLKNINIPTLFQWSDGTEVTLTYWDENEPNVPYNKTPNCVSYLGELGQWKVQSCEEKLKYVCKRKGEKLNDASSDKMCPPDEGWKRHGETCYKIYEDEVPFGTNCNLTITSRFEQEYLNDLMKKYDKSLRKYFWTGLRDVDSCGEYNWATVGGRRRAVTFSNWNFLEPASPGGCVAMSTGKSVGKWEVKDCRSFKALSICKKMSGPLGPEEASPKPDDPCPEGWQSFPASLSCYKVFHAERIVRKRNWEEAERFCQALGAHLSSFSHVDEIKEFLHFLTDQFSGQHWLWIGLNKRSPDLQGSWQWSDRTPVSTIIMPNEFQQDYDIRDCAAVKVFHRPWRRGWHFYDDREFIYLRPFACDTKLEWVCQIPKGRTPKTPDWYNPDRAGIHGPPLIIEGSEYWFVADLHLNYEEAVLYCASNHSFLATITSFVGLKAIKNKIANISGDGQKWWIRISEWPIDDHFTYSRYPWHRFPVTFGEECLYMSAKTWLIDLGKPTDCSTKLPFICEKYNVSSLEKYSPDSAAKVQCSEQWIPFQNKCFLKIKPVSLTFSQASDTCHSYGGTLPSVLSQIEQDFITSLLPDMEATLWIGLRWTAYEKINKWTDNRELTYSNFHPLLVSGRLRIPENFFEEESRYHCALILNLQKSPFTGTWNFTSCSERHFVSLCQKYSEVKSRQTLQNASETVKYLNNLYKIIPKTLTWHSAKRECLKSNMQLVSITDPYQQAFLSVQALLHNSSLWIGLFSQDDELNFGWSDGKRLHFSRWAETNGQLEDCVVLDTDGFWKTVDCNDNQPGAICYYSGNETEKEVKPVDSVKCPSPVLNTPWIPFQNCCYNFIITKNRHMATTQDEVHTKCQKLNPKSHILSIRDEKENNFVLEQLLYFNYMASWVMLGITYRNNSLMWFDKTPLSYTHWRAGRPTIKNEKFLAGLSTDGFWDIQTFKVIEEAVYFHQHSILACKIEMVDYKEEHNTTLPQFMPYEDGIYSVIQKKVTWYEALNMCSQSGGHLASVHNQNGQLFLEDIVKRDGFPLWVGLSSHDGSESSFEWSDGSTFDYIPWKGQTSPGNCVLLDPKGTWKHEKCNSVKDGAICYKPTKSKKLSRLTYSSRCPAAKENGSRWIQYKGHCYKSDQALHSFSEAKKLCSKHDHSATIVSIKDEDENKFVSRLMRENNNITMRVWLGLSQHSVDQSWSWLDGSEVTFVKWENKSKSGVGRCSMLIASNETWKKVECEHGFGRVVCKVPLGPDYTAIAIIVATLSILVLMGGLIWFLFQRHRLHLAGFSSVRYAQGVNEDEIMLPSFHD

SEQ ID NO: 13 - protein, 355 aa, Homo Sapiens
  MISC_FEATURE (1)..(355): OGTA085 = Swiss Prot Accession No. Q14318, 38 kDa FK506-binding protein homologue
MGQPPAEEAEQPGALAREFLAAMEPEPAPAPAPEEWLDILGNGLLRKKTLVPGPPGSSRPVKGQVVTVHLQTSLENGTRVQEEPELVFTLGDCDVIQALDLSVPLMDVGETAMVTADSKYCYGPQGRSPYIPPHAALCLEVTLKTAVDGPDLEMLTGQERVALANRKRECGNAHYQRADFVLAANSYDLAIKAITSSAKVDMTFEEEAQLLQLKVKCLNNLAASQLKLDHYRAALRSCSLVLEHQPDNIKALFRKGKVLAQQGEYSEAIPILRAALKLEPSNKTIHAELSKLVKKHAAQRSTETALYRKMLGNPSRLPAKCPGKGAWSIPWKWLFGATAVALGGVALSVVIAARN

SEQ ID NO: 14 - protein, 587 aa, Homo Sapiens
  MISC_FEATURE (1)..(587): OGTA087 = Swiss Prot Accession No. O94935, KIAA0851 protein
MATAAYEQLKLHITPEKFYVEACDDGADDVLTIDRVSTEVTLAVKKDVPPSAVTRPIFGILGTIHLVAGNYLIVITKKIKVGEFFSHVVWKATDFDVLSYKKTMLHLTDIQLQDNKTFLAMLNHVLNVDGFYFSTTYDLTHTLQRLSNTSPEFQEMSLLERADQRFVWNGHLLRELSAQPEVHRFALPVLHGFITMHSCSINGKYFDWILISRRSCFRAGVRYYVRGIDSEGHAANFVETEQIVHYNGSKASFVQTRGSIPVFWSQRPNLKYKPLPQISKVANHMDGFQRHFDSQVIIYGKQVIINLINQKGSEKPLEQTFATMVSSLGSGMMRYIAFDFHKECKNMRWDRLSILLDQVAEMQDELSYFLVDSAGQVVANQEGVFRSNCMDCLDRTNVIQSLLARRSLQAQLQRLGVLHVGQKLEEQDEFEKIYKNAWADNANACAKQYAGTGALKTDFTRTGKRTHLGLIMDGWNSMIRYYKNNFSDGFRQDSIDLFLGNYSVDELESHSPLSVPRDWKFLALPIIMVVAFSMCIICLLMAGDTWTETLAYVLFWGVASIGTFFIILYNGKDFVDAPRLVQKEKID

SEQ ID NO: 15 - protein, 338 aa, Homo Sapiens
  MISC_FEATURE (1)..(338): OGTA088 = Swiss Prot Accession No. O15126, Secretory carrier-associated membrane protein 1
MSDFDSNPFADPDLNNPFKDPSVTQVTRNVPPGLDEYNPFSDSRTPPPGGVKMPNVPNTQPAIMKPTEEHPAYTQIAKEHALAQAELLKRQEELERKAAELDRREREMQNLSQHGRKNNWPPLPSNFPVGPCFYQDFSVDIPVEFQKTVKLMYYLWMFHAVTLFLNIFGCLAWFCVDSARAVDFGLSILWFLLFTPCSFVCWYRPLYGAFRSDSSFRFFVFFFVYICQFAVHVLQAAGFHNWGNCGWISSLTGLNQNIPVGIMMIIIAALFTASAVISLVMFKKVHGLYRTTGASFEKAQQEFATGVMSNKTVQTAAANAASTAASSAAQNAFKGNQI

SEQ ID NO: 16 - protein, 1058 aa, Homo Sapiens
  MISC_FEATURE (1)..(1058): OGTA089 = Swiss Prot Accession No. Q5T225, Polycystic kidney disease 1-like
MEKRLGVKPNPASWILSGYYWQTSAKWLRSLYLFYTCFCFSVLWLSTDASESRCQQGKTQFGVGLRSGGENHLWLLEGTPSLQSCWAACCQDSACHVFWWLEGMCIQADCSRPQSCRAFRTHSSNSMLVFLKKFQTADDLGFLPEDDVPHLLGLGWNWASWRQSPPRAALRPAVSSSDQQSLIRKLQKRGSPSDVVTPIVTQHSKVNDSNELGGLTTSGSAEVHKAITISSPLTTDLTAELSGGPKNVSVQPEISEGLATTPSTQQVKSSEKTQIAVPQPVAPSYSYATPTPQASFQSTSAPYPVIKELVVSAGESVQITLPKNEVQLNAYVLQEPPKGETYTYDWQLITHPRDYSGEMEGKHSQILKLSKLTPGLYEFKVIVEGQNAHGEGYVNVTVKPEPRKNRPPIAIVSPQFQEISLPTTSTVIDGSQSTDDDKIVQYHWEELKGPLREEKISEDTAILKLSKLVPGNYTFSLTVVDSDGATNSTTANLTVNKAVDYPPVANAGPNQVITLPQNSITLFGNQSTDDHGITSYEWSLSPSSKGKVVEMQGVRTPTLQLSAMQEGDYTYQLTVTDTIGQQATAQVTVIVQPENNKPPQADAGPDKELTLPVDSTTLDGSKSSDDQKIISYLWEKTQGPDGVQLENANSSVATVTGLQVGTYVFTLTVKDERNLQSQSSVNVIVKEEINKPPIAKITGNVVITLPTSTAELDGSKSSDDKGIVSYLWTRDEGSPAAGEVLNHSDHHPILFLSNLVEGTYTFHLKVTDAKGESDTDRTTVEVKPDPRKNNLVEIILDINVSQLTERLKGMFIRQIGVLLGVLDSDIIVQKIQPYTEQSTKMVFFVQNEPPHQIFKGHEVAAMLKSELRKQKADFLIFRALEVNTVTCQLNCSDHGHCDSFTKRCICDPFWMENFIKVQLRDGDSNCALTILIFLAEWSVLYVIIATFVIVVALGILSWTVICCCKRQKGKPKRKSKYKILDATDQESLELKPTSRAGIKQKGLLLSSSLMHSESELDSDDAIFTWPDREKGKLLHGQNGSVPNGQTPLKARSPREEIL

SEQ ID NO: 17 - protein, 2080 aa, Homo Sapiens
  MISC_FEATURE (1)..(2080): OGTA091 = Swiss Prot Accession No. O75923, Dysferlin
MLRVFILYAENVHTPDTDISDAYCSAVFAGVKKRTKVIKNSVNPVWNEGFEWDLKGIPLDQGSELHVVVKDHETMGRNRFLGEAKVPLREVLATPSLSASFNAPLLDTKKQPTGASLVLQVSYTPLPGAVPLFPPPTPLEPSPTLPDLDVVADTGGEEDTEDQGLTGDEAEPFLDQSGGPGAPTTPRKLPSRPPPHYPGIKRKRSAPTSRKLLSDKPQDFQIRVQVIEGRQLPGVNIKPVVKVTAAGQTKRTRIHKGNSPLFNETLFFNLFDSPGELFDEPIFITVVDSRSLRTDALLGEFRMDVGTIYREPRHAYLRKWLLLSDPDDFSAGARGYLKTSLCVLGPGDEAPLERKDPSEDKEDIESNLLRPTGVALRGAHFCLKVFRAEDLPQMDDAVMDNVKQIFGFESNKKNLVDPFVEVSFAGKMLCSKILEKTANPQWNQNITLPAMFPSMCEKMRIRIIDWDRLTHNDIVATTYLSMSKISAPGGEIEEEPAGAVKPSKASDLDDYLGFLPTFGPCYINLYGSPREFTGFPDPYTELNTGKGEGVAYRGRLLLSLETKLVEHSEQKVEDLPADDILRVEKYLRRRKYSLFAAFYSATMLQDVDDAIQFEVSIGNYGNKFDMTCLPLASTTQYSRAVFDGCHYYYLPWGNVKPVVVLSSYWEDISHRIETQNQLLGIADRLEAGLEQVHLALKAQCSTEDVDSLVAQLTDELIAGCSQPLGDIHETPSATHLDQYLYQLRTHHLSQITEAALALKLGHSELPAALEQAEDWLLRLRALAEEPQNSLPDIVIWMLQGDKRVAYQRVPAHQVLFSRRGANYCGKNCGKLQTIFLKYPMEKVPGARMPVQIRVKLWFGLSVDEKEFNQFAEGKLSVFAETYENETKLALVGNWGTTGLTYPKFSDVTGKIKLPKDSFRPSAGWTWAGDWFVCPEKTLLHDMDAGHLSFVEEVFENQTRLPGGQWIYMSDNYTDVNGEKVLPKDDIECPLGWKWEDEEWSTDLNRAVDEQGWEYSITIPPERKPKHWVPAEKMYYTHRRRRWVRLRRRDLSQMEALKRHRQAEAEGEGWEYASLFGWKFHLEYRKTDAFRRRRWRRRMEPLEKTGPAAVFALEGALGGVMDDKSEDSMSVSTLSFGVNRPTISCIFDYGNRYHLRCYMYQARDLAAMDKDSFSDPYAIVSFLHQSQKTVVVKNTLNPTWDQTLIFYEIEIFGEPATVAEQPPSIVVELYDHDTYGADEFMGRCICQPSLERMPRLAWFPLTRGSQPSGELLASFELIQREKPAIHHIPGFEVQETSRILDESEDTDLPYPPPQREANIYMVPQNIKPALQRTAIEILAWGLRNMKSYQLANISSPSLVVECGGQTVQSCVIRNLRKNPNFDICTLFMEVMLPREELYCPPITVKVIDNRQFGRRPVVGQCTIRSLESFLCDPYSAESPSPQGGPDDVSLLSPGEDVLIDIDDKEPLIPIQEEEFIDWWSKFFASIGEREKCGSYLEKDFDTLKVYDTQLENVEAFEGLSDFCNTFKLYRGKTQEETEDPSVIGEFKGLFKIYPLPEDPAIPMPPRQFHQLAAQGPQECLVRIYIVRAFGLQPKDPNGKCDPYIKISIGKKSVSDQDNYIPCTLEPVFGKMFELTCTLPLEKDLKITLYDYDLLSKDEKIGETVVDLENRLLSKFGARCGLPQTYCVSGPNQWRDQLRPSQLLHLFCQQHRVKAPVYRTDRVMFQDKEYSIEEIEAGRIPNPHLGPVEERLALHVLQQQGLVPEHVESRPLYSPLQPDIEQGKLQMWVDLFPKALGRPGPPFNITPRRARRFFLRCIIWNTRDVILDDLSLTGEKMSDIYVKGWMIGFEEHKQKTDVHYRSLGGEGNFNWRFIFPFDYLPAEQVCTIAKKDAFWRLDKTESKIPARVVFQIWDNDKFSFDDFLGSLQLDLNRMPKPAKTAKKCSLDQLDDAFHPEWFVSLFEQKTVKGWWPCVAEEGEKKILAGKLEMTLEIVAESEHEERPAGQGRDEPNMNPKLEDPRRPDTSFLWFTSPYKTMKFILWRRFRWAIILFIILFILLLFLAIFIYAFPNYAAMKLVKPFS

SEQ ID NO: 18 - protein, 1117 aa, Homo Sapiens
  MISC_FEATURE (1)..(1117): OGTA098 = Swiss Prot Accession No. Q14126, Desmoglein-2
MARTRDRVRLLLLLICFNVGSGLHLQVLSTRNENKLLPKHPHLVRQKRAWITAPVALREGEDLSKKNPIAKIHSDLAEERGLKITYKYTGKGITEPPFGIFVFNKDTGELNVTSILDREETPFFLLTGYALDARGNNVEKPLELRIKVLDINDNEPVFTQDVFVGSVEELSAAHTLVMKINATDADEPNTLNSKISYRIVSLEPAYPPVFYLNKDTGEIYTTSVTLDREEHSSYTLTVEARDGNGEVTDKPVKQAQVQIRILDVNDNIPVVENKVLEGMVEENQVNVEVTRIKVFDADEIGSDNWLANFTFASGNEGGYFHIETDAQTNEGIVTLIKEVDYEEMKNLDFSVIVANKAAFHKSIRSKYKPTPIPIKVKVKNVKEGIHFKSSVISIYVSESMDRSSKGQIIGNFQAFDEDTGLPAHARYVKLEDRDNWISVDSVTSEIKLAKLPDFESRYVQNGTYTVKIVAISEDYPRKTITGTVLINVEDINDNCPTLIEPVQTICHDAEYVNVTAEDLDGHPNSGPFSFSVIDKPPGMAEKWKIARQESTSVLLQQSEKKLGRSEIQFLISDNQGFSCPEKQVLTLTVCEVLHGSGCREAQHDSYVGLGPAAIALMILAFLLLLLVPLLLLMCHCGKGAKAFTPIPGTIEMLHPWNNEGAPPEDKVVPSFLPVDQGGSLVGRNGVGGMAKEATMKGSSSASIVKGQHEMSEMDGRWEEHRSLLSGRATQFTGATGAIMTTETTKTARATGASRDMAGAQAAAVALNEEFLRNYFTDKAASYTEEDENHTAKDCLLVYSQEETESLNASIGCCSFIEGELDDRFLDDLGLKFKTLAEVCLGQKIDINKEIEQRQKPATETSMNTASHSLCEQTMVNSENTYSSGSSFPVPKSLQEANAEKVTQEIVTERSVSSRQAQKVATPLPDPMASRNVIATETSYVTGSTMPPTTVILGPSQPQSLIVTERVYAPASTLVDQPYANEGTVVVTERVIQPHGGGSNPLEGTQHLQDVPYVMVRERESFLAPSSGVQPTLAMPNIAVGQNVTVTERVLAPASTLQSSYQIPTENSMTARNTTVSGAGVPGPLPDFGLEESGHSNSTITTSSTRVTKHSTVQHSYS

SEQ ID NO: 19 - protein, 1912 aa, Homo Sapiens
  MISC_FEATURE (1)..(1912): OGTA101 = Swiss Prot Accession No. P23468, Receptor-type tyrosine-protein phosphatase delta
MVHVARLLLLLLTFFLRTDAETPPRFTRTPVDQTGVSGGVASFICQATGDPRPKIVWNKKGKKVSNQRFEVIEFDDGSGSVLRIQPLRTPRDEAIYECVASNNVGEISVSTRLTVLREDQIPRGFPTIDMGPQLKVVERTRTATMLCAASGNPDPEITWFKDFLPVDTSNNNGRIKQLRSESIGGTPIRGALQIEQSEESDQGKYECVATNSAGTRYSAPANLYVRELREVRRVPPRFSIPPTNHEIMPGGSVNITCVAVGSPMPYVKWMLGAEDLTPEDDMPIGRNVLELNDVRQSANYTCVAMSTLGVIEAIAQITVKALPKPPGTPVVTESTATSITLTWDSGNPEPVSYYIIQHKPKNSEELYKEIDGVATTRYSVAGLSPYSDYEFRVVAVNNIGRGPPSEPVLTQTSEQAPSSAPRDVQARMLSSTTILVQWKEPEEPNGQIQGYRVYYTMDPTQHVNNWMKHNVADSQITTIGNLVPQKTYSVKVLAFTSIGDGPLSSDIQVITQTGVPGQPLNFKAEPESETSILLSWTPPRSDTIANYELVYKDGEHGEEQRITIEPGTSYRLQGLKPNSLYYFRLAARSPQGLGASTAEISARTMQSKPSAPPQDISCTSPSSTSILVSWQPPPVEKQNGIITEYSIKYTAVDGEDDKPHEILGIPSDTTKYLLEQLEKWTEYRITVTAHTDVGPGPESLSVLIRTNEDVPSGPPRKVEVEAVNSTSVKVSWRSPVPNKQHGQIRGYQVHYVRMENGEPKGQPMLKDVMLADAQWEFDDTTEHDMIISGLQPETSYSLTVTAYTTKGDGARSKPKLVSTTGAVPGKPRLVINHTQMNTALIQWHPPVDTFGPLQGYRLKFGRKDMEPLTTLEFSEKEDHFTATDIHKGASYVFRLSARNKVGFGEEMVKEISIPEEVPTGFPQNLHSEGTTSTSVQLSWQPPVLAERNGIITKYTLLYRDINIPLLPMEQLIVPADTTMTLTGLKPDTTYDVKVRAHTSKGPGPYSPSVQFRTLPVDQVFAKNFHVKAVMKTSVLLSWEIPENYNSAMPFKILYDDGKMVEEVDGRATQKLIVNLKPEKSYSFVLTNRGNSAGGLQHRVTAKTAPDVLRTKPAFIGKTNLDGMITVQLPEVPANENIKGYYIIIVPLKKSRGKFIKPWESPDEMELDELLKEISRKRRSIRYGREVELKPYIAAHFDVLPTEFTLGDDKHYGGFTNKQLQSGQEYVFFVLAVMEHAESKMYATSPYSDPVVSMDLDPQPITDEEEGLIWVVGPVLAVVFIICIVIAILLYKRKRAESDSRKSSIPNNKEIPSHHPTDPVELRRLNFQTPGMASHPPIPILELADHIERLKANDNLKFSQEYESIDPGQQFTWEHSNLEVNKPKNRYANVIAYDHSRVLLSAIEGIPGSDYVNANYIDGYRKQNAYIATQGSLPETFGDFWRMIWEQRSATVVMMTKLEERSRVKCDQYWPSRGTETHGLVQVTLLDTVELATYCVRTFALYKNGSSEKREVRQFQFTAWPDHGVPEHPTPFLAFLRRVKTCNPPDAGPMVVHCSAGVGRTGCFIVIDAMLERIKHEKTVDIYGHVTLMRAQRNYMVQTEDQYIFIHDALLEAVTCGNTEVPARNLYAYIQKLTQIETGENVTGMELEFKRLASSKAHTSRFISANLPCNKFKNRLVNIMPYESTRVCLQPIRGVEGSDYINASFIDGYRQQKAYIATQGPLAETTEDFWRMLWEHNSTIVVMLTKLREMGREKCHQYWPAERSARYQYFVVDPMAEYNMPQYILREFKVTDARDGQSRTVRQFQFTDWPEQGVPKSGEGFIDFIGQVHKTKEQFGQDGPISVHCSAGVGRTGVFITLSIVLERMRYEGVVDIFQTVKMLRTQRPAMVQTEDQYQFSYRAALEYLGSFDHYAT

SEQ ID NO: 20 - protein, 819 aa, Homo Sapiens
  MISC_FEATURE (1)..(819): OGTA104 = Swiss Prot Accession No. Q13443, ADAM 9
MGSGARFPSGTLRVRWLLLLGLVGPVLGAARPGFQQTSHLSSYEIITPWRLTRERREAPRPYSKQVSYVIQAEGKEHIIHLERNKDLLPEDFVVYTYNKEGTLITDHPNIQNHCHYRGYVEGVHNSSIALSDCFGLRGLLHLENASYGIEPLQNSSHFEHIIYRMDDVYKEPLKCGVSNKDIEKETAKDEEEEPPSMTQLLRRRRAVLPQTRYVELFIVVDKERYDMMGRNQTAVREEMILLANYLDSMYIMLNIRIVLVGLEIWTNGNLINIVGGAGDVLGNFVQWREKFLITRRRHDSAQLVLKKGFGGTAGMAFVGTVCSRSHAGGINVFGQITVETFASIVAHELGHNLGMNHDDGRDCSCGAKSCIMNSGASGSRNFSSCSAEDFEKLTLNKGGNCLLNIPKPDEAYSAPSCGNKLVDAGEECDCGTPKECELDPCCEGSTCKLKSFAECAYGDCCKDCRFLPGGTLCRGKTSECDVPEYCNGSSQFCQPDVFIQNGYPCQNNKAYCYNGMCQYYDAQCQVIFGSKAKAAPKDCFIEVNSKGDRFGNCGFSGNEYKKCATGNALCGKLQCENVQEIPVFGIVPAIIQTPSRGTKCWGVDFQLGSDVPDPGMVNEGTKCGAGKICRNFQCVDASVLNYDCDVQKKCHGHGVCNSNKNCHCENGWAPPNCETKGYGGSVDSGPTYNEMNTALRDGLLVFFFLIVPLIVCAIFIFIKRDQLWRSYFRKKRSQTYESDGKNQANPSRQPGSVPRHVSPVTPPREVPIYANRFAVPTYAAKQPQQFPSRPPPPQPKVSSQGNLIPARPAPAPPLYSSLT

SEQ ID NO: 21 - protein, 2003 aa, Homo Sapiens
  MISC_FEATURE (1)..(2003): OGTA106 = Swiss Prot Accession No. Q99466, Neurogenic locus notch homologue protein 4
MQPPSLLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPDCEVNPDNCVSHQCQNGGTCQDGLDTYTCLCPETWTGWDCSEDVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEVGQAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 22 - protein, 390 aa, Homo Sapiens
  MISC_FEATURE (1)..(390): OGTA112 = Swiss Prot Accession No. P48594, Serpin B4
MNSLSEANTKFMFDLFQQFRKSKENNIFYSPISITSALGMVLLGAKDNTAQQISKVLHFDQVTENTTEKAATYHVDRSGNVHHQFQKLLTEFNKSTDAYELKIANKLFGEKTYQFLQEYLDAIKKFYQTSVESTDFANAPEESRKKINSWVESQTNEKIKNLFPDGTIGNDTTLVLVNAIYFKGQWENKFKKENTKEEKFWPNKNTYKSVQMMRQYNSFNFALLEDVQAKVLEIPYKGKDLSMIVLLPNEIDGLQKLEEKLTAEKLMEWTSLQNMRETCVDLHLPRFKMEESYDLKDTLRTMGMVNIFNGDADLSGMTWSHGLSVSKVLHKAFVEVTEEGVEAAAATAVVVVELSSPSTNEEFCCNHPFLFFIRQNKTNSILFYGRFSSP

SEQ ID NO: 23 - protein, 400 aa, Homo Sapiens
  MISC_FEATURE (1)..(400): OGTA113 = Swiss Prot Accession No. Q9BTV4, Transmembrane protein 43
MAANYSSTSTRREHVKVKTSSQPGFLERLSETSGGMFVGLMAFLLSFYLIFTNEGRALKTATSLAEGLSLVVSPDSIHSVAPENEGRLVHIIGALRTSKLLSDPNYGVHLPAVKLRRHVEMYQWVETEESREYTEDGQVKKETRYSYNTEWRSEIINSKNFDREIGHKNPSAMAVESFMATAPFVQIGRFFLSSGLIDKVDNFKSLSLSKLEDPHVDIIRRGDFFYHSENPKYPEVGDLRVSFSYAGLSGDDPDLGPAHVVTVIARQRGDQLVPFSTKSGDTLLLLHHGDFSAEEVFHRELRSNSMKTWGLRAAGWMAMFMGLNLMTRILYTLVDWFPVFRDLVNIGLKAFAFCVATSLTLLTVAAGWLFYRPLWALLIAGLALVPILVARTRVPAKKLE

SEQ ID NO: 24 - protein, 893 aa, Homo Sapiens
  MISC_FEATURE (1)..(893): OGTA119 = Swiss Prot Accession No. Q6UKI4, KIAA1228 protein
MTPPSRAEAGVRRSRVPSEGRWRGAEPPGISASTQPASAGRAARHCGAMSGARGEGPEAGAGGAGGRAAPENPGGVLSVELPGLLAQLARSFALLLPVYALGYLGLSFSWVLLALALLAWCRRSRGLKALRLCRALALLEDEERVVRLGVRACDLPAWVHFPDTERAEWLNKTVKHMWPFICQFIEKLFRETIEPAVRGANTHLSTFSFTKVDVGQQPLRINGVKVYTENVDKRQIILDLQISFVGNCEIDLEIKRYFCRAGVKSIQIHGTMRVILEPLIGDMPLVGALSIFFLRKPLLEINWTGLTNLLDVPGLNGLSDTIILDIISNYLVLPNRITVPLVSEVQIAQLRFPVPKGVLRIHFIEAQDLQGKDTYLKGLVKGKSDPYGIIRVGNQIFQSRVIKENLSPKWNEVYEALVYEHPGQELEIELFDEDPDKDDFLGSLMIDLIEVEKERLLDEWFTLDEVPKGKLHLRLEWLTLMPNASNLDKVLTDIKADKDQANDGLSSALLILYLDSARNLPSGKKISSNPNPVVQMSVGHKAQESKIRYKTNEPVWEENFTFFIHNPKRQDLEVEVRDEQHQCSLGNLKVPLSQLLTSEDMTVSQRFQLSNSGPNSTIKMKIALRVLHLEKRERPPDHQHSAQVKRPSVSKEGRKTSIKSHMSGSPGPGGSNTAPSTPVIGGSDKPGMEEKAQPPEAGPQGLHDLGRSSSSLLASPGHISVKEPTPSIASDISLPIATQELRQRLRQLENGTTLGQSPLGQIQLTIRHSSQRNKLIVVVHACRNLIAFSEDGSDPYVRMYLLPDKRRSGRRKTHVSKKTLNPVFDQSFDFSVSLPEVQRRTLDVAVKNSGGFLSKDKGLLGKVLVALASEELAKGWTQWYDLTEDGTRPQAMT

SEQ ID NO: 25 - protein, 1085 aa, Homo Sapiens
  MISC_FEATURE (1)..(1085): OGTA124 = Swiss Prot Accession No. Q9UP95, Solute carrier family 12 member 4
MPHFTVVPVDGPRRGDYDNLEGLSWVDYGERAELDDSDGHGNHRESSPFLSPLEASRGIDYYDRNLALFEEELDIRPKVSSLLGKLVSYTNLTQGAKEHEEAESGEGTRRRAAEAPSMGTLMGVYLPCLQNIFGVILFLRLTWMVGTAGVLQALLIVLICCCCTLLTAISMSAIATNGVVPAGGSYFMISRSLGPEFGGAVGLCFYLGTTFAAAMYILGAIEILLTYIAPPAAIFYPSGAHDTSNATLNNMRVYGTIFLTFMTLVVFVGVKYVNKFASLFLACVIISILSIYAGGIKSIFDPPVFPVCMLGNRTLSRDQFDICAKTAVVDNETVATQLWSFFCHSPNLTTDSCDPYFMLNNVTEIPGIPGAAAGVLQENLWSAYLEKGDIVEKHGLPSADAPSLKESLPLYVVADIATSFTVLVGIFFPSVTGIMAGSNRSGDLRDAQKSIPVGTILAIITTSLVYFSSVVLFGACIEGVVLRDKYGDGVSRNLVVGTLAWPSPWVIVIGSFFSTCGAGLQSLTGAPRLLQAIAKDNIIPFLRVFGHGKVNGEPTWALLLTALIAELGILIASLDMVAPILSMFFLMCYLFVNLACAVQTLLRTPNWRPRFKYYHWALSFLGMSLCLALMFVSSWYYALVAMLIAGMIYKYIEYQGAEKEWGDGIRGLSLSAARYALLRLEEGPPHTKNWRPQLLVLLKLDEDLHVKYPRLLTFASQLKAGKGLTIVGSVIQGSFLESYGEAQAAEQTIKNMMEIEKVKGFCQVVVASKVREGLAHLIQSCGLGGMRHNSVVLGWPYGWRQSEDPRAWKTFIDTVRCTTAAHLALLVPKNIAFYPSNHERYLEGHIDVWWIVHDGGMLMLLPFLLRQHKVWRKCRMRIFTVAQMDDNSIQMKKDLAVFLYHLRLEAEVEVVEMHNSDISAYTYERTLMMEQRSQMLRQMRLTKTEREREAQLVKDRHSALRLESLYSDEEDESAVGADKIQMTWTRDKYMTETWDPSHAPDNFRELVHIKPDQSNVRRMHTAVKLNEVIVTRSHDARLVLLNMPGPPRNSEGDENYMEFLEVLTEGLERVLLVRGGGREVITIYS

SEQ ID NO: 26 - protein, 836 aa, Homo Sapiens [sequence omitted]

SEQ ID NO: 27 - protein, 1038 aa, Homo Sapiens
  MISC_FEATURE (1)..(1038): OGTA156 = Swiss Prot Accession No. P13612, Integrin alpha-4
MFPTESAWLGKRGANPGPEAAVRETVMLLLCLGVPTGRPYNVDTESALLYQGPHNTLFGYSVVLHSHGANRWLLVGAPTANWLANASVINPGAIYRCRIGKNPGQTCEQLQLGSPNGEPCGKTCLEERDNQWLGVTLSRQPGENGSIVTCGHRWKNIFYIKNENKLPTGGCYGVPPDLRTELSKRIAPCYQDYVKKFGENFASCQAGISSFYTKDLIVMGAPGSSYWTGSLFVYNITTNKYKAFLDKQNQVKFGSYLGYSVGAGHFRSQHTTEVVGGAPQHEQIGKAYIFSIDEKELNILHEMKGKKLGSYFGASVCAVDLNADGFSDLLVGAPMQSTIREEGRVFVYINSGSGAVMNAMETNLVGSDKYAARFGESIVNLGDIDNDGFEDVAIGAPQEDDLQGAIYIYNGRADGISSTFSQRIEGLQISKSLSMFGQSISGQIDADNNGYVDVAVGAFRSDSAVLLRTRPVVIVDASLSHPESVNRTKFDCVENGWPSVCIDLTLCFSYKGKEVPGYIVLFYNMSLDVNRKAESPPRFYFSSNGTSDVITGSIQVSSREANCRTHQAFMRKDVRDILTPIQIEAAYHLGPHVISKRSTEEFPPLQPILQQKKEKDIMKKTINFARFCAHENCSADLQVSAKIGFLKPHENKTYLAVGSMKTLMLNVSLFNAGDDAYETTLHVKLPVGLYFIKILELEEKQINCEVTDNSGVVQLDCSIGYIYVDHLSRIDISFLLDVSSLSRAEEDLSITVHATCENEEEMDNLKHSRVTVAIPLKYEVKLTVHGFVNPTSFVYGSNDENEPETCMVEKMNLTFHVINTGNSMAPNVSVEIMVPNSFSPQTDKLFNILDVQTTTGECHFENYQRVCALEQQKSAMQTLKGIVRFLSKTDKRLLYCIKADPHCLNFLCNFGKMESGKEASVHIQLEGRPSILEMDETSALKFEIRATGFPEPNPRVIELNKDENVAHVLLEGLHHQRPKRYFTIVIISSSLLLGLIVLLLISYVMWKAGFFKRQYKSILQEENRRDSWSYINSKSNDD

SEQ ID NO: 28 - protein, 314 aa, Homo Sapiens
  MISC_FEATURE (1)..(314): OGTA159 = Swiss Prot Accession No. Q96HY6, Uncharacterized protein C20orf116
MVAPVWYLVAAALLVGFILFLTRSRGRAASAGQEPLHNEELAGAGRVAQPGPLEPEEPRAGGRPRRRRDLGSRLQAQRRAQRVAWAEADENEEEAVILAQEEEGVEKPAETHLSGKIGAKKLRKLEEKQARKAQREAEEAEREERKRLESQREAEWKKEEERLRLEEEQKEEEERKAREEQAQREHEEYLKLKEAFVVEEEGVGETMTEEQSQSFLTEFINYIKQSKVVLLEDLASQVGLRTQDTINRIQDLLAEGTITGVIDDRGKFIYITPEELAAVANFIRQRGRVSIAELAQASNSLIAWGRESPAQAPA

SEQ ID NO: 29 - protein, 742 aa, Homo Sapiens
  MISC_FEATURE (1)..(742): OGTA168 = Swiss Prot Accession No. Q8WZA4, Collectin placenta 1
MKDDFAEEEEVQSFGYKRFGIQEGTQCTKCKNNWALKFSIILLYILCALLTITVAILGYKVVEKMDNVTGGMETSRQTYDDKLTAVESDLKKLGDQTGKKAISTNSELSTFRSDILDLRQQLREITEKTSKNKDTLEKLQASGDALVDRQSQLKETLENNSFLITTVNKTLQAYNGYVTNLQQDTSVLQGNLQNQMYSHNVVIMNLNNLNLTQVQQRNLITNLQRSVDDTSQAIQRIKNDFQNLQQVFLQAKKDTDWLKEKVQSLQTLAANNSALAKANNDTLEDMNSQLNSFTGQMENITTISQANEQNLKDLQDLHKDAENRTAIKFNQLEERFQLFETDIVNIISNISYTAHHLRTLTSNLNEVRTTCTDTLTKHTDDLTSLNNTLANIRLDSVSLRMQQDLMRSRLDTEVANLSVIMEEMKLVDSKHGQLIKNFTILQGPPGPRGPRGDRGSQGPPGPTGNKGQKGEKGEPGPPGPAGERGPIGPAGPPGERGGKGSKGSQGPKGSRGSPGKPGPQGSSGDPGPPGPPGKEGLPGPQGPPGFQGLQGTVGEPGVPGPRGLPGLPGVPGMPGPKGPPGPPGPSGAVVPLALQNEPTPAPEDNGCPPHWKNFTDKCYYFSVEKEIFEDAKLFCEDKSSHLVFINTREEQQWIKKQMVGRESHWIGLTDSERENEWKWLDGTSPDYKNWKAGQPDNWGHGHGPGEDCAGLIYAGQWNDFQCEDVNNFICEKDRETVLSSAL

SEQ ID NO: 30 - protein, 1163 aa, Homo Sapiens
  MISC_FEATURE (1)..(1163): OGTA169 = Swiss Prot Accession No. P20702, Integrin alpha-X
MTRTRAALLLFTALATSLGFNLDTEELTAFRVDSAGFGDSVVQYANSWVVVGAPQKITAANQTGGLYQCGYSTGACEPIGLQVPPEAVNMSLGLSLASTTSPSQLLACGPTVHHECGRNMYLTGLCFLLGPTQLTQRLPVSRQECPRQEQDIVFLIDGSGSISSRNFATMMNFVRAVISQFQRPSTQFSLMQFSNKFQTHFTFEEFRRTSNPLSLLASVHQLQGFTYTATAIQNVVHRLFHASYGARRDATKILIVITDGKKEGDSLDYKDVIPMADAAGIIRYAIGVGLAFQNRNSWKELNDIASKPSQEHIFKVEDFDALKDIQNQLKEKIFAIEGTETTSSSSFELEMAQEGFSAVFTPDGPVLGAVGSFTWSGGAFLYPPNMSPTFINMSQENVDMRDSYLGYSTELALWKGVQSLVLGAPRYQHTGKAVIFTQVSRQWRMKAEVTGTQIGSYFGASLCSVDVDTDGSTDLVLIGAPHYYEQTRGGQVSVCPLPRGWRRWWCDAVLYGEQGHPWGRFGAALTVLGDVNGDKLTDVVIGAPGEEENRGAVYLFHGVLGPSISPSHSQRIAGSQLSSRLQYFGQALSGGQDLTQDGLVDLAVGARGQVLLLRTRPVLWVGVSMQFIPAEIPRSAFECREQVVSEQTLVQSNICLYIDKRSKNLLGSRDLQSSVTLDLALDPGRLSPRATFQETKNRSLSRVRVLGLKAHCENFNLLLPSCVEDSVTPITLRLNFTLVGKPLLAFRNLRPMLAADAQRYFTASLPFEKNCGADHICQDNLGISFSFPGLKSLLVGSNLELNAEVMVWNDGEDSYGTTITFSHPAGLSYRYVAEGQKQGQLRSLHLTCDSAPVGSQGTWSTSCRINHLIFRGGAQITFLATFDVSPKAVLGDRLLLTANVSSENNTPRTSKTTFQLELPVKYAVYTVVSSHEQFTKYLNFSESEEKESHVAMHRYQVNNLGQRDLPVSINFWVPVELNQEAVWMDVEVSHPQNPSLRCSSEKIAPPASDFLAHIQKNPVLDCSIAGCLRFRCDVPSFSVQEELDFTLKGNLSFGWVRQILQKKVSVVSVAEITFDTSVYSQLPGQEAFMRAQTTTVLEKYKVHNPTPLIVGSSIGGLLLLALITAVLYKVGFFKRQYKEMMEEANGQIAPENGTQTPSPPSEK

SEQ ID NO: 31 - protein, 495 aa, Homo Sapiens
  MISC_FEATURE (1)..(495): OGTA174 = Swiss Prot Accession No. P06127, T-cell surface glycoprotein CD5
MPMGSLQPLATLYLLGMLVASCLGRLSWYDPDFQARLTRSNSKCQGQLEVYLKDGWHMVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPAGLAAGTVASIILALVLLVVLLVVCGPLAYKKLVKKFRQKKQRQWIGPTGMNQNMSFHRNHTATVRSHAENPTASHVDNEYSQPPRNSRLSAYPALEGVLHRSSMQPDNSSDSDYDLHGAQRL

SEQ ID NO: 32 - protein, 359 aa, Homo Sapiens
  MISC_FEATURE (1)..(359): OGTA176 = Swiss Prot Accession No. P21854, B-cell differentiation antigen CD72 homolog
MAEAITYADLRFVKAPLKKSISSRLGQDPGADDDGEITYENVQVPAVLGVPSSLASSVLGDKAAVKSEQPTASWRAVTSPAVGRILPCRTTCLRYLLLGLLLTCLLLGVTAICLGVRYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 33 - protein, 510 aa, Homo Sapiens
  MISC_FEATURE (1)..(510): OGTA177 = Swiss Prot Accession No. P49961, Ectonucleoside triphosphate diphosphohydrolase 1
MEDTKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 34 - protein, 976 aa, Homo Sapiens
  MISC_FEATURE (1)..(976): OGTA197 = Swiss Prot Accession No. P29317, Ephrin type-A receptor 2
MELQAARACFALLWGCALAAAAAAQGKEVVLLDFAAAGGELGWLTHPYGKGWDLMQNIMNDMPIYMYSVCNVMSGDQDNWLRTNWVYRGEAERNNFELNFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 35 - protein, 829 aa, Homo Sapiens [sequence omitted]

SEQ ID NO: 36 - protein, 790 aa, Homo Sapiens
  MISC_FEATURE (1)..(790): OGTA203 = Swiss Prot Accession No. P55285, Cadherin-6
MRTYRYFLLLFWVGQPYPTLSTPLSKRTSGFPAKKRALELSGNSKNELNRSKRSWMWNQFFLLEEYTGSDYQYVGKLHSDQDRGDGSLKYILSGDGAGDLFIINENTGDIQATKRLDREEKPVYILRAQAINRRTGRPVEPESEFIIKIHDINDNEPIFTKEVYTATVPEMSDVGTFVVQVTATDADDPTYGNSAKVVYSILQGQPYFSVESETGIIKTALLNMDRENREQYQVVIQAKDMGGQMGGLSGTTTVNITLTDVNDNPPRFPQSTYQFKTPESSPPGTPIGRIKASDADVGENAEIEYSITDGEGLDMFDVITDQETQEGIITVKKLLDFEKKKVYTLKVEASNPYVEPRFLYLGPFKDSATVRIVVEDVDEPPVFSKLAYILQIREDAQINTTIGSVTAQDPDAARNPVKYSVDRHTDMDRIFNIDSGNGSIFTSKLLDRETLLWHNITVIATEINNPKQSSRVPLYIKVLDVNDNAPEFAEFYETFVCEKAKADQLIQTLHAVDKDDPYSGHQFSFSLAPEAASGSNFTIQDNKDNTAGILTRKNGYNRHEMSTYLLPVVISDNDYPVQSSTGTVTVRVCACDHHGNMQSCHAEALIHPTGLSTGALVAILLCIVILLVTVVLFAALRRQRKKEPLIISKEDIRDNIVSYNDEGGGEEDTQAFDIGTLRNPEAIEDNKLRRDIVPEALFLPRRTPTARDNTDVRDFINQRLKENDTDPTAPPYDSLATYAYEGTGSVADSLSSLESVTTDADQDYDYLSDWGPRFKKLADMYGGVDSDKDS

SEQ ID NO: 37 - protein, 209 aa, Homo Sapiens
  MISC_FEATURE (1)..(209): OGTA206 = Swiss Prot Accession No. O14493, Claudin-4
MASMGLQVMGIALAVLGWLAVMLCCALPMWRVTAFIGSNIVTSQTIWEGLWMNCVVQSTGQMQCKVYDSLLALPQDLQAARALVIISIIVAALGVLLSVVGGKCTNCLEDESAKAKTMIVAGVVFLLAGLMVIVPVSWTAHNIIQDFYNPLVASGQKREMGASLYVGWAASGLLLLGGGLLCCNCPPRTDKPYSAKYSAARSAAASNYV

SEQ ID NO: 38 - protein, 1821 aa, Homo Sapiens
  MISC_FEATURE (1)..(1821): OGTA213 = Swiss Prot Accession No. Q14767, Latent-transforming growth factor beta-binding protein 2
MRPRTKARSPGRALRNPWRGFLPLTLALFVGAGHAQRDPVGRYEPAGGDANRLRRPGGSYPAAAAAKVYSLFREQDAPVAGLQPVERAQPGWGSPRRPTEAEARRPSRAQQSRRVQPPAQTRRSTPLGQQQPAPRTRAAPALPRLGTPQRSGAAPPTPPRGRLTGRNVCGGQCCPGWTTANSTNHCIKPVCEPPCQNRGSCSRPQLCVCRSGFRGARCEEVIPDEEFDPQNSRLAPRRWAERSPNLRRSSAAGEGTLARAQPPAPQSPPAPQSPPAGTLSGLSQTHPSQQHVGLSRTVRLHPTATASSQLSSNALPPGPGLEQRDGTQQAVPLEHPSSPWGLNLTEKIKKIKIVFTPTICKQTCARGHCANSCERGDTTTLYSQGGHGHDPKSGFRIYFCQIPCLNGGRCIGRDECWCPANSTGKFCHLPIPQPDREPPGRGSRPRALLEAPLKQSTFTLPLSNQLASVNPSLVKVHIHHPPEASVQIHQVAQVRGGVEEALVENSVETRPPPWLPASPGHSLWDSNNIPARSGEPPRPLPPAAPRPRGLLGRCYLNTVNGQCANPLLELTTQEDCCGSVGAFWGVTLCAPCPPRPASPVIENGQLECPQGYKRLNLTHCQDINECLTLGLCKDAECVNTRGSYLCTCRPGLMLDPSRSRCVSDKAISMLQGLCYRSLGPGTCTLPLAQRITKQICCCSRVGKAWGSECEKCPLPGTEAFREICPAGHGYTYASSDIRLSMRKAEEEELARPPREQGQRSSGALPGPAERQPLRVVTDTWLEAGTIPDKGDSQAGQVTTSVTHAPAWVTGNATTPPMPEQGIAEIQEEQVTPSTDVLVTLSTPGIDRCAAGATNVCGPGTCVNLPDGYRCVCSPGYQLHPSQAYCTDDNECLRDPCKGKGRCINRVGSYSCFCYPGYTLATSGATQECQDINECEQPGVCSGGQCTNTEGSYHCECDQGYIMVRKGHCQDINECRHPGTCPDGRCVNSPGSYTCLACEEGYRGQSGSCVDVNECLTPGVCAHGKCTNLEGSFRCSCEQGYEVTSDEKGCQDVDECASRASCPTGLCLNTEGSFACSACENGYWVNEDGTACEDLDECAFPGVCPSGVCTNTAGSFSCKDCDGGYRPSPLGDSCEDVDECEDPQSSCLGGECKNTVGSYQCLCPQGFQLANGTVCEDVNECMGEEHCAPHGECLNSHGSFFCLCAPGFVSAEGGTSCQDVDECATTDPCVGGHCVNTEGSFNCLCETGFQPSPESGECVDIDECEDYGDPVCGTWKCENSPGSYRCVLGCQPGFHMAPNGDCIDIDECANDTMCGSHGFCDNTDGSFRCLCDQGFEISPSGWDCVDVNECELMLAVCGAALCENVEGSFLCLCASDLEEYDAQEGHCRPRGAGGQSMSEAPTGDHAPAPTRMDCYSGQKGHAPCSSVLGRNTTQAECCCTQGATWGDACDLCPSEDSAEFSEICPSGKGYIPVEGAWTFGQTMYTDADECVIFGPGLCPNGRCLNTVPGYVCLCNPGFHYDASHKKCEDHDECQDLACENGECVNTEGSFHCFCSPPLTLDLSQQRCMNSTSSTEDLPDHDIHMDICWKKVTNDVCSEPLRGHRTTYTECCCQDGKAWSQQCALCPPRSSEVYAQLCNVARIEAEREAGVHFRPGYEYGPGPDDLHYSIYGPDGAPFYNYLGPEDTVPEPAFPNTAGHSADRTPILESPLQPSELQPHYVASHPEPPAGFEGLQAEECGILNGCENGRCVRVREGYTCDCFEGFQLDAAHMACVDVNECDDLNGPAVLCVHGYCENTEGSYRCHCSPGYVAEAGPPHCTAKE

SEQ ID NO: 39 - protein, 512 aa, Homo Sapiens
  MISC_FEATURE (1)..(512): OGTA214 = Swiss Prot Accession No. Q9H3R2, Mucin-13
MKAIIHLTLLALLSVNTATNQGNSADAVTTTETATSGPTVAAADTTETNFPETASTTANTPSFPTATSPAPPIISTHSSSTIPTPAPPIISTHSSSTIPIPTAADSESTTNVNSLATSDIITASSPNDGLITMVPSETQSNNEMSPTTEDNQSSGPPTGTALLETSTLNSTGPSNPCQDDPCADNSLCVKLHNTSFCLCLEGYYYNSSTCKKGKVFPGKISVTVSETFDPEEKHSMAYQDLHSEITSLFKDVFGTSVYGQTVILTVSTSLSPRSEMRADDKFVNVTIVTILAETTSDNEKTVTEKINKAIRSSSSNFLNYDLTLRCDYYGCNQTADDCLNGLACDCKSDLQRPNPQSPFCVASSLKCPDACNAQHKQCLIKKSGGAPECACVPGYQEDANGNCQKCAFGYSGLDCKDKFQLILTIVGTIAGIVILSMIIALIVTARSNNKTKHIEEENLIDEDFQNLKLRSTGFTNLGAEGSVFPKVRITASRDSQMQNPYSRHSSMPRPDY

SEQ ID NO: 40 - protein, 1212 aa, Homo Sapiens
  MISC_FEATURE (1)..(1212): OGTA216 = Swiss Prot Accession No. P55011, Solute carrier family 12 member 2
MEPRPTAPSSGAPGLAGVGETPSAAALAAARVELPGTAVPSVPEDAAPASRDGGGVRDEGPAAAGDGLGRPLGPTPSQSRFQVDLVSENAGRAAAAAAAAAAAAAAAGAGAGAKQTPADGEASGESEPAKGSEEAKGRFRVNFVDPAASSSAEDSLSDAAGVGVDGPNVSFQNGGDTVLSEGSSLHSGGGGGSGHHQHYYYDTHTNTYYLRTFGHNTMDAVPRIDHYRHTAAQLGEKLLRPSLAELHDELEKEPFEDGFANGEESTPTRDAVVTYTAESKGVVKFGWIKGVLVRCMLNIWGVMLFIRLSWIVGQAGIGLSVLVIMMATVVTTITGLSTSAIATNGFVRGGGAYYLISRSLGPEFGGAIGLIFAFANAVAVAMYVVGFAETVVELLKEHSILMIDEINDIRIIGAITVVILLGISVAGMEWEAKAQIVLLVILLLAIGDFVIGTFIPLESKKPKGFFGYKSEIFNENFGPDFREEETFFSVFAIFFPAATGILAGANISGDLADPQSAIPKGTLLAILITTLVYVGIAVSVGSCVVRDATGNVNDTIVTELTNCTSAACKLNFDFSSCESSPCSYGLMNNFQVMSMVSGFTPLISAGIFSATLSSALASLVSAPKIFQALCKDNIYPAFQMFAKGYGKNNEPLRGYILTFLIALGFILIAELNVIAPIISNFFLASYALINFSVFHASLAKSPGWRPAFKYYNMWISLLGAILCCIVMFVINWWAALLTYVIVLGLYIYVTYKKPDVNWGSSTQALTYLNALQHSIRLSGVEDHVKNFRPQCLVMTGAPNSRPALLHLVHDFTKNVGLMICGHVHMGPRRQAMKEMSIDQAKYQRWLIKNKMKAFYAPVHADDLREGAQYLMQAAGLGRMKPNTLVLGFKKDWLQADMRDVDMYINLFHDAFDIQYGVVVIRLKEGLDISHLQGQEELLSSQEKSPGTKDVVVSVEYSKKSDLDTSKPLSEKPITHKVEEEDGKTATQPLLKKESKGPIVPLNVADQKLLEASTQFQKKQGKNTIDVWWLFDDGGLTLLIPYLLTTKKKWKDCKIRVFIGGKINRIDHDRRAMATLLSKFRIDFSDIMVLGDINTKPKKENIIAFEEIIEPYRLHEDDKEQDIADKMKEDEPWRITDNELELYKTKTYRQIRLNELLKEHSSTANIIVMSLPVARKGAVSSALYMAWLEALSKDLPPILLVRGNHQSVLTFYS

SEQ ID NO: 41 - protein, 411 aa, Homo Sapiens
  MISC_FEATURE (1)..(411): OGTA222 = Swiss Prot Accession No. P16444, Dipeptidase 1
MWSGWWLWPLVAVCTADFFRDEAERIMRDSPVIDGHNDLPWQLLDMFNNRLQDERANLTTLAGTHTNIPKLRAGFVGGQFWSVYTPCDTQNKDAVRRTLEQMDVVHRMCRMYPETFLYVTSSAGIRQAFREGKVASLIGVEGGHSIDSSLGVLRALYQLGMRYLTLTHSCNTPWADNWLVDTGDSEPQSQGLSPFGQRVVKELNRLGVLIDLAHVSVATMKATLQLSRAPVIFSHSSAYSVCASRRNVPDDVLRLVKQTDSLVMVNFYNNYISCTNKANLSQVADHLDHIKEVAGARAVGFGGDFDGVPRVPEGLEDVSKYPDLIAELLRRNWTEAEVKGALADNLLRVFEAVEQASNLTQAPEEEPIPLDQLGGSCRTHYGYSSGASSLHRHWGLLLASLAPLVLCLSLL

SEQ ID NO: 42 - protein, 739 aa, Homo Sapiens
  MISC_FEATURE (1)..(739): OGTA236 = Swiss Prot Accession No. P50443, Sulfate transporter
MSSESKEQHNVSPRDSAEGNDSYPSGIHLELQRESSTDFKQFETNDQCRPYHRILIERQEKSDTNFKEFVIKKLQKNCQCSPAKAKNMILGFLPVLQWLPKYDLKKNILGDVMSGLIVGILLVPQSIAYSLLAGQEPVYGLYTSFFASIIYFLLGTSRHISVGIFGVLCLMIGETVDRELQKAGYDNAHSAPSLGMVSNGSTLLNHTSDRICDKSCYAIMVGSTVTFIAGVYQVAMGFFQVGFVSVYLSDALLSGFVTGASFTILTSQAKYLLGLNLPRTNGVGSLITTWIHVFRNIHKTNLCDLITSLLCLLVLLPTKELNEHFKSKLKAPIPIELVVVVAATLASHFGKLHENYNSSIAGHIPTGFMPPKVPEWNLIPSVAVDAIAISIIGFAITVSLSEMFAKKHGYTVKANQEMYAIGFCNIIPSFFHCFTTSAALAKTLVKESTGCHTQLSGVVTALVLLLVLLVIAPLFYSLQKSVLGVITIVNLRGALRKFRDLPKMWSISRMDTVIWFVTMLSSALLSTEIGLLVGVCFSIFCVILRTQKPKSSLLGLVEESEVFESVSAYKNLQTKPGIKIFRFVAPLYYINKECFKSALYKQTVNPILIKVAWKKAAKRKIKEKVVTLGGIQDEMSVQLSHDPLELHTIVIDCSAIQFLDTAGIHTLKEVRRDYEAIGIQVLLAQCNPTVRDSLTNGEYCKKEEENLLFYSVYEAMAFAEVSKNQKGVCVPNGLSLSSD

SEQ ID NO: 43 - protein, 802 aa, Homo Sapiens
  MISC_FEATURE (1)..(802): OGTA237 = Swiss Prot Accession No. P22455, Fibroblast growth factor receptor 4
MRLLLALLGVLLSVPGPPVLSLEASEEVELEPCLAPSLEQQEQELTVALGQPVRLCCGRAERGGHWYKEGSRLAPAGRVRGWRGRLEIASFLPEDAGRYLCLARGSMIVLQNLTLITGDSLTSSNDDEDPKSHRDPSNRHSYPQQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVLPEEDPTWTAAAPEARYTDIILYASGSLALAVLLLLAGLYRGQALHGRHPRPPATVQKLSRFPLARQFSLESGSSGKSSSSLVRGVRLSSSGPALLAGLVSLDLPLDPLWEFPRDRLVLGKPLGEGCFGQVVRAEAFGMDPARPDQASTVAVKMLKDNASDKDLADLVSEMEVMKLIGRHKNIINLLGVCTQEGPLYVIVECAAKGNLREFLRARRPPGPDLSPDGPRSSEGPLSFPVLVSCAYQVARGMQYLESRKCIHRDLAARNVLVTEDNVMKIADFGLARGVHHIDYYKKTSNGRLPVKWMAPEALFDRVYTHQSDVWSFGILLWEIFTLGGSPYPGIPVEELFSLLREGHRMDRPPHCPPELYGLMRECWHAAPSQRPTFKQLVEALDKVLLAVSEEYLDLRLTFGPYSPSGGDASSTCSSSDSVFSHDPLPLGSSSFPFGSGVQT

SEQ ID NO: 44 - protein, 1257 aa, Homo Sapiens
  MISC_FEATURE (1)..(1257): OGTA247 = Swiss Prot Accession No. P32004, Neural cell adhesion molecule L1
MVVALRYVWPLLLCSPCLLIQIPEEYEGHHVMEPPVITEQSPRRLVVFPTDDISLKCEASGKPEVQFRWTRDGVHFKPKEELGVTVYQSPHSGSFTITGNNSNFAQRFQGIYRCFASNKLGTAMSHEIRLMAEGAPKWPKETVKPVEVEEGESVVLPCNPPPSAEPLRIYWMNSKILHIKQDERVTMGQNGNLYFANVLTSDNHSDYICHAHFPGTRTIIQKEPIDLRVKATNSMIDRKPRLLFPTNSSSHLVALQGQPLVLECIAEGFPTPTIKWLRPSGPMPADRVTYQNHNKTLQLLKVGEEDDGEYRCLAENSLGSARHAYYVTVEAAPYWLHKPQSHLYGPGETARLDCQVQGRPQPEVTWRINGIPVEELAKDQKYRIQRGALILSNVQPSDTMVTQCEARNRHGLLLANAYIYVVQLPAKILTADNQTYMAVQGSTAYLLCKAFGAPVPSVQWLDEDGTTVLQDERFFPYANGTLGIRDLQANDTGRYFCLAANDQNNVTIMANLKVKDATQITQGPRSTIEKKGSRVTFTCQASFDPSLQPSITWRGDGRDLQELGDSDKYFIEDGRLVIHSLDYSDQGNYSCVASTELDVVESRAQLLVVGSPGPVPRLVLSDLHLLTQSQVRVSWSPAEDHNAPIEKYDIEFEDKEMAPEKWYSLGKVPGNQTSTTLKLSPYVHYTFRVTAINKYGPGEPSPVSETVVTPEAAPEKNPVDVKGEGNETTNMVITWKPLRWMDWNAPQVQYRVQWRPQGTRGPWQEQIVSDPFLVVSNTSTFVPYEIKVQAVNSQGKGPEPQVTIGYSGEDYPQAIPELEGIEILNSSAVLVKWRPVDLAQVKGHLRGYNVTYWREGSQRKHSKRHIHKDHVVVPANTTSVILSGLRPYSSYHLEVQAFNGRGSGPASEFTFSTPEGVPGHPEALHLECQSNTSLLLRWQPPLSHNGVLTGYVLSYHPLDEGGKGQLSFNLRDPELRTHNLTDLSPHLRYRFQLQATTKEGPGEAIVREGGTMALSGISDFGNISATAGENYSVVSWVPKEGQCNFRFHILFKALGEEKGGASLSPQYVSYNQSSYTQWDLQPDTDYEIHLFKERMFRHQMAVKTNGTGRVRLPPAGFATEGWFIGFVSAIILLLLVLLILCFIKRSKGGKYSVKDKEDTQVDSEARPMKDETFGEYRSLESDNEEKAFGSSQPSLNGDIKPLGSDDSLADYGGSVDVQFNEDGSFIGQYSGKKEKEAAGGNDSSGATSPINPAVALE

SEQ ID NO: 45 - protein, 864 aa, Homo Sapiens [sequence omitted]

SEQ ID NO: 46 - protein, 176 aa, Homo Sapiens
  MISC_FEATURE (1)..(176): OGTA249 = Swiss Prot Accession No. Q969L2, Protein MAL2
MSAGGASVPPPPNPAVSFPPPRVTLPAGPDILRTYSGAFVCLEILFGGLVWILVASSNVPLPLLQGWVMFVSVTAFFFSLLFLGMFLSGMVAQIDANWNFLDFAYHFTVFVFYFGAFLLEAAATSLHDLHCNTTITGQPLLSDNQYNINVAASIFAFMTTACYGCSLGLALRRWRP

SEQ ID NO: 47 - protein, 725 aa, Homo Sapiens
  MISC_FEATURE (1)..(725): OGTA257 = Swiss Prot Accession No. Q9H1D0, Transient receptor potential cation channel subfamily V member 6
MGLSLPKEKGLILCLWSKFCRWFQRRESWAQSRDEQNLLQQKRIWESPLLLAAKDNDVQALNKLLKYEDCKVHQRGAMGETALHIAALYDNLEAAMVLMEAAPELVFEPMTSELYEGQTALHIAVVNQNMNLVRALLARRASVSARATGTAFRRSPCNLIYFGEHPLSFAACVNSEEIVRLLIEHGADIRAQDSLGNTVLHILILQPNKTFACQMYNLLLSYDRHGDHLQPLDLVPNHQGLTPFKLAGVEGNTVMFQHLMQKRKHTQWTYGPLTSTLYDLTEIDSSGDEQSLLELIITTKKREARQILDQTPVKELVSLKWKRYGRPYFCMLGAIYLLYIICFTMCCIYRPLKPRTNNRTSPRDNTLLQQKLLQEAYMTPKDDIRLVGELVTVIGAIIILLVEVPDIFRMGVTRFFGQTILGGPFHVLIITYAFMVLVTMVMRLISASGEVVPMSFALVLGWCNVMYFARGFQMLGPFTIMIQKMIFGDLMRFCWLMAVVILGFASAFYIIFQTEDPEELGHFYDYPMALFSTFELFLTIIDGPANYNVDLPFMYSITYAAFAIIATLLMLNLLIAMMGDTHWRVAHERDELWRAQIVATTVMLERKLPRCLWPRSGICGREYGLGDRWFLRVEDRQDLNRQRIQRYAQAFHTRGSEDLDKDSVEKLELGCPFSPHLSLPMPSVSRSTSRSSANWERLRQGTLRRDLRGIINRGLEDGESWEYQI

SEQ ID NO: 48 - protein, 1898 aa, Homo Sapiens
  MISC_FEATURE (1)..(1898): OGTA271 = Swiss Prot Accession No. Q5T021, Protein tyrosine phosphatase, receptor type, F
MAPEPAPGRTMVPLVPALVMLGLVAGAHGDSKPVFIKVPEDQTGLSGGVASFVCQATGEPKPRITWMKKGKKVSSQRFEVIEFDDGAGSVLRIQPLRVQRDEAIYECTATNSLGEINTSAKLSVLEEEQLPPGFPSIDMGPQLKVVEKARTATMLCAAGGNPDPEISWFKDFLPVDPATSNGRIKQLRSGALQIESSEESDQGKYECVATNSAGTRYSAPANLYVRVRRVAPRFSIPPSSQEVMPGGSVNLTCVAVGAPMPYVKWMMGAEELTKEDEMPVGRNVLELSNVVRSANYTCVAISSLGMIEATAQVTVKALPKPPIDLVVTETTATSVTLTWDSGNSEPVTYYGIQYRAAGTEGPFQEVDGVATTRYSIGGLSPFSEYAFRVLAVNSIGRGPPSEAVRARTGEQAPSSPPRRVQARMLSASTMLVQWEPPEEPNGLVRGYRVYYTPDSRRPPNAWHKHNTDAGLLTTVGSLLPGITYSLRVLAFTAVGDGPPSPTIQVKTQQGVPAQPADFQAEVESDTRIQLSWLLPPQERIIMYELVYWAAEDEDQQHKVTFDPTSSYTLEDLKPDTLYRFQLAARSDMGVGVFTPTIEARTAQSTPSAPPQKVMCVSMGSTTVRVSWVPPPADSRNGVITQYSVAYEAVDGEDRGRHVVDGISREHSSWDLVGLEKWTEYRVWVRAHTDVGPGPESSPVLVRTDEDVPSGPPRKVEVEPLNSTAVHVYWKLPVPSKQHGQIRGYQVTYVRLENGEPRGLPIIQDVMLAEAQETTISGLTPETTYSVTVAAYTTKGDGARSKPKIVTTTGAVPGRPTMMISTTAMNTALLQWHPPKELPGELLGYRLQYCRADEARPNTIDFGKDDQHFTVTGLHKGTTYIFRLAAKNRAGLGEEFEKEIRTPEDLPSGFPQNLHVTGLTTSTTELAWDPPVLAERNGRIISYTVVFRDINSQQELQNITTDTRFTLTGLKPDTTYDIKVRAWTSKGSGPLSPSIQSRTMPVEQVFAKNFRVAAAMKTSVLLSWEVPDSYKSAVPFKILYNGQSVEVDGHSMRKLIADLQPNTEYSFVLMNRGSSAGGLQHLVSIRTAPDLLPHKPLPASAYIEDGRFDLSMPHVQDPSLVRWFYIVVVPIDRVGGSMLTPRWSTPEELELDELLEAIEQGGEEQRRRRRQAERLKPYVAAQLDVLPETFTLGDKKNYRGFYNRPLSPDLSYQCFVLASLKEPMDQKRYASSPYSDEIVVQVTPAQQQEEPEMLWVTGPVLAVILIILIVIAILLFKRKRTHSPSSKDEQSIGLKDSLLAHSSDPVEMRRLNYQTPGMRDHPPIPITDLADNIERLKANDGLKFSQEYESIDPGQQFTWENSNLEVNKPKNRYANVIAYDHSRVILTSIDGVPGSDYINANYIDGYRKQNAYIATQGPLPETMGDFWRMVWEQRTATVVMMTRLEEKSRVKCDQYWPARGTETCGLIQVTLLDTVELATYTVRTFALHKSGSSEKRELRQFQFMAWPDHGVPEYPTPILAFLRRVKACNPLDAGPMVVHCSAGVGRTGCFIVIDAMLERMKHEKTVDIYGHVTCMRSQRNYMVQTEDQYVFIHEALLEAATCGHTEVPARNLYAHIQKLGQVPPGESVTAMELEFKLLASSKAHTSRFISANLPCNKFKNRLVNIMPYELTRVCLQPIRGVEGSDYINASFLDGYRQQKAYIATQGPLAESTEDFWRMLWEHNSTIIVMLTKLREMGREKCHQYWPAERSARYQYFVVDPMAEYNMPQYILREFKVTDARDGQSRTIRQFQFTDWPEQGVPKTGEGFIDFIGQVHKTKEQFGQDGPITVHCSAGVGRTGVFITLSIVLERMRYEGVVDMFQTVKTLRTQRPAMVQTEDQYQLCYRAALEYLGSFDHYAT

SEQ ID NO: 49 - protein, 22 aa, Homo Sapiens
AAAAAAAAAAAAAAAGAGAGAK

SEQ ID NO: 50 - protein, 18 aa, Homo Sapiens
AAGTEGPFQEVDGVATTR

SEQ ID NO: 51 - protein, 14 aa, Homo Sapiens
AALEYLGSFDHYAT

SEQ ID NO: 52 - protein, 15 aa, Homo Sapiens
AANDPFTIVHGNTGK

SEQ ID NO: 53 - protein, 19 aa, Homo Sapiens
AASAGQEPLHNEELAGAGR

SEQ ID NO: 54 - protein, 8 aa, Homo Sapiens
AATYHVDR

SEQ ID NO: 55 - protein, 20 aa, Homo Sapiens
ACFALLWGCALAAAAAAQGK

SEQ ID NO: 56 - protein, 20 aa, Homo Sapiens
ACNPLDAGPMVVHCSAGVGR

SEQ ID NO: 57 - protein, 8 aa, Homo Sapiens
ADAAPDEK

SEQ ID NO: 58 - protein, 13 aa, Homo Sapiens
ADEARPNTIDFGK

SEQ ID NO: 59 - protein, 7 aa, Homo Sapiens
ADFLIFR

SEQ ID NO: 60 - protein, 15 aa, Homo Sapiens
ADFVLAANSYDLAIK

SEQ ID NO: 61 - protein, 11 aa, Homo Sapiens
ADGISSTFSQR

SEQ ID NO: 62 - protein, 13 aa, Homo Sapiens
AELDDSDGHGNHR

SEQ ID NO: 63 - protein, 17 aa, Homo Sapiens
AEPESETSILLSWTPPR

SEQ ID NO: 64 - protein, 6 aa, Homo Sapiens
AEWLNK

SEQ ID NO: 65 - protein, 24 aa, Homo Sapiens
AFGAPVPSVQWLDEDGTTVLQDER

SEQ ID NO: 66 - protein, 9 aa, Homo Sapiens
AFMIIQEQR

SEQ ID NO: 67 - protein, 21 aa, Homo Sapiens
AFSSDLISIHSLADVEVVVTK

SEQ ID NO: 68 - protein, 10 aa, Homo Sapiens
AFVNCDENSR

SEQ ID NO: 69 - protein, 12 aa, Homo Sapiens
AFYAPVHADDLR

SEQ ID NO: 70 - protein, 15 aa, Homo Sapiens
AGEITSDGLSFLFLK

SEQ ID NO: 71 - protein, 9 aa, Homo Sapiens
AGLGEEFEK

SEQ ID NO: 72 - protein, 17 aa, Homo Sapiens
AHTDVGPGPESSPVLVR

SEQ ID NO: 73 - protein, 7 aa, Homo Sapiens
AINDGFR

SEQ ID NO: 74 - protein, 12 aa, Homo Sapiens
AISTNSELSTFR

SEQ ID NO: 75 - protein, 10 aa, Homo Sapiens
ALALLEDEER

SEQ ID NO: 76 - protein, 14 aa, Homo Sapiens
ALGRPGPPFNITPR

SEQ ID NO: 77 - protein, 8 aa, Homo Sapiens
ALYQLGMR

SEQ ID NO: 78 - protein, 8 aa, Homo Sapiens
AMATLLSK

SEQ ID NO: 79 - protein, 26 aa, Homo Sapiens
ANLTNFPENGTFVVNIAQLSQDDSGR

SEQ ID NO: 80 - protein, 28 aa, Homo Sapiens
APQDPASMPCTRPPSAPHYLTAVGMGAK

SEQ ID NO: 81 - protein, 8 aa, Homo Sapiens
AQDPGEPR

SEQ ID NO: 82 - protein, 14 aa, Homo Sapiens
AQLLVVGSPGPVPR

SEQ ID NO: 83 - protein, 16 aa, Homo Sapiens
AQPPEAGPQGLHDLGR

SEQ ID NO: 84 - protein, 13 aa, Homo Sapiens
AQQEFATGVMSNK

SEQ ID NO: 85 - protein, 6 aa, Homo Sapiens
AQYEGR

SEQ ID NO: 86 - protein, 16 aa, Homo Sapiens
ASVDSGSSEEQGGSSR

SEQ ID NO: 87 - protein, 15 aa, Homo Sapiens
ASVSFLNFNLSNCER

SEQ ID NO: 88 - protein, 10 aa, Homo Sapiens
ATDFDVLSYK

SEQ ID NO: 89 - protein, 11 aa, Homo Sapiens
ATDFDVLSYKK

SEQ ID NO: 90 - protein, 10 aa, Homo Sapiens
ATGFPEPNPR

SEQ ID NO: 91 - protein, 7 aa, Homo Sapiens
ATLQLSR

SEQ ID NO: 92 - protein, 8 aa, Homo Sapiens
ATNSMIDR

SEQ ID NO: 93 - protein, 18 aa, Homo Sapiens
ATQFTGATGAIMTTETTK

SEQ ID NO: 94 - protein, 17 aa, Homo Sapiens
AVDEQGWEYSITIPPER

SEQ ID NO: 95 - protein, 13 aa, Homo Sapiens
AVGFGGDFDGVPR

SEQ ID NO: 96 - protein, 9 aa, Homo Sapiens
AVIFTQVSR

SEQ ID NO: 97 - protein, 21 aa, Homo Sapiens
AVISQFQRPSTQFSLMQFSNK

SEQ ID NO: 98 - protein, 18 aa, Homo Sapiens
AYIATQGPLAESTEDFWR

SEQ ID NO: 99 - protein, 9 aa, Homo Sapiens
AYIFSIDEK

SEQ ID NO: 100 - protein, 17 aa, Homo Sapiens
CDVPSFSVQEELDFTLK

SEQ ID NO: 101 - protein, 12 aa, Homo Sapiens
CEASGKPEVQFR

SEQ ID NO: 102 - protein, 11 aa, Homo Sapiens
CEHHSLYGAAR

SEQ ID NO: 103 - protein, 25 aa, Homo Sapiens
CHIQASGRPQCSCMPGWTGEQCQLR

SEQ ID NO: 104 - protein, 7 aa, Homo Sapiens
CIIWNTR

SEQ ID NO: 105 - protein, 11 aa, Homo Sapiens
CLAENSLGSAR

SEQ ID NO: 106 - protein, 13 aa, Homo Sapiens
CMLNIWGVMLFIR

SEQ ID NO: 107 - protein, 10 aa, Homo Sapiens
CQGQLEVYLK

SEQ ID NO: 108 - protein, 13 aa, Homo Sapiens
CSMLIASNETWKK

SEQ ID NO: 109 - protein, 10 aa, Homo Sapiens
DATQITQGPR

SEQ ID NO: 110 - protein, 11 aa, Homo Sapiens
DAVVTYTAESK

SEQ ID NO: 111 - protein, 12 aa, Homo Sapiens
DDQHFTVTGLHK

SEQ ID NO: 112 - protein, 21 aa, Homo Sapiens
DEAIYECTATNSLGEINTSAK

SEQ ID NO: 113 - protein, 14 aa, Homo Sapiens
DEEEEPPSMTQLLR

SEQ ID NO: 114 - protein, 23 aa, Homo Sapiens
DEGPAAAGDGLGRPLGPTPSQSR

SEQ ID NO: 115 - protein, 12 aa, Homo Sapiens
DEQHQCSLGNLK

SEQ ID NO: 116 - protein, 20 aa, Homo Sapiens
DFLLQSSTVAAEAQDGPQEA

SEQ ID NO: 117 - protein, 13 aa, Homo Sapiens
DFLPVDPATSNGR

SEQ ID NO: 118 - protein, 13 aa, Homo Sapiens
DFLPVDTSNNNGR

SEQ ID NO: 119 - protein, 12 aa, Homo Sapiens
DFYNPVVPEAQK

SEQ ID NO: 120 - protein, 13 aa, Homo Sapiens
DFYNPVVPEAQKR

SEQ ID NO: 121 - protein, 14 aa, Homo Sapiens
DGEFSVLQLVGMLR

SEQ ID NO: 122 - protein, 16 aa, Homo Sapiens
DGHGTAISNASDVWKK

SEQ ID NO: 123 - protein, 12 aa, Homo Sapiens
DGNGEVTDKPVK

SEQ ID NO: 124 - protein, 11 aa, Homo Sapiens
DGPEFTFTTPK

SEQ ID NO: 125 - protein, 12 aa, Homo Sapiens
DGSFSVVITGLR

SEQ ID NO: 126 - protein, 8 aa, Homo Sapiens
DGVHFKPK

SEQ ID NO: 127 - protein, 16 aa, Homo Sapiens
DHPPIPITDLADNIER

SEQ ID NO: 128 - protein, 33 aa, Homo Sapiens
DHVVVPANTTSVILSGLRPYSSYHLEVQAFNGR

SEQ ID NO: 129 - protein, 16 aa, Homo Sapiens
DINSQQELQNITTDTR

SEQ ID NO: 130 - protein, 7 aa, Homo Sapiens
DIQNQLK

SEQ ID NO: 131 - protein, 11 aa, Homo Sapiens
DIQVASNEILR

SEQ ID NO: 132 - protein, 7 aa, Homo Sapiens
DLAAMDK

SEQ ID NO: 133 - protein, 10 aa, Homo Sapiens
DLAVFLYHLR

SEQ ID NO: 134 - protein, 14 aa, Homo Sapiens
DLLPEDFVVYTYNK

SEQ ID NO: 135 - protein, 10 aa, Homo Sapiens
DLPLLIENMK

SEQ ID NO: 136 - protein, 9 aa, Homo Sapiens
DLPPILLVR

SEQ ID NO: 137 - protein, 10 aa, Homo Sapiens
DLQELGDSDK

SEQ ID NO: 138 - protein, 16 aa, Homo Sapiens
DLQSSVTLDLALDPGR

SEQ ID NO: 139 - protein, 9 aa, Homo Sapiens
DLSQMEALK

SEQ ID NO: 140 - protein, 10 aa, Homo Sapiens
DLSQMEALKR

SEQ ID NO: 141 - protein, 10 aa, Homo Sapiens
DLVEPWVVVR

SEQ ID NO: 142 - protein, 18 aa, Homo Sapiens
DMAGAQAAAVALNEEFLR

SEQ ID NO: 143 - protein, 13 aa, Homo Sapiens
DMEPLTTLEFSEK

SEQ ID NO: 144 - protein, 25 aa, Homo Sapiens
DNIVSYNDEGGGEEDTQAFDIGTLR

SEQ ID NO: 145 - protein, 12 aa, Homo Sapiens
DNIYPAFQMFAK

SEQ ID NO: 146 - protein, 12 aa, Homo Sapiens
DNMLWVEWTTPR

SEQ ID NO: 147 - protein, 11 aa, Homo Sapiens
DNQWLGVTLSR

SEQ ID NO: 148 - protein, 14 aa, Homo Sapiens
DNWISVDSVTSEIK

SEQ ID NO: 149 - protein, 9 aa, Homo Sapiens
DPSVTQVTR

SEQ ID NO: 150 - protein, 7 aa, Homo Sapiens
DQADGSR

SEQ ID NO: 151 - protein, 8 aa, Homo Sapiens
DQFDICAK

SEQ ID NO: 152 - protein, 5 aa, Homo Sapiens
DQLWR

SEQ ID NO: 153 - protein, 10 aa, Homo Sapiens
DQVACLTFFK

SEQ ID NO: 154 - protein, 10 aa, Homo Sapiens
DQVNTVGIPI

SEQ ID NO: 155 - protein, 18 aa, Homo Sapiens
DSFSDPYAIVSFLHQSQK

SEQ ID NO: 156 - protein, 14 aa, Homo Sapiens
DSLLAHSSDPVEMR

SEQ ID NO: 157 - protein, 15 aa, Homo Sapiens
DSLLAHSSDPVEMRR

SEQ ID NO: 158 - protein, 14 aa, Homo Sapiens
DSYLGYSTELALWK

SEQ ID NO: 159 - protein, 14 aa, Homo Sapiens
DTGEIYTTSVTLDR

SEQ ID NO: 160 - protein, 13 aa, Homo Sapiens
DTGELNVTSILDR

SEQ ID NO: 161 - protein, 13 aa, Homo Sapiens
DVIPMADAAGIIR

SEQ ID NO: 162 - protein, 8 aa, Homo Sapiens
DVSAIMNK

SEQ ID NO: 163 - protein, 10 aa, Homo Sapiens
DVVVSVEYSK

SEQ ID NO: 164 - protein, 8 aa, Homo Sapiens
DWLQADMR

SEQ ID NO: 165 - protein, 10 aa, Homo Sapiens
DYVLHMPTLR

SEQ ID NO: 166 - protein, 13 aa, Homo Sapiens
EADLTEETEENLR

SEQ ID NO: 167 - protein, 24 aa, Homo Sapiens
EASVHIQLEGRPSILEMDETSALK

SEQ ID NO: 168 - protein, 21 aa, Homo Sapiens
EDAQINTTIGSVTAQDPDAAR

SEQ ID NO: 169 - protein, 15 aa, Homo Sapiens
EDGYSDASGFGYCFR

SEQ ID NO: 170 - protein, 13 aa, Homo Sapiens
EDTQVDSEARPMK

SEQ ID NO: 171 - protein, 14 aa, Homo Sapiens
EEFVATTESTTETK

SEQ ID NO: 172 - protein, 12 aa, Homo Sapiens
EEGVFTVTPDTK

SEQ ID NO: 173 - protein, 18 aa, Homo Sapiens
EEHEVAVLGAPPSTILPR

SEQ ID NO: 174 - protein, 13 aa, Homo Sapiens
EEHSSYTLTVEAR

SEQ ID NO: 175 - protein, 7 aa, Homo Sapiens
EEIGNLK

SEQ ID NO: 176 - protein, 10 aa, Homo Sapiens
EEINKPPIAK

SEQ ID NO: 177 - protein, 8 aa, Homo Sapiens
EEINQGGR

SEQ ID NO: 178 - protein, 28 aa, Homo Sapiens
EELGVTVYQSPHSGSFTITGNNSNFAQR

SEQ ID NO: 179 - protein, 20 aa, Homo Sapiens
EEMILLANYLDSMYIMLNIR

SEQ ID NO: 180 - protein, 6 aa, Homo Sapiens
EEQAQR

SEQ ID NO: 181 - protein, 5 aa, Homo Sapiens
EERKR

SEQ ID NO: 182 - protein, 16 aa, Homo Sapiens
EETPFFLLTGYALDAR

SEQ ID NO: 183 - protein, 13 aa, Homo Sapiens
EFIYLRPFACDTK

SEQ ID NO: 184 - protein, 19 aa, Homo Sapiens
EFLSEASIMGQFEHPNIIR

SEQ ID NO: 185 - protein, 9 aa, Homo Sapiens
EFNQFAEGK

SEQ ID NO: 186 - protein, 14 aa, Homo Sapiens
EGAQYLMQAAGLGR

SEQ ID NO: 187 - protein, 8 aa, Homo Sapiens
EGEDLSKK

SEQ ID NO: 188 - protein, 16 aa, Homo Sapiens
EGLAHLIQSCGLGGMR

SEQ ID NO: 189 - protein, 9 aa, Homo Sapiens
EGPGEAIVR

SEQ ID NO: 190 - protein, 11 aa, Homo Sapiens
EHALAQAELLK

SEQ ID NO: 191 - protein, 12 aa, Homo Sapiens
EHEEAESGEGTR

SEQ ID NO: 192 - protein, 14 aa, Homo Sapiens
EHEEAESGEGTRRR

SEQ ID NO: 193 - protein, 7 aa, Homo Sapiens
EHEEYLK

SEQ ID NO: 194 - protein, 10 aa, Homo Sapiens
EHQPLPIQWK

SEQ ID NO: 195 - protein, 17 aa, Homo Sapiens
EHSSTANIIVMSLPVAR

SEQ ID NO: 196 - protein, 12 aa, Homo Sapiens
EHSSWDLVGLEK

SEQ ID NO: 197 - protein, 11 aa, Homo Sapiens
EIIDENFAELK

SEQ ID NO: 198 - protein, 14 aa, Homo Sapiens
EIPSHHPTDPVELR

SEQ ID NO: 199 - protein, 15 aa, Homo Sapiens
EIPSHHPTDPVELRR

SEQ ID NO: 200 - protein, 8 aa, Homo Sapiens
EITYQGTR

SEQ ID NO: 201 - protein, 18 aa, Homo Sapiens
EKPAIHHIPGFEVQETSR

SEQ ID NO: 202 - protein, 14 aa, Homo Sapiens
ELAQSQEALQVEQR

SEQ ID NO: 203 - protein, 8 aa, Homo Sapiens
ELLGNNIP

SEQ ID NO: 204 - protein, 16 aa, Homo Sapiens
ELNDIASKPSQEHIFK

SEQ ID NO: 205 - protein, 9 aa, Homo Sapiens
ELNILHEMK

SEQ ID NO: 206 - protein, 10 aa, Homo Sapiens
ELPGELLGYR

SEQ ID NO: 207 - protein, 15 aa, Homo Sapiens
ELTLPVDSTTLDGSK

SEQ ID NO: 208 - protein, 15 aa, Homo Sapiens
ELTYSNFHPLLVSGR

SEQ ID NO: 209 - protein, 13 aa, Homo Sapiens
ELVHIKPDQSNVR

SEQ ID NO: 210 - protein, 25 aa, Homo Sapiens
EMMEEANGQIAPENGTQTPSPPSEK

SEQ ID NO: 211 - protein, 10 aa, Homo Sapiens
EMQNLSQHGR

SEQ ID NO: 212 - protein, 11 aa, Homo Sapiens
EMQNLSQHGRK

SEQ ID NO: 213 - protein, 8 aa, Homo Sapiens
EMSIDQAK

SEQ ID NO: 214 - protein, 13 aa, Homo Sapiens
ENGGASHPLLDQR

SEQ ID NO: 215 - protein, 10 aa, Homo Sapiens
ENPDNLSDFR

SEQ ID NO: 216 - protein, 17 aa, Homo Sapiens
EPFEDGFANGEESTPTR

SEQ ID NO: 217 - protein, 6 aa, Homo Sapiens
EPIDLR

SEQ ID NO: 218 - protein, 7 aa, Homo Sapiens
EPMDQKR

SEQ ID NO: 219 - protein, 19 aa, Homo Sapiens
EQFGQDGPISVHCSAGVGR

SEQ ID NO: 220 - protein, 19 aa, Homo Sapiens
EQFGQDGPITVHCSAGVGR

SEQ ID NO: 221 - protein, 10 aa, Homo Sapiens
EQTPLFLAAR

SEQ ID NO: 222 - protein, 13 aa, Homo Sapiens
ERPPDHQHSAQVK

SEQ ID NO: 223 - protein, 30 aa, Homo Sapiens
ESFLAPSSGVQPTLAMPNIAVGQNVTVTER

SEQ ID NO: 224 - protein, 8 aa, Homo Sapiens
ESHVAMHR

SEQ ID NO: 225 - protein, 12 aa, Homo Sapiens
ESHWIGLTDSER

SEQ ID NO: 226 - protein, 8 aa, Homo Sapiens
ESPAQAPA

SEQ ID NO: 227 - protein, 13 aa, Homo Sapiens
ESSPFLSPLEASR

SEQ ID NO: 228 - protein, 10 aa, Homo Sapiens
ETCVDLHLPR

SEQ ID NO: 229 - protein, 19 aa, Homo Sapiens
ETDSLSAGFVVVMGVDLSR

SEQ ID NO: 230 - protein, 8 aa, Homo Sapiens
ETIEPAVR

SEQ ID NO: 231 - protein, 10 aa, Homo Sapiens
ETLQSEEQQR

SEQ ID NO: 232 - protein, 11 aa, Homo Sapiens
ETLQSEEQQRR

SEQ ID NO: 233 - protein, 7 aa, Homo Sapiens
EVITIYS

SEQ ID NO: 234 - protein, 20 aa, Homo Sapiens
EVLATPSLSASFNAPLLDTK

SEQ ID NO: 235 - protein, 18 aa, Homo Sapiens
EVPGYIVLFYNMSLDVNR

SEQ ID NO: 236 - protein, 8 aa, Homo Sapiens
EVPIYANR

SEQ ID NO: 237 - protein, 10 aa, Homo Sapiens
EVPPAVSDIR

SEQ ID NO: 238 - protein, 7 aa, Homo Sapiens
EVPVAIK

SEQ ID NO: 239 - protein, 23 aa, Homo Sapiens
EVVLLDFAAAGGELGWLTHPYGK

SEQ ID NO: 240 - protein, 7 aa, Homo Sapiens
EWGDGIR

SEQ ID NO: 241 - protein, 11 aa, Homo Sapiens
EYSIEEIEAGR

SEQ ID NO: 242 - protein, 10 aa, Homo Sapiens
FADIVSILDK

SEQ ID NO: 243 - protein, 21 aa, Homo Sapiens
FAPGVTIEEDNCCGCNAIAIR

SEQ ID NO: 244 - protein, 9 aa, Homo Sapiens
FAVPTYAAK

SEQ ID NO: 245 - protein, 19 aa, Homo Sapiens
FCQALGAHLSSFSHVDEIK

SEQ ID NO: 246 - protein, 15 aa, Homo Sapiens
FDLSMPHVQDPSLVR

SEQ ID NO: 247 - protein, 11 aa, Homo Sapiens
FEQEYLNDLMK

SEQ ID NO: 248 - protein, 12 aa, Homo Sapiens
FEQEYLNDLMKK

SEQ ID NO: 249 - protein, 15 aa, Homo Sapiens
FEVIEFDDGAGSVLR

SEQ ID NO: 250 - protein, 8 aa, Homo Sapiens
FFASIGER

SEQ ID NO: 251 - protein, 12 aa, Homo Sapiens
FFPYANGTLGIR

SEQ ID NO: 252 - protein, 15 aa, Homo Sapiens
FGAALTVLGDVNGDK

SEQ ID NO: 253 - protein, 6 aa, Homo Sapiens
FHILFK

SEQ ID NO: 254 - protein, 6 aa, Homo Sapiens
FHLEYR

SEQ ID NO: 255 - protein, 18 aa, Homo Sapiens
FIKPWESPDEMELDELLK

SEQ ID NO: 256 - protein, 6 aa, Homo Sapiens
FLCEVK

SEQ ID NO: 257 - protein, 8 aa, Homo Sapiens
FLDDLGLK

SEQ ID NO: 258 - protein, 18 aa, Homo Sapiens
FLEENSSDPTYTSSLGGK

SEQ ID NO: 259 - protein, 8 aa, Homo Sapiens
FLNLTSEK

SEQ ID NO: 260 - protein, 9 aa, Homo Sapiens
FLPGGTLCR

SEQ ID NO: 261 - protein, 10 aa, Homo Sapiens
FMFDLFQQFR

SEQ ID NO: 262 - protein, 11 aa, Homo Sapiens
FMFDLFQQFRK

SEQ ID NO: 263 - protein, 10 aa, Homo Sapiens
FPQVVSALDK

SEQ ID NO: 264 - protein, 7 aa, Homo Sapiens
FPSGTLR

SEQ ID NO: 265 - protein, 15 aa, Homo Sapiens
FPVTFGEECLYMSAK

SEQ ID NO: 266 - protein, 6 aa, Homo Sapiens
FQGIYR

SEQ ID NO: 267 - protein, 6 aa, Homo Sapiens
FQLAAR

SEQ ID NO: 268 - protein, 13 aa, Homo Sapiens
FQLSNSGPNSTIK

SEQ ID NO: 269 - protein, 11 aa, Homo Sapiens
FQTHFTFEEFR

SEQ ID NO: 270 - protein, 12 aa, Homo Sapiens
FQTHFTFEEFRR

SEQ ID NO: 271 - protein, 12 aa, Homo Sapiens
FQVDLVSENAGR

SEQ ID NO: 272 - protein, 7 aa, Homo Sapiens
FSDVTGK

SEQ ID NO: 273 - protein, 16 aa, Homo Sapiens
FSFDDFLGSLQLDLNR

SEQ ID NO: 274 - protein, 6 aa, Homo Sapiens
FSSYEK

SEQ ID NO: 275 - protein, 15 aa, Homo Sapiens
FTLTGLKPDTTYDIK

SEQ ID NO: 276 - protein, 11 aa, Homo Sapiens
FTMLECLSLPR

SEQ ID NO: 277 - protein, 7 aa, Homo Sapiens
FTQDTFR

SEQ ID NO: 278 - protein, 12 aa, Homo Sapiens
FTTEIHPSCVTR

SEQ ID NO: 279 - protein, 9 aa, Homo Sapiens
FVWNGHLLR

SEQ ID NO: 280 - protein, 19 aa, Homo Sapiens
FYQTSVESTDFANAPEESR

SEQ ID NO: 281 - protein, 21 aa, Homo Sapiens
FYQTSVESTDFANAPEESRKK

SEQ ID NO: 282 - protein, 11 aa, Homo Sapiens
GAEAFEIALPR

SEQ ID NO: 283 - protein, 9 aa, Homo Sapiens
GALADNLLR

SEQ ID NO: 284 - protein, 21 aa, Homo Sapiens
GALILSNVQPSDTMVTQCEAR

SEQ ID NO: 285 - protein, 13 aa, Homo Sapiens
GANTHLSTFSFTK

SEQ ID NO: 286 - protein, 12 aa, Homo Sapiens
GAPCTTPPSAPR

SEQ ID NO: 287 - protein, 21 aa, Homo Sapiens
GAVYLFHGVLGPSISPSHSQR

SEQ ID NO: 288 - protein, 10 aa, Homo Sapiens
GDSNSYNVRR

SEQ ID NO: 289 - protein, 17 aa, Homo Sapiens
GEGNETTNMVITWKPLR

SEQ ID NO: 290 - protein, 14 aa, Homo Sapiens
GEGPEAGAGGAGGR

SEQ ID NO: 291 - protein, 7 aa, Homo Sapiens
GEGVAYR

SEQ ID NO: 292 - protein, 12 aa, Homo Sapiens
GEPGPPGPAGER

SEQ ID NO: 293 - protein, 15 aa, Homo Sapiens
GETYTYDWQLITHPR

SEQ ID NO: 294 - protein, 6 aa, Homo Sapiens
GFFGYK

SEQ ID NO: 295 - protein, 12 aa, Homo Sapiens
GFPTIDMGPQLK

SEQ ID NO: 296 - protein, 16 aa, Homo Sapiens
GGAQITFLATFDVSPK

SEQ ID NO: 297 - protein, 16 aa, Homo Sapiens
GGCITLISSEGYVSSK

SEQ ID NO: 298 - protein, 10 aa, Homo Sapiens
GGGAYYLISR

SEQ ID NO: 299 - protein, 11 aa, Homo Sapiens
GGQVSVCPLPR

SEQ ID NO: 300 - protein, 7 aa, Homo Sapiens
GIDYYDR

SEQ ID NO: 301 - protein, 14 aa, Homo Sapiens
GITEPPFGIFVFNK

SEQ ID NO: 302 - protein, 16 aa, Homo Sapiens
GLPSLTSVSWNISVPR

SEQ ID NO: 303 - protein, 9 aa, Homo Sapiens
GNLSFGWVR

SEQ ID NO: 304 - protein, 11 aa, Homo Sapiens
GNNVEKPLELR

SEQ ID NO: 305 - protein, 10 aa, Homo Sapiens
GNSAGGLQHR

SEQ ID NO: 306 - protein, 12 aa, Homo Sapiens
GPGPYSPSVQFR

SEQ ID NO: 307 - protein, 12 aa, Homo Sapiens
GPIVPLNVADQK

SEQ ID NO: 308 - protein, 8 aa, Homo Sapiens
GPPSEAVR

SEQ ID NO: 309 - protein, 21 aa, Homo Sapiens
GPPSEPVLTQTSEQAPSSAPR

SEQ ID NO: 310 - protein, 10 aa, Homo Sapiens
GPRPNPAIMR

SEQ ID NO: 311 - protein, 21 aa, Homo Sapiens
GQIIGNFQAFDEDTGLPAHAR

SEQ ID NO: 312 - protein, 8 aa, Homo Sapiens
GQLSFNLR

SEQ ID NO: 313 - protein, 17 aa, Homo Sapiens
GQVVTVHLQTSLENGTR

SEQ ID NO: 314 - protein, 23 aa, Homo Sapiens
GSEKPLEQTFATMVSSLGSGMMR

SEQ ID NO: 315 - protein, 12 aa, Homo Sapiens
GSGPLSPSIQSR

SEQ ID NO: 316 - protein, 14 aa, Homo Sapiens
GSIPVFWSQRPNLK

SEQ ID NO: 317 - protein, 23 aa, Homo Sapiens
GSPGKPGPQGSSGDPGPPGPPGK

SEQ ID NO: 318 - protein, 25 aa, Homo Sapiens
GSPQLDCGPPALQEMPINQGGEGKK

SEQ ID NO: 319 - protein, 12 aa, Homo Sapiens
GSQGPPGPTGNK

SEQ ID NO: 320 - protein, 17 aa, Homo Sapiens
GSQPSGELLASFELIQR

SEQ ID NO: 321 - protein, 14 aa, Homo Sapiens
GSSAGGLQHLVSIR

SEQ ID NO: 322 - protein, 9 aa, Homo Sapiens
GSSSASIVK

SEQ ID NO: 323 - protein, 18 aa, Homo Sapiens
GSVTFHCALGPEVANVAK

SEQ ID NO: 324 - protein, 7 aa, Homo Sapiens
GTTYIFR

SEQ ID NO: 325 - protein, 17 aa, Homo Sapiens
GVEGSDYINASFIDGYR

SEQ ID NO: 326 - protein, 17 aa, Homo Sapiens
GVEGSDYINASFLDGYR

SEQ ID NO: 327 - protein, 11 aa, Homo Sapiens
GVQSLVLGAPR

SEQ ID NO: 328 - protein, 8 aa, Homo Sapiens
GWHFYDDR

SEQ ID NO: 329 - protein, 10 aa, Homo Sapiens
GWMIGFEEHK

SEQ ID NO: 330 - protein, 8 aa, Homo Sapiens
GYNVTYWR

SEQ ID NO: 331 - protein, 8 aa, Homo Sapiens
GYQVTYVR

SEQ ID NO: 332 - protein, 11 aa, Homo Sapiens
HFDSQVIIYGK

SEQ ID NO: 333 - protein, 8 aa, Homo Sapiens
HFVSLCQK

SEQ ID NO: 334 - protein, 12 aa, Homo Sapiens
HGLPSADAPSLK

SEQ ID NO: 335 - protein, 11 aa, Homo Sapiens
HGQYLIGHGTK

SEQ ID NO: 336 - protein, 11 aa, Homo Sapiens
HIWPNVPDPSK

SEQ ID NO: 337 - protein, 9 aa, Homo Sapiens
HLPETEAGR

SEQ ID NO: 338 - protein, 12 aa, Homo Sapiens
HMATTQDEVHTK

SEQ ID NO: 339 - protein, 12 aa, Homo Sapiens
HMWPFICQFIEK

SEQ ID NO: 340 - protein, 13 aa, Homo Sapiens
HNSVVLGWPYGWR

SEQ ID NO: 341 - protein, 23 aa, Homo Sapiens
HNTDAGLLTTVGSLLPGITYSLR

SEQ ID NO: 342 - protein, 6 aa, Homo Sapiens
HQMAVK

SEQ ID NO: 343 - protein, 6 aa, Homo Sapiens
HQVVHK

SEQ ID NO: 344 - protein, 14 aa, Homo Sapiens
HSHTNLSISTGVTK

SEQ ID NO: 345 - protein, 14 aa, Homo Sapiens
HVEMYQWVETEESR

SEQ ID NO: 346 - protein, 9 aa, Homo Sapiens
HVSPVTPPR

SEQ ID NO: 347 - protein, 8 aa, Homo Sapiens
HVVDGISR

SEQ ID NO: 348 - protein, 7 aa, Homo Sapiens
HWVPAEK

SEQ ID NO: 349 - protein, 8 aa, Homo Sapiens
IADFGLAR

SEQ ID NO: 350 - protein, 9 aa, Homo Sapiens
IAGSQLSSR

SEQ ID NO: 351 - protein, 10 aa, Homo Sapiens
IAPCYQDYVK

SEQ ID NO: 352 - protein, 14 aa, Homo Sapiens
IAPPASDFLAHIQK

SEQ ID NO: 353 - protein, 7 aa, Homo Sapiens
IDATVVR

SEQ ID NO: 354 - protein, 17 aa, Homo Sapiens
IDFSDIMVLGDINTKPK

SEQ ID NO: 355 - protein, 18 aa, Homo Sapiens
IDFSDIMVLGDINTKPKK

SEQ ID NO: 356 - protein, 6 aa, Homo Sapiens
IDHDRR

SEQ ID NO: 357 - protein, 5 aa, Homo Sapiens
IDHYR

SEQ ID NO: 358 - protein, 13 aa, Homo Sapiens
IDSLEDQIEEFSK

SEQ ID NO: 359 - protein, 18 aa, Homo Sapiens
IDTIAPDEITVSSDFEAR

SEQ ID NO: 360 - protein, 15 aa, Homo Sapiens
IEEVIGAGEFGEVCR

SEQ ID NO: 361 - protein, 8 aa, Homo Sapiens
IEGLQISK

SEQ ID NO: 362 - protein, 7 aa, Homo Sapiens
IEMVDYK

SEQ ID NO: 363 - protein, 13 aa, Homo Sapiens
IETQNQLLGIADR

SEQ ID NO: 364 - protein, 15 aa, Homo Sapiens
IFTVAQMDDNSIQMK

SEQ ID NO: 365 - protein, 16 aa, Homo Sapiens
IFTVAQMDDNSIQMKK

SEQ ID NO: 366 - protein, 11 aa, Homo Sapiens
IGDDNENLTFK

SEQ ID NO: 367 - protein, 11 aa, Homo Sapiens
IGETVVDLENR

SEQ ID NO: 368 - protein, 12 aa, Homo Sapiens
IHFIEAQDLQGK

SEQ ID NO: 369 - protein, 9 aa, Homo Sapiens
IHSDLAEER

SEQ ID NO: 370 - protein, 6 aa, Homo Sapiens
IIDWDR

SEQ ID NO: 371 - protein, 9 aa, Homo Sapiens
IIEGEPNLK

SEQ ID NO: 372 - protein, 19 aa, Homo Sapiens
IIMYELVYWAAEDEDQQHK

SEQ ID NO: 373 - protein, 9 aa, Homo Sapiens
ILASVQHMK

SEQ ID NO: 374 - protein, 17 aa, Homo Sapiens
ILDATDQESLELKPTSR

SEQ ID NO: 375 - protein, 17 aa, Homo Sapiens
ILDESEDTDLPYPPPQR

SEQ ID NO: 376 - protein, 7 aa, Homo Sapiens
ILELEEK

SEQ ID NO: 377 - protein, 8 aa, Homo Sapiens
ILLNPQDK

SEQ ID NO: 378 - protein, 16 aa, Homo Sapiens
ILYNGQSVEVDGHSMR

SEQ ID NO: 379 - protein, 17 aa, Homo Sapiens
ILYNGQSVEVDGHSMRK

SEQ ID NO: 380 - protein, 11 aa, Homo Sapiens
IMFVDPSLTVR

SEQ ID NO: 381 - protein, 15 aa, Homo Sapiens
INATDADEPNTLNSK

SEQ ID NO: 382 - protein, 11 aa, Homo Sapiens
INGIPVEELAK

SEQ ID NO: 383 - protein, 7 aa, Homo Sapiens
INHLIFR

SEQ ID NO: 384 - protein, 12 aa, Homo Sapiens
INSWVESQTNEK

SEQ ID NO: 385 - protein, 11 aa, Homo Sapiens
IPENFFEEESR

SEQ ID NO: 386 - protein, 12 aa, Homo Sapiens
IPNPHLGPVEER

SEQ ID NO: 387 - protein, 17 aa, Homo Sapiens
IQDLLAEGTITGVIDDR

SEQ ID NO: 388 - protein, 12 aa, Homo Sapiens
IQLSWLLPPQER

SEQ ID NO: 389 - protein, 7 aa, Homo Sapiens
IQMTWTR

SEQ ID NO: 390 - protein, 15 aa, Homo Sapiens
IQNSSCTSLEHCFRK

SEQ ID NO: 391 - protein, 10 aa, Homo Sapiens
IQPYTEQSTK

SEQ ID NO: 392 - protein, 14 aa, Homo Sapiens
ISEWPIDDHFTYSR

SEQ ID NO: 393 - protein, 16 aa, Homo Sapiens
ISSNPNPVVQMSVGHK

SEQ ID NO: 394 - protein, 14 aa, Homo Sapiens
ISVTVSETFDPEEK

SEQ ID NO: 395 - protein, 10 aa, Homo Sapiens
ITDNELELYK

SEQ ID NO: 396 - protein, 15 aa, Homo Sapiens
ITQLGQSAEDLQGSR

SEQ ID NO: 397 - protein, 16 aa, Homo Sapiens
ITQLGQSAEDLQGSRR

SEQ ID NO: 398 - protein, 15 aa, Homo Sapiens
ITVPLVSEVQIAQLR

SEQ ID NO: 399 - protein, 6 aa, Homo Sapiens
ITWMKK

SEQ ID NO: 400 - protein, 10 aa, Homo Sapiens
IVAISEDYPR

SEQ ID NO: 401 - protein, 9 aa, Homo Sapiens
IVFTPTICK

SEQ ID NO: 402 - protein, 14 aa, Homo Sapiens
IVVEDVDEPPVFSK

SEQ ID NO: 403 - protein, 15 aa, Homo Sapiens
IYPLPEDPAIPMPPR

SEQ ID NO: 404 - protein, 10 aa, Homo Sapiens
IYVVDLSNER

SEQ ID NO: 405 - protein, 7 aa, Homo Sapiens
KAAELDR

SEQ ID NO: 406 - protein, 8 aa, Homo Sapiens
KAAELDRR

SEQ ID NO: 407 - protein, 7 aa, Homo Sapiens
KAESPPR

SEQ ID NO: 408 - protein, 9 aa, Homo Sapiens
KDWLQADMR

SEQ ID NO: 409 - protein, 9 aa, Homo Sapiens
KEGDSLDYK

SEQ ID NO: 410 - protein, 15 aa, Homo Sapiens
KENIIAFEEIIEPYR

SEQ ID NO: 411 - protein, 8 aa, Homo Sapiens
KENITYMK

SEQ ID NO: 412 - protein, 9 aa, Homo Sapiens
KENITYMKR

SEQ ID NO: 413 - protein, 8 aa, Homo Sapiens
KESCVAIK

SEQ ID NO: 414 - protein, 8 aa, Homo Sapiens
KEVPVAIK

SEQ ID NO: 415 - protein, 13 aa, Homo Sapiens
KFVPGCFVCLESR

SEQ ID NO: 416 - protein, 21 aa, Homo Sapiens
KFYQTSVESTDFANAPEESRK

SEQ ID NO: 417 - protein, 10 aa, Homo Sapiens
KGDSNSYNVR

SEQ ID NO: 418 - protein, 11 aa, Homo Sapiens
KGDSNSYNVRR

SEQ ID NO: 419 - protein, 18 aa, Homo Sapiens
KGFGGTAGMAFVGTVCSR

SEQ ID NO: 420 - protein, 13 aa, Homo Sapiens
KINSWVESQTNEK

SEQ ID NO: 421 - protein, 9 aa, Homo Sapiens
KKENITYMK

SEQ ID NO: 422 - protein, 11 aa, Homo Sapiens
KKGDSNSYNVR

SEQ ID NO: 423 - protein, 8 aa, Homo Sapiens
KLGDQTGK

SEQ ID NO: 424 - protein, 19 aa, Homo Sapiens
KLIADLQPNTEYSFVLMNR

SEQ ID NO: 425 - protein, 8 aa, Homo Sapiens
KLPQGESR

SEQ ID NO: 426 - protein, 14 aa, Homo Sapiens
KLPSRPPPHYPGIK

SEQ ID NO: 427 - protein, 8 aa, Homo Sapiens
KMLGNPSR

SEQ ID NO: 428 - protein, 6 aa, Homo Sapiens
KNGYNR

SEQ ID NO: 429 - protein, 15 aa, Homo Sapiens
KNLVDPFVEVSFAGK

SEQ ID NO: 430 - protein, 18 aa, Homo Sapiens
KNPNFDICTLFMEVMLPR

SEQ ID NO: 431 - protein, 8 aa, Homo Sapiens
KPFPEDLK

SEQ ID NO: 432 - protein, 19 aa, Homo Sapiens
KPLMVIHHLEDCQYSQALK

SEQ ID NO: 433 - protein, 21 aa, Homo Sapiens
KQNAYIATQGPLPETMGDFWR

SEQ ID NO: 434 - protein, 8 aa, Homo Sapiens
KRAESDSR

SEQ ID NO: 435 - protein, 18 aa, Homo Sapiens
KSDLDTSKPLSEKPITHK

SEQ ID NO: 436 - protein, 7 aa, Homo Sapiens
KTDAFRR

SEQ ID NO: 437 - protein, 6 aa, Homo Sapiens
KTEEKK

SEQ ID NO: 438 - protein, 15 aa, Homo Sapiens
KTMLHLTDIQLQDNK

SEQ ID NO: 439 - protein, 10 aa, Homo Sapiens
KVECEHGFGR

SEQ ID NO: 440 - protein, 16 aa, Homo Sapiens
KVFAQNEEIQEMAQNK

SEQ ID NO: 441 - protein, 7 aa, Homo Sapiens
KVHGLYR

SEQ ID NO: 442 - protein, 8 aa, Homo Sapiens
KYFWTGLR

SEQ ID NO: 443 - protein, 5 aa, Homo Sapiens
KYWCR

SEQ ID NO: 444 - protein, 9 aa, Homo Sapiens
LATEEPPPR

SEQ ID NO: 445 - protein, 16 aa, Homo Sapiens
LDCQVQGRPQPEVTWR

SEQ ID NO: 446 - protein, 8 aa, Homo Sapiens
LDEDLHVK

SEQ ID NO: 447 - protein, 5 aa, Homo Sapiens
LDHYR

SEQ ID NO: 448 - protein, 16 aa, Homo Sapiens
LDTEVANLSVIMEEMK

SEQ ID NO: 449 - protein, 10 aa, Homo Sapiens
LEDPHVDIIR

SEQ ID NO: 450 - protein, 6 aa, Homo Sapiens
LEEEQK

SEQ ID NO: 451 - protein, 9 aa, Homo Sapiens
LEEGPPHTK

SEQ ID NO: 452 - protein, 11 aa, Homo Sapiens
LEEGQATSWSR

SEQ ID NO: 453 - protein, 9 aa, Homo Sapiens
LEEQDEFEK

SEQ ID NO: 454 - protein, 7 aa, Homo Sapiens
LEGVISK

SEQ ID NO: 455 - protein, 7 aa, Homo Sapiens
LENGEPR

SEQ ID NO: 456 - protein, 7 aa, Homo Sapiens
LETADLK

SEQ ID NO: 457 - protein, 15 aa, Homo Sapiens
LEWLTLMPNASNLDK

SEQ ID NO: 458 - protein, 9 aa, Homo Sapiens
LFHASYGAR

SEQ ID NO: 459 - protein, 10 aa, Homo Sapiens
LFHASYGARR

SEQ ID NO: 460 - protein, 8 aa, Homo Sapiens
LFHLHSQK

SEQ ID NO: 461 - protein, 8 aa, Homo Sapiens
LGDQTGKK

SEQ ID NO: 462 - protein, 18 aa, Homo Sapiens
LGQVPPGESVTAMELEFK

SEQ ID NO: 463 - protein, 10 aa, Homo Sapiens
LGTAMSHEIR

SEQ ID NO: 464 - protein, 9 aa, Homo Sapiens
LGVLHVGQK

SEQ ID NO: 465 - protein, 6 aa, Homo Sapiens
LHEDDK

SEQ ID NO: 466 - protein, 7 aa, Homo Sapiens
LHITPEK

SEQ ID NO: 467 - protein, 18 aa, Homo Sapiens
LIADLQPNTEYSFVLMNR

SEQ ID NO: 468 - protein, 10 aa, Homo Sapiens
LLQEAYMTPK

SEQ ID NO: 469 - protein, 12 aa, Homo Sapiens
LLSDKPQDFQIR

SEQ ID NO: 470 - protein, 11 aa, Homo Sapiens
LMEWTSLQNMR

SEQ ID NO: 471 - protein, 6 aa, Homo Sapiens
LNELLK

SEQ ID NO: 472 - protein, 8 aa, Homo Sapiens
LNEVIVTR

SEQ ID NO: 473 - protein, 19 aa, Homo Sapiens
LNGSSLHLEWSAPLESGGR

SEQ ID NO: 474 - protein, 6 aa, Homo Sapiens
LNVEER

SEQ ID NO: 475 - protein, 9 aa, Homo Sapiens
LNYQTPGMR

SEQ ID NO: 476 - protein, 7 aa, Homo Sapiens
LPDFESR

SEQ ID NO: 477 - protein, 20 aa, Homo Sapiens
LPGGQWIYMSDNYTDVNGEK

SEQ ID NO: 478 - protein, 7 aa, Homo Sapiens
LPGHQKR

SEQ ID NO: 479 - protein, 21 aa, Homo Sapiens
LPPPPDCPTSLHQLMLDCWQK

SEQ ID NO: 480 - protein, 7 aa, Homo Sapiens
LPQGESR

SEQ ID NO: 481 - protein, 13 aa, Homo Sapiens
LPSTSGSEGVPFR

SEQ ID NO: 482 - protein, 14 aa, Homo Sapiens
LPTGGCYGVPPDLR

SEQ ID NO: 483 - protein, 23 aa, Homo Sapiens
LPTPMDCPSAIYQLMMQCWQQER

SEQ ID NO: 484 - protein, 9 aa, Homo Sapiens
LPVGLYFIK

SEQ ID NO: 485 - protein, 11 aa, Homo Sapiens
LQASGDALVDR

SEQ ID NO: 486 - protein, 22 aa, Homo Sapiens
LSLLEEPGNGTFTVILNQLTSR

SEQ ID NO: 487 - protein, 10 aa, Homo Sapiens
LSLVLVPAQK

SEQ ID NO: 488 - protein, 16 aa, Homo Sapiens
LSNTSPEFQEMSLLER

SEQ ID NO: 489 - protein, 10 aa, Homo Sapiens
LSPYVHYTFR

SEQ ID NO: 490 - protein, 10 aa, Homo Sapiens
LSQCHELWER

SEQ ID NO: 491 - protein, 13 aa, Homo Sapiens
LSVFAETYENETK

SEQ ID NO: 492 - protein, 23 aa, Homo Sapiens
LSVLEEEQLPPGFPSIDMGPQLK

SEQ ID NO: 493 - protein, 11 aa, Homo Sapiens
LSWYDPDFQAR

SEQ ID NO: 494 - protein, 13 aa, Homo Sapiens
LTADLSLDTLDAR

SEQ ID NO: 495 - protein, 9 aa, Homo Sapiens
LTAVESDLK

SEQ ID NO: 496 - protein, 15 aa, Homo Sapiens
LTDVVIGAPGEEENR

SEQ ID NO: 497 - protein, 9 aa, Homo Sapiens
LTPGLYEFK

SEQ ID NO: 498 - protein, 18 aa, Homo Sapiens
LTQIETGENVTGMELEFK

SEQ ID NO: 499 - protein, 6 aa, Homo Sapiens
LTYSSR

SEQ ID NO: 500 - protein, 9 aa, Homo Sapiens
LVHIIGALR

SEQ ID NO: 501 - protein, 11 aa, Homo Sapiens
LVLLNMPGPPR

SEQ ID NO: 502 - protein, 15 aa, Homo Sapiens
LVLSDLHLLTQSQVR

SEQ ID NO: 503 - protein, 11 aa, Homo Sapiens
LVNIMPYESTR

SEQ ID NO: 504 - protein, 12 aa, Homo Sapiens
LVSYTNLTQGAK

SEQ ID NO: 505 - protein, 7 aa, Homo Sapiens
LWMNVEK

SEQ ID NO: 506 - protein, 7 aa, Homo Sapiens
LYTYEPR

SEQ ID NO: 507 - protein, 6 aa, Homo Sapiens
MAFDFR

SEQ ID NO: 508 - protein, 11 aa, Homo Sapiens
MALHLPWFHPR

SEQ ID NO: 509 - protein, 9 aa, Homo Sapiens
MAPEPAPGR

SEQ ID NO: 510 - protein, 7 aa, Homo Sapiens
MDILEER

SEQ ID NO: 511 - protein, 8 aa, Homo Sapiens
MDVGTIYR

SEQ ID NO: 512 - protein, 7 aa, Homo Sapiens
MELQAAR

SEQ ID NO: 513 - protein, 6 aa, Homo Sapiens
MEPLEK

SEQ ID NO: 514 - protein, 9 aa, Homo Sapiens
MESEELADR

SEQ ID NO: 515 - protein, 12 aa, Homo Sapiens
MFELTCTLPLEK

SEQ ID NO: 516 - protein, 17 aa, Homo Sapiens
MGQPPAEEAEQPGALAR

SEQ ID NO: 517 - protein, 6 aa, Homo Sapiens
MHTAVK

SEQ ID NO: 518 - protein, 22 aa, Homo Sapiens
MLSASTMLVQWEPPEEPNGLVR

SEQ ID NO: 519 - protein, 15 aa, Homo Sapiens
MLWEHNSTIIVMLTK

SEQ ID NO: 520 - protein, 15 aa, Homo Sapiens
MLWEHNSTIVVMLTK

SEQ ID NO: 521 - protein, 13 aa, Homo Sapiens
MPHFTVVPVDGPR

SEQ ID NO: 522 - protein, 26 aa, Homo Sapiens
MPNVPNTQPAIMKPTEEHPAYTQIAK

SEQ ID NO: 523 - protein, 18 aa, Homo Sapiens
MQQYTEHFMAAGYTAIEK

SEQ ID NO: 524 - protein, 22 aa, Homo Sapiens
MSAGGASVPPPPNPAVSFPPPR

SEQ ID NO: 525 - protein, 11 aa, Homo Sapiens
MSDVSTSVQSK

SEQ ID NO: 526 - protein, 16 aa, Homo Sapiens
MTTSGALFPSLVPGSR

SEQ ID NO: 527 - protein, 8 aa, Homo Sapiens
MVEEVDGR

SEQ ID NO: 528 - protein, 15 aa, Homo Sapiens
MVFFVQNEPPHQIFK

SEQ ID NO: 529 - protein, 6 aa, Homo Sapiens
MVWEQR

SEQ ID NO: 530 - protein, 16 aa, Homo Sapiens
MYPETFLYVTSSAGIR

SEQ ID NO: 531 - protein, 6 aa, Homo Sapiens
MYYTHR

SEQ ID NO: 532 - protein, 7 aa, Homo Sapiens
MYYTHRR

SEQ ID NO: 533 - protein, 8 aa, Homo Sapiens
MYYTHRRR

SEQ ID NO: 534 - protein, 14 aa, Homo Sapiens
NDFQNLQQVFLQAK

SEQ ID NO: 535 - protein, 10 aa, Homo Sapiens
NFATMMNFVR

SEQ ID NO: 536 - protein, 8 aa, Homo Sapiens
NGVGGMAK

SEQ ID NO: 537 - protein, 19 aa, Homo Sapiens
NGVITQYSVAYEAVDGEDR

SEQ ID NO: 538 - protein, 10 aa, Homo Sapiens
NGVSGLVTSR

SEQ ID NO: 539 - protein, 11 aa, Homo Sapiens
NIAFYPSNHER

SEQ ID NO: 540 - protein, 6 aa, Homo Sapiens
NIFYIK

SEQ ID NO: 541 - protein, 14 aa, Homo Sapiens
NLALFEEELDIRPK

SEQ ID NO: 542 - protein, 15 aa, Homo Sapiens
NLIAFSEDGSDPYVR

SEQ ID NO: 543 - protein, 8 aa, Homo Sapiens
NLITNLQR

SEQ ID NO: 544 - protein, 13 aa, Homo Sapiens
NLQSQSSVNVIVK

SEQ ID NO: 545 - protein, 12 aa, Homo Sapiens
NLRPMLAADAQR

SEQ ID NO: 546 - protein, 11 aa, Homo Sapiens
NLSPDGQYVPR

SEQ ID NO: 547 - protein, 14 aa, Homo Sapiens
NLVDPFVEVSFAGK

SEQ ID NO: 548 - protein, 19 aa, Homo Sapiens
NMYLTGLCFLLGPTQLTQR

SEQ ID NO: 549 - protein, 6 aa, Homo Sapiens
NNEPLR

SEQ ID NO: 550 - protein, 8 aa, Homo Sapiens
NNFSDGFR

SEQ ID NO: 551 - protein, 17 aa, Homo Sapiens
NPNFDICTLFMEVMLPR

SEQ ID NO: 552 - protein, 13 aa, Homo Sapiens
NPVLDCSIAGCLR

SEQ ID NO: 553 - protein, 21 aa, Homo Sapiens
NSEGDENYMEFLEVLTEGLER

SEQ ID NO: 554 - protein, 6 aa, Homo Sapiens
NSYCKK

SEQ ID NO: 555 - protein, 15 aa, Homo Sapiens
NVGLMICGHVHMGPR

SEQ ID NO: 556 - protein, 16 aa, Homo Sapiens
NVGLMICGHVHMGPRR

SEQ ID NO: 557 - protein, 9 aa, Homo Sapiens
NVHLEFTER

SEQ ID NO: 558 - protein, 10 aa, Homo Sapiens
NVLELSNVVR

SEQ ID NO: 559 - protein, 8 aa, Homo Sapiens
NVPDDVLR

SEQ ID NO: 560 - protein, 16 aa, Homo Sapiens
NVPPGLDEYNPFSDSR

SEQ ID NO: 561 - protein, 10 aa, Homo Sapiens
NVSGFSIANR

SEQ ID NO: 562 - protein, 22 aa, Homo Sapiens
NVSVQPEISEGLATTPSTQQVK

SEQ ID NO: 563 - protein, 7 aa, Homo Sapiens
NWEEAER

SEQ ID NO: 564 - protein, 7 aa, Homo Sapiens
NYTIFYR

SEQ ID NO: 565 - protein, 6 aa, Homo Sapiens
QCLIKK

SEQ ID NO: 566 - protein, 6 aa, Homo Sapiens
QEELER

SEQ ID NO: 567 - protein, 18 aa, Homo Sapiens
QEQDIVFLIDGSGSISSR

SEQ ID NO: 568 - protein, 13 aa, Homo Sapiens
QESTSVLLQQSEK

SEQ ID NO: 569 - protein, 14 aa, Homo Sapiens
QESTSVLLQQSEKK

SEQ ID NO: 570 - protein, 16 aa, Homo Sapiens
QFHQLAAQGPQECLVR

SEQ ID NO: 571 - protein, 24 aa, Homo Sapiens
QFQFMAWPDHGVPEYPTPILAFLR

SEQ ID NO: 572 - protein, 14 aa, Homo Sapiens
QFQFTDWPEQGVPK

SEQ ID NO: 573 - protein, 15 aa, Homo Sapiens
QGAQWAALCDSSSAR

SEQ ID NO: 574 - protein, 18 aa, Homo Sapiens
QGHFYGETAAVYVAVEER

SEQ ID NO: 575 - protein, 19 aa, Homo Sapiens
QGHFYGETAAVYVAVEERK

SEQ ID NO: 576 - protein, 12 aa, Homo Sapiens
QGLTVSFIPYFK

SEQ ID NO: 577 - protein, 9 aa, Homo Sapiens
QGPVGSGRR

SEQ ID NO: 578 - protein, 6 aa, Homo Sapiens
QHGQIR

SEQ ID NO: 579 - protein, 9 aa, Homo Sapiens
QIFGFESNK

SEQ ID NO: 580 - protein, 19 aa, Homo Sapiens
QIGPGESCPDGVTHSISGR

SEQ ID NO: 581 - protein, 20 aa, Homo Sapiens
QNAYIATQGPLPETMGDFWR

SEQ ID NO: 582 - protein, 17 aa, Homo Sapiens
QNCSQHESSPDISHFER

SEQ ID NO: 583 - protein, 27 aa, Homo Sapiens
QPGNMAGNFNLSLQGCDQDAQSPGILR

SEQ ID NO: 584 - protein, 7 aa, Homo Sapiens
QPGSVPR

SEQ ID NO: 585 - protein, 15 aa, Homo Sapiens
QPHYSAFGSVGEWLR

SEQ ID NO: 586 - protein, 15 aa, Homo Sapiens
QPQQFPSRPPPPQPK

SEQ ID NO: 587 - protein, 10 aa, Homo Sapiens
QSPEDVYFSK

SEQ ID NO: 588 - protein, 16 aa, Homo Sapiens
QSSGENCDVVVNTLGK

SEQ ID NO: 589 - protein, 17 aa, Homo Sapiens
QSSGENCDVVVNTLGKR

SEQ ID NO: 590 - protein, 10 aa, Homo Sapiens
QVIINLINQK

SEQ ID NO: 591 - protein, 12 aa, Homo Sapiens
QVSSVNEEDFVR

SEQ ID NO: 592 - protein, 10 aa, Homo Sapiens
QWSNVFNILR

SEQ ID NO: 593 - protein, 9 aa, Homo Sapiens
QYAGTGALK

SEQ ID NO: 594 - protein, 16 aa, Homo Sapiens
QYNSFNFALLEDVQAK

SEQ ID NO: 595 - protein, 8 aa, Homo Sapiens
RAESDSRK

SEQ ID NO: 596 - protein, 9 aa, Homo Sapiens
RAMATLLSK

SEQ ID NO: 597 - protein, 8 aa, Homo Sapiens
RAPAFEGR

SEQ ID NO: 598 - protein, 6 aa, Homo Sapiens
RDLGSR

SEQ ID NO: 599 - protein, 10 aa, Homo Sapiens
RDLSQMEALK

SEQ ID NO: 600 - protein, 6 aa, Homo Sapiens
RDQLWR

SEQ ID NO: 601 - protein, 9 aa, Homo Sapiens
REITYQGTR

SEQ ID NO: 602 - protein, 9 aa, Homo Sapiens
REQLTETDK

SEQ ID NO: 603 - protein, 12 aa, Homo Sapiens
RGDFFYHSENPK

SEQ ID NO: 604 - protein, 8 aa, Homo Sapiens
RHGETCYK

SEQ ID NO: 605 - protein, 5 aa, Homo Sapiens
RKNQR

SEQ ID NO: 606 - protein, 10 aa, Homo Sapiens
RLNYQTPGMR

SEQ ID NO: 607 - protein, 7 aa, Homo Sapiens
RMEPLEK

SEQ ID NO: 608 - protein, 14 aa, Homo Sapiens
RPDTSFLWFTSPYK

SEQ ID NO: 609 - protein, 8 aa, Homo Sapiens
RPPNAWHK

SEQ ID NO: 610 - protein, 8 aa, Homo Sapiens
RPPQTLSR

SEQ ID NO: 611 - protein, 9 aa, Homo Sapiens
RQDLEVEVR

SEQ ID NO: 612 - protein, 7 aa, Homo Sapiens
RQEELER

SEQ ID NO: 613 - protein, 8 aa, Homo Sapiens
RQEELERK

SEQ ID NO: 614 - protein, 6 aa, Homo Sapiens
RRQAER

SEQ ID NO: 615 - protein, 16 aa, Homo Sapiens
RTHLGLIMDGWNSMIR

SEQ ID NO: 616 - protein, 8 aa, Homo Sapiens
RTHSPSSK

SEQ ID NO: 617 - protein, 11 aa, Homo Sapiens
RTLEQMDVVHR

SEQ ID NO: 618 - protein, 6 aa, Homo Sapiens
RTPTAR

SEQ ID NO: 619 - protein, 24 aa, Homo Sapiens
SANYTCVAISSLGMIEATAQVTVK

SEQ ID NO: 620 - protein, 14 aa, Homo Sapiens
SCSLVLEHQPDNIK

SEQ ID NO: 621 - protein, 15 aa, Homo Sapiens
SDMGVGVFTPTIEAR

SEQ ID NO: 622 - protein, 8 aa, Homo Sapiens
SDPYGIIR

SEQ ID NO: 623 - protein, 12 aa, Homo Sapiens
SDQALHSFSEAK

SEQ ID NO: 624 - protein, 8 aa, Homo Sapiens
SDSAVLLR

SEQ ID NO: 625 - protein, 12 aa, Homo Sapiens
SDTIANYELVYK

SEQ ID NO: 626 - protein, 13 aa, Homo Sapiens
SEIFNENFGPDFR

SEQ ID NO: 627 - protein, 7 aa, Homo Sapiens
SEIINSK

SEQ ID NO: 628 - protein, 18 aa, Homo Sapiens
SEIQFLISDNQGFSCPEK

SEQ ID NO: 629 - protein, 8 aa, Homo Sapiens
SEQLKPLK

SEQ ID NO: 630 - protein, 9 aa, Homo Sapiens
SEQPTASWR

SEQ ID NO: 631 - protein, 16 aa, Homo Sapiens
SGALQIESSEESDQGK

SEQ ID NO: 632 - protein, 14 aa, Homo Sapiens
SGEGFIDFIGQVHK

SEQ ID NO: 633 - protein, 9 aa, Homo Sapiens
SGVVCQTGR

SEQ ID NO: 634 - protein, 20 aa, Homo Sapiens
SHAENPTASHVDNEYSQPPR

SEQ ID NO: 635 - protein, 12 aa, Homo Sapiens
SHLQNYTVNATK

SEQ ID NO: 636 - protein, 16 aa, Homo Sapiens
SIFDPPVFPVCMLGNR

SEQ ID NO: 637 - protein, 8 aa, Homo Sapiens
SILQEENR

SEQ ID NO: 638 - protein, 9 aa, Homo Sapiens
SIQIHGTMR

SEQ ID NO: 639 - protein, 11 aa, Homo Sapiens
SLEMSHDEHKK

SEQ ID NO: 640 - protein, 11 aa, Homo Sapiens
SLGGEGNFNWR

SEQ ID NO: 641 - protein, 22 aa, Homo Sapiens
SLHLTCDSAPVGSQGTWSTSCR

SEQ ID NO: 642 - protein, 14 aa, Homo Sapiens
SLSGVGAGGGPTPR

SEQ ID NO: 643 - protein, 13 aa, Homo Sapiens
SLSNYPFDFQGAR

SEQ ID NO: 644 - protein, 13 aa, Homo Sapiens
SPDLQGSWQWSDR

SEQ ID NO: 645 - protein, 9 aa, Homo Sapiens
SPGWRPAFK

SEQ ID NO: 646 - protein, 18 aa, Homo Sapiens
SQAKPGTPGGTGGPAPQY

SEQ ID NO: 647 - protein, 17 aa, Homo Sapiens
SQHQETPVYLGATAGMR

SEQ ID NO: 648 - protein, 19 aa, Homo Sapiens
SQHTTEVVGGAPQHEQIGK

SEQ ID NO: 649 - protein, 19 aa, Homo Sapiens
SSHSGVLEIENSVDDLSSR

SEQ ID NO: 650 - protein, 13 aa, Homo Sapiens
SSLPYICEMTAFR

SEQ ID NO: 651 - protein, 14 aa, Homo Sapiens
SSSSNFLNYDLTLR

SEQ ID NO: 652 - protein, 8 aa, Homo Sapiens
SSVINSIR

SEQ ID NO: 653 - protein, 14 aa, Homo Sapiens
SSVISIYVSESMDR

SEQ ID NO: 654 - protein, 15 aa, Homo Sapiens
STEEFPPLQPILQQK

SEQ ID NO: 655 - protein, 8 aa, Homo Sapiens
STETALYR

SEQ ID NO: 656 - protein, 9 aa, Homo Sapiens
STETALYRK

SEQ ID NO: 657 - protein, 14 aa, Homo Sapiens
STGPGASLGTGYDR

SEQ ID NO: 658 - protein, 15 aa, Homo Sapiens
STGPGASLGTGYDRK

SEQ ID NO: 659 - protein, 6 aa, Homo Sapiens
STIEKK

SEQ ID NO: 660 - protein, 18 aa, Homo Sapiens
STTSLSVSWSIPPPQQSR

SEQ ID NO: 661 - protein, 11 aa, Homo Sapiens
SVDDTSQAIQR

SEQ ID NO: 662 - protein, 7 aa, Homo Sapiens
SVGPLTR

SEQ ID NO: 663 - protein, 8 aa, Homo Sapiens
SVGPLTRK

SEQ ID NO: 664 - protein, 12 aa, Homo Sapiens
SVLGVITIVNLR

SEQ ID NO: 665 - protein, 6 aa, Homo Sapiens
SVQMMR

SEQ ID NO: 666 - protein, 5 aa, Homo Sapiens
SYFRK

SEQ ID NO: 667 - protein, 27 aa, Homo Sapiens
SYQLANISSPSLVVECGGQTVQSCVIR

SEQ ID NO: 668 - protein, 9 aa, Homo Sapiens
SYSFVLTNR

SEQ ID NO: 669 - protein, 8 aa, Homo Sapiens
TALLNMDR

SEQ ID NO: 670 - protein, 22 aa, Homo Sapiens
TANPQWNQNITLPAMFPSMCEK

SEQ ID NO: 671 - protein, 21 aa, Homo Sapiens
TAPDLLPHKPLPASAYIEDGR

SEQ ID NO: 672 - protein, 12 aa, Homo Sapiens
TAQSTPSAPPQK

SEQ ID NO: 673 - protein, 13 aa, Homo Sapiens
TASVSINQTEPPK

SEQ ID NO: 674 - protein, 20 aa, Homo Sapiens
TATMLCAAGGNPDPEISWFK

SEQ ID NO: 675 - protein, 20 aa, Homo Sapiens
TATMLCAASGNPDPEITWFK

SEQ ID NO: 676 - protein, 9 aa, Homo Sapiens
TATVVMMTR

SEQ ID NO: 677 - protein, 16 aa, Homo Sapiens
TAVDGPDLEMLTGQER

SEQ ID NO: 678 - protein, 6 aa, Homo Sapiens
TCLEER

SEQ ID NO: 679 - protein, 13 aa, Homo Sapiens
TCSSNLTLTSGSK

SEQ ID NO: 680 - protein, 7 aa, Homo Sapiens
TDAFRRR

SEQ ID NO: 681 - protein, 9 aa, Homo Sapiens
TDALLGEFR

SEQ ID NO: 682 - protein, 11 aa, Homo Sapiens
TDEDVPSGPPR

SEQ ID NO: 683 - protein, 12 aa, Homo Sapiens
TDEDVPSGPPRK

SEQ ID NO: 684 - protein, 12 aa, Homo Sapiens
TDISMSDFENSR

SEQ ID NO: 685 - protein, 9 aa, Homo Sapiens
TDQDEEHCR

SEQ ID NO: 686 - protein, 6 aa, Homo Sapiens
TDVHYR

SEQ ID NO: 687 - protein, 13 aa, Homo Sapiens
TEFQQIINLALQK

SEQ ID NO: 688 - protein, 12 aa, Homo Sapiens
TFGHNTMDAVPR

SEQ ID NO: 689 - protein, 7 aa, Homo Sapiens
TFIDTVR

SEQ ID NO: 690 - protein, 9 aa, Homo Sapiens
TFSDLQSLR

SEQ ID NO: 691 - protein, 10 aa, Homo Sapiens
TFSDLQSLRK

SEQ ID NO: 692 - protein, 13 aa, Homo Sapiens
TGCFIVIDAMLER

SEQ ID NO: 693 - protein, 14 aa, Homo Sapiens
TGEGFIDFIGQVHK

SEQ ID NO: 694 - protein, 11 aa, Homo Sapiens
TGEQAPSSPPR

SEQ ID NO: 695 - protein, 12 aa, Homo Sapiens
TGEQAPSSPPRR

SEQ ID NO: 696 - protein, 15 aa, Homo Sapiens
THLGLIMDGWNSMIR

SEQ ID NO: 697 - protein, 7 aa, Homo Sapiens
THQAFMR

SEQ ID NO: 698 - protein, 8 aa, Homo Sapiens
THQAFMRK

SEQ ID NO: 699 - protein, 12 aa, Homo Sapiens
THSSNSMLVFLK

SEQ ID NO: 700 - protein, 8 aa, Homo Sapiens
TIHAELSK

SEQ ID NO: 701 - protein, 6 aa, Homo Sapiens
TINFAR

SEQ ID NO: 702 - protein, 8 aa, Homo Sapiens
TLADFDPR

SEQ ID NO: 703 - protein, 10 aa, Homo Sapiens
TLEQMDVVHR

SEQ ID NO: 704 - protein, 10 aa, Homo Sapiens
TLHTEELTSK

SEQ ID NO: 705 - protein, 9 aa, Homo Sapiens
TLSAGAGPR

SEQ ID NO: 706 - protein, 27 aa, Homo Sapiens
TMGMVNIFNGDADLSGMTWSHGLSVSK

SEQ ID NO: 707 - protein, 14 aa, Homo Sapiens
TMLHLTDIQLQDNK

SEQ ID NO: 708 - protein, 10 aa, Homo Sapiens
TMPVEQVFAK

SEQ ID NO: 709 - protein, 12 aa, Homo Sapiens
TNEDVPSGPPRK

SEQ ID NO: 710 - protein, 18 aa, Homo Sapiens
TNEPVWEENFTFFIHNPK

SEQ ID NO: 711 - protein, 8 aa, Homo Sapiens
TNHFTIPK

SEQ ID NO: 712 - protein, 14 aa, Homo Sapiens
TNQEVLDFVVGGGR

SEQ ID NO: 713 - protein, 9 aa, Homo Sapiens
TNSILFYGR

SEQ ID NO: 714 - protein, 10 aa, Homo Sapiens
TNVIQSLLAR

SEQ ID NO: 715 - protein, 6 aa, Homo Sapiens
TNWVYR

SEQ ID NO: 716 - protein, 13 aa, Homo Sapiens
TPESSPPGTPIGR

SEQ ID NO: 717 - protein, 12 aa, Homo Sapiens
TPLHAAVAADAR

SEQ ID NO: 718 - protein, 9 aa, Homo Sapiens
TPLSYTHWR

SEQ ID NO: 719 - protein, 6 aa, Homo Sapiens
TPNWDR

SEQ ID NO: 720 - protein, 7 aa, Homo Sapiens
TPNWRPR

SEQ ID NO: 721 - protein, 8 aa, Homo Sapiens
TPPPGGVK

SEQ ID NO: 722 - protein, 19 aa, Homo Sapiens
TPVSTIIMPNEFQQDYDIR

SEQ ID NO: 723 - protein, 19 aa, Homo Sapiens
TQDLENFLCNNLQCGSFLK

SEQ ID NO: 724 - protein, 15 aa, Homo Sapiens
TQEETEDPSVIGEFK

SEQ ID NO: 725 - protein, 21 aa, Homo Sapiens
TQQGVPAQPADFQAEVESDTR

SEQ ID NO: 726 - protein, 18 aa, Homo Sapiens
TQRPAMVQTEDQYQLCYR

SEQ ID NO: 727 - protein, 8 aa, Homo Sapiens
TSGFPAKK

SEQ ID NO: 728 - protein, 16 aa, Homo Sapiens
TSLCVLGPGDEAPLER

SEQ ID NO: 729 - protein, 17 aa, Homo Sapiens
TSLCVLGPGDEAPLERK

SEQ ID NO: 730 - protein, 10 aa, Homo Sapiens
TSSQPGFLER

SEQ ID NO: 731 - protein, 20 aa, Homo Sapiens
TSVLLSWEIPENYNSAMPFK

SEQ ID NO: 732 - protein, 14 aa, Homo Sapiens
TSVLLSWEVPDSYK

SEQ ID NO: 733 - protein, 21 aa, Homo Sapiens
TSVTVSDLEPHMNYTFTVEAR

SEQ ID NO: 734 - protein, 21 aa, Homo Sapiens
TTPPTTRPPPTTTPEPTAPPR

SEQ ID NO: 735 - protein, 12 aa, Homo Sapiens
TVDIYGHVTCMR

SEQ ID NO: 736 - protein, 12 aa, Homo Sapiens
TVDIYGHVTLMR

SEQ ID NO: 737 - protein, 23 aa, Homo Sapiens
TVQTAAANAASTAASSAAQNAFK

SEQ ID NO: 738 - protein, 10 aa, Homo Sapiens
TVSEWLESIK

SEQ ID NO: 739 - protein, 9 aa, Homo Sapiens
TYEVCDVQR

SEQ ID NO: 740 - protein, 9 aa, Homo Sapiens
TYLAVGSMK

SEQ ID NO: 741 - protein, 13 aa, Homo Sapiens
TYQFLQEYLDAIK

SEQ ID NO: 742 - protein, 17 aa, Homo Sapiens
TYVDPHTYEDPNQAVLK

SEQ ID NO: 743 - protein, 8 aa, Homo Sapiens
VAKPEEMK

SEQ ID NO: 744 - protein, 10 aa, Homo Sapiens
VANHMDGFQR

SEQ ID NO: 745 - protein, 13 aa, Homo Sapiens
VAQPGPLEPEEPR

SEQ ID NO: 746 - protein, 12 aa, Homo Sapiens
VATPLPDPMASR

SEQ ID NO: 747 - protein, 20 aa, Homo Sapiens
VDFLGEAGIMGQFSHHNIIR

SEQ ID NO: 748 - protein, 15 aa, Homo Sapiens
VDMTFEEEAQLLQLK

SEQ ID NO: 749 - protein, 11 aa, Homo Sapiens
VDTVAAEHLTR

SEQ ID NO: 750 - protein, 9 aa, Homo Sapiens
VDVGQQPLR

SEQ ID NO: 751 - protein, 11 aa, Homo Sapiens
VEASNPYVEPR

SEQ ID NO: 752 - protein, 8 aa, Homo Sapiens
VEDFDALK

SEQ ID NO: 753 - protein, 11 aa, Homo Sapiens
VEDLPADDILR

SEQ ID NO: 754 - protein, 20 aa, Homo Sapiens
VELPGTAVPSVPEDAAPASR

SEQ ID NO: 755 - protein, 12 aa, Homo Sapiens
VEVEAVNSTSVK

SEQ ID NO: 756 - protein, 16 aa, Homo Sapiens
VEVEPLNSTAVHVYWK

SEQ ID NO: 757 - protein, 16 aa, Homo Sapiens
VEYYIPGSTTNPEVFK

SEQ ID NO: 758 - protein, 15 aa, Homo Sapiens
VFAQNEEIQEMAQNK

SEQ ID NO: 759 - protein, 6 aa, Homo Sapiens
VFHAER

SEQ ID NO: 760 - protein, 7 aa, Homo Sapiens
VFHRPWR

SEQ ID NO: 761 - protein, 9 aa, Homo Sapiens
VFLSIEEFR

SEQ ID NO: 762 - protein, 10 aa, Homo Sapiens
VGEEDDGEYR

SEQ ID NO: 763 - protein, 9 aa, Homo Sapiens
VGFGEEMVK

SEQ ID NO: 764 - protein, 9 aa, Homo Sapiens
VGGSMLTPR

SEQ ID NO: 765 - protein, 9 aa, Homo Sapiens
VGNQIFQSR

SEQ ID NO: 766 - protein, 6 aa, Homo Sapiens
VHGLYR

SEQ ID NO: 767 - protein, 17 aa, Homo Sapiens
VIEEAVYFHQHSILACK

SEQ ID NO: 768 - protein, 6 aa, Homo Sapiens
VIELNK

SEQ ID NO: 769 - protein, 12 aa, Homo Sapiens
VIGAGEFGEVYK

SEQ ID NO: 770 - protein, 19 aa, Homo Sapiens
VLAFTAVGDGPPSPTIQVK

SEQ ID NO: 771 - protein, 12 aa, Homo Sapiens
VLALLCSGFQPK

SEQ ID NO: 772 - protein, 23 aa, Homo Sapiens
VLAPASTLQSSYQIPTENSMTAR

SEQ ID NO: 773 - protein, 16 aa, Homo Sapiens
VLAQQGEYSEAIPILR

SEQ ID NO: 774 - protein, 9 aa, Homo Sapiens
VLAVNSIGR

SEQ ID NO: 775 - protein, 10 aa, Homo Sapiens
VLDAGDPTSR

SEQ ID NO: 776 - protein, 7 aa, Homo Sapiens
VLDSGFR

SEQ ID NO: 777 - protein, 16 aa, Homo Sapiens
VLEDDPEATYTTSGGK

SEQ ID NO: 778 - protein, 17 aa, Homo Sapiens
VLEGMVEENQVNVEVTR

SEQ ID NO: 779 - protein, 7 aa, Homo Sapiens
VLEIPYK

SEQ ID NO: 780 - protein, 7 aa, Homo Sapiens
VLENYNK

SEQ ID NO: 781 - protein, 14 aa, Homo Sapiens
VLHFDQVTENTTEK

SEQ ID NO: 782 - protein, 24 aa, Homo Sapiens
VLMDEGAVLTLVADLSSATLDISK

SEQ ID NO: 783 - protein, 14 aa, Homo Sapiens
VLMEIQDLMFEEMR

SEQ ID NO: 784 - protein, 12 aa, Homo Sapiens
VLVALASEELAK

SEQ ID NO: 785 - protein, 12 aa, Homo Sapiens
VMCVSMGSTTVR

SEQ ID NO: 786 - protein, 20 aa, Homo Sapiens
VNDSNELGGLTTSGSAEVHK

SEQ ID NO: 787 - protein, 10 aa, Homo Sapiens
VPAHQVLFSR

SEQ ID NO: 788 - protein, 8 aa, Homo Sapiens
VPCHFPCK

SEQ ID NO: 789 - protein, 26 aa, Homo Sapiens
VPEDQTGLSGGVASFVCQATGEPKPR

SEQ ID NO: 790 - protein, 8 aa, Homo Sapiens
VPEGFTCR

SEQ ID NO: 791 - protein, 10 aa, Homo Sapiens
VPEGLEDVSK

SEQ ID NO: 792 - protein, 17 aa, Homo Sapiens
VPLSQLLTSEDMTVSQR

SEQ ID NO: 793 - protein, 13 aa, Homo Sapiens
VQCSEQWIPFQNK

SEQ ID NO: 794 - protein, 7 aa, Homo Sapiens
VQVIEGR

SEQ ID NO: 795 - protein, 9 aa, Homo Sapiens
VQWRPQGTR

SEQ ID NO: 796 - protein, 8 aa, Homo Sapiens
VSDFGLSR

SEQ ID NO: 797 - protein, 10 aa, Homo Sapiens
VSTEVTLAVK

SEQ ID NO: 798 - protein, 15 aa, Homo Sapiens
VSWSPAEDHNAPIEK

SEQ ID NO: 799 - protein, 11 aa, Homo Sapiens
VSWVPPPADSR

SEQ ID NO: 800 - protein, 8 aa, Homo Sapiens
VTAAGQTK

SEQ ID NO: 801 - protein, 6 aa, Homo Sapiens
VTEMMK

SEQ ID NO: 802 - protein, 7 aa, Homo Sapiens
VTEMMKK

SEQ ID NO: 803 - protein, 21 aa, Homo Sapiens
VTFDPTSSYTLEDLKPDTLYR

SEQ ID NO: 804 - protein, 20 aa, Homo Sapiens
VTFTCQASFDPSLQPSITWR

SEQ ID NO: 805 - protein, 11 aa, Homo Sapiens
VTLPAGPDLLR

SEQ ID NO: 806 - protein, 9 aa, Homo Sapiens
VTQEIVTER

SEQ ID NO: 807 - protein, 8 aa, Homo Sapiens
VTVAIPLK

SEQ ID NO: 808 - protein, 8 aa, Homo Sapiens
VTYQNHNK

SEQ ID NO: 809 - protein, 8 aa, Homo Sapiens
VVEMQGVR

SEQ ID NO: 810 - protein, 10 aa, Homo Sapiens
VVFQIWDNDK

SEQ ID NO: 811 - protein, 14 aa, Homo Sapiens
VVLLEDLASQVGLR

SEQ ID NO: 812 - protein, 17 aa, Homo Sapiens
VVPSFLPVDQGGSLVGR

SEQ ID NO: 813 - protein, 10 aa, Homo Sapiens
VVQMTNDDIK

SEQ ID NO: 814 - protein, 11 aa, Homo Sapiens
VVQMTNDDIKR

SEQ ID NO: 815 - protein, 7 aa, Homo Sapiens
VVYSAPR

SEQ ID NO: 816 - protein, 24 aa, Homo Sapiens
VYAPASTLVDQPYANEGTVVVTER

SEQ ID NO: 817 - protein, 16 aa, Homo Sapiens
VYDSLLALPQDLQAAR

SEQ ID NO: 818 - protein, 16 aa, Homo Sapiens
VYIDPFTYEDPNEAVR

SEQ ID NO: 819 - protein, 8 aa, Homo Sapiens
VYTENVDK

SEQ ID NO: 820 - protein, 9 aa, Homo Sapiens
VYTENVDKR

SEQ ID NO: 821 - protein, 8 aa, Homo Sapiens
VYTVDLGR

SEQ ID NO: 822 - protein, 5 aa, Homo Sapiens
VYYKK

SEQ ID NO: 823 - protein, 8 aa, Homo Sapiens
VYYTPDSR

SEQ ID NO: 824 - protein, 12 aa, Homo Sapiens
WEDEEWSTDLNR

SEQ ID NO: 825 - protein, 5 aa, Homo Sapiens
WEEHR

SEQ ID NO: 826 - protein, 6 aa, Homo Sapiens
WEEVCR

SEQ ID NO: 827 - protein, 15 aa, Homo Sapiens
WLLLSDPDDFSAGAR

SEQ ID NO: 828 - protein, 12 aa, Homo Sapiens
WLRPSGPMPADR

SEQ ID NO: 829 - protein, 12 aa, Homo Sapiens
WMDWNAPQVQYR

SEQ ID NO: 830 - protein, 10 aa, Homo Sapiens
WMMGAEELTK

SEQ ID NO: 831 - protein, 10 aa, Homo Sapiens
WRPVDLAQVK

SEQ ID NO: 832 - protein, 6 aa, Homo Sapiens
WSNVFK

SEQ ID NO: 833 - protein, 10 aa, Homo Sapiens
WTAPEAIAFR

SEQ ID NO: 834 - protein, 10 aa, Homo Sapiens
WTAPEAISYR

SEQ ID NO: 835 - protein, 11 aa, Homo Sapiens
WTAPEAISYRK

SEQ ID NO: 836 - protein, 5 aa, Homo Sapiens
WTEYR

SEQ ID NO: 837 - protein, 10 aa, Homo Sapiens
WTPPQDSGGR

SEQ ID NO: 838 - protein, 6 aa, Homo Sapiens
WVSQHR

SEQ ID NO: 839 - protein, 12 aa, Homo Sapiens
YAIGVGLAFQNR

SEQ ID NO: 840 - protein, 11 aa, Homo Sapiens
YANVIAYDHSR

SEQ ID NO: 841 - protein, 15 aa, Homo Sapiens
YAVYTVVSSHEQFTK

SEQ ID NO: 842 - protein, 8 aa, Homo Sapiens
YDIEFEDK

SEQ ID NO: 843 - protein, 12 aa, Homo Sapiens
YEGVVDIFQTVK

SEQ ID NO: 844 - protein, 12 aa, Homo Sapiens
YEGVVDMFQTVK

SEQ ID NO: 845 - protein, 6 aa, Homo Sapiens
YEVTYR

SEQ ID NO: 846 - protein, 7 aa, Homo Sapiens
YEVTYRK

SEQ ID NO: 847 - protein, 8 aa, Homo Sapiens
YEVTYRKK

SEQ ID NO: 848 - protein, 9 aa, Homo Sapiens
YFDWILISR

SEQ ID NO: 849 - protein, 10 aa, Homo Sapiens
YFDWILISRR

SEQ ID NO: 850 - protein, 7 aa, Homo Sapiens
YFIEDGR

SEQ ID NO: 851 - protein, 7 aa, Homo Sapiens
YFWTGLR

SEQ ID NO: 852 - protein, 6 aa, Homo Sapiens
YGIQEK

SEQ ID NO: 853 - protein, 18 aa, Homo Sapiens
YGIVLDAGSSHTSLYIYK

SEQ ID NO: 854 - protein, 8 aa, Homo Sapiens
YIAFDFHK

SEQ ID NO: 855 - protein, 9 aa, Homo Sapiens
YIEYQGAEK

SEQ ID NO: 856 - protein, 9 aa, Homo Sapiens
YKPLPQISK

SEQ ID NO: 857 - protein, 18 aa, Homo Sapiens
YKPMMIITEYMENGALDK

SEQ ID NO: 858 - protein, 9 aa, Homo Sapiens
YKPTPIPIK

SEQ ID NO: 859 - protein, 10 aa, Homo Sapiens
YLAEMSYVHR

SEQ ID NO: 860 - protein, 10 aa, Homo Sapiens
YLANMNYVHR

SEQ ID NO: 861 - protein, 32 aa, Homo Sapiens
YLCGAHSDGQLQEGSPIQAWQLFVNEESTIPR

SEQ ID NO: 862 - protein, 13 aa, Homo Sapiens
YLQVSQQLQQTNR

SEQ ID NO: 863 - protein, 16 aa, Homo Sapiens
YMTETWDPSHAPDNFR

SEQ ID NO: 864 - protein, 10 aa, Homo Sapiens
YPDLIAELLR

SEQ ID NO: 865 - protein, 8 aa, Homo Sapiens
YPEVGDLR

SEQ ID NO: 866 - protein, 5 aa, Homo Sapiens
YPWHR

SEQ ID NO: 867 - protein, 6 aa, Homo Sapiens
YQHTGK

SEQ ID NO: 868 - protein, 9 aa, Homo Sapiens
YQVNNLGQR

SEQ ID NO: 869 - protein, 20 aa, Homo Sapiens
YQYFVVDPMAEYNMPQYILR

SEQ ID NO: 870 - protein, 10 aa, Homo Sapiens
YSAPANLYVR

SEQ ID NO: 871 - protein, 10 aa, Homo Sapiens
YSEPPHGLTR

SEQ ID NO: 872 - protein, 15 aa, Homo Sapiens
YSIGGLSPFSEYAFR

SEQ ID NO: 873 - protein, 15 aa, Homo Sapiens
YSVAGLSPYSDYEFR

SEQ ID NO: 874 - protein, 8 aa, Homo Sapiens
YSYNTEWR

SEQ ID NO: 875 - protein, 6 aa, Homo Sapiens
YVCKRK

SEQ ID NO: 876 - protein, 10 aa, Homo Sapiens
YVQNGTYTVK

SEQ ID NO: 877 - protein, 19 aa, Homo Sapiens
AAGSRDVSLAKADAAPDEK

SEQ ID NO: 878 - protein, 20 aa, Homo Sapiens
AEAFGMDPARPDQASTVAVK

SEQ ID NO: 879 - protein, 6 aa, Homo Sapiens
AEDIIK

SEQ ID NO: 880 - protein, 7 aa, Homo Sapiens
AELRGLK

SEQ ID NO: 881 - protein, 20 aa, Homo Sapiens
AGFVGGQFWSVYTPCDTQNK

SEQ ID NO: 882 - protein, 14 aa, Homo Sapiens
ANLSQVADHLDHIK

SEQ ID NO: 883 - protein, 7 aa, Homo Sapiens
AREVIPR

SEQ ID NO: 884 - protein, 8 aa, Homo Sapiens
ATGTAFRR

SEQ ID NO: 885 - protein, 6 aa, Homo Sapiens
AVLGDR

SEQ ID NO: 886 - protein, 9 aa, Homo Sapiens
AVTSPAVGR

SEQ ID NO: 887 - protein, 10 aa, Homo Sapiens
CGVSNKDIEK

SEQ ID NO: 888 - protein, 17 aa, Homo Sapiens
CLNPVPAVPSPSPSVRK

SEQ ID NO: 889 - protein, 11 aa, Homo Sapiens
DCKIRVFIGGK

SEQ ID NO: 890 - protein, 9 aa, Homo Sapiens
DIAPVLDLK

SEQ ID NO: 891 - protein, 11 aa, Homo Sapiens
DIMKKTINFAR

SEQ ID NO: 892 - protein, 9 aa, Homo Sapiens
DPCFHPGYK

SEQ ID NO: 893 - protein, 5 aa, Homo Sapiens
DPELR

SEQ ID NO: 894 - protein, 22 aa, Homo Sapiens
DSPVIDGHNDLPWQLLDMFNNR

SEQ ID NO: 895 - protein, 10 aa, Homo Sapiens
DSQMQNPYSR

SEQ ID NO: 896 - protein, 5 aa, Homo Sapiens
DTYLK

SEQ ID NO: 897 - protein, 6 aa, Homo Sapiens
DVLPQK

SEQ ID NO: 898 - protein, 5 aa, Homo Sapiens
EGIAK

SEQ ID NO: 899 - protein, 8 aa, Homo Sapiens
EIDVSYVK

SEQ ID NO: 900 - protein, 9 aa, Homo Sapiens
ELNEHFKSK

SEQ ID NO: 901 - protein, 6 aa, Homo Sapiens
ELREVR

SEQ ID NO: 902 - protein, 6 aa, Homo Sapiens
ELVSLK

SEQ ID NO: 903 - protein, 7 aa, Homo Sapiens
EQDIADK

SEQ ID NO: 904 - protein, 7 aa, Homo Sapiens
EQLTVIR

SEQ ID NO: 905 - protein, 8 aa, Homo Sapiens
EREAQLVK

SEQ ID NO: 906 - protein, 5 aa, Homo Sapiens
ERMFR

SEQ ID NO: 907 - protein, 6 aa, Homo Sapiens
ESKSIK

SEQ ID NO: 908 - protein, 8 aa, Homo Sapiens
EVCQLLLR

SEQ ID NO: 909 - protein, 5 aa, Homo Sapiens
EVIPR

SEQ ID NO: 910 - protein, 7 aa, Homo Sapiens
FLITRRR

SEQ ID NO: 911 - protein, 13 aa, Homo Sapiens
FLNLTSEKVSQEK

SEQ ID NO: 912 - protein, 9 aa, Homo Sapiens
FSDVTGKIK

SEQ ID NO: 913 - protein, 9 aa, Homo Sapiens
GHEVAAMLK

SEQ ID NO: 914 - protein, 14 aa, Homo Sapiens
GNSAGGLQHRVTAK

SEQ ID NO: 915 - protein, 6 aa, Homo Sapiens
GPGISK

SEQ ID NO: 916 - protein, 18 aa, Homo Sapiens
GTLSPKDALTDLTGDAER

SEQ ID NO: 917 - protein, 16 aa, Homo Sapiens
HIEEENLIDEDFQNLK

SEQ ID NO: 918 - protein, 11 aa, Homo Sapiens
HSRVTVAIPLK

SEQ ID NO: 919 - protein, 5 aa, Homo Sapiens
IDEKR

SEQ ID NO: 920 - protein, 7 aa, Homo Sapiens
IFQALCK

SEQ ID NO: 921 - protein, 4 aa, Homo Sapiens
IIPK

SEQ ID NO: 922 - protein, 5 aa, Homo Sapiens
IKQLR

SEQ ID NO: 923 - protein, 15 aa, Homo Sapiens
ILYDDGKMVEEVDGR

SEQ ID NO: 924 - protein, 15 aa, Homo Sapiens
IRTSTPTGHGASPAK

SEQ ID NO: 925 - protein, 7 aa, Homo Sapiens
ISYRILR

SEQ ID NO: 926 - protein, 5 aa, Homo Sapiens
IYIVR

SEQ ID NO: 927 - protein, 11 aa, Homo Sapiens
KCAQLTVNLTR

SEQ ID NO: 928 - protein, 8 aa, Homo Sapiens
KRSAPTSR

SEQ ID NO: 929 - protein, 4 aa, Homo Sapiens
LALK

SEQ ID NO: 930 - protein, 10 aa, Homo Sapiens
LASSKAHTSR

SEQ ID NO: 931 - protein, 11 aa, Homo Sapiens
LEDPHVDIIRR

SEQ ID NO: 932 - protein, 5 aa, Homo Sapiens
LFGEK

SEQ ID NO: 933 - protein, 9 aa, Homo Sapiens
LKNKEEVLK

SEQ ID NO: 934 - protein, 6 aa, Homo Sapiens
LLDFEK

SEQ ID NO: 935 - protein, 17 aa, Homo Sapiens
LLHGQNGSVPNGQTPLK

SEQ ID NO: 936 - protein, 10 aa, Homo Sapiens
LLIEHGADIR

SEQ ID NO: 937 - protein, 7 aa, Homo Sapiens
LLQAIAK

SEQ ID NO: 938 - protein, 5 aa, Homo Sapiens
LLSDK

SEQ ID NO: 939 - protein, 15 aa, Homo Sapiens
LLSDPNYGVHLPAVK

SEQ ID NO: 940 - protein, 7 aa, Homo Sapiens
LLVPKDR

SEQ ID NO: 941 - protein, 4 aa, Homo Sapiens
LNPK

SEQ ID NO: 942 - protein, 13 aa, Homo Sapiens
LNVEERSVGPLTR

SEQ ID NO: 943 - protein, 24 aa, Homo Sapiens
LQCENVQEIPVFGIVPAIIQTPSR

SEQ ID NO: 944 - protein, 7 aa, Homo Sapiens
LRQGTLR

SEQ ID NO: 945 - protein, 7 aa, Homo Sapiens
LRTSAIR

SEQ ID NO: 946 - protein, 6 aa, Homo Sapiens
LSARNK

SEQ ID NO: 947 - protein, 20 aa, Homo Sapiens
LSDAGQYLCQAGDDSNSNKK

SEQ ID NO: 948 - protein, 9 aa, Homo Sapiens
LSGVEDHVK

SEQ ID NO: 949 - protein, 5 aa, Homo Sapiens
LTVLR

SEQ ID NO: 950 - protein, 9 aa, Homo Sapiens
LTWTNPSIK

SEQ ID NO: 951 - protein, 5 aa, Homo Sapiens
LVLGK

SEQ ID NO: 952 - protein, 14 aa, Homo Sapiens
MRYEGVVDIFQTVK

SEQ ID NO: 953 - protein, 13 aa, Homo Sapiens
MTPPSRAEAGVRR

SEQ ID NO: 954 - protein, 16 aa, Homo Sapiens
MVGDVTGAQAYASTAK

SEQ ID NO: 955 - protein, 19 aa, Homo Sapiens
NADLQVLKPEPELVYEDLR

SEQ ID NO: 956 - protein, 18 aa, Homo Sapiens
NNLVEIILDINVSQLTER

SEQ ID NO: 957 - protein, 5 aa, Homo Sapiens
NPIAK

SEQ ID NO: 958 - protein, 6 aa, Homo Sapiens
NPVDVK

SEQ ID NO: 959 - protein, 6 aa, Homo Sapiens
NQTAVR

SEQ ID NO: 960 - protein, 14 aa, Homo Sapiens
NRAGLGEEFEKEIR

SEQ ID NO: 961 - protein, 8 aa, Homo Sapiens
NRSLSRVR

SEQ ID NO: 962 - protein, 9 aa, Homo Sapiens
QILDQTPVK

SEQ ID NO: 963 - protein, 8 aa, Homo Sapiens
QLVEALDK

SEQ ID NO: 964 - protein, 18 aa, Homo Sapiens
QPHYSAFGSVGEWLRAIK

SEQ ID NO: 965 - protein, 11 aa, Homo Sapiens
RDLGSRLQAQR

SEQ ID NO: 966 - protein, 11 aa, Homo Sapiens
RRDLSQMEALK

SEQ ID NO: 967 - protein, 12 aa, Homo Sapiens
RSSAAGEGTLAR

SEQ ID NO: 968 - protein, 8 aa, Homo Sapiens
SAAASNYV

SEQ ID NO: 969 - protein, 19 aa, Homo Sapiens
SDLQRPNPQSPFCVASSLK

SEQ ID NO: 970 - protein, 9 aa, Homo Sapiens
SLQEANAEK

SEQ ID NO: 971 - protein, 20 aa, Homo Sapiens
SQHQETPVYLGATAGMRLLR

SEQ ID NO: 972 - protein, 9 aa, Homo Sapiens
SQTYESDGK

SEQ ID NO: 973 - protein, 8 aa, Homo Sapiens
SSVKELLK

SEQ ID NO: 974 - protein, 16 aa, Homo Sapiens
STGFTNLGAEGSVFPK

SEQ ID NO: 975 - protein, 16 aa, Homo Sapiens
STGTISVISSGLDREK

SEQ ID NO: 976 - protein, 11 aa, Homo Sapiens
TAIEILAWGLR

SEQ ID NO: 977 - protein, 9 aa, Homo Sapiens
TARATGASR

SEQ ID NO: 978 - protein, 8 aa, Homo Sapiens
TATQPLLK

SEQ ID NO: 979 - protein, 13 aa, Homo Sapiens
TATVVMMTRLEEK

SEQ ID NO: 980 - protein, 11 aa, Homo Sapiens
TELLAADSLSK

SEQ ID NO: 981 - protein, 14 aa, Homo Sapiens
TGRPVEPESEFIIK

SEQ ID NO: 982 - protein, 10 aa, Homo Sapiens
TLAEVCLGQK

SEQ ID NO: 983 - protein, 9 aa, Homo Sapiens
TLRLGPLSK

SEQ ID NO: 984 - protein, 8 aa, Homo Sapiens
TNGTGRVR

SEQ ID NO: 985 - protein, 13 aa, Homo Sapiens
TSKTTFQLELPVK

SEQ ID NO: 986 - protein, 9 aa, Homo Sapiens
TSNGRLPVK

SEQ ID NO: 987 - protein, 9 aa, Homo Sapiens
TVTINCPFK

SEQ ID NO: 988 - protein, 21 aa, Homo Sapiens
VASLIGVEGGHSIDSSLGVLR

SEQ ID NO: 989 - protein, 19 aa, Homo Sapiens
VEASNPYVEPRFLYLGPFK

SEQ ID NO: 990 - protein, 5 aa, Homo Sapiens
VFPGK

SEQ ID NO: 991 - protein, 10 aa, Homo Sapiens
VKCDQYWPAR

SEQ ID NO: 992 - protein, 10 aa, Homo Sapiens
VKTTSLTEKK

SEQ ID NO: 993 - protein, 8 aa, Homo Sapiens
VLAANNVR

SEQ ID NO: 994 - protein, 7 aa, Homo Sapiens
VLDVVER

SEQ ID NO: 995 - protein, 5 aa, Homo Sapiens
VLLEK

SEQ ID NO: 996 - protein, 6 aa, Homo Sapiens
VLTDIK

SEQ ID NO: 997 - protein, 11 aa, Homo Sapiens
VPAHQVLFSRR

SEQ ID NO: 998 - protein, 9 aa, Homo Sapiens
VQAVNSQGK

SEQ ID NO: 999 - protein, 11 aa, Homo Sapiens
VTAAGQTKRTR

SEQ ID NO: 1000 - protein, 6 aa, Homo Sapiens
VTAINK

SEQ ID NO: 1001 - protein, 11 aa, Homo Sapiens
VTLPAGPDILR

SEQ ID NO: 1002 - protein, 6 aa, Homo Sapiens
VVISIR

SEQ ID NO: 1003 - protein, 9 aa, Homo Sapiens
VVNVSDLYK

SEQ ID NO: 1004 - protein, 10 aa, Homo Sapiens
YFTASLPFEK

SEQ ID NO: 1005 - protein, 22 aa, Homo Sapiens
YGPGEPSPVSETVVTPEAAPEK

SEQ ID NO: 1006 - protein, 7 aa, Homo Sapiens
YLNNLYK

SEQ ID NO: 1007 - protein, 14 aa, Homo Sapiens
IGTFCSNGTVSRIK

SEQ ID NO: 1008 - protein, 16 aa, Homo Sapiens
QTPADGEASGESEPAK

SEQ ID NO: 1009 - protein, 18 aa, Homo sapiens
ETGWLLLNKPLDREEIAK

SEQ ID NO: 1010 - protein, 11 aa, Homo sapiens
DPHDLMFTIHR

SEQ ID NO: 1011 - protein, 14 aa, Homo sapiens
STGTISVISSGLDR

SEQ ID NO: 1012 - protein, 16 aa, Homo sapiens
YEAHVPENAVGHEVQR

SEQ ID NO: 1013 - protein, 15 aa, Homo sapiens
LTVTDLDAPNSPAWR

SEQ ID NO: 1014 - protein, 30 aa, Homo sapiens
ATYLIMGGDDGDHFTITTHPESNQGILTTR

SEQ ID NO: 1015 - protein, 18 aa, Homo sapiens
NQHTLYVEVTNEAPFVLK

SEQ ID NO: 1016 - protein, 11 aa, Homo sapiens
QITICNQSPVR

SEQ ID NO: 1017 - protein, 19 aa, Homo sapiens
FLKQDTYDVHLSLSDHGNK

SEQ ID NO: 1018 - protein, 16 aa, Homo sapiens
QDTYDVHLSLSDHGNK

SEQ ID NO: 1019 - protein, 13 aa, Homo sapiens
IKEPLLLPEDDTR

SEQ ID NO: 1020 - protein, 11 aa, Homo sapiens
GLEARPEVVLR

SEQ ID NO: 1021 - protein, 11 aa, Homo sapiens
CAFGYSGLDCK

SEQ ID NO: 1022 - protein, 22 aa, Homo sapiens
CDYYGCNQTADDCLNGLACDCK

SEQ ID NO: 1023 - protein, 10 aa, Homo sapiens
CPDACNAQHK

SEQ ID NO: 1024 - protein, 23 aa, Homo sapiens
DVFGTSVYGQTVILTVSTSLSPR

SEQ ID NO: 1025 - protein, 34 aa, Homo sapiens
HIEEENLIDEDFQNLKLRSTGFTNLGAEGSVFPK

SEQ ID NO: 1026 - protein, 17 aa, Homo sapiens
HSMAYQDLHSEITSLFK

SEQ ID NO: 1027 - protein, 9 aa, Homo sapiens
HSSMPRPDY

SEQ ID NO: 1028 - protein, 15 aa, Homo sapiens
ITASRDSQMQNPYSR

SEQ ID NO: 1029 - protein, 24 aa, Homo sapiens
KSGGAPECACVPGYQEDANGNCQK

SEQ ID NO: 1030 - protein, 18 aa, Homo sapiens
LRSTGFTNLGAEGSVFPK

SEQ ID NO: 1031 - protein, 23 aa, Homo sapiens
SGGAPECACVPGYQEDANGNCQK

SEQ ID NO: 1032 - protein, 18 aa, Homo sapiens
TKHIEEENLIDEDFQNLK

SEQ ID NO: 1033 - DNA, 2899 bp, Homo sapiens
atcatttcctcctcagattaccaagcaagaacagctaaaatgaaagccatcattcatcttactcttcttgctctcctttctgtaaacacagccaccaaccaaggcaactcagctgatgctgtaacaaccacagaaactgcgactagtggtcctacagtagctgcagctgataccactgaaactaatttccctgaaactgctagcaccacagcaaatacaccttctttcccaacagctacttcacctgctccccccataattagtacacatagttcctccacaattcctacacctgctccccccataattagtacacatagttcctccacaattcctatacctactgctgcagacagtgagtcaaccacaaatgtaaattcattagctacctctgacataatcaccgcttcatctccaaatgatggattaatcacaatggttccttctgaaacacaaagtaacaatgaaatgtcccccaccacagaagacaatcaatcatcagggcctcccactggcaccgctttattggagaccagcaccctaaacagcacaggtcccagcaatccttgccaagatgatccctgtgcagataattcgttatgtgttaagctgcataatacaagtttttgcctgtgtttagaagggtattactacaactcttctacatgtaagaaaggaaaggtattccctgggaagatttcagtgacagtatcagaaacatttgacccagaagagaaacattccatggcctatcaagacttgcatagtgaaattactagcttgtttaaagatgtatttggcacatctgtttatggacagactgtaattcttactgtaagcacatctctgtcaccaagatctgaaatgcgtgctgatgacaagtttgttaatgtaacaatagtaacaattttggcagaaaccacaagtgacaatgagaagactgtgactgagaaaattaataaagcaattagaagtagctcaagcaactttctaaactatgatttgacccttcggtgtgattattatggctgtaaccagactgcggatgactgcctcaatggtttagcatgcgattgcaaatctgacctgcaaaggcctaacccacagagccctttctgcgttgcttccagtctcaagtgtcctgatgcctgcaacgcacagcacaagcaatgcttaataaagaagagtggtggggcccctgagtgtgcgtgcgtgcccggctaccaggaagatgctaatgggaactgccaaaagtgtgcatttggctacagtggactcgactgtaaggacaaatttcagctgatcctcactattgtgggcaccatcgctggcattgtcattctcagcatgataattgcattgattgtcacagcaagatcaaataacaaaacgaagcatattgaagaagagaacttgattgacgaagactttcaaaatctaaaactgcggtcgacaggcttcaccaatcttggagcagaagggagcgtctttcctaaggtcaggataacggcctccagagacagccagatgcaaaatccctattcaagacacagcagcatgccccgccctgactattagaatcataagaatgtggaacccgccatggcccccaaccaatgtacaagctattatttagagtgtttagaaagactgatggagaagtgagcaccagtaaagatctggcctccggggtttttcttccatctgacatctgccagcctctctgaatggaagttgtgaatgtttgcaacgaatccagctcacttgctaaataagaatctatgacattaaatgtagtagatgctattagcgcttgtcagagaggtggttttcttcaatcagtacaaagtactgagacaatggttagggttgttttcttaattcttttcctggtagggcaacaagaaccatttccaatctagaggaaagctccccagcattgcttgctcctgggcaaacattgctcttgagttaagtgacctaattcccctgggagacatacgcatcaactgtggaggtccgaggggatgagaagggatacccaccatctttcaagggtcacaagctcactctctgacaagtcagaatagggacactgcttctatccctccaatggagagattctggcaacctttgaacagcccagagcttgcaacctagcctcacccaagaagactggaaagagacatatctctcagctttttcaggaggcgtgcctgggaatccaggaactttttgatgctaattagaaggcctggactaaaaatgtccactatggggtgcactctacagtttttgaaatgctaggaggcagaaggggcagagagtaaaaaacatgacctggtagaaggaagagaggcaaaggaaactgggtggggaggatcaattagagaggaggcacctgggatccaccttcttccttaggtcccctcctccatcagcaaaggagcacttctctaatcatgccctcccgaagactggctgggagaaggtttaaaaacaaaaaatccaggagtaagagccttaggtcagtttgaaattggagacaaactgtctggcaaagggtgcgagagggagcttgtgctcaggagtccagccgtccagcctcggggtgtaggtttctgaggtgtgccattggggcctcagccttctctggtgacagaggctcagctgtggccaccaacacacaaccacacacacacaaccacacacacaaatgggggcaaccacatccagtacaagcttttacaaatgttattagtgtccttttttatttctaatgccttgtcctcttaaaagttattttatttgttattattatttgttcttgactgttaattgtgaatggtaatgcaataaagtgcctttgttagatggtgaaaaaaaaaaaaaaaaaaaa

SEQ ID NO: 1034 - DNA, 4275 bp, Homo sapiens
gcgttttaaaaattgtctttatttacattttacagaaagttgagaaagtgttatttatatggggggtaggggtgctggagattatgagactaataacaaccctcttagctcgcaccctttggcaccactacagcttccaaactctgggactttctcgactagcttccctttgtttagctgtgaaatggaagaagcggtccgggtgtggcggctcatgcctgtaacctgagcactctgggaggcggaggatcgcttgagtccagaagttcaagaccagcttgggcaacatagggtgaccctccaccctccccccgccccaccacatcgctacaaaaaatttttaaaaattagccgggtgtggtggcgcaagcctgtagtctcagcgggagctgagggaggagaatcgcttcagcccgggaggtcgaggctgtagtgagccgagatcgcgctactgcactcctgggcgacagagcgagaccctgtctccaaaaaaaaaaaaaaaagaaaaaagaggaagttgtatccaattcagaaacgcggtccttcgggacctgctagttttataccccggaggatcctccccggcgggctggcacgggaggtggagaaagaggcttgggcggccccgctgtagccgcgtgtgggaggacgcacgggcctgcttcaaagctttgggataacagcgcctccgggggataatgaatgcggagcctccgttttcagtcgacttcagatgtgtctccacttttttccgctgtagccgcaaggcaaggaaacatttctcttcccgtactgaggaggctgaggagtgcactgggtgttcttttctcctctaacccagaactgcgagacagaggctgagtccctgtaaagaacagctccagaaaagccaggagagcgcaggagggcatccgggaggccaggaggggttcgctggggcctcaaccgcacccacatcggtcccacctgcgagggggcgggacctcgtggcgctggaccaatcagcacccacctgcgctcacctggcctcctcccgctggctcccgggggctgcggtgctcaaaggggcaagagctgagcggaacaccggcccgccgtcgcggcagctgcttcacccctctctctgcagccatggggctccctcgtggacctctcgcgtctctcctccttctccaggtttgctggctgcagtgcgcggcctccgagccgtgccgggcggtcttcagggaggctgaagtgaccttggaggcgggaggcgcggagcaggagcccggccaggcgctggggaaagtattcatgggctgccctgggcaagagccagctctgtttagcactgataatgatgacttcactgtgcggaatggcgagacagtccaggaaagaaggtcactgaaggaaaggaatccattgaagatcttcccatccaaacgtatcttacgaagacacaagagagattgggtggttgctccaatatctgtccctgaaaatggcaagggtcccttcccccagagactgaatcagctcaagtctaataaagatagagacaccaagattttctacagcatcacggggccgggggcagacagcccccctgagggtgtcttcgctgtagagaaggagacaggctggttgttgttgaataagccactggaccgggaggagattgccaagtatgagctctttggccacgctgtgtcagagaatggtgcctcagtggaggaccccatgaacatctccatcatcgtgaccgaccagaatgaccacaagcccaagtttacccaggacaccttccgagggagtgtcttagagggagtcctaccaggtacttctgtgatgcaggtgacagccacggatgaggatgatgccatctacacctacaatggggtggttgcttactccatccatagccaagaaccaaaggacccacacgacctcatgttcaccattcaccggagcacaggcaccatcagcgtcatctccagtggcctggaccgggaaaaagtccctgagtacacactgaccatccaggccacagacatggatggggacggctccaccaccacggcagtggcagtagtggagatccttgatgccaatgacaatgctcccatgtttgacccccagaagtacgaggcccatgtgcctgagaatgcagtgggccatgaggtgcagaggctgacggtcactgatctggacgcccccaactcaccagcgtggcgtgccacctaccttatcatgggcggtgacgacggggaccattttaccatcaccacccaccctgagagcaaccagggcatcctgacaaccaggaagggtttggattttgaggccaaaaaccagcacaccctgtacgttgaagtgaccaacgaggccccttttgtgctgaagctcccaacctccacagccaccatagtggtccacgtggaggatgtgaatgaggcacctgtgtttgtcccaccctccaaagtcgttgaggtccaggagggcatccccactggggagcctgtgtgtgtctacactgcagaagaccctgacaaggagaatcaaaagatcagctaccgcatcctgagagacccagcagggtggctagccatggacccagacagtgggcaggtcacagctgtgggcaccctcgaccgtgaggatgagcagtttgtgaggaacaacatctatgaagtcatggtcttggccatggacaatggaagccctcccaccactggcacgggaacccttctgctaacactgattgatgtcaatgaccatggcccagtccctgagccccgtcagatcaccatctgcaaccaaagccctgtgcgccaggtgctgaacatcacggacaaggacctgtctccccacacctcccctttccaggcccagctcacagatgactcagacatctactggacggcagaggtcaacgaggaaggtgacacagtggtcttgtccctgaagaagttcctgaagcaggatacatatgacgtgcacctttctctgtctgaccatggcaacaaagagcagctgacggtgatcagggccactgtgtgcgactgccatggccatgtcgaaacctgccctggaccctggaagggaggtttcatcctccctgtgctgggggctgtcctggctctgctgttcctcctgctggtgctgcttttgttggtgagaaagaagcggaagatcaaggagcccctcctactcccagaagatgacacccgtgacaacgtcttctactatggcgaagaggggggtggcgaagaggaccaggactatgacatcacccagctccaccgaggtctggaggccaggccggaggtggttctccgcaatgacgtggcaccaaccatcatcccgacacccatgtaccgtcctcggccagccaacccagatgaaatcggcaactttataattgagaacctgaaggcggctaacacagaccccacagccccgccctacgacaccctcttggtgttcgactatgagggcagcggctccgacgccgcgtccctgagctccctcacctcctccgcctccgaccaagaccaagattacgattatctgaacgagtggggcagccgcttcaagaagctggcagacatgtacggtggcggggaggacgactaggcggcctgcctgcagggctggggaccaaacgtcaggccacagagcatctccaaggggtctcagttcccccttcagctgaggacttcggagcttgtcaggaagtggccgtagcaacttggcggagacaggctatgagtctgacgttagagtggtggcttccttagcctttcaggatggaggaatgtgggcagtttgacttcagcactgaaaacctctccacctgggccagggttgcctcagaggccaagtttccagaagcctcttacctgccgtaaaatgctcaaccctgtgtcctgggcctgggcctgctgtgactgacctacagtggactttctctctggaatggaaccttcttaggcctcctggtgcaacttaatttttttttttaatgctatcttcaaaacgttagagaaagttcttcaaaagtgcagcccagagctgctgggcccactggccgtcctgcatttctggtttccagaccccaatgcctcccattcggatggatctctgcgtttttatactgagtgtgcctaggttgccccttattttttattttccctgttgcgttgctatagatgaagggtgaggacaatcgtgtatatgtactagaacttttttattaaagaaacttttcccagaggtgcctggggagtg